(19) United States
(12) Patent Application Publication
(10) Pub. No.: US 2026/0183426 A1
(43) Pub. Date: Jul. 2, 2026

(54) COMPOSITIONS AND METHODS FOR MODULATING CXCL9, CXCL10, AND CXCL11 GENE EXPRESSION

(71) Applicant: Flagship Labs 114, Inc., Cambridge, MA (US)

(72) Inventors: Justin Chen, Cambridge, MA (US); Jeremiah D. Farelli, Marblehead, MA (US); Charles O'Donnell, Winchester, MA (US); Amy Theresa McCurley, Cambridge, MA (US)

(73) Assignee: Flagship Labs 114, Inc., Cambridge, MA (US)

(21) Appl. No.: 19/396,598

(22) Filed: Nov. 21, 2025

Related U.S. Application Data
(63) Continuation of application No. PCT/US2024/030840, filed on May 23, 2024.
(60) Provisional application No. 63/468,458, filed on May 23, 2023.

(51) Int. Cl. A61K 48/00; A61K 9/127; A61K 9/51; A61P 37/06; C12N 9/22; C12N 15/11; C12N 15/86; C12N 15/88

(52) U.S. Cl. CPC A61K 48/005; A61K 9/127; A61K 9/5123; A61P 37/06; C12N 9/222; C12N 15/111; C12N 15/86; C12N 15/88; C07K 2319/81; C12N 2310/20; C12Y 301/11001

(57) ABSTRACT

The present invention provides agents and compositions for reducing expression of a CXCL9, CXCL10, and/or CXCL11 gene by targeting expression control region associated with CXCL9, CXCL10, and/or CXCL11 gene and methods of use thereof for treating a disease or disorder associated with CXCL9, CXCL10, and/or CXCL11, e.g., liver disease.

### CROSS-REFERENCE TO RELATED APPLICATIONS

This application is a continuation of PCT International Application Serial No. PCT/US2024/030840, filed on May 23, 2024, which application claims the benefit of priority to U.S. Provisional Application No. 63/468,458, filed on May 23, 2023. The entire contents of the prior applications are hereby incorporated herein by reference in their entireity.

### REFERENCE TO ELECTRONIC SEQUENCE LISTING

The application contains a Sequence Listing which has been submitted electronically in .XML format and is hereby incorporated by reference in its entirety. Said .XML copy, created on May 20, 2024, is named “131717-01420.xml” and is 444,973 bytes in size. The sequence listing contained in this .XML file is part of the specification and is hereby incorporated by reference herein in its entirety.

### BACKGROUND OF THE INVENTION

Hepatitis is a broad pathophysiological feature common to acute and chronic liver diseases characterized by activation of inflammatory mechanisms, including increased interferon gamma (IFNG) expression. IFNG is a pleotropic proinflammatory cytokine released by immune cells that contributes to liver injury by stimulating the release of chemokines CXCL9, CXCL10 and CXCL11 from hepatocytes. CXCL9-10-11 bind CXCR3 on T cells and promote their recruitment to the site of inflammation.

### SUMMARY OF THE INVENTION

The present invention provides agents and compositions for reducing expression of a CXCL9, CXCL10, and/or CXCL11 gene by targeting a CXCL9, CXCL10, or CXCL11 expression control region. The CXCL9, CXCL10, and/or CXCL11 gene may be in a cell, e.g., a mammalian cell, such as a mammalian somatic cell, e.g., a human somatic cell. The present invention also provides methods of using the agents and compositions of the invention for reducing the expression of a CXCL9, CXCL10, and/or CXCL11 gene or for treating a subject who would benefit from reducing the expression of a CXCL9, CXCL10, and/or CXCL11 gene, e.g., a subject suffering or prone to suffering from a CXCL9, CXCL10, and/or CXCL11-associated disease e.g., liver disease, e.g., inflammatory liver disease.

In one aspect, the disclosure provides a site-specific epigenetic modifying agent, comprising at least one site-specific targeting moiety which targets a target sequence within an expression control region of human CXCL9, CXCL10, or CXCL11.

In some embodiments, the target sequence is within a human chromosomal region selected from the group consisting of chr4:76928543-76931536, chr4:76944524-76948668, chr4:76957098-76962383, chr4:76949523-76949873, chr4:76932906-76933929, chr4:76916900-76917300, chr4:76988761-76989260, chr4:77008274-77008727, and chr4:76957852-76958551.

In some embodiments, the target sequence is located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb from a human chromosomal region selected from the group consisting of chr4:76958201-76958221, chr4:76949758-76949778, chr4:76933245-76933265, chr4:76932967-76932987, chr4:76957115-76957132, chr4:76944734-76944751, chr4:76944656-76944673, and chr4:76933040-76933057.

In some embodiments, the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

In some embodiments, at least two site specific targeting moieties, each of which independently targets CXCL9, CXCL10 or CXCL11.

In some embodiments, the site-specific targeting moiety comprises a polymeric molecule.

In some embodiments, the polymeric molecule comprises a polyamide.

In some embodiments, the polymeric molecule comprises a polynucleotide.

In some embodiments, the expression control region comprises at least one specific transcriptional control element for CXCL9, CXCL10, or CXCL11.

In some embodiments, comprising at least two specific transcriptional control elements, each of which independently reduces transcription of CXCL9, CXCL10 or CXCL11.

In some embodiments, the transcriptional control element comprises a promoter for CXCL9, CXCL10, or CXCL11.

In some embodiments, the transcriptional control element comprises an enhancer for CXCL9, CXCL10, or CXCL11.

In some embodiments, the site-specific targeting moiety comprises a nucleotide sequence having at least 85%, 90%, 95%, 96%, 97%, 98% or 99% nucleotide identity to the entire nucleotide sequence of any of the nucleotide sequences in any one of Table 3 or Table 5.

In some embodiments, the polymeric molecule comprises a polynucleotide encoding a Cas or dCas polypeptide, or a fragment thereof, and a polynucleotide encoding a guide RNA that specifically binds to the expression control region of CXCL9, CXCL10, or CXCL11; or a DNA-binding domain of a Transcription activator-like effector (TALE) polypeptide or a zinc finger (ZFP) polypeptide, or fragment thereof, that specifically binds to the expression control region of CXCL9, CXCL10, or CXCL11.

In some embodiments, the DNA-binding domain of the ZFP or TALE polypeptide comprises an amino acid sequence having at least about 85%, 90%, 95%, 96%, 97%, 98% or 99% amino acid identity to the entire amino acid sequence of any one of the amino acid sequences listed in Table 3 or Table 5.

In some embodiments, the expression control region comprises 1) one or more CXCL9-associated anchor sequences within an anchor sequence-mediated conjunction comprising a first and a second CXCL9-associated anchor sequence; 2) one or more CXCL10-associated anchor sequences within an anchor sequence-mediated conjunction comprising a first and a second CXCL10-associated anchor sequence; or 3) one or more CXCL11-associated anchor sequences within an anchor sequence-mediated conjunction comprising a first and a second CXCL11-associated anchor sequence.

In some embodiments, the expression control region comprises one or more CCCTC-binding factor (CTCF) binding motifs.

In some embodiments, the anchor sequence-mediated conjunction comprises one or more transcriptional control elements internal to the conjunction.

In some embodiments, the anchor sequence-mediated conjunction comprises one or more transcriptional control elements external to the conjunction.

In some embodiments, the first and/or the second anchor sequence is located within about 500 kb of the transcriptional control element.

In some embodiments, the first and/or the second anchor sequence is located within about 300 kb of the transcriptional control element.

In some embodiments, the first and/or the second anchor sequence is located within 10 kb of the transcriptional control element.

In some embodiments, the polymeric molecule comprises a polynucleotide encoding a DNA-binding domain, or fragment thereof, of a zinc finger polypeptide (ZFP) or a transcription activator-like effector (TALE) polypeptide that specifically binds to the expression control region of CXCL9, CXCL10, or CXCL11.

In some embodiments, the DNA-binding domain of the ZFP or TALE polypeptide comprises an amino acid sequence having at least about 85%, 90%, 95%, 96%, 97%, 98% or 99% amino acid identity to the entire amino acid sequence of any one of the amino acid sequences listed in Table 3 or Table 5.

In some embodiments, the epigenetic modifying agent comprises a nucleotide modification.

In some embodiments, the polymeric molecule comprises a peptide nucleic acid (PNA).

In one aspect, the disclosure provides a nucleic acid molecule, wherein the nucleic acid molecule encodes the epigenetic modifying agent of the above aspects or any other aspect of the invention delineated herein.

In one aspect, the disclosure provides a vector comprising the epigenetic modifying agent of the above aspects or any other aspect of the invention delineated herein.

In some embodiments, the vector is a viral expression vector.

In one aspect, the disclosure provides a cell comprising the epigenetic modifying agent of the above aspects or any other aspect of the invention delineated herein or the vector of the above aspects or any other aspect of the invention delineated herein.

In one aspect, the disclosure provides a composition comprising the epigenetic modifying agent of the above aspects or any other aspect of the invention delineated herein.

In some embodiments, the composition comprises at least two epigenetic modifying agents, each of which independently targets an expression control region of one of CXCL9, CXCL10, or CXCL11.

In some embodiments, the composition comprises at least three epigenetic modifying agents, each of which targets an expression control region of one of CXCL9, CXCL10, or CXCL11.

In some embodiments, the composition comprises an epigenetic modifying agent which targets the expression control region of at least two of CXCL9, CXCL10, or CXCL11.

In some embodiments, the composition comprises a pharmaceutical composition.

In some embodiments, the pharmaceutical composition comprises a lipid formulation.

In some embodiments, the lipid formulation comprises one or more cationic lipids, one or more non-cationic lipids, one or more cholesterol-based lipids, or one or more PEG-modified lipids, or combinations of any of the foregoing.

In some embodiments, the pharmaceutical composition comprises a lipid nanoparticle.

In one aspect, the disclosure provides an epigenetic modifying agent, comprising at least one nucleic acid molecule encoding a fusion protein, the fusion protein comprising a site-specific targeting moiety which targets a target sequence within an expression control region of human CXCL9, CXCL10, or CXCL11, and an effector molecule.

In some embodiments, the target sequence is within a human chromosomal region selected from the group consisting of chr4: 76928543-76931536, chr4: 76944524-76948668, chr4: 76957098-76962383, chr4: 76949523-76949873, chr4: 76932906-76933929, chr4: 76916900-76917300, chr4: 76988761-76989260, chr4: 77008274-77008727, and chr4: 76957852-76958551.

In some embodiments, target sequence is located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb from a human chromosomal region selected from the group consisting of chr4: 76958201-76958221, chr4: 76949758-76949778, chr4: 76933245-76933265, chr4: 76932967-76932987, chr4: 76957115-76957132, chr4: 76944734-76944751, chr4: 76944656-76944673, and chr4: 76933040-76933057.

In some embodiments, the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

In some embodiments, the site-specific targeting moiety comprises a polynucleotide encoding a Cas or dCas polypeptide, or a fragment thereof, and a polynucleotide encoding a guide RNA that specifically binds to the expression control region of CXCL9, CXCL10, or CXCL11; or a DNA-binding domain of a Transcription activator-like effector (TALE) polypeptide or a zinc finger (ZFP) polypeptide, or fragment thereof, that specifically binds to the expression control region of CXCL9, CXCL10, or CXCL11.

In some embodiments, the DNA-binding domain of the zinc finger polypeptide (ZFP) or TALE polypeptide comprises an amino acid sequence having at least 85%, 90%, 95%, 96%, 97%, 98% or 99% amino acid identity to the entire amino acid sequence of an amino acid sequence selected from the amino acid sequences listed in Table 3 or Table 5.

In some embodiments, the effector molecule comprises a polypeptide or a nucleic acid molecule encoding a polypeptide.

In some embodiments, the fusion protein comprises a peptide-nucleic acid fusion.

In some embodiments, the effector is selected from the group consisting of a nuclease, a physical blocker, an epigenetic recruiter, an epigenetic modifier, and combinations of any of the foregoing.

In some embodiments, the effector is selected from the group consisting of euchromatic histone-lysine N-methyltransferase 2 (G9a), enhancer of zeste homolog 2 (EZH2), MQ1 domain (MQ1), Krüppel associated box (KRAB) transcriptional repression domain, and histone deacetylase 8 (HDAC8).

In some embodiments, the effector is MQ1.

In some embodiments, the effector comprises a CRISPR associated protein (Cas) polypeptide or nucleic acid molecule encoding the Cas polypeptide.

In some embodiments, the Cas polypeptide is an enzymatically inactive Cas polypeptide.

In some embodiments, further comprising a catalytically active domain of human exonuclease 1 (hEXO1).

In some embodiments, the epigenetic modifier comprises a transcriptional repressor.

In some embodiments, the transcriptional repressor is selected from the group comprising euchromatic histone-lysine N-methyltransferase 2 (G9a), enhancer of zeste homolog 2 (EZH2), MQ1 domain (MQ1), Krüppel associated box (KRAB) transcriptional repression domain, and histone deacetylase 8 (HDAC8).

In some embodiments, the transcriptional repressor is MQ1 domain.

In some embodiments, the MQ1 domain comprises an amino acid sequence having at least about 85%, 90%, 95%, 96%, 97%, 98% or 99% amino acid identity to the entire amino acid sequence of SEQ ID NO: 79.

In some embodiments, the transcriptional repressor comprises two, three, four, or five MQ1s.

In some embodiments, the transcriptional repressor is a EZH2.

In some embodiments, the EZH2 comprises an amino acid sequence having at least about 85%, 90%, 95%, 96%, 97%, 98% or 99% identity to the entire amino acid sequence of SEQ ID NO: 78.

In some embodiments, the epigenetic modifier comprises a DNA methylase or a histone deacetylase.

In some embodiments, the effector molecule comprises a Krüppel associated box (KRAB) transcriptional repression domain.

In some embodiments, the KRAB comprises an amino acid sequence having at least about 85%, 90%, 95%, 96%, 97%, 98% or 99% identity to the entire amino acid sequence of SEQ ID NO: 297.

In some embodiments, the effector molecule comprises a Transcription activator-like effector nuclease (TALEN) polypeptide.

In some embodiments, further comprising a second nucleic acid molecule encoding a second fusion protein, wherein 1) the second fusion protein comprises a second site-specific targeting moiety which targets a second expression control region of CXCL9, CXCL10 or CXCL11, which is the same or different than the target of the first site-specific targeting moiety; 2) the second fusion protein comprises a second site-specific targeting moiety which targets a second CXCL9 expression control region and a second effector molecule, wherein the second CXCL9 expression control region is different than the CXCL9 expression control region; 3) the second fusion protein comprises a second site-specific targeting moiety which targets a second CXCL10 expression control region and a second effector molecule, wherein the second CXCL10 expression control region is different than the CXCL10 expression control region; and/or 4) the second fusion protein comprises a second site-specific targeting moiety which targets a second CXCL11 expression control region and a second effector molecule, wherein the second CXCL11 expression control region is different than the CXCL11 expression control region.

In some embodiments, the first nucleic acid molecule and the second nucleic acid molecule are located on the same nucleic acid molecule or on different nucleic acid molecules.

In some embodiments, 1) the epigenetic modifying agent targets a CXCL9 expression control region comprising either a ZF target sequence comprising SEQ ID NOs: 422-425, or TALE target sequence comprising SEQ ID NOs: 429-432, or a combination thereof, and a second CXCL9 expression control region comprising either a ZF target sequence comprising SEQ ID NOs: 422-425, or TALE target sequence comprising SEQ ID NOs: 429-432, or a combination thereof; and/or 2) the epigenetic modifying agent targets a CXCL10 expression control region comprising either a ZF target sequence comprising SEQ ID NOs: 422-425, or TALE target sequence comprising SEQ ID NOs: 429-432, or a combination thereof, and a second CXCL10 expression control region comprising either a ZF target sequence comprising SEQ ID NOs: 422-425, or TALE target sequence comprising SEQ ID NOs: 429-432, or a combination thereof; and/or 3) the epigenetic modifying agent targets a CXCL11 expression control region comprising either a ZF target sequence comprising SEQ ID NOs: 422-425, or TALE target sequence comprising SEQ ID NOs: 429-432, or a combination thereof, a second CXCL11 expression control region comprising either a ZF target sequence comprising SEQ ID NOs: 422-425, or TALE target sequence comprising SEQ ID NOs: 429-432, or a combination thereof.

In some embodiments, the second effector is different than the effector.

In some embodiments, the second effector is the same as the effector.

In some embodiments, the fusion protein and the second fusion protein are operably linked.

In some embodiments, the fusion protein, and, optionally, the second fusion protein independently comprise an amino acid sequence that has at least about 85%, 90%, 95%, 96%, 97%, 98% or 99% amino acid sequence identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

In some embodiments, the fusion protein, and optionally, the second fusion protein, are independently encoded by a polynucleotide comprising a nucleotide sequence that has at least about 85%, 90%, 95%, 96%, 97%, 98% or 99% nucleotide sequence identity to the entire nucleotide sequence of a polynucleotide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

In one aspect, the disclosure provides an epigenetic modifying agent, comprising a nucleic acid molecule encoding a fusion protein, wherein the fusion protein comprises an amino acid sequence having at least about 85%, 90%, 95%, 96%, 97%, 98% or 99% amino acid identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

In some embodiments, the polypeptide is ZF05-KRAB.

In some embodiments, the polypeptide is ZF07-KRAB.

In some embodiments, the polypeptide is ZF16-KRAB.

In some embodiments, the polypeptide is ZF24-KRAB.

In some embodiments, the polypeptide is ZF05-MQ1.

In some embodiments, the polypeptide is ZF07-MQ1.

In some embodiments, the polypeptide is TAL04-KRAB.

In some embodiments, the polypeptide is TAL08-KRAB.

In some embodiments, the polypeptide is TAL09-KRAB.

In some embodiments, the polypeptide is TAL14-KRAB.

In one aspect, the disclosure provides an epigenetic modifying agent, comprising a nucleic acid molecule encoding two or more fusion proteins, wherein each fusion protein independently comprises an amino acid sequence having at least about 85%, 90%, 95%, 96%, 97%, 98% or 99% amino acid identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

In one aspect, the disclosure provides a vector comprising a nucleic acid molecule encoding the epigenetic modifying agent of the above aspects or any other aspect of the invention delineated herein.

In some embodiments, the vector is a viral expression vector.

In one aspect, the disclosure provides a cell comprising the epigenetic modifying agent of the above aspects or any other aspect of the invention delineated herein or the vector of the above aspects or any other aspect of the invention delineated herein.

In some embodiments, the cell is an immune cell.

In one aspect, the disclosure provides a composition comprising the epigenetic modifying agent of the above aspects or any other aspect of the invention delineated herein.

In some embodiments, the composition comprises at least one epigenetic modifying agent that targets an expression control region of CXCL9, CXCL10, or CXCL11.

In some embodiments, the composition comprises at least two epigenetic modifying agent, each of which independently targets an expression control region of CXCL9, CXCL10, or CXCL11.

In some embodiments, the composition comprises at least three epigenetic modifying agent, each of which targets an expression control region of CXCL9, CXCL10, or CXCL11.

In some embodiments, the composition comprises a pharmaceutical composition.

In some embodiments, the pharmaceutical composition comprises a lipid formulation.

In some embodiments, the lipid formulation comprises one or more cationic lipids, one or more non-cationic lipids, one or more cholesterol-based lipids, or one or more PEG-modified lipids, or combinations of any of the foregoing.

In some embodiments, the pharmaceutical composition comprises a lipid nanoparticle.

In one aspect, the disclosure provides a method of reducing expression of CXCL9, CXCL10, or CXCL11 in a cell, the method comprising contacting the cell with at least one epigenetic modifying agent, each epigenetic modifying agent comprising at least one site-specific targeting moiety which targets a target sequence within an expression control region of human CXCL9, CXCL10, or CXCL11, and an effector molecule, thereby reducing expression of human CXCL9, CXCL10, or CXCL11 in the cell.

In some embodiments, the target sequence is within a human chromosomal region selected from the group consisting of chr4: 76928543-76931536, chr4: 76944524-76948668, chr4: 76957098-76962383, chr4: 76949523-76949873, chr4: 76932906-76933929, chr4: 76916900-76917300, chr4: 76988761-76989260, chr4: 77008274-77008727, and chr4: 76957852-76958551.

In some embodiments, the target sequence is located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb from a human chromosomal region selected from the group consisting of chr4: 76958201-76958221, chr4: 76949758-76949778, chr4: 76933245-76933265, chr4: 76932967-76932987, chr4: 76957115-76957132, chr4: 76944734-76944751, chr4: 76944656-76944673, and chr4: 76933040-76933057.

In some embodiments, the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

In some embodiments, the epigenetic modifying agent comprises at least two site specific targeting moieties, each of which independently targets CXCL9, CXCL10 or CXCL11.

In some embodiments, the expression of CXCL9, CXCL10, or CXCL11 in the cell is reduced by at least 30%, at least 35%, at least 40%, at least 45%, at least 50%, at least 55%, at least 60%, at least 65%, at least 70%, at least 75%, at least 80%, at least 85%, at least 90%, or at least 95% compared to prior to contacting the cell with the at least one epigenetic modifying agent.

In some embodiments, the expression of CXCL9, CXCL10, or CXCL11 in the cell is reduced between about 30% to about 100%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 35% to about 100%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 40% to about 100%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 100%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 100%, about 55% to about 95%, about 55% to about 90%, about 55% to about 85%, about 55% to about 80%, about 55% to about 75%, about 55% to about 70%, about 55% to about 65%, about 55% to about 60%, about 60% to about 100%, about 60% to about 95%, about 60% to about 90%, about 60% to about 85%, about 60% to about 80%, about 60% to about 75%, about 60% to about 70%, about 60% to about 65%, about 65% to about 100%, about 65% to about 95%, about 65% to about 90%, about 65% to about 85%, about 65% to about 80%, about 65% to about 75%, about 65% to about 70%, about 70% to about 100%, about 70% to about 95%, about 70% to about 90%, about 70% to about 85%, about 70% to about 80%, about 70% to about 75%, about 75% to about 100%, about 75% to about 95%, about 75% to about 90%, about 75% to about 85%, about 75% to about 80%, about 80% to about 100%, about 80% to about 95%, about 80% to about 90%, about 80% to about 85%, about 85% to about 100%, about 85% to about 95%, about 85% to about 90%, about 90% to about 100%, about 90% to about 95%, or about 95% to about 100% compared to prior to contacting the cell with the at least one epigenetic modifying agent.

In some embodiments, the site-specific targeting moiety comprises a polymeric molecule.

In some embodiments, the polymeric molecule comprises a polyamide.

In some embodiments, the polymeric molecule comprises a polynucleotide.

In some embodiments, the expression control region comprises a specific transcriptional control element for CXCL9, CXCL10, or CXCL11.

In some embodiments, the epigenetic modifying agent targets at least two specific transcriptional control elements, each of which independently reduces transcription of CXCL9, CXCL10 or CXCL11.

In some embodiments, the transcriptional control element comprises a promoter for CXCL9, CXCL10, or CXCL11.

In some embodiments, the transcriptional control element comprises an enhancer for CXCL9, CXCL10, or CXCL11.

In some embodiments, the epigenetic modifying agent comprises a nucleotide sequence having at least 85%, 90%, 95%, 96%, 97%, 98% or 99% nucleotide identity to the entire nucleotide sequence of any of the nucleotide sequences in any one of Table 12 or Table 13.

In some embodiments, the epigenetic modifying agent comprises a polynucleotide encoding a Cas or dCas polypeptide, a DNA-binding domain of a Transcription activator-like effector (TALE) polypeptide, or a zinc finger (ZFP) polypeptide, or fragment thereof, that specifically targets the expression control region of CXCL9, CXCL10, or CXCL11.

In some embodiments, the DNA-binding domain of the ZFP or TALE comprises an amino acid sequence having at least 85%, 90%, 95%, 96%, 97%, 98% or 99% amino acid identity to the entire amino acid sequence of an amino acid sequence selected from the amino acid sequences listed in Table 3 or Table 5.

In some embodiments, the expression control region comprises 1) one or more CXCL9-associated anchor sequences within an anchor sequence-mediated conjunction comprising a first and a second CXCL9-associated anchor sequence; and/or 2) one or more CXCL10-associated anchor sequences within an anchor sequence-mediated conjunction comprising a first and a second CXCL10-associated anchor sequence; and/or 3) one or more CXCL11-associated anchor sequences within an anchor sequence-mediated conjunction comprising a first and a second CXCL11-associated anchor sequence.

In some embodiments, the anchor sequence comprises a CCCTC-binding factor (CTCF) binding motif.

In some embodiments, the anchor sequence-mediated conjunction comprises one or more transcriptional control elements internal to the conjunction.

In some embodiments, the anchor sequence-mediated conjunction comprises one or more transcriptional control elements external to the conjunction.

In some embodiments, the first and/or the second anchor sequence is located within about 500 kb of the transcriptional control element.

In some embodiments, the first and/or the second anchor sequence is located within about 300 kb of the transcriptional control element.

In some embodiments, the anchor sequence is located within 10 kb of the transcriptional control element.

In some embodiments, the expression control region comprises a specific transcriptional control element for CXCL9, CXCL10, or CXCL11.

In some embodiments, the transcriptional control element comprises a promoter for CXCL9, CXCL10, or CXCL11.

In some embodiments, the transcriptional control element comprises a transcriptional repressor.

In some embodiments, the epigenetic modifying agent comprises a polynucleotide encoding a Cas or dCas polypeptide, a DNA-binding domain, or fragment thereof, of a zinc finger polypeptide (ZFP) that specifically binds to the expression control region of CXCL9, CXCL10, or CXCL11, or a transcription activator-like effector (TALE) polypeptide that specifically binds to the expression control region of CXCL9, CXCL10, or CXCL11.

In some embodiments, the DNA-binding domain of the ZFP comprises an amino acid sequence having at least 85%, 90%, 95%, 96%, 97%, 98% or 99% amino acid identity to the entire amino acid sequence of an amino acid sequence selected from the amino acid sequences listed in Table 3.

In some embodiments, the epigenetic modifying agent comprises a nucleotide modification.

In some embodiments, the polymeric molecule comprises a peptide nucleic acid (PNA).

In some embodiments, the effector molecule comprises a polypeptide.

In some embodiments, the polypeptide comprises a nucleic acid molecule encoding a fusion protein comprising the site-specific targeting moiety which targets an expression regulatory region of CXCL9, CXCL10, or CXCL11, and the effector molecule.

In some embodiments, the fusion protein comprises a peptide-nucleic acid fusion molecule

In some embodiments, the effector is selected from the group consisting of a nuclease, a physical blocker, an epigenetic recruiter, and an epigenetic modifier, and combinations of any of the foregoing.

In some embodiments, the effector is selected from the group consisting of euchromatic histone-lysine N-methyltransferase 2 (G9a), enhancer of zeste homolog 2 (EZH2), MQ1 domain (MQ1), Krüppel associated box (KRAB) transcriptional repression domain, and histone deacetylase 8 (HDAC8).

In some embodiments, the effector is MQ1.

In some embodiments, the effector is KRAB.

In some embodiments, the effector comprises a CRISPR associated protein (Cas) polypeptide or nucleic acid molecule encoding the Cas polypeptide.

In some embodiments, the Cas polypeptide is an enzymatically inactive Cas polypeptide.

In some embodiments, the method further comprises a catalytically active domain of human exonuclease 1 (hEXO1).

In some embodiments, the epigenetic modifier comprises a transcriptional repressor.

In some embodiments, the transcriptional repressor is selected from the group comprising euchromatic histone-lysine N-methyltransferase 2 (G9a), enhancer of zeste homolog 2 (EZH2), MQ1 domain (MQ1), Krüppel associated box (KRAB) transcriptional repression domain, and histone deacetylase 8 (HDAC8).

In some embodiments, the transcriptional repressor is MQ1 domain.

In some embodiments, the MQ1 domain comprises an amino acid sequence having at least about 85%, 90%, 95%, 96%, 97%, 98% or 99% amino acid identity to the entire amino acid sequence of SEQ ID NO: 79.

In some embodiments, the transcriptional repressor comprises two, three, four, or five MQ1s.

In some embodiments, the transcriptional repressor is EZH2.

In some embodiments, the EZH2 has an amino acid sequence having at least about 85%, 90%, 95%, 96%, 97%, 98% or 99% amino acid identity to the entire amino acid sequence of SEQ ID NO: 78.

In some embodiments, the epigenetic modifier comprises a DNA methylase, or a histone deacetylase.

In some embodiments, the effector molecule comprises a Krüppel associated box (KRAB) transcriptional repression domain.

In some embodiments, the KRAB comprises an amino acid sequence having at least about 85%, 90%, 95%, 96%, 97%, 98% or 99% identity to the entire amino acid sequence of SEQ ID NO: 297.

In some embodiments, the effector molecule comprises a Transcription activator-like effector nuclease (TALEN) polypeptide.

In some embodiments, the fusion protein comprises an enzymatically inactive Cas polypeptide and an epigenetic recruiter polypeptide.

In some embodiments, the fusion protein comprises an enzymatically active Cas polypeptide and an epigenetic modifier polypeptide.

In some embodiments, the epigenetic modifying agent comprises a second nucleic acid molecule encoding a second fusion protein, wherein 1) the second fusion protein comprises a second site-specific targeting moiety which targets a second expression control region of CXCL9, CXCL10 or CXCL11, which is the same or different than the target of the first site-specific targeting moiety;

2) the second fusion comprises a second site-specific targeting moiety which targets a second CXCL9 expression control region and a second effector molecule, wherein the second CXCL9 expression control region is different than the CXCL9 expression control region; and/or 3) the second fusion comprises a second site-specific targeting moiety which targets a second CXCL10 expression control region and a second effector molecule, wherein the second CXCL10 expression control region is different than the CXCL10 expression control region; and/or 4) the second fusion comprises a second site-specific targeting moiety which targets a second CXCL11 expression control region and a second effector molecule, wherein the second CXCL10 expression control region is different than the CXCL11 expression control region.

In some embodiments, 1) the epigenetic modifying agent targets a CXCL9 expression control region comprising either a ZF target sequence comprising SEQ ID NOs: 422-425, or TALE target sequence comprising SEQ ID NOs: 429-432, or a combination thereof and, optionally, a second CXCL9 expression control region comprising either a ZF target sequence comprising SEQ ID NOs: 422-425, or TALE target sequence comprising SEQ ID NOs: 429-432, or a combination thereof; and/or 2) the epigenetic modifying agent targets a CXCL10 expression control region comprising either a ZF target sequence comprising SEQ ID NOs: 422-425, or TALE target sequence comprising SEQ ID NOs: 429-432, or a combination thereof and, optionally, a second CXCL10 expression control region comprising either a ZF target sequence comprising SEQ ID NOs: 422-425, or TALE target sequence comprising SEQ ID NOs: 429-432, or a combination thereof; and/or 3) the epigenetic modifying agent targets a CXCL11 expression control region comprising either a ZF target sequence comprising SEQ ID NOs: 422-425, or TALE target sequence comprising SEQ ID NOs: 429-432, or a combination thereof and, optionally, a second CXCL11 expression control region comprising either a ZF target sequence comprising SEQ ID NOs: 422-425, or TALE target sequence comprising SEQ ID NOs: 429-432, or a combination thereof.

In some embodiments, 1) the epigenetic modifying agent targets a CXCL9 expression control region comprising either a ZF target sequence comprising SEQ ID NOs: 422-425, or TALE target sequence comprising SEQ ID NOs: 429-432, or a combination thereof, and a second CXCL9 expression control region comprising either a ZF target sequence comprising SEQ ID NOs: 422-425, or TALE target sequence comprising SEQ ID NOs: 429-432, or a combination thereof; and/or 2) the epigenetic modifying agent targets a CXCL10 expression control region comprising either a ZF target sequence comprising SEQ ID NOs: 422-425, or TALE target sequence comprising SEQ ID NOs: 429-432, or a combination thereof, and a second CXCL10 expression control region comprising either a ZF target sequence comprising SEQ ID NOs: 422-425, or TALE target sequence comprising SEQ ID NOs: 429-432, or a combination thereof; and/or 3) the epigenetic modifying agent targets a CXCL11 expression control region comprising either a ZF target sequence comprising SEQ ID NOs: 422-425, or TALE target sequence comprising SEQ ID NOs: 429-432, or a combination thereof, and a second CXCL11 expression control region comprising either a ZF target sequence comprising SEQ ID NOs: 422-425, or TALE target sequence comprising SEQ ID NOs: 429-432, or a combination thereof.

In some embodiments, the second effector is different than the effector.

In some embodiments, the second effector is the same as the effector.

In some embodiments, the fusion protein and the second fusion protein are operably linked.

In some embodiments, the fusion protein and the second fusion protein independently comprise an amino acid sequence that has at least about 85%, 90%, 95%, 96%, 97%, 98% or 99% sequence identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

In some embodiments, the fusion protein comprises an amino acid sequence having at least 85%, 90%, 95%, 96%, 97%, 98% or 99% sequence identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

In some embodiments, the polypeptide is ZF05-KRAB.

In some embodiments, the polypeptide is ZF07-KRAB.

In some embodiments, the polypeptide is ZF16-KRAB.

In some embodiments, the polypeptide is ZF24-KRAB.

In some embodiments, the polypeptide is ZF05-MQ1.

In some embodiments, the polypeptide is ZF07-MQ1.

In some embodiments, the polypeptide is TAL04-KRAB.

In some embodiments, the polypeptide is TAL08-KRAB.

In some embodiments, the polypeptide is TAL09-KRAB.

In some embodiments, the polypeptide is TAL14-KRAB.

In some embodiments, the fusion protein, and optionally, the second fusion protein, are independently encoded by a polynucleotide comprising a nucleotide sequence that has at least about 85%, 90%, 95%, 96%, 97%, 98% or 99% nucleotide sequence identity to the entire nucleotide sequence of a polynucleotide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

In some embodiments, the administration of the epigenetic modifying agent and the second epigenetic modifying agent has a synergistic effect in reducing the expression of CXCL9, CXCL10, or CXCL11.

In some embodiments, the expression control region comprises a sequence selected from ZF24 target sequence (SEQ ID NO: 422), ZF16 target sequence (SEQ ID NO: 423), ZF07 target sequence (SEQ ID NO: 424), ZF05 target sequence (SEQ ID NO: 425), TAL04 target sequence (SEQ ID NO: 429), TAL08 target sequence (SEQ ID NO: 430), TAL09 target sequence (SEQ ID NO: 431), and TAL14 target sequence (SEQ ID NO: 432).

In some embodiments, the first expression control region comprises a ZF05 target sequence (SEQ ID NO: 425) and the second expression control region comprises a TAL08 target sequence (SEQ ID NO: 430) or TAL14 target sequence (SEQ ID NO: 432).

In some embodiments, the expression control region comprises a ZF05 target sequence (SEQ ID NO: 425).

In some embodiments, the epigenetic modifying agent, the effector, or both the epigenetic modifying agent and the effector are present in a vector.

In some embodiments, the epigenetic modifying agent and the effector are present in the same vector.

In some embodiments, the epigenetic modifying agent and the effector are present in different vectors.

In some embodiments, the vector is a viral expression vector.

In some embodiments, the epigenetic modifying agent, the effector, or both the epigenetic modifying agent and the effector are present in a composition.

In some embodiments, the epigenetic modifying agent and the effector are present in the same composition.

In some embodiments, the epigenetic modifying agent and the effector are present in different compositions.

In some embodiments, the composition comprises a pharmaceutical composition.

In some embodiments, the pharmaceutical composition comprises a lipid formulation.

In some embodiments, the lipid formulation comprises one or more cationic lipids, one or more non-cationic lipids, one or more cholesterol-based lipids, or one or more PEG-modified lipids, or combinations of any of the foregoing.

In some embodiments, the pharmaceutical composition comprises a lipid nanoparticle.

In some embodiments, the cell is a mammalian cell.

In some embodiments, the mammalian cell is a somatic cell.

In some embodiments, the mammalian cell is a primary cell.

In some embodiments, the cell is an immune cell.

In some embodiments, the contacting is performed in vitro.

In some embodiments, the contacting is performed in vivo.

In some embodiments, the contacting is performed ex vivo.

In some embodiments, the method further comprising administering the cell to a subject.

In some embodiments, the cell is within a subject.

In some embodiments, the subject has a disease associated with CXCL9, CXCL10, and/or CXCL11.

In some embodiments, the disease associated with CXCL9, CXCL10, and/or CXCL11 is selected from the group consisting of acute and chronic liver disease, alagille syndrome, autoimmune hepatitis, biliary atresia, cirrhosis, endoscopic retrograde cholangiopancreatography (ERCP), hemochromatosis, hepatitis A, hepatitis B, alopecia areata, multiple sclerosis, chronic pain, and Human T-lymphotropic virus type 1 (HTLV-1)-associated myelopathy/tropical spastic paraparesis (HAM/TSP).

In one aspect, the disclosure provides a method for treating a subject having a disease associated with CXCL9, CXCL10, and/or CXCL11, comprising administering to the subject a therapeutically effective amount of at least one epigenetic modifying agent, each epigenetic modifying agent comprising at least one site-specific targeting moiety which targets a target sequence in an expression control region of human CXCL9, CXCL10, or CXCL11, and an effector molecule, thereby treating the subject.

In one aspect, the disclosure provides a method for decreasing T cell migration in a subject having a disease, comprising administering to the subject a therapeutically effective amount of at least one epigenetic modifying agent, each epigenetic modifying agent comprising at least one site-specific targeting moiety which targets a target sequence in an expression control region of human CXCL9, CXCL10, or CXCL11, and an effector molecule, thereby decreasing T cell migration the subject.

In some embodiments, T cell migration is decreased in the subject is reduced by at least 10%, at least 15%, at least 20%, at least 25%, at least 30%, at least 35%, at least 40%, at least 45%, at least 50%, at least 55%, at least 60%, at least 65%, or at least 70% compared to prior to administering the at least one epigenetic modifying agent to the subject.

In some embodiments, T cell migration is decreased in the subject is reduced by between about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 70%, about 30% to about 65%, about 30% to about 60%, about 30% to about 55%, about 30% to about 50%, about 30% to about 45%, about 30% to about 40%, about 30% to about 35%, about 35% to about 70%, about 35% to about 65%, about 35% to about 60%, about 35% to about 55%, about 35% to about 50%, about 35% to about 45%, about 35% to about 40%, about 40% to about 70%, about 40% to about 65%, about 40% to about 60%, about 40% to about 55%, about 40% to about 50%, about 40% to about 45%, about 45% to about 70%, about 45% to about 65%, about 45% to about 60%, about 45% to about 55%, about 45% to about 50%, about 50% to about 70%, about 50% to about 65%, about 50% to about 60%, about 50% to about 55%, about 55% to about 70%, about 55% to about 65%, about 55% to about 60%, about 60% to about 70%, about 60% to about 65%, or about 65% to about 70% compared to prior to administering the at least one epigenetic modifying agent to the subject.

In some embodiments, the target sequence is within a human chromosomal region selected from the group consisting of chr4: 76928543-76931536, chr4: 76944524-76948668, chr4: 76957098-76962383, chr4: 76949523-76949873, chr4: 76932906-76933929, chr4: 76916900-76917300, chr4: 76988761-76989260, chr4: 77008274-77008727, and chr4: 76957852-76958551.

In some embodiments, the target sequence is located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb from a human chromosomal region selected from the group consisting of chr4: 76958201-76958221, chr4: 76949758-76949778, chr4: 76933245-76933265, chr4: 76932967-76932987, chr4: 76957115-76957132, chr4: 76944734-76944751, chr4: 76944656-76944673, and chr4: 76933040-76933057.

In some embodiments, the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

In some embodiments, the disease associated with CXCL9, CXCL10, and/or CXCL11 is selected from the group consisting of acute and chronic liver disease, alagille syndrome, autoimmune hepatitis, biliary atresia, cirrhosis, endoscopic retrograde cholangiopancreatography (ERCP), hemochromatosis, hepatitis A, hepatitis B, alopecia areata, multiple sclerosis, chronic pain, and Human T-lymphotropic virus type 1 (HTLV-1)-associated myelopathy/tropical spastic paraparesis (HAM/TSP) and the epigenetic modifying agent reduces expression of CXCL9, CXCL10, or CXCL11 in the subject.

In some embodiments, the epigenetic modifying agent and the effector molecule are administered to the subject concurrently.

In some embodiments, the epigenetic modifying agent and the effector molecule are administered to the subject sequentially.

In some embodiments, the effector molecule is administered to the subject prior to administration of the epigenetic modifying agent.

In some embodiments, the epigenetic modifying agent is administered to the subject prior to administration of the effector molecule.

In one aspect, the disclosure provides an epigenetic modifying agent, comprising at least one site-specific targeting moiety which targets a target sequence within an expression control region of human CXCL9, CXCL10, or CXCL11, wherein the target sequence is within a human chromosomal region selected from the group consisting of chr4: 76928543-76931536, chr4: 76944524-76948668, chr4: 76957098-76962383, chr4: 76949523-76949873, chr4: 76932906-76933929, chr4: 76916900-76917300, chr4: 76988761-76989260, chr4: 77008274-77008727, and chr4: 76957852-76958551.

In some embodiments, the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

In some embodiments, the site-specific targeting moiety comprises a nucleotide sequence having at least 85%, 90%, 95%, 96%, 97%, 98% or 99% nucleotide identity to the entire nucleotide sequence of any of the nucleotide sequences in any one of Table 3 or Table 5.

In some embodiments, the site-specific targeting moiety comprises a polymeric molecule comprising a polynucleotide encoding a DNA-binding domain of a Transcription activator-like effector (TALE) polypeptide or a zinc finger (ZFP) polypeptide, or fragment thereof, that specifically binds to the expression control region of CXCL9, CXCL10, or CXCL11.

In some embodiments, the DNA-binding domain of the ZFP or TALE polypeptide comprises an amino acid sequence having at least about 85%, 90%, 95%, 96%, 97%, 98% or 99% amino acid identity to the entire amino acid sequence of any one of the amino acid sequences listed in Table 3 or Table 5.

In some embodiments, the expression control region comprises a nucleotide sequence of a ZF05 target sequence (SEQ ID NO: 407).

In some embodiments, the expression control region comprises a nucleotide sequence of a ZF07 target sequence (SEQ ID NO: 405).

In some embodiments, the expression control region comprises a nucleotide sequence of a ZF16 target sequence (SEQ ID NO: 403).

In some embodiments, the expression control region comprises a nucleotide sequence of a ZF24 target sequence (SEQ ID NO: 401).

In some embodiments, the expression control region comprises a nucleotide sequence of a TAL08 target sequence (SEQ ID NO: 419).

In some embodiments, the expression control region comprises a nucleotide sequence of a TAL14 target sequence (SEQ ID NO: 415).

In some embodiments, the expression control region comprises a nucleotide sequence of a TAL04 target sequence (SEQ ID NO: 421).

In some embodiments, the expression control region comprises a nucleotide sequence of a TAL09 target sequence (SEQ ID NO: 417).

In one aspect, the disclosure provides an epigenetic modifying agent, comprising a polynucleotide encoding the amino acid sequence of ZF05-MQ1 comprising the amino acid sequence of (SEQ ID NO: 466).

In one aspect, the disclosure provides an epigenetic modifying agent, comprising a polynucleotide encoding the amino acid sequence of ZF07-MQ1 comprising the amino acid sequence of (SEQ ID NO: 465).

In one aspect, the disclosure provides an epigenetic modifying agent, comprising a polynucleotide encoding the amino acid sequence of ZF05-KRAB comprising the amino acid sequence of (SEQ ID NO: 467).

In one aspect, the disclosure provides an epigenetic modifying agent, comprising a polynucleotide encoding the amino acid sequence of ZF07-KRAB comprising the amino acid sequence of (SEQ ID NO: 461).

In one aspect, the disclosure provides an epigenetic modifying agent, comprising a polynucleotide encoding the amino acid sequence of ZF24-KRAB comprising the amino acid sequence of (SEQ ID NO: 463).

In one aspect, the disclosure provides an epigenetic modifying agent, comprising a polynucleotide encoding the amino acid sequence of ZF16-KRAB comprising the amino acid sequence of (SEQ ID NO: 464).

In one aspect, the disclosure provides an epigenetic modifying agent, comprising a polynucleotide encoding the amino acid sequence of TAL08-KRAB comprising the amino acid sequence of (SEQ ID NO: 470).

In one aspect, the disclosure provides an epigenetic modifying agent, comprising a polynucleotide encoding the amino acid sequence of a TAL14-KRAB comprising the amino acid sequence of (SEQ ID NO: 468).

In one aspect, the disclosure provides an epigenetic modifying agent, comprising a polynucleotide encoding the amino acid sequence of a TAL04-KRAB comprising the amino acid sequence of (SEQ ID NO: 471).

In one aspect, the disclosure provides an epigenetic modifying agent, comprising a polynucleotide encoding the amino acid sequence of a TAL09-KRAB comprising the amino acid sequence of (SEQ ID NO: 469).

In one aspect, the disclosure provides an epigenetic modifying agent, comprising a polynucleotide encoding the amino acid sequence of a ZF05-KRAB-tPT2A-TAL14-KRAB comprising the amino acid sequence of (SEQ ID NO: 474).

In one aspect, the disclosure provides a vector comprising a nucleic acid molecule encoding the epigenetic modifying agent of the above aspects or any other aspect of the invention delineated herein.

In some embodiments, the vector is a viral expression vector.

In one aspect, the disclosure provides a pharmaceutical composition comprising the epigenetic modifying agent of the above aspects or any other aspect of the invention delineated herein, or the vector of the above aspects or any other aspect of the invention delineated herein.

In some embodiments, the pharmaceutical composition comprises a lipid formulation.

In some embodiments, the lipid formulation comprises one or more cationic lipids, one or more non-cationic lipids, one or more cholesterol-based lipids, or one or more PEG-modified lipids, or combinations of any of the foregoing.

In some embodiments, the pharmaceutical composition comprises a lipid nanoparticle.

In one aspect, the disclosure provides a pharmaceutical composition, comprising an epigenetic modifying agent, comprising a polynucleotide encoding the amino acid sequence of ZF05-MQ1 comprising the amino acid sequence of (SEQ ID NO: 466); and a lipid nanoparticle.

In one aspect, the disclosure provides a pharmaceutical composition, comprising an epigenetic modifying agent, comprising a polynucleotide encoding the amino acid sequence of ZF07-MQ1 comprising the amino acid sequence of (SEQ ID NO: 465); and a lipid nanoparticle.

In one aspect, the disclosure provides a pharmaceutical composition, comprising an epigenetic modifying agent, comprising a polynucleotide encoding the amino acid sequence of a ZF05-KRAB comprising the amino acid sequence of (SEQ ID NO: 467); and a lipid nanoparticle.

In one aspect, the disclosure provides a pharmaceutical composition, comprising an epigenetic modifying agent, comprising a polynucleotide encoding the amino acid sequence of a ZF07-KRAB comprising the amino acid sequence of (SEQ ID NO: 461); and a lipid nanoparticle.

In one aspect, the disclosure provides a pharmaceutical composition, comprising an epigenetic modifying agent, comprising a polynucleotide encoding the amino acid sequence of a ZF24-KRAB comprising the amino acid sequence of (SEQ ID NO: 463); and a lipid nanoparticle.

In one aspect, the disclosure provides a pharmaceutical composition, comprising an epigenetic modifying agent, comprising a polynucleotide encoding the amino acid sequence of a ZF16-KRAB comprising the amino acid sequence of (SEQ ID NO: 464); and a lipid nanoparticle.

In one aspect, the disclosure provides a pharmaceutical composition, comprising an epigenetic modifying agent, comprising a polynucleotide encoding the amino acid sequence of a TAL08-KRAB comprising the amino acid sequence of (SEQ ID NO: 470); and a lipid nanoparticle.

In one aspect, the disclosure provides a pharmaceutical composition, comprising an epigenetic modifying agent, comprising a polynucleotide encoding the amino acid sequence of a TAL14-KRAB comprising the amino acid sequence of (SEQ ID NO: 468); and a lipid nanoparticle.

In one aspect, the disclosure provides a pharmaceutical composition, comprising an epigenetic modifying agent, comprising a polynucleotide encoding the amino acid sequence of a TAL04-KRAB comprising the amino acid sequence of (SEQ ID NO: 471); and a lipid nanoparticle.

In one aspect, the disclosure provides a pharmaceutical composition, comprising an epigenetic modifying agent, comprising a polynucleotide encoding the amino acid sequence of a TAL09-KRAB comprising the amino acid sequence of (SEQ ID NO: 469); and a lipid nanoparticle.

In one aspect, the disclosure provides a pharmaceutical composition, comprising an epigenetic modifying agent, comprising a polynucleotide encoding the amino acid sequence of a ZF05-KRAB-tPT2A-TAL14-KRAB comprising the amino acid sequence of (SEQ ID NO: 474); and a lipid nanoparticle.

In one aspect, the disclosure provides a cell comprising the epigenetic modifying agent of the above aspects or any other aspect of the invention delineated herein, or the vector of the above aspects or any other aspect of the invention delineated herein.

In one aspect, the disclosure provides a method of reducing expression of CXCL9, CXCL10, or CXCL11 in a cell, the method comprising contacting the cell with an epigenetic modifying agent of the above aspects or any other aspect of the invention delineated herein, or the vector of the above aspects or any other aspect of the invention delineated herein, or the pharmaceutical composition of the above aspects or any other aspect of the invention delineated herein.

In some embodiments, the expression of CXCL9, CXCL10, or CXCL11 in the cell is reduced by at least 30%, at least 35%, at least 40%, at least 45%, at least 50%, at least 55%, at least 60%, at least 65%, at least 70%, at least 75%, at least 80%, at least 85%, at least 90%, or at least 95% compared to prior to contacting the cell with the at least one epigenetic modifying agent.

In some embodiments, the expression of CXCL9, CXCL10, or CXCL11 in the cell is reduced between about 30% to about 100%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 35% to about 100%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 40% to about 100%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 100%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 100%, about 55% to about 95%, about 55% to about 90%, about 55% to about 85%, about 55% to about 80%, about 55% to about 75%, about 55% to about 70%, about 55% to about 65%, about 55% to about 60%, about 60% to about 100%, about 60% to about 95%, about 60% to about 90%, about 60% to about 85%, about 60% to about 80%, about 60% to about 75%, about 60% to about 70%, about 60% to about 65%, about 65% to about 100%, about 65% to about 95%, about 65% to about 90%, about 65% to about 85%, about 65% to about 80%, about 65% to about 75%, about 65% to about 70%, about 70% to about 100%, about 70% to about 95%, about 70% to about 90%, about 70% to about 85%, about 70% to about 80%, about 70% to about 75%, about 75% to about 100%, about 75% to about 95%, about 75% to about 90%, about 75% to about 85%, about 75% to about 80%, about 80% to about 100%, about 80% to about 95%, about 80% to about 90%, about 80% to about 85%, about 85% to about 100%, about 85% to about 95%, about 85% to about 90%, about 90% to about 100%, about 90% to about 95%, or about 95% to about 100% compared to prior to contacting the cell with the at least one epigenetic modifying agent.

### BRIEF DESCRIPTION OF THE DRAWINGS

FIGS. 1A-1C. Treatment with combinations of OECs reduce CXCL9-10-11 in AML-12 murine hepatic cell line. Cells were transfected with 0.6 ug/ml OEC (single treatments) or a total of 2 μg/ml OECs (combination treatments). TAL37-KRAB reduced CXCL9 expression (80%); TAL34-KRAB reduced CXCL10 expression (60%); TAL27-KRAB reduced CXCL11 expression (75%); Triple combination of OECs reduced CXC9-10-11 expression (75-90%). FIG. 1A: CXCL9; FIG. 1B: CXCL10; and FIG. 1C: CXCL11.

FIGS. 2A-2C. Treatment with human monocistronic OECs reduces CXCL9/10/11 mRNA levels in primary human hepatocytes. Treatment with 10 different monocistronic human OECs identified multiple OECs that reduce mRNA expression of FIG. 2A: CXCL9 (>90%), FIG. 2B: CXCL10 (>90%), and FIG. 2C: CXCL11 (>95%).

FIGS. 3A-3C. Treatment with human monocistronic OECs reduce CXCL9-10-11 protein in conditioned primary human hepatocytes media. ZF05-KRAB reduced CXCL9 expression (70%), CXCL10 expression (50%), and CXCL11 expression (50%). TAL14-KRAB reduced CXCL10 expression (20%) and CXCL11 expression (95%). FIG. 3A: CXCL9; FIG. 3B: CXCL10; and FIG. 3C: CXCL11.

FIGS. 4A-4C. Treatment with combinations of human OECs reduce CXCL9-10-11 in primary human hepatocytes. The monocistronic combination ZF05-KRAB+TAL14-KRAB reduced CXCL9 expression (80%), CXCL10 expression (60%), and CXCL11 expression (95%). The bicistronic ZF05-KRAB_TAL14-KRAB reduced CXCL9 expression (70%), CXCL10 expression (50%), and CXCL11 expression (85%). FIG. 4A: CXCL9; FIG. 4B: CXCL10; and FIG. 4C: CXCL11.

FIGS. 5A and 5B. Hepatocyte treatment with human monocistronic OECs significantly reduces T cell migration. FIG. 5A Conditioned media from IFNG-stimulated, OEC-treated hepatocytes was used in a T cell chemotaxis assay. Combination treatments of ZF05-KRAB with either TAL14-KRAB or TAL08-KRAB resulted in ˜35% reduction in T cell migration. FIG. 5B Conditioned media from IFNG-stimulated, OEC-treated hepatocytes was used in a T cell chemotaxis assay. Combination treatments of ZF05-KRAB with TAL14-KRAB resulted in more than 50% reduction in T cell migration. *1-way ANOVA p<0.05.

### DETAILED DESCRIPTION OF THE INVENTION

The present invention provides agents and compositions for reducing the expression (e.g., reducing expression) of a CXCL9, CXCL10, and/or CXCL11 gene by targeting a CXCL9, CXCL10, and/or CXCL11 expression control region. The CXCL9, CXCL10, and/or CXCL11 gene may be in a cell, e.g., a mammalian cell, such as a mammalian somatic cell, e.g., a human somatic cell. The present invention also provides methods of using the agents and compositions of the invention for reducing the expression of a CXCL9, CXCL10, and/or CXCL11 gene or for treating a subject who would benefit from reducing the expression of a CXCL9, CXCL10, and/or CXCL11 gene, e.g., a subject suffering or prone to suffering from a CXCL9, CXCL10, and/or CXCL11-associated disease. e.g., liver disease, e.g., inflammatory liver disease.

The agents of the invention are referenced to herein as epigenetic modifying agents and are described in Section II, below.

### Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

As used herein, the terms “C—X—C motif chemokine ligand” and “CXCL”, as used interchangeably herein, are small peptides that are chemokines involved in inflammation and antitumor immunity. CXC refers to the location of the two cysteine residues near the N-terminal, with the X representing any amino acid (cysteine-containing motif). Specifically, chemokines CXCL9, CXCL10, and CXCL11 are expressed and released from hepatoctyes after interferon gamma (IFNG) expression due to inflammation, e.g., inflammation in the liver. CXCL9-10-11 bind CXCR3 on T cells and promote their recruitment to the site of inflammation, e.g., in the liver, resulting in chronic injury. Increase expression and release of CXCL9/CXCL10/CXCL11 can be associated with acute and chronic liver disease, alagille syndrome, autoimmune hepatitis, biliary atresia, cirrhosis, ERCP, hemochromatosis, hepatitis A, hepatitis B, alopecia areata, multiple sclerosis, chronic pain, and Human T-lymphotropic virus type 1 (HTLV-1)-associated myelopathy/tropical spastic paraparesis (HAM/TSP).

CXCL9 is also known as CMK, Humig, MIG, crg-10, SCYB9, monokine induced by gamma interferon, C—X—C motif chemokine 9, monokine induced by interferon-gamma, small-inducible cytokine B9, gamma-interferon-induced monokine, and chemokine (C—X—C motif) ligand. The nucleotide and amino acid sequence of CXCL9, is known and may be found in, for example, GenBank Accession Nos. NM_002416 (NM_002416.3) (mRNA) and NP_002407 (NP_002407.1), the entire contents of each of which are incorporated herein by reference. The nucleotide sequence of the genomic region of Chromosome 4 which includes the endogenous promoters of CXCL9 and the CXCL9 coding sequence is also known and may be found in GenBank Accession No. NC_000004.12 (76001275 . . . 76007509) and NC_060928.1 (79341778 . . . 79348012).

CXCL10 is also known as C7, crg-2, IF110, mob-1, INP10, SCYB10, 10 kDa interferon gamma-induced protein (Gamma-IP10; IP-10), and Small-inducible cytokine B10. The nucleotide and amino acid sequence of CXCL10, is known and may be found in, for example, GenBank Accession Nos. NM_001565 (NM_001565.4) (mRNA), NR_168520.1 (RNA) and NP_001556 (NP_001556.2), the entire contents of each of which are incorporated herein by reference. The nucleotide sequence of the genomic region of Chromosome 4 which includes the endogenous promoters of CXCL10 and the CXCL10 coding sequence is also known and may be found in GenBank Accession No. NC_000004.12 (76021118 . . . 76023497) and NC_060928.1 (79361779 . . . 79364158).

CXCL11 is also known as H174, I-TAC, IP-9, IP9, SCYB11, SCYB9B, b-R1. The nucleotide and amino acid sequence of CXCL11, is known and may be found in, for example, GenBank Accession Nos. NM_001302123 (NM_001302123.2) (mRNA) and NP_001289052 (NP_001289052.1) refer to CXCL11 isoform 2 precursor, and NM_005409 (NM_005409.5) (mRNA) and NP_005400 (NP_005400.1) refer to CXCL11 isoform 1 precursor, the entire contents of each of which are incorporated herein by reference. The nucleotide sequence of the genomic region of Chromosome 4 which includes the endogenous promoters of CXCL11 and the CXCL11 coding sequence is also known and may be found in GenBank Accession No. NC 000004.12 (76033682 . . . 76036070) and NC_060928.1 (79374343 . . . 79376731).

The term “site-specific epigenetic modifying agent” or “epigenetic modifying agent”, used herein interchangeably, refers to any agent that specifically binds to a target expression control region of CXCL9, CXCL10, and/or CXCL11 and reduces expression, e.g., of a CXCL9, CXCL10, and/or CXCL11 gene. The reduction of expression may be permanent or transient. Epigenetic modifying agents of the invention may comprise a “site-specific targeting moiety.”

As used herein, the term “site-specific targeting moiety” refers to a moiety that specifically binds to an expression control region of CXCL9, CXCL10, and/or CXCL11, e.g., a transcriptional control region of a CXCL9, CXCL10, and/or CXCL11 gene, such as a DNA region around/proximally upstream of the transcription start site, a promoter, an enhancer, or a repressor; or an anchor sequence associated with a CXCL9, CXCL10, and/or CXCL11 gene, such as, for example within an anchor sequence-mediated conjunction associated with a CXCL9, CXCL10, and/or CXCL11 gene. Exemplary “site-specific targeting moieties” include, but are not limited to, polyamides, nucleic acid molecules, such as RNA, DNA, or modified RNA or DNA, polypeptides, protein nucleic acid molecules, and fusion proteins.

As used herein, the terms “specific binding” or “specifically binds” refer to an ability to discriminate between possible binding partners in the environment in which binding is to occur. In some embodiments, an epigenetic modifying agent that interacts, e.g., preferentially interacts, with one particular target when other potential epigenetic modifying agents are present is said to “bind specifically” to the target (i.e., the expression control region) with which it interacts. In some embodiments, specific binding is assessed by detecting or determining the degree of association between the epigenetic modifying agent and its target; in some embodiments, specific binding is assessed by detecting or determining degree of dissociation of an epigenetic modifying agent-target complex. In some embodiments, specific binding is assessed by detecting or determining ability of the epigenetic modifying agent to compete with an alternative interaction between its target and another entity. In some embodiments, specific binding is assessed by performing such detections or determinations across a range of concentrations.

As used herein, the term “expression control region” or expression control domain′ refers to a region or domain present in a genomic DNA that modulates the expression of a target gene in a cell. A functionality associated with an expression control region may directly affect expression of a target gene, e.g., by recruiting or blocking recruitment of a transcription factor that would stimulate expression of the gene. A functionality associated with an expression control region may indirectly affect expression of a target gene, e.g., by introducing epigenetic modifications or recruiting other factors that introduce epigenetic modifications that induce a change in chromosomal topology that modulates expression of a target gene. Expression control regions may be upstream and/or downstream of the protein coding sequence of a gene and include, for example, transcriptional control elements, e.g., DNA regions around/proximally upstream of the transcription start site, promoters, enhancers, or repressors; and anchor sequences, and anchor sequence-mediated conjunctions.

The term “transcriptional control element,” as used herein, refers to a nucleic acid sequence that controls transcription of a gene. Transcriptional control elements include, for example, anchor sequences, anchor sequence-mediated conjunctions, DNA regions around/proximally upstream of the transcription start site, promoters, enhancers, promoters, transcriptional enhancers, and transcriptional repressors.

A transcription start site (TSS) is the location where transcription starts at the 5′-end of a gene sequence. The DNA regions around/proximally upstream of the TSS can regulate the expression of a gene by, for example, recruiting a transcription factor. Alteration in the modification status of one or more nucleotides (e.g., methylation) or one or more chromatin proteins (e.g., acetylation) in the DNA regions around/proximally upstream of the TSS can regulate the expression of a gene.

A promoter is a region of DNA recognized by an RNA polymerase to initiate transcription of a particular gene and is generally located upstream of the 5′-end of the transcription start site of the gene.

A “transcriptional enhancer” increases gene transcription. A “transcriptional silencer” or “transcriptional repressor” decreases gene transcription. Enhancing and silencing sequences may be about 50-3500 base pairs in length and may influence gene transcription up to about 1 megabases away.

The term “gene,” as used herein, refers to a sequence of nucleotides that encode a molecule, such as a protein, that has a function. A gene contains sequences that are transcribed (e.g., a 3′UTR), sequences that are not transcribed (e.g., a promoter), sequences that are translated (e.g., an exon), and sequences that are not translated (e.g., intron).

As used herein, the term “target gene” means a CXCL9, CXCL10, and/or CXCL11 gene that is targeted for decrease of expression. In some embodiments, a CXCL9, CXCL10, and/or CXCL11 target gene is part of a targeted genomic complex (e.g. a CXCL9, CXCL10, and/or CXCL11 gene that has at least part of its genomic sequence as part of a target genomic complex, e.g. inside an anchor sequence-mediated conjunction), which genomic complex is targeted by one or more site-specific epigenetic modifying agents as described herein. In some embodiments, the target gene is targeted for inhibition of expression. In some embodiments, expression of a CXCL9, CXCL10, and/or CXCL11 gene is decreased by contacting the CXCL9, CXCL10, and/or CXCL11 gene or a transcription control element operably linked to the CXCL9, CXCL10, and/or CXCL11 gene with one or more site-specific epigenetic modifying agents as described herein. In some embodiments, a CXCL9, CXCL10, and/or CXCL11 gene is aberrantly expressed (e.g., over-expressed) in a cell, e.g., a cell in a subject (e.g., a subject having a disease or auto-immune disease associated with CXCL9, CXCL10, and/or CXCL11).

The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present invention, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule, e.g., the same chromosome) as the other targeted anchor sequence is not modulated. In some embodiments, the anchor sequence comprises or is a nucleating polypeptide binding motif. In some embodiments, the anchor sequence is adjacent to a nucleating polypeptide binding motif.

The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences.

As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex bind. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory region (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that the stretch of genomic DNA does not adopt when the complex is not formed.

An “effector molecule,” as used herein, refers to a molecule that is able to regulate a biological activity, such as enzymatic activity, gene expression, anchor sequence-mediated conjunction or cell signaling. Exemplary effectors are described in Section II, below, and in some embodiment include, for example, nucleases, physical blockers, epigenetic recruiters, e.g., a transcriptional repressor and epigenetic CpG modifiers, e.g., a DNA methylase, a histone modifying agent, or a histone deacetylase, and combinations of any of the foregoing.

### Site-Specific Epigenetic Modifying Agent of the Invention

The present invention provides site-specific epigenetic modifying agents which, in one aspect of the invention include a site-specific targeting moiety which targets a CXCL9, CXCL10, and/or CXCL11 expression control region. In another aspect, the site-specific epigenetic modifying agents of the invention include a site-specific targeting moiety which targets a CXCL9, CXCL10, and/or CXCL11 expression control region, and an effector molecule. As will be appreciated by one of ordinary skill in the art, such epigenetic modifying agents are site-specific and, thus, specifically bind to a CXCL9, CXCL10, and/or CXCL11 expression control region (e.g., one or more transcriptional control elements and/or one or more target anchor sequences), e.g., within a cell and not to non-targeted expression control regions (e.g., within the same cell).

CXCL9, CXCL10, and CXCL11 are chemokines which are expressed and released from hepatocytes after stimulation by IFN gamma resulting CXCL9-10-11 binding to CXCR3 on T cells and promoting recruitment to the site of inflammation. The present invention features the use of effector molecules, e.g., chromatin remodelers, that when fused to DNA-targeting moieties can induce epigenetic changes at specific genomic regions that lead to decreased transcription of targeted genes, e.g., CXCL9, CXCL10, and/or CXCL11 gene. In certain embodiments, an effector molecule, HDAC8, G9A, EZH2, KRAB, or MQ1, fused to dCas9, a zinc finger domain (ZF), or a TALE domain, which is the DNA targeting moiety, is targeted to the CXCL9, CXCL10, and/or CXCL11 locus. Cas9-fused epigenetic modifying agents use single guide RNAs (sgRNAs) complementary to the DNA region around/just upstream of the transcription start site (TSS) of a CXCL9, CXCL10, and/or CXCL11 gene to target the epigenetic modifying agent and provoke changes in histone modifications, e.g., methylation. ZF or TALE-fused epigenetic modifying agent are targeted to CXCL9, CXCL10, and/or CXCL11 vis binding of the ZF or TALe domain to the DNA region around/just upstream of the transcription start site (TSS) of a CXCL9, CXCL10, and/or CXCL11 gene.

The present invention features methods to directly target a CXCL9, CXCL10, and CXCL11 gene using targeting moieties (e.g., dCas9, TALEs, or ZF proteins).

The site-specific epigenetic modifying agents of the invention comprise a site-specific targeting moiety targeting a CXCL9, CXCL10, and/or CXCL11 expression control region. The expression control region targeted by the site-specific targeting moiety may be, for example, a transcriptional control element or an anchor sequence, such as an anchor sequence within an anchor-mediated conjunction.

Thus, site-specific epigenetic modifying agents of the invention may reduce expression of a gene, i.e., a CXCL9, CXCL10, and/or CXCL11 gene, e.g., by reducing expression of the CXCL9, CXCL10, and/or CXCL11 gene from a DNA region around/proximally upstream of a transcription start site, an endogenous promoter, an enhancer, or an repressor; may alter methylation of the control region; may alter acetylation of the chromatin protein; may introduce one or more mutations, e.g., substitution, addition or deletion of nucleotide; may alter at least one anchor sequence; may alter at least one conjunction nucleating molecule binding site, such as by altering binding affinity for the conjunction nucleating molecule; may alter an orientation of at least one common nucleotide sequence, such as a CTCF binding motif by, e.g., substitution, addition or deletion in at least one anchor sequence, such as a CTCF binding motif.

In certain embodiments, the site-specific epigenetic modifying agents and compositions described herein target an expression control region comprising one or more transcriptional control elements specific to CXCL9, CXCL10, and/or CXCL11 to reduce expression in a cell. Transcriptional control elements specific to CXCL9, CXCL10, and/or CXCL11 that can be targeted include promoters specific to CXCL9, CXCL10, and/or CXCL11, enhancers specific to CXCL9, CXCL10, and/or CXCL11, and repressors specific to CXCL9, CXCL10, and/or CXCL11. In one embodiment, a promoter specific to specific to CXCL9, CXCL10, or CXCL11 drives expression in cells including, but not limited to, the liver cells, skin cells, heart cells, pancreas cells, pituitary gland cells, and connective tissue cells (e.g., tissues and cells found in joints) e.g., DNA region around or proximally upstream of transcription start site of CXCL9, CXCL10, and/or CXCL11.

For example, a site-specific epigenetic modifying agents may include a site-specific targeting moiety, e.g., a nucleic acid molecule encoding a DNA-binding domain of a Transcription activator-like effector (TALE) polypeptide or a zinc finger (ZFP) polypeptide, or fragment thereof, that specifically targets and binds to an expression control region specific to CXCL9, CXCL10, and/or CXCL11, such as an endogenous promoter region of CXCL9, CXCL10, and/or CXCL11, and an effector molecule, such as an effector molecule that includes a transcriptional repressor that reduces expression of a target gene from an endogenous promoter to reduce gene expression. In one embodiment, the epigenetic modifying agent is “bicistronic nucleic acid molecule,” i.e., capable of making two fusion proteins from a single messenger RNA molecule, a first and a second site-specific targeting moiety, e.g., a nucleic acid molecule encoding a DNA-binding domain of a Transcription activator-like effector (TALE) polypeptide or a zinc finger (ZFP) polypeptide, or fragment thereof, that specifically targets and binds to an expression control region specific to CXCL9, CXCL10, and/or CXCL11, such as an endogenous promoter region specific to CXCL9, CXCL10, and/or CXCL11, and an effector molecule, such as an effector molecule that includes a transcriptional repressor that reduces expression of a target gene from an endogenous promoter to reduce gene expression. In some embodiments of the invention, a site-specific epigenetic modifying agent may include a site-specific targeting moiety, e.g., a nucleic acid molecule, such as a guide RNA targeting an endogenous DNA region around or proximally upstream of CXCL9, CXCL10, and/or CXCL11 transcription starting site, and an effector molecule, such as an effector molecule that includes a transcriptional repressor that reduces expression of a target gene from an endogenous promoter to reduce gene expression.

In certain embodiments of the invention, the site-specific epigenetic modifying agents and compositions described herein target an expression control region comprising one or more anchor sequences associated with CXCL9, CXCL10, and/or CXCL11, e.g., within an anchor sequence-mediated conjunction, comprising a first and a second anchor sequence associated with CXCL9, CXCL10, and/or CXCL11 to alter a two-dimensional chromatin structure, e.g., anchor sequence-mediated conjunctions in order to modulate expression in a cell, e.g., a cell within a subject, e.g., by modifying anchor sequence-mediated conjunctions in DNA, e.g., genomic DNA.

In one aspect, the invention includes a site-specific epigenetic modifying agent comprising a site-specific targeting moiety which targets an expression control region associated with CXCL9, CXCL10, and/or CXCL11 comprising one or more anchor sequences associated with CXCL9, CXCL10, and/or CXCL11 within an anchor sequence-mediated conjunction. The epigenetic modifying agent binds, e.g., specifically binds, a specific anchor sequence-mediated conjunction to alter a topology of the anchor sequence-mediated conjunction, e.g., an anchor sequence-mediated conjunction having a physical interaction of two or more DNA loci bound by a conjunction nucleating molecule.

The formation of an anchor sequence-mediated conjunction may force transcriptional control elements to interact with a CXCL9, CXCL10, and/or CXCL11 gene or spatially constrain the activity of the transcriptional control elements. Altering anchor sequence-mediated conjunctions, therefore, allows for reducing CXCL9, CXCL10, and/or CXCL11 expression without altering the coding sequences of the CXCL9, CXCL10, and/or CXCL11 gene being reduced.

In some embodiments, the site-specific epigenetic modifying agent and compositions of the invention reduce expression of a CXCL9, CXCL10, and/or CXCL11 gene associated with an anchor sequence-mediated conjunction by physically interfering between one or more anchor sequences and a conjunction nucleating molecule. For example, a DNA binding small molecule (e.g., minor or major groove binders), peptide (e.g., zinc finger, TALE, novel or modified peptide), protein (e.g., CTCF, modified CTCF with impaired CTCF binding and/or cohesion binding affinity), or nucleic acids (e.g., ssDNA, modified DNA or RNA, peptide oligonucleotide conjugates, locked nucleic acids, bridged nucleic acids, polyamides, and/or triplex forming oligonucleotides) may physically prevent a conjunction nucleating molecule from interacting with one or more anchor sequences to reduce CXCL9, CXCL10, and/or CXCL11 gene expression.

In some embodiments, the site-specific epigenetic modifying agents and compositions of the invention reduce expression of a CXCL9, CXCL10, and/or CXCL11 gene associated with an anchor sequence-mediated conjunction by modification of an anchor sequence, e.g., epigenetic modifications, e.g., histone protein modifications, or genomic editing modifications. For example, one or more anchor sequences associated with an anchor sequence-mediated conjunction comprising a CXCL9, CXCL10, and/or CXCL11 gene may be targeted for methylation modification by a DNA methyltransferase, e.g., dCas9-methyltransferase fusion, e.g., antisense oligonucleotide-enzyme fusion, to reduce expression of the gene. In another example, one or more anchor sequences associated with an anchor sequence-mediated conjunction comprising a CXCL9, CXCL10, and/or CXCL11 gene may be targeted for genome editing, e.g., Cas9-mediated genome editing.

In some embodiments, the site-specific epigenetic modifying agents and compositions of the invention reduce expression of a CXCL9, CXCL10, and/or CXCL11 gene associated with an anchor sequence-mediated conjunction, e.g., represses transcription, e.g., induces epigenetic changes to chromatin or genome editing.

In some embodiments, an anchor sequence-mediated conjunction includes one or more anchor sequences, a CXCL9, CXCL10, and/or CXCL11 gene, and one or more transcriptional control elements, such as an enhancing or silencing element. In some embodiments, the transcriptional control element is within, partially within, or outside the anchor sequence-mediated conjunction.

In one embodiment, the anchor sequence-mediated conjunction comprises a loop, such as an intra-chromosomal loop. In certain embodiments, the anchor sequence-mediated conjunction has a plurality of loops. One or more loops may include a first anchor sequence, a nucleic acid sequence, a transcriptional control element, and a second anchor sequence. In another embodiment, at least one loop includes, in order, a first anchor sequence, a transcriptional control element, and a second anchor sequence; or a first anchor sequence, a nucleic acid sequence, and a second anchor sequence. In yet another embodiment, either one or both of the nucleic acid sequences and the transcriptional control element is located within or outside the loop. In still another embodiment, one or more of the loops comprises a transcriptional control element.

In some embodiments, the anchor sequence-mediated conjunction includes a TATA box, a CAAT box, a GC box, or a CAP site.

In some embodiments, the anchor sequence-mediated conjunction comprises a plurality of loops, and where the anchor sequence-mediated conjunction comprises at least one of an anchor sequence, a nucleic acid sequence, and a transcriptional control element in one or more of the loops.

In one aspect, the site-specific epigenetic modifying agents and compositions of the invention may introduce a targeted alteration to an anchor sequence-mediated conjunction to modulate expression of a nucleic acid sequence with an epigenetic modifying agent that binds the anchor sequence. In some embodiments, the anchor sequence-mediated conjunction is altered by targeting one or more nucleotides within the anchor sequence-mediated conjunction for substitution, addition or deletion.

In some embodiments, expression, e.g., transcription, is repressed by inclusion of a repressive loop or exclusion of an activating loop. In one such embodiment, the anchor sequence-mediated conjunction includes a transcriptional control element that decreases expression, e.g., transcription, of a nucleic acid sequence, e.g., such as a CXCL9, CXCL10, and/or CXCL11 encoding nucleic acid sequence. In another such embodiment, the anchor sequence-mediated conjunction excludes a transcriptional control sequence that increases transcription of a nucleic acid sequence, e.g., such a CXCL9, CXCL10, and/or CXCL11 encoding nucleic acid.

Each anchor sequence-mediated conjunction comprises one or more anchor sequences, e.g., a plurality. Anchor sequences can be manipulated or altered to disrupt naturally occurring loops or form new loops (e.g., to form exogenous loops or to form non-naturally occurring loops with exogenous or altered anchor sequences). Such alterations modulate CXCL9, CXCL10, and/or CXCL11 gene expression by changing the 2-dimensional structure of DNA containing all or a portion of a CXCL9, CXCL10, and/or CXCL11 gene, e.g., by thereby modulating the ability of the a CXCL9, CXCL10, and/or CXCL11 gene to interact with transcriptional control elements (e.g., enhancing and silencing/repressive sequences). In some embodiments, the chromatin structure is modified by substituting, adding or deleting one or more nucleotides within an anchor sequence of the anchor sequence-mediated conjunction.

The anchor sequences may be non-contiguous with one another. In embodiments with noncontiguous anchor sequences, the first anchor sequence may be separated from the second anchor sequence by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, the first anchor sequence is separated from the second anchor sequence by about 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

In one embodiment, the anchor sequence comprises a common nucleotide sequence, e.g., a CTCF-binding motif:

N (T/C/G) N (G/A/T) CC (A/T/G) (C/G) (C/T/A) AG (G/A) (G/T) GG (C/A/T) (G/A) (C/G) (C/T/A) (G/A/C), where N is any nucleotide.

A CTCF-binding motif may also be in the opposite orientation, e.g.,

(G/A/C)(C/T/A)(C/G)(G/A)(C/A/T)GG(G/T)(G/A)GA
(C/T/A)(C/G)(A/T/G)CC(G/A/T)N(T/C/G)N.

In one embodiment, the anchor sequence comprises

N(T/C/G)N(G/A/T)CC(A/T/G)(C/G)(C/T/A)AG(G/A)(G/T)
GG(C/A/T)(G/A)(C/G)(C/T/A)(G/A/C)
or
(G/A/C)(C/T/A)(C/G)(G/A)(C/A/T)GG(G/T)(G/A)GA
(C/T/A)(C/G)(A/T/G)CC(G/A/T)N(T/C/G)N

or a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to either

N(T/C/G)N(G/A/T)CC(A/T/G)(C/G)(C/T/A)AG(G/A)(G/T)
GG(C/A/T)(G/A)(C/G)(C/T/A)(G/A/C)
or
(G/A/C)(C/T/A)(C/G)(G/A)(C/A/T)GG(G/T)(G/A)GA
(C/T/A)(C/G)(A/T/G)CC(G/A/T)N(T/C/G)N.

In some embodiments, the anchor sequence-mediated conjunction comprises at least a first anchor sequence and a second anchor sequence. The first anchor sequence and second anchor sequence may each comprise a common nucleotide sequence, e.g., each comprises a CTCF binding motif. In some embodiments, the first anchor sequence and second anchor sequence comprise different sequences, e.g., the first anchor sequence comprises a CTCF binding motif and the second anchor sequence comprises an anchor sequence other than a CTCF binding motif. In some embodiments, each anchor sequence comprises a common nucleotide sequence and one or more flanking nucleotides on one or both sides of the common nucleotide sequence.

Two CTCF-binding motifs (e.g., contiguous or non-contiguous CTCF binding motifs) that can form a conjunction may be present in the genome in any orientation, e.g., in the same orientation (tandem) either 5′→3′ (left tandem, e.g., the two CTCF-binding motifs that comprise

N(T/C/G)N(G/A/T)CC(A/T/G)(C/G)(C/T/A)AG(G/A)(G/T)
GG(C/A/T)(G/A)(C/G)(C/T/A)(G/A/C))

or 3′→5′ (right tandem, e.g., the two CTCF-binding motifs comprise

(G/A/C)(C/T/A)(C/G)(G/A)(C/A/T)GG(G/T)(G/A)GA
(C/T/A)(C/G)(A/T/G)CC(G/A/T)N(T/C/G)N),

or convergent orientation, where one CTCF-binding motif comprises

N(T/C/G)N(G/A/T)CC(A/T/G)(C/G)(C/T/A)AG(G/A)(G/T)
GG(C/A/T)(G/A)(C/G)(C/T/A)(G/A/C)

and the other comprises SEQ ID NO: 65. CTCFBSDB 2.0: Database For CTCF binding motifs And Genome Organization (www.insulatordb.uthsc.edu/) can be used to identify CTCF binding motifs associated with a target gene, e.g., CXCL9, CXCL10, and/or CXCL11.

In some embodiments, the anchor sequence-mediated conjunction is altered by changing an orientation of at least one common nucleotide sequence, e.g., a conjunction nucleating molecule binding site.

In some embodiments, the anchor sequence comprises a conjunction nucleating molecule binding site, e.g., CTCF binding motif, and site-specific epigenetic modifying agent of the invention introduces an alteration in at least one conjunction nucleating molecule binding site, e.g. altering binding affinity for the conjunction nucleating molecule.

In some embodiments, the anchor sequence-mediated conjunction is altered by introducing an exogenous anchor sequence. Addition of a non-naturally occurring or exogenous anchor sequence to form or disrupt a naturally occurring anchor sequence-mediated conjunction, e.g., by inducing a non-naturally occurring loop to form that alters transcription of the nucleic acid sequence.

In some embodiments, the anchor sequence-mediated conjunction comprises a CXCL9, CXCL10, and/or CXCL11 gene, and one or more, e.g., 2, 3, 4, 5, or other genes other than the CXCL9, CXCL10, and/or CXCL11 gene.

In some embodiments, the anchor sequence-mediated conjunction is associated with one or more, e.g., 2, 3, 4, 5, or more, transcriptional control elements. In some embodiments, the CXCL9, CXCL10, and/or CXCL11 gene is noncontiguous with one or more of the transcriptional control elements. In some embodiments where the CXCL9, CXCL10, and/or CXCL11 gene is non-contiguous with the transcriptional control element, the gene may be separated from one or more transcriptional control elements by about 100 bp to about 500 Mb, about 500 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, the gene is separated from the transcriptional control element by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

In some embodiments, the type of anchor sequence-mediated conjunction may help to determine how to reduce gene expression, e.g., choice of site-specific targeting moiety, by altering the anchor sequence-mediated conjunction. For example, some types of anchor sequence-mediated conjunctions comprise one or more transcription control elements within the anchor sequence-mediated conjunction. Disruption of such an anchor sequence-mediated conjunction by disrupting the formation of the anchor sequence-mediated conjunction, e.g., altering one or more anchor sequences, is likely to decrease transcription of a CXCL9, CXCL10, and/or CXCL11 gene within the anchor sequence-mediated conjunction.

In some embodiments, expression of the CXCL9, CXCL10, and/or CXCL11 gene is regulated, modulated, or influenced by one or more transcriptional control elements associated with the anchor sequence-mediated conjunction. In some embodiments, the anchor sequence-mediated conjunction comprises a CXCL9, CXCL10, and/or CXCL11 gene and one or more transcriptional control elements. For example, the CXCL9, CXCL10, and/or CXCL11 gene and one or more transcriptional control sequences are located within, at least partially, an anchor sequence-mediated conjunction, e.g., a Type 1 anchor sequence-mediated conjunction. The anchor sequence-mediated conjunction may also be referred to as a “Type 1, EP subtype.” In some embodiments, the CXCL9, CXCL10, and/or CXCL11 gene has a defined state of expression, e.g., in its native state, e.g., in a diseased state. For example, the CXCL9, CXCL10, and/or CXCL11 gene may have a high level of expression. By disrupting the anchor sequence-mediated conjunction, expression of the CXCL9, CXCL10, and/or CXCL11 gene may be decreased, e.g., decreased transcription due to conformational changes of the DNA previously open to transcription within the anchor sequence-mediated conjunction, e.g., decreased transcription due to conformational changes of the DNA creating additional distance between the CXCL9, CXCL10, and/or CXCL11 gene and the enhancing sequences. In one embodiment, both the CXCL9, CXCL10, and/or CXCL11 gene associated and one or more transcriptional control sequences, e.g., enhancing sequences, reside inside the anchor sequence-mediated conjunction. Disruption of the anchor sequence-mediated conjunction decreases expression of the CXCL9, CXCL10, and/or CXCL11 gene. In one embodiment, the CXCL9, CXCL10, and/or CXCL11 gene associated with the anchor sequence-mediated conjunction is accessible to one or more transcriptional control elements that reside inside, at least partially, the anchor sequence-mediated conjunction.

In some embodiments, expression of the CXCL9, CXCL10, and/or CXCL11 gene is regulated, modulated, or influenced by one or more transcriptional control elements associated with, but inaccessible due to the anchor sequence-mediated conjunction. For example, the anchor sequence-mediated conjunction associated with a CXCL9, CXCL10, and/or CXCL11 gene disrupts the ability of one or more transcriptional control elements to regulate, modulate, or influence expression of the CXCL9, CXCL10, and/or CXCL11 gene. The transcriptional control sequences may be separated from the CXCL9, CXCL10, and/or CXCL11 gene, e.g., reside on the opposite side, at least partially, e.g., inside or outside, of the anchor sequence-mediated conjunction as the CXCL9, CXCL10, and/or CXCL11 gene, e.g., the CXCL9, CXCL10, and/or CXCL11 gene is inaccessible to the transcriptional control elements due to proximity of the anchor sequence-mediated conjunction. In some embodiments, one or more enhancing sequences are separated from the CXCL9, CXCL10, and/or CXCL11 gene by the anchor sequence-mediated conjunction, e.g., a Type 2 anchor sequence-mediated conjunction.

In some embodiments, the CXCL9, CXCL10, and/or CXCL11 gene is inaccessible to one or more transcriptional control elements due to the anchor sequence-mediated conjunction, and disruption of the anchor sequence-mediated conjunction allows the transcriptional control element to regulate, modulate, or influence expression of the CXCL9, CXCL10, and/or CXCL11 gene. In one embodiment, the CXCL9, CXCL10, and/or CXCL11 gene is inside the anchor sequence-mediated conjunction and inaccessible to the one or more transcriptional control elements residing outside, at least partially, the anchor sequence-mediated conjunction. Disruption of the anchor sequence-mediated conjunction decreases expression of the CXCL9, CXCL10, and/or CXCL11 gene.

### Site-Specific Targeting Moieties

The site-specific targeting moieties of the invention target an expression control region associated with CXCL9, CXCL10, and/or CXCL11 and may comprise a polymer or polymeric molecule, such as a polyamide (i.e., a molecule of repeating units linked by amide binds, e.g., a polypeptide), a polymer of nucleotides (such as a guide RNA, a nucleic acid molecule encoding a TALE polypeptide or a zinc finger polypeptides), a peptide nucleic acid (PNA), or a polymer of amino acids, such as a peptide or polypeptide, e.g., a fusion protein, etc. Suitable site-specific targeting moieties, compositions, and methods of use of such agents and compositions are described below and in PCT Publication WO 2018/049073, the entire contents of which are expressly incorporated herein by reference.

In another embodiment, a site-specific epigenetic modifying agent of the invention comprises a site-specific targeting moiety comprising a nucleic acid molecule, such as a guide RNA (or gRNA) or a guide RNA and an effector, or fragment thereof, or nucleic acid molecule encoding an effector, or fragment thereof.

In one embodiment, a site specific epigenetic modifying agent of the invention comprises a site-specific targeting moiety comprising a nucleic acid molecule encoding a polypeptide, such as a DNA-binding domain, of a Transcription activator-like effector (TALE) polypeptide or a zinc finger (ZFP) polypeptide, or fragment thereof, that is engineered to specifically target an expression control region associated with CXCL9, CXCL10, and/or CXCL11 to reduce expression of a CXCL9, CXCL10, and/or CXCL11 gene.

In another embodiment, a site-specific epigenetic modifying agent of the invention comprises a site-specific targeting moiety comprising a polynucleotide, such as a PNA, e.g., a nucleic acid gRNA linked to an effector polypeptide, or fragment thereof.

In another embodiment, a site-specific epigenetic modifying agent of the invention comprises a site-specific targeting moiety comprising a fusion molecule, such as a nucleic acid molecule encoding a DNA-binding domain, of a Transcription activator-like effector (TALE) polypeptide or a zinc finger (ZFP) polypeptide, or fragment thereof, and an effector.

In one embodiment, such site-specific epigenetic modifying agents comprise a second fusion protein, wherein the second fusion protein comprises a second site-specific targeting moiety which targets a second expression control region associated with CXCL9, CXCL10, and/or CXCL11 and a second effector molecule, wherein the second expression control region associated with CXCL9, CXCL10, and/or CXCL11 is different than the expression control region associated with CXCL9, CXCL10, and/or CXCL11.

In another embodiment, a site-specific epigenetic modifying agent of the invention comprises a site-specific targeting moiety comprising a fusion molecule, such as a nucleic acid molecule encoding a fusion protein comprising a Cas polypeptide and, e.g., an epigenetic recruiter or an epigenetic CpG modifier.

In yet, another embodiment, a site-specific epigenetic modifying agent of the invention comprises a site-specific targeting moiety comprising a fusion molecule, such as fusion protein comprising a Cas polypeptide and, e.g., an epigenetic recruiter or an epigenetic CpG modifier.

As used herein, in its broadest sense, the term “nucleic acid” refers to any compound and/or substance that is or can be incorporated into an oligonucleotide chain. In some embodiments, a nucleic acid is a compound and/or substance that is or can be incorporated into a polynucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to a polynucleotide chain comprising individual nucleic acid residues. In some embodiments, a “nucleic acid” is or comprises RNA; in some embodiments, a “nucleic acid” is or comprises DNA. In some embodiments, a “nucleic acid” is a “mixmer” comprising locked nucleic acid molecules and deoxynucleic acid molecules. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof) (Examples in Table 1). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

TABLE 1
Abbreviations of nucleotide monomers used in nucleic
acid sequence representation. It will be understood
that these monomers, when present in an oligonucleotide,
are mutually linked by 5′-3′-phosphodiester bonds.

| Abbreviation | Nucleotide(s) |
|---|---|
| A | Adenosine-3′-phosphate |
| As | adenosine-3′-phosphorothioate |
| C | cytidine-3′-phosphate |
| Cs | cytidine-3′-phosphorothioate |
| G | guanosine-3′-phosphate |
| Gs | guanosine-3′-phosphorothioate |
| U | Uridine-3′-phosphate |
| Us | uridine-3′-phosphorothioate |
| N | any nucleotide, modified or unmodified |
| mA | 2′-O-methyladenosine-3′-phosphate |
| mAs | 2′-O-methyladenosine-3′-phosphorothioate |
| mC | 2′-O-methylcytidine-3′-phosphate |
| mCs | 2′-O-methylcytidine-3′-phosphorothioate |
| mG | 2′-O-methylguanosine-3′-phosphate |
| mGs | 2′-O-methylguanosine-3′-phosphorothioate |
| mU | 2′-O-methyluridine-3′-phosphate |
| mUs | 2′-O-methyluridine-3′-phosphorothioate |
| s | phosphorothioate linkage |

As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types.

In certain embodiments, a polypeptide is or may comprise a chimeric or “fusion protein.” As used herein, a “chimeric protein” or “fusion protein” comprises all or part (preferably a biologically active part) of a first protein operably linked to a heterologous second polypeptide (i.e., a polypeptide other than the first protein). Within the fusion protein, the term “operably linked” is intended to indicate that the first protein or segment thereof and the heterologous polypeptide are fused in-frame to each other. The heterologous polypeptide can be fused to the amino-terminus or the carboxyl-terminus of the first protein or segment.

A “polyamide” is a polymeric molecule with repeating units linked by amide binds. Proteins are examples of naturally occurring polyamides. In some embodiments, a polyamide comprises a peptide nucleic acid (PNA).

A “peptide nucleic acid” (“PNA”) is a molecule in which one or more amino acid units in the PNA have an amide containing backbone, e.g., aminoethyl-glycine, similar to a peptide backbone, with a nucleic acid side chain in place of the amino acid side chain. Peptide nucleic acids (PNA) are known to hybridize complementary DNA and RNA with higher affinity than their oligonucleotide counterparts. This character of PNA not only makes them a stable hybrid with the nucleic acid side chains, but at the same time, the neutral backbone and hydrophobic side chains result in a hydrophobic unit within the polypeptide. The nucleic acid side chain includes, but is not limited to, a purine or a pyrimidine side chain such as adenine, cytosine, guanine, thymine and uracil. In one embodiment, the nucleic acid side chain includes a nucleoside analog as described herein.

In one embodiment, a site-specific targeting moiety of the invention comprises a polyamide. Suitable polyamides for use in the agents and compositions of the invention are known in the art.

In one embodiment, a site-specific targeting moiety of the invention comprises a polynucleotide. In some embodiments, the nucleotide sequence of the polynucleotide encodes an CXCL9, CXCL10, and/or CXCL11 gene or a CXCL9, CXCL10, and/or CXCL11 expression product. In some embodiments, the nucleotide sequence of the polynucleotide does not include a CXCL9, CXCL10, and/or CXCL11 coding sequence or a CXCL9, CXCL10, and/or CXCL11 expression product. For example, in some embodiments, a site-specific targeting moiety of the invention comprises a polynucleotide that hybridizes to a target expression control region, e.g., a promoter, an anchor sequence, or a DNA region around or proximally upstream of the transcription starting site. In some embodiments, the nucleotide sequence of the polynucleotide is a complement of a target DNA region around or proximally upstream of the transcription starting site, or has a sequence that is at least 80%, at least 85%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a complement of the target sequence.

The polynucleotides of the invention may include deoxynucleotides, ribonucleotides, modified deoxynucleotides, modified ribonucleotides (e.g., chemical modifications, such as modifications that alter the backbone linkages, sugar molecules, and/or nucleic acid bases), and artificial nucleic acids. In some embodiments, the polynucleotide includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mPvNA, rPvNA, modified RNA, miRNA, gRNA, and siRNA or other RNA or DNA molecules.

In some embodiments, the polynucleotides of the invention have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range there between.

The polynucleotides of the invention may include nucleosides, e.g., purines or pyrimidines, e.g., adenine, cytosine, guanine, thymine and uracil. In some embodiments, the polynucleotides include one or more nucleoside analogs. The nucleoside analog includes, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-]pyridine, and any others that can base pair with a purine or a pyrimidine side chain.

In some embodiments, the site-specific targeting moieties of the invention comprise a polynucleotide encoding a polypeptide that comprises a DNA-binding domain (DBD), or fragment thereof, of a zinc finger polypeptide (ZFP) or a transcription activator-like effector (TALE) polypeptide, that is engineered to specifically target an expression control region associated with CXCL9, CXCL10, and/or CXCL11 to reduce expression of a CXCL9, CXCL10, and/or CXCL11 gene.

The design and preparation of such zinc finger polypeptides which specifically bind to a DNA target region of interest, such as an expression control region associated with CXCL9, CXCL10, and/or CXCL11, is well known in the art. For example, zinc finger (ZFP) proteins contain a DNA binding motif that specifically binds a triplet of nucleotides. Thus, to design and prepare the site-specific targeting moieties of the invention, a modular assembly process which includes combining separate zinc finger DNA binding domains that can each recognize a specific 3-basepair DNA sequence to generate 3-finger, 4-, 5-, 6-, 7-, or 8-zinc finger polypeptide that recognizes specific target sites ranging from 9 basepairs to 24 basepairs in length may be used. Another suitable method may include 2-finger modules to generate ZFP polynucleotides with up to six individual zinc fingers. See, e.g., Shukla V K, et al., Nature. 459 (7245) 2009:437-41; Dreier B, et al., JBC. 280 (42) 2005:35588-97; Dreier B, et al, JBC 276 (31) 2001:29466-78; Bae K H, et al., Nature Biotechnology. 21 (3) 2003:275-80.

In some embodiments, a site-specific targeting moiety of the invention comprises a polynucleotide encoding a polypeptide that comprises a DNA-binding domain (DBD), or fragment thereof, of a zinc finger, that is engineered to specifically target an expression control region associated with CXCL9, CXCL10, and/or CXCL11 to reduce expression of a CXCL9, CXCL10, and/or CXCL11 gene. Exemplary amino acid sequences encoding a zinc finger that binds to a nucleotide triplet suitable for use in the present invention are provide in Table 2 below. (See, e.g., Gersbach et al., Synthetic Zinc Finger Proteins: The Advent of Targeted Gene Regulation and Genome Modification Technologies).

TABLE 2

| Amino Acid Sequence of Zing Finger | Nucleotide |
|---|---|
| DNA Binding Domain (Finger) | Triplet |
| RKDALRG (SEQ IS NO: 1) | TTG |
| TTGALTE (SEQ IS NO: 2) | CTT |
| QRHHLVE (SEQ IS NO: 3) | CTC |
| QNSTLTE (SEQ IS NO: 4) | CTA |
| RNDALTE (SEQ IS NO: 5) | CTG |
| HKNALQN (SEQ IS NO: 6) | ATT |
| RRSACRR (SEQ IS NO: 7) | ATC |
| QKSSLIA (SEQ IS NO: 8) | ATA |
| RRDELNV (SEQ IS NO: 9) | ATG |
| TSGSLVR (SEQ IS NO: 10) | GTT |
| DPGALVR (SEQ IS NO: 11) | GTC |
| QSSSLVR (SEQ IS NO: 12) | GTA |
| RSDELVR (SEQ IS NO: 13) | GTG |
| RLRDIQF (SEQ IS NO: 14) | TCT |
| RSDERKR (SEQ IS NO: 15) | TCC |
| RSDHLTT (SEQ IS NO: 16) | TCA |
| RLRALDR (SEQ IS NO: 17) | TCG |
| TKNSLTE (SEQ IS NO: 18) | CCT |
| SKKHLAE (SEQ IS NO: 19) | CCC |
| TSHSLTE (SEQ IS NO: 20) | CCA |
| RNDTLTE (SEQ IS NO: 21) | CCG |
| THLDLIR (SEQ IS NO: 22) | ACT |
| DKKDLTR (SEQ IS NO: 23) | ACC |
| SPADLTR (SEQ IS NO: 24) | ACA |
| RTDTLRD (SEQ IS NO: 25) | ACG |
| TSGELVR (SEQ IS NO: 26) | GCT |
| DCRDLAR (SEQ IS NO: 27) | GCC |
| QSGDLRR (SEQ IS NO: 28) | GCA |
| RSDDLVR (SEQ IS NO: 29) | GCG |
| ARGNLRT (SEQ IS NO: 30) | TAT |
| SRGNLKS (SEQ IS NO: 31) | TAC |
| QASNLIS (SEQ IS NO: 32) | TAA |
| REDNLHT (SEQ IS NO: 33) | TAG |
| TSGNLTE (SEQ IS NO: 34) | CAT |
| SKKALTE (SEQ IS NO: 35) | CAC |
| QSGNLTE (SEQ IS NO: 36) | CAA |
| RADNLTE (SEQ IS NO: 37) | CAG |
| TTGNLTV (SEQ IS NO: 38) | AAT |
| DSGNLRV (SEQ IS NO: 39) | AAC |
| QRANLRA (SEQ IS NO: 40) | AAA |
| RKDNLKN (SEQ IS NO: 41) | AAG |
| TSGNLVR (SEQ IS NO: 42) | GAT |
| DPGNLVR (SEQ IS NO: 43) | GAC |
| QSSNLVR (SEQ IS NO: 44) | GAA |
| RSDNLVR (SEQ IS NO: 45) | GAG |
| APKALGW (SEQ IS NO: 46) | TGC |
| QAGHLAS (SEQ IS NO: 47) | TGA |
| RSDHLTT (SEQ IS NO: 48) | TGG |
| SRRTCRA (SEQ IS NO: 49) | CGT |
| HTGHLLE (SEQ IS NO: 50) | CGC |
| QSGHLTE (SEQ IS NO: 51) | CGA |
| RSDKLTE (SEQ IS NO: 52) | CGG |
| HRTTLTN (SEQ IS NO: 53) | AGT |
| ERSHLRE (SEQ IS NO: 54) | AGC |
| QLAHLRA (SEQ IS NO: 55) | AGA |
| RSDHLTN (SEQ IS NO: 56) | AGG |
| TSGHLVR (SEQ IS NO: 57) | GGT |
| DPGHLVR (SEQ IS NO: 58) | GGC |
| QRAHLER (SEQ IS NO: 59) | GGA |
| RSDKLVR (SEQ IS NO: 60) | GGG |

A zinc finger DNA binding domain comprises an N-terminal region and a C-terminal region with the “fingers” that bind to the target DNA sequence in between. The N-terminal region generally is 7 amino acids in length. The C-terminal region is generally 6 amino acids in length. Thus, the N-terminal region generally comprises the amino acid sequence of X₁X₂X₃X₄X₅X₆X₇. “X” can be any amino acid. In some embodiments, the N-terminal region comprises the exemplary amino acid sequence of LEPGEKP (SEQ ID NO: 309). “X” can be any amino acid. The C-terminal region generally comprises the amino acid sequence of X₂₅X₂₆X₂₇X₂₈X₂₉X₃₀. In certain embodiments, the C-terminal region comprises the exemplary amino acid sequence of TGKKTS (SEQ ID NO: 310)

Each finger in the DNA binding domain is flanked by a N-terminal backbone located to the N-terminus of the finger and a C-terminal backbone located to the C-terminus of the finger. The N-terminal backbone of the finger generally is 11 amino acids long with two conservative cysteines (C) locate at 3^{rd} and 6^{th} positions. Thus, the N-terminal backbone of the finger generally comprises the amino acid sequence of X₈X₉CX₁₀X₁₁CX₁₂X₁₃X₁₄X₁₅X₁₆. “X” can be any amino acid. The C-terminal backbone of the finger generally is 5 amino acids long with two conservative histines (H) located at 1^{st} and 5^{th} positions. Thus, the C-terminal backbone of the finger generally comprises the amino acid sequence of HX₁₇X₁₈X₁₉H. “X” can be any amino acid. In some embodiments, the N-terminal backbone comprises the exemplary amino acid sequence of YKCPECGKSFS (SEQ ID NO: 61) and the C-terminal backbone comprises the exemplary amino acid sequence of HQRTH (SEQ ID NO: 62). Two “fingers” are linked through a linker. A linker generally is 5 amino acids in length and comprises the amino acid sequence of X₂₀X₂₁X₂₂X₂₃X₂₄. “X” can be any amino acid. In certain embodiments, the linker comprises the exemplary amino acid sequence of TGEKP (SEQ ID NO: 63). Thus, the zinc finger of a site specific epigenetic modifying agent has a structure as follows: (N-terminal backbone-finger-C-terminal backbone-linker) n and the zinc finger DNA binding domain of a site specific epigenetic modifying agent has a structure as follows: [N-terminal region (N-terminal backbone-finger-C-terminal backbone-linker) n-C-terminal region]. “N” represents the number of triplets of nucleotides to which the zinc finger DNA binding domain and, thus, to which the site specific epigenetic modifying agent binds.

The “finger” amino acid sequences of four nucleotide triplets are unknown, however, if such a triplet is identified in a target area of interest, two “linker span sequences”—linker span 1 and linker span 2—are useful to circumvent the issue. Linker span 1 is used to skip one base pair if a “finger” amino acid sequence of a triplet is not available. Linker span 2 is used to skip 2 base pairs if a “finger” amino acid sequence of a triplet is not available. Linker span 1 is generally 12 amino acids long. Linker span 2 is generally 16 amino acids long. Thus, linker span 1 generally comprises the amino acid sequence of X₃₁X₃₂X₃₃X₃₄X₃₅X₃₆X₃₇X₃₈X₃₉X₄₀X₄₁X₄₂. Linker span 2 generally comprises the amino acid sequence of X₄₃X₄₄X₄₅X₄₆X₄₇X₄₈X₄₉X₅₀X₅₁X₅₂X₅₃X₅₄X₅₅X₅₆X₅₇X₅₈. In some embodiments, linker span 1 comprises the amino acid sequence of THPRAPIPKPFQ (SEQ ID NO: 311). In certain embodiments, linker span 2 comprises the amino acid sequence of TPNPHRRTDPSHKPFQ (SEQ ID NO: 312). When linker span 1 and/or linker span 2 is used, the finger-linker span 1/span 2-finger comprises the structure as follows: N-terminal back bone-finger-C-terminal backbone-linker span 1/span 2-N-terminal backbone-finger-C-terminal backbone-linker.

Table 3 provides the amino acid sequence of exemplary zinc finger DNA binding domains of the epigenetic modifying agents comprising a zinc finger DNA binding domain described in the working examples below.

In some embodiments, a zinc finger DNA binding domain suitable for use in the epigenetic modifying agents of the invention comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid identity to the entire amino acid sequence of any one of the zinc finger DNA binding domains provided in Table 3.

TABLE 3

| Exemplary Zinc | | |
|---|---|---|
| Finger DBD | Amino Acid Sequence | Nucleotide sequence |
| DBD386 | LEPGEKPYKCPECGKSFSSK | CTGGAACCCGGAGAGAAGCCCTATAAGT |
| ZF24 | KHLAEHQRTHTGEKPYKCP | GTCCGGAATGCGGAAAGAGTTTCAGCTC |
| | ECGKSFSQNSTLTEHQRTHT | GAAGAAGCACCTCGCCGAGCACCAACG |
| | GEKPYKCPECGKSFSQSGDL | GACCCACACGGGAGAAAAGCCCTACAA |
| | RRHQRTHTGEKPYKCPECG | GTGCCCAGAGTGCGGAAAGTCGTTCAGC |
| | KSFSTHLDLIRHQRTHTGEK | CAAAACTCCACCCTGACCGAACACCAGC |
| | PYKCPECGKSFSDPGNLVRH | GGACTCATACCGGGGAGAAACCTTACAA |
| | QRTHTGEKPYKCPECGKSFS | GTGTCCAGAATGCGGGAAGTCGTTCTCC |
| | TSGNLTEHQRTHTGEKPYKC | CAATCCGGAGACTTGCGCAGACACCAGC |
| | PECGKSFSERSHLREHQRTH | GCACTCACACCGGCGAAAAACCGTACAA |
| | TGKKTS | ATGCCCGGAGTGTGGAAAGTCATTTTCC |
| | (SEQ ID NO: 400) | ACCCACCTGGATCTGATCAGGCATCAGC |
| | | GCACCCACACTGGCGAAAAGCCTTACAA |
| | | ATGTCCGGAGTGCGGCAAATCCTTCTCT |
| | | GACCCCGGCAACCTCGTGCGCCATCAGA |
| | | GGACTCACACTGGGGAGAAGCCTTATAA |
| | | GTGCCCCGAATGTGGAAAGAGCTTCTCC |
| | | ACCTCCGGCAACCTTACCGAGCATCAGC |
| | | GGACACACACCGGGGAAAAGCCGTACA |
| | | AGTGCCCTGAGTGTGGCAAATCATTCTC |
| | | CGAACGGAGCCATCTGCGGGAGCACCA |
| | | GAGAACCCACACCGGAAAGAAAACTTC |
| | | C (SEQ ID NO: 401) |
| DBD374 | LEPGEKPYKCPECGKSFSQR | CTGGAGCCGGGAGAGAAGCCTTATAAGT |
| ZF16 | AHLERHQRTHTGEKPYKCP | GTCCTGAGTGCGGCAAATCATTCTCCCA |
| | ECGKSFSRSDHLTNHQRTHT | ACGCGCCCACCTCGAGAGACACCAGAG |
| | GEKPYKCPECGKSFSRNDAL | AACCCACACCGGGGAAAAGCCGTACAA |
| | TEHQRTHTGEKPYKCPECG | GTGCCCTGAGTGTGGGAAGTCCTTTTCC |
| | KSFSRADNLTEHQRTHTGEK | CGGTCCGACCATTTGACCAACCATCAGC |
| | PYKCPECGKSFSSKKALTEH | GGACCCACACTGGGGAGAAGCCATACA |
| | QRTHTGEKPYKCPECGKSFS | AATGCCCAGAATGCGGCAAAAGCTTCTC |
| | RNDALTEHQRTHTGEKPYK | CCGGAACGACGCACTTACCGAACATCAG |
| | CPECGKSFSHKNALQNHQR | CGCACGCACACCGGAGAGAAACCCTATA |
| | THTGKKTS | AGTGCCCCGAATGTGGAAAGTCATTCTC |
| | (SEQ ID NO: 402) | CCGGGCCGACAACCTGACAGAGCACCA |
| | | GCGGACTCACACTGGAGAAAAGCCTTAC |
| | | AAGTGTCCCGAATGCGGGAAGTCGTTCA |
| | | GCTCGAAGAAGGCTCTGACCGAACACCA |
| | | GAGGACCCATACCGGCGAAAAGCCCTAT |
| | | AAATGCCCGGAGTGCGGAAAGAGCTTCA |
| | | GCAGGAACGATGCCCTCACCGAGCACCA |
| | | ACGCACTCATACTGGCGAAAAACCGTAC |
| | | AAATGTCCGGAATGCGGAAAGTCCTTCT |
| | | CGCACAAGAACGCGCTGCAGAATCACCA |
| | | GCGCACTCACACCGGCAAAAAGACCTCT |
| | | (SEQ ID NO: 403) |
| DBD360 | LEPGEKPYKCPECGKSFSDK | CTCGAGCCTGGCGAAAAGCCTTATAAGT |
| ZF07 | KDLTRHQRTHTGEKPYKCP | GCCCAGAATGCGGAAAGAGCTTCTCCGA |
| | ECGKSFSQSSSLVRHQRTHT | CAAGAAGGATCTGACCCGCCACCAAAG |
| | GEKPYKCPECGKSFSTTGNL | GACCCACACTGGGGAGAAGCCTTACAAG |
| | TVHQRTHTGEKPYKCPECG | TGTCCGGAGTGCGGAAAGTCCTTTTCGC |
| | KSFSTSGELVRHQRTHTGEK | AATCGTCCAGCCTGGTCCGGCACCAGAG |
| | PYKCPECGKSFSTTGNLTVH | GACGCACACCGGAGAGAAGCCATACAA |
| | QRTHTGEKPYKCPECGKSFS | ATGCCCGGAGTGTGGGAAGTCGTTCTCC |
| | TSHSLTEHQRTHTGEKPYKC | ACCACTGGAAACCTCACTGTGCACCAGC |
| | PECGKSFSTSGNLVRHQRTH | GCACGCATACTGGGGAAAAGCCCTATAA |
| | TGKKTS | GTGTCCAGAGTGCGGGAAGTCCTTCTCA |
| | (SEQ ID NO: 404) | ACCTCCGGCGAATTGGTCAGACACCAGC |
| | | GGACACACACCGGCGAGAAGCCCTACA |
| | | AGTGCCCTGAGTGCGGCAAAAGCTTCAG |
| | | CACTACCGGCAACCTGACTGTGCATCAG |
| | | CGGACCCATACCGGAGAAAAACCCTACA |
| | | AATGTCCCGAATGCGGCAAATCCTTCTC |
| | | CACCTCCCACTCGCTGACCGAACACCAG |
| | | AGAACCCATACTGGAGAAAAGCCGTAC |
| | | AAGTGCCCGGAATGTGGAAAGTCTTTCA |
| | | GCACCTCCGGAAACCTTGTGCGGCATCA |
| | | GCGCACTCACACCGGCAAAAAGACCTCA |
| | | (SEQ ID NO: 405) |
| DBD358 | LEPGEKPYKCPECGKSFSQS | CTGGAGCCCGGAGAGAAACCGTATAAGT |
| ZF05 | GHLTEHQRTHTGEKPYKCPE | GCCCGGAATGTGGAAAGTCCTTCTCCCA |
| | CGKSFSQRANLRAHQRTHT | ATCCGGCCACCTGACTGAGCATCAGCGC |
| | GEKPYKCPECGKSFSTKNSL | ACCCACACTGGCGAAAAGCCGTACAAGT |
| | TEHQRTHTGEKPYKCPECG | GCCCTGAGTGCGGGAAGTCGTTTAGCCA |
| | KSFSTSGSLVRHQRTHTGEK | GCGGGCGAACTTGCGCGCTCATCAGCGG |
| | PYKCPECGKSFSQSGNLTEH | ACTCATACCGGGGAGAAGCCCTATAAAT |
| | QRTHTGEKPYKCPECGKSFS | GCCCGGAGTGCGGAAAGAGCTTCAGCAC |
| | QAGHLASHQRTHTGEKPYK | CAAGAACTCCCTGACTGAACACCAGCGG |
| | CPECGKSFSQSGDLRRHQRT | ACCCACACCGGAGAGAAGCCTTACAAGT |
| | HTGKKTS | GTCCTGAATGCGGGAAATCCTTCTCCAC |
| | (SEQ ID NO: 406) | CTCCGGTTCACTCGTGCGCCACCAGAGA |
| | | ACGCACACCGGTGAAAAGCCTTATAAGT |
| | | GTCCAGAGTGCGGCAAAAGCTTCAGCCA |
| | | GTCCGGCAACCTCACCGAGCACCAACGG |
| | | ACTCACACTGGAGAAAAGCCCTACAAAT |
| | | GTCCGGAATGCGGCAAATCATTCTCGCA |
| | | AGCCGGTCACCTTGCCTCGCACCAGCGC |
| | | ACACACACCGGGGAAAAGCCATACAAA |
| | | TGCCCCGAATGCGGAAAGTCGTTCTCTC |
| | | AATCCGGCGACCTGAGAAGGCATCAGA |
| | | GGACCCATACCGGAAAGAAAACCTCC |
| | | (SEQ ID NO: 407) |

Similarly, the design and preparation of such TALE polypeptides which specifically bind to a DNA target region of interest, such as an expression control region associated with CXCL9, CXCL10, and/or CXCL11, is well known in the art. For example, the TALE DNA binding domain contains a repeated highly conserved 33-34 amino acid sequence with divergent 12th and 13th amino acids. These two positions, referred to as the Repeat Variable Diresidue (RVD), are highly variable and show a strong correlation with specific nucleotide recognition. This straightforward relationship between amino acid sequence and DNA recognition has allowed for the engineering of specific DNA-binding domains by selecting a combination of repeat segments containing the appropriate RVDs. See, e.g., Boch J Nature Biotechnology. 29 (2) 2011:135-6; Boch J, et al., Science. 326 (5959) 2009:1509-12; Moscou M J & Bogdanove A J Science. 326 (5959) 2009:1501.

In some embodiments, a TALE binding domain suitable for use in the epigenetic modifying agents of the invention comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid identity to the entire amino acid sequence of any one of the TALE binding domains provided in Table 4 (mouse) and Table 5 (human).

TABLE 4

| Exemplary TALE | | |
|---|---|---|
| DBD mouse | Amino Acid Sequence | Nucleotide sequence |
| DBD455 | HMVDLRTLGYSQQQQEKIKPK | CACATGGTGGACCTGAGAACTCTGGGT |
| TAL37 | VRSTVAQHHEALVGHGFTHA | TACTCCCAGCAACAGCAAGAAAAAAT |
| mouse | HIVALSQHPAALGTVAVKYQD | CAAGCCAAAAGTGCGCAGCACTGTCG |
| | MIAALPEATHEAIVGVGKRGA | CGCAGCACCACGAGGCCCTTGTGGGGC |
| | GARALEALLTVAGELRGPPLQ | ACGGGTTCACCCACGCTCACATCGTGG |
| | LDTGQLLKIAKRGGVTAVEAV | CCCTGAGCCAGCATCCGGCAGCCCTGG |
| | HAWRNALTGAPLNLTPEQVV | GAACCGTCGCAGTGAAGTACCAAGAC |
| | AIASNNGGKQALETVQRLLPV | ATGATCGCCGCCTTGCCGGAAGCCACT |
| | LCQAHGLTPEQVVAIASNIGG | CACGAAGCAATTGTGGGAGTGGGGAA |
| | KQALETVQRLLPVLCQAHGLT | GAGAGGAGCCGGCGCAAGAGCCCTCG |
| | PEQVVAIASNGGGKQALETVQ | AAGCGCTTCTGACTGTGGCCGGAGAAT |
| | RLLPVLCQAHGLTPEQVVAIA | TGAGAGGCCCCCCACTGCAGCTGGATA |
| | SNIGGKQALETVQRLLPVLCQ | CCGGACAACTGCTGAAGATCGCCAAA |
| | AHGLTPEQVVAIASNGGGKQA | CGGGGTGGAGTGACCGCCGTGGAAGC |
| | LETVQRLLPVLCQAHGLTPEQ | GGTGCACGCGTGGAGAAACGCGCTGA |
| | VVAIASHDGGKQALETVQRLL | CTGGAGCCCCGCTCAACCTGACCCCTG |
| | PVLCQAHGLTPEQVVAIASNG | AACAGGTCGTGGCTATTGCCTCAAACA |
| | GGKQALETVQRLLPVLCQAH | ACGGCGGGAAGCAAGCGTTGGAGACT |
| | GLTPEQVVAIASNIGGKQALET | GTACAGCGCCTGCTGCCAGTGTTGTGT |
| | VQRLLPVLCQAHGLTPEQVVA | CAAGCCCACGGGCTGACTCCCGAGCA |
| | IASNIGGKQALETVQRLLPVLC | AGTGGTGGCCATAGCCAGCAATATCGG |
| | QAHGLTPEQVVAIASNNGGKQ | AGGTAAACAGGCTCTCGAGACTGTCCA |
| | ALETVQRLLPVLCQAHGLTPE | ACGCCTGCTCCCAGTGCTTTGTCAGGC |
| | QVVAIASNGGGKQALETVQRL | TCACGGCCTCACTCCTGAACAGGTCGT |
| | LPVLCQAHGLTPEQVVAIASH | AGCCATTGCCAGCAACGGTGGCGGCA |
| | DGGKQALETVQRLLPVLCQA | AACAAGCCTTAGAGACTGTGCAACGG |
| | HGLTPEQVVAIASNIGGKQAL | CTCTTGCCGGTGCTCTGTCAGGCCCAC |
| | ETVQRLLPVLCQAHGLTPEQV | GGACTGACCCCAGAACAAGTAGTCGCT |
| | VAIASNNGGKQALETVQRLLP | ATCGCGTCAAACATCGGCGGCAAGCA |
| | VLCQAHGLTPEQVVAIASRHG | GGCTTTGGAAACCGTCCAGCGGCTTCT |
| | GKQALETVQRLLPVLCQAHGL | GCCGGTGCTGTGCCAGGCGCATGGGCT |
| | TPEQVVAIASNGGGKQALETV | CACCCCCGAACAGGTGGTCGCAATCGC |
| | QRLLPVLCQAHGLTPEQVVAI | CTCGAACGGCGGTGGTAAACAAGCCCT |
| | ASNGGGKQALETVQRLLPVLC | CGAAACTGTCCAGCGCCTCTTGCCCGT |
| | QAHGLTPEQVVAIASHDGGRP | CCTGTGTCAGGCCCATGGCCTGACTCC |
| | ALESIVAQLSRPDPALAALTND | AGAACAGGTGGTGGCAATAGCGTCCC |
| | HLVALACLGGRPALDAVKKG | ATGACGGGGGAAAGCAAGCCCTTGAG |
| | LPHAPALIKRTNRRIPERTSHR | ACTGTGCAACGGTTGCTCCCCGTGTTA |
| | V | TGTCAGGCACATGGTCTGACCCCCGAA |
| | (SEQ ID NO: 408) | CAGGTGGTCGCAATTGCGTCGAACGGC |
| | | GGAGGAAAGCAAGCTCTGGAAACGGT |
| | | CCAACGGCTGCTCCCTGTGCTGTGCCA |
| | | GGCGCACGGACTTACCCCAGAACAAG |
| | | TGGTCGCCATTGCGTCCAACATCGGAG |
| | | GGAAGCAGGCGCTTGAAACCGTGCAG |
| | | AGGTTGCTGCCGGTGTTATGCCAAGCA |
| | | CATGGACTAACCCCGGAGCAAGTGGT |
| | | GGCCATTGCCTCGAATATTGGGGGGAA |
| | | ACAAGCGTTAGAAACTGTGCAGAGGC |
| | | TGCTGCCTGTGCTCTGCCAAGCCCATG |
| | | GTCTTACCCCGGAACAGGTTGTGGCGA |
| | | TTGCATCCAACAACGGTGGAAAACAG |
| | | GCCCTTGAAACGGTGCAGCGGCTCCTT |
| | | CCTGTCCTGTGCCAAGCCCATGGACTC |
| | | ACCCCTGAACAGGTCGTGGCTATTGCC |
| | | TCCAACGGAGGCGGCAAGCAGGCCCT |
| | | GGAGACAGTCCAGAGACTGCTGCCCGT |
| | | GCTGTGTCAAGCTCACGGTCTGACTCC |
| | | GGAACAGGTGGTTGCTATCGCTTCGCA |
| | | CGACGGCGGAAAACAAGCTCTTGAAA |
| | | CCGTGCAAAGGCTGCTCCCGGTGCTTT |
| | | GCCAAGCTCATGGCTTGACCCCAGAGC |
| | | AAGTCGTGGCAATCGCGTCCAATATCG |
| | | GTGGGAAGCAGGCTCTTGAGACAGTG |
| | | CAAAGACTCCTCCCCGTCCTCTGTCAA |
| | | GCGCACGGGCTCACTCCTGAACAGGTC |
| | | GTCGCTATTGCTTCGAACAACGGGGGC |
| | | AAACAGGCACTAGAGACTGTCCAAAG |
| | | ACTTTTACCTGTCCTCTGCCAGGCACA |
| | | CGGATTGACTCCCGAACAAGTCGTCGC |
| | | GATTGCCTCAAGACACGGGGGCAAGC |
| | | AAGCGCTGGAGACTGTTCAGCGACTGC |
| | | TTCCTGTGCTTTGCCAGGCCCACGGTTT |
| | | GACCCCTGAGCAAGTIGTGGCCATCGC |
| | | CTCTAACGGAGGTGGAAAGCAGGCCTT |
| | | GGAAACAGTGCAGAGACTGTTGCCCGT |
| | | CTTGTGCCAGGCTCATGGGCTTACGCC |
| | | TGAACAAGTCGTCGCCATCGCTTCGAA |
| | | CGGAGGAGGGAAACAGGCGCTGGAAA |
| | | CTGTGCAGCGACTCCTTCCGGTCCTTT |
| | | GTCAAGCACACGGCCTTACGCCGGAAC |
| | | AAGTGGTTGCCATCGCAAGCCACGACG |
| | | GAGGACGCCCTGCCCTCGAGTCCATTG |
| | | TGGCCCAGCTGTCACGCCCAGATCCTG |
| | | CACTGGCCGCTCTTACCAACGATCACC |
| | | TGGTCGCTCTCGCGTGCCTGGGAGGAA |
| | | GGCCTGCCCTGGATGCCGTCAAGAAGG |
| | | GACTGCCTCATGCCCCTGCTTTGATTA |
| | | AGAGGACTAATCGGCGCATCCCCGAA |
| | | CGGACCAGCCACAGGGTG |
| | | (SEQ ID NO: 409) |
| DBD452 | HMVDLRTLGYSQQQQEKIKPK | CACATGGTGGACCTGAGAACTCTGGGT |
| TAL34 | VRSTVAQHHEALVGHGFTHA | TACTCCCAGCAACAGCAAGAAAAAAT |
| mouse | HIVALSQHPAALGTVAVKYQD | CAAGCCAAAAGTGCGCAGCACTGTCG |
| | MIAALPEATHEAIVGVGKRGA | CGCAGCACCACGAGGCCCTTGTGGGGC |
| | GARALEALLTVAGELRGPPLQ | ACGGGTTCACCCACGCTCACATCGTGG |
| | LDTGQLLKIAKRGGVTAVEAV | CCCTGAGCCAGCATCCGGCAGCCCTGG |
| | HAWRNALTGAPLNLTPEQVV | GAACCGTCGCAGTGAAGTACCAAGAC |
| | AIASNIGGKQALETVQRLLPVL | ATGATCGCCGCCTTGCCGGAAGCCACT |
| | CQAHGLTPEQVVAIASNGGGK | CACGAAGCAATTGTGGGAGTGGGGAA |
| | QALETVQRLLPVLCQAHGLTP | GAGAGGAGCCGGCGCAAGAGCCCTCG |
| | EQVVAIASHDGGKQALETVQR | AAGCGCTTCTGACTGTGGCCGGAGAAT |
| | LLPVLCQAHGLTPEQVVAIAS | TGAGAGGCCCCCCACTGCAGCTGGATA |
| | HDGGKQALETVQRLLPVLCQ | CCGGACAACTGCTGAAGATCGCCAAA |
| | AHGLTPEQVVAIASHDGGKQA | CGGGGTGGAGTGACTGCCGTCGAGGCT |
| | LETVQRLLPVLCQAHGLTPEQ | GTGCACGCCTGGAGAAACGCCTTGACC |
| | VVAIASNGGGKQALETVQRLL | GGAGCGCCACTGAACCTTACCCCGGAG |
| | PVLCQAHGLTPEQVVAIASNG | CAAGTGGTGGCGATAGCATCAAATATT |
| | GGKQALETVQRLLPVLCQAH | GGCGGAAAGCAGGCCCTGGAAACCGT |
| | GLTPEQVVAIASNIGGKQALET | ACAAAGACTGTTGCCGGTCCTGTGTCA |
| | VQRLLPVLCQAHGLTPEQVVA | AGCTCATGGTTTGACCCCAGAACAGGT |
| | IASHDGGKQALETVQRLLPVL | CGTGGCTATTGCATCGAACGGCGGAGG |
| | CQAHGLTPEQVVAIASNGGGK | CAAACAAGCCCTCGAGACAGTCCAGA |
| | QALETVQRLLPVLCQAHGLTP | GACTTCTGCCGGTTCTGTGCCAGGCGC |
| | EQVVAIASNIGGKQALETVQR | ACGGACTTACCCCTGAACAGGTGGTGG |
| | LLPVLCQAHGLTPEQVVAIAS | CGATTGCGAGCCACGACGGTGGTAAA |
| | NGGGKQALETVQRLLPVLCQ | CAGGCGTTGGAAACTGTGCAGCGGTTG |
| | AHGLTPEQVVAIASNIGGKQA | CTTCCGGTGCTGTGTCAGGCTCACGGG |
| | LETVQRLLPVLCQAHGLTPEQ | CTGACCCCCGAACAAGTGGTAGCTATC |
| | VVAIASNIGGKQALETVQRLLP | GCCTCCCACGACGGAGGGAAGCAGGC |
| | VLCQAHGLTPEQVVAIASNIG | TCTGGAAACGGTGCAACGCCTCCTTCC |
| | GKQALETVQRLLPVLCQAHGL | CGTCTTGTGCCAAGCACATGGACTGAC |
| | TPEQVVAIASHDGGKQALETV | TCCAGAACAGGTCGTCGCCATTGCCTC |
| | QRLLPVLCQAHGLTPEQVVAI | CCATGATGGGGGGAAGCAGGCACTGG |
| | ASNGGGKQALETVQRLLPVLC | AAACTGTACAACGCCTGCTGCCTGTCC |
| | QAHGLTPEQVVAIASHDGGRP | TCTGCCAAGCGCACGGTTTGACGCCGG |
| | ALESIVAQLSRPDPALAALTND | AACAGGTTGTCGCCATCGCCTCGAACG |
| | HLVALACLGGRPALDAVKKG | GAGGGGGAAAACAGGCCCTTGAAACA |
| | LPHAPALIKRTNRRIPERTSHR | GTGCAACGGCTGCTGCCAGTGCTGTGT |
| | V | CAGGCGCATGGCTTGACCCCGGAGCA |
| | (SEQ ID NO: 410) | AGTGGTGGCCATAGCCTCGAATGGAG |
| | | GAGGAAAGCAGGCCCTGGAAACCGTT |
| | | CAGCGCCTCCTCCCTGTGCTGTGTCAG |
| | | GCCCATGGGCTCACTCCCGAACAGGTA |
| | | GTCGCCATAGCGTCGAACATCGGAGG |
| | | AAAACAAGCGCTCGAAACTGTCCAAA |
| | | GACTGCTTCCAGTCCTTTGCCAGGCTC |
| | | ATGGACTAACTCCAGAGCAAGTAGTG |
| | | GCCATCGCGAGCCATGACGGCGGCAA |
| | | GCAAGCACTCGAAACCGTGCAGAGGC |
| | | TGCTTCCCGTCCTGTGCCAAGCTCACG |
| | | GCCTGACGCCTGAACAGGTGGTGGCTA |
| | | TCGCCAGCAACGGTGGTGGAAAGCAG |
| | | GCGCTCGAGACTGTGCAGAGGTTGCTC |
| | | CCAGTGCTCTGTCAGGCACATGGCCTC |
| | | ACCCCGGAGCAAGTGGTGGCAATCGC |
| | | CTCAAACATCGGTGGCAAACAGGCACT |
| | | TGAAACCGTGCAGCGCCTTTTGCCTGT |
| | | GCTCTGCCAGGCCCACGGACTGACCCC |
| | | TGAGCAAGTCGTGGCTATTGCCTCTAA |
| | | CGGCGGTGGCAAGCAGGCCTTGGAAA |
| | | CCGTCCAGAGGCTCTTGCCGGTGCTCT |
| | | GCCAGGCACACGGACTCACTCCGGAA |
| | | CAGGTGGTCGCCATCGCATCGAATATC |
| | | GGCGGAAAACAGGCCCTCGAAACGGT |
| | | CCAGCGGTTACTCCCCGTGCTTTGTCA |
| | | GGCTCACGGATTGACTCCCGAGCAAGT |
| | | CGTGGCCATTGCTTCCAACATTGGGGG |
| | | GAAACAGGCGCTGGAAACGGTGCAGC |
| | | GACTCCTTCCTGTGTTGTGTCAAGCCC |
| | | ACGGCCTCACTCCAGAGCAAGTCGTGG |
| | | CAATTGCCTCCAACATCGGGGGAAAGC |
| | | AAGCCTTGGAGACAGTACAGCGGCTG |
| | | CTTCCTGTGCTTTGCCAAGCCCATGGA |
| | | CTCACCCCCGAACAAGTGGTTGCCATT |
| | | GCCAGCCACGATGGAGGGAAACAAGC |
| | | CCTGGAGACAGTGCAAAGGCTCCTGCC |
| | | CGTGCTGTGCCAGGCACACGGCTTGAC |
| | | TCCTGAACAAGTCGTCGCTATCGCTTC |
| | | CAATGGTGGTGGGAAGCAAGCTCTGG |
| | | AGACTGTCCAGCGGCTCCTGCCAGTGT |
| | | TGTGTCAAGCACACGGTCTGACCCCGG |
| | | AACAAGTCGTTGCAATCGCGTCGCATG |
| | | ATGGAGGACGCCCTGCCCTCGAGTCCA |
| | | TTGTGGCCCAGCTGTCACGCCCAGATC |
| | | CTGCACTGGCCGCTCTTACCAACGATC |
| | | ACCTGGTCGCTCTCGCGTGCCTGGGAG |
| | | GAAGGCCTGCCCTGGATGCCGTCAAGA |
| | | AGGGACTGCCTCATGCCCCTGCTTTGA |
| | | TTAAGAGGACTAATCGGCGCATCCCCG |
| | | AACGGACCAGCCACAGGGTG |
| | | (SEQ ID NO: 411) |
| DBD429 | HMVDLRTLGYSQQQQEKIKPK | CACATGGTGGACCTCCGCACGCTGGGA |
| TAL27 | VRSTVAQHHEALVGHGFTHA | TACTCGCAACAGCAGCAGGAGAAGAT |
| mouse | HIVALSQHPAALGTVAVKYQD | TAAGCCTAAGGTCCGGTCCACCGTGGC |
| | MIAALPEATHEAIVGVGKRGA | TCAGCACCATGAGGCGCTAGTGGGCCA |
| | GARALEALLTVAGELRGPPLQ | CGGATTCACCCACGCGCATATTGTCGC |
| | LDTGQLLKIAKRGGVTAVEAV | CCTCTCGCAACATCCCGCCGCACTGGG |
| | HAWRNALTGAPLNLTPEQVV | AACCGTCGCCGTGAAGTACCAGGACAT |
| | AIASNIGGKQALETVQRLLPVL | GATTGCCGCCCTGCCTGAAGCTACCCA |
| | CQAHGLTPEQVVAIASNIGGK | TGAAGCCATTGTGGGCGTGGGAAAGA |
| | QALETVQRLLPVLCQAHGLTP | GAGGAGCAGGGGCCAGAGCCTTGGAG |
| | EQVVAIASNNGGKQALETVQR | GCCCTGCTCACGGTAGCCGGAGAGCTC |
| | LLPVLCQAHGLTPEQVVAIAS | AGAGGCCCACCACTGCAGCTTGACACC |
| | NIGGKQALETVQRLLPVLCQA | GGCCAACTTCTGAAGATCGCCAAGCGG |
| | HGLTPEQVVAIASNGGGKQAL | GGAGGAGTGACCGCCGTGGAGGCAGT |
| | ETVQRLLPVLCQAHGLTPEQV | GCATGCCTGGCGCAACGCCTTGACTGG |
| | VAIASNIGGKQALETVQRLLPV | AGCCCCGCTGAACCTGACTCCGGAGCA |
| | LCQAHGLTPEQVVAIASNNGG | AGTGGTGGCTATCGCGTCCAACATCGG |
| | KQALETVQRLLPVLCQAHGLT | AGGAAAACAGGCCCTGGAAACGGTGC |
| | PEQVVAIASNIGGKQALETVQ | AAAGACTGCTGCCGGTCCTCTGTCAAG |
| | RLLPVLCQAHGLTPEQVVAIA | CGCACGGACTCACTCCCGAGCAAGTTG |
| | SNGGGKQALETVQRLLPVLCQ | TGGCTATCGCTTCCAACATCGGGGGGA |
| | AHGLTPEQVVAIASNNGGKQA | AACAGGCTCTGGAAACTGTCCAAAGG |
| | LETVQRLLPVLCQAHGLTPEQ | CTGTTACCTGTGCTGTGCCAAGCACAC |
| | VVAIASNIGGKQALETVQRLLP | GGACTGACCCCAGAGCAAGTGGTCGC |
| | VLCQAHGLTPEQVVAIASNIG | CATAGCCTCCAACAACGGGGGGAAGC |
| | GKQALETVQRLLPVLCQAHGL | AAGCCCTGGAGACTGTGCAGCGGTTGC |
| | TPEQVVAIASNNGGKQALETV | TCCCCGTCCTGTGCCAGGCCCACGGAT |
| | QRLLPVLCQAHGLTPEQVVAI | TGACTCCCGAACAAGTGGTGGCCATTG |
| | ASRHGGKQALETVQRLLPVLC | CGTCAAACATCGGGGGCAAGCAAGCG |
| | QAHGLTPEQVVAIASNGGGKQ | TTGGAAACAGTCCAACGGTTGCTTCCC |
| | ALETVQRLLPVLCQAHGLTPE | GTCCTTTGCCAGGCCCACGGTCTGACC |
| | QVVAIASHDGGKQALETVQRL | CCGGAGCAAGTCGTCGCCATTGCTAGT |
| | LPVLCQAHGLTPEQVVAIASNI | AACGGGGGAGGAAAGCAAGCTCTTGA |
| | GGKQALETVQRLLPVLCQAH | AACCGTCCAGCGGCTTCTTCCGGTGCT |
| | GLTPEQVVAIASHDGGRPALE | CTGCCAAGCACACGGCCTCACACCCGA |
| | SIVAQLSRPDPALAALTNDHL | GCAAGTAGTGGCCATTGCCTCAAACAT |
| | VALACLGGRPALDAVKKGLP | TGGGGGAAAGCAGGCATTGGAAACTG |
| | HAPALIKRTNRRIPERTSHRV | TGCAACGGCTGCTTCCTGTCTTGTGCC |
| | (SEQ ID NO: 412) | AGGCGCATGGGCTGACTCCAGAACAG |
| | | GTGGTCGCTATCGCGAGTAACAACGGT |
| | | GGCAAACAGGCACTGGAAACCGTGCA |
| | | GAGGCTCTTGCCTGTCCTGTGTCAGGC |
| | | ACACGGACTGACGCCAGAACAGGTGG |
| | | TCGCTATTGCGAGCAATATCGGCGGAA |
| | | AGCAGGCGTTAGAAACTGTGCAGAGA |
| | | CTGCTCCCAGTGCTATGCCAGGCTCAT |
| | | GGGTTGACTCCTGAACAAGTGGTGGCT |
| | | ATCGCCTCAAACGGTGGCGGTAAACA |
| | | GGCGCTTGAGACAGTGCAAAGGCTCCT |
| | | TCCCGTGTTGTGCCAAGCGCATGGCCT |
| | | GACCCCTGAACAGGTCGTGGCTATCGC |
| | | GTCAAACAACGGAGGCAAGCAGGCCC |
| | | TAGAAACGGTACAACGGCTGCTGCCCG |
| | | TACTGTGCCAGGCCCACGGACTGACCC |
| | | CAGAACAGGTGGTCGCAATTGCCTCCA |
| | | ACATTGGCGGGAAGCAGGCGCTAGAA |
| | | ACCGTGCAGCGGCTTCTGCCCGTCCTC |
| | | TGCCAGGCCCACGGTCTGACTCCTGAG |
| | | CAAGTCGTCGCAATCGCCAGCAACATC |
| | | GGCGGAAAACAAGCCCTCGAAACCGT |
| | | CCAGCGACTGCTGCCTGTCCTATGCCA |
| | | GGCCCACGGGTTGACCCCGGAACAGG |
| | | TCGTCGCCATTGCTTCCAATAATGGAG |
| | | GCAAACAGGCACTCGAAACGGTGCAG |
| | | AGACTTCTGCCGGTGCTGTGTCAAGCT |
| | | CACGGCTTGACCCCCGAGCAAGTGGTG |
| | | GCAATCGCTTCACGACACGGTGGAAA |
| | | ACAGGCCCTGGAGACAGTCCAGAGAC |
| | | TGCTCCCCGTGCTTTGCCAAGCTCACG |
| | | GACTTACTCCGGAACAAGTCGTGGCGA |
| | | TTGCGTCCAACGGTGGTGGAAAGCAG |
| | | GCCTTGGAAACGGTCCAGAGACTGTTG |
| | | CCGGTCTTGTGCCAGGCGCATGGTCTG |
| | | ACCCCCGAACAAGTCGTGGCAATTGCA |
| | | TCCCATGACGGCGGGAAACAGGCTTTG |
| | | GAGACTGTCCAGCGCCTGTTGCCAGTC |
| | | CTTTGTCAAGCCCACGGGTTGACACCC |
| | | GAACAGGTCGTCGCAATCGCCTCGAAC |
| | | ATCGGTGGCAAGCAGGCCCTTGAAACT |
| | | GTCCAACGCCTCCTCCCGGTGTTGTGT |
| | | CAGGCTCACGGCCTGACTCCAGAACAA |
| | | GTCGTGGCCATAGCGTCACACGACGGT |
| | | GGACGGCCAGCCCTGGAATCCATCGTG |
| | | GCGCAGCTGTCCAGACCCGACCCAGCA |
| | | CTGGCGGCCCTCACTAACGATCACCTT |
| | | GTGGCCCTGGCCTGTCTGGGAGGTAGA |
| | | CCAGCCCTCGACGCTGTGAAGAAGGG |
| | | ACTGCCGCATGCACCAGCCCTGATCAA |
| | | GCGAACCAACCGGCGCATTCCCGAGA |
| | | GAACTTCACACAGAGTG |
| | | (SEQ ID NO: 413) |

TABLE 5

| Exemplary | | |
|---|---|---|
| TALE DBD human | Amino Acid Sequence | Nucleotide Sequence |
| DBD330 | HMVDLRTLGYSQQQQEKIKP | CACATGGTCGATCTTCGCACCCTCGGG |
| TAL14 | KVRSTVAQHHEALVGHGFTH | TACTCGCAGCAGCAACAGGAAAAGAT |
| human | AHIVALSQHPAALGTVAVKY | CAAGCCGAAAGTCAGGTCCACCGTCGC |
| | QDMIAALPEATHEAIVGVGKR | CCAGCACCACGAGGCACTGGTCGGCC |
| | GAGARALEALLTVAGELRGPP | ACGGATTCACCCACGCCCACATCGTCG |
| | LQLDTGQLLKIAKRGGVTAVE | CGCTGTCTCAACATCCAGCCGCCCTGG |
| | AVHAWRNALTGAPLNLTPEQ | GAACTGTCGCCGTCAAGTACCAAGATA |
| | VVAIASNIGGKQALETVQRLL | TGATCGCGGCACTGCCTGAAGCAACCC |
| | PVLCQAHGLTPEQVVAIASHD | ATGAGGCCATCGTGGGTGTGGGAAAG |
| | GGKQALETVQRLLPVLCQAH | AGAGGAGCAGGGGCTAGAGCCCTGGA |
| | GLTPEQVVAIASHDGGKQALE | AGCGCTCCTGACCGTGGCGGGAGAGCT |
| | TVQRLLPVLCQAHGLTPEQVV | TAGAGGCCCTCCCCTGCAATTGGACAC |
| | AIASNGGGKQALETVQRLLPV | CGGCCAACTTCTGAAGATTGCAAAAAG |
| | LCQAHGLTPEQVVAIASNGGG | GGGTGGAGTGACTGCCGTGGAAGCAG |
| | KQALETVQRLLPVLCQAHGL | TGCACGCTTGGCGGAACGCACTGACTG |
| | TPEQVVAIASNIGGKQALETV | GAGCACCCCTGAATTTGACCCCCGAGC |
| | QRLLPVLCQAHGLTPEQVVAI | AAGTGGTTGCCATCGCAAGCAACATCG |
| | ASNIGGKQALETVQRLLPVLC | GAGGGAAACAAGCACTGGAAACTGTG |
| | QAHGLTPEQVVAIASNNGGK | CAGCGCCTGCTTCCGGTCCTGTGCCAA |
| | QALETVQRLLPVLCQAHGLTP | GCTCACGGACTGACTCCTGAACAGGTT |
| | EQVVAIASNIGGKQALETVQR | GTGGCCATCGCGTCGCATGACGGGGG |
| | LLPVLCQAHGLTPEQVVAIAS | AAAACAGGCGCTCGAAACCGTCCAAA |
| | NIGGKQALETVQRLLPVLCQA | GACTGCTCCCGGTGCTGTGTCAGGCTC |
| | HGLTPEQVVAIASNNGGKQA | ACGGTTTGACCCCAGAACAAGTIGTCG |
| | LETVQRLLPVLCQAHGLTPEQ | CTATCGCCTCACACGACGGCGGGAAGC |
| | VVAIASRHGGKQALETVQRLL | AAGCTCTGGAGACTGTGCAAAGACTCC |
| | PVLCQAHGLTPEQVVAIASHD | TTCCTGTGCTGTGCCAAGCTCATGGAT |
| | GGKQALETVQRLLPVLCQAH | TGACCCCGGAACAGGTCGTCGCCATCG |
| | GLTPEQVVAIASNGGGKQALE | CTTCCAATGGCGGAGGTAAACAGGCCT |
| | TVQRLLPVLCQAHGLTPEQVV | TGGAAACGGTGCAGAGGCTGCTGCCA |
| | AIASNNGGKQALETVQRLLPV | GTCTTGTGTCAGGCGCACGGGCTGACT |
| | LCQAHGLTPEQVVAIASRHGG | CCAGAGCAAGTAGTAGCCATCGCCAG |
| | KQALETVQRLLPVLCQAHGL | CAATGGAGGCGGTAAACAAGCGCTCG |
| | TPEQVVAIASNGGGKQALETV | AGACCGTCCAGCGCTTGTTGCCAGTGT |
| | QRLLPVLCQAHGLTPEQVVAI | TGTGCCAGGCCCATGGGCTGACGCCCG |
| | ASNGGGRPALESIVAQLSRPD | AACAGGTGGTGGCCATTGCATCCAACA |
| | PALAALTNDHLVALACLGGR | TTGGGGGGAAACAGGCGTTGGAGACA |
| | PALDAVKKGLPHAPALIKRTN | GTCCAACGGCTTCTGCCCGTCCTTTGTC |
| | RRIPERTSHRV | AGGCACACGGACTCACTCCTGAACAA |
| | (SEQ ID NO: 414) | GTCGTTGCCATTGCCTCGAATATCGGG |
| | | GGTAAGCAAGCCCTCGAGACTGTGCA |
| | | GAGATTGTTGCCTGTCCTGTGTCAAGC |
| | | GCATGGTCTGACTCCCGAACAGGTGGT |
| | | GGCGATTGCTTCGAACAATGGAGGAA |
| | | AACAAGCATTGGAAACCGTCCAGAGG |
| | | CTGTTACCAGTCCTTTGCCAGGCACAT |
| | | GGACTGACCCCGGAACAGGTCGTCGC |
| | | AATCGCATCGAACATTGGAGGCAAGC |
| | | AGGCTCTCGAAACGGTCCAGAGACTGC |
| | | TGCCTGTGCTCTGCCAAGCCCACGGCC |
| | | TCACTCCGGAGCAAGTCGTGGCAATCG |
| | | CCTCAAACATCGGTGGAAAGCAGGCC |
| | | CTTGAGACTGTCCAGCGACTGCTCCCA |
| | | GTGCTCTGCCAGGCGCATGGACTCACA |
| | | CCCGAGCAGGTGGTCGCCATTGCTAGC |
| | | AACAACGGAGGCAAACAAGCCCTGGA |
| | | GACAGTGCAACGCTTGCTGCCCGTGCT |
| | | TTGTCAAGCGCACGGACTTACTCCGGA |
| | | ACAAGTAGTGGCTATTGCGAGTAGGCA |
| | | CGGCGGTAAGCAAGCCCTGGAGACAG |
| | | TGCAGCGGCTTTTGCCGGTGCTCTGTC |
| | | AGGCCCACGGGCTCACACCGGAACAG |
| | | GTCGTAGCTATTGCGTCCCATGACGGC |
| | | GGAAAGCAAGCCTTAGAAACTGTGCA |
| | | GCGGTTGCTGCCTGTTCTGTGCCAGGC |
| | | TCATGGGCTCACTCCCGAGCAAGTGGT |
| | | CGCGATTGCGAGCAACGGAGGTGGCA |
| | | AGCAAGCACTAGAAACCGTGCAACGA |
| | | CTGCTGCCGGTCTTGTGCCAAGCGCAT |
| | | GGCCTGACACCCGAACAGGTGGTGGCT |
| | | ATCGCGTCCAACAACGGGGGCAAACA |
| | | GGCCCTGGAAACAGTCCAAAGGCTGTT |
| | | GCCGGTCCTCTGTCAAGCTCACGGACT |
| | | AACCCCCGAACAGGTGGTGGCAATAG |
| | | CTTCGCGGCATGGGGGCAAGCAGGCA |
| | | CTCGAAACAGTGCAGAGGTTGCTCCCG |
| | | GTACTTTGCCAAGCACATGGCTTGACC |
| | | CCTGAGCAAGTCGTGGCCATCGCATCC |
| | | AACGGCGGAGGGAAGCAAGCGCTTGA |
| | | AACTGTCCAACGCCTCCTGCCCGTGTT |
| | | GTGTCAAGCCCATGGCCTTACGCCTGA |
| | | ACAAGTCGTCGCTATTGCAAGTAACGG |
| | | AGGAGGGAGGCCTGCCCTGGAGTCCA |
| | | TTGTCGCCCAACTGTCGCGACCCGATC |
| | | CAGCCCTGGCAGCCCTCACTAACGACC |
| | | ATCTCGTGGCGCTGGCATGCCTCGGTG |
| | | GTAGACCAGCTCTGGACGCCGTGAAG |
| | | AAGGGACTTCCTCATGCCCCCGCACTG |
| | | ATCAAGCGCACTAACCGGAGGATCCCT |
| | | GAAAGAACCTCCCACAGAGTG |
| | | (SEQ ID NO: 415) |
| DBD203 | HMVDLRTLGYSQQQQEKIKP | CACATGGTGGACCTCCGGACCCTGGGA |
| TAL09 | KVRSTVAQHHEALVGHGFTH | TACTCCCAGCAGCAGCAGGAAAAGAT |
| human | AHIVALSQHPAALGTVAVKY | CAAGCCGAAGGTCCGCTCCACTGTGGC |
| | QDMIAALPEATHEAIVGVGKR | TCAACACCACGAAGCCCTTGTGGGACA |
| | GAGARALEALLTVAGELRGPP | CGGCTTCACTCATGCACACATTGTGGC |
| | LQLDTGQLLKIAKRGGVTAVE | ATTGTCCCAACACCCTGCCGCTCTGGG |
| | AVHAWRNALTGAPLNLTPEQ | AACTGTGGCAGTGAAGTACCAGGACA |
| | VVAIASNNGGKQALETVQRL | TGATAGCCGCACTGCCCGAAGCAACAC |
| | LPVLCQAHGLTPEQVVAISN | ACGAGGCCATCGTCGGTGTGGGAAAA |
| | IGGKQALETVQRLLPVLCQAH | AGAGGAGCGGGAGCCAGAGCACTCGA |
| | GLTPEQVVAIASHDGGKQALE | AGCGCTCCTGACAGTGGCGGGAGAAC |
| | TVQRLLPVLCQAHGLTPEQVV | TCCGCGGACCACCTCTGCAGCTGGACA |
| | AIASNGGGKQALETVQRLLPV | CTGGCCAGCTGCTGAAGATTGCAAAAC |
| | LCQAHGLTPEQVVAIASNGGG | GCGGTGGAGTCACCGCTGTGGAGGCG |
| | KQALETVQRLLPVLCQAHGL | GTGCATGCCTGGAGAAATGCCCTGACT |
| | TPEQVVAIASNIGGKQALETV | GGTGCCCCCTTGAACCTGACCCCCGAG |
| | QRLLPVLCQAHGLTPEQVVAI | CAAGTCGTCGCAATTGCTTCGAACAAC |
| | ASNNGGKQALETVQRLLPVL | GGTGGTAAACAGGCTCTCGAAACCGTC |
| | CQAHGLTPEQVVAIASHDGG | CAACGGCTGCTCCCAGTCCTATGCCAA |
| | KQALETVQRLLPVLCQAHGL | GCCCATGGACTTACGCCCGAACAAGTG |
| | TPEQVVAIASNIGGKQALETV | GTTGCTATAGCCTCCAATATCGGTGGG |
| | QRLLPVLCQAHGLTPEQVVAI | AAGCAGGCTCTGGAGACAGTCCAAAG |
| | ASNIGGKQALETVQRLLPVLC | ACTCCTGCCGGTCCTCTGCCAAGCGCA |
| | QAHGLTPEQVVAIASNIGGKQ | CGGACTCACCCCAGAGCAAGTAGTGG |
| | ALETVQRLLPVLCQAHGLTPE | CCATTGCCTCCCACGATGGTGGCAAGC |
| | QVVAIASNIGGKQALETVQRL | AGGCCCTTGAAACAGTCCAGAGACTGT |
| | LPVLCQAHGLTPEQVVAIASH | TACCCGTGCTTTGTCAAGCCCACGGGC |
| | DGGKQALETVQRLLPVLCQA | TCACTCCCGAACAGGTGGTGGCGATTG |
| | HGLTPEQVVAIASHDGGKQA | CCTCTAACGGCGGCGGCAAACAGGCG |
| | LETVQRLLPVLCQAHGLTPQE | CTCGAGACTGTCCAGCGGCTTCTACCG |
| | VVAIASNGGGKQALETVQRL | GTGTTGTGCCAGGCCCATGGTCTGACA |
| | LPVLCQAHGLTPEQVVAIASN | CCCGAACAGGTGGTTGCAATTGCCTCC |
| | NGGKQALETVQRLLPVLCQA | AACGGAGGCGGGAAGCAAGCCCTCGA |
| | IIGLTPEQVVAIASHDGGKQA | AACGGTCCAGCGGTTGTTGCCGGTCCT |
| | LETVQRLLPVLCQAHGLTPEQ | GTGTCAGGCGCACGGGTTGACCCCAGA |
| | VVAIASNGGGRPALESIVAQL | ACAGGTCGTCGCTATTGCCTCAAACAT |
| | SRPDPALAALTNDHLVALACL | CGGTGGTAAGCAGGCGCTGGAAACTG |
| | GGRPALDAVKKGLPHAPALIK | TGCAGCGACTCCTGCCTGTGCTCTGTC |
| | RTNRRIPERTSHRV | AAGCGCACGGGCTTACGCCAGAACAA |
| | (SEQ ID NO: 416) | GTGGTCGCTATCGCGTCCAACAACGGC |
| | | GGCAAGCAAGCTCTGGAAACCGTCCA |
| | | AAGGCTCTTGCCCGTCCTATGTCAGGC |
| | | CCACGGCCTCACCCCTGAACAGGTTGT |
| | | CGCCATCGCCAGCCACGACGGCGGTA |
| | | AGCAAGCCTTGGAAACCGTGCAACGA |
| | | CTGCTTCCCGTGTTGTGTCAGGCTCAT |
| | | GGCCTCACTCCTGAGCAAGTTGTGGCC |
| | | ATCGCTTCGAATATTGGGGGCAAACAA |
| | | GCCCTGGAAACGGTGCAGCGGTTGCTG |
| | | CCTGTCTTGTGTCAAGCTCACGGGCTG |
| | | ACTCCTGAACAAGTCGTGGCCATAGCG |
| | | TCCAACATTGGGGGAAAGCAGGCGTT |
| | | GGAAACTGTCCAGAGGCTGTTGCCCGT |
| | | GCTCTGCCAGGCTCACGGCCTGACCCC |
| | | TGAACAGGTGGTCGCAATCGCCTCCAA |
| | | CATCGGAGGGAAGCAGGCACTGGAAA |
| | | CAGTGCAGCGGCTGCTGCCCGTGCTGT |
| | | GCCAAGCTCATGGACTCACTCCGGAAC |
| | | AGGTCGTTGCAATCGCATCAAACATTG |
| | | GAGGAAAGCAGGCCCTCGAGACAGTG |
| | | CAGAGACTGCTCCCTGTGCTTTGCCAG |
| | | GCGCATGGCCTTACCCCGGAACAAGTG |
| | | GTAGCCATCGCGTCGCATGACGGCGGA |
| | | AAGCAGGCTTTGGAAACCGTGCAAAG |
| | | GCTCCTGCCCGTCCTGTGCCAGGCTCA |
| | | CGGTCTGACCCCGGAACAAGTCGTGGC |
| | | CATCGCTAGCCATGATGGAGGGAAAC |
| | | AGGCCCTGGAGACTGTACAGAGACTCC |
| | | TCCCTGTCCTTTGCCAAGCACACGGAC |
| | | TTACCCCTGAACAGGTCGTGGCTATTG |
| | | CATCGAACGGGGGGGGGAAACAAGCG |
| | | CTCGAAACTGTGCAACGCCTCCTGCCA |
| | | GTGTTGTGCCAGGCACATGGCTTGACT |
| | | CCCGAGCAAGTCGTCGCCATTGCATCA |
| | | AACAACGGAGGAAAACAGGCACTCGA |
| | | GACTGTGCAAAGACTGTTGCCGGTGCT |
| | | ATGCCAGGCACACGGCTTGACCCCGGA |
| | | GCAAGTGGTGGCTATCGCCTCGCACGA |
| | | TGGCGGAAAACAAGCACTCGAAACTG |
| | | TTCAGCGGCTACTGCCGGTGCTCTGCC |
| | | AGGCTCACGGACTGACTCCAGAACAG |
| | | GTGGTCGCAATCGCGTCGAACGGTGGA |
| | | GGACGGCCTGCCCTCGAGAGCATTGTG |
| | | GCGCAGCTCTCCCGCCCGGATCCTGCT |
| | | TTGGCCGCGCTGACCAACGACCACCTG |
| | | GTGGCCCTCGCCTGTTTGGGGGGTCGG |
| | | CCTGCTTTGGACGCCGTCAAGAAGGGC |
| | | CTGCCTCATGCCCCCGCACTCATCAAG |
| | | CGGACCAATAGACGCATCCCGGAAAG |
| | | GACTTCTCACCGGGTG |
| | | (SEQ ID NO: 417) |
| DBD170 | HMVDLRTLGYSQQQQEKIKP | CACATGGTGGACCTCCGGACTCTTGGT |
| TAL08 | KVRSTVAQHHEALVGHGFTH | TACTCCCAACAGCAGCAGGAAAAGAT |
| human | AHIVALSQHPAALGTVAVKY | CAAGCCTAAGGTCCGCTCCACTGTGGC |
| | QDMIAALPEATHEAIVGVKR | CCAACACCACGAGGCCCTTGTGGGACA |
| | GAGARALEALLTVAGELRGPP | TGGATTCACCCACGCCCACATCGTGGC |
| | LQLDTGQLLKIAKRGGVTAVE | GCTGTCGCAGCACCCTGCTGCCTTGGG |
| | AVHAWRNALTGAPLNLTPEQ | GACCGTCGCCGTGAAGTACCAGGACAT |
| | VVAIASNNGGKQALETVQRL | GATCGCCGCCTTACCCGAAGCCACCCA |
| | LPVLCQAHGLTPEQVVAIASR | CGAGGCAATCGTGGGAGTGGGTAAAA |
| | HGGKQALETVQRLLPVLCQA | GGGGTGCCGGTGCAAGAGCTCTGGAG |
| | HGLTPEQVVAIASHDGGKQA | GCCCTTCTCACCGTGGCCGGAGAACTT |
| | LETVQRLLPVLCQAHGLTPEQ | AGAGGGCCTCCACTCCAGCTGGATACC |
| | VVAIASNIGGKQALETVQRLL | GGTCAGCTGTTGAAGATTGCTAAGAGA |
| | PVLCQAHGLTPEQVVAIASHE | GGAGGGGTGACCGCCGTGGAAGCCGT |
| | GGKQALETVQRLLPVLCQAH | GCATGCGTGGCGCAACGCTCTGACTGG |
| | GLTPEQVVAIASNIGGKQALE | AGCCCCACTGAACTTGACCCCCGAACA |
| | TVQRLLPVLCQAHGLTPEQVV | GGTGGTCGCCATCGCCTCCAATAACGG |
| | AIASHDGGKQALETVQRLLPV | GGGCAAACAGGCGCTGGAAACCGTGC |
| | LCQAHGLTPEQVVAIASNGGG | AGAGATTGCTGCCTGTCTTATGCCAAG |
| | KQALETVQRLLPVLCQAHGL | CACACGGACTCACCCCCGAGCAAGTCG |
| | TPEQVVAIASNIGGKQALETV | TGGCTATCGCCTCCAGACACGGCGGGA |
| | QRLLPVLCQAHGLTPEQVVAI | AGCAAGCTCTGGAGACTGTGCAGAGA |
| | ASNNGGKQALETVQRLLPVL | CTGCTACCCGTGTTGTGTCAAGCCCAC |
| | CQAHGLTPEQVVAIASHDGG | GGTCTTACTCCGGAGCAAGTGGTCGCT |
| | KQALETVQRLLPVLCQAHGL | ATTGCCTCGCATGATGGTGGTAAACAT |
| | TPEQVVAIASHDGGKQALETV | GCCCTGGAGACAGTGCAGCGCTTGCTC |
| | QRLLPVLCQAHGLTPEQVVAI | CCTGTGCTGTGTCAGGCACACGGCCTG |
| | ASHDGGKQALETVQRLLPVL | ACACCGGAACAGGTGGTGGCCATCGCT |
| | CQAHGLTPEQVVAIASHDGG | TCGAACATTGGAGGTAAACAGGCATTG |
| | KQALETVQRLLPVLCQAHGL | GAGACAGTCCAGAGGCTCCTGCCGGTC |
| | TPEQVVAIASNIGGKQALETV | CTCTGCCAAGCTCACGGTCTGACTCCT |
| | QRLLPVLCQAHGLTPEQVVAI | GAACAAGTAGTCGCCATTGCATCCCAC |
| | ASHDGGKQALETVQRLLPVL | GATGGCGGTAAGCAGGCTCTTGAAACT |
| | CQAHGLTPEQVVAIASNNGG | GTCCAGAGACTGTTACCTGTGCTCTGT |
| | KQALETVQRLLPVLCQAHGL | CAGGCCCATGGACTGACGCCTGAACA |
| | TPEQVVAIASNGGGRPALESIV | GGTCGTCGCGATTGCGTCAAACATCGG |
| | AQLSRPDPALAALTNDHLVAL | TGGAAAGCAAGCGCTTGAAACGGTCC |
| | ACLGGRPALDAVKKGLPHAP | AGCGCCTACTGCCTGTGTTGTGCCAAG |
| | ALIKRTNRRIPERTSHRV | CGCACGGCCTCACACCAGAACAAGTC |
| | (SEQ ID NO: 418) | GTCGCAATCGCAAGCCATGATGGCGG |
| | | AAAGCAGGCACTAGAAACTGTTCAGC |
| | | GCCTTCTACCCGTCTTGTGCCAGGCTC |
| | | ATGGACTCACTCCCGAACAAGTGGTGG |
| | | CTATTGCGTCCAATGGAGGCGGTAAAC |
| | | AAGCCCTCGAAACCGTCCAGCGGCTTC |
| | | TGCCGGTTCTGTGCCAGGCACATGGCT |
| | | TGACTCCTGAGCAAGTIGTCGCGATTG |
| | | CCTCCAACATTGGGGGCAAGCAAGCA |
| | | CTCGAAACAGTGCAGAGGCTTCTACCA |
| | | GTGCTCTGCCAGGCCCACGGTTTGACC |
| | | CCTGAACAGGTGGTCGCCATCGCCTCC |
| | | AACAACGGCGGAAAACAAGCACTGGA |
| | | AACTGTGCAAAGACTGCTTCCTGTGCT |
| | | GTGTCAGGCGCATGGCCTCACCCCGGA |
| | | ACAAGTGGTGGCCATAGCGTCGCACG |
| | | ACGGAGGCAAGCAGGCCCTTGAAACG |
| | | GTGCAGCGACTGTTGCCCGTCCTTTGT |
| | | CAGGCTCACGGGTTAACCCCAGAACA |
| | | GGTCGTGGCAATCGCCTCTCATGACGG |
| | | GGGGAAGCAGGCCTTGGAGACTGTCC |
| | | AAAGGTTGCTGCCAGTGCTGTGCCAAG |
| | | CCCACGGGCTCACTCCAGAGCAAGTAG |
| | | TGGCGATAGCGAGCCACGACGGCGGG |
| | | AAACAGGCTCTGGAAACCGTACAGAG |
| | | GTTGCTCCCCGTGCTTTGTCAAGCACA |
| | | TGGTCTGACCCCCGAGCAAGTCGTGGC |
| | | CATTGCCAGCCATGACGGAGGGAAAC |
| | | AGGCACTTGAAACCGTCCAAAGGCTGC |
| | | TGCCTGTGCTTTGCCAGGCGCATGGGC |
| | | TTACTCCTGAACAAGTGGTGGCGATTG |
| | | CTTCAAATATCGGTGGCAAACAAGCTT |
| | | TGGAAACAGTCCAGCGGTTGCTCCCAG |
| | | TCCTCTGTCAAGCTCACGGGCTGACGC |
| | | CAGAACAGGTCGTGGCAATTGCGTCAC |
| | | ACGACGGAGGAAAACAGGCGTTGGAA |
| | | ACCGTTCAGCGGCTCTTGCCCGTGCTA |
| | | TGCCAGGCCCACGGACTGACTCCGGAA |
| | | CAGGTGGTTGCCATCGCCAGCAACAAC |
| | | GGAGGGAAGCAGGCGCTCGAGACTGT |
| | | ACAGCGACTTCTCCCCGTCCTGTGTCA |
| | | AGCCCATGGCCTGACGCCGGAACAGG |
| | | TGGTAGCCATTGCGAGCAACGGAGGA |
| | | GGACGCCCAGCCCTAGAGAGCATCGT |
| | | GGCCCAACTGTCGCGGCCGGATCCAGC |
| | | TCTGGCCGCATTGACCAACGACCACCT |
| | | GGTCGCTCTGGCTTGTCTGGGAGGACG |
| | | ACCGGCCCTGGATGCCGTGAAGAAGG |
| | | GCCTGCCACATGCCCCTGCCCTCATTA |
| | | AGCGGACTAACAGAAGGATCCCCGAG |
| | | AGGACCTCACACAGAGTC |
| | | (SEQ ID NO: 419) |
| DBD088 | HMVDLRTLGYSQQQQEKIKP | CACATGGTGGACCTGAGAACCCTGGG |
| TAL04 | KVRSTVAQHHEALVGHGFTH | ATACTCGCAGCAGCAGCAGGAAAAGA |
| human | AHIVALSQHPAALGTVAVKY | TCAAGCCCAAAGTGCGGTCCACTGTGG |
| | QDMIAALPEATHEAIVGVGKR | CCCAGCACCATGAGGCCCTCGTCGGAC |
| | GAGARALEALLTVAGELRGPP | ACGGATTCACCCACGCGCACATCGTGG |
| | LQLDTGQLLKIAKRGGVTAVE | CTCTGTCACAGCATCCCGCCGCCTTGG |
| | AVHAWRNALTGAPLNLTPEQ | GAACCGTGGCTGTGAAGTACCAGGAC |
| | VVAIASNNGGKQALETVQRL | ATGATTGCTGCACTTCCTGAGGCGACC |
| | LPVLCQAHGLTPEQVVAIASH | CACGAAGCCATTGTGGGAGTCGGAAA |
| | DGGKQALETVQRLLPVLCQA | ACGGGGTGCAGGCGCTAGAGCCCTGG |
| | HGLTPEQVVAIASNIGGKQAL | AGGCCCTGTTAACGGTGGCAGGCGAA |
| | ETVQRLLPVLCQAHGLTPEQV | CTGCGGGGACCACCATTGCAGCTCGAT |
| | VAIASNIGGKQALETVQRLLP | ACCGGCCAGCTGCTGAAGATTGCCAAG |
| | VLCQAHGLTPEQVVAIASHDG | AGGGGCGGAGTCACAGCGGTGGAGGC |
| | GKQALETVQRLLPVLCQAHG | GGTCCATGCCTGGCGCAACGCTCTTAC |
| | LTPEQVVAIASNNGGKQALET | TGGGGCTCCACTGAACCTGACCCCTGA |
| | VQRLLPVLCQAHGLTPEQVV | ACAGGTAGTGGCCATCGCATCGAACA |
| | AIASNIGGKQALETVQRLLPV | ATGGCGGTAAGCAGGCTCTCGAAACC |
| | LCQAHGLTPEQVVAIASNGGG | GTCCAAAGGCTCCTTCCGGTGTTATGC |
| | KQALETVQRLLPVLCQAHGL | CAGGCTCATGGGCTGACGCCTGAACAA |
| | TPEQVVAIASHDGGKQALETV | GTGGTCGCAATCGCATCGCATGACGGG |
| | QRLLPVLCQAHGLTPEQVVAI | GGGAAACAGGCATTAGAAACCGTGCA |
| | ASNIGGKQALETVQRLLPVLC | ACGCCTCCTTCCTGTCCTTTGTCAAGCA |
| | QAHGLTPEQVVAIASNIGGKQ | CATGGATTGACTCCTGAGCAAGTTGTC |
| | ALETVQRLLPVLCQAHGLTPE | GCGATTGCCTCCAACATTGGCGGCAAA |
| | QVVAIASNGGGKQALETVQR | CAGGCACTGGAAACCGTTCAACGGCTC |
| | LLPVLCQAHGLTPEQVVAIAS | CTCCCCGTACTGTGCCAGGCACACGGC |
| | NNGGKQALETVQRLLPVLCQ | CTGACCCCCGAACAAGTCGTCGCCATT |
| | AHGLTPEQVVAIASRHGGKQ | GCCTCGAACATCGGAGGCAAGCAGGC |
| | ALETVQRLLPVLCQAHGLTPE | GCTTGAAACCGTGCAGCGCTTGCTGCC |
| | QVVAIASRHGGKQALETVQR | TGTGCTCTGCCAAGCGCACGGCCTAAC |
| | LLPVLCQAHGLTPEQVVAIAS | CCCTGAACAGGTGGTAGCAATTGCTTC |
| | NIGGKQALETVQRLLPVLCQA | CCACGACGGTGGGAAGCAGGCCCTCG |
| | HGLTPEQVVAIASNGGGKQA | AGACAGTGCAAAGACTGTTGCCGGTCC |
| | LETVQRLLPVLCQAHGLTPEQ | TCTGCCAGGCCCACGGACTTACCCCCG |
| | VVAIASNGGGRPALESIVAQL | AGCAAGTCGTGGCTATTGCGTCCAATA |
| | SRPDPALAALTNDHLVALACL | ATGGCGGAAAACAGGCACTTGAGACA |
| | GGRPALDAVKKGLPHAPALIK | GTCCAGAGGCTGCTGCCTGTCTTATGC |
| | RTNRRIPERTSHRV | CAAGCCCACGGTCTTACTCCAGAACAG |
| | (SEQ ID NO: 420) | GTGGTCGCTATCGCATCAAACATTGGA |
| | | GGAAAACAGGCGCTGGAAACTGTCCA |
| | | ACGCCTGTTGCCCGTGCTCTGTCAGGC |
| | | TCACGGGCTGACTCCCGAGCAAGTGGT |
| | | GGCAATCGCCTCCAACGGGGGAGGAA |
| | | AGCAGGCCTTGGAAACCGTACAGCGG |
| | | CTGTTGCCTGTGTTGTGCCAAGCTCAC |
| | | GGTCTGACGCCAGAGCAAGTGGTGGC |
| | | CATTGCTTCGCACGACGGCGGGAAGCA |
| | | GGCACTCGAAACGGTGCAGAGGTTGCT |
| | | TCCCGTCTTGTGCCAGGCGCACGGACT |
| | | CACCCCAGAACAAGTGGTCGCAATTGC |
| | | TAGCAACATTGGTGGCAAACAAGCGCT |
| | | CGAAACTGTACAACGGCTGCTCCCGGT |
| | | GCTTTGCCAAGCCCACGGGCTTACCCC |
| | | AGAGCAAGTGGTGGCCATTGCGAGCA |
| | | ACATCGGGGGCAAGCAAGCGCTTGAG |
| | | ACTGTACAAAGACTGCTGCCCGTGCTG |
| | | TGCCAAGCCCACGGCTTAACCCCGGAA |
| | | CAGGTCGTGGCCATCGCCTCGAATGGA |
| | | GGTGGAAAGCAAGCCCTGGAGACTGT |
| | | GCAGCGGTTGCTGCCCGTTCTGTGTCA |
| | | AGCTCACGGACTGACACCGGAACAGG |
| | | TGGTGGCTATCGCAAGCAACAACGGC |
| | | GGAAAGCAAGCCCTGGAGACTGTGCA |
| | | GCGGCTGCTGCCAGTGCTGTGTCAGGC |
| | | GCATGGACTGACCCCCGAACAAGTCGT |
| | | AGCCATAGCCAGCCGCCACGGAGGGA |
| | | AGCAGGCCCTGGAAACAGTCCAGCGC |
| | | CTCTTGCCCGTGTTGTGTCAAGCGCAT |
| | | GGCCTCACCCCTGAACAGGTCGTTGCC |
| | | ATTGCGTCCAGACATGGAGGGAAGCA |
| | | AGCACTAGAAACAGTGCAGAGACTGC |
| | | TACCCGTCCTGTGCCAGGCCCATGGAC |
| | | TCACGCCGGAGCAAGTGGTGGCGATA |
| | | GCATCGAACATTGGTGGGAAACAAGC |
| | | CCTTGAAACTGTCCAGAGACTGTTACC |
| | | TGTGCTTTGTCAGGCACATGGGCTCAC |
| | | ACCAGAACAGGTGGTCGCCATCGCTAG |
| | | CAACGGAGGGGGAAAACAGGCTTTGG |
| | | AGACTGTCCAGCGGCTCCTACCGGTGC |
| | | TCTGCCAAGCACACGGATTGACGCCCG |
| | | AACAGGTTGTGGCGATTGCGTCCAACG |
| | | GTGGAGGACGGCCTGCCCTGGAGAGC |
| | | ATTGTGGCTCAGCTCTCCAGACCTGAC |
| | | CCAGCCCTCGCAGCTCTGACTAACGAT |
| | | CATCTGGTGGCGCTGGCCTGTTTGGGG |
| | | GGACGCCCTGCTTTGGACGCCGTGAAG |
| | | AAGGGTCTGCCACATGCCCCCGCACTG |
| | | ATCAAGCGCACCAACCGGCGAATCCCT |
| | | GAGCGCACTTCCCATCGAGTG |
| | | (SEQ ID NO: 421) |

In some embodiments, the site-specific targeting moieties of the invention target a sequence within a CXCL9, CXCL10, and/or CXCL promoter region. In some embodiments, the site-specific targeting moieties of the invention target a sequence within a CXCL9, CXCL10, and/or CXCL enhancer region. In some embodiments, the site-specific targeting moieties of the invention taregt a sequence within a CXCL9, CXCL10, and/or CXCL anchor region.

In some embodiments, the site-specific targeting moieties of the invention target a sequence within genomic coordinates provided in Table 6.

TABLE 6
Target regions (human)

| | Expression | Genomic |
|---|---|---|
| Description | Control Region | Coordinates |
| CXCL9 Promoter | Promoter | chr4: 76928543-76931536 |
| CXCL10 Promoter | Promoter | chr4: 76944524-76948668 |
| CXCL11 Promoter | Promoter | chr4: 76957098-76962383 |
| CXCL10-11 Enhancer | cRE | chr4: 76949523-76949873 |
| CXCL9-10 Enhancer | cRE | chr4: 76932906-76933929 |
| CTCF “L1” | Anchor | chr4: 76916900-76917300 |
| CTCF “R2” | Anchor | chr4: 76988761-76989260 |
| CTCF “R3” | Anchor | chr4: 77008274-77008727 |
| CTCF “R1” | Anchor | chr4: 76957852-76958551 |

TABLE 7
Target Regions (mouse)

| | Expression Control | |
|---|---|---|
| Description | Region | Genomic Coordinates |
| Cxcl9 Promoter | Promoter | chr5: 92327958-92329175 |
| Cxcl10 Promoter | Promoter | chr5: 92348741-92350744 |
| Cxcl11 Promoter | Promoter | chr5: 92363149-92363868 |
| Enhancer: 10-11 | cRE | chr5: 92352272-92354744 |

In some embodiments, the site-specific targeting moieties of the invention target a sequence within genomic coordinates chr4: 76928543-76931536. In some embodiments, the site-specific targeting moieties of the invention target a sequence within genomic coordinates chr4: 76944524-76948668. In some embodiments, the site-specific targeting moieties of the invention target a sequence within genomic coordinates chr4: 76957098-76962383. In some embodiments, the site-specific targeting moieties of the invention target a sequence within genomic coordinates chr4: 76949523-76949873. In some embodiments, the site-specific targeting moieties of the invention target a sequence within genomic coordinates chr4: 76932906-76933929. In some embodiments, the site-specific targeting moieties of the invention target a sequence within genomic coordinates chr4: 76916900-76917300. In some embodiments, the site-specific targeting moieties of the invention target a sequence within genomic coordinates chr4: 76988761-76989260. In some embodiments, the site-specific targeting moieties of the invention target a sequence within genomic coordinates chr4: 77008274-77008727. In some embodiments, the site-specific targeting moieties of the invention target a sequence within genomic coordinates chr4: 76957852-76958551.

In some embodiments, the site-specific targeting moieties of the invention target a sequence within a CXCL9, CXCL10, and/or CXCL11 promoter, enhancer or anchor region. Exemplary target sequences provided in Table 8 corresponding with zinc finger DNA binding domains in Table 3.

TABLE 8

| | | Genomic Coordinates of the Target Start |
|---|---|---|
| Exemplary | | Site in |
| Zinc Finger | | Genome Reference Consortium Human |
| DBD | Target Sequence | Build 37 (GRCh37): Chromosome 4) |
| DBD386 | AGCCATGACACTGCACTACCC | chr4: 76958201-76958221 (negative strand) |
| ZF24 | (SEQ ID NO: 422) | |
| DBD374 | ATTCTGCACCAGCTGAGGGGA | chr4: 76949758-76949778 (negative strand) |
| ZF16 | (SEQ ID NO: 423) | |
| DBD360 | GATCCAAATGCTAATGTAACC | chr4: 76933245-76933265 (positive strand) |
| ZF07 | (SEQ ID NO: 424) | |
| DBD358 | GCATGACAAGTTCCTAAACGA | chr4: 76932967-76932987 (negative strand) |
| ZF05 | (SEQ ID NO: 425) | |

In some embodiments, the site-specific targeting moieties of the invention target a sequence within a CXCL9, CXCL10, and/or CXCL11 promoter, enhancer or anchor region. Exemplary target sequences provided in Tables 9 and 10 corresponding with TALE DNA binding domains in Tables 4 and 5, respectively.

TABLE 9

| | | Genomic Coordinates of the Target |
|---|---|---|
| Exemplary | | Start Site in |
| TALE DBD | | Genome Reference Consortium Mouse |
| mouse | Target Sequence | Build 37 (GRCh37): Chromosome 5) |
| DBD455 | GATATCTAAGTCAGGTTC | chr5: 92349207-92349224 (positive |
| TAL37 | (SEQ ID NO: 426) | strand) |
| mouse | | |
| DBD452 | ATCCCTTACTATAAACTC | chr5: 92328266-92328283 (negative |
| TAL34 | (SEQ ID NO: 427) | strand) |
| mouse | | |
| DBD429 | AAGATAGATGAAGGTCAC | chr5: 92363632-92363649 (negative |
| TAL27 | (SEQ ID NO: 428) | strand) |
| mouse | | |

TABLE 10

| | | Genomic Coordinates of the Target |
|---|---|---|
| Exemplary | | Start Site in |
| TALE DBD | | Genome Reference Consortium Human |
| human | Target Sequence | Build 37 (GRCh37): Chromosome 4) |
| DBD330 | GAAGGGCATGGCTATAGC | chr4: 76957115-76957132 (negative strand) |
| TAL14 | (SEQ ID NO: 429) | |
| human | | |
| DBD203 | GACTTAGCAAAACCTGCT | chr4: 76944734-76944751 (positive strand) |
| TAL09 | (SEQ ID NO: 430) | |
| human | | |
| DBD170 | GGCACACTAGCCCCACGT | chr4: 76944656-76944673 (negative strand) |
| TAL08 | (SEQ ID NO: 431) | |
| human | | |
| DBD088 | GCAACGATCAATGGGATT | chr4: 76933040-76933057 (negative strand) |
| TAL04 | (SEQ ID NO: 432) | |
| human | | |

In some embodiments, the site-specific targeting moieties target a genomic sequence located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb away from chr4: 76958201-76958221. In some embodiments, the site-specific targeting moieties of the invention target genomic sequence chr4: 76958201-76958221.

In some embodiments, the site-specific targeting moieties target a genomic sequence located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb away from chr4: 76949758-76949778. In some embodiments, the site-specific targeting moieties of the invention target genomic sequence chr4: 76949758-76949778.

In some embodiments, the site-specific targeting moieties target a genomic sequence located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb away from chr4: 76933245-76933265. In some embodiments, the site-specific targeting moieties of the invention target genomic sequence chr4: 76933245-76933265.

In some embodiments, the site-specific targeting moieties target a genomic sequence located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb away from chr4: 76932967-76932987. In some embodiments, the site-specific targeting moieties of the invention target genomic sequence chr4: 76932967-76932987.

In some embodiments, the site-specific targeting moieties target a genomic sequence located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb away from chr4: 76957115-76957132. In some embodiments, the site-specific targeting moieties of the invention target genomic sequence chr4: 76957115-76957132.

In some embodiments, the site-specific targeting moieties target a genomic sequence located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb away from chr4: 76944734-76944751. In some embodiments, the site-specific targeting moieties of the invention target genomic sequence chr4: 76944734-76944751.

In some embodiments, the site-specific targeting moieties target a genomic sequence located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb away from chr4: 76944656-76944673. In some embodiments, the site-specific targeting moieties of the invention target genomic sequence chr4: 76944656-76944673.

In some embodiments, the site-specific targeting moieties target a genomic sequence located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb away from chr4: 76933040-76933057. In some embodiments, the site-specific targeting moieties of the invention target genomic sequence chr4: 76933040-76933057.

In some embodiments, the site-specific targeting moieties of the invention comprising a polynucleotide comprise a guide RNA (or gRNA) or nucleic acid encoding a guide RNA. A gRNA is a short synthetic RNA molecule comprising a “scaffold” sequence necessary for, e.g., directing an effector to an expression control element associated with CXCL9, CXCL10, and/or CXCL11 which may, e.g., include an about 20 nucleotide site-specific sequence targeting a genomic target sequence comprising the expression control element associated with CXCL9, CXCL10, and/or CXCL11.

Generally, guide RNA sequences are designed to have a length of between about 17 to about 24 nucleotides (e.g., 19, 20, or 21 nucleotides) and are complementary to the target sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgNAs have also been demonstrated to be effective in genome editing; see, for example, Hendel et al. (2015) Nature Biotechnol., 985-991.

In certain embodiments, the site-specific targeting moieties of the invention comprise a guide RNA (or gRNA) or nucleic acid encoding a guide RNA and a protein or a peptide. In some embodiment, the protein or the peptide comprises a CRISPR associated protein (Cas) polypeptide, or fragment thereof (e.g., a Cas9 polypeptide, or fragment thereof). In one embodiment, a suitable Cas polypeptide is an enzymatically inactive Cas polypeptide, e.g., a “dead Cas polypeptide” or “dCas” polypeptide.

It will be understood that, although the sequences in Tables 2 and 6 are described as modified (or unmodified), the nucleic acid molecules encompassed by the of the invention, e.g., a site-specific epigenetic modifying agent, may comprise any one of the sequences set forth in any one of Tables 2 or 6 that is unmodified or modified differently than described therein. It will also be understood that although some of the sequences in Table 6 have “Ts”, when used as an RNA molecule, such as a guide RNA, in the site-specific targeting moieties of the invention, the “Ts” may be replaced with “Us.”

In some embodiments, a site-specific targeting moiety comprising a polynucleotide, e.g., gRNA, comprises a nucleotide sequence complementary to an anchor sequence. In one embodiment, the anchor sequence comprises a CTCF-binding motif or consensus sequence:

N(T/C/G)N(G/A/T)CC(A/T/G)(C/G)(C/T/A)AG(G/A)(G/T)
GG(C/A/T)(G/A)(C/G)(C/T/A)(G/A/C),

where N is any nucleotide. A CTCF-binding motif or consensus sequence may also be in the opposite orientation, e.g.,

(G/A/C)(C/T/A)(C/G)(G/A)(C/A/T)GG(G/T)(G/A)GA
(C/T/A)(C/G)(A/T/G)CC(G/A/T)N(T/C/G)N.

In some embodiments, the nucleic acid sequence comprises a sequence complementary to a CTCF-binding motif or consensus sequence.

In some embodiments, the polynucleotide comprises a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary to an anchor sequence.

In some embodiments, the polynucleotide comprises a nucleotide sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary to a CTCF-binding motif or consensus sequence. In some embodiments, the polynucleotide is selected from the group consisting of a gRNA, and a sequence complementary or a sequence comprising at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary sequence to an anchor sequence.

In some embodiments, a site-specific targeting moiety comprising a polynucleotide of the invention is an RNAi molecule. RNAi molecules comprise RNA or RNA-like structures typically containing 15-50 base pairs (such as about 8-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599, 8,349,809, and 8,513,207). In one embodiment, the invention includes a composition to inhibit expression of a gene encoding a polypeptide described herein, e.g., a conjunction nucleating molecule.

RNAi molecules comprise a sequence substantially complementary, or fully complementary, to all or a fragment of a target gene. RNAi molecules may complement sequences at the boundary between introns and exons to prevent the maturation of newly-generated nuclear RNA transcripts of specific genes into mRNA for transcription. RNAi molecules complementary to specific genes can hybridize with the mRNA for that gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (R G).

RNAi molecules can be provided to the cell as “ready-to-use” RNA synthesized in vitro or as an antisense gene transfected into cells which will yield RNAi molecules upon transcription. Hybridization with mRNA results in degradation of the hybridized molecule by RNAse H and/or inhibition of the formation of translation complexes. Both result in a failure to produce the product of the original gene.

The length of the RNAi molecule that hybridizes to the transcript of interest should be around 10 nucleotides, between about 15 or 30 nucleotides, or about 15, 16, 17, 18, 19, 20, 21, 22, 23, 24, 25, 26, 27, 28, 29, 30 or more nucleotides. The degree of identity of the antisense sequence to the targeted transcript should be at least 75%, at least 80%, at least 85%, at least 90%, or at least 95.

RNAi molecules may also comprise overhangs, i.e. typically unpaired, overhanging nucleotides which are not directly involved in the double helical structure normally formed by the core sequences of the herein defined pair of sense strand and antisense strand. RNAi molecules may contain 3′ and/or 5′ overhangs of about 1-5 bases independently on each of the sense strands and antisense strands. In one embodiment, both the sense strand and the antisense strand contain 3′ and 5′ overhangs. In one embodiment, one or more of the 3′ overhang nucleotides of one strand base pairs with one or more 5′ overhang nucleotides of the other strand. In another embodiment, the one or more of the 3′ overhang nucleotides of one strand base do not pair with the one or more 5′ overhang nucleotides of the other strand. The sense and antisense strands of an RNAi molecule may or may not contain the same number of nucleotide bases. The antisense and sense strands may form a duplex wherein the 5′ end only has a blunt end, the 3′ end only has a blunt end, both the 5′ and 3′ ends are blunt ended, or neither the 5′ end nor the 3′ end are blunt ended. In another embodiment, one or more of the nucleotides in the overhang contains a thiophosphate, phosphorothioate, deoxynucleotide inverted (3′ to 3′ linked) nucleotide or is a modified ribonucleotide or deoxynucleotide.

Small interfering RNA (siRNA) molecules comprise a nucleotide sequence that is identical to about 15 to about 25 contiguous nucleotides of the target mRNA. In some embodiments, the siRNA sequence commences with the dinucleotide AA, comprises a GC-content of about 30-70% (about 50-60%, about 40-60%, or about 45%-55%), and does not have a high percentage identity to any nucleotide sequence other than the target in the genome of the mammal in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl: S8-13, 2006; Lim et al, Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Perm Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

An RNAi molecule modulates expression of RNA encoded by a gene. Because multiple genes can share some degree of sequence homology with each other, in some embodiments, the RNAi molecule can be designed to target a class of genes with sufficient sequence homology. In some embodiments, the RNAi molecule can contain a sequence that has complementarity to sequences that are shared amongst different gene targets or are unique for a specific gene target. In some embodiments, the RNAi molecule can be designed to target conserved regions of an RNA sequence having homology between several genes thereby targeting several genes in a gene family (e.g., different gene isoforms, splice variants, mutant genes, etc.). In some embodiments, the RNAi molecule can be designed to target a sequence that is unique to a specific RNA sequence of a single gene.

In some embodiments, the RNAi molecule targets a sequence in a conjunction nucleating molecule, e.g., CTCF, cohesin, USF 1, YY1, TATA-box binding protein associated factor 3 (TAF3), ZNF 143, or another polypeptide that promotes the formation of an anchor sequence-mediated conjunction, or an epigenetic modifying agent, e.g., an enzyme involved in post-translational modifications including, but are not limited to, DNA methylases (e.g., DNMT3a, DNMT3b, DNMTL), histone methyltransferases, histone deacetylase (e.g., HDACI, HDAC2, HDAC3), sirtuin 1, 2, 3, 4, 5, 6, or 7, lysine-specific histone demethylase 1 (LSD1), histone-lysine-N-methyltransferase (Setdbl), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), protein-lysine N-methyltransferase (SMYD2), and others. In one embodiment, the RNAi molecule targets a protein deacetylase, e.g., sirtuin 1, 2, 3, 4, 5, 6, or 7. In one embodiment, the invention includes a composition comprising an RNAi that targets a conjunction nucleating molecule, e.g., CTCF.

In some embodiments, the site-specific targeting moiety comprises a peptide or protein moiety. In some embodiments, a site-specific epigenetic modifying agent comprises a fusion protein. In some embodiments, an effector is a peptide or protein moiety. The peptide or protein moieties may include, but is not limited to, a peptide ligand, antibody fragment, or targeting aptamer that binds a receptor such as an extracellular receptor, neuropeptide, hormone peptide, peptide drug, toxic peptide, viral or microbial peptide, synthetic peptide, and agonist or antagonist peptide.

Exemplary peptides or protein include a DNA-binding protein, a CRISPR component protein, a conjunction nucleating molecule, a dominant negative conjunction nucleating molecule, an epigenetic modifying agent, or any combination thereof. In some embodiments, the peptide comprises a nuclease, a physical blocker, an epigenetic recruiter, and an epigenetic CpG modifier, and fragments and combinations of any of the foregoing. In some embodiments, the peptide comprises a DNA-binding domain of a protein, such as a helix-turn-helix motif, a leucine zipper, a Zn-finger, a TATA box binding proteins, a transcription factor.

Peptides or proteins may be linear or branched. The peptide or protein moiety may have a length from about 5 to about 200 amino acids, about 15 to about 150 amino acids, about 20 to about 125 amino acids, about 25 to about 100 amino acids, about 20 to about 70 amino acids, about 20 to about 80 amino acids, about 20 to about 90 amino acids, about 30 to about 100 amino acids, about 30 to about 60 amino acids, about 30 to about 80 amino acids, about 35 to about 85 amino acids, about 40 to about 100 amino acids, or about 50 to about 125 amino acids or any range there between.

As indicated above, in some embodiments, the site-specific targeting moieties of the invention comprise a fusion protein.

In some embodiments, the fusion proteins of the invention include a site-specific targeting moiety which targets an expression control region associated with CXCL9, CXCL10, and/or CXCL11 and an effector molecule. In other embodiments, a fusion protein of the invention comprises an effector molecule. Exemplary effector molecules are described below and in some embodiments include, for example, nucleases, physical blockers, epigenetic recruiters, e.g., a transcriptional repressor and epigenetic CpG modifiers, e.g., a DNA methylase, a histone modifying agent, or a histone deacetylase, and combinations of any of the foregoing.

For example, a site-specific targeting moiety may comprise a gRNA and an effector, such as a nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a target sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate a DNA sequence).

In one embodiment, a fusion protein of the invention may comprise an effector molecule comprising, for example, a CRISPR associated protein (Cas) polypeptide, or fragment thereof, (e.g., a Cas9 polypeptide, or fragment thereof) and an epigenetic recruiter or an epigenetic CpG modifier.

In one embodiment, a suitable Cas polypeptide is an enzymatically inactive Cas polypeptide, e.g., a “dead Cas polypeptide” or “dCas” polypeptide.

Exemplary Cas polypeptides that are adaptable to the methods and compositions described herein are described below. Using methods known in the art, a Cas polypeptide can be fused to any of a variety of agents and/or molecules as described herein; such resulting fusion molecules can be useful in various disclosed methods.

In one aspect, the invention includes a composition comprising a protein comprising a domain, e.g., an effector, that acts on DNA (e.g., a nuclease domain, e.g., a Cas9 domain, e.g., a dCas9 domain; a DNA methyltransferase, a deaminase), in combination with at least one guide RNA (gRNA) or antisense DNA oligonucleotide that targets the protein to site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a methylase domain.

In one aspect, the invention includes a composition comprising a protein comprising a domain, e.g., an effector, that comprises a transcriptional control element (e.g., a nuclease domain, e.g., a Cas9 domain, e.g., a dCas9 domain; a transcriptional repressor), in combination with at least one guide RNA (gRNA) or antisense DNA oligonucleotide that targets the protein to a site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a transcriptional repressor domain.

As used herein, a “biologically active portion of an effector domain” is a portion that maintains the function (e.g. completely, partially, minimally) of an effector domain (e.g., a “minimal” or “core” domain).

In some aspects, a linker comprises 2 or more amino acids, e.g., one or more GS sequences. In some aspects, fusion of Cas9 (e.g., dCas9) with two or more effector domains (e.g., of a DNA methylase or deacetylase) comprises one or more interspersed linkers (e.g., GS linkers) between the domains. In some aspects, dCas9 is fused with 2-5 effector domains with interspersed linkers.

In some embodiments, a site-specific targeting moiety comprises a conjunction nucleating molecule, a nucleic acid encoding a conjunction nucleating molecule, or a combination thereof. In some embodiments, an anchor sequence-mediated conjunction is mediated by a first conjunction nucleating molecule bound to the first anchor sequence, a second conjunction nucleating molecule bound to the noncontiguous second anchor sequence, and an association between the first and second conjunction nucleating molecules. In some embodiments, a conjunction nucleating molecule may disrupt, e.g., by competitive binding, the binding of an endogenous conjunction nucleating molecule to its binding site.

The conjunction nucleating molecule may be, e.g., CTCF, cohesin, USF1, YY1, TATA-box binding protein associated factor 3 (TAF3), ZNF143 (Zinc Finger Protein 143) binding motif, or another polypeptide that promotes the formation of an anchor sequence-mediated conjunction. The conjunction nucleating molecule may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. The conjunction nucleating molecule may modulate DNA interactions within or around the anchor sequence-mediated conjunction. For example, the conjunction nucleating molecule can recruit other factors to the anchor sequence that alters an anchor sequence-mediated conjunction formation or disruption.

The conjunction nucleating molecule may also have a dimerization domain for homo- or heterodimerization. One or more conjunction nucleating molecules, e.g., endogenous and engineered, may interact to form the anchor sequence-mediated conjunction. In some embodiments, the conjunction nucleating molecule is engineered to further include a stabilization domain, e.g., cohesion interaction domain, to stabilize the anchor sequence-mediated conjunction. In some embodiments, the conjunction nucleating molecule is engineered to bind a target sequence, e.g., target sequence binding affinity is modulated. In some embodiments, the conjunction nucleating molecule is selected or engineered with a selected binding affinity for an anchor sequence within the anchor sequence-mediated conjunction. Conjunction nucleating molecules and their corresponding anchor sequences may be identified through the use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include ChlA-PET analysis using a bait, such as Cohesin, YY1 or USF1, ZNF143 binding motif, and MS to identify complexes that are associated with the bait.

### Effector Molecules

Effector molecules for use in the compositions and methods of the invention include those that modulate a biological activity, for example increasing or decreasing enzymatic activity, gene expression, cell signaling, and cellular or organ function. Preferred effector molecules of the invention are nucleases, physical blockers, epigenetic recruiters, e.g., a transcriptional repressor and epigenetic CpG modifiers, e.g., a DNA methylase, a histone modifying agent, or a histone deacetylase, and combinations of any of the foregoing.

Additional effector activities may also include binding regulatory proteins to modulate activity of the regulator, such as transcription or translation. Effector molecules also may include activator or inhibitor (or “negative effector”) functions as described herein. In another example, the effector molecule may inhibit substrate binding to a receptor and inhibit its activation, e.g., naltrexone and naloxone bind opioid receptors without activating them and block the receptors' ability to bind opioids. Effector molecules may also modulate protein stability/degradation and/or transcript stability/degradation. For example, proteins may be targeted for degradation by the polypeptide co-factor, ubiquitin, onto proteins to mark them for degradation. In another example, an effector molecule inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the effector molecule is a chemical, e.g., a chemical that modulates a cytosine (C) or an adenine (A) (e.g., Na bisulfite, ammonium bisulfite). In some embodiments, the effector molecule has enzymatic activity (methyl transferase, demethylase, nuclease (e.g., Cas9), a deaminase). In some embodiments, the effector molecule sterically hinders formation of an anchor sequence-mediated conjunction or binding of an RNA polymerase to a promoter.

The effector molecule with effector activity may be any one of the small molecules, peptides, fusion proteins, nucleic acids, nanoparticle, aptamers, or pharmacoagents with poor PK/PD described herein.

In some embodiments, the effector molecule is an inhibitor or “negative effector molecule”. In the context of a negative effector molecule that modulates formation of an anchor sequence-mediated conjunction, in some embodiments, the negative effector molecule is characterized in that dimerization of an endogenous nucleating polypeptide is reduced when the negative effector molecule is present as compared with when it is absent. For example, in some embodiments, the negative effector molecule is or comprises a variant of the endogenous nucleating polypeptide's dimerization domain, or a dimerizing portion thereof.

For example, in certain embodiments, an anchor sequence-mediated conjunction is altered (e.g., disrupted) by use of a dominant negative effector, e.g., a protein that recognizes and binds an anchor sequence, (e.g., a CTCF binding motif), but with an inactive (e.g., mutated) dimerization domain, e.g., a dimerization domain that is unable to form a functional anchor sequence-mediated conjunction. For example, the Zinc Finger domain of CTCF can be altered so that it binds a specific anchor sequence (by adding zinc fingers that recognize flanking nucleic acids), while the homo-dimerization domain is altered to prevent the interaction between the engineered CTCF and endogenous forms of CTCF.

In some embodiments, the effector molecule comprises a synthetic conjunction nucleating molecule with a selected binding affinity for an anchor sequence within a target anchor sequence-mediated conjunction, (the binding affinity may be at least 10%, 15%, 20%, 25%, 30%, 35%, 40%, 45%, 50%, 55%, 60%, 65%, 70%, 75%, 80%, 85%, 90%, 95%, or higher or lower than the affinity of an endogenous conjunction nucleating molecule that associates with the target anchor sequence. The synthetic conjunction nucleating molecule may have between 30-90%, 30-85%, 30-80%, 30-70%, 50-80%, 50-90% amino acid sequence identity to the endogenous conjunction nucleating molecule). The conjunction nucleating molecule may disrupt, such as through competitive binding, the binding of an endogenous conjunction nucleating molecule to its anchor sequence. In some more embodiments, the conjunction nucleating molecule is engineered to bind a novel anchor sequence within the anchor sequence-mediated conjunction.

In some embodiments, a dominant negative effector molecule has a domain that recognizes specific DNA sequences (e.g., an anchor sequence, a CTCF anchor sequence, flanked by sequences that confer sequence specificity), and a second domain that provides a steric presence in the vicinity of the anchoring sequence. The second domain may include a dominant negative conjunction nucleating molecule or fragment thereof, a polypeptide that interferes with conjunction nucleating molecule sequence recognition (e.g., the amino acid backbone of a peptide/nucleic acid or PNA), a nucleic acid sequence ligated to a small molecule that imparts steric interference, or any other combination of DNA recognition elements and a steric blocker.

In some embodiments, the effector molecule is an epigenetic modifying agent. Epigenetic modifying agents useful in the methods and compositions described herein include agents that affect, e.g., DNA methylation, histone deacetylation, and RNA-associated silencing. In some embodiments, the effectors sequence-specifically target an epigenetic enzyme (e.g., an enzyme that generates or removes epigenetic marks, e.g., acetylation and/or methylation). Exemplary epigenetic effectors may target an expression control region comprising, e.g., a transcriptional control element or an anchor sequence, by a site-specific epigenetic modifying agent comprising a site-specific targeting moiety.

In some embodiments, an effector molecule comprises one or more components of a gene editing system. Components of gene editing systems may be used in a variety of contexts including but not limited to gene editing. For example, such components may be used to target agents that physically modify, genetically modify, and/or epigenetically modify CXCL9, CXCL10, and/or CXCL11 sequences.

Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7 (2013): 397-405; CRISPR methods of gene editing are described, e.g., in Guan et al, Application of CRISPR-Cas system in gene therapy: Pre-clinical progress in animal model. DNA Repair 2016 Jul. 30 [Epub ahead of print]; Zheng et al, Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e. g., 5′-NGG, and perform blunt-end cleaving of the target DNA at a location 3 nucleotides upstream from (5′from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf 1-associated CRISPR arrays are processed into mature crRNAs without the requirement of a tracrRNA; in other words a Cpf1 system requires only the Cpf1 nuclease and a crRNA to cleave the target DNA sequence. Cpf1 endonucleases, are associated with T-rich PAM sites, e.g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the present invention and the choice of Cas protein will depend upon the particular conditions of the method.

Specific examples of Cas proteins include class II systems including Cas1, Cas2, Cas3, Cas4, Cas5, Cas6, Cas7, Cas8, Cas9, CaslO, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, the site-specific targeting moiety includes a sequence targeting polypeptide, such as an enzyme, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Crptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Cas protein is modified to deactivate the nuclease, e.g., nuclease-deficient Cas9, and to recruit transcription repressors, e.g., KRAB, or SID4X, to induce epigenetic modifications, e.g., histone methyltransferase and demethylase, histone deacetylase, DNA methyltransferase.

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are required by Cas9 for DNA cleavage to occur; for Cpf1 at least about 16 nucleotides of gRNA sequence is needed to achieve detectable DNA cleavage.

Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut the target DNA but interferes with transcription by steric hindrance. dCas9 can further be fused with a heterologous effector to repress (CRISPRi) a target gene. For example, Cas9 can be fused to a transcriptional silencer (e.g., a KRAB domain). A catalytically inactive Cas9 (dCas9) fused to Fokl nuclease (“dCas9-FokI”) can be used to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, MA 02139; addgene.org/crispr). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, an effector comprises one or more components of a CRISPR system described hereinabove.

In some embodiments, suitable effectors for use in the agents, compositions, and methods of the invention include, for example, nucleases, physical blockers, epigenetic recruiters, e.g., a transcriptional repressor and epigenetic CpG modifiers, e.g., a DNA methylase, a histone modifying agent, or a histone deacetylase, and combinations of any of the foregoing.

Suitable effectors include a polypeptide or its variant. The term “variant,” as used herein, refers to a polypeptide that is derived by incorporation of one or more amino acid insertions, substitutions, or deletions in a precursor polypeptide (e.g., “parent” polypeptide). In certain embodiments, a variant polypeptide has at least about 85% amino acid sequence identity, e.g., about 90%, about 91%, about 92%, about 93%, about 94%, about 95%, about 96%, about 97%, about 98%, about 99%, or about 100%, amino acid sequence identity to the entire amino acid sequence of a parent polypeptide.

The term “sequence identity,” as used herein, refers to a comparison between pairs of nucleic acid or amino acid molecules, i.e., the relatedness between two amino acid sequences or between two nucleotide sequences. In general, the sequences are aligned so that the highest order match is obtained. Methods for determining sequence identity are known and can be determined by commercially available computer programs that can calculate the percentage of identity between two or more sequences. A typical example of such a computer program is CLUSTAL.

Exemplary effectors include ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdbl), histone methyltransferase (SET2), euchromatic histone-lysine N-methyltransferase 2 (G9a), histone-lysine N-methyltransferase (SUV39H1), and G9a), histone deacetylase (e.g., HDACI, HDAC2, HDAC3), protein demethylases such as KDMIA and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, sybr green, and proflavine, efflux pump inhibitors such as peptidomimetics like phenylalanine arginyl-naphthylamide or quinoline derivatives, nuclear receptor activators and inhibitors, proteasome inhibitors, competitive inhibitors for enzymes such as those involved in lysosomal storage diseases, zinc finger proteins, TALENs, specific domains from proteins, such as a KRAB domain, an MeCP2 domain, an MQ1 domain, a DNMT3a-3L domain, protein synthesis inhibitors, nucleases (e.g., Cpf1, Cas9, zinc finger nuclease), fusions of one or more thereof (e.g., dCas9-DNMT, dCas9-APOBEC, dCas9-UGI,, dCas9-KRAB, dCas9-KRAB-MeCP2, dCas9-MQ1, dCas9-DNMT3a-3L, dCas9-EZH2, dCas9-HDAC8, dCas9-G9A, and dCas9-MC/MN).

In some embodiments, a suitable nuclease for use in the agent, compositions, and methods of the invention comprises a Cas9 polypeptide, or enzymatically active portion thereof. In one embodiment, the Cas9 polypeptide, or enzymatically active portion thereof, further comprises a catalytically active domain of human exonuclease 1 (hEXO1), e.g., 5′ to 3′ exonuclease activity and/or an RNase H activity. In other embodiments, a suitable nuclease comprises a transcription activator like effector nuclease (TALEN). In yet other embodiments, a suitable nuclease comprises a zinc finger protein.

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety.

TAL effectors (TALE) are proteins secreted by Xanthomonas bacteria. The DNA binding domain contains a highly conserved 33-34 amino acid sequence with the exception of the 12th and 13th amino acids. These two locations are highly variable (Repeat Variable Diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of the FokI endonuclease can be used to construct hybrid nucleases that are active in a yeast assay. These reagents are also active in plant cells and in animal cells. Initial TALEN studies used the wild-type FokI cleavage domain, but some subsequent TALEN studies also used FokI cleavage domain variants with mutations designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. The number of amino acid residues between the TALEN DNA binding domain and the FokI cleavage domain may be modified by introduction of a spacer (distinct from the spacer sequence) between the plurality of TAL effector repeat sequences and the FokI endonuclease domain. The spacer sequence may be 12 to 30 nucleotides, e.g., 12-15, 12-20, 20-25, or 15-30 nucleotides.

The relationship between amino acid sequence and DNA recognition of the TALE binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms. In this manner, they can be used to correct mutations in the genome which, for example, cause disease.

As used herein, a “zinc finger polypeptide” or “zinc finger protein” is a protein that binds to DNA, RNA and/or protein, in a sequence-specific manner, by virtue of a metal stabilized domain known as a zinc finger. Zinc finger proteins are nucleases having a DNA cleavage domain and a DNA binding zinc finger domain. Zinc finger polypeptides may be made by fusing the nonspecific DNA. cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

Zinc finger nucleases are chimeric enzymes made by fusing the nonspecific DNA. cleavage domain of the endonuclease FokI with site-specific DNA binding zinc finger domains. Due to the flexible nature of zinc finger proteins (ZFPs), ZFNs can be assembled that induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

In some embodiments, a suitable physical blocker for use in the agent, compositions, and methods of the invention comprises a gRNA, antisense DNA, or triplex forming oligonucleotide (which may target an expression control unit) steric block a transcriptional control element or anchoring sequence. The gRNA recognizes specific DNA sequences and further includes sequences that interfere with, e.g., a conjunction nucleating molecule sequence to act as a steric blocker. In some embodiments, the gRNA is combined with one or more peptides, e.g., S-adenosyl methionine (SAM), that acts as a steric presence. In other embodiments, a physical blocker comprises an enzymatically inactive Cas9 polypeptide, or fragment thereof (e.g., dCas9). In one embodiment, an epigenetic recruiter silences or represses transcription of a target gene. In some embodiments, a suitable epigenetic recruiter for use in the agent, compositions, and methods of the invention comprises a KRAB domain, or an MeCP2 domain.

In one embodiment, a suitable epigenetic recruiter for use in the agent, compositions, and methods of the invention comprises dCas9-MQ1 fusion, a dCas9-G9A fusion, a dCas9-EZH2 fusion, a dCas9-HDAC8 fusion, a dCas9-KRAB fusion, or a dCas9-KRAB-MeCP2 fusion.

As used herein, “KRAB” refers to a Krüppel associated box (KRAB) transcriptional repression domain present in human zinc finger protein-based transcription factors (KRAB zinc finger proteins).

As used herein, “MeCp2” refers to methyl CpG binding protein 2 which represses transcription, e.g., by binding to a promoter comprising methylated DNA.

In one embodiment, an epigenetic CpG modifier methylates DNA and inactivates or represses transcription. In some embodiments, a suitable epigenetic CpG modifier for use in the agent, compositions, and methods of the invention comprises a MQ1 domain or a DNMT3a-3L domain.

As used herein “MQ1” refers to a prokaryotic DNA methyltransferase.

As used herein “DNMT3a-3L” refers to a fusion of a DNA methyltransferase, Dnmt3a and a Dnmt3L which is catalytically inactive, but directly interacts with the catalytic domains of Dnmt3a.

In some embodiments, a suitable epigenetic recruiter for use in the agent, compositions, and methods of the invention comprises a MQ1 domain or a DNMT3a-3L domain. In one embodiment, a suitable epigenetic recruiter for use in the agent, compositions, and methods of the invention comprises a dCas9-MQ1 fusion or a dCas9-DNMT3a-3L fusion.

TABLE 11
Effector Polypeptide
HDAC8 (NP_060956.1)

| | |
|---|---|
| HDAC8 | GAAGAACCTGAGGAGCCTGCCGATTCCGGACAGAGCCTGGTGCC |
| (NP_060956.1) | CGTGTATATCTACTCCCCCGAATACGTGTCTATGTGCGACAGCTT |
| Effector DNA | GGCTAAGATTCCGAAGCGGGCATCAATGGTGCACTCGCTGATCGA |
| sequence | GGCCTACGCCCTGCACAAGCAGATGAGAATCGTGAAGCCCAAGG |
| (SEQ ID NO: 72) | TCGCCTCAATGGAAGAGATGGCCACCTTCCACACCGACGCCTACT |
| | TGCAACATCTCCAAAAAGTGTCCCAGGAAGGGGACGACGACCAC |
| | CCTGACTCCATTGAGTACGGCCTGGGTTACGACTGCCCAGCTACC |
| | GAAGGGATCTTCGACTACGCCGCTGCCATTGGCGGAGCGACCATC |
| | ACTGCCGCGCAGTGCCTCATCGACGGAATGTGCAAGGTCGCCATC |
| | AACTGGTCCGGAGGCTGGCACCACGCCAAGAAGGATGAAGCCTC |
| | CGGCTTCTGCTACCTTAACGACGCCGTGCTGGGAATCCTTCGCCT |
| | GCGAAGAAAGTTCGAACGGATTTTGTATGTGGACCTGGATCTGCA |
| | TCATGGAGATGGCGTGGAGGATGCCTTCTCCTTCACCTCGAAAGT |
| | CATGACCGTGTCACTTCACAAGTTCTCACCCGGCTTCTTCCCTGGG |
| | ACCGGAGATGTGTCCGACGTCGGTCTGGGAAAGGGCCGCTACTA |
| | CTCCGTGAACGTGCCGATCCAGGACGGTATCCAGGACGAGAAGT |
| | ACTACCAGATCTGCGAGAGCGTGCTGAAGGAAGTGTACCAGGCC |
| | TTCAATCCCAAGGCCGTGGTGCTGCAGCTTGGCGCGGACACTATT |
| | GCGGGCGACCCGATGTGCTCCTTCAACATGACCCCCGTCGGTATT |
| | GGAAAGTGCCTGAAGTACATCCTGCAATGGCAGCTGGCTACCCTG |
| | ATCCTGGGAGGAGGCGGATACAACCTCGCAAACACCGCCCGGTG |
| | TTGGACTTACCTGACTGGGGTCATCCTGGGAAAGACTCTGTCATC |
| | CGAGATTCCCGACCACGAGTTCTTCACCGCGTACGGCCCAGATTA |
| | CGTGCTCGAAATCACTCCTTCCTGCCGGCCTGATAGAAATGAGCC |
| | GCACCGCATACAGCAGATCCTCAACTACATTAAGGGCAACCTGA |
| | AGCACGTGGTG |
| HDAC8 | EEPEEPADSGQSLVPVYIYSPEYVSMCDSLAKIPKRASMVHSLIEAYA |
| (NP_060956.1) | LHKQMRIVKPKVASMEEMATFHTDAYLQHLQKVSQEGDDDHPDSIE |
| Effector Protein | YGLGYDCPATEGIFDYAAAIGGATITAAQCLIDGMCKVAINWSGGW |
| Sequence | HHAKKDEASGFCYLNDAVLGILRLRRKFERILYVDLDLHHGDGVED |
| (SEQ ID NO: 76) | AFSFTSKVMTVSLHKFSPGFFPGTGDVSDVGLGKGRYYSVNVPIQDG |
| | IQDEKYYQICESVLKEVYQAFNPKAVVLQLGADTIAGDPMCSFNMT |
| | PVGIGKCLKYILQWQLATLILGGGGYNLANTARCWTYLTGVILGKT |
| | LSSEIPDHEFFTAYGPDYVLEITPSCRPDRNEPHRIQQILNYIKGNLKH |
| | VV |
| HDAC8 | GAAGAACCUGAGGAGCCUGCCGAUUCCGGACAGAGCCUGGUGC |
| (NP_060956.1) | CCGUGUAUAUCUACUCCCCCGAAUACGUGUCUAUGUGCGACAG |
| Effector RNA | CUUGGCUAAGAUUCCGAAGCGGGCAUCAAUGGUGCACUCGCUG |
| sequence | AUCGAGGCCUACGCCCUGCACAAGCAGAUGAGAAUCGUGAAGC |
| (SEQ ID NO: 80) | CCAAGGUCGCCUCAAUGGAAGAGAUGGCCACCUUCCACACCGAC |
| | GCCUACUUGCAACAUCUCCAAAAAGUGUCCCAGGAAGGGGACG |
| | ACGACCACCCUGACUCCAUUGAGUACGGCCUGGGUUACGACUG |
| | CCCAGCUACCGAAGGGAUCUUCGACUACGCCGCUGCCAUUGGCG |
| | GAGCGACCAUCACUGCCGCGCAGUGCCUCAUCGACGGAAUGUG |
| | CAAGGUCGCCAUCAACUGGUCCGGAGGCUGGCACCACGCCAAG |
| | AAGGAUGAAGCCUCCGGCUUCUGCUACCUUAACGACGCCGUGC |
| | UGGGAAUCCUUCGCCUGCGAAGAAAGUUCGAACGGAUUUUGUA |
| | UGUGGACCUGGAUCUGCAUCAUGGAGAUGGCGUGGAGGAUGCC |
| | UUCUCCUUCACCUCGAAAGUCAUGACCGUGUCACUUCACAAGU |
| | UCUCACCCGGCUUCUUCCCUGGGACCGGAGAUGUGUCCGACGUC |
| | GGUCUGGGAAAGGGCCGCUACUACUCCGUGAACGUGCCGAUCC |
| | AGGACGGUAUCCAGGACGAGAAGUACUACCAGAUCUGCGAGAG |
| | CGUGCUGAAGGAAGUGUACCAGGCCUUCAAUCCCAAGGCCGUG |
| | GUGCUGCAGCUUGGCGCGGACACUAUUGCGGGCGACCCGAUGU |
| | GCUCCUUCAACAUGACCCCCGUCGGUAUUGGAAAGUGCCUGAA |
| | GUACAUCCUGCAAUGGCAGCUGGCUACCCUGAUCCUGGGAGGA |
| | GGCGGAUACAACCUCGCAAACACCGCCCGGUGUUGGACUUACC |
| | UGACUGGGGUCAUCCUGGGAAAGACUCUGUCAUCCGAGAUUCC |
| | CGACCACGAGUUCUUCACCGCGUACGGCCCAGAUUACGUGCUCG |
| | AAAUCACUCCUUCCUGCCGGCCUGAUAGAAAUGAGCCGCACCGC |
| | AUACAGCAGAUCCUCAACUACAUUAAGGGCAACCUGAAGCACG |
| | UGGUG |

G9A (NP_006700.3)

| | |
|---|---|
| G9A (NP_006700.3) | GGAAGCGCCGCTATCGCCGAAGTGCTGCTGAATGCCAGATGCGA |
| Effector DNA | TCTGCATGCCGTGAACTACCACGGCGACACCCCTCTGCATATCGC |
| sequence | CGCTAGAGAGAGCTACCATGACTGTGTGCTGCTGTTTCTGAGCAG |
| (SEQ ID NO: 73) | AGGCGCCAATCCCGAGCTCAGAAACAAAGAGGGCGACACCGCTT |
| | GGGATCTGACACCCGAGAGATCCGACGTGTGGTTCGCTCTGCAAC |
| | TGAATAGAAAACTGAGACTGGGCGTCGGCAATAGGGCCATTAGA |
| | ACCGAGAAGATCATCTGTAGGGACGTGGCTAGGGGCTACGAGAA |
| | CGTGCCCATCCCTTGTGTGAACGGAGTGGATGGAGAGCCTTGCCC |
| | CGAGGATTACAAATACATCAGCGAGAACTGCGAAACCTCCACCA |
| | TGAATATCGATAGAAACATTACACACCTCCAGCACTGTACATGCG |
| | TGGACGATTGCAGCAGCAGCAACTGTCTGTGCGGCCAACTGAGC |
| | ATCAGATGCTGGTACGACAAGGATGGCAGACTGCTGCAAGAGTT |
| | CAACAAGATCGAACCCCCTCTGATCTTCGAGTGTAACCAAGCTTG |
| | CAGCTGTTGGAGGAACTGCAAGAATAGGGTCGTGCAGTCCGGAA |
| | TCAAGGTGAGACTGCAGCTGTATAGAACAGCTAAGATGGGATGG |
| | GGAGTCAGAGCTCTGCAGACCATCCCCCAAGGCACATTCATCTGT |
| | GAGTACGTCGGCGAACTCATCAGCGACGCTGAGGCCGATGTGAG |
| | GGAGGACGACAGCTATCTCTTCGACCTCGACAACAAGGACGGCG |
| | AGGTGTACTGCATCGACGCTAGATATTACGGCAACATCAGCAGAT |
| | TCATCAACCACCTCTGCGACCCCAATATCATCCCCGTGAGAGTGT |
| | TCATGCTCCATCAAGATCTGAGATTCCCTAGGATCGCCTTCTTCAG |
| | CTCTAGAGACATTAGAACCGGCGAGGAGCTGGGATTCGACTACG |
| | GCGACAGGTTCTGGGACATCAAGAGCAAGTACTTCACATGCCAAT |
| | GCGGCAGCGAGAAATGCAAGCATAGCGCCGAGGCCATTGCTCTG |
| | GAGCAGTCTAGACTGGCTAGGCTGGACCCTCACCCCGAGCTGCTG |
| | CCCGAACTGGGATCTCTGCCTCCCGTGAATACC |
| G9A (NP_006700.3) | GSAAIAEVLLNARCDLHAVNYHGDTPLHIAARESYHDCVLLFLSRG |
| Effector Protein | ANPELRNKEGDTAWDLTPERSDVWFALQLNRKLRLGVGNRAIRTEK |
| Sequence | IICRDVARGYENVPIPCVNGVDGEPCPEDYKYISENCETSTMNIDRNI |
| (SEQ ID NO: 77) | THLQHCTCVDDCSSSNCLCGQLSIRCWYDKDGRLLQEFNKIEPPLIFE |
| | CNQACSCWRNCKNRVVQSGIKVRLQLYRTAKMGWGVRALQTIPQG |
| | TFICEYVGELISDAEADVREDDSYLFDLDNKDGEVYCIDARYYGNIS |
| | RFINHLCDPNIIPVRVFMLHQDLRFPRIAFFSSRDIRTGEELGFDYGDR |
| | FWDIKSKYFTCQCGSEKCKHSAEAIALEQSRLARLDPHPELLPELGSL |
| | PPVNT |
| G9A (NP_006700.3) | GGAAGCGCCGCUAUCGCCGAAGUGCUGCUGAAUGCCAGAUGCG |
| Effector RNA | AUCUGCAUGCCGUGAACUACCACGGCGACACCCCUCUGCAUAUC |
| sequence | GCCGCUAGAGAGAGCUACCAUGACUGUGUGCUGCUGUUUCUGA |
| (SEQ ID NO: 81) | GCAGAGGCGCCAAUCCCGAGCUCAGAAACAAAGAGGGCGACAC |
| | CGCUUGGGAUCUGACACCCGAGAGAUCCGACGUGUGGUUCGCU |
| | CUGCAACUGAAUAGAAAACUGAGACUGGGCGUCGGCAAUAGGG |
| | CCAUUAGAACCGAGAAGAUCAUCUGUAGGGACGUGGCUAGGGG |
| | CUACGAGAACGUGCCCAUCCCUUGUGUGAACGGAGUGGAUGGA |
| | GAGCCUUGCCCCGAGGAUUACAAAUACAUCAGCGAGAACUGCG |
| | AAACCUCCACCAUGAAUAUCGAUAGAAACAUUACACACCUCCA |
| | GCACUGUACAUGCGUGGACGAUUGCAGCAGCAGCAACUGUCUG |
| | UGCGGCCAACUGAGCAUCAGAUGCUGGUACGACAAGGAUGGCA |
| | GACUGCUGCAAGAGUUCAACAAGAUCGAACCCCCUCUGAUCUU |
| | CGAGUGUAACCAAGCUUGCAGCUGUUGGAGGAACUGCAAGAAU |
| | AGGGUCGUGCAGUCCGGAAUCAAGGUGAGACUGCAGCUGUAUA |
| | GAACAGCUAAGAUGGGAUGGGGAGUCAGAGCUCUGCAGACCAU |
| | CCCCCAAGGCACAUUCAUCUGUGAGUACGUCGGCGAACUCAUC |
| | AGCGACGCUGAGGCCGAUGUGAGGGAGGACGACAGCUAUCUCU |
| | UCGACCUCGACAACAAGGACGGCGAGGUGUACUGCAUCGACGC |
| | UAGAUAUUACGGCAACAUCAGCAGAUUCAUCAACCACCUCUGC |
| | GACCCCAAUAUCAUCCCCGUGAGAGUGUUCAUGCUCCAUCAAG |
| | AUCUGAGAUUCCCUAGGAUCGCCUUCUUCAGCUCUAGAGACAU |
| | UAGAACCGGCGAGGAGCUGGGAUUCGACUACGGCGACAGGUUC |
| | UGGGACAUCAAGAGCAAGUACUUCACAUGCCAAUGCGGCAGCG |
| | AGAAAUGCAAGCAUAGCGCCGAGGCCAUUGCUCUGGAGCAGUC |
| | UAGACUGGCUAGGCUGGACCCUCACCCCGAGCUGCUGCCCGAAC |
| | UGGGAUCUCUGCCUCCCGUGAAUACC |

EZH2 (NP_001190176.1)

| | |
|---|---|
| EZH2 | GGACAGACGGGCAAAAAGAGCGAAAAGGGACCCGTCTGCTGGCG |
| (NP_001190176.1) | GAAACGAGTGAAAAGCGAATACATGCGCCTGCGGCAACTCAAGA |
| Effector DNA | GGTTCCGCAGAGCCGACGAGGTCAAGAGCATGTTCAGCAGCAAC |
| sequence | AGACAAAAGATCCTGGAGAGGACCGAAATCCTCAACCAGGAATG |
| (SEQ ID NO: 74) | GAAGCAGCGCAGGATCCAGCCGGTGCACATCCTGACCAGCGTCA |
| | GCAGCCTCAGAGGCACCCGCGAGTGCAGCGTGACCAGCGACCTG |
| | GACTTCCCCACCCAAGTGATCCCGCTGAAAACCCTCAACGCAGTG |
| | GCCAGCGTGCCGATCATGTACAGCTGGAGCCCCCTGCAGCAGAA |
| | CTTCATGGTCGAGGACGAAACCGTGCTGCACAACATCCCGTACAT |
| | GGGCGACGAAGTGCTGGACCAGGACGGAACCTTCATCGAGGAAC |
| | TGATCAAGAACTACGACGGAAAGGTGCACGGGGACAGGGAATGC |
| | GGGTTCATCAACGACGAGATCTTCGTCGAGCTCGTCAACGCCCTG |
| | GGACAATACAACGACGACGACGACGACGACGACGGGGACGACCC |
| | AGAAGAACGGGAGGAAAAGCAGAAGGACCTGGAGGACCACCGC |
| | GACGACAAGGAAAGCAGACCCCCCAGGAAGTTCCCGAGCGACAA |
| | GATCTTCGAAGCCATCAGCAGCATGTTCCCGGACAAGGGAACCG |
| | CCGAGGAGCTGAAGGAAAAGTACAAGGAACTGACCGAGCAGCA |
| | GCTCCCAGGAGCACTGCCGCCGGAATGCACACCAAACATCGACG |
| | GACCGAACGCCAAGAGCGTGCAGAGGGAGCAGAGCCTGCACAGC |
| | TTCCACACCCTGTTCTGCCGGAGATGCTTCAAATACGACTGCTTCC |
| | TGCACCCCTTCCACGCCACCCCGAACACCTACAAGCGCAAGAACA |
| | CCGAAACCGCCCTGGACAACAAGCCGTGCGGACCCCAATGCTAC |
| | CAGCACCTGGAAGGCGCCAAGGAGTTCGCCGCAGCACTGACCGC |
| | CGAGCGGATCAAAACCCCGCCGAAAAGACCCGGAGGCAGACGGA |
| | GAGGGAGACTCCCAAACAACAGCAGCAGACCGAGCACGCCGACC |
| | ATCAACGTGCTCGAGAGCAAGGACACCGACAGCGACAGGGAAGC |
| | GGGAACCGAAACCGGAGGAGAAAACAACGACAAGGAGGAGGAA |
| | GAAAAGAAGGACGAGACAAGCAGCAGCAGCGAAGCCAACAGCA |
| | GATGCCAAACCCCGATCAAGATGAAGCCGAACATCGAGCCGCCG |
| | GAGAACGTGGAATGGAGCGGAGCCGAAGCGAGCATGTTCCGGGT |
| | GCTGATCGGCACCTACTACGACAACTTCTGCGCCATCGCACGCCT |
| | GATCGGAACCAAGACGTGCCGCCAGGTCTACGAGTTCCGCGTCA |
| | AGGAGAGCAGCATCATCGCACCAGCACCAGCCGAGGACGTCGAC |
| | ACCCCACCACGGAAGAAGAAGCGGAAACACAGACTCTGGGCCGC |
| | ACACTGCAGAAAGATCCAGCTGAAGAAGGACGGAAGCAGCAACC |
| | ACGTGTACAACTACCAACCATGCGACCACCCGAGACAACCCTGC |
| | GACAGCAGCTGCCCCTGCGTGATCGCCCAGAACTTCTGCGAAAAG |
| | TTCTGCCAGTGCAGCAGCGAGTGCCAGAACAGGTTCCCAGGATGC |
| | CGCTGCAAGGCCCAGTGCAACACCAAGCAATGCCCATGCTACCTC |
| | GCCGTGAGGGAGTGCGACCCGGACCTGTGCCTCACATGCGGAGC |
| | AGCGGACCACTGGGACAGCAAGAACGTCAGCTGCAAGAACTGCA |
| | GCATCCAGAGAGGAAGCAAGAAGCACCTCCTGCTGGCGCCAAGC |
| | GACGTGGCCGGATGGGGAATCTTCATCAAGGACCCAGTCCAAAA |
| | GAACGAATTCATCAGCGAATACTGCGGAGAAATCATCAGCCAGG |
| | ACGAAGCCGACCGCAGAGGGAAGGTGTACGACAAGTACATGTGC |
| | AGCTTCCTCTTCAACCTGAACAACGACTTCGTAGTGGACGCCACC |
| | AGAAAGGGCAACAAGATCCGCTTCGCCAACCACAGCGTGAACCC |
| | GAACTGCTACGCGAAGGTCATGATGGTGAACGGCGACCACCGGA |
| | TCGGGATCTTCGCCAAGCGGGCCATCCAAACCGGAGAGGAGCTG |
| | TTCTTCGACTACCGGTACAGCCAAGCGGACGCCCTGAAATACGTG |
| | GGCATCGAGCGGGAGATGGAGATCCCA |
| EZH2 | GQTGKKSEKGPVCWRKRVKSEYMRLRQLKRFRRADEVKSMFSSNR |
| (NP_001190176.1) | QKILERTEILNQEWKQRRIQPVHILTSVSSLRGTRECSVTSDLDFPTQV |
| Effector Protein | IPLKTLNAVASVPIMYSWSPLQQNFMVEDETVLHNIPYMGDEVLDQ |
| Sequence | DGTFIEELIKNYDGKVHGDRECGFINDEIFVELVNALGQYNDDDDDD |
| (SEQ ID NO: 78) | DGDDPEEREEKQKDLEDHRDDKESRPPRKFPSDKIFEAISSMFPDKGT |
| | AEELKEKYKELTEQQLPGALPPECTPNIDGPNAKSVQREQSLHSFHTL |
| | FCRRCFKYDCFLHPFHATPNTYKRKNTETALDNKPCGPQCYQHLEG |
| | AKEFAAALTAERIKTPPKRPGGRRRGRLPNNSSRPSTPTINVLESKDT |
| | DSDREAGTETGGENNDKEEEEKKDETSSSSEANSRCQTPIKMKPNIEP |
| | PENVEWSGAEASMFRVLIGTYYDNFCAIARLIGTKTCRQVYEFRVKE |
| | SSIIAPAPAEDVDTPPRKKKRKHRLWAAHCRKIQLKKDGSSNHVYN |
| | YQPCDHPRQPCDSSCPCVIAQNFCEKFCQCSSECQNRFPGCRCKAQC |
| | NTKQCPCYLAVRECDPDLCLTCGAADHWDSKNVSCKNCSIQRGSKK |
| | HLLLAPSDVAGWGIFIKDPVQKNEFISEYCGEIISQDEADRRGKVYDK |
| | YMCSFLFNLNNDFVVDATRKGNKIRFANHSVNPNCYAKVMMVNGD |
| | HRIGIFAKRAIQTGEELFFDYRYSQADALKYVGIEREMEIP |
| EZH2 | GGACAGACGGGCAAAAAGAGCGAAAAGGGACCCGUCUGCUGGC |
| (NP_001190176.1) | GGAAACGAGUGAAAAGCGAAUACAUGCGCCUGCGGCAACUCAA |
| Effector RNA | GAGGUUCCGCAGAGCCGACGAGGUCAAGAGCAUGUUCAGCAGC |
| sequence | AACAGACAAAAGAUCCUGGAGAGGACCGAAAUCCUCAACCAGG |
| (SEQ ID NO: 82) | AAUGGAAGCAGCGCAGGAUCCAGCCGGUGCACAUCCUGACCAG |
| | CGUCAGCAGCCUCAGAGGCACCCGCGAGUGCAGCGUGACCAGCG |
| | ACCUGGACUUCCCCACCCAAGUGAUCCCGCUGAAAACCCUCAAC |
| | GCAGUGGCCAGCGUGCCGAUCAUGUACAGCUGGAGCCCCCUGC |
| | AGCAGAACUUCAUGGUCGAGGACGAAACCGUGCUGCACAACAU |
| | CCCGUACAUGGGCGACGAAGUGCUGGACCAGGACGGAACCUUC |
| | AUCGAGGAACUGAUCAAGAACUACGACGGAAAGGUGCACGGGG |
| | ACAGGGAAUGCGGGUUCAUCAACGACGAGAUCUUCGUCGAGCU |
| | CGUCAACGCCCUGGGACAAUACAACGACGACGACGACGACGAC |
| | GACGGGGACGACCCAGAAGAACGGGAGGAAAAGCAGAAGGACC |
| | UGGAGGACCACCGCGACGACAAGGAAAGCAGACCCCCCAGGAA |
| | GUUCCCGAGCGACAAGAUCUUCGAAGCCAUCAGCAGCAUGUUC |
| | CCGGACAAGGGAACCGCCGAGGAGCUGAAGGAAAAGUACAAGG |
| | AACUGACCGAGCAGCAGCUCCCAGGAGCACUGCCGCCGGAAUGC |
| | ACACCAAACAUCGACGGACCGAACGCCAAGAGCGUGCAGAGGG |
| | AGCAGAGCCUGCACAGCUUCCACACCCUGUUCUGCCGGAGAUGC |
| | UUCAAAUACGACUGCUUCCUGCACCCCUUCCACGCCACCCCGAA |
| | CACCUACAAGCGCAAGAACACCGAAACCGCCCUGGACAACAAGC |
| | CGUGCGGACCCCAAUGCUACCAGCACCUGGAAGGCGCCAAGGA |
| | GUUCGCCGCAGCACUGACCGCCGAGCGGAUCAAAACCCCGCCGA |
| | AAAGACCCGGAGGCAGACGGAGAGGGAGACUCCCAAACAACAG |
| | CAGCAGACCGAGCACGCCGACCAUCAACGUGCUCGAGAGCAAG |
| | GACACCGACAGCGACAGGGAAGCGGGAACCGAAACCGGAGGAG |
| | AAAACAACGACAAGGAGGAGGAAGAAAAGAAGGACGAGACAAG |
| | CAGCAGCAGCGAAGCCAACAGCAGAUGCCAAACCCCGAUCAAG |
| | AUGAAGCCGAACAUCGAGCCGCCGGAGAACGUGGAAUGGAGCG |
| | GAGCCGAAGCGAGCAUGUUCCGGGUGCUGAUCGGCACCUACUA |
| | CGACAACUUCUGCGCCAUCGCACGCCUGAUCGGAACCAAGACGU |
| | GCCGCCAGGUCUACGAGUUCCGCGUCAAGGAGAGCAGCAUCAU |
| | CGCACCAGCACCAGCCGAGGACGUCGACACCCCACCACGGAAGA |
| | AGAAGCGGAAACACAGACUCUGGGCCGCACACUGCAGAAAGAU |
| | CCAGCUGAAGAAGGACGGAAGCAGCAACCACGUGUACAACUAC |
| | CAACCAUGCGACCACCCGAGACAACCCUGCGACAGCAGCUGCCC |
| | CUGCGUGAUCGCCCAGAACUUCUGCGAAAAGUUCUGCCAGUGC |
| | AGCAGCGAGUGCCAGAACAGGUUCCCAGGAUGCCGCUGCAAGG |
| | CCCAGUGCAACACCAAGCAAUGCCCAUGCUACCUCGCCGUGAGG |
| | GAGUGCGACCCGGACCUGUGCCUCACAUGCGGAGCAGCGGACC |
| | ACUGGGACAGCAAGAACGUCAGCUGCAAGAACUGCAGCAUCCA |
| | GAGAGGAAGCAAGAAGCACCUCCUGCUGGCGCCAAGCGACGUG |
| | GCCGGAUGGGGAAUCUUCAUCAAGGACCCAGUCCAAAAGAACG |
| | AAUUCAUCAGCGAAUACUGCGGAGAAAUCAUCAGCCAGGACGA |
| | AGCCGACCGCAGAGGGAAGGUGUACGACAAGUACAUGUGCAGC |
| | UUCCUCUUCAACCUGAACAACGACUUCGUAGUGGACGCCACCA |
| | GAAAGGGCAACAAGAUCCGCUUCGCCAACCACAGCGUGAACCC |
| | GAACUGCUACGCGAAGGUCAUGAUGGUGAACGGCGACCACCGG |
| | AUCGGGAUCUUCGCCAAGCGGGCCAUCCAAACCGGAGAGGAGC |
| | UGUUCUUCGACUACCGGUACAGCCAAGCGGACGCCCUGAAAUA |
| | CGUGGGCAUCGAGCGGGAGAUGGAGAUCCCA |

MQ1 (P15840.3)

| | |
|---|---|
| MQ1 (P15840.3) | AGCAAGGTGGAGAACAAGACCAAGAAGCTGCGGGTGTTCGAGGC |
| Effector DNA | CTTCGCCGGCATCGGCGCCCAGCGGAAGGCCCTGGAGAAGGTGC |
| sequence | GGAAGGACGAGTACGAGATCGTGGGCCTGGCCGAGTGGTACGTG |
| (SEQ ID NO: 75) | CCCGCCATCGTGATGTACCAGGCCATCCACAACAACTTCCACACC |
| | AAGCTGGAGTACAAGAGCGTGAGCCGGGAGGAGATGATCGACTA |
| | CCTGGAGAACAAGACCCTGAGCTGGAACAGCAAGAACCCCGTGA |
| | GCAACGGCTACTGGAAGCGGAAGAAGGACGACGAGCTGAAGATC |
| | ATCTACAACGCCATCAAGCTGAGCGAGAAGGAGGGCAACATCTT |
| | CGACATCCGGGACCTGTACAAGCGGACCCTGAAGAACATCGACC |
| | TGCTGACCTACAGCTTCCCCTGCCAGGACCTGAGCCAGCAGGGCA |
| | TCCAGAAGGGCATGAAGCGGGGCAGCGGCACCCGGAGCGGCCTG |
| | CTGTGGGAGATCGAGCGGGCCCTGGACAGCACCGAGAAGAACGA |
| | CCTGCCCAAGTACCTGCTGATGGAGAACGTGGGCGCCCTGCTGCA |
| | CAAGAAGAACGAGGAGGAGCTGAACCAGTGGAAGCAGAAGCTG |
| | GAGAGCCTGGGCTACCAGAACAGCATCGAGGTGCTGAACGCCGC |
| | CGACTTCGGCAGCAGCCAGGCCCGGCGGCGGGTGTTCATGATCA |
| | GCACCCTGAACGAGTTCGTGGAGCTGCCCAAGGGCGACAAGAAG |
| | CCCAAGAGCATCAAGAAGGTGCTGAACAAGATCGTGAGCGAGAA |
| | GGACATCCTGAACAACCTGCTGAAGTACAACCTGACCGAGTTCAA |
| | GAAAACCAAGAGCAACATCAACAAGGCCAGCCTGATCGGCTACA |
| | GCAAGTTCAACAGCGAGGGCTACGTGTACGACCCCGAGTTCACC |
| | GGCCCCACCCTGACCGCCAGCGGCGCCAACAGCCGGATCAAGAT |
| | CAAGGACGGCAGCAACATCCGGAAGATGAACAGCGACGAGACCT |
| | TCCTGTACATCGGCTTCGACAGCCAGGACGGCAAGCGGGTGAAC |
| | GAGATCGAGTTCCTGACCGAGAACCAGAAGATCTTCGTGTGCGGC |
| | AACAGCATCAGCGTGGAGGTGCTGGAGGCCATCATCGACAAGAT |
| | CGGCGGC |
| MQ1 (P15840.3) | SKVENKTKKLRVFEAFAGIGAQRKALEKVRKDEYEIVGLAEWYVPA |
| Effector Protein | IVMYQAIHNNFHTKLEYKSVSREEMIDYLENKTLSWNSKNPVSNGY |
| Sequence | WKRKKDDELKIIYNAIKLSEKEGNIFDIRDLYKRTLKNIDLLTYSFPC |
| (SEQ ID NO: 79) | QDLSQQGIQKGMKRGSGTRSGLLWEIERALDSTEKNDLPKYLLMEN |
| | VGALLHKKNEEELNQWKQKLESLGYQNSIEVLNAADFGSSQARRRV |
| | FMISTLNEFVELPKGDKKPKSIKKVLNKIVSEKDILNNLLKYNLTEFK |
| | KTKSNINKASLIGYSKFNSEGYVYDPEFTGPTLTASGANSRIKIKDGS |
| | NIRKMNSDETFLYIGFDSQDGKRVNEIEFLTENQKIFVCGNSISVEVLE |
| | AIIDKIGG |
| MQ1 (P15840.3) | AGCAAGGUGGAGAACAAGACCAAGAAGCUGCGGGUGUUCGAGG |
| Effector RNA | CCUUCGCCGGCAUCGGCGCCCAGCGGAAGGCCCUGGAGAAGGU |
| sequence | GCGGAAGGACGAGUACGAGAUCGUGGGCCUGGCCGAGUGGUAC |
| (SEQ ID NO: 83) | GUGCCCGCCAUCGUGAUGUACCAGGCCAUCCACAACAACUUCCA |
| | CACCAAGCUGGAGUACAAGAGCGUGAGCCGGGAGGAGAUGAUC |
| | GACUACCUGGAGAACAAGACCCUGAGCUGGAACAGCAAGAACC |
| | CCGUGAGCAACGGCUACUGGAAGCGGAAGAAGGACGACGAGCU |
| | GAAGAUCAUCUACAACGCCAUCAAGCUGAGCGAGAAGGAGGGC |
| | AACAUCUUCGACAUCCGGGACCUGUACAAGCGGACCCUGAAGA |
| | ACAUCGACCUGCUGACCUACAGCUUCCCCUGCCAGGACCUGAGC |
| | CAGCAGGGCAUCCAGAAGGGCAUGAAGCGGGGCAGCGGCACCC |
| | GGAGCGGCCUGCUGUGGGAGAUCGAGCGGGCCCUGGACAGCAC |
| | CGAGAAGAACGACCUGCCCAAGUACCUGCUGAUGGAGAACGUG |
| | GGCGCCCUGCUGCACAAGAAGAACGAGGAGGAGCUGAACCAGU |
| | GGAAGCAGAAGCUGGAGAGCCUGGGCUACCAGAACAGCAUCGA |
| | GGUGCUGAACGCCGCCGACUUCGGCAGCAGCCAGGCCCGGCGGC |
| | GGGUGUUCAUGAUCAGCACCCUGAACGAGUUCGUGGAGCUGCC |
| | CAAGGGCGACAAGAAGCCCAAGAGCAUCAAGAAGGUGCUGAAC |
| | AAGAUCGUGAGCGAGAAGGACAUCCUGAACAACCUGCUGAAGU |
| | ACAACCUGACCGAGUUCAAGAAAACCAAGAGCAACAUCAACAA |
| | GGCCAGCCUGAUCGGCUACAGCAAGUUCAACAGCGAGGGCUAC |
| | GUGUACGACCCCGAGUUCACCGGCCCCACCCUGACCGCCAGCGG |
| | CGCCAACAGCCGGAUCAAGAUCAAGGACGGCAGCAACAUCCGG |
| | AAGAUGAACAGCGACGAGACCUUCCUGUACAUCGGCUUCGACA |
| | GCCAGGACGGCAAGCGGGUGAACGAGAUCGAGUUCCUGACCGA |
| | GAACCAGAAGAUCUUCGUGUGCGGCAACAGCAUCAGCGUGGAG |
| | GUGCUGGAGGCCAUCAUCGACAAGAUCGGCGGC |

KRAB

| | |
|---|---|
| KRAB | GACGCCAAGAGCCTGACCGCCTGGAGCCGGACCCTGGTGACCTTC |
| Effector DNA | AAGGACGTGTTCGTGGACTTCACCCGGGAGGAGTGGAAGCTGCT |
| sequence | GGACACCGCCCAGCAGATCCTGTACCGGAACGTGATGCTGGAGA |
| (SEQ ID NO: 302) | ACTACAAGAACCTGGTGAGCCTGGGCTACCAGCTGACCAAGCCC |
| | GACGTGATCCTGCGGCTGGAGAAGGGCGAGGAGCCCTGGCTGGT |
| | GGAGCGGGAGATCCACCAGGAAACCCACCCCGACAGCGAAACCG |
| | CCTTCGAGATCAAGAGCAGCGTG |
| KRAB | DAKSLTAWSRTLVTFKDVFVDFTREEWKLLDTAQQILYRNVMLENY |
| Effector Protein | KNLVSLGYQLTKPDVILRLEKGEEPWLVEREIHQETHPDSETAFEIKS |
| Sequence | SV |
| (SEQ ID NO: 297) | |
| KRAB | GACGCCAAGAGCCUGACCGCCUGGAGCCGGACCCUGGUGACCUU |
| Effector RNA | CAAGGACGUGUUCGUGGACUUCACCCGGGAGGAGUGGAAGCUG |
| sequence | CUGGACACCGCCCAGCAGAUCCUGUACCGGAACGUGAUGCUGG |
| (SEQ ID NO: 298) | AGAACUACAAGAACCUGGUGAGCCUGGGCUACCAGCUGACCAA |
| | GCCCGACGUGAUCCUGCGGCUGGAGAAGGGCGAGGAGCCCUGG |
| | CUGGUGGAGCGGGAGAUCCACCAGGAGACCCACCCCGACAGCG |
| | AGACCGCCUUCGAGAUCAAGAGCAGCGUG |

### Delivery of a Site-Specific Epigenetic Modifying Agent of the Invention and Compositions Comprising a Site-Specific an Epigenetic Modifying Agent of the Invention

The delivery of the epigenetic modifying agents of the invention to a cell e.g., a cell within a subject, such as a human subject (e.g., a subject in need thereof, such as a subject having a disease or disorder associated with CXCL9, CXCL10, and/or CXCL11, e.g., liver disease) may be achieved in a number of different ways. For example, delivery may be performed by contacting a cell with an epigenetic modifying agent of the invention either in vitro, ex vivo, or in vivo. In vivo delivery may be performed directly by administering a composition, such as a lipid composition, comprising an epigenetic modifying agent to a subject. Alternatively, in vivo delivery may be performed indirectly by administering one or more vectors that encode and direct the expression of the epigenetic modifying agent in a cell of a subject. These alternatives are discussed further below. In vitro introduction into a cell includes methods known in the art such as electroporation and lipofection. Further approaches are described herein below and/or are known in the art.

In some embodiments, the epigenetic modifying agent comprises a nucleic acid molecule encoding a fusion protein, the fusion protein comprising a site-specific targeting moiety, such as a polynucleotide encoding a DNA-binding domain of a Transcription activator-like effector (TALE) polypeptide or a zinc finger (ZFP) polypeptide, or fragment thereof, that specifically targets and binds to the expression control region associated with CXCL9, CXCL10, and/or CXCL11 and an effector molecule, such as a MQ1 or KRAB.

In other embodiments, the epigenetic modifying agent comprises a guide RNA and an mRNA encoding an effector molecule. The ratio of guide RNA to mRNA may be about 100:1 to about 1:100 (wt:wt).

In general, any method of delivery of a site-specific epigenetic modifying agent of the invention (in vitro, ex vivo, or in vivo) may be adapted for use with the epigenetic modifying agents of the invention (see e.g., Akhtar S. and Julian R L., (1992) Trends Cell. Biol. 2 (5): 139-144 and WO94/02595, which are incorporated herein by reference in their entireties). For in vivo delivery, factors to be considered for delivering a site-specific epigenetic modifying agent of the invention include, for example, biological stability of the epigenetic modifying agent, prevention of non-specific effects, and accumulation of the epigenetic modifying agent in the target tissue. The non-specific effects of an epigenetic modifying agent can be minimized by local administration, for example, by direct injection or implantation into a tissue or topically administering a composition comprising the epigenetic modifying agent. Local administration to a treatment site maximizes local concentration of the epigenetic modifying agent, limits the exposure of the epigenetic modifying agent to systemic tissues that can otherwise be harmed by the epigenetic modifying agent or that can degrade the epigenetic modifying agent, and permits a lower total dose of the epigenetic modifying agent to be administered.

For administering a site-specific epigenetic modifying agent systemically for the treatment of a disease, such as a disease or disorder associated with CXCL9, CXCL10, and/or CXCL11, e.g., liver disease, the epigenetic modifying agent, e.g., an epigenetic modifying agent comprising a site-specific targeting moiety comprising a nucleic acid molecule, can be modified or alternatively delivered using a drug delivery system; both methods act to prevent the rapid degradation of a site-specific targeting moiety comprising a nucleic acid molecule by endo- and exo-nucleases in vivo. Modification of an epigenetic modifying agent comprising a site-specific targeting moiety comprising a nucleic acid molecule or a pharmaceutical carrier also permits targeting of the epigenetic modifying agent to a target tissue and avoidance of undesirable off-target effects. For example, an epigenetic modifying agent of the invention may be modified by chemical conjugation to lipophilic groups such as cholesterol to enhance cellular uptake and prevent degradation.

Alternatively, an epigenetic modifying agent of the invention may be delivered using a drug delivery system such as a nanoparticle, a dendrimer, a polymer, a liposome, or a cationic delivery system. Positively charged cationic delivery systems facilitate binding of epigenetic modifying agent (e.g., negatively charged molecule) and also enhance interactions at the negatively charged cell membrane to permit efficient uptake of an epigenetic modifying agent by the cell. Cationic lipids, dendrimers, or polymers can either be bound to an epigenetic modifying agent, or induced to form a vesicle or micelle (see e.g., Kim S H. et al., (2008) Journal of Controlled Release 129 (2): 107-116) that encases the epigenetic modifying agent. The formation of vesicles or micelles further prevents degradation of the epigenetic modifying agent when administered systemically. Methods for making and administering cationic complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al. (2007) J. Hypertens. 25:197-205, which are incorporated herein by reference in their entirety). Some non-limiting examples of drug delivery systems useful for systemic delivery of an epigenetic modifying agent of the invention include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an epigenetic modifying agent (e.g., gRNA, or mRNA) forms a complex with cyclodextrin for systemic administration. Methods for administration and pharmaceutical compositions comprising cyclodextrins may be found in U.S. Pat. No. 7,427,605, the entire contents of which are incorporated herein by reference.

The epigenetic modifying agents of the invention may be incorporated into pharmaceutical compositions suitable for administration. Such compositions typically include one or more species of epigenetic modifying agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral, or parenteral. Parenteral administration includes intravenous drip, subcutaneous, intraperitoneal or intramuscular injection, or intrathecal or intraventricular administration.

The route and site of administration may be chosen to enhance delivery or targeting of the epigenetic modifying agent comprising a site-specific targeting moiety to a particular location. For example, to target cells including, but not limited to, liver cells, skin cells, heart cells, pancreas cells, pituitary gland cells, and connective tissue cells (e.g., tissues and cells found in joints), topical administration or subcutaneous injection, etc., may be used.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added.

Compositions for intravenous administration may include sterile aqueous solutions which may also contain buffers, diluents, and other suitable additives.

Formulations for parenteral administration may include sterile aqueous solutions which may also contain buffers, diluents, and other suitable additives. For intravenous use, the total concentration of solutes may be controlled to render the preparation isotonic.

In one embodiment, the administration of an epigenetic modifying agent composition of the invention is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral, or ocular. Administration can be provided by the subject or by another person, e.g., a health care provider. The composition may be provided in measured doses or in a dispenser which delivers a metered dose. Selected modes of delivery are discussed in more detail below.

In certain embodiments, the epigenetic modifying agents of the invention are polynucleotides, such as mRNAs, and are formulated in lipid nanoparticles (LNPs).

### Compositions Comprising a Site-Specific Epigenetic Modifying Agent of the Invention

The site-specific epigenetic modifying agents of the invention may be formulated into compositions, such as pharmaceutical compositions, using one or more excipients to: (1) increase stability; (2) increase cell transfection; (3) permit sustained or delayed release (e.g., from a depot formulation); (4) alter the biodistribution (e.g., target the epigenetic modifying agent to specific tissues or cell types); (5) increase the translation of an encoded protein in vivo; and/or (6) alter the release profile of an encoded protein in vivo. In addition to traditional excipients, such as any and all solvents, dispersion media, diluents, or other liquid vehicles, dispersion or suspension aids, surface active agents, isotonic agents, thickening or emulsifying agents, preservatives, excipients for use in the compositions of the invention may include, without limitation, lipidoids, liposomes, lipid nanoparticles, polymers, lipoplexes, core-shell nanoparticles, peptides, proteins, cells transfected with nucleic acid molecules, modified nucleic acid molecules, or RNA (e.g., for transplantation into a subject), hyaluronidase, nanoparticle mimics and combinations thereof. Accordingly, the pharmaceutical compositions of the invention can include one or more excipients, each in an amount that together increases the stability of the epigenetic modifying agent, increases cell transfection by the epigenetic modifying agent, increases the expression of modified nucleic acid, or mRNA encoded protein, and/or alters the release profile of an epigenetic modifying agent. Further, the epigenetic modifying agents of the present invention may be formulated using self-assembled nucleic acid nanoparticles (see, e.g., U.S. Patent Publication No. 2016/0038612A1, which is incorporated herein by reference in its entirety).

### Lipidoid

The synthesis of lipidoids has been extensively described and formulations containing these compounds are particularly suited for delivery of an epigenetic modifying agent of the invention, such as an epigenetic modifying agent comprising a site-specific targeting moiety comprising a nucleic acid molecule, e.g., comprising modified nucleic acid molecules or mRNA (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 201 0 107:1864-1869; Siegwart et al., Proc Natl Acad Sci USA. 2011108:12996-3001; the contents of all of which are incorporated herein in their entireties).

For example, lipidoids have been used to effectively deliver double stranded small interfering RNA molecules, single stranded nucleic acid molecules, modified nucleic acid molecules or modified mRNA. (See, e.g., US Patent Publication 2016/0038612A1). Complexes, micelles, liposomes or particles can be prepared containing these lipidoids and, therefore, provide effective delivery of a site-specific targeting moiety comprising a nucleic acid molecule, as judged by the production of an encoded protein, following the administration of a lipidoid formulation, e.g., via localized and/or systemic administration. Lipidoid complexes of can be administered by various means including, but not limited to, intravenous, intramuscular, intradermal, intraperitoneal or subcutaneous routes.

In vivo delivery of a site-specific targeting moiety comprising, e.g., a nucleic acid molecule, may be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with different lipidoids, including, but not limited to penta [3-(1-laury laminopropiony I)]-triethy lenetetramine hydrochloride (TETA-5LAP; aka 98NI2-5, see Murugaiah et al., Analytical Biochemistry, 401:61 (2010); the contents of which are herein incorporated by reference in their entirety), C12-200 (including derivatives and variants), and MDI, may be used.

In one embodiment, an epigenetic modifying agent comprising a site-specific targeting moiety comprising, e.g., a nucleic acid molecule, is formulated with a lipidoid for systemic intravenous administration to target cells of the liver. For example, a final optimized intravenous formulation comprising an epigenetic modifying agent comprising a site-specific targeting moiety comprising a nucleic acid molecule, and a lipid molar composition of 42% 98NI2-5, 48% cholesterol, and 10% PEG-lipid with a final weight ratio of about 7.5 to 1 total lipid to nucleic acid molecule, and a C14 alkyl chain length on the PEG lipid, with a mean particle size of roughly 50-60 nm, can result in the distribution of the formulation to be greater than 90% to cells including, but not limited to, liver cells, skin cells, heart cells, pancreas cells, pituitary gland cells, and connective tissue cells (e.g., tissues and cells found in joints). In another example, an intravenous formulation using a C12-200 lipidoid (see, e.g., PCT Publication No. WO 2010/129709, which is herein incorporated by reference in its entirety) having a molar ratio of 50/10/38.5/1.5 of C12-200/disteroylphosphatidyl choline/cholesteroI/PEG-DMG, with a weight ratio of 7 to 1 total lipid to nucleic acid molecule, and a mean particle size of 80 nm may be used to deliver an epigenetic modifying agent comprising a site-specific targeting moiety comprising a nucleic acid molecule, to cells including, but not limited to, liver cells, skin cells, heart cells, pancreas cells, pituitary gland cells, and connective tissue cells (e.g., tissues and cells found in joints). In another embodiment, an MDI lipidoid-containing formulation may be used to effectively deliver an epigenetic modifying agent comprising a site-specific targeting moiety comprising a nucleic acid molecule, to cells including, but not limited to, liver cells, skin cells, heart cells, pancreas cells, pituitary gland cells, and connective tissue cells (e.g., tissues and cells found in joints) in vivo. The characteristics of optimized lipidoid formulations for intramuscular or subcutaneous routes may vary significantly depending on the target cell type and the ability of formulations to diffuse through the extracellular matrix into the blood stream. Lipidoid-formulated nucleic acid molecules may be used to deliver the formulation to several cells types including, but not limited to cells including, liver cells, skin cells, heart cells, pancreas cells, pituitary gland cells, and connective tissue cells (e.g., tissues and cells found in joints). Different ratios of lipidoids and other components including, but not limited to, disteroylphosphatidyl choline, cholesterol and PEG-DMG, may be used to optimize the formulation for delivery to different cell types including, but not limited to cells including, liver cells, skin cells, heart cells, pancreas cells, pituitary gland cells, and connective tissue cells (e.g., tissues and cells found in joints), etc. For example, the component molar ratio may include, but is not limited to, 50% CI2-200, 10% disteroylphosphatidyl choline, 38.5% cholesterol, and 1.5% PEG-DMG (see Leuschner et al., Nat Biotechnol 2011 29:1005-101 0; the contents of which are herein incorporated by reference in its entirety). The use of lipidoid formulations for the localized delivery to cells (such as, but not limited to, adipose cells and muscle cells) via either subcutaneous, intradermal or intramuscular delivery, may not require all of the formulation components desired for systemic delivery and, as such, may comprise only the lipidoid and an epigenetic modifying agent comprising a site-specific targeting moiety comprising, e.g., a nucleic acid molecule, as described herein.

Combinations of different lipidoids may be used to improve the efficacy of the formulations by increasing cell transfection and/or increasing the translation of encoded protein contained therein (see Whitehead et al., Mol. Ther. 2011, 19:1688-1694, the contents of which are herein incorporated by reference in their entirety).

In one embodiment, the lipidoid may be prepared from the conjugate addition of alklamines to acrylates. As a non-limiting example, a lipidoid may be prepared by the methods described in PCT Patent Publication No. WO 2014/028487, the contents of which are herein incorporated by reference in its entirety. In one embodiment, the lipidoid may comprise a compound having formula (I), formula (II), formula (III), formula (IV) or formula (V) as described in PCT Patent Publication No. WO 2014/028487, the contents of which are herein incorporated by reference in their entirety. In one embodiment, the lipidoid may be biodegradable.

### Liposomes, Lipoplexes, and Lipid Nanoparticles

An epigenetic modifying agent of the invention may be formulated using one or more liposomes, lipoplexes, or lipid nanoparticles. In one embodiment, pharmaceutical compositions of the invention include liposomes. Liposomes are artificially-prepared vesicles which are primarily composed of a lipid bilayer and may be used as a delivery vehicle for the administration of nutrients and pharmaceutical formulations. Liposomes may be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes, such as synthetic membrane vesicles, may be prepared by the methods, apparatus and devices described in U.S. Patent Publication Nos. 2013/0177638, 2013/0177637, 2013/0177636, 201/30177635, 2013/0177634, 2013/0177633, 2013/0183375, 2013/0183373, 2013/0183372 and 2016/0038612) and PCT Patent Publication No WO 2008/042973, the contents of each of which are herein incorporated by reference in their entirety.

In one embodiment, a pharmaceutical composition described herein may include, without limitation, liposomes such as those formed from 1,2-dioleyloxy-N,N-dimethyl ami-nopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.). In one embodiment, a pharmaceutical composition described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 19996:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication Nos 2013/0122104, 2013/0303587, and 2016/0038612; the contents of each of which are incorporated herein in their entireties). The original manufacturing method of Wheeler et al. was a detergent dialysis method, which was later improved by Jeffs et al. and is referred to as the spontaneous vesicle formation method. The liposome formulations of the invention may be composed of 3 to 4 lipid components in addition an epigenetic modifying agent comprising a site-specific targeting moiety. As an example a liposome of the invention can contain, but is not limited to, 55% cholesterol, 20% disteroylphosphatidyl choline (DSPC), 10% PEG-SDSG, and 15% 1,2-dioleyloxy-N,N-dimethylaminopropane (DODMA), as described by Jeffs et al. As another example, liposome formulations of the invention may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethy laminopropane (DLenDMA), as described by Heyes et al. In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In another embodiment, formulations of the invention may comprise a percentage of cholesterol selected from the group consisting of 28.5%, 31.5%, 33.5%, 36.5%, 37.0%, 38.5%, 39.0% and 43.5%. In some embodiments, liposome formulations of the invention may comprise from about 5.0% to about 10.0% DSPC and/or from about 7.0% to about 15.0% DSPC.

In one embodiment, a pharmaceutical composition may include liposomes which may be formed to deliver an epigenetic modifying agent of the invention. The epigenetic modifying agent comprising a site-specific targeting moiety comprising may be encapsulated by the liposome and/or it may be contained in an aqueous core which may then be encapsulated by the liposome (see, e.g., PCT Patent Publication Nos. WO 2012/031046, WO 2012/031043, WO 2012/030901 and WO 2012/006378 and U.S. Patent Publication Nos. 2013/0189351, 2013/0195969 and 201/30202684, the contents of each of which are herein incorporated by reference in their entirety).

In another embodiment, liposomes for use in the present invention may be formulated for targeted delivery. As a non-limiting example, the liposome may be formulated for targeted delivery to cells including, but not limited to, liver cells, skin cells, heart cells, pancreas cells, pituitary gland cells, and connective tissue cells (e.g., tissues and cells found in joints), etc. Such a liposome may include, but is not limited to, a liposome described in U.S. Patent Publication No. 2013/0195967, the contents of which are herein incorporated by reference in its entirety.

In one embodiment, formulations comprising liposomes and an epigenetic modifying agent may be administered topically, subcutaneously, intramuscularly, intradermally, or intravenously.

In another embodiment, a lipid formulation of the invention may include at least one cationic lipid, a lipid which enhances transfection and a least one lipid which contains a hydrophilic head group linked to a lipid moiety (International Pub. No. WO2011076807 and U.S. Pub. No. 20110200582; the entire contents of each of which is herein incorporated by reference in their entirety). In another embodiment, a lipid formulation of the invention is a lipid vesicle which may have crosslinks between functionalized lipid bilayers (see U.S. Patent Publication No. 2012/0177724, the contents of which are herein incorporated by reference in their entirety).

In one embodiment, a formulation comprising an epigenetic modifying agent is a lipid nanoparticle (LNP) which may comprise at least one lipid. The lipid may be selected from, but is not limited to, DLin-DMA, DLin-K-DMA, 98NI2-5, CI2-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. 2013/0150625.

In one embodiment, the cationic lipid may be selected from, but not limited to, a cationic lipid described in PCT Publication Nos. WO 2012/040184, WO 2011/153120, WO 2011/149733, WO 2011/090965, WO 2011/043913, WO 2011/022460, WO 2012/061259, WO 2012/054365, WO 2012/044638, WO 2010/080724, WO 2010/21865, WO 2008/103276, WO 2013/086373 and WO 2013/086354, U.S. Pat. Nos. 7,893,302, 7,404,969, 8,283, 333, 8,466,122 and 8,569,256, and U.S. Patent Publication Nos. 2010/0036115, 2012/0202871, 2013/0064894, 2013/0129785, 2013/0150625, 2013/0178541, 2013/0225836 and 2014/0039032; the contents of each of which are herein incorporated by reference in their entirety. In another embodiment, the cationic lipid may be selected from, but not limited to, formula A described in PCT Publication Nos. WO 2012/040184, WO 0111/53120, WO 2011/149733, WO 2011/090965, WO 2011/043913, WO 2011/022460, WO 2012/061259, WO 2012/054365, WO 2012/044638 and WO 2013/116126 or U.S. Patent Publication Nos. 2013/0178541 and 2013/0225836; the contents of each of which is herein incorporated by reference in their entirety. In yet another embodiment, the cationic lipid may be selected from, but not limited to, formula CLI-CLXXIX of PCT Publication No. WO 2008/103276, formula CLICLXXIX of U.S. Pat. No. 7,893,302, formula CLICLXXXXII of U.S. Pat. No. 7,404,969 and formula I-VI of us Patent Publication No. 2010/0036115, formula I of U.S. Patent Publication No 2013/0123338; each of which is herein incorporated by reference in their entirety.

In one embodiment, the cationic lipid may be synthesized by methods known in the art and/or as described in PCT Publication Nos. WO 2012/040184 WO 2011/153120, WO 2011/149733, WO 2011/090965: WO 2011/043913, WO 2011/022460, WO 2012/061259, WO 2012/054365, WO 2012/044638, WO 2010/080724, WO 2010/21865, WO 2013/126803, WO 2013/086373, and WO 2013/086354; the contents of each of which are herein incorporated by reference in their entirety.

In one embodiment, the lipids which may be used in the formulations and/or for delivery of the epigenetic modifying agents described herein may be a cleavable lipid. As a non-limiting example, a cleavable lipid and/or pharmaceutical compositions comprising cleavable lipids include those described in PCT Patent Publication No. WO 2012/170889, the contents of which are herein incorporated by reference in their entirety. As another non-limiting example, the cleavable lipid may be HGT4001, HGT4002, HGT4003, HGT4004 and/or HGT4005 as described in PCT Patent Publication No. WO 2012/170889, the contents of which are herein incorporated by reference in their entirety.

In one embodiment, polymers which may be used in the formulation and/or delivery of the epigenetic modifying agents described herein may include, but is not limited to, poly(ethylene) glycol (PEG), polyethylenimine (PEI), dithiobis(succinimidylpropionate) (DSP), Dimethy 1-3,3′-dithiobispropionimidate (DTBP), poly(ethylene imine) biscarbamate (PEIC), poly(L-lysine) (PLL), histidine modified PLL, poly(N-vinylpyrrohdone) (PVP), poly(propylenimine (PPI), poly(amidoamine) (PAMAM), poly(amido ethylenimine) (SS-PAEI), triehtylenetetramine (TETA), poly(β-aminoester), poly(4-hydroxy-L-proine ester) (PHP), poly(allylamine), poly(α-[4-aminobutyl]-L-glycolic acid (PAGA), Poly(D,L-lactic-coglycolid acid (PLGA), Poly(N-ethyl-4-vinylpyridinium bromide), poly(phosphazene) s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), Chitosan, galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), Poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histone modified PLL SSPAEI, poly(β-aminoester), PHP, PAGA, PLGA, PPZ, PPE, PPA and PPE-EA.

In one embodiment, an LNP formulation of the invention may be prepared according to the methods described in PCT Publication Nos. WO 2011/127255 or WO 2008/103276, the contents of each of which are herein incorporated by reference in their entirety. As a non-limiting example, an epigenetic modifying agent comprising a site-specific targeting moiety may be encapsulated in an LNP formulation as described in PCT Publication Nos. WO 2011/127255 and/or WO 2008/103276; the contents of each of which are herein incorporated by reference in their entirety. As another non-limiting example, an epigenetic modifying agent comprising a site-specific targeting moiety as described herein, may be formulated in a nanoparticle to be delivered by a parenteral route as described in U.S. Patent Publication No. 2012/0207845 and PCT Publication No. WO 2014/008334; the contents of each of which are herein incorporated by reference in their entirety.

In one embodiment, LNP formulations described herein may be administered intramusculary. The LNP formulation may comprise a cationic lipid described herein, such as, but not limited to, DLin-DMA, DLin-KC2-DMA, DLin-MC3-DMA, DODMA and C12-200.

In one embodiment, LNP formulations described herein comprising an epigenetic modifying agent as described herein, may be administered intradermally. The LNP formulation may comprise a cationic lipid described herein, such as, but not limited to, DLin-DMA, DLin-KC2-DMA, DLin-MC3-DMA, DODMA and C12-200.

The nanoparticle formulations may comprise conjugate, such as a phosphate conjugate, a polymer conjugates, a conjugate that enhances the delivery of nanoparticle as described in US Patent Publication No. US20160038612 A1.

In one embodiment, the lipid nanoparticle formulation comprises DLin-MC3-DMA as described in US Patent Publication No. US20100324120.

In one embodiment, the lipid nanoparticle comprises a lipid compound, or a pharmaceutically acceptable salt, tautomer or stereoisomer thereof, or a lipid nanoparticle formulation, as described in US Patent No.: U.S. Pat. No. 10,723,692B2, US Patent Publication Nos. US20200172472A1, US20200163878A1, US20200046838A1, US20190359556A1, US20190314524A1, US20190274968A1, US20190022247A1, US20180303925A1, US20180185516A1, US20160317676A1, International Patent Publication No.: WO20200146805A1, WO2020081938A1, WO2019089828A1, WO2019036030A1, WO2019036028A1, WO2019036008A1, WO 2018200943A1, WO2018191719A1, WO2018107026A1, WO2018081480A1, the contents of each of which are herein incorporated by reference in their entirety (Acuitas Therapeutics, Inc.).

In one embodiment, the lipid nanoparticle comprises an amino lipid, or a pharmaceutically acceptable salt, tautomer or stereoisomer thereof, or a lipid nanoparticle formulation, described by Tekmira Pharmaceuticals Corp. in U.S. Pat. No. 9,139,554B2, U.S. Pat. No. 9,051,567B2, U.S. Pat. No. 8,883,203B2, US Patent Publication US20110117125A1, the contents of each of which are herein incorporated by reference in their entirety. In one particular example, the compound described in U.S. Pat. No. 9,139,554B2 is DLin-kC2-DMA.

In one embodiment, the lipid nanoparticle comprises an amino lipid, or a pharmaceutically acceptable salt, tautomer or stereoisomer thereof, or a lipid nanoparticle formulation, described by Arbutus Biopharma Corp. in U.S. Pat. No. 10,561,732B2, U.S. Pat. No. 9,938,236B2, U.S. Pat. No. 9,687,550B2, US Patent Publication US20190240354A1, US20170027658A1, WO2020097493A1, WO2020097520A1, WO2020097540A1, WO2020097548A1, the contents of each of which are herein incorporated by reference in their entirety.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosla tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104 (5): 1482-487; Lai et al. Adv Drug Deliv Rev. 200961 (2): 158-171; the contents of each of which are herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in their entirety.

In one embodiment, an epigenetic modifying agent comprising a site-specific targeting moiety as described herein, is formulated as a lipoplex, such as, without limitation, the ATUPLEX™ system, the DACC system, the DBTC system and other siRNAlipoplex technology from Silence Therapeutics (London, United Kingdom), STEMFECFM from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 200613:1360-1370; Gutbier et al., PulmPharmacol. Ther. 201023:334-344; Kaufmann et al. Microvasc Res 2010 80:286-293; Weide et al. J Immunother. 2009 32:498-507; Weide et al. J Immnnother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005,23:709-717; Peer et al., Proc NatlAcad Sci USA. 2007 6; 104:4095-4100; de Fougerolles Hum Gene Ther. 2008 19:125-132; all of which are incorporated herein by reference in their entirety).

In one embodiment such formulations may also be constructed or compositions altered such that they passively or actively are directed to different cell types in vivo, for example, epithelial cells (Akinc et al. Mol Ther. 2010 18:1357-1364; Song et al., Nat Biotechnol. 2005 23:709-717; Judge et al., J ClinInvest. 2009 119:661-673; Kaufmann et al., Microvasc Res 2010 80:286-293; Santel et al., Gene Ther 200613:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 20085:25-44; Peer et al., Science. 2008 319:627-630; Peer and Lieberman, Gene Ther. 2011 18:1127-1133; all of which are incorporated herein by reference in its entirety). Formulations can also be selectively targeted through expression of different ligands on their surface as exemplified by, but not limited by, folate, transferrin, N-acetylgalactosamine (GaINAc), and antibody targeted approaches (Kolhatkar et al., Curr Drug Discov Technol. 2011 8:197-206; Musacchio and Torchilin, Front Biosci. 201116:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319;Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; the contents of all of which are incorporated herein by reference in its entirety).

In one embodiment, an epigenetic modifying agent comprising a site-specific targeting moiety of the invention, may be formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; herein incorporated by reference in its entirety). As a non-limiting example, the SLN may be the SLN described in PCT Publication No. WO2013/105101, the contents of which are herein incorporated by reference in their entirety. As another non-limiting example, the SLN may be made by the methods or processes described in PCT Publication No. WO 2013/105101, the contents of which are herein incorporated by reference in their entirety.

Liposomes, lipoplexes, or lipid nanoparticles may be used to improve the efficacy of an epigenetic modifying agent comprising a site-specific targeting moiety comprising, e.g., a nucleic acid molecule, to direct protein production as these formulations may be able to increase cell transfection by a nucleic acid molecule; and/or increase the translation of encoded protein (e.g., an effector of the invention). One such example involves the use of lipid encapsulation to enable the effective systemic delivery of polyplex plasmid DNA (Heyes et al., Mol Ther. 2007 15:713-720; the contents of which are herein incorporated by reference in its entirety). The liposomes, lipoplexes, or lipid nanoparticles of the invention may also increase the stability of an epigenetic modifying agent comprising a site-specific targeting moiety comprising, e.g., a nucleic acid molecule. Liposomes, lipoplexes, or lipid nanoparticles are described in U.S. Patent Publication No. 2016/0038612, the contents of which are incorporated herein by reference in their entirety.

In one embodiment, an epigenetic modifying agent comprising a site-specific targeting moiety comprising may be formulated for controlled release and/or targeted delivery. As used herein, “controlled release” refers to a pharmaceutical composition or compound release profile that conforms to a particular pattern of release to effect a therapeutic outcome. In one embodiment, an epigenetic modifying agent comprising a site-specific targeting moiety, as described herein, may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or epigenetic modifying agent of the invention may be enclosed, surrounded or encased within the delivery agent. “Partial encapsulation” or “partially encapsulated” means that less than 10, 10, 20, 30, 40 50 or less of the pharmaceutical composition or epigenetic modifying agent of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or epigenetic modifying agent of the invention are encapsulated in the delivery agent.

In one embodiment, an epigenetic modifying agent comprising a site-specific targeting moiety comprising as described herein, may be encapsulated in a therapeutic nanoparticle. Therapeutic nanoparticles may be formulated by methods described herein and known in the art such as, but not limited to, PCT Publication Nos. WO 2010/005740, WO 2010/030763, WO 2010/005721, WO 2010/005723, WO 2012/054923, U.S. Patent Publication Nos. 2201/10262491, 2010/0104645, 2010/0087337, 2010/0068285, 2011/0274759, 2010/0068286, 2012/0288541, 2013/0123351, 2013/0230567, 2013/0236500, 2013/0302433, 2013/0302432, 1013/0280339 and 2013/0251757, and U.S. Pat. Nos. 8,206,747, 8,293,276 8,318,208, 8,318,211, 8,623,417, 8,617,608, 8,613,954, 8,613,951, 8,609,142, 8,603,534 and 8,563,041; the contents of each of which is herein incorporated by reference in their entirety. In another embodiment, therapeutic polymer nanoparticles may be prepared by the methods described in U.S. Patent Publication No. 2012/0140790, herein incorporated by reference in its entirety. As a non-limiting example, the therapeutic nanoparticle may comprise about 4 to about 25 weight percent of an epigenetic modifying agent and about 10 to about 99 weight percent of a diblock poly(lactic) acid-poly(ethylene) glycol copolymer comprising poly(lactic) acid as described in US Patent Publication No. 2013/0236500, the contents of which are herein incorporated by reference in its entirety. As another non-limiting example, the nanoparticle may comprise about 0.2 to about 35 weight percent of an epigenetic modifying agent and about 10 to about 99 weight percent of a diblock poly(lactic) acid-poly(ethylene)glycol copolymer as described in U.S. Patent Publication Nos. 2013/0280339 and 2010251757 and U.S. Pat. No. 8,652,528, the contents of each of which are herein incorporated by reference in their entirety.

In one embodiment, an epigenetic modifying agent formulated in therapeutic nanoparticles may be administered intramuscularly, intradermally, or intravenously.

In one embodiment, an epigenetic modifying agent formulated in ACCURINS™ nanoparticles may be administered intramuscularly, intrademrally, or intravenously.

In one embodiment, an epigenetic modifying agent may be delivered in therapeutic nanoparticles having a high glass transition temperature such as, but not limited to, the nanoparticles described in US Patent Publication Nos. 2014/0030351 and 2011/0294717, the entire contents of each of which are incorporated herein by reference.

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a nonlimiting example, the sustained release nanoparticle may comprise a polymer and an epigenetic modifying agent of the present invention (see PCT Publication No. WO2010075072 and U.S. Patent Publication Nos. 2010/0216804, 2011/0217377, 2012/0201859, 2013/0243848 and 2013/0243827, each of which is herein incorporated by reference in their entirety).

In one embodiment, an epigenetic modifying agent of the invention may be encapsulated in, linked to and/or associated with synthetic nanocarriers. Synthetic nanocarriers include, but are not limited to, those described in PCT Publication. Nos. WO 2010/005740, WO 2010/030763, WO 2012/13501, WO 2012/149252, WO 2012149255, WO 2012149259, WO 2012149265, WO 2012149268, WO 2012149282, WO 2012149301, WO 2012149393, WO 2012149405, WO 2012149411 and WO 2012149454 and US Patent Publication Nos. 20110262491, 20100104645, 20100087337, 20120244222 and US20130236533, and U.S. Pat. No. 8,652,487, the contents of each of which is herein incorporated by reference in their entirety. The synthetic nanocarriers may be formulated using methods known in the art and/or described herein. As a nonlimiting example, the synthetic nanocarriers may be formulated by the methods described in PCT Publication Nos. WO 2010005740, WO 2010030763 and WO 201213501 and US Patent Publication Nos. 20110262491, 20100104645, 20100087337 and 20120244222, each of which is herein incorporated by reference in their entirety. In another embodiment, the synthetic nanocarrier formulations may be lyophilized by methods described in PCT Publication No. WO 2011072218 and U.S. Pat. No. 8,211,473; each of which is herein incorporated by reference in their entirety. In yet another embodiment, formulations of the present invention, including, but not limited to, synthetic nanocarriers, may be lyophilized or reconstituted by the methods described in US Patent Publication No. 20130230568, the contents of which are herein incorporated by reference in its entirety.

In one embodiment, synthetic nanocarriers comprising an epigenetic modifying agent may be administered intramuscularly, intradermally, or intravenously.

In some embodiments, an epigenetic modifying agent may be formulated for delivery using smaller LNPs. Such particles may comprise a diameter from below 0.1 μm up to 1000 μm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm.

In another embodiment, an epigenetic modifying agent may be formulated for delivery using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nm, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In one embodiment, an epigenetic modifying agent may be formulated in smaller LNPs and may be administered intramuscularly, intradermally, or intravenously.

In one embodiment, an epigenetic modifying agent may be formulated for delivery using the drug encapsulating microspheres described in PCT Patent Publication No. WO 2013063468 or U.S. Pat. No. 8,440,614, each of which is herein incorporated by reference in its entirety. In another aspect, the amino acid, peptide, polypeptide, lipids (APPL) are useful in delivering the epigenetic modifying agents of the invention to cells (see PCT Patent Publication No. WO 2013063468, herein incorporated by reference in its entirety).

In one aspect, the lipid nanoparticle may be a limit size lipid nanoparticle described in PCT Patent Publication No. WO 2013059922, herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and I-palmitoyl-2-oleoyl phosphatidylcholine (POPC). In another aspect the limit size lipid nanoparticle may comprise a polyethylene glycol-lipid such as, but not limited to, DLPEPEG, DMPE-PEG, DPPC-PEG and DSPE-PEG.

In one embodiment, an epigenetic modifying agent of the invention may be delivered, localized and/or concentrated in a specific location using the delivery methods described in PCT Patent Publication No. WO 2013063530, the contents of which are herein incorporated by reference in its entirety. As a non-limiting example, a subject may be administered an empty polymeric particle prior to, simultaneously with or after delivering the epigenetic modifying agent to the subject. The empty polymeric particle undergoes a change in volume once in contact with the subject and becomes lodged, embedded, immobilized or entrapped at a specific location in the subject.

In one embodiment, an epigenetic modifying agent may be formulated in an active substance release system (See e.g., US Patent Publication No. 20130102545, herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., an epigenetic modifying agent of the invention), where the therapeutically active substance is released by the cleavage of the substrate molecule by the catalytically active nucleic acid.

In one embodiment, the nanoparticles of the present invention may be water soluble nanoparticles such as, but not limited to, those described in PCT Publication No. WO 2013090601, the contents of which are herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In one embodiment, the nanoparticles of the present invention are stealth nanoparticles or target-specific stealth nanoparticles such as, but not limited to, those described in U.S. Patent Publication Nos. 20130172406 (Bind), 20130251817 (Bind), 2013251816 (Bind) and 20130251766 (Bind), the contents of each of which are herein incorporated by reference in its entirety. The stealth nanoparticles may comprise a diblock copolymer and a chemotherapeutic agent. These stealth nanoparticles may be made by the methods described in us Patent Publication Nos. 20130172406, 20130251817, 2013251816 and 20130251766, the contents of each of which are herein incorporated by reference in its entirety. As a non-limiting example, the stealth nanoparticles may target cancer cells such as the nanoparticles described in US Patent Publication Nos. 20130172406, 20130251817, 2013251816 and 20130251766, the contents of each of which are herein incorporated by reference in its entirety.

In one embodiment, stealth nanoparticles comprising an epigenetic modifying agent of the invention may be administered intramuscularly, intradermally, or intravenously.

In one embodiment, an epigenetic modifying agent of the invention may be formulated in and/or delivered in a lipid nanoparticle comprising a plurality of cationic lipids such as, but not limited to, the lipid nanoparticles described in US Patent Publication No. 20130017223, the contents of which are herein incorporated by reference in its entirety. As a non-limiting example, the LNP formulation may comprise a first cationic lipid and a second cationic lipid. As another non-limiting example, the LNP formulation may comprise DLin-MC2-DMA and DLinMC4-DMA. As yet another non-limiting example, the LNP formulation may comprise DLin-MC3-DMA and CI2-200. In one embodiment, the LNP formulations comprising a plurality of cationic lipids (such as, but not limited to, those described in US Patent Publication No. US20130017223, the contents of which are herein incorporated by reference in its entirety) and may be administered intramuscularly, intradermally, or intravenously.

In one embodiment, an epigenetic modifying agent as described herein, may be formulated in and/or delivered in a lipid nanoparticle comprising the cationic lipid DLin-MC3-DMA and the neutral lipid DOPE. The lipid nanoparticle may also comprise a PEG based lipid and a cholesterol or antioxidant. These lipid nanoparticle formulations comprising DLin-MC3-DMA and DOPE and an epigenetic modifying agent may be administered intramuscularly, intradermally, or intravenously.

In one embodiment, the lipid nanoparticle comprising DLin-MC3-DMA and DOPE may comprise a PEG lipid such as, but not limited to, pentaerythritol PEG ester tetrasuccinimidyl and pentaerythritol PEG ether tetra-thiol, PEGc-DOMG, PEG-DMG (1,2-Dimyristoyl-sn-glycerol, methoxypolyethylene Glycol), PEG-DSG (1,2-Distearoyl-snglycerol, methoxypolyethylene Glycol), PEG-DPG (1,2-Dipalmitoyl-sn-glycerol, methoxypolyethylene glycol), PEG-DSA (PEG coupled to 1,2-distearyloxypropyl-3-amine), PEG-DMA (PEG coupled to 1,2-dimyristyloxypropyl-3-amine, PEG-c-DNA, PEG-c-DMA, PEG-S-DSG, PEG-c-DMA, PEG-DPG, PEG-DMG 2000 and those described herein and/or known in the art.

In one embodiment, the lipid nanoparticle comprising DLin-MC3-DMA and DOPE may include 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of a PEG lipid.

In one embodiment, the lipid nanoparticle comprising DLin-MC3-DMA and DOPE may include 25.0% cholesterol to about 50.0% cholesterol, from about 30.0% cholesterol to about 45.0% cholesterol, from about 35.0% cholesterol to about 50.0% cholesterol and/or from about 48.5% cholesterol to about 60% cholesterol. In one embodiment, formulations may comprise a percentage of cholesterol selected from the group consisting of 28.5%, 31.5%, 33.5%, 36.5%, 37.0%, 38.5%, 39.0%, 43.5% and 48.5%.

In one embodiment, the lipid nanoparticle comprising DLin-MC3-DMA and DOPE may include 25.0% antioxidant to about 50.0% antioxidant, from about 30.0% antioxidant to about 45.0% antioxidant, from about 35.0% antioxidant to about 50.0% antioxidant and/or from about 48.5% antioxidant to about 60% antioxidant. In one embodiment, formulations may comprise a percentage of antioxidant selected from the group consisting of 28.5%, 31.5%, 33.5%, 36.5%, 37.0%, 38.5%, 39.0%, 43.5% and 48.5%.

The epigenetic modifying agent of the invention can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY® (Seattle, Wash.), DMRIIDOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.).

The polymer formulations may permit the sustained or delayed release of an epigenetic modifying agent (e.g., following intramuscular, intradermal or subcutaneous injection). The altered release profile of the epigenetic modifying agent can result in, for example, translation of an encoded protein over an extended period of time. The polymer formulation may also be used to increase the stability of the epigenetic modifying agent. For example, biodegradable polymers have been previously used to protect nucleic acids other than modified mRNA from degradation and been shown to result in sustained release of payloads in vivo (Rozema et al., Proc Natl Acad Sci USA. 2007 104:12982-12887; Sullivan et al., Expert Opin Drug Deliv. 2010 7:1433-1446; Convertine et al., Biomacromolecules. 2010 Oct. 1; Chu et al., Acc Chern Res. 2012 Jan. 13; Manganiello et al., Biomaterials. 2012 33:2301-2309; Benoit et al., Biomacromolecules. 2011 12:2708-2714; Singha et al., Nucleic Acid Ther. 2011 2:133-147; deFougerolles Hum Gene Ther. 2008 19:125-132; Schaffert and Wagner, Gene Ther. 2008 16:1131-1138; Chaturvedi et al., Expert Opin Drug Deliv. 2011 8:1455-1468; Davis, Mol Pharm. 2009 6:659-668; Davis, Nature 201 0464:1067-1070; each of which is herein incorporated by reference in its entirety).

In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethic on Inc. Cornelia, Ga.), TISSELL® (Baxter International, Inc Deerfield, Ill.), PEG-based sealants, and COSEAL® (Baxter International, Inc Deerfield, Ill.).

### Vector Encoded Site-Specific Epigenetic Modifying Agents of the Invention

Epigenetic modifying agents comprising a site-specific targeting moiety, e.g., comprising a nucleic acid molecule, may be expressed from transcription units inserted into DNA or RNA vectors (see, e.g., Couture, A, et al., TIG. (1996), 12:5-10; WO 00/22113, WO 00/22114, and U.S. Pat. No. 6,054,299). In some embodiment, expression is sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (see, Gassmann, et al., (1995) Proc. Natl. Acad. Sci. USA 92:1292). Different components of the epigenetic modifying agent, e.g., gRNA and effector, can be located on separate expression vectors that can be co-introduced (e.g., by transfection or infection) into a target cell. Alternatively, each individual component can be transcribed by promoters both of which are located on the same expression plasmid.

Delivery of an epigenetic modifying agent expressing vector can be systemic, such as by intravenous or intramuscular administration, by administration to target cells ex-planted from the patient followed by reintroduction into the patient, or by any other means that allows for introduction into a desired target cell.

In certain embodiment, the nucleic acids described herein or the nucleic acids encoding a protein described herein, e.g., an effector, are incorporated into a vector, e.g., a viral vector.

The individual strand or strands of an epigenetic modifying agent comprising a site-specific targeting moiety comprising a nucleic acid molecule can be transcribed from a promoter in an expression vector. Where two separate strands are to be expressed to generate, for example, a dsRNA, two separate expression vectors can be co-introduced (e.g., by transfection or infection) into a target cell. Alternatively, each individual strand of a nucleic acid molecule can be transcribed by promoters both of which are located on the same expression plasmid. In one embodiment, a nucleic acid molecule is expressed as inverted repeat polynucleotides joined by a linker polynucleotide sequence such that the nucleic acid molecule has a stem and loop structure.

Expression vectors are generally DNA plasmids or viral vectors. Expression vectors compatible with eukaryotic cells, preferably those compatible with vertebrate cells, can be used to produce recombinant constructs for the expression of an epigenetic modifying agent as described herein.

Constructs for the recombinant expression of an epigenetic modifying agent will generally require regulatory elements, e.g., promoters, enhancers, etc., to ensure the expression of the epigenetic modifying agent in target cells.

Expression of natural or synthetic nucleic acids is typically achieved by operably linking a nucleic acid encoding the nucleic acid of interest to a regulatory region, such as a promoter, and incorporating the construct into an expression vector. The vectors can be suitable for replication and integration in eukaryotes.

Regulatory regions, such as a promoter, suitable for operable linking to a nucleic acid molecules can be operably linked to a regulatory region such as a promoter. can be from any species. Any type of promoter can be operably linked to a nucleic acid sequence. Examples of promoters include, without limitation, tissue-specific promoters, constitutive promoters, and promoters responsive or unresponsive to a particular stimulus (e.g., inducible promoters). Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the present invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Additional regulatory regions that may be useful in nucleic acid constructs, include, but are not limited to, transcription and translation terminators, initiation sequences, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Useful selectable markers include, for example, antibiotic-resistance genes, such as neo and the like. Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

Signal peptides may also be included and can be used such that an encoded polypeptide is directed to a particular cellular location (e.g., the cell surface).

Reporter genes may be used for identifying potentially transfected cells and for evaluating the functionality of transcriptional control sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Other aspects to consider for vectors and constructs are known in the art.

In some embodiments, a vector, e.g., a viral vector comprises an epigenetic modifying agent comprising a site-specific targeting moiety comprising a nucleic acid molecule.

Viral vector systems which can be utilized with the methods and compositions described herein include, but are not limited to, (a) adenovirus vectors (e.g., an Ad5/F35 vector); (b) retrovirus vectors, including but not limited to lentiviral vectors (including integration competent or integration-defective lentiviral vectors), moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. See, e.g., U.S. Pat. Nos. 6,534,261; 6,607,882; 6,824,978; 6,933,113; 6,979,539; 7,013,219; and 7,163,824, the entire contents of each of which is incorporated by reference herein.

Vectors, including those derived from retroviruses such as adenoviruses and adeno-associated viruses and lentiviruses, are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector. Viral vector technology is well known in the art, and described in a variety of virology and molecular biology manuals.

In one embodiment, a suitable viral vector for use in the present invention is an adeno-associated viral vector, such as a recombinant adeno-associate viral vector.

Recombinant adeno-associated virus vectors (rAAV) are gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). AAV serotypes, including AAV1, AAV2, AAV3, AAV4, AAV5, AAV6, AAV7, AAV8 and AAV9, can be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in the liver (e.g., to cells including, but not limited to, liver cells, skin cells, heart cells, pancreas cells, pituitary gland cells, and connective tissue cells (e.g., tissues and cells found in joints)). Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and w2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

### Methods of the Invention

Reduction of Expression of CXCL9, CXCL10, and/or CXCL11 in a Cell

The present invention also provides methods of use of the agents and compositions described herein to reduce expression of CXCL9, CXCL10, and/or CXCL11 in a cell. The methods include contacting the cell (e.g., liver cell) with a site-specific epigenetic modifying agent, the epigenetic modifying agent comprising a site-specific targeting moiety which targets an expression control region associated with CXCL9, CXCL10, and/or CXCL11, and an effector molecule, thereby reducing expression of CXCL9, CXCL10, and/or CXCL11 in the cell. The site-specific epigenetic modifying agent, the effector, or both the site-specific epigenetic modifying agent and the effector may be present in a composition, such as a composition described above. In some embodiments, the site-specific epigenetic modifying agent and the effector are present in the same compositions. In other embodiments, the site-specific epigenetic modifying agent and the effector are present in different compositions. In some embodiments, the methods of the invention include contacting a cell with two site-specific epigenetic modifying agent (a first and a second agent). The two site specific epigenetic modifying agents may be present in the same composition, e.g., pharmaceutical composition, e.g., pharmaceutical composition comprising an LNP, or in separate compositions, e.g., pharmaceutical composition, e.g., pharmaceutical composition comprising an LNP. The cell may be contacted with the first site specific epigenetic modifying agent at one time and contacted with the second site specific epigenetic modifying agent at a second time, or the cell may be contacted with both agents at the same time.

In some embodiments, the methods of the invention include contacting a cell with two site-specific epigenetic modifying agent (a first and a second agent) which are encoded on the same nucleic acid molecule. In some embodiments, the first and second agent target the same gene, e.g., CXCL9, CXCL10, or CXCL11 gene. For example, the first agent targets CXCL9 and the second agent targets CXCL9. In some embodiments, the first and second agent targeting the same gene, e.g., CXCL9, CXCL10, or CXCL11, target different expression control elements associated with the gene which the first and second agent are targeting. In some embodiments, the first and second agent target a different gene, e.g., CXCL9, CXCL10, and/or CXCL11 gene. For example, the first agent targets CXCL9 and the second agent targets CXCL10.

In some embodiments, the methods of the invention include contacting a cell with three site-specific epigenetic modifying agent (a first, a second, and a third agent). The three site specific epigenetic modifying agents may be present in the same composition, e.g., pharmaceutical composition, e.g., pharmaceutical composition comprising an LNP, or in separate compositions, e.g., pharmaceutical composition, e.g., pharmaceutical composition comprising an LNP. The cell may be contacted with the first site specific epigenetic modifying agent at one time and contacted with the second site specific epigenetic modifying agent at a second time and contacted with the third site specific epigenetic modifying agent at a third time. In some embodiments, the cell may be contacted with two at the same time and a third agent at a second time. In some embodiments, the cell may be contacted with three agents at the same time.

Expression of CXCL9, CXCL10, and/or CXCL11 may be reduced as compared to, for example, a cell that was not contacted with the site-specific epigenetic modifying agent. Modulation in gene expression can be assessed by any methods known in the art. For example, a modulation in the expression may be determined by determining the mRNA expression level of a gene, e.g., in a cell, a plurality of cells, and/or a tissue sample, using methods routine to one of ordinary skill in the art, e.g., northern blotting, qRT-PCR; by determining the protein level of a gene using methods routine to one of ordinary skill in the art, such as western blotting, immunological techniques.

The term “reduced” in the context of the level of CXCL9, CXCL10, and/or CXCL11 gene expression or CXCL9, CXCL10, and/or CXCL11 protein production in a subject, or a disease marker or symptom refers to a statistically significant decrease in such level. The decrease can be, for example, at least 20%, 25%, 30%, 35%, 40%, 45%, 50%, 55%, 60%, 65%, 70%, 75%, 80%, 85%, 90%, or 95%, or below the level of detection for the detection method. In certain embodiments, the expression of the target is normalized, i.e., decreased towards or to a level accepted as within the range of normal for an individual without such disorder. As used here, “lower” in a subject can refer to lowering of gene expression or protein production in a cell in a subject does not require lowering of expression in all cells or tissues of a subject. For example, as used herein, lowering in a subject can include lowering of gene expression or protein production in the cells including, but not limited to, liver cells, skin cells, heart cells, pancreas cells, pituitary gland cells, and connective tissue cells (e.g., tissues and cells found in joints) of a subject.

The term “reduced” can also be used in association with normalizing a symptom of a disease or condition, i.e. decreasing the difference between a level in a subject suffering from a disease or disorder associated with CXCL9, CXCL10, and/or CXCL11, e.g., liver disease, towards or to a level in a normal subject not suffering from a disease or disorder associated with CXCL9, CXCL10, and/or CXCL11, e.g., liver disease. As used herein, if a disease is associated with an elevated value for a symptom, “normal” is considered to be the upper limit of normal. If a disease is associated with a decreased value for a symptom, “normal” is considered to be the lower limit of normal.

In some embodiments, a suitable cell for use in the methods of the invention is a mammalian cell. In some embodiments, the cell is a somatic cell. In some embodiments, the cell is a primary cell. For example, in some embodiments, the cell is a mammalian somatic cell. In some embodiments, the mammalian somatic cell is a primary cell. In some embodiments, the mammalian somatic cell is a non-embryonic cell.

### In Vitro Methods of the Invention

The step of contacting may be performed in vitro, in vivo (i.e., the cell may be within a subject), or ex vivo. In some embodiments, contacting a cell is performed ex vivo and the methods further include, prior to the step of contacting, a step of removing the cell (e.g., a mammalian cell) from a subject. In some embodiments, the methods further comprise, after the step of contacting, a step of (b) administering the cell (e.g., mammalian cells) to a subject.

In some embodiments, the cell is a liver cell. In some embodiments, the cell is contacted with a site-specific epigenetic modifying agent. In some embodiments, the site-specific epigenetic modifying agent is a repressor of CXCL9, CXCL10, and/or CXCL11.

In some embodiments, the cell contacted with the epigenetic modifying agent can be used ex vivo to determine repression of CXCL9, CXCL10, and/or CXCL11.

### In Vivo Methods of the Invention

The in vivo methods of the invention may include administering to a subject an agent or composition, or cells of the invention.

The term “subject,” as used herein refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject had a disease or a condition. In some embodiments, the subject is suffering from a disease, disorder or condition, e.g., a disease, disorder or condition that can be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Subjects that would benefit from the methods of the invention include subjects having an “a disease or disorder associated with CXCL9, CXCL10, and/or CXCL11” or a subject at risk of an “a disease or disorder associated with CXCL9, CXCL10, and/or CXCL11.”

Thus, the present invention further provides methods of treatment of a subject in need thereof. The treatment methods of the invention include administering an agent or composition of the invention to a subject, e.g., a subject that would benefit from a reduction of CXCL9, CXCL10, and/or CXCL11 expression, such as a subject having a disease or disorder associated with CXCL9, CXCL10, and/or CXCL11, e.g., liver disease, in a therapeutically effective amount. In some embodiments, the methods of the invention include the subject may be administered two site-specific epigenetic modifying agents (a first and a second agent). The two site specific epigenetic modifying agents may be present in the same composition, e.g., pharmaceutical composition, e.g., pharmaceutical composition comprising an LNP, or in separate compositions, e.g., pharmaceutical composition, e.g., pharmaceutical composition comprising an LNP. The subject may be administered the first site specific epigenetic modifying agent at one time and administered the second site specific epigenetic modifying agent at a second time, or the subject may be administered both agents at the same time.

In some embodiments, the methods of the invention include contacting a cell with three site-specific epigenetic modifying agent (a first, a second, and a third agent). The three site specific epigenetic modifying agents may be present in the same composition, e.g., pharmaceutical composition, e.g., pharmaceutical composition comprising an LNP, or in separate compositions, e.g., pharmaceutical composition, e.g., pharmaceutical composition comprising an LNP. The cell may be contacted with the first site specific epigenetic modifying agent at one time and contacted with the second site specific epigenetic modifying agent at a second time and contacted with the third site specific epigenetic modifying agent at a third time. In some embodiments, the cell may be contacted with two at the same time and a third agent at a second time. In some embodiments, the cell may be contacted with three agents at the same time.

In addition, the present invention provides methods for preventing at least one symptom in a subject that would benefit from a reduction of CXCL9, CXCL10, and/or CXCL11 gene expression, such as a subject having a disease or disorder associated with CXCL9, CXCL10, and/or CXCL11, e.g., liver disease, by administering to the subject an agent or a composition, or cells of the invention in a prophylactically effective amount.

“Therapeutically effective amount,” as used herein, is intended to include the amount of an agent or composition or cells that, when administered to a patient for treating a subject having a disease or disorder associated with CXCL9, CXCL10, and/or CXCL11, e.g., liver disease, is sufficient to effect treatment of the disease (e.g., by diminishing, ameliorating, or maintaining the existing disease or one or more symptoms of disease or its related comorbidities). The “therapeutically effective amount” may vary depending on the agent or composition or cells, how it is administered, the disease and its severity and the history, age, weight, family history, genetic makeup, stage of pathological processes mediated by CXCL9, CXCL10, and/or CXCL11 gene expression, the types of preceding or concomitant treatments, if any, and other individual characteristics of the patient to be treated.

“Prophylactically effective amount,” as used herein, is intended to include the amount of an agent or composition or cells that, when administered to a subject who does not yet experience or display symptoms of a disease or disorder associated with CXCL9, CXCL10, and/or CXCL11, e.g., liver disease, but who may be predisposed to a disease or disorder associated with CXCL9, CXCL10, and/or CXCL11, e.g., liver disease, is sufficient to prevent or delay the development or progression of the disease or one or more symptoms of the disease for a clinically significant period of time. The “prophylactically effective amount” may vary depending on the agent or composition, how it is administered, the degree of risk of disease, and the history, age, weight, family history, genetic makeup, the types of preceding or concomitant treatments, if any, and other individual characteristics of the patient to be treated.

As used herein, “prevention” or “preventing,” when used in reference to a disease, disorder or condition thereof, that would benefit from a reduction in expression of a CXCL9, CXCL10, and/or CXCL11 gene or production of CXCL9, CXCL10, and/or CXCL11 protein, refers to a reduction in the likelihood that a subject will develop a symptom associated with such a disease, disorder, or condition, e.g., a sign or symptom of CXCL9, CXCL10, and/or CXCL11 gene expression or CXCL9, CXCL10, and/or CXCL11 activity.

A “therapeutically-effective amount” or “prophylactically effective amount” also includes an amount of an agent or composition that produces some desired local or systemic effect at a reasonable benefit/risk ratio applicable to any treatment. Agents and compositions employed in the methods of the present invention may be administered in a sufficient amount to produce a reasonable benefit/risk ratio applicable to such treatment. In some embodiments, a therapeutically effective amount or prophylactically effect amount is administered in a single dose; in some embodiments, multiple unit doses are required to deliver a therapeutically or prophylactically effective amount.

As used herein, the phrase “symptoms are reduced” may be used when one or more symptoms of a particular disease, disorder or condition is reduced in magnitude (e.g., intensity, severity, etc.) and/or frequency. In some embodiments, a delay in the onset of a particular symptom is considered one form of reducing the frequency of that symptom.

When the subject to be treated is a mammal such as a human, the composition or cells can be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection.

As used herein, the term “a disease or disorder associated with CXCL9, CXCL10, and/or CXCL11,” includes a disease or disorder or condition that would benefit from a reduction in CXCL9, CXCL10, and/or CXCL11 gene expression, replication, or protein activity, such as a disease or disorder that is associated with IFN gamma signaling. Non-limiting examples of a disease or disorder associated with CXCL9, CXCL10, and/or CXCL11 include acute and chronic liver disease, alagille syndrome, autoimmune hepatitis, biliary atresia, cirrhosis, endoscopic retrograde cholangiopancreatography (ERCP), hemochromatosis, hepatitis A, hepatitis B, alopecia areata, multiple sclerosis, chronic pain, and Human T-lymphotropic virus type 1 (HTLV-1)-associated myelopathy/tropical spastic paraparesis (HAM/TSP).

Administration of the agents or compositions or cells of the invention according to the methods of the invention may result in a reduction of the severity, signs, symptoms, or markers of a disease or disorder associated with CXCL9, CXCL10, and/or CXCL11 in a patient with a disease or disorder associated with CXCL9, CXCL10, and/or CXCL11. By “reduction” in this context is meant a statistically significant decrease in such level. The reduction (absolute reduction or reduction of the difference between the elevated level in the subject and a normal level) can be, for example, at least about 20%, 25%, 30%, 35%, 40%, 45%, 50%, 55%, 60%, 65%, 70%, 75%, 80%, 85%, 90%, or 95%, or to below the level of detection of the assay used.

Administration of the agents or compositions or cells according to the methods of the invention may stably or transiently reduce expression of a target gene. In some embodiments, a reduction of expression persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some other embodiments, a reduction of expression persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

The agents or compositions or cells may be administered once to the subject or, alternatively, multiple administrations may be performed over a period of time. For example, two, three, four, five, or more administrations may be given to the subject during one treatment or over a period of time. In some embodiments, six, eight, ten, 12, 15 or 20 or more administrations may be given to the subject during one treatment or over a period of time as a treatment regimen.

In some embodiments, administrations may be given as needed, e.g., for as long as symptoms associated with the disease, disorder or condition persist. In some embodiments, repeated administrations may be indicated for the remainder of the subject's life. Treatment periods may vary and could be, e.g., one day, two days, three days, one week, two weeks, one month, two months, three months, six months, a year, or longer.

Efficacy of treatment or prevention of disease can be assessed, for example by measuring disease progression, disease remission, symptom severity, reduction in pain, quality of life, dose of a medication required to sustain a treatment effect, level of a disease marker, or any other measurable parameter appropriate for a given disease being treated or targeted for prevention. It is well within the ability of one skilled in the art to monitor efficacy of treatment or prevention by measuring any one of such parameters, or any combination of parameters. As discussed herein, the specific parameters to be measured depend on the disease or disorder associated with CXCL9, CXCL10, and/or CXCL11 that the subject is suffering from.

Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an agent or composition, “effective against” a disease or disorder associated with CXCL9, CXCL10, and/or CXCL11 indicates that administration in a clinically appropriate manner results in a beneficial effect for at least a statistically significant fraction of patients, such as an improvement of symptoms, a cure, a reduction in disease, extension of life, improvement in quality of life, or other effect generally recognized as positive by medical doctors familiar with treating a disease or disorder associated with CXCL9, CXCL10, and/or CXCL11.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given agent or composition can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using an agent or composition as described herein.

As used herein, the terms “treating” or “treatment” refer to a beneficial or desired result including, but not limited to, alleviation or amelioration of one or more signs or symptoms associated with CXCL9, CXCL10, and/or CXCL11 gene expression or CXCL9, CXCL10, and/or CXCL11 protein production. “Treatment” can also mean prolonging survival as compared to expected survival in the absence of treatment.

### Combination Methods

The present invention further provides combination methods to reduce inflammation in the liver. In some embodiments, treatment with a site-specific epigenetic modifying agent suppressing expression of CXCL9, CXCL10, and/or CXCL11 can be combined with other treatments for a disease or disorder associated with CXCL9, CXCL10, and/or CXCL11.

The present invention is next described by means of the following examples. However, the use of these and other examples anywhere in the specification is illustrative only, and in no way limits the scope and meaning of the invention or of any exemplified form. The invention is not limited to any particular preferred embodiments described herein. Many modifications and variations of the invention may be apparent to those skilled in the art and can be made without departing from its spirit and scope. The contents of all references, patents and published patent applications cited throughout this application, including the figures and informal sequence listing, are incorporated herein by reference.

### EXAMPLES

### Example 1. Mouse Surrogate OECs Downregulate CXCL9/10/11

The objective of this study is to determine the effect of murine OEC treatment on CXCL9, CXCL10, and CXCL11 mRNA expression in interferon-γ (IFNG) stimulated murine hepatic cells (AML-12). Induction of chemokines in response to liver injury results in the recruitment of T-cells to the site of injury and the development of chronic inflammation. Inhibition of the CXCL9/10/11 chemokines represents a mechanism for preventing the pathologic T-cell migration in liver disease. To assess this, all test articles are formulated in MC3 lipids for cellular delivery. Lipid nanoparticles (LNP) were formulated using the NanoAssemblr® Spark™ according to the steps outlined in the spark formulations protocol. AML-12 (ATCC) cells were plated in DMEM: F-12 media on 96-well plates at a density of 10,000 cells/well and cultured overnight. Cells were treated with 5 ng/ml interferon gamma (R&D Systems) and transfected with varying concentrations of OECs formulated in the MC3 lipid. Cells were collected 24 hours following continuous treatment. RNA was extracted from cells and qRT-PCR was run on Quantstudio 7 using Taqman probes to evaluate CXCL9/10/11 mRNA levels.

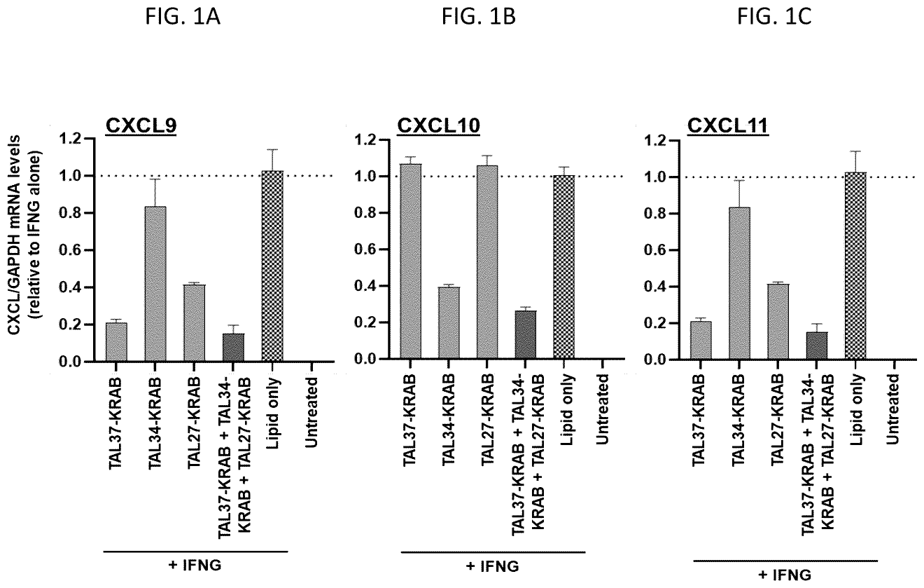

Treatment with TAL-37-KRAB, TAL34-KRAB and TAL27-KRAB down-regulated the mRNA expression levels of CXCL9, CXCL10, and CXCL11 (respectively), that was induced by IFNG treatment in AML-12 cells (FIGS. 1A-1C). This result demonstrates OEC-targeting can impair the IFNG-stimulated increase in chemokine signaling that is involved in the pathophysiology of inflammatory liver disease.

### Example 2. Treatment with Human Monocistronic OECs Reduces CXCL9/10/11 mRNA Levels in Primary Human Hepatocytes

The objective of this study is to determine the effect of human monocistronic OEC treatment on CXCL9, CXCL10, and CXCL11 mRNA expression in IFNG-stimulated primary human hepatocytes. To assess this, all test articles are formulated in MC3 lipids for cellular delivery. Lipid nanoparticles (LNP) were formulated using the NanoAssemblr® Spark™ according to the steps outlined in the spark formulations protocol. Primary human hepatocytes (Lonza) were plated in HBM media on collagen-coated 96-well plates at a density of 50,000 cells per well and cultured overnight. Cells were treated with 5 ng/ml interferon gamma (R&D Systems) and transfected with 2.5 μg/ml OECs formulated in the MC3 lipid. Cells were collected 24 hours following continuous treatment. RNA was extracted from cells and qRT-PCR was run on QuantStudio 7 using Taqman probes to evaluate CXCL9/10/11 mRNA levels.

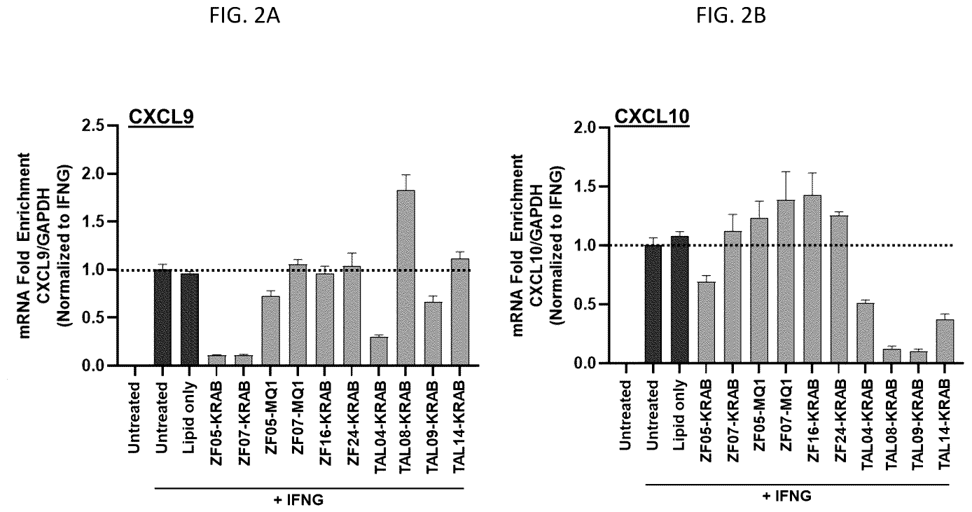

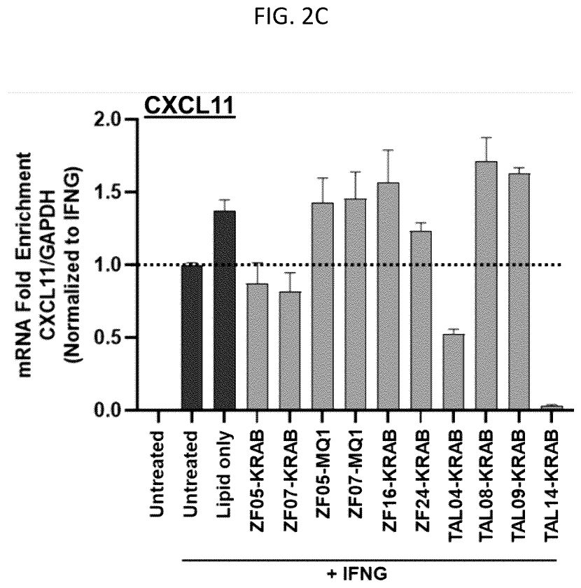

Treatment with human monocistronic OECs down-regulated the mRNA expression levels of CXCL9, CXCL10, and CXCL11, each which was induced by IFNG treatment in primary human hepatocytes (FIGS. 2A-2C). This result demonstrates OEC-targeting can impair the IFNG-stimulated increase in chemokine mRNA levels involved in the pathophysiology of inflammatory liver disease.

### Example 3. Treatment with Human Monocistronic OECs Reduces CXCL9/10/11 Protein Levels in Conditioned Media from Primary Human Hepatocytes

The objective of this study is to determine the effect of human monocistronic OEC treatment on CXCL9, CXCL10, and CXCL11 protein expression in IFNG-stimulated primary human hepatocytes. To assess this, all test articles are formulated in MC3 lipids for cellular delivery. Lipid nanoparticles (LNP) were formulated using the NanoAssemblr® Spark™ according to the steps outlined in the spark formulations protocol. Primary human hepatocytes (Lonza) were plated in HBM media on collagen-coated 96-well plates at a density of 50,000 cells per well and cultured overnight. Cells were treated with 5 ng/ml interferon gamma (R&D Systems) and transfected with 2.5 μg/ml OECs formulated in the MC3 lipid. Conditioned media were collected 24 hours following continuous treatment. Conditioned media was diluted and run on custom Luminex plates to assess CXCL9/10/11 protein levels.

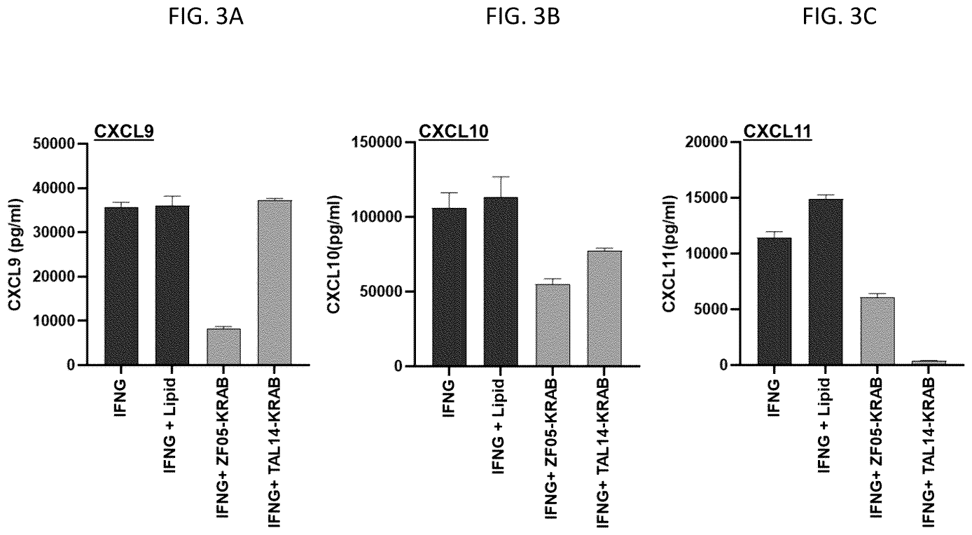

Analysis of conditioned media demonstrated that ZF05-KRAB down-regulated CXCL9, CXCL10 and CXCL11 protein levels whereas TAL14-KRAB downregulated CXCL11 protein levels (FIGS. 3A-3C). This result demonstrates OEC-targeting can impair the IFNG-stimulated increase in chemokine levels that is involved in the pathophysiology of inflammatory liver disease.

### Example 4. Treatment with Single Human Bicistronic OEC Reduces CXCL9/10/11 mRNA Levels in Primary Human Hepatocytes

The objective of this study is to determine the effect of a single human bicistronic OEC ZF05-KRAB_TAL14-KRAB on CXCL9, CXCL10, and CXCL11 mRNA expression in interferon-γ (IFNG) stimulated human hepatic cells (Lonza). Induction of chemokines in response to liver injury results in the recruitment of T-cells to the site of injury and the development of chronic inflammation. Inhibition of the CXCL9/10/11 chemokines represents a mechanism for preventing the pathologic T-cell migration in liver disease. To assess this, all test articles are formulated in MC3 lipids for cellular delivery. Lipid nanoparticles (LNP) were formulated using the NanoAssemblr® Spark™ according to the steps outlined in the spark formulations protocol. Primary Human Hepatocytes cells were plated in Hepatocyte Plating media on collagen-coated 96-well plates at a density of 50,000 cells per well and cultured overnight. Cells were treated with 5 ng/ml interferon gamma (R&D Systems) in HCM media and transfected with varying concentrations of OECs formulated in the MC3 lipid. Cells were lysed 24 hours later in RLT Plus Lysis buffer for mRNA extraction using RNeasy Plus 96 kit. After removal of genomic DNA, the lysate was bound to an RNA column, washed with buffers, and eluted off the column with RNAse free water. The total RNA was converted to cDNA with RT Lunascript. qRT-PCR was run on QuantStudio 7 using Taqman probes to evaluate CXCL9/10/11 mRNA levels.

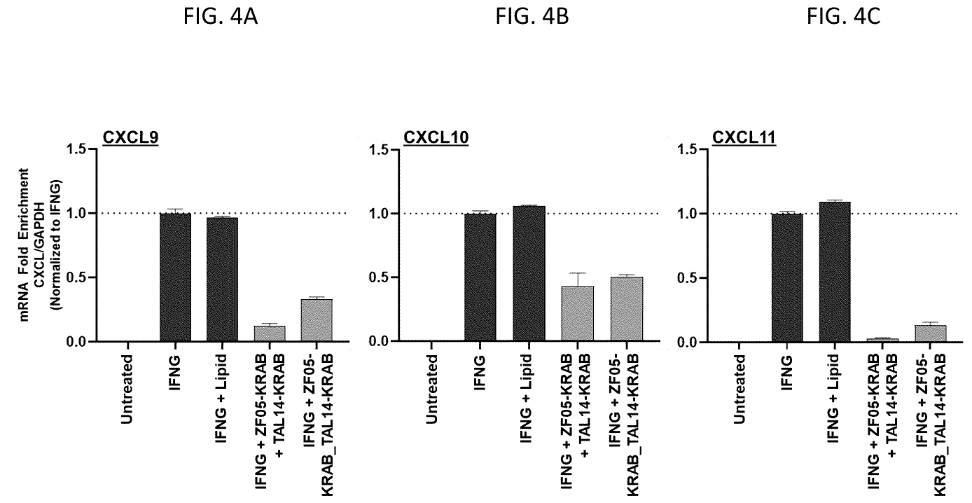

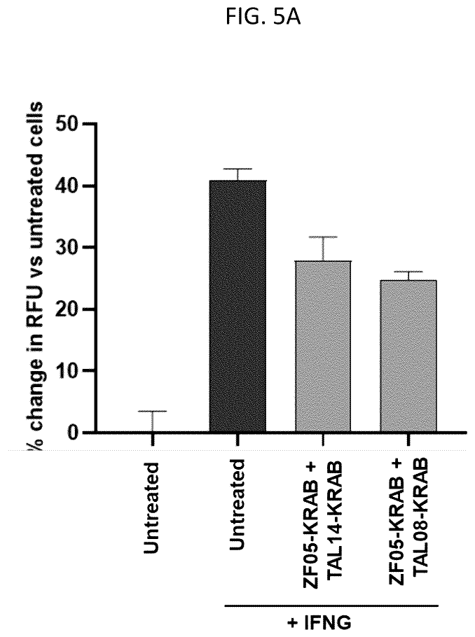

Treatment with the monocistronic combination ZF05-KRAB+TAL14-KRAB and the single bicistronic ZF05-KRAB_TAL14-KRAB down-regulated the mRNA expression levels of CXCL9, CXCL10, and CXCL11, that was induced by IFNG treatment in primary human hepatocytes (FIGS. 4A-4C). This result demonstrates OEC-targeting can impair the IFNG-stimulated increase in chemokine mRNA levels that are involved in the pathophysiology of inflammatory liver disease.

### Example 5. Hepatocyte Treatment with Human Monocistronic OECs Significantly Reduces T Cell Migration

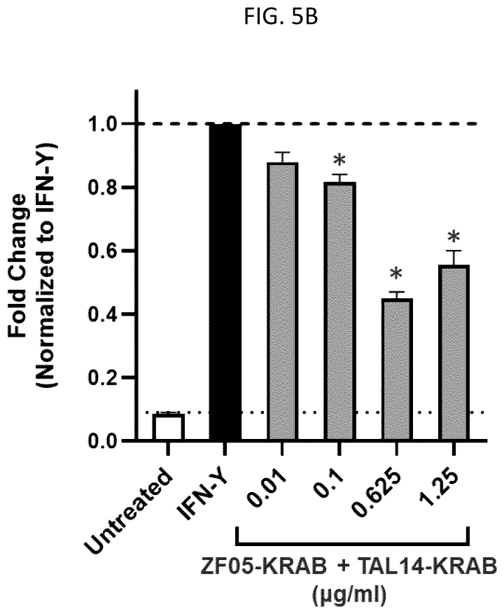

The objective of this study is to determine the effect of human OECs treatment on T cell migration. To assess this, all test articles are formulated in MC3 lipids for cellular delivery. Lipid nanoparticles (LNP) were formulated using the NanoAssemblr® Spark™ according to the steps outlined in the spark formulations protocol. Primary Human Hepatocytes cells were plated in Hepatocyte Plating media on collagen-coated 96-well plates at a density of 50,000 cells per well and cultured overnight. Cells were treated with 5 ng/ml interferon gamma (R&D Systems) in HCM media and transfected with 2.5 ug/ml of OECs formulated in the MC3 lipid. T cell migration was performed using 24-well transwell chamber with 5 μm pores (Cell Biolabs) to analyze the effect of OEC on the reduction of T cell migration. According to the manufacturer's instructions, amounts of 5×10⁵ T cells (STEMCELL Technologies) in 100 μL 0.5% BSA Hepatocyte Culture Medium were seeded in the upper chambers, and 500 μL CM derived from IFNG-treated primary human hepatocytes was added to the lower chambers. The chemotaxis plate was then incubated in a 37° C. cell culture incubator for 2 hours. The migrated T cells were dissociated from the membrane, lysed, and detected using Bright Glo luciferase (Promega). IFNG resulted in a significant increase in T cell migration (˜10 fold) and treatment with combinations of monocistronic human OECs resulted in a significant reduction (up to 55%) in T cell migration at 24 hours (FIG. 5).

TABLE 12
Exemplary Sequences: Epigenetic Modifying Agents

| Name | Nucleic Acid Sequence |
|---|---|
| OEC695 | ATGGCCCCCAAGAAGAAGCGGAAGGTGGGCATCCACGGCGTGCCCG |
| ZF07-KRAB | CCGCCGGCAGCAGCGGATCCCTCGAGCCTGGCGAAAAGCCTTATAAG |
| (SEQ ID NO: 433) | TGCCCAGAATGCGGAAAGAGCTTCTCCGACAAGAAGGATCTGACCCG |
| | CCACCAAAGGACCCACACTGGGGAGAAGCCTTACAAGTGTCCGGAGT |
| | GCGGAAAGTCCTTTTCGCAATCGTCCAGCCTGGTCCGGCACCAGAGG |
| | ACGCACACCGGAGAGAAGCCATACAAATGCCCGGAGTGTGGGAAGT |
| | CGTTCTCCACCACTGGAAACCTCACTGTGCACCAGCGCACGCATACT |
| | GGGGAAAAGCCCTATAAGTGTCCAGAGTGCGGGAAGTCCTTCTCAAC |
| | CTCCGGCGAATTGGTCAGACACCAGCGGACACACACCGGCGAGAAG |
| | CCCTACAAGTGCCCTGAGTGCGGCAAAAGCTTCAGCACTACCGGCAA |
| | CCTGACTGTGCATCAGCGGACCCATACCGGAGAAAAACCCTACAAAT |
| | GTCCCGAATGCGGCAAATCCTTCTCCACCTCCCACTCGCTGACCGAAC |
| | ACCAGAGAACCCATACTGGAGAAAAGCCGTACAAGTGCCCGGAATG |
| | TGGAAAGTCTTTCAGCACCTCCGGAAACCTTGTGCGGCATCAGCGCA |
| | CTCACACCGGCAAAAAGACCTCAGCTTCCGGATCCGGTGGAGGATCA |
| | GGCGGCGATGCGAAGTCCCTCACCGCTTGGTCACGGACCCTAGTGAC |
| | CTTCAAAGACGTGTTCGTGGATTTCACCAGGGAAGAATGGAAGCTCC |
| | TGGACACCGCCCAGCAAATACTGTACCGGAACGTCATGCTGGAGAAC |
| | TATAAGAACCTGGTCAGCTTGGGCTACCAGCTCACCAAGCCGGACGT |
| | GATCCTGCGGCTGGAGAAGGGAGAGGAGCCTTGGCTCGTGGAAAGG |
| | GAAATTCATCAGGAGACTCACCCTGACAGCGAAACCGCCTTCGAGAT |
| | TAAGTCCTCCGTGTCCGGAGGAAAGCGGCCCGCCGCCACCAAGAAGG |
| | CCGGCCAGGCCAAGAAGAAGAAGGGCAGCTACCCCTACGACGTGCC |
| | CGACTACGCCTGA |
| OEC693 | ATGGCCCCCAAGAAGAAGCGGAAGGTGGGCATCCACGGCGTGCCCG |
| TAL27-KRAB | CCGCCGGCAGCAGCGGATCCCACATGGTGGACCTCCGCACGCTGGGA |
| (SEQ ID NO: 434) | TACTCGCAACAGCAGCAGGAGAAGATTAAGCCTAAGGTCCGGTCCAC |
| | CGTGGCTCAGCACCATGAGGCGCTAGTGGGCCACGGATTCACCCACG |
| | CGCATATTGTCGCCCTCTCGCAACATCCCGCCGCACTGGGAACCGTCG |
| | CCGTGAAGTACCAGGACATGATTGCCGCCCTGCCTGAAGCTACCCAT |
| | GAAGCCATTGTGGGCGTGGGAAAGAGAGGAGCAGGGGCCAGAGCCT |
| | TGGAGGCCCTGCTCACGGTAGCCGGAGAGCTCAGAGGCCCACCACTG |
| | CAGCTTGACACCGGCCAACTTCTGAAGATCGCCAAGCGGGGAGGAGT |
| | GACCGCCGTGGAGGCAGTGCATGCCTGGCGCAACGCCTTGACTGGAG |
| | CCCCGCTGAACCTGACTCCGGAGCAAGTGGTGGCTATCGCGTCCAAC |
| | ATCGGAGGAAAACAGGCCCTGGAAACGGTGCAAAGACTGCTGCCGG |
| | TCCTCTGTCAAGCGCACGGACTCACTCCCGAGCAAGTIGTGGCTATCG |
| | CTTCCAACATCGGGGGGAAACAGGCTCTGGAAACTGTCCAAAGGCTG |
| | TTACCTGTGCTGTGCCAAGCACACGGACTGACCCCAGAGCAAGTGGT |
| | CGCCATAGCCTCCAACAACGGGGGGAAGCAAGCCCTGGAGACTGTGC |
| | AGCGGTTGCTCCCCGTCCTGTGCCAGGCCCACGGATTGACTCCCGAA |
| | CAAGTGGTGGCCATTGCGTCAAACATCGGGGGCAAGCAAGCGTTGGA |
| | AACAGTCCAACGGTTGCTTCCCGTCCTTTGCCAGGCCCACGGTCTGAC |
| | CCCGGAGCAAGTCGTCGCCATTGCTAGTAACGGGGGAGGAAAGCAA |
| | GCTCTTGAAACCGTCCAGCGGCTTCTTCCGGTGCTCTGCCAAGCACAC |
| | GGCCTCACACCCGAGCAAGTAGTGGCCATTGCCTCAAACATTGGGGG |
| | AAAGCAGGCATTGGAAACTGTGCAACGGCTGCTTCCTGTCTTGTGCC |
| | AGGCGCATGGGCTGACTCCAGAACAGGTGGTCGCTATCGCGAGTAAC |
| | AACGGTGGCAAACAGGCACTGGAAACCGTGCAGAGGCTCTTGCCTGT |
| | CCTGTGTCAGGCACACGGACTGACGCCAGAACAGGTGGTCGCTATTG |
| | CGAGCAATATCGGCGGAAAGCAGGCGTTAGAAACTGTGCAGAGACT |
| | GCTCCCAGTGCTATGCCAGGCTCATGGGTTGACTCCTGAACAAGTGG |
| | TGGCTATCGCCTCAAACGGTGGCGGTAAACAGGCGCTTGAGACAGTG |
| | CAAAGGCTCCTTCCCGTGTTGTGCCAAGCGCATGGCCTGACCCCTGA |
| | ACAGGTCGTGGCTATCGCGTCAAACAACGGAGGCAAGCAGGCCCTAG |
| | AAACGGTACAACGGCTGCTGCCCGTACTGTGCCAGGCCCACGGACTG |
| | ACCCCAGAACAGGTGGTCGCAATTGCCTCCAACATTGGCGGGAAGCA |
| | GGCGCTAGAAACCGTGCAGCGGCTTCTGCCCGTCCTCTGCCAGGCCC |
| | ACGGTCTGACTCCTGAGCAAGTCGTCGCAATCGCCAGCAACATCGGC |
| | GGAAAACAAGCCCTCGAAACCGTCCAGCGACTGCTGCCTGTCCTATG |
| | CCAGGCCCACGGGTTGACCCCGGAACAGGTCGTCGCCATTGCTTCCA |
| | ATAATGGAGGCAAACAGGCACTCGAAACGGTGCAGAGACTTCTGCCG |
| | GTGCTGTGTCAAGCTCACGGCTTGACCCCCGAGCAAGTGGTGGCAAT |
| | CGCTTCACGACACGGTGGAAAACAGGCCCTGGAGACAGTCCAGAGA |
| | CTGCTCCCCGTGCTTTGCCAAGCTCACGGACTTACTCCGGAACAAGTC |
| | GTGGCGATTGCGTCCAACGGTGGTGGAAAGCAGGCCTTGGAAACGGT |
| | CCAGAGACTGTTGCCGGTCTTGTGCCAGGCGCATGGTCTGACCCCCG |
| | AACAAGTCGTGGCAATTGCATCCCATGACGGCGGGAAACAGGCTTTG |
| | GAGACTGTCCAGCGCCTGTTGCCAGTCCTTTGTCAAGCCCACGGGTTG |
| | ACACCCGAACAGGTCGTCGCAATCGCCTCGAACATCGGTGGCAAGCA |
| | GGCCCTTGAAACTGTCCAACGCCTCCTCCCGGTGTTGTGTCAGGCTCA |
| | CGGCCTGACTCCAGAACAAGTCGTGGCCATAGCGTCACACGACGGTG |
| | GACGGCCAGCCCTGGAATCCATCGTGGCGCAGCTGTCCAGACCCGAC |
| | CCAGCACTGGCGGCCCTCACTAACGATCACCTTGTGGCCCTGGCCTGT |
| | CTGGGAGGTAGACCAGCCCTCGACGCTGTGAAGAAGGGACTGCCGCA |
| | TGCACCAGCCCTGATCAAGCGAACCAACCGGCGCATTCCCGAGAGAA |
| | CTTCACACAGAGTGGCTAGCGGCAGCGGCGGCGGCAGCGGCGGCGA |
| | CGCCAAGAGCCTGACCGCCTGGAGCCGGACCCTGGTGACCTTCAAGG |
| | ACGTGTTCGTGGACTTCACCCGGGAGGAGTGGAAGCTGCTGGACACC |
| | GCCCAGCAGATCCTGTACCGGAACGTGATGCTGGAGAACTACAAGAA |
| | CCTGGTGAGCCTGGGCTACCAGCTGACCAAGCCCGACGTGATCCTGC |
| | GGCTGGAGAAGGGCGAGGAGCCCTGGCTGGTGGAGCGGGAGATCCA |
| | CCAGGAAACCCACCCCGACAGCGAAACCGCCTTCGAGATCAAGAGC |
| | AGCGTGAGCGGCGGCAAGCGGCCCGCCGCCACCAAGAAGGCCGGCC |
| | AGGCCAAGAAGAAGAAGGGCAGCTACCCCTACGACGTGCCCGACTA |
| | CGCCTGA |
| OEC151 | ATGGCCCCCAAGAAGAAGCGGAAGGTGGGCATCCACGGCGTGCCCG |
| ZF24-KRAB | CCGCCGGCAGCAGCGGATCCCTGGAACCCGGAGAGAAGCCCTATAA |
| (SEQ ID NO: 435) | GTGTCCGGAATGCGGAAAGAGTTTCAGCTCGAAGAAGCACCTCGCCG |
| | AGCACCAACGGACCCACACGGGAGAAAAGCCCTACAAGTGCCCAGA |
| | GTGCGGAAAGTCGTTCAGCCAAAACTCCACCCTGACCGAACACCAGC |
| | GGACTCATACCGGGGAGAAACCTTACAAGTGTCCAGAATGCGGGAA |
| | GTCGTTCTCCCAATCCGGAGACTTGCGCAGACACCAGCGCACTCACA |
| | CCGGCGAAAAACCGTACAAATGCCCGGAGTGTGGAAAGTCATTTTCC |
| | ACCCACCTGGATCTGATCAGGCATCAGCGCACCCACACTGGCGAAAA |
| | GCCTTACAAATGTCCGGAGTGCGGCAAATCCTTCTCTGACCCCGGCA |
| | ACCTCGTGCGCCATCAGAGGACTCACACTGGGGAGAAGCCTTATAAG |
| | TGCCCCGAATGTGGAAAGAGCTTCTCCACCTCCGGCAACCTTACCGA |
| | GCATCAGCGGACACACACCGGGGAAAAGCCGTACAAGTGCCCTGAG |
| | TGTGGCAAATCATTCTCCGAACGGAGCCATCTGCGGGAGCACCAGAG |
| | AACCCACACCGGAAAGAAAACTTCCGCTTCCGGATCCGGTGGAGGAT |
| | CAGGCGGCGATGCGAAGTCCCTCACCGCTTGGTCACGGACCCTAGTG |
| | ACCTTCAAAGACGTGTTCGTGGATTTCACCAGGGAAGAATGGAAGCT |
| | CCTGGACACCGCCCAGCAAATACTGTACCGGAACGTCATGCTGGAGA |
| | ACTATAAGAACCTGGTCAGCTTGGGCTACCAGCTCACCAAGCCGGAC |
| | GTGATCCTGCGGCTGGAGAAGGGAGAGGAGCCTTGGCTCGTGGAAA |
| | GGGAAATTCATCAGGAGACTCACCCTGACAGCGAAACCGCCTTCGAG |
| | ATTAAGTCCTCCGTGTCCGGAGGAAAGCGGCCCGCCGCCACCAAGAA |
| | GGCCGGCCAGGCCAAGAAGAAGAAGGGCAGCTACCCCTACGACGTG |
| | CCCGACTACGCCTG |
| OEC144 | ATGGCCCCCAAGAAGAAGCGGAAGGTGGGCATCCACGGCGTGCCCG |
| ZF16-KRAB | CCGCCGGCAGCAGCGGATCCCTGGAGCCGGGAGAGAAGCCTTATAA |
| (SEQ ID NO: 436) | GTGTCCTGAGTGCGGCAAATCATTCTCCCAACGCGCCCACCTCGAGA |
| | GACACCAGAGAACCCACACCGGGGAAAAGCCGTACAAGTGCCCTGA |
| | GTGTGGGAAGTCCTTTTCCCGGTCCGACCATTTGACCAACCATCAGCG |
| | GACCCACACTGGGGAGAAGCCATACAAATGCCCAGAATGCGGCAAA |
| | AGCTTCTCCCGGAACGACGCACTTACCGAACATCAGCGCACGCACAC |
| | CGGAGAGAAACCCTATAAGTGCCCCGAATGTGGAAAGTCATTCTCCC |
| | GGGCCGACAACCTGACAGAGCACCAGCGGACTCACACTGGAGAAAA |
| | GCCTTACAAGTGTCCCGAATGCGGGAAGTCGTTCAGCTCGAAGAAGG |
| | CTCTGACCGAACACCAGAGGACCCATACCGGCGAAAAGCCCTATAAA |
| | TGCCCGGAGTGCGGAAAGAGCTTCAGCAGGAACGATGCCCTCACCGA |
| | GCACCAACGCACTCATACTGGCGAAAAACCGTACAAATGTCCGGAAT |
| | GCGGAAAGTCCTTCTCGCACAAGAACGCGCTGCAGAATCACCAGCGC |
| | ACTCACACCGGCAAAAAGACCTCTGCTTCCGGATCCGGTGGAGGATC |
| | AGGCGGCGATGCGAAGTCCCTCACCGCTTGGTCACGGACCCTAGTGA |
| | CCTTCAAAGACGTGTTCGTGGATTTCACCAGGGAAGAATGGAAGCTC |
| | CTGGACACCGCCCAGCAAATACTGTACCGGAACGTCATGCTGGAGAA |
| | CTATAAGAACCTGGTCAGCTTGGGCTACCAGCTCACCAAGCCGGACG |
| | TGATCCTGCGGCTGGAGAAGGGAGAGGAGCCTTGGCTCGTGGAAAG |
| | GGAAATTCATCAGGAGACTCACCCTGACAGCGAAACCGCCTTCGAGA |
| | TTAAGTCCTCCGTGTCCGGAGGAAAGCGGCCCGCCGCCACCAAGAAG |
| | GCCGGCCAGGCCAAGAAGAAGAAGGGCAGCTACCCCTACGACGTGC |
| | CCGACTACGCCTG |
| OEC134 | ATGGCCCCCAAGAAGAAGCGGAAGGTGGGCATCCACGGCGTGCCCG |
| ZF07-MQ1 | CCGCCGGCAGCAGCGGATCCCTCGAGCCTGGCGAAAAGCCTTATAAG |
| (SEQ ID NO: 437) | TGCCCAGAATGCGGAAAGAGCTTCTCCGACAAGAAGGATCTGACCCG |
| | CCACCAAAGGACCCACACTGGGGAGAAGCCTTACAAGTGTCCGGAGT |
| | GCGGAAAGTCCTTTTCGCAATCGTCCAGCCTGGTCCGGCACCAGAGG |
| | ACGCACACCGGAGAGAAGCCATACAAATGCCCGGAGTGTGGGAAGT |
| | CGTTCTCCACCACTGGAAACCTCACTGTGCACCAGCGCACGCATACT |
| | GGGGAAAAGCCCTATAAGTGTCCAGAGTGCGGGAAGTCCTTCTCAAC |
| | CTCCGGCGAATTGGTCAGACACCAGCGGACACACACCGGCGAGAAG |
| | CCCTACAAGTGCCCTGAGTGCGGCAAAAGCTTCAGCACTACCGGCAA |
| | CCTGACTGTGCATCAGCGGACCCATACCGGAGAAAAACCCTACAAAT |
| | GTCCCGAATGCGGCAAATCCTTCTCCACCTCCCACTCGCTGACCGAAC |
| | ACCAGAGAACCCATACTGGAGAAAAGCCGTACAAGTGCCCGGAATG |
| | TGGAAAGTCTTTCAGCACCTCCGGAAACCTTGTGCGGCATCAGCGCA |
| | CTCACACCGGCAAAAAGACCTCAGCATCTGGGAGCGGAGGAGGATC |
| | CGGCGGAGCCAGAGACTCCAAGGTCGAGAACAAGACCAAGAAGCTG |
| | CGGGTGTTCGAGGCCTTTGCCGGGATCGGAGCACAGCGGAAGGCCTT |
| | GGAGAAGGTCCGCAAGGACGAATACGAAATCGTGGGCCTTGCCGAG |
| | TGGTACGTGCCAGCCATCGTGATGTACCAGGCTATCCACAACAACTT |
| | CCACACCAAGCTGGAGTACAAGTCCGTGAGCAGAGAAGAGATGATC |
| | GACTACCTCGAAAACAAGACCCTGTCCTGGAACTCCAAGAATCCCGT |
| | GTCGAACGGTTACTGGAAGCGCAAGAAGGATGATGAACTGAAGATT |
| | ATCTACAACGCGATTAAGCTGTCCGAAAAGGAAGGAAACATTTTCGA |
| | CATTCGCGATCTGTACAAGAGAACCCTCAAGAACATTGATCTGCTTA |
| | CCTACTCCTTCCCGTGCCAAGATCTCAGCCAGCAGGGGATTCAGAAG |
| | GGGATGAAGCGGGGTTCAGGGACCAGATCAGGCCTGCTCTGGGAGAT |
| | CGAAAGAGCCCTGGACTCCACCGAGAAGAACGATCTCCCGAAATACC |
| | TTCTGATGGAGAACGTCGGCGCTCTCCTGCACAAGAAGAACGAAGAA |
| | GAACTGAACCAGTGGAAGCAGAAGCTGGAGTCACTCGGATACCAGA |
| | ATTCTATTGAGGTGCTGAACGCGGCCGATTTCGGATCCTCGCAAGCC |
| | AGGCGGAGAGTGTTCATGATTTCGACCCTGAACGAGTTCGTGGAGCT |
| | GCCTAAGGGCGACAAGAAGCCCAAGTCCATCAAGAAGGTCCTGAAC |
| | AAGATTGTCAGCGAAAAGGACATCCTCAACAATCTGCTTAAGTACAA |
| | CCTGACCGAGTTTAAAAAGACCAAAAGCAACATCAACAAGGCCTCCC |
| | TGATCGGATACTCCAAGTTCAATTCCGAAGGCTACGTGTACGACCCG |
| | GAATTCACCGGACCAACCTTGACCGCCTCCGGTGCTAACTCCCGGAT |
| | CAAGATCAAGGACGGATCCAACATCCGGAAGATGAACTCCGACGAG |
| | ACTTTTCTGTACATCGGTTTCGACTCCCAAGACGGAAAGCGCGTGAA |
| | CGAGATAGAGTTCCTCACCGAGAATCAGAAGATCTTTGTGTGCGGCA |
| | ACTCCATTAGCGTGGAGGTGCTGGAGGCCATCATCGATAAGATCGGT |
| | GGCCCTTCCTCCGGTGGAAAGCGCCCAGCGGCTACAAAGAAGGCCGG |
| | ACAGGCCAAGAAGAAGAAGGGTTCGTACCCCTACGACGTGCCTGACT |
| | ATGCCTG |
| OEC129 | ATGGCCCCCAAGAAGAAGCGGAAGGTGGGCATCCACGGCGTGCCCG |
| ZF05-MQ1 | CCGCCGGCAGCAGCGGATCCCTGGAGCCCGGAGAGAAACCGTATAA |
| (SEQ ID NO: 438) | GTGCCCGGAATGTGGAAAGTCCTTCTCCCAATCCGGCCACCTGACTG |
| | AGCATCAGCGCACCCACACTGGCGAAAAGCCGTACAAGTGCCCTGAG |
| | TGCGGGAAGTCGTTTAGCCAGCGGGCGAACTTGCGCGCTCATCAGCG |
| | GACTCATACCGGGGAGAAGCCCTATAAATGCCCGGAGTGCGGAAAG |
| | AGCTTCAGCACCAAGAACTCCCTGACTGAACACCAGCGGACCCACAC |
| | CGGAGAGAAGCCTTACAAGTGTCCTGAATGCGGGAAATCCTTCTCCA |
| | CCTCCGGTTCACTCGTGCGCCACCAGAGAACGCACACCGGTGAAAAG |
| | CCTTATAAGTGTCCAGAGTGCGGCAAAAGCTTCAGCCAGTCCGGCAA |
| | CCTCACCGAGCACCAACGGACTCACACTGGAGAAAAGCCCTACAAAT |
| | GTCCGGAATGCGGCAAATCATTCTCGCAAGCCGGTCACCTTGCCTCG |
| | CACCAGCGCACACACACCGGGGAAAAGCCATACAAATGCCCCGAAT |
| | GCGGAAAGTCGTTCTCTCAATCCGGCGACCTGAGAAGGCATCAGAGG |
| | ACCCATACCGGAAAGAAAACCTCCGCATCTGGGAGCGGAGGAGGAT |
| | CCGGCGGAGCCAGAGACTCCAAGGTCGAGAACAAGACCAAGAAGCT |
| | GCGGGTGTTCGAGGCCTTTGCCGGGATCGGAGCACAGCGGAAGGCCT |
| | TGGAGAAGGTCCGCAAGGACGAATACGAAATCGTGGGCCTTGCCGA |
| | GTGGTACGTGCCAGCCATCGTGATGTACCAGGCTATCCACAACAACT |
| | TCCACACCAAGCTGGAGTACAAGTCCGTGAGCAGAGAAGAGATGATC |
| | GACTACCTCGAAAACAAGACCCTGTCCTGGAACTCCAAGAATCCCGT |
| | GTCGAACGGTTACTGGAAGCGCAAGAAGGATGATGAACTGAAGATT |
| | ATCTACAACGCGATTAAGCTGTCCGAAAAGGAAGGAAACATTTTCGA |
| | CATTCGCGATCTGTACAAGAGAACCCTCAAGAACATTGATCTGCTTA |
| | CCTACTCCTTCCCGTGCCAAGATCTCAGCCAGCAGGGGATTCAGAAG |
| | GGGATGAAGCGGGGTTCAGGGACCAGATCAGGCCTGCTCTGGGAGAT |
| | CGAAAGAGCCCTGGACTCCACCGAGAAGAACGATCTCCCGAAATACC |
| | TTCTGATGGAGAACGTCGGCGCTCTCCTGCACAAGAAGAACGAAGAA |
| | GAACTGAACCAGTGGAAGCAGAAGCTGGAGTCACTCGGATACCAGA |
| | ATTCTATTGAGGTGCTGAACGCGGCCGATTTCGGATCCTCGCAAGCC |
| | AGGCGGAGAGTGTTCATGATTTCGACCCTGAACGAGTTCGTGGAGCT |
| | GCCTAAGGGCGACAAGAAGCCCAAGTCCATCAAGAAGGTCCTGAAC |
| | AAGATTGTCAGCGAAAAGGACATCCTCAACAATCTGCTTAAGTACAA |
| | CCTGACCGAGTTTAAAAAGACCAAAAGCAACATCAACAAGGCCTCCC |
| | TGATCGGATACTCCAAGTTCAATTCCGAAGGCTACGTGTACGACCCG |
| | GAATTCACCGGACCAACCTTGACCGCCTCCGGTGCTAACTCCCGGAT |
| | CAAGATCAAGGACGGATCCAACATCCGGAAGATGAACTCCGACGAG |
| | ACTTTTCTGTACATCGGTTTCGACTCCCAAGACGGAAAGCGCGTGAA |
| | CGAGATAGAGTTCCTCACCGAGAATCAGAAGATCTTTGTGTGCGGCA |
| | ACTCCATTAGCGTGGAGGTGCTGGAGGCCATCATCGATAAGATCGGT |
| | GGCCCTTCCTCCGGTGGAAAGCGCCCAGCGGCTACAAAGAAGGCCGG |
| | ACAGGCCAAGAAGAAGAAGGGTTCGTACCCCTACGACGTGCCTGACT |
| | ATGCCTG |
| OEC126 | ATGGCCCCCAAGAAGAAGCGGAAGGTGGGCATCCACGGCGTGCCCG |
| ZF05-KRAB | CCGCCGGCAGCAGCGGATCCCTGGAGCCCGGAGAGAAACCGTATAA |
| (SEQ ID NO: 439) | GTGCCCGGAATGTGGAAAGTCCTTCTCCCAATCCGGCCACCTGACTG |
| | AGCATCAGCGCACCCACACTGGCGAAAAGCCGTACAAGTGCCCTGAG |
| | TGCGGGAAGTCGTTTAGCCAGCGGGCGAACTTGCGCGCTCATCAGCG |
| | GACTCATACCGGGGAGAAGCCCTATAAATGCCCGGAGTGCGGAAAG |
| | AGCTTCAGCACCAAGAACTCCCTGACTGAACACCAGCGGACCCACAC |
| | CGGAGAGAAGCCTTACAAGTGTCCTGAATGCGGGAAATCCTTCTCCA |
| | CCTCCGGTTCACTCGTGCGCCACCAGAGAACGCACACCGGTGAAAAG |
| | CCTTATAAGTGTCCAGAGTGCGGCAAAAGCTTCAGCCAGTCCGGCAA |
| | CCTCACCGAGCACCAACGGACTCACACTGGAGAAAAGCCCTACAAAT |
| | GTCCGGAATGCGGCAAATCATTCTCGCAAGCCGGTCACCTTGCCTCG |
| | CACCAGCGCACACACACCGGGGAAAAGCCATACAAATGCCCCGAAT |
| | GCGGAAAGTCGTTCTCTCAATCCGGCGACCTGAGAAGGCATCAGAGG |
| | ACCCATACCGGAAAGAAAACCTCCGCTTCCGGATCCGGTGGAGGATC |
| | AGGCGGCGATGCGAAGTCCCTCACCGCTTGGTCACGGACCCTAGTGA |
| | CCTTCAAAGACGTGTTCGTGGATTTCACCAGGGAAGAATGGAAGCTC |
| | CTGGACACCGCCCAGCAAATACTGTACCGGAACGTCATGCTGGAGAA |
| | CTATAAGAACCTGGTCAGCTTGGGCTACCAGCTCACCAAGCCGGACG |
| | TGATCCTGCGGCTGGAGAAGGGAGAGGAGCCTTGGCTCGTGGAAAG |
| | GGAAATTCATCAGGAGACTCACCCTGACAGCGAAACCGCCTTCGAGA |
| | TTAAGTCCTCCGTGTCCGGAGGAAAGCGGCCCGCCGCCACCAAGAAG |
| | GCCGGCCAGGCCAAGAAGAAGAAGGGCAGCTACCCCTACGACGTGC |
| | CCGACTACGCCTG |
| OEC103 | ATGGCCCCCAAGAAGAAGCGGAAGGTGGGCATCCACGGCGTGCCCG |
| TAL14-KRAB | CCGCCGGCAGCAGCGGATCCCACATGGTCGATCTTCGCACCCTCGGG |
| (SEQ ID NO: 440) | TACTCGCAGCAGCAACAGGAAAAGATCAAGCCGAAAGTCAGGTCCA |
| | CCGTCGCCCAGCACCACGAGGCACTGGTCGGCCACGGATTCACCCAC |
| | GCCCACATCGTCGCGCTGTCTCAACATCCAGCCGCCCTGGGAACTGTC |
| | GCCGTCAAGTACCAAGATATGATCGCGGCACTGCCTGAAGCAACCCA |
| | TGAGGCCATCGTGGGTGTGGGAAAGAGAGGAGCAGGGGCTAGAGCC |
| | CTGGAAGCGCTCCTGACCGTGGCGGGAGAGCTTAGAGGCCCTCCCCT |
| | GCAATTGGACACCGGCCAACTTCTGAAGATTGCAAAAAGGGGTGGAG |
| | TGACTGCCGTGGAAGCAGTGCACGCTTGGCGGAACGCACTGACTGGA |
| | GCACCCCTGAATTTGACCCCCGAGCAAGTGGTTGCCATCGCAAGCAA |
| | CATCGGAGGGAAACAAGCACTGGAAACTGTGCAGCGCCTGCTTCCGG |
| | TCCTGTGCCAAGCTCACGGACTGACTCCTGAACAGGTTGTGGCCATC |
| | GCGTCGCATGACGGGGGAAAACAGGCGCTCGAAACCGTCCAAAGAC |
| | TGCTCCCGGTGCTGTGTCAGGCTCACGGTTTGACCCCAGAACAAGTTG |
| | TCGCTATCGCCTCACACGACGGCGGGAAGCAAGCTCTGGAGACTGTG |
| | CAAAGACTCCTTCCTGTGCTGTGCCAAGCTCATGGATTGACCCCGGA |
| | ACAGGTCGTCGCCATCGCTTCCAATGGCGGAGGTAAACAGGCCTTGG |
| | AAACGGTGCAGAGGCTGCTGCCAGTCTTGTGTCAGGCGCACGGGCTG |
| | ACTCCAGAGCAAGTAGTAGCCATCGCCAGCAATGGAGGCGGTAAAC |
| | AAGCGCTCGAGACCGTCCAGCGCTTGTTGCCAGTGTTGTGCCAGGCC |
| | CATGGGCTGACGCCCGAACAGGTGGTGGCCATTGCATCCAACATTGG |
| | GGGGAAACAGGCGTTGGAGACAGTCCAACGGCTTCTGCCCGTCCTTT |
| | GTCAGGCACACGGACTCACTCCTGAACAAGTCGTTGCCATTGCCTCG |
| | AATATCGGGGGTAAGCAAGCCCTCGAGACTGTGCAGAGATTGTTGCC |
| | TGTCCTGTGTCAAGCGCATGGTCTGACTCCCGAACAGGTGGTGGCGA |
| | TTGCTTCGAACAATGGAGGAAAACAAGCATTGGAAACCGTCCAGAGG |
| | CTGTTACCAGTCCTTTGCCAGGCACATGGACTGACCCCGGAACAGGT |
| | CGTCGCAATCGCATCGAACATTGGAGGCAAGCAGGCTCTCGAAACGG |
| | TCCAGAGACTGCTGCCTGTGCTCTGCCAAGCCCACGGCCTCACTCCGG |
| | AGCAAGTCGTGGCAATCGCCTCAAACATCGGTGGAAAGCAGGCCCTT |
| | GAGACTGTCCAGCGACTGCTCCCAGTGCTCTGCCAGGCGCATGGACT |
| | CACACCCGAGCAGGTGGTCGCCATTGCTAGCAACAACGGAGGCAAAC |
| | AAGCCCTGGAGACAGTGCAACGCTTGCTGCCCGTGCTTTGTCAAGCG |
| | CACGGACTTACTCCGGAACAAGTAGTGGCTATTGCGAGTAGGCACGG |
| | CGGTAAGCAAGCCCTGGAGACAGTGCAGCGGCTTTTGCCGGTGCTCT |
| | GTCAGGCCCACGGGCTCACACCGGAACAGGTCGTAGCTATTGCGTCC |
| | CATGACGGCGGAAAGCAAGCCTTAGAAACTGTGCAGCGGTTGCTGCC |
| | TGTTCTGTGCCAGGCTCATGGGCTCACTCCCGAGCAAGTGGTCGCGAT |
| | TGCGAGCAACGGAGGTGGCAAGCAAGCACTAGAAACCGTGCAACGA |
| | CTGCTGCCGGTCTTGTGCCAAGCGCATGGCCTGACACCCGAACAGGT |
| | GGTGGCTATCGCGTCCAACAACGGGGGCAAACAGGCCCTGGAAACA |
| | GTCCAAAGGCTGTTGCCGGTCCTCTGTCAAGCTCACGGACTAACCCCC |
| | GAACAGGTGGTGGCAATAGCTTCGCGGCATGGGGGCAAGCAGGCAC |
| | TCGAAACAGTGCAGAGGTTGCTCCCGGTACTTTGCCAAGCACATGGC |
| | TTGACCCCTGAGCAAGTCGTGGCCATCGCATCCAACGGCGGAGGGAA |
| | GCAAGCGCTTGAAACTGTCCAACGCCTCCTGCCCGTGTTGTGTCAAGC |
| | CCATGGCCTTACGCCTGAACAAGTCGTCGCTATTGCAAGTAACGGAG |
| | GAGGGAGGCCTGCCCTGGAGTCCATTGTCGCCCAACTGTCGCGACCC |
| | GATCCAGCCCTGGCAGCCCTCACTAACGACCATCTCGTGGCGCTGGC |
| | ATGCCTCGGTGGTAGACCAGCTCTGGACGCCGTGAAGAAGGGACTTC |
| | CTCATGCCCCCGCACTGATCAAGCGCACTAACCGGAGGATCCCTGAA |
| | AGAACCTCCCACAGAGTGGCTTCCGGATCCGGTGGAGGATCAGGCGG |
| | CGATGCGAAGTCCCTCACCGCTTGGTCACGGACCCTAGTGACCTTCA |
| | AAGACGTGTTCGTGGATTTCACCAGGGAAGAATGGAAGCTCCTGGAC |
| | ACCGCCCAGCAAATACTGTACCGGAACGTCATGCTGGAGAACTATAA |
| | GAACCTGGTCAGCTTGGGCTACCAGCTCACCAAGCCGGACGTGATCC |
| | TGCGGCTGGAGAAGGGAGAGGAGCCTTGGCTCGTGGAAAGGGAAAT |
| | TCATCAGGAGACTCACCCTGACAGCGAAACCGCCTTCGAGATTAAGT |
| | CCTCCGTGTCCGGAGGAAAGCGGCCCGCCGCCACCAAGAAGGCCGGC |
| | CAGGCCAAGAAGAAGAAGGGCAGCTACCCCTACGACGTGCCCGACT |
| | ACGCCTG |
| OEC099 | ATGGCCCCCAAGAAGAAGCGGAAGGTGGGCATCCACGGCGTGCCCG |
| TAL09-KRAB | CCGCCGGCAGCAGCGGATCCCACATGGTGGACCTCCGGACCCTGGGA |
| (SEQ ID NO: 441) | TACTCCCAGCAGCAGCAGGAAAAGATCAAGCCGAAGGTCCGCTCCAC |
| | TGTGGCTCAACACCACGAAGCCCTTGTGGGACACGGCTTCACTCATG |
| | CACACATTGTGGCATTGTCCCAACACCCTGCCGCTCTGGGAACTGTGG |
| | CAGTGAAGTACCAGGACATGATAGCCGCACTGCCCGAAGCAACACAC |
| | GAGGCCATCGTCGGTGTGGGAAAAAGAGGAGCGGGAGCCAGAGCAC |
| | TCGAAGCGCTCCTGACAGTGGCGGGAGAACTCCGCGGACCACCTCTG |
| | CAGCTGGACACTGGCCAGCTGCTGAAGATTGCAAAACGCGGTGGAGT |
| | CACCGCTGTGGAGGCGGTGCATGCCTGGAGAAATGCCCTGACTGGTG |
| | CCCCCTTGAACCTGACCCCCGAGCAAGTCGTCGCAATTGCTTCGAAC |
| | AACGGTGGTAAACAGGCTCTCGAAACCGTCCAACGGCTGCTCCCAGT |
| | CCTATGCCAAGCCCATGGACTTACGCCCGAACAAGTGGTTGCTATAG |
| | CCTCCAATATCGGTGGGAAGCAGGCTCTGGAGACAGTCCAAAGACTC |
| | CTGCCGGTCCTCTGCCAAGCGCACGGACTCACCCCAGAGCAAGTAGT |
| | GGCCATTGCCTCCCACGATGGTGGCAAGCAGGCCCTTGAAACAGTCC |
| | AGAGACTGTTACCCGTGCTTTGTCAAGCCCACGGGCTCACTCCCGAA |
| | CAGGTGGTGGCGATTGCCTCTAACGGCGGCGGCAAACAGGCGCTCGA |
| | GACTGTCCAGCGGCTTCTACCGGTGTTGTGCCAGGCCCATGGTCTGAC |
| | ACCCGAACAGGTGGTTGCAATTGCCTCCAACGGAGGCGGGAAGCAA |
| | GCCCTCGAAACGGTCCAGCGGTTGTTGCCGGTCCTGTGTCAGGCGCA |
| | CGGGTTGACCCCAGAACAGGTCGTCGCTATTGCCTCAAACATCGGTG |
| | GTAAGCAGGCGCTGGAAACTGTGCAGCGACTCCTGCCTGTGCTCTGT |
| | CAAGCGCACGGGCTTACGCCAGAACAAGTGGTCGCTATCGCGTCCAA |
| | CAACGGCGGCAAGCAAGCTCTGGAAACCGTCCAAAGGCTCTTGCCCG |
| | TCCTATGTCAGGCCCACGGCCTCACCCCTGAACAGGTTGTCGCCATCG |
| | CCAGCCACGACGGCGGTAAGCAAGCCTTGGAAACCGTGCAACGACTG |
| | CTTCCCGTGTTGTGTCAGGCTCATGGCCTCACTCCTGAGCAAGTTGTG |
| | GCCATCGCTTCGAATATTGGGGGCAAACAAGCCCTGGAAACGGTGCA |
| | GCGGTTGCTGCCTGTCTTGTGTCAAGCTCACGGGCTGACTCCTGAACA |
| | AGTCGTGGCCATAGCGTCCAACATTGGGGGAAAGCAGGCGTTGGAAA |
| | CTGTCCAGAGGCTGTTGCCCGTGCTCTGCCAGGCTCACGGCCTGACCC |
| | CTGAACAGGTGGTCGCAATCGCCTCCAACATCGGAGGGAAGCAGGCA |
| | CTGGAAACAGTGCAGCGGCTGCTGCCCGTGCTGTGCCAAGCTCATGG |
| | ACTCACTCCGGAACAGGTCGTTGCAATCGCATCAAACATTGGAGGAA |
| | AGCAGGCCCTCGAGACAGTGCAGAGACTGCTCCCTGTGCTTTGCCAG |
| | GCGCATGGCCTTACCCCGGAACAAGTGGTAGCCATCGCGTCGCATGA |
| | CGGCGGAAAGCAGGCTTTGGAAACCGTGCAAAGGCTCCTGCCCGTCC |
| | TGTGCCAGGCTCACGGTCTGACCCCGGAACAAGTCGTGGCCATCGCT |
| | AGCCATGATGGAGGGAAACAGGCCCTGGAGACTGTACAGAGACTCCT |
| | CCCTGTCCTTTGCCAAGCACACGGACTTACCCCTGAACAGGTCGTGGC |
| | TATTGCATCGAACGGGGGGGGGAAACAAGCGCTCGAAACTGTGCAA |
| | CGCCTCCTGCCAGTGTTGTGCCAGGCACATGGCTTGACTCCCGAGCA |
| | AGTCGTCGCCATTGCATCAAACAACGGAGGAAAACAGGCACTCGAG |
| | ACTGTGCAAAGACTGTTGCCGGTGCTATGCCAGGCACACGGCTTGAC |
| | CCCGGAGCAAGTGGTGGCTATCGCCTCGCACGATGGCGGAAAACAAG |
| | CACTCGAAACTGTTCAGCGGCTACTGCCGGTGCTCTGCCAGGCTCAC |
| | GGACTGACTCCAGAACAGGTGGTCGCAATCGCGTCGAACGGTGGAGG |
| | ACGGCCTGCCCTCGAGAGCATTGTGGCGCAGCTCTCCCGCCCGGATC |
| | CTGCTTTGGCCGCGCTGACCAACGACCACCTGGTGGCCCTCGCCTGTT |
| | TGGGGGGTCGGCCTGCTTTGGACGCCGTCAAGAAGGGCCTGCCTCAT |
| | GCCCCCGCACTCATCAAGCGGACCAATAGACGCATCCCGGAAAGGAC |
| | TTCTCACCGGGTGGCTTCCGGATCCGGTGGAGGATCAGGCGGCGATG |
| | CGAAGTCCCTCACCGCTTGGTCACGGACCCTAGTGACCTTCAAAGAC |
| | GTGTTCGTGGATTTCACCAGGGAAGAATGGAAGCTCCTGGACACCGC |
| | CCAGCAAATACTGTACCGGAACGTCATGCTGGAGAACTATAAGAACC |
| | TGGTCAGCTTGGGCTACCAGCTCACCAAGCCGGACGTGATCCTGCGG |
| | CTGGAGAAGGGAGAGGAGCCTTGGCTCGTGGAAAGGGAAATTCATC |
| | AGGAGACTCACCCTGACAGCGAAACCGCCTTCGAGATTAAGTCCTCC |
| | GTGTCCGGAGGAAAGCGGCCCGCCGCCACCAAGAAGGCCGGCCAGG |
| | CCAAGAAGAAGAAGGGCAGCTACCCCTACGACGTGCCCGACTACGCC |
| | TG |
| OEC095 | ATGGCCCCCAAGAAGAAGCGGAAGGTGGGCATCCACGGCGTGCCCG |
| TAL08-KRAB | CCGCCGGCAGCAGCGGATCCCACATGGTGGACCTCCGGACTCTTGGT |
| (SEQ ID NO: 442) | TACTCCCAACAGCAGCAGGAAAAGATCAAGCCTAAGGTCCGCTCCAC |
| | TGTGGCCCAACACCACGAGGCCCTTGTGGGACATGGATTCACCCACG |
| | CCCACATCGTGGCGCTGTCGCAGCACCCTGCTGCCTTGGGGACCGTC |
| | GCCGTGAAGTACCAGGACATGATCGCCGCCTTACCCGAAGCCACCCA |
| | CGAGGCAATCGTGGGAGTGGGTAAAAGGGGTGCCGGTGCAAGAGCT |
| | CTGGAGGCCCTTCTCACCGTGGCCGGAGAACTTAGAGGGCCTCCACT |
| | CCAGCTGGATACCGGTCAGCTGTTGAAGATTGCTAAGAGAGGAGGGG |
| | TGACCGCCGTGGAAGCCGTGCATGCGTGGCGCAACGCTCTGACTGGA |
| | GCCCCACTGAACTTGACCCCCGAACAGGTGGTCGCCATCGCCTCCAA |
| | TAACGGGGGCAAACAGGCGCTGGAAACCGTGCAGAGATTGCTGCCTG |
| | TCTTATGCCAAGCACACGGACTCACCCCCGAGCAAGTCGTGGCTATC |
| | GCCTCCAGACACGGCGGGAAGCAAGCTCTGGAGACTGTGCAGAGACT |
| | GCTACCCGTGTTGTGTCAAGCCCACGGTCTTACTCCGGAGCAAGTGGT |
| | CGCTATTGCCTCGCATGATGGTGGTAAACAGGCCCTGGAGACAGTGC |
| | AGCGCTTGCTCCCTGTGCTGTGTCAGGCACACGGCCTGACACCGGAA |
| | CAGGTGGTGGCCATCGCTTCGAACATTGGAGGTAAACAGGCATTGGA |
| | GACAGTCCAGAGGCTCCTGCCGGTCCTCTGCCAAGCTCACGGTCTGA |
| | CTCCTGAACAAGTAGTCGCCATTGCATCCCACGATGGCGGTAAGCAG |
| | GCTCTTGAAACTGTCCAGAGACTGTTACCTGTGCTCTGTCAGGCCCAT |
| | GGACTGACGCCTGAACAGGTCGTCGCGATTGCGTCAAACATCGGTGG |
| | AAAGCAAGCGCTTGAAACGGTCCAGCGCCTACTGCCTGTGTTGTGCC |
| | AAGCGCACGGCCTCACACCAGAACAAGTCGTCGCAATCGCAAGCCAT |
| | GATGGCGGAAAGCAGGCACTAGAAACTGTTCAGCGCCTTCTACCCGT |
| | CTTGTGCCAGGCTCATGGACTCACTCCCGAACAAGTGGTGGCTATTGC |
| | GTCCAATGGAGGCGGTAAACAAGCCCTCGAAACCGTCCAGCGGCTTC |
| | TGCCGGTTCTGTGCCAGGCACATGGCTTGACTCCTGAGCAAGTTGTCG |
| | CGATTGCCTCCAACATTGGGGGCAAGCAAGCACTCGAAACAGTGCAG |
| | AGGCTTCTACCAGTGCTCTGCCAGGCCCACGGTTTGACCCCTGAACA |
| | GGTGGTCGCCATCGCCTCCAACAACGGCGGAAAACAAGCACTGGAA |
| | ACTGTGCAAAGACTGCTTCCTGTGCTGTGTCAGGCGCATGGCCTCACC |
| | CCGGAACAAGTGGTGGCCATAGCGTCGCACGACGGAGGCAAGCAGG |
| | CCCTTGAAACGGTGCAGCGACTGTTGCCCGTCCTTTGTCAGGCTCACG |
| | GGTTAACCCCAGAACAGGTCGTGGCAATCGCCTCTCATGACGGGGGG |
| | AAGCAGGCCTTGGAGACTGTCCAAAGGTTGCTGCCAGTGCTGTGCCA |
| | AGCCCACGGGCTCACTCCAGAGCAAGTAGTGGCGATAGCGAGCCACG |
| | ACGGCGGGAAACAGGCTCTGGAAACCGTACAGAGGTTGCTCCCCGTG |
| | CTTTGTCAAGCACATGGTCTGACCCCCGAGCAAGTCGTGGCCATTGCC |
| | AGCCATGACGGAGGGAAACAGGCACTTGAAACCGTCCAAAGGCTGC |
| | TGCCTGTGCTTTGCCAGGCGCATGGGCTTACTCCTGAACAAGTGGTGG |
| | CGATTGCTTCAAATATCGGTGGCAAACAAGCTTTGGAAACAGTCCAG |
| | CGGTTGCTCCCAGTCCTCTGTCAAGCTCACGGGCTGACGCCAGAACA |
| | GGTCGTGGCAATTGCGTCACACGACGGAGGAAAACAGGCGTTGGAA |
| | ACCGTTCAGCGGCTCTTGCCCGTGCTATGCCAGGCCCACGGACTGACT |
| | CCGGAACAGGTGGTTGCCATCGCCAGCAACAACGGAGGGAAGCAGG |
| | CGCTCGAGACTGTACAGCGACTTCTCCCCGTCCTGTGTCAAGCCCATG |
| | GCCTGACGCCGGAACAGGTGGTAGCCATTGCGAGCAACGGAGGAGG |
| | ACGCCCAGCCCTAGAGAGCATCGTGGCCCAACTGTCGCGGCCGGATC |
| | CAGCTCTGGCCGCATTGACCAACGACCACCTGGTCGCTCTGGCTTGTC |
| | TGGGAGGACGACCGGCCCTGGATGCCGTGAAGAAGGGCCTGCCACAT |
| | GCCCCTGCCCTCATTAAGCGGACTAACAGAAGGATCCCCGAGAGGAC |
| | CTCACACAGAGTCGCTTCCGGATCCGGTGGAGGATCAGGCGGCGATG |
| | CGAAGTCCCTCACCGCTTGGTCACGGACCCTAGTGACCTTCAAAGAC |
| | GTGTTCGTGGATTTCACCAGGGAAGAATGGAAGCTCCTGGACACCGC |
| | CCAGCAAATACTGTACCGGAACGTCATGCTGGAGAACTATAAGAACC |
| | TGGTCAGCTTGGGCTACCAGCTCACCAAGCCGGACGTGATCCTGCGG |
| | CTGGAGAAGGGAGAGGAGCCTTGGCTCGTGGAAAGGGAAATTCATC |
| | AGGAGACTCACCCTGACAGCGAAACCGCCTTCGAGATTAAGTCCTCC |
| | GTGTCCGGAGGAAAGCGGCCCGCCGCCACCAAGAAGGCCGGCCAGG |
| | CCAAGAAGAAGAAGGGCAGCTACCCCTACGACGTGCCCGACTACGCC |
| | TG |
| OEC087 | ATGGCCCCCAAGAAGAAGCGGAAGGTGGGCATCCACGGCGTGCCCG |
| TAL04-KRAB | CCGCCGGCAGCAGCGGATCCCACATGGTGGACCTGAGAACCCTGGGA |
| (SEQ ID NO: 443) | TACTCGCAGCAGCAGCAGGAAAAGATCAAGCCCAAAGTGCGGTCCA |
| | CTGTGGCCCAGCACCATGAGGCCCTCGTCGGACACGGATTCACCCAC |
| | GCGCACATCGTGGCTCTGTCACAGCATCCCGCCGCCTTGGGAACCGT |
| | GGCTGTGAAGTACCAGGACATGATTGCTGCACTTCCTGAGGCGACCC |
| | ACGAAGCCATTGTGGGAGTCGGAAAACGGGGTGCAGGCGCTAGAGC |
| | CCTGGAGGCCCTGTTAACGGTGGCAGGCGAACTGCGGGGACCACCAT |
| | TGCAGCTCGATACCGGCCAGCTGCTGAAGATTGCCAAGAGGGGCGGA |
| | GTCACAGCGGTGGAGGCGGTCCATGCCTGGCGCAACGCTCTTACTGG |
| | GGCTCCACTGAACCTGACCCCTGAACAGGTAGTGGCCATCGCATCGA |
| | ACAATGGCGGTAAGCAGGCTCTCGAAACCGTCCAAAGGCTCCTTCCG |
| | GTGTTATGCCAGGCTCATGGGCTGACGCCTGAACAAGTGGTCGCAAT |
| | CGCATCGCATGACGGGGGGAAACAGGCATTAGAAACCGTGCAACGC |
| | CTCCTTCCTGTCCTTTGTCAAGCACATGGATTGACTCCTGAGCAAGTT |
| | GTCGCGATTGCCTCCAACATTGGCGGCAAACAGGCACTGGAAACCGT |
| | TCAACGGCTCCTCCCCGTACTGTGCCAGGCACACGGCCTGACCCCCG |
| | AACAAGTCGTCGCCATTGCCTCGAACATCGGAGGCAAGCAGGCGCTT |
| | GAAACCGTGCAGCGCTTGCTGCCTGTGCTCTGCCAAGCGCACGGCCT |
| | AACCCCTGAACAGGTGGTAGCAATTGCTTCCCACGACGGTGGGAAGC |
| | AGGCCCTCGAGACAGTGCAAAGACTGTTGCCGGTCCTCTGCCAGGCC |
| | CACGGACTTACCCCCGAGCAAGTCGTGGCTATTGCGTCCAATAATGG |
| | CGGAAAACAGGCACTTGAGACAGTCCAGAGGCTGCTGCCTGTCTTAT |
| | GCCAAGCCCACGGTCTTACTCCAGAACAGGTGGTCGCTATCGCATCA |
| | AACATTGGAGGAAAACAGGCGCTGGAAACTGTCCAACGCCTGTTGCC |
| | CGTGCTCTGTCAGGCTCACGGGCTGACTCCCGAGCAAGTGGTGGCAA |
| | TCGCCTCCAACGGGGGAGGAAAGCAGGCCTTGGAAACCGTACAGCG |
| | GCTGTTGCCTGTGTTGTGCCAAGCTCACGGTCTGACGCCAGAGCAAG |
| | TGGTGGCCATTGCTTCGCACGACGGCGGGAAGCAGGCACTCGAAACG |
| | GTGCAGAGGTTGCTTCCCGTCTTGTGCCAGGCGCACGGACTCACCCC |
| | AGAACAAGTGGTCGCAATTGCTAGCAACATTGGTGGCAAACAAGCGC |
| | TCGAAACTGTACAACGGCTGCTCCCGGTGCTTTGCCAAGCCCACGGG |
| | CTTACCCCAGAGCAAGTGGTGGCCATTGCGAGCAACATCGGGGGCAA |
| | GCAAGCGCTTGAGACTGTACAAAGACTGCTGCCCGTGCTGTGCCAAG |
| | CCCACGGCTTAACCCCGGAACAGGTCGTGGCCATCGCCTCGAATGGA |
| | GGTGGAAAGCAAGCCCTGGAGACTGTGCAGCGGTTGCTGCCCGTTCT |
| | GTGTCAAGCTCACGGACTGACACCGGAACAGGTGGTGGCTATCGCAA |
| | GCAACAACGGCGGAAAGCAAGCCCTGGAGACTGTGCAGCGGCTGCT |
| | GCCAGTGCTGTGTCAGGCGCATGGACTGACCCCCGAACAAGTCGTAG |
| | CCATAGCCAGCCGCCACGGAGGGAAGCAGGCCCTGGAAACAGTCCA |
| | GCGCCTCTTGCCCGTGTTGTGTCAAGCGCATGGCCTCACCCCTGAACA |
| | GGTCGTTGCCATTGCGTCCAGACATGGAGGGAAGCAAGCACTAGAAA |
| | CAGTGCAGAGACTGCTACCCGTCCTGTGCCAGGCCCATGGACTCACG |
| | CCGGAGCAAGTGGTGGCGATAGCATCGAACATTGGTGGGAAACAAG |
| | CCCTTGAAACTGTCCAGAGACTGTTACCTGTGCTTTGTCAGGCACATG |
| | GGCTCACACCAGAACAGGTGGTCGCCATCGCTAGCAACGGAGGGGG |
| | AAAACAGGCTTTGGAGACTGTCCAGCGGCTCCTACCGGTGCTCTGCC |
| | AAGCACACGGATTGACGCCCGAACAGGTTGTGGCGATTGCGTCCAAC |
| | GGTGGAGGACGGCCTGCCCTGGAGAGCATTGTGGCTCAGCTCTCCAG |
| | ACCTGACCCAGCCCTCGCAGCTCTGACTAACGATCATCTGGTGGCGCT |
| | GGCCTGTTTGGGGGGACGCCCTGCTTTGGACGCCGTGAAGAAGGGTC |
| | TGCCACATGCCCCCGCACTGATCAAGCGCACCAACCGGCGAATCCCT |
| | GAGCGCACTTCCCATCGAGTGGCTTCCGGATCCGGTGGAGGATCAGG |
| | CGGCGATGCGAAGTCCCTCACCGCTTGGTCACGGACCCTAGTGACCT |
| | TCAAAGACGTGTTCGTGGATTTCACCAGGGAAGAATGGAAGCTCCTG |
| | GACACCGCCCAGCAAATACTGTACCGGAACGTCATGCTGGAGAACTA |
| | TAAGAACCTGGTCAGCTTGGGCTACCAGCTCACCAAGCCGGACGTGA |
| | TCCTGCGGCTGGAGAAGGGAGAGGAGCCTTGGCTCGTGGAAAGGGA |
| | AATTCATCAGGAGACTCACCCTGACAGCGAAACCGCCTTCGAGATTA |
| | AGTCCTCCGTGTCCGGAGGAAAGCGGCCCGCCGCCACCAAGAAGGCC |
| | GGCCAGGCCAAGAAGAAGAAGGGCAGCTACCCCTACGACGTGCCCG |
| | ACTACGCCTG |
| OEC056 | ATGGCCCCAAAGAAGAAGCGGAAAGTCGGAGGTTCAGGCGGCTCGC |
| TAL37-KRAB | ACATGGTGGACCTGAGAACTCTGGGTTACTCCCAGCAACAGCAAGAA |
| (SEQ ID NO: 444) | AAAATCAAGCCAAAAGTGCGCAGCACTGTCGCGCAGCACCACGAGG |
| | CCCTTGTGGGGCACGGGTTCACCCACGCTCACATCGTGGCCCTGAGC |
| | CAGCATCCGGCAGCCCTGGGAACCGTCGCAGTGAAGTACCAAGACAT |
| | GATCGCCGCCTTGCCGGAAGCCACTCACGAAGCAATTGTGGGAGTGG |
| | GGAAGAGAGGAGCCGGCGCAAGAGCCCTCGAAGCGCTTCTGACTGT |
| | GGCCGGAGAATTGAGAGGCCCCCCACTGCAGCTGGATACCGGACAAC |
| | TGCTGAAGATCGCCAAACGGGGTGGAGTGACCGCCGTGGAAGCGGT |
| | GCACGCGTGGAGAAACGCGCTGACTGGAGCCCCGCTCAACCTGACCC |
| | CTGAACAGGTCGTGGCTATTGCCTCAAACAACGGCGGGAAGCAAGCG |
| | TTGGAGACTGTACAGCGCCTGCTGCCAGTGTTGTGTCAAGCCCACGG |
| | GCTGACTCCCGAGCAAGTGGTGGCCATAGCCAGCAATATCGGAGGTA |
| | AACAGGCTCTCGAGACTGTCCAACGCCTGCTCCCAGTGCTTTGTCAGG |
| | CTCACGGCCTCACTCCTGAACAGGTCGTAGCCATTGCCAGCAACGGT |
| | GGCGGCAAACAAGCCTTAGAGACTGTGCAACGGCTCTTGCCGGTGCT |
| | CTGTCAGGCCCACGGACTGACCCCAGAACAAGTAGTCGCTATCGCGT |
| | CAAACATCGGCGGCAAGCAGGCTTTGGAAACCGTCCAGCGGCTTCTG |
| | CCGGTGCTGTGCCAGGCGCATGGGCTCACCCCCGAACAGGTGGTCGC |
| | AATCGCCTCGAACGGCGGTGGTAAACAAGCCCTCGAAACTGTCCAGC |
| | GCCTCTTGCCCGTCCTGTGTCAGGCCCATGGCCTGACTCCAGAACAGG |
| | TGGTGGCAATAGCGTCCCATGACGGGGGAAAGCAAGCCCTTGAGACT |
| | GTGCAACGGTTGCTCCCCGTGTTATGTCAGGCACATGGTCTGACCCCC |
| | GAACAGGTGGTCGCAATTGCGTCGAACGGCGGAGGAAAGCAAGCTC |
| | TGGAAACGGTCCAACGGCTGCTCCCTGTGCTGTGCCAGGCGCACGGA |
| | CTTACCCCAGAACAAGTGGTCGCCATTGCGTCCAACATCGGAGGGAA |
| | GCAGGCGCTTGAAACCGTGCAGAGGTTGCTGCCGGTGTTATGCCAAG |
| | CACATGGACTAACCCCGGAGCAAGTGGTGGCCATTGCCTCGAATATT |
| | GGGGGGAAACAAGCGTTAGAAACTGTGCAGAGGCTGCTGCCTGTGCT |
| | CTGCCAAGCCCATGGTCTTACCCCGGAACAGGTTGTGGCGATTGCAT |
| | CCAACAACGGTGGAAAACAGGCCCTTGAAACGGTGCAGCGGCTCCTT |
| | CCTGTCCTGTGCCAAGCCCATGGACTCACCCCTGAACAGGTCGTGGCT |
| | ATTGCCTCCAACGGAGGCGGCAAGCAGGCCCTGGAGACAGTCCAGA |
| | GACTGCTGCCCGTGCTGTGTCAAGCTCACGGTCTGACTCCGGAACAG |
| | GTGGTTGCTATCGCTTCGCACGACGGCGGAAAACAAGCTCTTGAAAC |
| | CGTGCAAAGGCTGCTCCCGGTGCTTTGCCAAGCTCATGGCTTGACCCC |
| | AGAGCAAGTCGTGGCAATCGCGTCCAATATCGGTGGGAAGCAGGCTC |
| | TTGAGACAGTGCAAAGACTCCTCCCCGTCCTCTGTCAAGCGCACGGG |
| | CTCACTCCTGAACAGGTCGTCGCTATTGCTTCGAACAACGGGGGCAA |
| | ACAGGCACTAGAGACTGTCCAAAGACTTTTACCTGTCCTCTGCCAGG |
| | CACACGGATTGACTCCCGAACAAGTCGTCGCGATTGCCTCAAGACAC |
| | GGGGGCAAGCAAGCGCTGGAGACTGTTCAGCGACTGCTTCCTGTGCT |
| | TTGCCAGGCCCACGGTTTGACCCCTGAGCAAGTTGTGGCCATCGCCTC |
| | TAACGGAGGTGGAAAGCAGGCCTTGGAAACAGTGCAGAGACTGTTG |
| | CCCGTCTTGTGCCAGGCTCATGGGCTTACGCCTGAACAAGTCGTCGCC |
| | ATCGCTTCGAACGGAGGAGGGAAACAGGCGCTGGAAACTGTGCAGC |
| | GACTCCTTCCGGTCCTTTGTCAAGCACACGGCCTTACGCCGGAACAA |
| | GTGGTTGCCATCGCAAGCCACGACGGAGGACGCCCTGCCCTCGAGTC |
| | CATTGTGGCCCAGCTGTCACGCCCAGATCCTGCACTGGCCGCTCTTAC |
| | CAACGATCACCTGGTCGCTCTCGCGTGCCTGGGAGGAAGGCCTGCCC |
| | TGGATGCCGTCAAGAAGGGACTGCCTCATGCCCCTGCTTTGATTAAG |
| | AGGACTAATCGGCGCATCCCCGAACGGACCAGCCACAGGGTGGCATC |
| | TGGGAGCGGAGGAGGATCCGGCGGAGACGCCAAGAGCCTGACCGCC |
| | TGGAGCCGGACCCTGGTGACCTTCAAGGACGTGTTCGTGGACTTCAC |
| | CCGGGAGGAGTGGAAGCTGCTGGACACCGCCCAGCAGATCCTGTACC |
| | GGAACGTGATGCTGGAGAACTACAAGAACCTGGTGAGCCTGGGCTAC |
| | CAGCTGACCAAGCCCGACGTGATCCTGCGGCTGGAGAAGGGCGAGG |
| | AGCCCTGGCTGGTGGAGCGGGAGATCCACCAGGAAACCCACCCCGAC |
| | AGCGAAACCGCCTTCGAGATCAAGAGCAGCGTGAGCGGCGGCAAGC |
| | GCCCAGCGGCTACAAAGAAGGCCGGACAGGCCAAGAAGAAGAAGGG |
| | TTCGTACCCCTACGACGTGCCTGACTATGCCTGA |
| OEC053 | ATGGCCCCAAAGAAGAAGCGGAAAGTCGGAGGTTCAGGCGGCTCGC |
| TAL34-KRAB | ACATGGTGGACCTGAGAACTCTGGGTTACTCCCAGCAACAGCAAGAA |
| (SEQ ID NO: 445) | AAAATCAAGCCAAAAGTGCGCAGCACTGTCGCGCAGCACCACGAGG |
| | CCCTTGTGGGGCACGGGTTCACCCACGCTCACATCGTGGCCCTGAGC |
| | CAGCATCCGGCAGCCCTGGGAACCGTCGCAGTGAAGTACCAAGACAT |
| | GATCGCCGCCTTGCCGGAAGCCACTCACGAAGCAATTGTGGGAGTGG |
| | GGAAGAGAGGAGCCGGCGCAAGAGCCCTCGAAGCGCTTCTGACTGT |
| | GGCCGGAGAATTGAGAGGCCCCCCACTGCAGCTGGATACCGGACAAC |
| | TGCTGAAGATCGCCAAACGGGGTGGAGTGACTGCCGTCGAGGCTGTG |
| | CACGCCTGGAGAAACGCCTTGACCGGAGCGCCACTGAACCTTACCCC |
| | GGAGCAAGTGGTGGCGATAGCATCAAATATTGGCGGAAAGCAGGCC |
| | CTGGAAACCGTACAAAGACTGTTGCCGGTCCTGTGTCAAGCTCATGG |
| | TTTGACCCCAGAACAGGTCGTGGCTATTGCATCGAACGGCGGAGGCA |
| | AACAAGCCCTCGAGACAGTCCAGAGACTTCTGCCGGTTCTGTGCCAG |
| | GCGCACGGACTTACCCCTGAACAGGTGGTGGCGATTGCGAGCCACGA |
| | CGGTGGTAAACAGGCGTTGGAAACTGTGCAGCGGTTGCTTCCGGTGC |
| | TGTGTCAGGCTCACGGGCTGACCCCCGAACAAGTGGTAGCTATCGCC |
| | TCCCACGACGGAGGGAAGCAGGCTCTGGAAACGGTGCAACGCCTCCT |
| | TCCCGTCTTGTGCCAAGCACATGGACTGACTCCAGAACAGGTCGTCG |
| | CCATTGCCTCCCATGATGGGGGGAAGCAGGCACTGGAAACTGTACAA |
| | CGCCTGCTGCCTGTCCTCTGCCAAGCGCACGGTTTGACGCCGGAACA |
| | GGTTGTCGCCATCGCCTCGAACGGAGGGGGAAAACAGGCCCTTGAAA |
| | CAGTGCAACGGCTGCTGCCAGTGCTGTGTCAGGCGCATGGCTTGACC |
| | CCGGAGCAAGTGGTGGCCATAGCCTCGAATGGAGGAGGAAAGCAGG |
| | CCCTGGAAACCGTTCAGCGCCTCCTCCCTGTGCTGTGTCAGGCCCATG |
| | GGCTCACTCCCGAACAGGTAGTCGCCATAGCGTCGAACATCGGAGGA |
| | AAACAAGCGCTCGAAACTGTCCAAAGACTGCTTCCAGTCCTTTGCCA |
| | GGCTCATGGACTAACTCCAGAGCAAGTAGTGGCCATCGCGAGCCATG |
| | ACGGCGGCAAGCAAGCACTCGAAACCGTGCAGAGGCTGCTTCCCGTC |
| | CTGTGCCAAGCTCACGGCCTGACGCCTGAACAGGTGGTGGCTATCGC |
| | CAGCAACGGTGGTGGAAAGCAGGCGCTCGAGACTGTGCAGAGGTTG |
| | CTCCCAGTGCTCTGTCAGGCACATGGCCTCACCCCGGAGCAAGTGGT |
| | GGCAATCGCCTCAAACATCGGTGGCAAACAGGCACTTGAAACCGTGC |
| | AGCGCCTTTTGCCTGTGCTCTGCCAGGCCCACGGACTGACCCCTGAGC |
| | AAGTCGTGGCTATTGCCTCTAACGGCGGTGGCAAGCAGGCCTTGGAA |
| | ACCGTCCAGAGGCTCTTGCCGGTGCTCTGCCAGGCACACGGACTCAC |
| | TCCGGAACAGGTGGTCGCCATCGCATCGAATATCGGCGGAAAACAGG |
| | CCCTCGAAACGGTCCAGCGGTTACTCCCCGTGCTTTGTCAGGCTCACG |
| | GATTGACTCCCGAGCAAGTCGTGGCCATTGCTTCCAACATTGGGGGG |
| | AAACAGGCGCTGGAAACGGTGCAGCGACTCCTTCCTGTGTTGTGTCA |
| | AGCCCACGGCCTCACTCCAGAGCAAGTCGTGGCAATTGCCTCCAACA |
| | TCGGGGGAAAGCAAGCCTTGGAGACAGTACAGCGGCTGCTTCCTGTG |
| | CTTTGCCAAGCCCATGGACTCACCCCCGAACAAGTGGTTGCCATTGCC |
| | AGCCACGATGGAGGGAAACAAGCCCTGGAGACAGTGCAAAGGCTCC |
| | TGCCCGTGCTGTGCCAGGCACACGGCTTGACTCCTGAACAAGTCGTC |
| | GCTATCGCTTCCAATGGTGGTGGGAAGCAAGCTCTGGAGACTGTCCA |
| | GCGGCTCCTGCCAGTGTTGTGTCAAGCACACGGTCTGACCCCGGAAC |
| | AAGTCGTTGCAATCGCGTCGCATGATGGAGGACGCCCTGCCCTCGAG |
| | TCCATTGTGGCCCAGCTGTCACGCCCAGATCCTGCACTGGCCGCTCTT |
| | ACCAACGATCACCTGGTCGCTCTCGCGTGCCTGGGAGGAAGGCCTGC |
| | CCTGGATGCCGTCAAGAAGGGACTGCCTCATGCCCCTGCTTTGATTAA |
| | GAGGACTAATCGGCGCATCCCCGAACGGACCAGCCACAGGGTGGCAT |
| | CTGGGAGCGGAGGAGGATCCGGCGGAGACGCCAAGAGCCTGACCGC |
| | CTGGAGCCGGACCCTGGTGACCTTCAAGGACGTGTTCGTGGACTTCA |
| | CCCGGGAGGAGTGGAAGCTGCTGGACACCGCCCAGCAGATCCTGTAC |
| | CGGAACGTGATGCTGGAGAACTACAAGAACCTGGTGAGCCTGGGCTA |
| | CCAGCTGACCAAGCCCGACGTGATCCTGCGGCTGGAGAAGGGCGAG |
| | GAGCCCTGGCTGGTGGAGCGGGAGATCCACCAGGAAACCCACCCCG |
| | ACAGCGAAACCGCCTTCGAGATCAAGAGCAGCGTGAGCGGCGGCAA |
| | GCGCCCAGCGGCTACAAAGAAGGCCGGACAGGCCAAGAAGAAGAAG |
| | GGTTCGTACCCCTACGACGTGCCTGACTATGCCTGA |
| OEC043 | ATGGCCCCAAAGAAGAAGAGAAAGGTCGGAATTCATGGCGTGCCCG |
| ZF05-KRAB- | CAGCCGGCAGCAGCGGTTCCCTGGAGCCCGGAGAGAAACCGTATAA |
| tPT2A-TAL14-KRAB | GTGCCCGGAATGTGGAAAGTCCTTCTCCCAATCCGGCCACCTGACTG |
| (SEQ ID NO: 446) | AGCATCAGCGCACCCACACTGGCGAAAAGCCGTACAAGTGCCCTGAG |
| | TGCGGGAAGTCGTTTAGCCAGCGGGCGAACTTGCGCGCTCATCAGCG |
| | GACTCATACCGGGGAGAAGCCCTATAAATGCCCGGAGTGCGGAAAG |
| | AGCTTCAGCACCAAGAACTCCCTGACTGAACACCAGCGGACCCACAC |
| | CGGAGAGAAGCCTTACAAGTGTCCTGAATGCGGGAAATCCTTCTCCA |
| | CCTCCGGTTCACTCGTGCGCCACCAGAGAACGCACACCGGTGAAAAG |
| | CCTTATAAGTGTCCAGAGTGCGGCAAAAGCTTCAGCCAGTCCGGCAA |
| | CCTCACCGAGCACCAACGGACTCACACTGGAGAAAAGCCCTACAAAT |
| | GTCCGGAATGCGGCAAATCATTCTCGCAAGCCGGTCACCTTGCCTCG |
| | CACCAGCGCACACACACCGGGGAAAAGCCATACAAATGCCCCGAAT |
| | GCGGAAAGTCGTTCTCTCAATCCGGCGACCTGAGAAGGCATCAGAGG |
| | ACCCATACCGGAAAGAAAACCTCCGCCAGCGGATCAGGAGGTGGAT |
| | CAGGCGGAGATGCCAAGTCACTGACCGCATGGTCCCGGACCCTTGTG |
| | ACCTTCAAGGACGTGTTCGTGGACTTCACCAGAGAGGAGTGGAAGCT |
| | GCTGGACACCGCGCAACAGATTCTCTACCGGAACGTGATGCTGGAGA |
| | ACTACAAGAATCTGGTGTCCCTGGGCTACCAGCTGACCAAGCCGGAC |
| | GTGATCCTCCGCCTCGAAAAGGGGGAGGAGCCTTGGCTCGTGGAGCG |
| | GGAAATCCACCAGGAGACTCACCCGGACTCCGAGACTGCCTTCGAAA |
| | TCAAGTCATCGGTGTCCGGTGGAAAGCGGCCTGCAGCGACTAAGAAG |
| | GCCGGACAGGCCAAGAAGAAGAAGGGCTCAGGCTCAGCAACCAACT |
| | TCTCCTTGCTCAAACAAGCCGGGGACGTCGAAGAGAACCCCGGACCG |
| | ACTAGCGCCGGAAAGCTGGGTTCCGGAGAGGGTCGAGGTTCCCTCCT |
| | TACCTGTGGAGATGTGGAAGAAAACCCAGGACCCCTGGAGGGCTCCA |
| | GCGGTAGCGGAAGCCCGAAGAAGAAGCGGAAGGTCGGAATCCACGG |
| | AGTGCCTGCCGCTGGAAGCTCGGGTTCCCACATGGTCGATCTTCGCAC |
| | CCTCGGGTACTCGCAGCAGCAACAGGAAAAGATCAAGCCGAAAGTC |
| | AGGTCCACCGTCGCCCAGCACCACGAGGCACTGGTCGGCCACGGATT |
| | CACCCACGCCCACATCGTCGCGCTGTCTCAACATCCAGCCGCCCTGG |
| | GAACTGTCGCCGTCAAGTACCAAGATATGATCGCGGCACTGCCTGAA |
| | GCAACCCATGAGGCCATCGTGGGTGTGGGAAAGAGAGGAGCAGGGG |
| | CTAGAGCCCTGGAAGCGCTCCTGACCGTGGCGGGAGAGCTTAGAGGC |
| | CCTCCCCTGCAATTGGACACCGGCCAACTTCTGAAGATTGCAAAAAG |
| | GGGTGGAGTGACTGCCGTGGAAGCAGTGCACGCTTGGCGGAACGCAC |
| | TGACTGGAGCACCCCTGAATTTGACCCCCGAGCAAGTGGTTGCCATC |
| | GCAAGCAACATCGGAGGGAAACAAGCACTGGAAACTGTGCAGCGCC |
| | TGCTTCCGGTCCTGTGCCAAGCTCACGGACTGACTCCTGAACAGGTTG |
| | TGGCCATCGCGTCGCATGACGGGGGAAAACAGGCGCTCGAAACCGTC |
| | CAAAGACTGCTCCCGGTGCTGTGTCAGGCTCACGGTTTGACCCCAGA |
| | ACAAGTIGTCGCTATCGCCTCACACGACGGCGGGAAGCAAGCTCTGG |
| | AGACTGTGCAAAGACTCCTTCCTGTGCTGTGCCAAGCTCATGGATTGA |
| | CCCCGGAACAGGTCGTCGCCATCGCTTCCAATGGCGGAGGTAAACAG |
| | GCCTTGGAAACGGTGCAGAGGCTGCTGCCAGTCTTGTGTCAGGCGCA |
| | CGGGCTGACTCCAGAGCAAGTAGTAGCCATCGCCAGCAATGGAGGCG |
| | GTAAACAAGCGCTCGAGACCGTCCAGCGCTTGTTGCCAGTGTTGTGC |
| | CAGGCCCATGGGCTGACGCCCGAACAGGTGGTGGCCATTGCATCCAA |
| | CATTGGGGGGAAACAGGCGTTGGAGACAGTCCAACGGCTTCTGCCCG |
| | TCCTTTGTCAGGCACACGGACTCACTCCTGAACAAGTCGTTGCCATTG |
| | CCTCGAATATCGGGGGTAAGCAAGCCCTCGAGACTGTGCAGAGATTG |
| | TTGCCTGTCCTGTGTCAAGCGCATGGTCTGACTCCCGAACAGGTGGTG |
| | GCGATTGCTTCGAACAATGGAGGAAAACAAGCATTGGAAACCGTCCA |
| | GAGGCTGTTACCAGTCCTTTGCCAGGCACATGGACTGACCCCGGAAC |
| | AGGTCGTCGCAATCGCATCGAACATTGGAGGCAAGCAGGCTCTCGAA |
| | ACGGTCCAGAGACTGCTGCCTGTGCTCTGCCAAGCCCACGGCCTCAC |
| | TCCGGAGCAAGTCGTGGCAATCGCCTCAAACATCGGTGGAAAGCAGG |
| | CCCTTGAGACTGTCCAGCGACTGCTCCCAGTGCTCTGCCAGGCGCATG |
| | GACTCACACCCGAGCAGGTGGTCGCCATTGCTAGCAACAACGGAGGC |
| | AAACAAGCCCTGGAGACAGTGCAACGCTTGCTGCCCGTGCTTTGTCA |
| | AGCGCACGGACTTACTCCGGAACAAGTAGTGGCTATTGCGAGTAGGC |
| | ACGGCGGTAAGCAAGCCCTGGAGACAGTGCAGCGGCTTTTGCCGGTG |
| | CTCTGTCAGGCCCACGGGCTCACACCGGAACAGGTCGTAGCTATTGC |
| | GTCCCATGACGGCGGAAAGCAAGCCTTAGAAACTGTGCAGCGGTTGC |
| | TGCCTGTTCTGTGCCAGGCTCATGGGCTCACTCCCGAGCAAGTGGTCG |
| | CGATTGCGAGCAACGGAGGTGGCAAGCAAGCACTAGAAACCGTGCA |
| | ACGACTGCTGCCGGTCTTGTGCCAAGCGCATGGCCTGACACCCGAAC |
| | AGGTGGTGGCTATCGCGTCCAACAACGGGGGCAAACAGGCCCTGGA |
| | AACAGTCCAAAGGCTGTTGCCGGTCCTCTGTCAAGCTCACGGACTAA |
| | CCCCCGAACAGGTGGTGGCAATAGCTTCGCGGCATGGGGGCAAGCAG |
| | GCACTCGAAACAGTGCAGAGGTTGCTCCCGGTACTTTGCCAAGCACA |
| | TGGCTTGACCCCTGAGCAAGTCGTGGCCATCGCATCCAACGGCGGAG |
| | GGAAGCAAGCGCTTGAAACTGTCCAACGCCTCCTGCCCGTGTTGTGT |
| | CAAGCCCATGGCCTTACGCCTGAACAAGTCGTCGCTATTGCAAGTAA |
| | CGGAGGAGGGAGGCCTGCCCTGGAGTCCATTGTCGCCCAACTGTCGC |
| | GACCCGATCCAGCCCTGGCAGCCCTCACTAACGACCATCTCGTGGCG |
| | CTGGCATGCCTCGGTGGTAGACCAGCTCTGGACGCCGTGAAGAAGGG |
| | ACTTCCTCATGCCCCCGCACTGATCAAGCGCACTAACCGGAGGATCC |
| | CTGAAAGAACCTCCCACAGAGTGGCATCCGGTTCAGGAGGAGGGTCC |
| | GGAGGAGCTAGGGATGATGCCAAGAGCTTGACCGCCTGGTCACGGAC |
| | TCTTGTCACTTTCAAGGATGTCTTTGTGGACTTTACCAGAGAAGAATG |
| | GAAGCTCCTGGACACTGCGCAGCAGATCCTGTACAGAAACGTGATGT |
| | TGGAAAACTACAAAAACCTGGTGTCACTCGGGTACCAACTCACCAAG |
| | CCCGACGTCATCCTGCGCCTGGAAAAGGGCGAAGAACCGTGGTTGGT |
| | CGAGAGAGAAATCCATCAGGAAACCCACCCTGACTCGGAAACCGCCT |
| | TCGAGATCAAGTCCTCGGTGCCTAGCTCCGGAGGAAAAAGGCCCGCC |
| | GCAACCAAGAAGGCTGGGCAGGCTAAGAAAAAGAAGGGGTCCGGCT |
| | CCTAATGA |

TABLE 13
Sequences: Epigenetic Modifying Agent mRNA

| Name | Nucleic Acid Sequence |
|---|---|
| MR250 | AGGAAAUAAGAGAGAAAAGAAGAGUAAGAAGAAAUAUAAGAGCCA |
| ZF05-KRAB | CCAUGGCCCCCAAGAAGAAGCGGAAGGUGGGCAUCCACGGCGUGC |
| (SEQ ID NO: | CCGCCGCCGGCAGCAGCGGAUCCCUGGAGCCCGGAGAGAAACCGU |
| 447) | AUAAGUGCCCGGAAUGUGGAAAGUCCUUCUCCCAAUCCGGCCACC |
| | UGACUGAGCAUCAGCGCACCCACACUGGCGAAAAGCCGUACAAGU |
| | GCCCUGAGUGCGGGAAGUCGUUUAGCCAGCGGGCGAACUUGCGCG |
| | CUCAUCAGCGGACUCAUACCGGGGAGAAGCCCUAUAAAUGCCCGG |
| | AGUGCGGAAAGAGCUUCAGCACCAAGAACUCCCUGACUGAACACC |
| | AGCGGACCCACACCGGAGAGAAGCCUUACAAGUGUCCUGAAUGCG |
| | GGAAAUCCUUCUCCACCUCCGGUUCACUCGUGCGCCACCAGAGAA |
| | CGCACACCGGUGAAAAGCCUUAUAAGUGUCCAGAGUGCGGCAAAA |
| | GCUUCAGCCAGUCCGGCAACCUCACCGAGCACCAACGGACUCACA |
| | CUGGAGAAAAGCCCUACAAAUGUCCGGAAUGCGGCAAAUCAUUCU |
| | CGCAAGCCGGUCACCUUGCCUCGCACCAGCGCACACACACCGGGG |
| | AAAAGCCAUACAAAUGCCCCGAAUGCGGAAAGUCGUUCUCUCAAU |
| | CCGGCGACCUGAGAAGGCAUCAGAGGACCCAUACCGGAAAGAAAA |
| | CCUCCGCUUCCGGAUCCGGUGGAGGAUCAGGCGGCGAUGCGAAGU |
| | CCCUCACCGCUUGGUCACGGACCCUAGUGACCUUCAAAGACGUGU |
| | UCGUGGAUUUCACCAGGGAAGAAUGGAAGCUCCUGGACACCGCCC |
| | AGCAAAUACUGUACCGGAACGUCAUGCUGGAGAACUAUAAGAACC |
| | UGGUCAGCUUGGGCUACCAGCUCACCAAGCCGGACGUGAUCCUGC |
| | GGCUGGAGAAGGGAGAGGAGCCUUGGCUCGUGGAAAGGGAAAUUC |
| | AUCAGGAGACUCACCCUGACAGCGAAACCGCCUUCGAGAUUAAGU |
| | CCUCCGUGUCCGGAGGAAAGCGGCCCGCCGCCACCAAGAAGGCCG |
| | GCCAGGCCAAGAAGAAGAAGGGCAGCUACCCCUACGACGUGCCCG |
| | ACUACGCCUGAGCGGCCGCUUAAUUAAGCUGCCUUCUGCGGGGCU |
| | UGCCUUCUGGCCAUGCCCUUCUUCUCUCCCUUGCACCUGUACCUC |
| | UUGGUCUUUGAAUAAAGCCUGAGUAGGAAGUCUAGAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAA |
| MR319 | AGGAAAUAAGAGAGAAAAGAAGAGUAAGAAGAAAUAUAAGAGCCA |
| ZF07-KRAB | CCAUGGCCCCCAAGAAGAAGCGGAAGGUGGGCAUCCACGGCGUGC |
| (SEQ ID NO: | CCGCCGCCGGCAGCAGCGGAUCCCUCGAGCCUGGCGAAAAGCCUU |
| 448) | AUAAGUGCCCAGAAUGCGGAAAGAGCUUCUCCGACAAGAAGGAUC |
| | UGACCCGCCACCAAAGGACCCACACUGGGGAGAAGCCUUACAAGU |
| | GUCCGGAGUGCGGAAAGUCCUUUUCGCAAUCGUCCAGCCUGGUCC |
| | GGCACCAGAGGACGCACACCGGAGAGAAGCCAUACAAAUGCCCGG |
| | AGUGUGGGAAGUCGUUCUCCACCACUGGAAACCUCACUGUGCACC |
| | AGCGCACGCAUACUGGGGAAAAGCCCUAUAAGUGUCCAGAGUGCG |
| | GGAAGUCCUUCUCAACCUCCGGCGAAUUGGUCAGACACCAGCGGA |
| | CACACACCGGCGAGAAGCCCUACAAGUGCCCUGAGUGCGGCAAAA |
| | GCUUCAGCACUACCGGCAACCUGACUGUGCAUCAGCGGACCCAUA |
| | CCGGAGAAAAACCCUACAAAUGUCCCGAAUGCGGCAAAUCCUUCU |
| | CCACCUCCCACUCGCUGACCGAACACCAGAGAACCCAUACUGGAG |
| | AAAAGCCGUACAAGUGCCCGGAAUGUGGAAAGUCUUUCAGCACCU |
| | CCGGAAACCUUGUGCGGCAUCAGCGCACUCACACCGGCAAAAAGA |
| | CCUCAGCUUCCGGAUCCGGUGGAGGAUCAGGCGGCGAUGCGAAGU |
| | CCCUCACCGCUUGGUCACGGACCCUAGUGACCUUCAAAGACGUGU |
| | UCGUGGAUUUCACCAGGGAAGAAUGGAAGCUCCUGGACACCGCCC |
| | AGCAAAUACUGUACCGGAACGUCAUGCUGGAGAACUAUAAGAACC |
| | UGGUCAGCUUGGGCUACCAGCUCACCAAGCCGGACGUGAUCCUGC |
| | GGCUGGAGAAGGGAGAGGAGCCUUGGCUCGUGGAAAGGGAAAUUC |
| | AUCAGGAGACUCACCCUGACAGCGAAACCGCCUUCGAGAUUAAGU |
| | CCUCCGUGUCCGGAGGAAAGCGGCCCGCCGCCACCAAGAAGGCCG |
| | GCCAGGCCAAGAAGAAGAAGGGCAGCUACCCCUACGACGUGCCCG |
| | ACUACGCCUGAGCGGCCGCUUAAUUAAGCUGCCUUCUGCGGGGCU |
| | UGCCUUCUGGCCAUGCCCUUCUUCUCUCCCUUGCACCUGUACCUC |
| | UUGGUCUUUGAAUAAAGCCUGAGUAGGAAGUCUAGAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAA |
| MR340 | AGGAAAUAAGAGAGAAAAGAAGAGUAAGAAGAAAUAUAAGAGCCA |
| ZF05-MQ1 | CCAUGGCCCCCAAGAAGAAGCGGAAGGUGGGCAUCCACGGCGUGC |
| (SEQ ID NO: | CCGCCGCCGGCAGCAGCGGAUCCCUGGAGCCCGGAGAGAAACCGU |
| 449) | AUAAGUGCCCGGAAUGUGGAAAGUCCUUCUCCCAAUCCGGCCACC |
| | UGACUGAGCAUCAGCGCACCCACACUGGCGAAAAGCCGUACAAGU |
| | GCCCUGAGUGCGGGAAGUCGUUUAGCCAGCGGGCGAACUUGCGCG |
| | CUCAUCAGCGGACUCAUACCGGGGAGAAGCCCUAUAAAUGCCCGG |
| | AGUGCGGAAAGAGCUUCAGCACCAAGAACUCCCUGACUGAACACC |
| | AGCGGACCCACACCGGAGAGAAGCCUUACAAGUGUCCUGAAUGCG |
| | GGAAAUCCUUCUCCACCUCCGGUUCACUCGUGCGCCACCAGAGAA |
| | CGCACACCGGUGAAAAGCCUUAUAAGUGUCCAGAGUGCGGCAAAA |
| | GCUUCAGCCAGUCCGGCAACCUCACCGAGCACCAACGGACUCACA |
| | CUGGAGAAAAGCCCUACAAAUGUCCGGAAUGCGGCAAAUCAUUCU |
| | CGCAAGCCGGUCACCUUGCCUCGCACCAGCGCACACACACCGGGG |
| | AAAAGCCAUACAAAUGCCCCGAAUGCGGAAAGUCGUUCUCUCAAU |
| | CCGGCGACCUGAGAAGGCAUCAGAGGACCCAUACCGGAAAGAAAA |
| | CCUCCGCAUCUGGGAGCGGAGGAGGAUCCGGCGGAGCCAGAGACU |
| | CCAAGGUCGAGAACAAGACCAAGAAGCUGCGGGUGUUCGAGGCCU |
| | UUGCCGGGAUCGGAGCACAGCGGAAGGCCUUGGAGAAGGUCCGCA |
| | AGGACGAAUACGAAAUCGUGGGCCUUGCCGAGUGGUACGUGCCAG |
| | CCAUCGUGAUGUACCAGGCUAUCCACAACAACUUCCACACCAAGC |
| | UGGAGUACAAGUCCGUGAGCAGAGAAGAGAUGAUCGACUACCUCG |
| | AAAACAAGACCCUGUCCUGGAACUCCAAGAAUCCCGUGUCGAACG |
| | GUUACUGGAAGCGCAAGAAGGAUGAUGAACUGAAGAUUAUCUACA |
| | ACGCGAUUAAGCUGUCCGAAAAGGAAGGAAACAUUUUCGACAUUC |
| | GCGAUCUGUACAAGAGAACCCUCAAGAACAUUGAUCUGCUUACCU |
| | ACUCCUUCCCGUGCCAAGAUCUCAGCCAGCAGGGGAUUCAGAAGG |
| | GGAUGAAGCGGGGUUCAGGGACCAGAUCAGGCCUGCUCUGGGAGA |
| | UCGAAAGAGCCCUGGACUCCACCGAGAAGAACGAUCUCCCGAAAU |
| | ACCUUCUGAUGGAGAACGUCGGCGCUCUCCUGCACAAGAAGAACG |
| | AAGAAGAACUGAACCAGUGGAAGCAGAAGCUGGAGUCACUCGGAU |
| | ACCAGAAUUCUAUUGAGGUGCUGAACGCGGCCGAUUUCGGAUCCU |
| | CGCAAGCCAGGCGGAGAGUGUUCAUGAUUUCGACCCUGAACGAGU |
| | UCGUGGAGCUGCCUAAGGGCGACAAGAAGCCCAAGUCCAUCAAGA |
| | AGGUCCUGAACAAGAUUGUCAGCGAAAAGGACAUCCUCAACAAUC |
| | UGCUUAAGUACAACCUGACCGAGUUUAAAAAGACCAAAAGCAACA |
| | UCAACAAGGCCUCCCUGAUCGGAUACUCCAAGUUCAAUUCCGAAG |
| | GCUACGUGUACGACCCGGAAUUCACCGGACCAACCUUGACCGCCU |
| | CCGGUGCUAACUCCCGGAUCAAGAUCAAGGACGGAUCCAACAUCC |
| | GGAAGAUGAACUCCGACGAGACUUUUCUGUACAUCGGUUUCGACU |
| | CCCAAGACGGAAAGCGCGUGAACGAGAUAGAGUUCCUCACCGAGA |
| | AUCAGAAGAUCUUUGUGUGCGGCAACUCCAUUAGCGUGGAGGUGC |
| | UGGAGGCCAUCAUCGAUAAGAUCGGUGGCCCUUCCUCCGGUGGAA |
| | AGCGCCCAGCGGCUACAAAGAAGGCCGGACAGGCCAAGAAGAAGA |
| | AGGGUUCGUACCCCUACGACGUGCCUGACUAUGCCUGAGCGGCCG |
| | CUUAAUUAAGCUGCCUUCUGCGGGGCUUGCCUUCUGGCCAUGCCC |
| | UUCUUCUCUCCCUUGCACCUGUACCUCUUGGUCUUUGAAUAAAGC |
| | CUGAGUAGGAAGUCUAGAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAA |
| MR353 | AGGAAAUAAGAGAGAAAAGAAGAGUAAGAAGAAAUAUAAGAGCCA |
| ZF07-MQ1 | CCAUGGCCCCCAAGAAGAAGCGGAAGGUGGGCAUCCACGGCGUGC |
| (SEQ ID NO: | CCGCCGCCGGCAGCAGCGGAUCCCUCGAGCCUGGCGAAAAGCCUU |
| 450) | AUAAGUGCCCAGAAUGCGGAAAGAGCUUCUCCGACAAGAAGGAUC |
| | UGACCCGCCACCAAAGGACCCACACUGGGGAGAAGCCUUACAAGU |
| | GUCCGGAGUGCGGAAAGUCCUUUUCGCAAUCGUCCAGCCUGGUCC |
| | GGCACCAGAGGACGCACACCGGAGAGAAGCCAUACAAAUGCCCGG |
| | AGUGUGGGAAGUCGUUCUCCACCACUGGAAACCUCACUGUGCACC |
| | AGCGCACGCAUACUGGGGAAAAGCCCUAUAAGUGUCCAGAGUGCG |
| | GGAAGUCCUUCUCAACCUCCGGCGAAUUGGUCAGACACCAGCGGA |
| | CACACACCGGCGAGAAGCCCUACAAGUGCCCUGAGUGCGGCAAAA |
| | GCUUCAGCACUACCGGCAACCUGACUGUGCAUCAGCGGACCCAUA |
| | CCGGAGAAAAACCCUACAAAUGUCCCGAAUGCGGCAAAUCCUUCU |
| | CCACCUCCCACUCGCUGACCGAACACCAGAGAACCCAUACUGGAG |
| | AAAAGCCGUACAAGUGCCCGGAAUGUGGAAAGUCUUUCAGCACCU |
| | CCGGAAACCUUGUGCGGCAUCAGCGCACUCACACCGGCAAAAAGA |
| | CCUCAGCAUCUGGGAGCGGAGGAGGAUCCGGCGGAGCCAGAGACU |
| | CCAAGGUCGAGAACAAGACCAAGAAGCUGCGGGUGUUCGAGGCCU |
| | UUGCCGGGAUCGGAGCACAGCGGAAGGCCUUGGAGAAGGUCCGCA |
| | AGGACGAAUACGAAAUCGUGGGCCUUGCCGAGUGGUACGUGCCAG |
| | CCAUCGUGAUGUACCAGGCUAUCCACAACAACUUCCACACCAAGC |
| | UGGAGUACAAGUCCGUGAGCAGAGAAGAGAUGAUCGACUACCUCG |
| | AAAACAAGACCCUGUCCUGGAACUCCAAGAAUCCCGUGUCGAACG |
| | GUUACUGGAAGCGCAAGAAGGAUGAUGAACUGAAGAUUAUCUACA |
| | ACGCGAUUAAGCUGUCCGAAAAGGAAGGAAACAUUUUCGACAUUC |
| | GCGAUCUGUACAAGAGAACCCUCAAGAACAUUGAUCUGCUUACCU |
| | ACUCCUUCCCGUGCCAAGAUCUCAGCCAGCAGGGGAUUCAGAAGG |
| | GGAUGAAGCGGGGUUCAGGGACCAGAUCAGGCCUGCUCUGGGAGA |
| | UCGAAAGAGCCCUGGACUCCACCGAGAAGAACGAUCUCCCGAAAU |
| | ACCUUCUGAUGGAGAACGUCGGCGCUCUCCUGCACAAGAAGAACG |
| | AAGAAGAACUGAACCAGUGGAAGCAGAAGCUGGAGUCACUCGGAU |
| | ACCAGAAUUCUAUUGAGGUGCUGAACGCGGCCGAUUUCGGAUCCU |
| | CGCAAGCCAGGCGGAGAGUGUUCAUGAUUUCGACCCUGAACGAGU |
| | UCGUGGAGCUGCCUAAGGGCGACAAGAAGCCCAAGUCCAUCAAGA |
| | AGGUCCUGAACAAGAUUGUCAGCGAAAAGGACAUCCUCAACAAUC |
| | UGCUUAAGUACAACCUGACCGAGUUUAAAAAGACCAAAAGCAACA |
| | UCAACAAGGCCUCCCUGAUCGGAUACUCCAAGUUCAAUUCCGAAG |
| | GCUACGUGUACGACCCGGAAUUCACCGGACCAACCUUGACCGCCU |
| | CCGGUGCUAACUCCCGGAUCAAGAUCAAGGACGGAUCCAACAUCC |
| | GGAAGAUGAACUCCGACGAGACUUUUCUGUACAUCGGUUUCGACU |
| | CCCAAGACGGAAAGCGCGUGAACGAGAUAGAGUUCCUCACCGAGA |
| | AUCAGAAGAUCUUUGUGUGCGGCAACUCCAUUAGCGUGGAGGUGC |
| | UGGAGGCCAUCAUCGAUAAGAUCGGUGGCCCUUCCUCCGGUGGAA |
| | AGCGCCCAGCGGCUACAAAGAAGGCCGGACAGGCCAAGAAGAAGA |
| | AGGGUUCGUACCCCUACGACGUGCCUGACUAUGCCUGAGCGGCCG |
| | CUUAAUUAAGCUGCCUUCUGCGGGGCUUGCCUUCUGGCCAUGCCC |
| | UUCUUCUCUCCCUUGCACCUGUACCUCUUGGUCUUUGAAUAAAGC |
| | CUGAGUAGGAAGUCUAGAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAA |
| MR553 | AGGAAAUAAGAGAGAAAAGAAGAGUAAGAAGAAAUAUAAGAGCCA |
| ZF16-KRAB | CCAUGGCCCCCAAGAAGAAGCGGAAGGUGGGCAUCCACGGCGUGC |
| (SEQ ID NO: | CCGCCGCCGGCAGCAGCGGAUCCCUGGAGCCGGGAGAGAAGCCUU |
| 451) | AUAAGUGUCCUGAGUGCGGCAAAUCAUUCUCCCAACGCGCCCACC |
| | UCGAGAGACACCAGAGAACCCACACCGGGGAAAAGCCGUACAAGU |
| | GCCCUGAGUGUGGGAAGUCCUUUUCCCGGUCCGACCAUUUGACCA |
| | ACCAUCAGCGGACCCACACUGGGGAGAAGCCAUACAAAUGCCCAG |
| | AAUGCGGCAAAAGCUUCUCCCGGAACGACGCACUUACCGAACAUC |
| | AGCGCACGCACACCGGAGAGAAACCCUAUAAGUGCCCCGAAUGUG |
| | GAAAGUCAUUCUCCCGGGCCGACAACCUGACAGAGCACCAGCGGA |
| | CUCACACUGGAGAAAAGCCUUACAAGUGUCCCGAAUGCGGGAAGU |
| | CGUUCAGCUCGAAGAAGGCUCUGACCGAACACCAGAGGACCCAUA |
| | CCGGCGAAAAGCCCUAUAAAUGCCCGGAGUGCGGAAAGAGCUUCA |
| | GCAGGAACGAUGCCCUCACCGAGCACCAACGCACUCAUACUGGCG |
| | AAAAACCGUACAAAUGUCCGGAAUGCGGAAAGUCCUUCUCGCACA |
| | AGAACGCGCUGCAGAAUCACCAGCGCACUCACACCGGCAAAAAGA |
| | CCUCUGCUUCCGGAUCCGGUGGAGGAUCAGGCGGCGAUGCGAAGU |
| | CCCUCACCGCUUGGUCACGGACCCUAGUGACCUUCAAAGACGUGU |
| | UCGUGGAUUUCACCAGGGAAGAAUGGAAGCUCCUGGACACCGCCC |
| | AGCAAAUACUGUACCGGAACGUCAUGCUGGAGAACUAUAAGAACC |
| | UGGUCAGCUUGGGCUACCAGCUCACCAAGCCGGACGUGAUCCUGC |
| | GGCUGGAGAAGGGAGAGGAGCCUUGGCUCGUGGAAAGGGAAAUUC |
| | AUCAGGAGACUCACCCUGACAGCGAAACCGCCUUCGAGAUUAAGU |
| | CCUCCGUGUCCGGAGGAAAGCGGCCCGCCGCCACCAAGAAGGCCG |
| | GCCAGGCCAAGAAGAAGAAGGGCAGCUACCCCUACGACGUGCCCG |
| | ACUACGCCUGAGCGGCCGCUUAAUUAAGCUGCCUUCUGCGGGGCU |
| | UGCCUUCUGGCCAUGCCCUUCUUCUCUCCCUUGCACCUGUACCUC |
| | UUGGUCUUUGAAUAAAGCCUGAGUAGGAAGUCUAGAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAA |
| MR697 | AGGAAAUAAGAGAGAAAAGAAGAGUAAGAAGAAAUAUAAGAGCCA |
| ZF24-KRAB | CCAUGGCCCCCAAGAAGAAGCGGAAGGUGGGCAUCCACGGCGUGC |
| (SEQ ID NO: | CCGCCGCCGGCAGCAGCGGAUCCCUGGAACCCGGAGAGAAGCCCU |
| 452) | AUAAGUGUCCGGAAUGCGGAAAGAGUUUCAGCUCGAAGAAGCACC |
| | UCGCCGAGCACCAACGGACCCACACGGGAGAAAAGCCCUACAAGU |
| | GCCCAGAGUGCGGAAAGUCGUUCAGCCAAAACUCCACCCUGACCG |
| | AACACCAGCGGACUCAUACCGGGGAGAAACCUUACAAGUGUCCAG |
| | AAUGCGGGAAGUCGUUCUCCCAAUCCGGAGACUUGCGCAGACACC |
| | AGCGCACUCACACCGGCGAAAAACCGUACAAAUGCCCGGAGUGUG |
| | GAAAGUCAUUUUCCACCCACCUGGAUCUGAUCAGGCAUCAGCGCA |
| | CCCACACUGGCGAAAAGCCUUACAAAUGUCCGGAGUGCGGCAAAU |
| | CCUUCUCUGACCCCGGCAACCUCGUGCGCCAUCAGAGGACUCACA |
| | CUGGGGAGAAGCCUUAUAAGUGCCCCGAAUGUGGAAAGAGCUUCU |
| | CCACCUCCGGCAACCUUACCGAGCAUCAGCGGACACACACCGGGG |
| | AAAAGCCGUACAAGUGCCCUGAGUGUGGCAAAUCAUUCUCCGAAC |
| | GGAGCCAUCUGCGGGAGCACCAGAGAACCCACACCGGAAAGAAAA |
| | CUUCCGCUUCCGGAUCCGGUGGAGGAUCAGGCGGCGAUGCGAAGU |
| | CCCUCACCGCUUGGUCACGGACCCUAGUGACCUUCAAAGACGUGU |
| | UCGUGGAUUUCACCAGGGAAGAAUGGAAGCUCCUGGACACCGCCC |
| | AGCAAAUACUGUACCGGAACGUCAUGCUGGAGAACUAUAAGAACC |
| | UGGUCAGCUUGGGCUACCAGCUCACCAAGCCGGACGUGAUCCUGC |
| | GGCUGGAGAAGGGAGAGGAGCCUUGGCUCGUGGAAAGGGAAAUUC |
| | AUCAGGAGACUCACCCUGACAGCGAAACCGCCUUCGAGAUUAAGU |
| | CCUCCGUGUCCGGAGGAAAGCGGCCCGCCGCCACCAAGAAGGCCG |
| | GCCAGGCCAAGAAGAAGAAGGGCAGCUACCCCUACGACGUGCCCG |
| | ACUACGCCUGAGCGGCCGCUUAAUUAAGCUGCCUUCUGCGGGGCU |
| | UGCCUUCUGGCCAUGCCCUUCUUCUCUCCCUUGCACCUGUACCUC |
| | UUGGUCUUUGAAUAAAGCCUGAGUAGGAAGUCUAGAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAA |
| MR896 | AGGAAAUAAGAGAGAAAAGAAGAGUAAGAAGAAAUAUAAGAGCCA |
| TAL04- | CCAUGGCCCCCAAGAAGAAGCGGAAGGUGGGCAUCCACGGCGUGC |
| KRAB | CCGCCGCCGGCAGCAGCGGAUCCCACAUGGUGGACCUGAGAACCC |
| (SEQ ID NO: | UGGGAUACUCGCAGCAGCAGCAGGAAAAGAUCAAGCCCAAAGUGC |
| 453) | GGUCCACUGUGGCCCAGCACCAUGAGGCCCUCGUCGGACACGGAU |
| | UCACCCACGCGCACAUCGUGGCUCUGUCACAGCAUCCCGCCGCCU |
| | UGGGAACCGUGGCUGUGAAGUACCAGGACAUGAUUGCUGCACUUC |
| | CUGAGGCGACCCACGAAGCCAUUGUGGGAGUCGGAAAACGGGGUG |
| | CAGGCGCUAGAGCCCUGGAGGCCCUGUUAACGGUGGCAGGCGAAC |
| | UGCGGGGACCACCAUUGCAGCUCGAUACCGGCCAGCUGCUGAAGA |
| | UUGCCAAGAGGGGCGGAGUCACAGCGGUGGAGGCGGUCCAUGCCU |
| | GGCGCAACGCUCUUACUGGGGCUCCACUGAACCUGACCCCUGAAC |
| | AGGUAGUGGCCAUCGCAUCGAACAAUGGCGGUAAGCAGGCUCUCG |
| | AAACCGUCCAAAGGCUCCUUCCGGUGUUAUGCCAGGCUCAUGGGC |
| | UGACGCCUGAACAAGUGGUCGCAAUCGCAUCGCAUGACGGGGGGA |
| | AACAGGCAUUAGAAACCGUGCAACGCCUCCUUCCUGUCCUUUGUC |
| | AAGCACAUGGAUUGACUCCUGAGCAAGUUGUCGCGAUUGCCUCCA |
| | ACAUUGGCGGCAAACAGGCACUGGAAACCGUUCAACGGCUCCUCC |
| | CCGUACUGUGCCAGGCACACGGCCUGACCCCCGAACAAGUCGUCG |
| | CCAUUGCCUCGAACAUCGGAGGCAAGCAGGCGCUUGAAACCGUGC |
| | AGCGCUUGCUGCCUGUGCUCUGCCAAGCGCACGGCCUAACCCCUG |
| | AACAGGUGGUAGCAAUUGCUUCCCACGACGGUGGGAAGCAGGCCC |
| | UCGAGACAGUGCAAAGACUGUUGCCGGUCCUCUGCCAGGCCCACG |
| | GACUUACCCCCGAGCAAGUCGUGGCUAUUGCGUCCAAUAAUGGCG |
| | GAAAACAGGCACUUGAGACAGUCCAGAGGCUGCUGCCUGUCUUAU |
| | GCCAAGCCCACGGUCUUACUCCAGAACAGGUGGUCGCUAUCGCAU |
| | CAAACAUUGGAGGAAAACAGGCGCUGGAAACUGUCCAACGCCUGU |
| | UGCCCGUGCUCUGUCAGGCUCACGGGCUGACUCCCGAGCAAGUGG |
| | UGGCAAUCGCCUCCAACGGGGGAGGAAAGCAGGCCUUGGAAACCG |
| | UACAGCGGCUGUUGCCUGUGUUGUGCCAAGCUCACGGUCUGACGC |
| | CAGAGCAAGUGGUGGCCAUUGCUUCGCACGACGGCGGGAAGCAGG |
| | CACUCGAAACGGUGCAGAGGUUGCUUCCCGUCUUGUGCCAGGCGC |
| | ACGGACUCACCCCAGAACAAGUGGUCGCAAUUGCUAGCAACAUUG |
| | GUGGCAAACAAGCGCUCGAAACUGUACAACGGCUGCUCCCGGUGC |
| | UUUGCCAAGCCCACGGGCUUACCCCAGAGCAAGUGGUGGCCAUUG |
| | CGAGCAACAUCGGGGGCAAGCAAGCGCUUGAGACUGUACAAAGAC |
| | UGCUGCCCGUGCUGUGCCAAGCCCACGGCUUAACCCCGGAACAGG |
| | UCGUGGCCAUCGCCUCGAAUGGAGGUGGAAAGCAAGCCCUGGAGA |
| | CUGUGCAGCGGUUGCUGCCCGUUCUGUGUCAAGCUCACGGACUGA |
| | CACCGGAACAGGUGGUGGCUAUCGCAAGCAACAACGGCGGAAAGC |
| | AAGCCCUGGAGACUGUGCAGCGGCUGCUGCCAGUGCUGUGUCAGG |
| | CGCAUGGACUGACCCCCGAACAAGUCGUAGCCAUAGCCAGCCGCC |
| | ACGGAGGGAAGCAGGCCCUGGAAACAGUCCAGCGCCUCUUGCCCG |
| | UGUUGUGUCAAGCGCAUGGCCUCACCCCUGAACAGGUCGUUGCCA |
| | UUGCGUCCAGACAUGGAGGGAAGCAAGCACUAGAAACAGUGCAGA |
| | GACUGCUACCCGUCCUGUGCCAGGCCCAUGGACUCACGCCGGAGC |
| | AAGUGGUGGCGAUAGCAUCGAACAUUGGUGGGAAACAAGCCCUUG |
| | AAACUGUCCAGAGACUGUUACCUGUGCUUUGUCAGGCACAUGGGC |
| | UCACACCAGAACAGGUGGUCGCCAUCGCUAGCAACGGAGGGGGAA |
| | AACAGGCUUUGGAGACUGUCCAGCGGCUCCUACCGGUGCUCUGCC |
| | AAGCACACGGAUUGACGCCCGAACAGGUUGUGGCGAUUGCGUCCA |
| | ACGGUGGAGGACGGCCUGCCCUGGAGAGCAUUGUGGCUCAGCUCU |
| | CCAGACCUGACCCAGCCCUCGCAGCUCUGACUAACGAUCAUCUGG |
| | UGGCGCUGGCCUGUUUGGGGGGACGCCCUGCUUUGGACGCCGUGA |
| | AGAAGGGUCUGCCACAUGCCCCCGCACUGAUCAAGCGCACCAACC |
| | GGCGAAUCCCUGAGCGCACUUCCCAUCGAGUGGCUUCCGGAUCCG |
| | GUGGAGGAUCAGGCGGCGAUGCGAAGUCCCUCACCGCUUGGUCAC |
| | GGACCCUAGUGACCUUCAAAGACGUGUUCGUGGAUUUCACCAGGG |
| | AAGAAUGGAAGCUCCUGGACACCGCCCAGCAAAUACUGUACCGGA |
| | ACGUCAUGCUGGAGAACUAUAAGAACCUGGUCAGCUUGGGCUACC |
| | AGCUCACCAAGCCGGACGUGAUCCUGCGGCUGGAGAAGGGAGAGG |
| | AGCCUUGGCUCGUGGAAAGGGAAAUUCAUCAGGAGACUCACCCUG |
| | ACAGCGAAACCGCCUUCGAGAUUAAGUCCUCCGUGUCCGGAGGAA |
| | AGCGGCCCGCCGCCACCAAGAAGGCCGGCCAGGCCAAGAAGAAGA |
| | AGGGCAGCUACCCCUACGACGUGCCCGACUACGCCUGAGCGGCCG |
| | CUUAAUUAAGCUGCCUUCUGCGGGGCUUGCCUUCUGGCCAUGCCC |
| | UUCUUCUCUCCCUUGCACCUGUACCUCUUGGUCUUUGAAUAAAGC |
| | CUGAGUAGGAAGUCUAGAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAA |
| MR861 | AGGAAAUAAGAGAGAAAAGAAGAGUAAGAAGAAAUAUAAGAGCCA |
| TAL08- | CCAUGGCCCCCAAGAAGAAGCGGAAGGUGGGCAUCCACGGCGUGC |
| KRAB | CCGCCGCCGGCAGCAGCGGAUCCCACAUGGUGGACCUCCGGACUC |
| (SEQ ID NO: | UUGGUUACUCCCAACAGCAGCAGGAAAAGAUCAAGCCUAAGGUCC |
| 454) | GCUCCACUGUGGCCCAACACCACGAGGCCCUUGUGGGACAUGGAU |
| | UCACCCACGCCCACAUCGUGGCGCUGUCGCAGCACCCUGCUGCCU |
| | UGGGGACCGUCGCCGUGAAGUACCAGGACAUGAUCGCCGCCUUAC |
| | CCGAAGCCACCCACGAGGCAAUCGUGGGAGUGGGUAAAAGGGGUG |
| | CCGGUGCAAGAGCUCUGGAGGCCCUUCUCACCGUGGCCGGAGAAC |
| | UUAGAGGGCCUCCACUCCAGCUGGAUACCGGUCAGCUGUUGAAGA |
| | UUGCUAAGAGAGGAGGGGUGACCGCCGUGGAAGCCGUGCAUGCGU |
| | GGCGCAACGCUCUGACUGGAGCCCCACUGAACUUGACCCCCGAAC |
| | AGGUGGUCGCCAUCGCCUCCAAUAACGGGGGCAAACAGGCGCUGG |
| | AAACCGUGCAGAGAUUGCUGCCUGUCUUAUGCCAAGCACACGGAC |
| | UCACCCCCGAGCAAGUCGUGGCUAUCGCCUCCAGACACGGCGGGA |
| | AGCAAGCUCUGGAGACUGUGCAGAGACUGCUACCCGUGUUGUGUC |
| | AAGCCCACGGUCUUACUCCGGAGCAAGUGGUCGCUAUUGCCUCGC |
| | AUGAUGGUGGUAAACAGGCCCUGGAGACAGUGCAGCGCUUGCUCC |
| | CUGUGCUGUGUCAGGCACACGGCCUGACACCGGAACAGGUGGUGG |
| | CCAUCGCUUCGAACAUUGGAGGUAAACAGGCAUUGGAGACAGUCC |
| | AGAGGCUCCUGCCGGUCCUCUGCCAAGCUCACGGUCUGACUCCUG |
| | AACAAGUAGUCGCCAUUGCAUCCCACGAUGGCGGUAAGCAGGCUC |
| | UUGAAACUGUCCAGAGACUGUUACCUGUGCUCUGUCAGGCCCAUG |
| | GACUGACGCCUGAACAGGUCGUCGCGAUUGCGUCAAACAUCGGUG |
| | GAAAGCAAGCGCUUGAAACGGUCCAGCGCCUACUGCCUGUGUUGU |
| | GCCAAGCGCACGGCCUCACACCAGAACAAGUCGUCGCAAUCGCAA |
| | GCCAUGAUGGCGGAAAGCAGGCACUAGAAACUGUUCAGCGCCUUC |
| | UACCCGUCUUGUGCCAGGCUCAUGGACUCACUCCCGAACAAGUGG |
| | UGGCUAUUGCGUCCAAUGGAGGCGGUAAACAAGCCCUCGAAACCG |
| | UCCAGCGGCUUCUGCCGGUUCUGUGCCAGGCACAUGGCUUGACUC |
| | CUGAGCAAGUUGUCGCGAUUGCCUCCAACAUUGGGGGCAAGCAAG |
| | CACUCGAAACAGUGCAGAGGCUUCUACCAGUGCUCUGCCAGGCCC |
| | ACGGUUUGACCCCUGAACAGGUGGUCGCCAUCGCCUCCAACAACG |
| | GCGGAAAACAAGCACUGGAAACUGUGCAAAGACUGCUUCCUGUGC |
| | UGUGUCAGGCGCAUGGCCUCACCCCGGAACAAGUGGUGGCCAUAG |
| | CGUCGCACGACGGAGGCAAGCAGGCCCUUGAAACGGUGCAGCGAC |
| | UGUUGCCCGUCCUUUGUCAGGCUCACGGGUUAACCCCAGAACAGG |
| | UCGUGGCAAUCGCCUCUCAUGACGGGGGGAAGCAGGCCUUGGAGA |
| | CUGUCCAAAGGUUGCUGCCAGUGCUGUGCCAAGCCCACGGGCUCA |
| | CUCCAGAGCAAGUAGUGGCGAUAGCGAGCCACGACGGCGGGAAAC |
| | AGGCUCUGGAAACCGUACAGAGGUUGCUCCCCGUGCUUUGUCAAG |
| | CACAUGGUCUGACCCCCGAGCAAGUCGUGGCCAUUGCCAGCCAUG |
| | ACGGAGGGAAACAGGCACUUGAAACCGUCCAAAGGCUGCUGCCUG |
| | UGCUUUGCCAGGCGCAUGGGCUUACUCCUGAACAAGUGGUGGCGA |
| | UUGCUUCAAAUAUCGGUGGCAAACAAGCUUUGGAAACAGUCCAGC |
| | GGUUGCUCCCAGUCCUCUGUCAAGCUCACGGGCUGACGCCAGAAC |
| | AGGUCGUGGCAAUUGCGUCACACGACGGAGGAAAACAGGCGUUGG |
| | AAACCGUUCAGCGGCUCUUGCCCGUGCUAUGCCAGGCCCACGGAC |
| | UGACUCCGGAACAGGUGGUUGCCAUCGCCAGCAACAACGGAGGGA |
| | AGCAGGCGCUCGAGACUGUACAGCGACUUCUCCCCGUCCUGUGUC |
| | AAGCCCAUGGCCUGACGCCGGAACAGGUGGUAGCCAUUGCGAGCA |
| | ACGGAGGAGGACGCCCAGCCCUAGAGAGCAUCGUGGCCCAACUGU |
| | CGCGGCCGGAUCCAGCUCUGGCCGCAUUGACCAACGACCACCUGG |
| | UCGCUCUGGCUUGUCUGGGAGGACGACCGGCCCUGGAUGCCGUGA |
| | AGAAGGGCCUGCCACAUGCCCCUGCCCUCAUUAAGCGGACUAACA |
| | GAAGGAUCCCCGAGAGGACCUCACACAGAGUCGCUUCCGGAUCCG |
| | GUGGAGGAUCAGGCGGCGAUGCGAAGUCCCUCACCGCUUGGUCAC |
| | GGACCCUAGUGACCUUCAAAGACGUGUUCGUGGAUUUCACCAGGG |
| | AAGAAUGGAAGCUCCUGGACACCGCCCAGCAAAUACUGUACCGGA |
| | ACGUCAUGCUGGAGAACUAUAAGAACCUGGUCAGCUUGGGCUACC |
| | AGCUCACCAAGCCGGACGUGAUCCUGCGGCUGGAGAAGGGAGAGG |
| | AGCCUUGGCUCGUGGAAAGGGAAAUUCAUCAGGAGACUCACCCUG |
| | ACAGCGAAACCGCCUUCGAGAUUAAGUCCUCCGUGUCCGGAGGAA |
| | AGCGGCCCGCCGCCACCAAGAAGGCCGGCCAGGCCAAGAAGAAGA |
| | AGGGCAGCUACCCCUACGACGUGCCCGACUACGCCUGAGCGGCCG |
| | CUUAAUUAAGCUGCCUUCUGCGGGGCUUGCCUUCUGGCCAUGCCC |
| | UUCUUCUCUCCCUUGCACCUGUACCUCUUGGUCUUUGAAUAAAGC |
| | CUGAGUAGGAAGUCUAGAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAA |
| MR853 | AGGAAAUAAGAGAGAAAAGAAGAGUAAGAAGAAAUAUAAGAGCCA |
| TAL09- | CCAUGGCCCCCAAGAAGAAGCGGAAGGUGGGCAUCCACGGCGUGC |
| KRAB | CCGCCGCCGGCAGCAGCGGAUCCCACAUGGUGGACCUCCGGACCC |
| (SEQ ID NO: | UGGGAUACUCCCAGCAGCAGCAGGAAAAGAUCAAGCCGAAGGUCC |
| 455) | GCUCCACUGUGGCUCAACACCACGAAGCCCUUGUGGGACACGGCU |
| | UCACUCAUGCACACAUUGUGGCAUUGUCCCAACACCCUGCCGCUC |
| | UGGGAACUGUGGCAGUGAAGUACCAGGACAUGAUAGCCGCACUGC |
| | CCGAAGCAACACACGAGGCCAUCGUCGGUGUGGGAAAAAGAGGAG |
| | CGGGAGCCAGAGCACUCGAAGCGCUCCUGACAGUGGCGGGAGAAC |
| | UCCGCGGACCACCUCUGCAGCUGGACACUGGCCAGCUGCUGAAGA |
| | UUGCAAAACGCGGUGGAGUCACCGCUGUGGAGGCGGUGCAUGCCU |
| | GGAGAAAUGCCCUGACUGGUGCCCCCUUGAACCUGACCCCCGAGC |
| | AAGUCGUCGCAAUUGCUUCGAACAACGGUGGUAAACAGGCUCUCG |
| | AAACCGUCCAACGGCUGCUCCCAGUCCUAUGCCAAGCCCAUGGAC |
| | UUACGCCCGAACAAGUGGUUGCUAUAGCCUCCAAUAUCGGUGGGA |
| | AGCAGGCUCUGGAGACAGUCCAAAGACUCCUGCCGGUCCUCUGCC |
| | AAGCGCACGGACUCACCCCAGAGCAAGUAGUGGCCAUUGCCUCCC |
| | ACGAUGGUGGCAAGCAGGCCCUUGAAACAGUCCAGAGACUGUUAC |
| | CCGUGCUUUGUCAAGCCCACGGGCUCACUCCCGAACAGGUGGUGG |
| | CGAUUGCCUCUAACGGCGGCGGCAAACAGGCGCUCGAGACUGUCC |
| | AGCGGCUUCUACCGGUGUUGUGCCAGGCCCAUGGUCUGACACCCG |
| | AACAGGUGGUUGCAAUUGCCUCCAACGGAGGCGGGAAGCAAGCCC |
| | UCGAAACGGUCCAGCGGUUGUUGCCGGUCCUGUGUCAGGCGCACG |
| | GGUUGACCCCAGAACAGGUCGUCGCUAUUGCCUCAAACAUCGGUG |
| | GUAAGCAGGCGCUGGAAACUGUGCAGCGACUCCUGCCUGUGCUCU |
| | GUCAAGCGCACGGGCUUACGCCAGAACAAGUGGUCGCUAUCGCGU |
| | CCAACAACGGCGGCAAGCAAGCUCUGGAAACCGUCCAAAGGCUCU |
| | UGCCCGUCCUAUGUCAGGCCCACGGCCUCACCCCUGAACAGGUUG |
| | UCGCCAUCGCCAGCCACGACGGCGGUAAGCAAGCCUUGGAAACCG |
| | UGCAACGACUGCUUCCCGUGUUGUGUCAGGCUCAUGGCCUCACUC |
| | CUGAGCAAGUUGUGGCCAUCGCUUCGAAUAUUGGGGGCAAACAAG |
| | CCCUGGAAACGGUGCAGCGGUUGCUGCCUGUCUUGUGUCAAGCUC |
| | ACGGGCUGACUCCUGAACAAGUCGUGGCCAUAGCGUCCAACAUUG |
| | GGGGAAAGCAGGCGUUGGAAACUGUCCAGAGGCUGUUGCCCGUGC |
| | UCUGCCAGGCUCACGGCCUGACCCCUGAACAGGUGGUCGCAAUCG |
| | CCUCCAACAUCGGAGGGAAGCAGGCACUGGAAACAGUGCAGCGGC |
| | UGCUGCCCGUGCUGUGCCAAGCUCAUGGACUCACUCCGGAACAGG |
| | UCGUUGCAAUCGCAUCAAACAUUGGAGGAAAGCAGGCCCUCGAGA |
| | CAGUGCAGAGACUGCUCCCUGUGCUUUGCCAGGCGCAUGGCCUUA |
| | CCCCGGAACAAGUGGUAGCCAUCGCGUCGCAUGACGGCGGAAAGC |
| | AGGCUUUGGAAACCGUGCAAAGGCUCCUGCCCGUCCUGUGCCAGG |
| | CUCACGGUCUGACCCCGGAACAAGUCGUGGCCAUCGCUAGCCAUG |
| | AUGGAGGGAAACAGGCCCUGGAGACUGUACAGAGACUCCUCCCUG |
| | UCCUUUGCCAAGCACACGGACUUACCCCUGAACAGGUCGUGGCUA |
| | UUGCAUCGAACGGGGGGGGGAAACAAGCGCUCGAAACUGUGCAAC |
| | GCCUCCUGCCAGUGUUGUGCCAGGCACAUGGCUUGACUCCCGAGC |
| | AAGUCGUCGCCAUUGCAUCAAACAACGGAGGAAAACAGGCACUCG |
| | AGACUGUGCAAAGACUGUUGCCGGUGCUAUGCCAGGCACACGGCU |
| | UGACCCCGGAGCAAGUGGUGGCUAUCGCCUCGCACGAUGGCGGAA |
| | AACAAGCACUCGAAACUGUUCAGCGGCUACUGCCGGUGCUCUGCC |
| | AGGCUCACGGACUGACUCCAGAACAGGUGGUCGCAAUCGCGUCGA |
| | ACGGUGGAGGACGGCCUGCCCUCGAGAGCAUUGUGGCGCAGCUCU |
| | CCCGCCCGGAUCCUGCUUUGGCCGCGCUGACCAACGACCACCUGG |
| | UGGCCCUCGCCUGUUUGGGGGGUCGGCCUGCUUUGGACGCCGUCA |
| | AGAAGGGCCUGCCUCAUGCCCCCGCACUCAUCAAGCGGACCAAUA |
| | GACGCAUCCCGGAAAGGACUUCUCACCGGGUGGCUUCCGGAUCCG |
| | GUGGAGGAUCAGGCGGCGAUGCGAAGUCCCUCACCGCUUGGUCAC |
| | GGACCCUAGUGACCUUCAAAGACGUGUUCGUGGAUUUCACCAGGG |
| | AAGAAUGGAAGCUCCUGGACACCGCCCAGCAAAUACUGUACCGGA |
| | ACGUCAUGCUGGAGAACUAUAAGAACCUGGUCAGCUUGGGCUACC |
| | AGCUCACCAAGCCGGACGUGAUCCUGCGGCUGGAGAAGGGAGAGG |
| | AGCCUUGGCUCGUGGAAAGGGAAAUUCAUCAGGAGACUCACCCUG |
| | ACAGCGAAACCGCCUUCGAGAUUAAGUCCUCCGUGUCCGGAGGAA |
| | AGCGGCCCGCCGCCACCAAGAAGGCCGGCCAGGCCAAGAAGAAGA |
| | AGGGCAGCUACCCCUACGACGUGCCCGACUACGCCUGAGCGGCCG |
| | CUUAAUUAAGCUGCCUUCUGCGGGGCUUGCCUUCUGGCCAUGCCC |
| | UUCUUCUCUCCCUUGCACCUGUACCUCUUGGUCUUUGAAUAAAGC |
| | CUGAGUAGGAAGUCUAGAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAA |
| MR836 | AGGAAAUAAGAGAGAAAAGAAGAGUAAGAAGAAAUAUAAGAGCCA |
| TAL14- | CCAUGGCCCCCAAGAAGAAGCGGAAGGUGGGCAUCCACGGCGUGC |
| KRAB | CCGCCGCCGGCAGCAGCGGAUCCCACAUGGUCGAUCUUCGCACCC |
| (SEQ ID NO: | UCGGGUACUCGCAGCAGCAACAGGAAAAGAUCAAGCCGAAAGUCA |
| 456) | GGUCCACCGUCGCCCAGCACCACGAGGCACUGGUCGGCCACGGAU |
| | UCACCCACGCCCACAUCGUCGCGCUGUCUCAACAUCCAGCCGCCC |
| | UGGGAACUGUCGCCGUCAAGUACCAAGAUAUGAUCGCGGCACUGC |
| | CUGAAGCAACCCAUGAGGCCAUCGUGGGUGUGGGAAAGAGAGGAG |
| | CAGGGGCUAGAGCCCUGGAAGCGCUCCUGACCGUGGCGGGAGAGC |
| | UUAGAGGCCCUCCCCUGCAAUUGGACACCGGCCAACUUCUGAAGA |
| | UUGCAAAAAGGGGUGGAGUGACUGCCGUGGAAGCAGUGCACGCUU |
| | GGCGGAACGCACUGACUGGAGCACCCCUGAAUUUGACCCCCGAGC |
| | AAGUGGUUGCCAUCGCAAGCAACAUCGGAGGGAAACAAGCACUGG |
| | AAACUGUGCAGCGCCUGCUUCCGGUCCUGUGCCAAGCUCACGGAC |
| | UGACUCCUGAACAGGUUGUGGCCAUCGCGUCGCAUGACGGGGGAA |
| | AACAGGCGCUCGAAACCGUCCAAAGACUGCUCCCGGUGCUGUGUC |
| | AGGCUCACGGUUUGACCCCAGAACAAGUUGUCGCUAUCGCCUCAC |
| | ACGACGGCGGGAAGCAAGCUCUGGAGACUGUGCAAAGACUCCUUC |
| | CUGUGCUGUGCCAAGCUCAUGGAUUGACCCCGGAACAGGUCGUCG |
| | CCAUCGCUUCCAAUGGCGGAGGUAAACAGGCCUUGGAAACGGUGC |
| | AGAGGCUGCUGCCAGUCUUGUGUCAGGCGCACGGGCUGACUCCAG |
| | AGCAAGUAGUAGCCAUCGCCAGCAAUGGAGGCGGUAAACAAGCGC |
| | UCGAGACCGUCCAGCGCUUGUUGCCAGUGUUGUGCCAGGCCCAUG |
| | GGCUGACGCCCGAACAGGUGGUGGCCAUUGCAUCCAACAUUGGGG |
| | GGAAACAGGCGUUGGAGACAGUCCAACGGCUUCUGCCCGUCCUUU |
| | GUCAGGCACACGGACUCACUCCUGAACAAGUCGUUGCCAUUGCCU |
| | CGAAUAUCGGGGGUAAGCAAGCCCUCGAGACUGUGCAGAGAUUGU |
| | UGCCUGUCCUGUGUCAAGCGCAUGGUCUGACUCCCGAACAGGUGG |
| | UGGCGAUUGCUUCGAACAAUGGAGGAAAACAAGCAUUGGAAACCG |
| | UCCAGAGGCUGUUACCAGUCCUUUGCCAGGCACAUGGACUGACCC |
| | CGGAACAGGUCGUCGCAAUCGCAUCGAACAUUGGAGGCAAGCAGG |
| | CUCUCGAAACGGUCCAGAGACUGCUGCCUGUGCUCUGCCAAGCCC |
| | ACGGCCUCACUCCGGAGCAAGUCGUGGCAAUCGCCUCAAACAUCG |
| | GUGGAAAGCAGGCCCUUGAGACUGUCCAGCGACUGCUCCCAGUGC |
| | UCUGCCAGGCGCAUGGACUCACACCCGAGCAGGUGGUCGCCAUUG |
| | CUAGCAACAACGGAGGCAAACAAGCCCUGGAGACAGUGCAACGCU |
| | UGCUGCCCGUGCUUUGUCAAGCGCACGGACUUACUCCGGAACAAG |
| | UAGUGGCUAUUGCGAGUAGGCACGGCGGUAAGCAAGCCCUGGAGA |
| | CAGUGCAGCGGCUUUUGCCGGUGCUCUGUCAGGCCCACGGGCUCA |
| | CACCGGAACAGGUCGUAGCUAUUGCGUCCCAUGACGGCGGAAAGC |
| | AAGCCUUAGAAACUGUGCAGCGGUUGCUGCCUGUUCUGUGCCAGG |
| | CUCAUGGGCUCACUCCCGAGCAAGUGGUCGCGAUUGCGAGCAACG |
| | GAGGUGGCAAGCAAGCACUAGAAACCGUGCAACGACUGCUGCCGG |
| | UCUUGUGCCAAGCGCAUGGCCUGACACCCGAACAGGUGGUGGCUA |
| | UCGCGUCCAACAACGGGGGCAAACAGGCCCUGGAAACAGUCCAAA |
| | GGCUGUUGCCGGUCCUCUGUCAAGCUCACGGACUAACCCCCGAAC |
| | AGGUGGUGGCAAUAGCUUCGCGGCAUGGGGGCAAGCAGGCACUCG |
| | AAACAGUGCAGAGGUUGCUCCCGGUACUUUGCCAAGCACAUGGCU |
| | UGACCCCUGAGCAAGUCGUGGCCAUCGCAUCCAACGGCGGAGGGA |
| | AGCAAGCGCUUGAAACUGUCCAACGCCUCCUGCCCGUGUUGUGUC |
| | AAGCCCAUGGCCUUACGCCUGAACAAGUCGUCGCUAUUGCAAGUA |
| | ACGGAGGAGGGAGGCCUGCCCUGGAGUCCAUUGUCGCCCAACUGU |
| | CGCGACCCGAUCCAGCCCUGGCAGCCCUCACUAACGACCAUCUCG |
| | UGGCGCUGGCAUGCCUCGGUGGUAGACCAGCUCUGGACGCCGUGA |
| | AGAAGGGACUUCCUCAUGCCCCCGCACUGAUCAAGCGCACUAACC |
| | GGAGGAUCCCUGAAAGAACCUCCCACAGAGUGGCUUCCGGAUCCG |
| | GUGGAGGAUCAGGCGGCGAUGCGAAGUCCCUCACCGCUUGGUCAC |
| | GGACCCUAGUGACCUUCAAAGACGUGUUCGUGGAUUUCACCAGGG |
| | AAGAAUGGAAGCUCCUGGACACCGCCCAGCAAAUACUGUACCGGA |
| | ACGUCAUGCUGGAGAACUAUAAGAACCUGGUCAGCUUGGGCUACC |
| | AGCUCACCAAGCCGGACGUGAUCCUGCGGCUGGAGAAGGGAGAGG |
| | AGCCUUGGCUCGUGGAAAGGGAAAUUCAUCAGGAGACUCACCCUG |
| | ACAGCGAAACCGCCUUCGAGAUUAAGUCCUCCGUGUCCGGAGGAA |
| | AGCGGCCCGCCGCCACCAAGAAGGCCGGCCAGGCCAAGAAGAAGA |
| | AGGGCAGCUACCCCUACGACGUGCCCGACUACGCCUGAGCGGCCG |
| | CUUAAUUAAGCUGCCUUCUGCGGGGCUUGCCUUCUGGCCAUGCCC |
| | UUCUUCUCUCCCUUGCACCUGUACCUCUUGGUCUUUGAAUAAAGC |
| | CUGAGUAGGAAGUCUAGAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAA |
| MR942 | AGGAAAUAAGAGAGAAAAGAAGAGUAAGAAGAAAUAUAAGAGCCA |
| TAL27- | CCAUGGCCCCCAAGAAGAAGCGGAAGGUGGGCAUCCACGGCGUGC |
| KRAB mouse | CCGCCGCCGGCAGCAGCGGAUCCCACAUGGUGGACCUCCGCACGC |
| (SEQ ID NO: | UGGGAUACUCGCAACAGCAGCAGGAGAAGAUUAAGCCUAAGGUCC |
| 457) | GGUCCACCGUGGCUCAGCACCAUGAGGCGCUAGUGGGCCACGGAU |
| | UCACCCACGCGCAUAUUGUCGCCCUCUCGCAACAUCCCGCCGCAC |
| | UGGGAACCGUCGCCGUGAAGUACCAGGACAUGAUUGCCGCCCUGC |
| | CUGAAGCUACCCAUGAAGCCAUUGUGGGCGUGGGAAAGAGAGGAG |
| | CAGGGGCCAGAGCCUUGGAGGCCCUGCUCACGGUAGCCGGAGAGC |
| | UCAGAGGCCCACCACUGCAGCUUGACACCGGCCAACUUCUGAAGA |
| | UCGCCAAGCGGGGAGGAGUGACCGCCGUGGAGGCAGUGCAUGCCU |
| | GGCGCAACGCCUUGACUGGAGCCCCGCUGAACCUGACUCCGGAGC |
| | AAGUGGUGGCUAUCGCGUCCAACAUCGGAGGAAAACAGGCCCUGG |
| | AAACGGUGCAAAGACUGCUGCCGGUCCUCUGUCAAGCGCACGGAC |
| | UCACUCCCGAGCAAGUUGUGGCUAUCGCUUCCAACAUCGGGGGGA |
| | AACAGGCUCUGGAAACUGUCCAAAGGCUGUUACCUGUGCUGUGCC |
| | AAGCACACGGACUGACCCCAGAGCAAGUGGUCGCCAUAGCCUCCA |
| | ACAACGGGGGGAAGCAAGCCCUGGAGACUGUGCAGCGGUUGCUCC |
| | CCGUCCUGUGCCAGGCCCACGGAUUGACUCCCGAACAAGUGGUGG |
| | CCAUUGCGUCAAACAUCGGGGGCAAGCAAGCGUUGGAAACAGUCC |
| | AACGGUUGCUUCCCGUCCUUUGCCAGGCCCACGGUCUGACCCCGG |
| | AGCAAGUCGUCGCCAUUGCUAGUAACGGGGGAGGAAAGCAAGCUC |
| | UUGAAACCGUCCAGCGGCUUCUUCCGGUGCUCUGCCAAGCACACG |
| | GCCUCACACCCGAGCAAGUAGUGGCCAUUGCCUCAAACAUUGGGG |
| | GAAAGCAGGCAUUGGAAACUGUGCAACGGCUGCUUCCUGUCUUGU |
| | GCCAGGCGCAUGGGCUGACUCCAGAACAGGUGGUCGCUAUCGCGA |
| | GUAACAACGGUGGCAAACAGGCACUGGAAACCGUGCAGAGGCUCU |
| | UGCCUGUCCUGUGUCAGGCACACGGACUGACGCCAGAACAGGUGG |
| | UCGCUAUUGCGAGCAAUAUCGGCGGAAAGCAGGCGUUAGAAACUG |
| | UGCAGAGACUGCUCCCAGUGCUAUGCCAGGCUCAUGGGUUGACUC |
| | CUGAACAAGUGGUGGCUAUCGCCUCAAACGGUGGCGGUAAACAGG |
| | CGCUUGAGACAGUGCAAAGGCUCCUUCCCGUGUUGUGCCAAGCGC |
| | AUGGCCUGACCCCUGAACAGGUCGUGGCUAUCGCGUCAAACAACG |
| | GAGGCAAGCAGGCCCUAGAAACGGUACAACGGCUGCUGCCCGUAC |
| | UGUGCCAGGCCCACGGACUGACCCCAGAACAGGUGGUCGCAAUUG |
| | CCUCCAACAUUGGCGGGAAGCAGGCGCUAGAAACCGUGCAGCGGC |
| | UUCUGCCCGUCCUCUGCCAGGCCCACGGUCUGACUCCUGAGCAAG |
| | UCGUCGCAAUCGCCAGCAACAUCGGCGGAAAACAAGCCCUCGAAA |
| | CCGUCCAGCGACUGCUGCCUGUCCUAUGCCAGGCCCACGGGUUGA |
| | CCCCGGAACAGGUCGUCGCCAUUGCUUCCAAUAAUGGAGGCAAAC |
| | AGGCACUCGAAACGGUGCAGAGACUUCUGCCGGUGCUGUGUCAAG |
| | CUCACGGCUUGACCCCCGAGCAAGUGGUGGCAAUCGCUUCACGAC |
| | ACGGUGGAAAACAGGCCCUGGAGACAGUCCAGAGACUGCUCCCCG |
| | UGCUUUGCCAAGCUCACGGACUUACUCCGGAACAAGUCGUGGCGA |
| | UUGCGUCCAACGGUGGUGGAAAGCAGGCCUUGGAAACGGUCCAGA |
| | GACUGUUGCCGGUCUUGUGCCAGGCGCAUGGUCUGACCCCCGAAC |
| | AAGUCGUGGCAAUUGCAUCCCAUGACGGCGGGAAACAGGCUUUGG |
| | AGACUGUCCAGCGCCUGUUGCCAGUCCUUUGUCAAGCCCACGGGU |
| | UGACACCCGAACAGGUCGUCGCAAUCGCCUCGAACAUCGGUGGCA |
| | AGCAGGCCCUUGAAACUGUCCAACGCCUCCUCCCGGUGUUGUGUC |
| | AGGCUCACGGCCUGACUCCAGAACAAGUCGUGGCCAUAGCGUCAC |
| | ACGACGGUGGACGGCCAGCCCUGGAAUCCAUCGUGGCGCAGCUGU |
| | CCAGACCCGACCCAGCACUGGCGGCCCUCACUAACGAUCACCUUG |
| | UGGCCCUGGCCUGUCUGGGAGGUAGACCAGCCCUCGACGCUGUGA |
| | AGAAGGGACUGCCGCAUGCACCAGCCCUGAUCAAGCGAACCAACC |
| | GGCGCAUUCCCGAGAGAACUUCACACAGAGUGGCUAGCGGCAGCG |
| | GCGGCGGCAGCGGCGGCGACGCCAAGAGCCUGACCGCCUGGAGCC |
| | GGACCCUGGUGACCUUCAAGGACGUGUUCGUGGACUUCACCCGGG |
| | AGGAGUGGAAGCUGCUGGACACCGCCCAGCAGAUCCUGUACCGGA |
| | ACGUGAUGCUGGAGAACUACAAGAACCUGGUGAGCCUGGGCUACC |
| | AGCUGACCAAGCCCGACGUGAUCCUGCGGCUGGAGAAGGGCGAGG |
| | AGCCCUGGCUGGUGGAGCGGGAGAUCCACCAGGAAACCCACCCCG |
| | ACAGCGAAACCGCCUUCGAGAUCAAGAGCAGCGUGAGCGGCGGCA |
| | AGCGGCCCGCCGCCACCAAGAAGGCCGGCCAGGCCAAGAAGAAGA |
| | AGGGCAGCUACCCCUACGACGUGCCCGACUACGCCUGAGCGGCCG |
| | CUUAAUUAAGCUGCCUUCUGCGGGGCUUGCCUUCUGGCCAUGCCC |
| | UUCUUCUCUCCCUUGCACCUGUACCUCUUGGUCUUUGAAUAAAGC |
| | CUGAGUAGGAAGUCUAGAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAA |
| MR968 | GGAAAUAAGAGAGAAAAGAAGAGUAAGAAGAAAUAUAAGAGCCAC |
| ZF05-KRAB- | CAUGGCCCCAAAGAAGAAGAGAAAGGUCGGAAUUCAUGGCGUGCC |
| tPT2A- | CGCAGCCGGCAGCAGCGGUUCCCUGGAGCCCGGAGAGAAACCGUA |
| TAL14- | UAAGUGCCCGGAAUGUGGAAAGUCCUUCUCCCAAUCCGGCCACCU |
| KRAB | GACUGAGCAUCAGCGCACCCACACUGGCGAAAAGCCGUACAAGUG |
| (SEQ ID NO: | CCCUGAGUGCGGGAAGUCGUUUAGCCAGCGGGCGAACUUGCGCGC |
| 458) | UCAUCAGCGGACUCAUACCGGGGAGAAGCCCUAUAAAUGCCCGGA |
| | GUGCGGAAAGAGCUUCAGCACCAAGAACUCCCUGACUGAACACCA |
| | GCGGACCCACACCGGAGAGAAGCCUUACAAGUGUCCUGAAUGCGG |
| | GAAAUCCUUCUCCACCUCCGGUUCACUCGUGCGCCACCAGAGAAC |
| | GCACACCGGUGAAAAGCCUUAUAAGUGUCCAGAGUGCGGCAAAAG |
| | CUUCAGCCAGUCCGGCAACCUCACCGAGCACCAACGGACUCACAC |
| | UGGAGAAAAGCCCUACAAAUGUCCGGAAUGCGGCAAAUCAUUCUC |
| | GCAAGCCGGUCACCUUGCCUCGCACCAGCGCACACACACCGGGGA |
| | AAAGCCAUACAAAUGCCCCGAAUGCGGAAAGUCGUUCUCUCAAUC |
| | CGGCGACCUGAGAAGGCAUCAGAGGACCCAUACCGGAAAGAAAAC |
| | CUCCGCCAGCGGAUCAGGAGGUGGAUCAGGCGGAGAUGCCAAGUC |
| | ACUGACCGCAUGGUCCCGGACCCUUGUGACCUUCAAGGACGUGUU |
| | CGUGGACUUCACCAGAGAGGAGUGGAAGCUGCUGGACACCGCGCA |
| | ACAGAUUCUCUACCGGAACGUGAUGCUGGAGAACUACAAGAAUCU |
| | GGUGUCCCUGGGCUACCAGCUGACCAAGCCGGACGUGAUCCUCCG |
| | CCUCGAAAAGGGGGAGGAGCCUUGGCUCGUGGAGCGGGAAAUCCA |
| | CCAGGAGACUCACCCGGACUCCGAGACUGCCUUCGAAAUCAAGUC |
| | AUCGGUGUCCGGUGGAAAGCGGCCUGCAGCGACUAAGAAGGCCGG |
| | ACAGGCCAAGAAGAAGAAGGGCUCAGGCUCAGCAACCAACUUCUC |
| | CUUGCUCAAACAAGCCGGGGACGUCGAAGAGAACCCCGGACCGAC |
| | UAGCGCCGGAAAGCUGGGUUCCGGAGAGGGUCGAGGUUCCCUCCU |
| | UACCUGUGGAGAUGUGGAAGAAAACCCAGGACCCCUGGAGGGCUC |
| | CAGCGGUAGCGGAAGCCCGAAGAAGAAGCGGAAGGUCGGAAUCCA |
| | CGGAGUGCCUGCCGCUGGAAGCUCGGGUUCCCACAUGGUCGAUCU |
| | UCGCACCCUCGGGUACUCGCAGCAGCAACAGGAAAAGAUCAAGCC |
| | GAAAGUCAGGUCCACCGUCGCCCAGCACCACGAGGCACUGGUCGG |
| | CCACGGAUUCACCCACGCCCACAUCGUCGCGCUGUCUCAACAUCC |
| | AGCCGCCCUGGGAACUGUCGCCGUCAAGUACCAAGAUAUGAUCGC |
| | GGCACUGCCUGAAGCAACCCAUGAGGCCAUCGUGGGUGUGGGAAA |
| | GAGAGGAGCAGGGGCUAGAGCCCUGGAAGCGCUCCUGACCGUGGC |
| | GGGAGAGCUUAGAGGCCCUCCCCUGCAAUUGGACACCGGCCAACU |
| | UCUGAAGAUUGCAAAAAGGGGUGGAGUGACUGCCGUGGAAGCAGU |
| | GCACGCUUGGCGGAACGCACUGACUGGAGCACCCCUGAAUUUGAC |
| | CCCCGAGCAAGUGGUUGCCAUCGCAAGCAACAUCGGAGGGAAACA |
| | AGCACUGGAAACUGUGCAGCGCCUGCUUCCGGUCCUGUGCCAAGC |
| | UCACGGACUGACUCCUGAACAGGUUGUGGCCAUCGCGUCGCAUGA |
| | CGGGGGAAAACAGGCGCUCGAAACCGUCCAAAGACUGCUCCCGGU |
| | GCUGUGUCAGGCUCACGGUUUGACCCCAGAACAAGUUGUCGCUAU |
| | CGCCUCACACGACGGCGGGAAGCAAGCUCUGGAGACUGUGCAAAG |
| | ACUCCUUCCUGUGCUGUGCCAAGCUCAUGGAUUGACCCCGGAACA |
| | GGUCGUCGCCAUCGCUUCCAAUGGCGGAGGUAAACAGGCCUUGGA |
| | AACGGUGCAGAGGCUGCUGCCAGUCUUGUGUCAGGCGCACGGGCU |
| | GACUCCAGAGCAAGUAGUAGCCAUCGCCAGCAAUGGAGGCGGUAA |
| | ACAAGCGCUCGAGACCGUCCAGCGCUUGUUGCCAGUGUUGUGCCA |
| | GGCCCAUGGGCUGACGCCCGAACAGGUGGUGGCCAUUGCAUCCAA |
| | CAUUGGGGGGAAACAGGCGUUGGAGACAGUCCAACGGCUUCUGCC |
| | CGUCCUUUGUCAGGCACACGGACUCACUCCUGAACAAGUCGUUGC |
| | CAUUGCCUCGAAUAUCGGGGGUAAGCAAGCCCUCGAGACUGUGCA |
| | GAGAUUGUUGCCUGUCCUGUGUCAAGCGCAUGGUCUGACUCCCGA |
| | ACAGGUGGUGGCGAUUGCUUCGAACAAUGGAGGAAAACAAGCAUU |
| | GGAAACCGUCCAGAGGCUGUUACCAGUCCUUUGCCAGGCACAUGG |
| | ACUGACCCCGGAACAGGUCGUCGCAAUCGCAUCGAACAUUGGAGG |
| | CAAGCAGGCUCUCGAAACGGUCCAGAGACUGCUGCCUGUGCUCUG |
| | CCAAGCCCACGGCCUCACUCCGGAGCAAGUCGUGGCAAUCGCCUC |
| | AAACAUCGGUGGAAAGCAGGCCCUUGAGACUGUCCAGCGACUGCU |
| | CCCAGUGCUCUGCCAGGCGCAUGGACUCACACCCGAGCAGGUGGU |
| | CGCCAUUGCUAGCAACAACGGAGGCAAACAAGCCCUGGAGACAGU |
| | GCAACGCUUGCUGCCCGUGCUUUGUCAAGCGCACGGACUUACUCC |
| | GGAACAAGUAGUGGCUAUUGCGAGUAGGCACGGCGGUAAGCAAGC |
| | CCUGGAGACAGUGCAGCGGCUUUUGCCGGUGCUCUGUCAGGCCCA |
| | CGGGCUCACACCGGAACAGGUCGUAGCUAUUGCGUCCCAUGACGG |
| | CGGAAAGCAAGCCUUAGAAACUGUGCAGCGGUUGCUGCCUGUUCU |
| | GUGCCAGGCUCAUGGGCUCACUCCCGAGCAAGUGGUCGCGAUUGC |
| | GAGCAACGGAGGUGGCAAGCAAGCACUAGAAACCGUGCAACGACU |
| | GCUGCCGGUCUUGUGCCAAGCGCAUGGCCUGACACCCGAACAGGU |
| | GGUGGCUAUCGCGUCCAACAACGGGGGCAAACAGGCCCUGGAAAC |
| | AGUCCAAAGGCUGUUGCCGGUCCUCUGUCAAGCUCACGGACUAAC |
| | CCCCGAACAGGUGGUGGCAAUAGCUUCGCGGCAUGGGGGCAAGCA |
| | GGCACUCGAAACAGUGCAGAGGUUGCUCCCGGUACUUUGCCAAGC |
| | ACAUGGCUUGACCCCUGAGCAAGUCGUGGCCAUCGCAUCCAACGG |
| | CGGAGGGAAGCAAGCGCUUGAAACUGUCCAACGCCUCCUGCCCGU |
| | GUUGUGUCAAGCCCAUGGCCUUACGCCUGAACAAGUCGUCGCUAU |
| | UGCAAGUAACGGAGGAGGGAGGCCUGCCCUGGAGUCCAUUGUCGC |
| | CCAACUGUCGCGACCCGAUCCAGCCCUGGCAGCCCUCACUAACGA |
| | CCAUCUCGUGGCGCUGGCAUGCCUCGGUGGUAGACCAGCUCUGGA |
| | CGCCGUGAAGAAGGGACUUCCUCAUGCCCCCGCACUGAUCAAGCG |
| | CACUAACCGGAGGAUCCCUGAAAGAACCUCCCACAGAGUGGCAUC |
| | CGGUUCAGGAGGAGGGUCCGGAGGAGCUAGGGAUGAUGCCAAGAG |
| | CUUGACCGCCUGGUCACGGACUCUUGUCACUUUCAAGGAUGUCUU |
| | UGUGGACUUUACCAGAGAAGAAUGGAAGCUCCUGGACACUGCGCA |
| | GCAGAUCCUGUACAGAAACGUGAUGUUGGAAAACUACAAAAACCU |
| | GGUGUCACUCGGGUACCAACUCACCAAGCCCGACGUCAUCCUGCG |
| | CCUGGAAAAGGGCGAAGAACCGUGGUUGGUCGAGAGAGAAAUCCA |
| | UCAGGAAACCCACCCUGACUCGGAAACCGCCUUCGAGAUCAAGUC |
| | CUCGGUGCCUAGCUCCGGAGGAAAAAGGCCCGCCGCAACCAAGAA |
| | GGCUGGGCAGGCUAAGAAAAAGAAGGGGUCCGGCUCCUAAUGAGC |
| | GGCCGCUUAAUUAAGCUGCCUUCUGCGGGGCUUGCCUUCUGGCCA |
| | UGCCCUUCUUCUCUCCCUUGCACCUGUACCUCUUGGUCUUUGAAU |
| | AAAGCCUGAGUAGGAAGUCUAGAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAA |
| MR1048 | GGAAAUAAGAGAGAAAAGAAGAGUAAGAAGAAAUAUAAGAGCCAC |
| TAL37- | CAUGGCCCCAAAGAAGAAGCGGAAAGUCGGAGGUUCAGGCGGCUC |
| KRAB mouse | GCACAUGGUGGACCUGAGAACUCUGGGUUACUCCCAGCAACAGCA |
| (SEQ ID NO: | AGAAAAAAUCAAGCCAAAAGUGCGCAGCACUGUCGCGCAGCACCA |
| 459) | CGAGGCCCUUGUGGGGCACGGGUUCACCCACGCUCACAUCGUGGC |
| | CCUGAGCCAGCAUCCGGCAGCCCUGGGAACCGUCGCAGUGAAGUA |
| | CCAAGACAUGAUCGCCGCCUUGCCGGAAGCCACUCACGAAGCAAU |
| | UGUGGGAGUGGGGAAGAGAGGAGCCGGCGCAAGAGCCCUCGAAGC |
| | GCUUCUGACUGUGGCCGGAGAAUUGAGAGGCCCCCCACUGCAGCU |
| | GGAUACCGGACAACUGCUGAAGAUCGCCAAACGGGGUGGAGUGAC |
| | CGCCGUGGAAGCGGUGCACGCGUGGAGAAACGCGCUGACUGGAGC |
| | CCCGCUCAACCUGACCCCUGAACAGGUCGUGGCUAUUGCCUCAAA |
| | CAACGGCGGGAAGCAAGCGUUGGAGACUGUACAGCGCCUGCUGCC |
| | AGUGUUGUGUCAAGCCCACGGGCUGACUCCCGAGCAAGUGGUGGC |
| | CAUAGCCAGCAAUAUCGGAGGUAAACAGGCUCUCGAGACUGUCCA |
| | ACGCCUGCUCCCAGUGCUUUGUCAGGCUCACGGCCUCACUCCUGA |
| | ACAGGUCGUAGCCAUUGCCAGCAACGGUGGCGGCAAACAAGCCUU |
| | AGAGACUGUGCAACGGCUCUUGCCGGUGCUCUGUCAGGCCCACGG |
| | ACUGACCCCAGAACAAGUAGUCGCUAUCGCGUCAAACAUCGGCGG |
| | CAAGCAGGCUUUGGAAACCGUCCAGCGGCUUCUGCCGGUGCUGUG |
| | CCAGGCGCAUGGGCUCACCCCCGAACAGGUGGUCGCAAUCGCCUC |
| | GAACGGCGGUGGUAAACAAGCCCUCGAAACUGUCCAGCGCCUCUU |
| | GCCCGUCCUGUGUCAGGCCCAUGGCCUGACUCCAGAACAGGUGGU |
| | GGCAAUAGCGUCCCAUGACGGGGGAAAGCAAGCCCUUGAGACUGU |
| | GCAACGGUUGCUCCCCGUGUUAUGUCAGGCACAUGGUCUGACCCC |
| | CGAACAGGUGGUCGCAAUUGCGUCGAACGGCGGAGGAAAGCAAGC |
| | UCUGGAAACGGUCCAACGGCUGCUCCCUGUGCUGUGCCAGGCGCA |
| | CGGACUUACCCCAGAACAAGUGGUCGCCAUUGCGUCCAACAUCGG |
| | AGGGAAGCAGGCGCUUGAAACCGUGCAGAGGUUGCUGCCGGUGUU |
| | AUGCCAAGCACAUGGACUAACCCCGGAGCAAGUGGUGGCCAUUGC |
| | CUCGAAUAUUGGGGGGAAACAAGCGUUAGAAACUGUGCAGAGGCU |
| | GCUGCCUGUGCUCUGCCAAGCCCAUGGUCUUACCCCGGAACAGGU |
| | UGUGGCGAUUGCAUCCAACAACGGUGGAAAACAGGCCCUUGAAAC |
| | GGUGCAGCGGCUCCUUCCUGUCCUGUGCCAAGCCCAUGGACUCAC |
| | CCCUGAACAGGUCGUGGCUAUUGCCUCCAACGGAGGCGGCAAGCA |
| | GGCCCUGGAGACAGUCCAGAGACUGCUGCCCGUGCUGUGUCAAGC |
| | UCACGGUCUGACUCCGGAACAGGUGGUUGCUAUCGCUUCGCACGA |
| | CGGCGGAAAACAAGCUCUUGAAACCGUGCAAAGGCUGCUCCCGGU |
| | GCUUUGCCAAGCUCAUGGCUUGACCCCAGAGCAAGUCGUGGCAAU |
| | CGCGUCCAAUAUCGGUGGGAAGCAGGCUCUUGAGACAGUGCAAAG |
| | ACUCCUCCCCGUCCUCUGUCAAGCGCACGGGCUCACUCCUGAACA |
| | GGUCGUCGCUAUUGCUUCGAACAACGGGGGCAAACAGGCACUAGA |
| | GACUGUCCAAAGACUUUUACCUGUCCUCUGCCAGGCACACGGAUU |
| | GACUCCCGAACAAGUCGUCGCGAUUGCCUCAAGACACGGGGGCAA |
| | GCAAGCGCUGGAGACUGUUCAGCGACUGCUUCCUGUGCUUUGCCA |
| | GGCCCACGGUUUGACCCCUGAGCAAGUUGUGGCCAUCGCCUCUAA |
| | CGGAGGUGGAAAGCAGGCCUUGGAAACAGUGCAGAGACUGUUGCC |
| | CGUCUUGUGCCAGGCUCAUGGGCUUACGCCUGAACAAGUCGUCGC |
| | CAUCGCUUCGAACGGAGGAGGGAAACAGGCGCUGGAAACUGUGCA |
| | GCGACUCCUUCCGGUCCUUUGUCAAGCACACGGCCUUACGCCGGA |
| | ACAAGUGGUUGCCAUCGCAAGCCACGACGGAGGACGCCCUGCCCU |
| | CGAGUCCAUUGUGGCCCAGCUGUCACGCCCAGAUCCUGCACUGGC |
| | CGCUCUUACCAACGAUCACCUGGUCGCUCUCGCGUGCCUGGGAGG |
| | AAGGCCUGCCCUGGAUGCCGUCAAGAAGGGACUGCCUCAUGCCCC |
| | UGCUUUGAUUAAGAGGACUAAUCGGCGCAUCCCCGAACGGACCAG |
| | CCACAGGGUGGCAUCUGGGAGCGGAGGAGGAUCCGGCGGAGACGC |
| | CAAGAGCCUGACCGCCUGGAGCCGGACCCUGGUGACCUUCAAGGA |
| | CGUGUUCGUGGACUUCACCCGGGAGGAGUGGAAGCUGCUGGACAC |
| | CGCCCAGCAGAUCCUGUACCGGAACGUGAUGCUGGAGAACUACAA |
| | GAACCUGGUGAGCCUGGGCUACCAGCUGACCAAGCCCGACGUGAU |
| | CCUGCGGCUGGAGAAGGGCGAGGAGCCCUGGCUGGUGGAGCGGGA |
| | GAUCCACCAGGAAACCCACCCCGACAGCGAAACCGCCUUCGAGAU |
| | CAAGAGCAGCGUGAGCGGCGGCAAGCGCCCAGCGGCUACAAAGAA |
| | GGCCGGACAGGCCAAGAAGAAGAAGGGUUCGUACCCCUACGACGU |
| | GCCUGACUAUGCCUGAGCGGCCGCUUAAUUAAGCUGCCUUCUGCG |
| | GGGCUUGCCUUCUGGCCAUGCCCUUCUUCUCUCCCUUGCACCUGU |
| | ACCUCUUGGUCUUUGAAUAAAGCCUGAGUAGGAAGUCUAGAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAA |
| MR1045 | GGAAAUAAGAGAGAAAAGAAGAGUAAGAAGAAAUAUAAGAGCCAC |
| TAL34- | CAUGGCCCCAAAGAAGAAGCGGAAAGUCGGAGGUUCAGGCGGCUC |
| KRAB mouse | GCACAUGGUGGACCUGAGAACUCUGGGUUACUCCCAGCAACAGCA |
| (SEQ ID NO: | AGAAAAAAUCAAGCCAAAAGUGCGCAGCACUGUCGCGCAGCACCA |
| 460) | CGAGGCCCUUGUGGGGCACGGGUUCACCCACGCUCACAUCGUGGC |
| | CCUGAGCCAGCAUCCGGCAGCCCUGGGAACCGUCGCAGUGAAGUA |
| | CCAAGACAUGAUCGCCGCCUUGCCGGAAGCCACUCACGAAGCAAU |
| | UGUGGGAGUGGGGAAGAGAGGAGCCGGCGCAAGAGCCCUCGAAGC |
| | GCUUCUGACUGUGGCCGGAGAAUUGAGAGGCCCCCCACUGCAGCU |
| | GGAUACCGGACAACUGCUGAAGAUCGCCAAACGGGGUGGAGUGAC |
| | UGCCGUCGAGGCUGUGCACGCCUGGAGAAACGCCUUGACCGGAGC |
| | GCCACUGAACCUUACCCCGGAGCAAGUGGUGGCGAUAGCAUCAAA |
| | UAUUGGCGGAAAGCAGGCCCUGGAAACCGUACAAAGACUGUUGCC |
| | GGUCCUGUGUCAAGCUCAUGGUUUGACCCCAGAACAGGUCGUGGC |
| | UAUUGCAUCGAACGGCGGAGGCAAACAAGCCCUCGAGACAGUCCA |
| | GAGACUUCUGCCGGUUCUGUGCCAGGCGCACGGACUUACCCCUGA |
| | ACAGGUGGUGGCGAUUGCGAGCCACGACGGUGGUAAACAGGCGUU |
| | GGAAACUGUGCAGCGGUUGCUUCCGGUGCUGUGUCAGGCUCACGG |
| | GCUGACCCCCGAACAAGUGGUAGCUAUCGCCUCCCACGACGGAGG |
| | GAAGCAGGCUCUGGAAACGGUGCAACGCCUCCUUCCCGUCUUGUG |
| | CCAAGCACAUGGACUGACUCCAGAACAGGUCGUCGCCAUUGCCUC |
| | CCAUGAUGGGGGGAAGCAGGCACUGGAAACUGUACAACGCCUGCU |
| | GCCUGUCCUCUGCCAAGCGCACGGUUUGACGCCGGAACAGGUUGU |
| | CGCCAUCGCCUCGAACGGAGGGGGAAAACAGGCCCUUGAAACAGU |
| | GCAACGGCUGCUGCCAGUGCUGUGUCAGGCGCAUGGCUUGACCCC |
| | GGAGCAAGUGGUGGCCAUAGCCUCGAAUGGAGGAGGAAAGCAGGC |
| | CCUGGAAACCGUUCAGCGCCUCCUCCCUGUGCUGUGUCAGGCCCA |
| | UGGGCUCACUCCCGAACAGGUAGUCGCCAUAGCGUCGAACAUCGG |
| | AGGAAAACAAGCGCUCGAAACUGUCCAAAGACUGCUUCCAGUCCU |
| | UUGCCAGGCUCAUGGACUAACUCCAGAGCAAGUAGUGGCCAUCGC |
| | GAGCCAUGACGGCGGCAAGCAAGCACUCGAAACCGUGCAGAGGCU |
| | GCUUCCCGUCCUGUGCCAAGCUCACGGCCUGACGCCUGAACAGGU |
| | GGUGGCUAUCGCCAGCAACGGUGGUGGAAAGCAGGCGCUCGAGAC |
| | UGUGCAGAGGUUGCUCCCAGUGCUCUGUCAGGCACAUGGCCUCAC |
| | CCCGGAGCAAGUGGUGGCAAUCGCCUCAAACAUCGGUGGCAAACA |
| | GGCACUUGAAACCGUGCAGCGCCUUUUGCCUGUGCUCUGCCAGGC |
| | CCACGGACUGACCCCUGAGCAAGUCGUGGCUAUUGCCUCUAACGG |
| | CGGUGGCAAGCAGGCCUUGGAAACCGUCCAGAGGCUCUUGCCGGU |
| | GCUCUGCCAGGCACACGGACUCACUCCGGAACAGGUGGUCGCCAU |
| | CGCAUCGAAUAUCGGCGGAAAACAGGCCCUCGAAACGGUCCAGCG |
| | GUUACUCCCCGUGCUUUGUCAGGCUCACGGAUUGACUCCCGAGCA |
| | AGUCGUGGCCAUUGCUUCCAACAUUGGGGGGAAACAGGCGCUGGA |
| | AACGGUGCAGCGACUCCUUCCUGUGUUGUGUCAAGCCCACGGCCU |
| | CACUCCAGAGCAAGUCGUGGCAAUUGCCUCCAACAUCGGGGGAAA |
| | GCAAGCCUUGGAGACAGUACAGCGGCUGCUUCCUGUGCUUUGCCA |
| | AGCCCAUGGACUCACCCCCGAACAAGUGGUUGCCAUUGCCAGCCA |
| | CGAUGGAGGGAAACAAGCCCUGGAGACAGUGCAAAGGCUCCUGCC |
| | CGUGCUGUGCCAGGCACACGGCUUGACUCCUGAACAAGUCGUCGC |
| | UAUCGCUUCCAAUGGUGGUGGGAAGCAAGCUCUGGAGACUGUCCA |
| | GCGGCUCCUGCCAGUGUUGUGUCAAGCACACGGUCUGACCCCGGA |
| | ACAAGUCGUUGCAAUCGCGUCGCAUGAUGGAGGACGCCCUGCCCU |
| | CGAGUCCAUUGUGGCCCAGCUGUCACGCCCAGAUCCUGCACUGGC |
| | CGCUCUUACCAACGAUCACCUGGUCGCUCUCGCGUGCCUGGGAGG |
| | AAGGCCUGCCCUGGAUGCCGUCAAGAAGGGACUGCCUCAUGCCCC |
| | UGCUUUGAUUAAGAGGACUAAUCGGCGCAUCCCCGAACGGACCAG |
| | CCACAGGGUGGCAUCUGGGAGCGGAGGAGGAUCCGGCGGAGACGC |
| | CAAGAGCCUGACCGCCUGGAGCCGGACCCUGGUGACCUUCAAGGA |
| | CGUGUUCGUGGACUUCACCCGGGAGGAGUGGAAGCUGCUGGACAC |
| | CGCCCAGCAGAUCCUGUACCGGAACGUGAUGCUGGAGAACUACAA |
| | GAACCUGGUGAGCCUGGGCUACCAGCUGACCAAGCCCGACGUGAU |
| | CCUGCGGCUGGAGAAGGGCGAGGAGCCCUGGCUGGUGGAGCGGGA |
| | GAUCCACCAGGAAACCCACCCCGACAGCGAAACCGCCUUCGAGAU |
| | CAAGAGCAGCGUGAGCGGCGGCAAGCGCCCAGCGGCUACAAAGAA |
| | GGCCGGACAGGCCAAGAAGAAGAAGGGUUCGUACCCCUACGACGU |
| | GCCUGACUAUGCCUGAGCGGCCGCUUAAUUAAGCUGCCUUCUGCG |
| | GGGCUUGCCUUCUGGCCAUGCCCUUCUUCUCUCCCUUGCACCUGU |
| | ACCUCUUGGUCUUUGAAUAAAGCCUGAGUAGGAAGUCUAGAAAAA |
| | AAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAA |
| | AAAAAAAAAAAAAAA |

TABLE 14
Exemplary Sequences: Epigenetic Modifying
Agents Amino Acid

| Name | Amino Acid Sequence |
|---|---|
| OEC695 | MAPKKKRKVGIHGVPAAGSSGSLEPGEKPYKCPECGKSFS |
| ZF07-KRAB | DKKDLTRHQRTHTGEKPYKCPECGKSFSQSSSLVRHQRTH |
| (SEQ ID NO: | TGEKPYKCPECGKSFSTTGNLTVHQRTHTGEKPYKCPECG |
| 461) | KSFSTSGELVRHQRTHTGEKPYKCPECGKSFSTTGNLTVH |
| | QRTHTGEKPYKCPECGKSFSTSHSLTEHQRTHTGEKPYKC |
| | PECGKSFSTSGNLVRHQRTHTGKKTSASGSGGGSGGDAKS |
| | LTAWSRTLVTFKDVFVDFTREEWKLLDTAQQILYRNVMLE |
| | NYKNLVSLGYQLTKPDVILRLEKGEEPWLVEREIHQETHP |
| | DSETAFEIKSSVSGGKRPAATKKAGQAKKKKGSYPYDVPD |
| | YA |
| OEC693 | MAPKKKRKVGIHGVPAAGSSGSHMVDLRTLGYSQQQQEKI |
| TAL27- | KPKVRSTVAQHHEALVGHGFTHAHIVALSQHPAALGTVAV |
| KRAB | KYQDMIAALPEATHEAIVGVGKRGAGARALEALLTVAGEL |
| (SEQ ID NO: | RGPPLQLDTGQLLKIAKRGGVTAVEAVHAWRNALTGAPLN |
| 462) | LTPEQVVAIASNIGGKQALETVQRLLPVLCQAHGLTPEQV |
| | VAIASNIGGKQALETVQRLLPVLCQAHGLTPEQVVAIASN |
| | NGGKQALETVQRLLPVLCQAHGLTPEQVVAIASNIGGKQA |
| | LETVQRLLPVLCQAHGLTPEQVVAIASNGGGKQALETVQR |
| | LLPVLCQAHGLTPEQVVAIASNIGGKQALETVQRLLPVLC |
| | QAHGLTPEQVVAIASNNGGKQALETVQRLLPVLCQAHGLT |
| | PEQVVAIASNIGGKQALETVQRLLPVLCQAHGLTPEQVVA |
| | IASNGGGKQALETVQRLLPVLCQAHGLTPEQVVAIASNNG |
| | GKQALETVQRLLPVLCQAHGLTPEQVVAIASNIGGKQALE |
| | TVQRLLPVLCQAHGLTPEQVVAIASNIGGKQALETVQRLL |
| | PVLCQAHGLTPEQVVAIASNNGGKQALETVQRLLPVLCQA |
| | HGLTPEQVVAIASRHGGKQALETVQRLLPVLCQAHGLTPE |
| | QVVAIASNGGGKQALETVQRLLPVLCQAHGLTPEQVVAIA |
| | SHDGGKQALETVQRLLPVLCQAHGLTPEQVVAIASNIGGK |
| | QALETVQRLLPVLCQAHGLTPEQVVAIASHDGGRPALESI |
| | VAQLSRPDPALAALTNDHLVALACLGGRPALDAVKKGLPH |
| | APALIKRTNRRIPERTSHRVASGSGGGSGGDAKSLTAWSR |
| | TLVTFKDVFVDFTREEWKLLDTAQQILYRNVMLENYKNLV |
| | SLGYQLTKPDVILRLEKGEEPWLVEREIHQETHPDSETAF |
| | EIKSSVSGGKRPAATKKAGQAKKKKGSYPYDVPDYA |
| OEC151 | MAPKKKRKVGIHGVPAAGSSGSLEPGEKPYKCPECGKSFS |
| ZF24-KRAB | SKKHLAEHQRTHTGEKPYKCPECGKSFSQNSTLTEHQRTH |
| (SEQ ID NO: | TGEKPYKCPECGKSFSQSGDLRRHQRTHTGEKPYKCPECG |
| 463) | KSFSTHLDLIRHQRTHTGEKPYKCPECGKSFSDPGNLVRH |
| | QRTHTGEKPYKCPECGKSFSTSGNLTEHQRTHTGEKPYKC |
| | PECGKSFSERSHLREHQRTHTGKKTSASGSGGGSGGDAKS |
| | LTAWSRTLVTFKDVFVDFTREEWKLLDTAQQILYRNVMLE |
| | NYKNLVSLGYQLTKPDVILRLEKGEEPWLVEREIHQETHP |
| | DSETAFEIKSSVSGGKRPAATKKAGQAKKKKGSYPYDVPD |
| | YA |
| OEC144 | MAPKKKRKVGIHGVPAAGSSGSLEPGEKPYKCPECGKSFS |
| ZF16-KRAB | QRAHLERHQRTHTGEKPYKCPECGKSFSRSDHLTNHQRTH |
| (SEQ ID NO: | TGEKPYKCPECGKSFSRNDALTEHQRTHTGEKPYKCPECG |
| 464) | KSFSRADNLTEHQRTHTGEKPYKCPECGKSFSSKKALTEH |
| | QRTHTGEKPYKCPECGKSFSRNDALTEHQRTHTGEKPYKC |
| | PECGKSFSHKNALQNHQRTHTGKKTSASGSGGGSGGDAKS |
| | LTAWSRTLVTFKDVFVDFTREEWKLLDTAQQILYRNVMLE |
| | NYKNLVSLGYQLTKPDVILRLEKGEEPWLVEREIHQETHP |
| | DSETAFEIKSSVSGGKRPAATKKAGQAKKKKGSYPYDVPD |
| | YA |
| OEC134 | MAPKKKRKVGIHGVPAAGSSGSLEPGEKPYKCPECGKSFS |
| ZF07-MQ1 | DKKDLTRHQRTHTGEKPYKCPECGKSFSQSSSLVRHQRTH |
| (SEQ ID NO: | TGEKPYKCPECGKSFSTTGNLTVHQRTHTGEKPYKCPECG |
| 465) | KSFSTSGELVRHQRTHTGEKPYKCPECGKSFSTTGNLTVH |
| | QRTHTGEKPYKCPECGKSFSTSHSLTEHQRTHTGEKPYKC |
| | PECGKSFSTSGNLVRHQRTHTGKKTSASGSGGGSGGARDS |
| | KVENKTKKLRVFEAFAGIGAQRKALEKVRKDEYEIVGLAE |
| | WYVPAIVMYQAIHNNFHTKLEYKSVSREEMIDYLENKTLS |
| | WNSKNPVSNGYWKRKKDDELKIIYNAIKLSEKEGNIFDIR |
| | DLYKRTLKNIDLLTYSFPCQDLSQQGIQKGMKRGSGTRSG |
| | LLWEIERALDSTEKNDLPKYLLMENVGALLHKKNEEELNQ |
| | WKQKLESLGYQNSIEVLNAADFGSSQARRRVFMISTLNEF |
| | VELPKGDKKPKSIKKVLNKIVSEKDILNNLLKYNLTEFKK |
| | TKSNINKASLIGYSKFNSEGYVYDPEFTGPTLTASGANSR |
| | IKIKDGSNIRKMNSDETFLYIGFDSQDGKRVNEIEFLTEN |
| | QKIFVCGNSISVEVLEAIIDKIGGPSSGGKRPAATKKAGQ |
| | AKKKKGSYPYDVPDYA |
| OEC129 | MAPKKKRKVGIHGVPAAGSSGSLEPGEKPYKCPECGKSFS |
| ZF05-MQ1 | QSGHLTEHQRTHTGEKPYKCPECGKSFSQRANLRAHQRTH |
| (SEQ ID NO: | TGEKPYKCPECGKSFSTKNSLTEHQRTHTGEKPYKCPECG |
| 466) | KSFSTSGSLVRHQRTHTGEKPYKCPECGKSFSQSGNLTEH |
| | QRTHTGEKPYKCPECGKSFSQAGHLASHQRTHTGEKPYKC |
| | PECGKSFSQSGDLRRHQRTHTGKKTSASGSGGGSGGARDS |
| | KVENKTKKLRVFEAFAGIGAQRKALEKVRKDEYEIVGLAE |
| | WYVPAIVMYQAIHNNFHTKLEYKSVSREEMIDYLENKTLS |
| | WNSKNPVSNGYWKRKKDDELKIIYNAIKLSEKEGNIFDIR |
| | DLYKRTLKNIDLLTYSFPCQDLSQQGIQKGMKRGSGTRSG |
| | LLWEIERALDSTEKNDLPKYLLMENVGALLHKKNEEELNQ |
| | WKQKLESLGYQNSIEVLNAADFGSSQARRRVFMISTLNEF |
| | VELPKGDKKPKSIKKVLNKIVSEKDILNNLLKYNLTEFKK |
| | TKSNINKASLIGYSKFNSEGYVYDPEFTGPTLTASGANSR |
| | IKIKDGSNIRKMNSDETFLYIGFDSQDGKRVNEIEFLTEN |
| | QKIFVCGNSISVEVLEAIIDKIGGPSSGGKRPAATKKAGQ |
| | AKKKKGSYPYDVPDYA |
| OEC126 | MAPKKKRKVGIHGVPAAGSSGSLEPGEKPYKCPECGKSFS |
| ZF05-KRAB | QSGHLTEHQRTHTGEKPYKCPECGKSFSQRANLRAHQRTH |
| (SEQ ID NO: | TGEKPYKCPECGKSFSTKNSLTEHQRTHTGEKPYKCPECG |
| 467) | KSFSTSGSLVRHQRTHTGEKPYKCPECGKSFSQSGNLTEH |
| | QRTHTGEKPYKCPECGKSFSQAGHLASHQRTHTGEKPYKC |
| | PECGKSFSQSGDLRRHQRTHTGKKTSASGSGGGSGGDAKS |
| | LTAWSRTLVTFKDVFVDFTREEWKLLDTAQQILYRNVMLE |
| | NYKNLVSLGYQLTKPDVILRLEKGEEPWLVEREIHQETHP |
| | DSETAFEIKSSVSGGKRPAATKKAGQAKKKKGSYPYDVPD |
| | YA |
| OEC103 | MAPKKKRKVGIHGVPAAGSSGSHMVDLRTLGYSQQQQEKI |
| TAL14- | KPKVRSTVAQHHEALVGHGFTHAHIVALSQHPAALGTVAV |
| KRAB | KYQDMIAALPEATHEAIVGVGKRGAGARALEALLTVAGEL |
| (SEQ ID NO: | RGPPLQLDTGQLLKIAKRGGVTAVEAVHAWRNALTGAPLN |
| 468) | LTPEQVVAIASNIGGKQALETVQRLLPVLCQAHGLTPEQV |
| | VAIASHDGGKQALETVQRLLPVLCQAHGLTPEQVVAIASH |
| | DGGKQALETVQRLLPVLCQAHGLTPEQVVAIASNGGGKQA |
| | LETVQRLLPVLCQAHGLTPEQVVAIASNGGGKQALETVQR |
| | LLPVLCQAHGLTPEQVVAIASNIGGKQALETVQRLLPVLC |
| | QAHGLTPEQVVAIASNIGGKQALETVQRLLPVLCQAHGLT |
| | PEQVVAIASNNGGKQALETVQRLLPVLCQAHGLTPEQVVA |
| | IASNIGGKQALETVQRLLPVLCQAHGLTPEQVVAIASNIG |
| | GKQALETVQRLLPVLCQAHGLTPEQVVAIASNNGGKQALE |
| | TVQRLLPVLCQAHGLTPEQVVAIASRHGGKQALETVQRLL |
| | PVLCQAHGLTPEQVVAIASHDGGKQALETVQRLLPVLCQA |
| | HGLTPEQVVAIASNGGGKQALETVQRLLPVLCQAHGLTPE |
| | QVVAIASNNGGKQALETVQRLLPVLCQAHGLTPEQVVAIA |
| | SRHGGKQALETVQRLLPVLCQAHGLTPEQVVAIASNGGGK |
| | QALETVQRLLPVLCQAHGLTPEQVVAIASNGGGRPALESI |
| | VAQLSRPDPALAALTNDHLVALACLGGRPALDAVKKGLPH |
| | APALIKRTNRRIPERTSHRVASGSGGGSGGDAKSLTAWSR |
| | TLVTFKDVFVDFTREEWKLLDTAQQILYRNVMLENYKNLV |
| | SLGYQLTKPDVILRLEKGEEPWLVEREIHQETHPDSETAF |
| | EIKSSVSGGKRPAATKKAGQAKKKKGSYPYDVPDYA |
| OEC099 | MAPKKKRKVGIHGVPAAGSSGSHMVDLRTLGYSQQQQEKI |
| TAL09- | KPKVRSTVAQHHEALVGHGFTHAHIVALSQHPAALGTVAV |
| KRAB | KYQDMIAALPEATHEAIVGVGKRGAGARALEALLTVAGEL |
| (SEQ ID NO: | RGPPLQLDTGQLLKIAKRGGVTAVEAVHAWRNALTGAPLN |
| 469) | LTPEQVVAIASNNGGKQALETVQRLLPVLCQAHGLTPEQV |
| | VAIASNIGGKQALETVQRLLPVLCQAHGLTPEQVVAIASH |
| | DGGKQALETVQRLLPVLCQAHGLTPEQVVAIASNGGGKQA |
| | LETVQRLLPVLCQAHGLTPEQVVAIASNGGGKQALETVQR |
| | LLPVLCQAHGLTPEQVVAIASNIGGKQALETVQRLLPVLC |
| | QAHGLTPEQVVAIASNNGGKQALETVQRLLPVLCQAHGLT |
| | PEQVVAIASHDGGKQALETVQRLLPVLCQAHGLTPEQVVA |
| | IASNIGGKQALETVQRLLPVLCQAHGLTPEQVVAIASNIG |
| | GKQALETVQRLLPVLCQAHGLTPEQVVAIASNIGGKQALE |
| | TVQRLLPVLCQAHGLTPEQVVAIASNIGGKQALETVQRLL |
| | PVLCQAHGLTPEQVVAIASHDGGKQALETVQRLLPVLCQA |
| | HGLTPEQVVAIASHDGGKQALETVQRLLPVLCQAHGLTPE |
| | QVVAIASNGGGKQALETVQRLLPVLCQAHGLTPEQVVAIA |
| | SNNGGKQALETVQRLLPVLCQAHGLTPEQVVAIASHDGGK |
| | QALETVQRLLPVLCQAHGLTPEQVVAIASNGGGRPALESI |
| | VAQLSRPDPALAALTNDHLVALACLGGRPALDAVKKGLPH |
| | APALIKRTNRRIPERTSHRVASGSGGGSGGDAKSLTAWSR |
| | TLVTFKDVFVDFTREEWKLLDTAQQILYRNVMLENYKNLV |
| | SLGYQLTKPDVILRLEKGEEPWLVEREIHQETHPDSETAF |
| | EIKSSVSGGKRPAATKKAGQAKKKKGSYPYDVPDYA |
| OEC095 | MAPKKKRKVGIHGVPAAGSSGSHMVDLRTLGYSQQQQEKI |
| TAL08- | KPKVRSTVAQHHEALVGHGFTHAHIVALSQHPAALGTVAV |
| KRAB | KYQDMIAALPEATHEAIVGVGKRGAGARALEALLTVAGEL |
| (SEQ ID NO: | RGPPLQLDTGQLLKIAKRGGVTAVEAVHAWRNALTGAPLN |
| 470) | LTPEQVVAIASNNGGKQALETVQRLLPVLCQAHGLTPEQV |
| | VAIASRHGGKQALETVQRLLPVLCQAHGLTPEQVVAIASH |
| | DGGKQALETVQRLLPVLCQAHGLTPEQVVAIASNIGGKQA |
| | LETVQRLLPVLCQAHGLTPEQVVAIASHDGGKQALETVQR |
| | LLPVLCQAHGLTPEQVVAIASNIGGKQALETVQRLLPVLC |
| | QAHGLTPEQVVAIASHDGGKQALETVQRLLPVLCQAHGLT |
| | PEQVVAIASNGGGKQALETVQRLLPVLCQAHGLTPEQVVA |
| | IASNIGGKQALETVQRLLPVLCQAHGLTPEQVVAIASNNG |
| | GKQALETVQRLLPVLCQAHGLTPEQVVAIASHDGGKQALE |
| | TVQRLLPVLCQAHGLTPEQVVAIASHDGGKQALETVQRLL |
| | PVLCQAHGLTPEQVVAIASHDGGKQALETVQRLLPVLCQA |
| | HGLTPEQVVAIASHDGGKQALETVQRLLPVLCQAHGLTPE |
| | QVVAIASNIGGKQALETVQRLLPVLCQAHGLTPEQVVAIA |
| | SHDGGKQALETVQRLLPVLCQAHGLTPEQVVAIASNNGGK |
| | QALETVQRLLPVLCQAHGLTPEQVVAIASNGGGRPALESI |
| | VAQLSRPDPALAALTNDHLVALACLGGRPALDAVKKGLPH |
| | APALIKRTNRRIPERTSHRVASGSGGGSGGDAKSLTAWSR |
| | TLVTFKDVFVDFTREEWKLLDTAQQILYRNVMLENYKNLV |
| | SLGYQLTKPDVILRLEKGEEPWLVEREIHQETHPDSETAF |
| | EIKSSVSGGKRPAATKKAGQAKKKKGSYPYDVPDYA |
| OEC087 | MAPKKKRKVGIHGVPAAGSSGSHMVDLRTLGYSQQQQEKI |
| TAL04- | KPKVRSTVAQHHEALVGHGFTHAHIVALSQHPAALGTVAV |
| KRAB | KYQDMIAALPEATHEAIVGVGKRGAGARALEALLTVAGEL |
| (SEQ ID NO: | RGPPLQLDTGQLLKIAKRGGVTAVEAVHAWRNALTGAPLN |
| 471) | LTPEQVVAIASNNGGKQALETVQRLLPVLCQAHGLTPEQV |
| | VAIASHDGGKQALETVQRLLPVLCQAHGLTPEQVVAIASN |
| | IGGKQALETVQRLLPVLCQAHGLTPEQVVAIASNIGGKQA |
| | LETVQRLLPVLCQAHGLTPEQVVAIASHDGGKQALETVQR |
| | LLPVLCQAHGLTPEQVVAIASNNGGKQALETVQRLLPVLC |
| | QAHGLTPEQVVAIASNIGGKQALETVQRLLPVLCQAHGLT |
| | PEQVVAIASNGGGKQALETVQRLLPVLCQAHGLTPEQVVA |
| | IASHDGGKQALETVQRLLPVLCQAHGLTPEQVVAIASNIG |
| | GKQALETVQRLLPVLCQAHGLTPEQVVAIASNIGGKQALE |
| | TVQRLLPVLCQAHGLTPEQVVAIASNGGGKQALETVQRLL |
| | PVLCQAHGLTPEQVVAIASNNGGKQALETVQRLLPVLCQA |
| | HGLTPEQVVAIASRHGGKQALETVQRLLPVLCQAHGLTPE |
| | QVVAIASRHGGKQALETVQRLLPVLCQAHGLTPEQVVAIA |
| | SNIGGKQALETVQRLLPVLCQAHGLTPEQVVAIASNGGGK |
| | QALETVQRLLPVLCQAHGLTPEQVVAIASNGGGRPALESI |
| | VAQLSRPDPALAALTNDHLVALACLGGRPALDAVKKGLPH |
| | APALIKRTNRRIPERTSHRVASGSGGGSGGDAKSLTAWSR |
| | TLVTFKDVFVDFTREEWKLLDTAQQILYRNVMLENYKNLV |
| | SLGYQLTKPDVILRLEKGEEPWLVEREIHQETHPDSETAF |
| | EIKSSVSGGKRPAATKKAGQAKKKKGSYPYDVPDYA |
| OEC056 | MAPKKKRKVGGSGGSHMVDLRTLGYSQQQQEKIKPKVRST |
| TAL37- | VAQHHEALVGHGFTHAHIVALSQHPAALGTVAVKYQDMIA |
| KRAB | ALPEATHEAIVGVGKRGAGARALEALLTVAGELRGPPLQL |
| (SEQ ID NO: | DTGQLLKIAKRGGVTAVEAVHAWRNALTGAPLNLTPEQVV |
| 472) | AIASNNGGKQALETVQRLLPVLCQAHGLTPEQVVAIASNI |
| | GGKQALETVQRLLPVLCQAHGLTPEQVVAIASNGGGKQAL |
| | ETVQRLLPVLCQAHGLTPEQVVAIASNIGGKQALETVQRL |
| | LPVLCQAHGLTPEQVVAIASNGGGKQALETVQRLLPVLCQ |
| | AHGLTPEQVVAIASHDGGKQALETVQRLLPVLCQAHGLTP |
| | EQVVAIASNGGGKQALETVQRLLPVLCQAHGLTPEQVVAI |
| | ASNIGGKQALETVQRLLPVLCQAHGLTPEQVVAIASNIGG |
| | KQALETVQRLLPVLCQAHGLTPEQVVAIASNNGGKQALET |
| | VQRLLPVLCQAHGLTPEQVVAIASNGGGKQALETVQRLLP |
| | VLCQAHGLTPEQVVAIASHDGGKQALETVQRLLPVLCQAH |
| | GLTPEQVVAIASNIGGKQALETVQRLLPVLCQAHGLTPEQ |
| | VVAIASNNGGKQALETVQRLLPVLCQAHGLTPEQVVAIAS |
| | RHGGKQALETVQRLLPVLCQAHGLTPEQVVAIASNGGGKQ |
| | ALETVQRLLPVLCQAHGLTPEQVVAIASNGGGKQALETVQ |
| | RLLPVLCQAHGLTPEQVVAIASHDGGRPALESIVAQLSRP |
| | DPALAALTNDHLVALACLGGRPALDAVKKGLPHAPALIKR |
| | TNRRIPERTSHRVASGSGGGSGGDAKSLTAWSRTLVTFKD |
| | VFVDFTREEWKLLDTAQQILYRNVMLENYKNLVSLGYQLT |
| | KPDVILRLEKGEEPWLVEREIHQETHPDSETAFEIKSSVS |
| | GGKRPAATKKAGQAKKKKGSYPYDVPDYA |
| OEC053 | MAPKKKRKVGGSGGSHMVDLRTLGYSQQQQEKIKPKVRST |
| TAL34- | VAQHHEALVGHGFTHAHIVALSQHPAALGTVAVKYQDMIA |
| KRAB | ALPEATHEAIVGVGKRGAGARALEALLTVAGELRGPPLQL |
| (SEQ ID NO: | DTGQLLKIAKRGGVTAVEAVHAWRNALTGAPLNLTPEQVV |
| 473) | AIASNIGGKQALETVQRLLPVLCQAHGLTPEQVVAIASNG |
| | GGKQALETVQRLLPVLCQAHGLTPEQVVAIASHDGGKQAL |
| | ETVQRLLPVLCQAHGLTPEQVVAIASHDGGKQALETVQRL |
| | LPVLCQAHGLTPEQVVAIASHDGGKQALETVQRLLPVLCQ |
| | AHGLTPEQVVAIASNGGGKQALETVQRLLPVLCQAHGLTP |
| | EQVVAIASNGGGKQALETVQRLLPVLCQAHGLTPEQVVAI |
| | ASNIGGKQALETVQRLLPVLCQAHGLTPEQVVAIASHDGG |
| | KQALETVQRLLPVLCQAHGLTPEQVVAIASNGGGKQALET |
| | VQRLLPVLCQAHGLTPEQVVAIASNIGGKQALETVQRLLP |
| | VLCQAHGLTPEQVVAIASNGGGKQALETVQRLLPVLCQAH |
| | GLTPEQVVAIASNIGGKQALETVQRLLPVLCQAHGLTPEQ |
| | VVAIASNIGGKQALETVQRLLPVLCQAHGLTPEQVVAIAS |
| | NIGGKQALETVQRLLPVLCQAHGLTPEQVVAIASHDGGKQ |
| | ALETVQRLLPVLCQAHGLTPEQVVAIASNGGGKQALETVQ |
| | RLLPVLCQAHGLTPEQVVAIASHDGGRPALESIVAQLSRP |
| | DPALAALTNDHLVALACLGGRPALDAVKKGLPHAPALIKR |
| | TNRRIPERTSHRVASGSGGGSGGDAKSLTAWSRTLVTFKD |
| | VFVDFTREEWKLLDTAQQILYRNVMLENYKNLVSLGYQLT |
| | KPDVILRLEKGEEPWLVEREIHQETHPDSETAFEIKSSVS |
| | GGKRPAATKKAGQAKKKKGSYPYDVPDYA |
| OEC043 | MAPKKKRKVGIHGVPAAGSSGSLEPGEKPYKCPECGKSFS |
| ZF05-KRAB- | QSGHLTEHQRTHTGEKPYKCPECGKSFSQRANLRAHQRTH |
| tPT2A- | TGEKPYKCPECGKSFSTKNSLTEHQRTHTGEKPYKCPECG |
| TAL14- | KSFSTSGSLVRHQRTHTGEKPYKCPECGKSFSQSGNLTEH |
| KRAB | QRTHTGEKPYKCPECGKSFSQAGHLASHQRTHTGEKPYKC |
| (SEQ ID NO: | PECGKSFSQSGDLRRHQRTHTGKKTSASGSGGGSGGDAKS |
| 474) | LTAWSRTLVTFKDVFVDFTREEWKLLDTAQQILYRNVMLE |
| | NYKNLVSLGYQLTKPDVILRLEKGEEPWLVEREIHQETHP |
| | DSETAFEIKSSVSGGKRPAATKKAGQAKKKKGSGSATNFS |
| | LLKQAGDVEENPGPTSAGKLGSGEGRGSLLTCGDVEENPG |
| | PLEGSSGSGSPKKKRKVGIHGVPAAGSSGSHMVDLRTLGY |
| | SQQQQEKIKPKVRSTVAQHHEALVGHGFTHAHIVALSQHP |
| | AALGTVAVKYQDMIAALPEATHEAIVGVGKRGAGARALEA |
| | LLTVAGELRGPPLQLDTGQLLKIAKRGGVTAVEAVHAWRN |
| | ALTGAPLNLTPEQVVAIASNIGGKQALETVQRLLPVLCQA |
| | HGLTPEQVVAIASHDGGKQALETVQRLLPVLCQAHGLTPE |
| | QVVAIASHDGGKQALETVQRLLPVLCQAHGLTPEQVVAIA |
| | SNGGGKQALETVQRLLPVLCQAHGLTPEQVVAIASNGGGK |
| | QALETVQRLLPVLCQAHGLTPEQVVAIASNIGGKQALETV |
| | QRLLPVLCQAHGLTPEQVVAIASNIGGKQALETVQRLLPV |
| | LCQAHGLTPEQVVAIASNNGGKQALETVQRLLPVLCQAHG |
| | LTPEQVVAIASNIGGKQALETVQRLLPVLCQAHGLTPEQV |
| | VAIASNIGGKQALETVQRLLPVLCQAHGLTPEQVVAIASN |
| | NGGKQALETVQRLLPVLCQAHGLTPEQVVAIASRHGGKQA |
| | LETVQRLLPVLCQAHGLTPEQVVAIASHDGGKQALETVQR |
| | LLPVLCQAHGLTPEQVVAIASNGGGKQALETVQRLLPVLC |
| | QAHGLTPEQVVAIASNNGGKQALETVQRLLPVLCQAHGLT |
| | PEQVVAIASRHGGKQALETVQRLLPVLCQAHGLTPEQVVA |
| | IASNGGGKQALETVQRLLPVLCQAHGLTPEQVVAIASNGG |
| | GRPALESIVAQLSRPDPALAALTNDHLVALACLGGRPALD |
| | AVKKGLPHAPALIKRTNRRIPERTSHRVASGSGGGSGGAR |
| | DDAKSLTAWSRTLVTFKDVFVDFTREEWKLLDTAQQILYR |
| | NVMLENYKNLVSLGYQLTKPDVILRLEKGEEPWLVEREIH |
| | QETHPDSETAFEIKSSVPSSGGKRPAATKKAGQAKKKKGS |
| | GS |

### Exemplary Sequences

>dCas9 amino acid sequence
SEQ ID NO.: 299
DKKYSIGLAIGTNSVGWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRIPYYVG
PLARGNSRFAWMTRKSEETITPWNFEEVVDKGASAQSFIERMTNFDKNLPNEKVLPKHSLLYEYF
TVYNELTKVKYVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNSR
ERMKRIEEGIKELGSQILKEHPVENTQLQNEKLYLYYLQNGRDMYVDQELDINRLSDYDVAAIVP
QSFLKDDSIDNKVLTRSDKARGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLS
QLGGD
>dCas9 mRNA sequence
SEQ ID NO.: 300
GACAAGAAGUACAGCAUCGGCCUGGCCAUCGGCACCAACAGCGUGGGCUGGGCCGUGAUCACCGA
CGAGUACAAGGUGCCCAGCAAGAAGUUCAAGGUGCUGGGCAACACCGACCGGCACAGCAUCAAGA
AGAACCUGAUCGGCGCCCUGCUGUUCGACAGCGGCGAGACCGCCGAGGCCACCCGGCUGAAGCGG
ACCGCCCGGCGGCGGUACACCCGGCGGAAGAACCGGAUCUGCUACCUGCAGGAGAUCUUCAGCAA
CGAGAUGGCCAAGGUGGACGACAGCUUCUUCCACCGGCUGGAGGAGAGCUUCCUGGUGGAGGAGG
ACAAGAAGCACGAGCGGCACCCCAUCUUCGGCAACAUCGUGGACGAGGUGGCCUACCACGAGAAG
UACCCCACCAUCUACCACCUGCGGAAGAAGCUGGUGGACAGCACCGACAAGGCCGACCUGCGGCU
GAUCUACCUGGCCCUGGCCCACAUGAUCAAGUUCCGGGGCCACUUCCUGAUCGAGGGCGACCUGA
ACCCCGACAACAGCGACGUGGACAAGCUGUUCAUCCAGCUGGUGCAGACCUACAACCAGCUGUUC
GAGGAGAACCCCAUCAACGCCAGCGGCGUGGACGCCAAGGCCAUCCUGAGCGCCCGGCUGAGCAA
GAGCCGGCGGCUGGAGAACCUGAUCGCCCAGCUGCCCGGCGAGAAGAAGAACGGCCUGUUCGGCA
ACCUGAUCGCCCUGAGCCUGGGCCUGACCCCCAACUUCAAGAGCAACUUCGACCUGGCCGAGGAC
GCCAAGCUGCAGCUGAGCAAGGACACCUACGACGACGACCUGGACAACCUGCUGGCCCAGAUCGG
CGACCAGUACGCCGACCUGUUCCUGGCCGCCAAGAACCUGAGCGACGCCAUCCUGCUGAGCGACA
UCCUGCGGGUGAACACCGAGAUCACCAAGGCCCCCCUGAGCGCCAGCAUGAUCAAGCGGUACGAC
GAGCACCACCAGGACCUGACCCUGCUGAAGGCCCUGGUGCGGCAGCAGCUGCCCGAGAAGUACAA
GGAGAUCUUCUUCGACCAGAGCAAGAACGGCUACGCCGGCUACAUCGACGGCGGCGCCAGCCAGG
AGGAGUUCUACAAGUUCAUCAAGCCCAUCCUGGAGAAGAUGGACGGCACCGAGGAGCUGCUGGUG
AAGCUGAACCGGGAGGACCUGCUGCGGAAGCAGCGGACCUUCGACAACGGCAGCAUCCCCCACCA
GAUCCACCUGGGCGAGCUGCACGCCAUCCUGCGGCGGCAGGAGGACUUCUACCCCUUCCUGAAGG
ACAACCGGGAGAAGAUCGAGAAGAUCCUGACCUUCCGGAUCCCCUACUACGUGGGCCCCCUGGCC
CGGGGCAACAGCCGGUUCGCCUGGAUGACCCGGAAAUCCGAGGAGACCAUCACCCCCUGGAACUU
CGAGGAGGUGGUGGACAAGGGCGCCAGCGCCCAGAGCUUCAUCGAGCGGAUGACCAACUUCGACA
AGAACCUGCCCAACGAGAAGGUGCUGCCCAAGCACAGCCUGCUGUACGAGUACUUCACCGUGUAC
AACGAGCUGACCAAGGUGAAGUACGUGACCGAGGGCAUGCGGAAGCCCGCCUUCCUGAGCGGCGA
GCAGAAGAAGGCCAUCGUGGACCUGCUGUUCAAGACCAACCGGAAGGUGACCGUGAAGCAGCUGA
AGGAGGACUACUUCAAGAAGAUCGAGUGCUUCGACAGCGUGGAGAUCAGCGGCGUGGAGGACCGG
UUCAACGCCAGCCUGGGCACCUACCACGACCUGCUGAAGAUCAUCAAGGACAAGGACUUCCUGGA
CAACGAGGAGAACGAGGACAUCCUGGAGGACAUCGUGCUGACCCUGACCCUGUUCGAGGACCGGG
AGAUGAUCGAGGAGCGGCUGAAAACCUACGCCCACCUGUUCGACGACAAGGUGAUGAAGCAGCUG
AAGCGGCGGCGGUACACCGGCUGGGGCCGGCUGAGCCGGAAGCUGAUCAACGGCAUCCGGGACAA
GCAGAGCGGCAAGACCAUCCUGGACUUCCUGAAAUCCGACGGCUUCGCCAACCGGAACUUCAUGC
AGCUGAUCCACGACGACAGCCUGACCUUCAAGGAGGACAUCCAGAAGGCCCAGGUGAGCGGCCAG
GGCGACAGCCUGCACGAGCACAUCGCCAACCUGGCCGGCAGCCCCGCCAUCAAGAAGGGCAUCCU
GCAGACCGUGAAGGUGGUGGACGAGCUGGUGAAGGUGAUGGGCCGGCACAAGCCCGAGAACAUCG
UGAUCGAGAUGGCCCGGGAGAACCAGACCACCCAGAAGGGCCAGAAGAACAGCCGGGAGCGGAUG
AAGCGGAUCGAGGAGGGCAUCAAGGAGCUGGGCAGCCAGAUCCUGAAGGAGCACCCCGUGGAGAA
CACCCAGCUGCAGAACGAGAAGCUGUACCUGUACUACCUGCAGAACGGCCGGGACAUGUACGUGG
ACCAGGAGCUGGACAUCAACCGGCUGAGCGACUACGACGUGGCCGCCAUCGUGCCCCAGAGCUUC
CUGAAGGACGACAGCAUCGACAACAAGGUGCUGACCCGGAGCGACAAGGCCCGGGGCAAGAGCGA
CAACGUGCCCAGCGAGGAGGUGGUGAAGAAGAUGAAGAACUACUGGCGGCAGCUGCUGAACGCCA
AGCUGAUCACCCAGCGGAAGUUCGACAACCUGACCAAGGCCGAGCGGGGCGGCCUGAGCGAGCUG
GACAAGGCCGGCUUCAUCAAGCGGCAGCUGGUGGAGACCCGGCAGAUCACCAAGCACGUGGCCCA
GAUCCUGGACAGCCGGAUGAACACCAAGUACGACGAGAACGACAAGCUGAUCCGGGAGGUGAAGG
UGAUCACCCUGAAAUCCAAGCUGGUGAGCGACUUCCGGAAGGACUUCCAGUUCUACAAGGUGCGG
GAGAUCAACAACUACCACCACGCCCACGACGCCUACCUGAACGCCGUGGUGGGCACCGCCCUGAU
CAAGAAGUACCCCAAGCUGGAGAGCGAGUUCGUGUACGGCGACUACAAGGUGUACGACGUGCGGA
AGAUGAUCGCCAAGAGCGAGCAGGAGAUCGGCAAGGCCACCGCCAAGUACUUCUUCUACAGCAAC
AUCAUGAACUUCUUCAAGACCGAGAUCACCCUGGCCAACGGCGAGAUCCGGAAGCGGCCCCUGAU
CGAGACCAACGGCGAGACCGGCGAGAUCGUGUGGGACAAGGGCCGGGACUUCGCCACCGUGCGGA
AGGUGCUGAGCAUGCCCCAGGUGAACAUCGUGAAGAAAACCGAGGUGCAGACCGGCGGCUUCAGC
AAGGAGAGCAUCCUGCCCAAGCGGAACAGCGACAAGCUGAUCGCCCGGAAGAAGGACUGGGACCC
CAAGAAGUACGGCGGCUUCGACAGCCCCACCGUGGCCUACAGCGUGCUGGUGGUGGCCAAGGUGG
AGAAGGGCAAGAGCAAGAAGCUGAAAUCCGUGAAGGAGCUGCUGGGCAUCACCAUCAUGGAGCGG
AGCAGCUUCGAGAAGAACCCCAUCGACUUCCUGGAGGCCAAGGGCUACAAGGAGGUGAAGAAGGA
CCUGAUCAUCAAGCUGCCCAAGUACAGCCUGUUCGAGCUGGAGAACGGCCGGAAGCGGAUGCUGG
CCAGCGCCGGCGAGCUGCAGAAGGGCAACGAGCUGGCCCUGCCCAGCAAGUACGUGAACUUCCUG
UACCUGGCCAGCCACUACGAGAAGCUGAAGGGCAGCCCCGAGGACAACGAGCAGAAGCAGCUGUU
CGUGGAGCAGCACAAGCACUACCUGGACGAGAUCAUCGAGCAGAUCAGCGAGUUCAGCAAGCGGG
UGAUCCUGGCCGACGCCAACCUGGACAAGGUGCUGAGCGCCUACAACAAGCACCGGGACAAGCCC
AUCCGGGAGCAGGCCGAGAACAUCAUCCACCUGUUCACCCUGACCAACCUGGGCGCCCCCGCCGC
CUUCAAGUACUUCGACACCACCAUCGACCGGAAGCGGUACACCAGCACCAAGGAGGUGCUGGACG
CCACCCUGAUCCACCAGAGCAUCACCGGCCUGUACGAGACCCGGAUCGACCUGAGCCAGCUGGGC
GGCGAC
>Amino acid sequence of the Effector Fusion
Protein of dCas9-MQ1
SEQ ID NO: 159
MAPKKKRKVGIHGVPAADKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQELD
INRLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIH
QSITGLYETRIDLSQLGGDKRPAATKKAGQAKKKKARDSKVENKTKKLRVFEAFAGIGAQRKALE
KVRKDEYEIVGLAEWYVPAIVMYQAIHNNFHTKLEYKSVSREEMIDYLENKTLSWNSKNPVSNGY
WKRKKDDELKIIYNAIKLSEKEGNIFDIRDLYKRTLKNIDLLTYSFPCQDLSQQGIQKGMKRGSG
TRSGLLWEIERALDSTEKNDLPKYLLMENVGALLHKKNEEELNQWKQKLESLGYQNSIEVLNAAD
FGSSQARRRVFMISTLNEFVELPKGDKKPKSIKKVLNKIVSEKDILNNLLKYNLTEFKKTKSNIN
KASLIGYSKFNSEGYVYDPEFTGPTLTASGANSRIKIKDGSNIRKMNSDETFLYIGFDSQDGKRV
NEIEFLTENQKIFVCGNSISVEVLEAIIDKIGGPSSGGKRPAATKKAGQAKKKKGSYPYDVPDYA
>Amino acid sequence of the Effector Fusion
Protein of dCas9
SEQ ID NO: 160
MAPKKKRKVGIHGVPAADKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQELD
INRLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIH
QSITGLYETRIDLSQLGGDSGGKRPAATKKAGQAKKKKGSYPYDVPDYA
>Amino acid sequence of the Effector Fusion
Protein of dCas9-KRAB
SEQ ID NO: 161
MAPKKKRKVGIHGVPAADKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQELD
INRLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIH
QSITGLYETRIDLSQLGGDKRPAATKKAGQAKKKKASDAKSLTAWSRTLVTFKDVFVDFTREEWK
LLDTAQQILYRNVMLENYKNLVSLGYQLTKPDVILRLEKGEEPWLVEREIHQETHPDSETAFEIK
SSVSGGKRPAATKKAGQAKKKKGSYPYDVPDYA
>Amino acid sequence of the Effector Fusion
Protein of G9A-dCas9
SEQ ID NO: 162
MAPKKKRKVGIHGVPAASGGGGSGNRAIRTEKIICRDVARGYENVPIPCVNGVDGEPCPEDYKYI
SENCETSTMNIDRNITHLQHCTCVDDCSSSNCLCGQLSIRCWYDKDGRLLQEFNKIEPPLIFECN
QACSCWRNCKNRVVQSGIKVRLQLYRTAKMGWGVRALQTIPQGTFICEYVGELISDAEADVREDD
SYLFDLDNKDGEVYCIDARYYGNISRFINHLCDPNIIPVRVFMLHQDLRFPRIAFFSSRDIRTGE
ELGFDYGDRFWDIKSKYFTCQCGSEKCKHSAEAIALEQSRLARLDGGGGSGDKKYSIGLAIGTNS
VGWAVITDEYKVPSKKFKVLGNTDRHSIKKNLIGALLFDSGETAEATRLKRTARRRYTRRKNRIC
YLQEIFSNEMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINASGVDAKA
ILSARLSKSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRK
LINGIRDKQSGKTILDFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVAAIVPQSFLKDDSIDNKVLTRS
DKARGKSDNVPSEEVVKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAK
GYKEVKKDLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGKRPAATKKA
GQAKKKKGSYPYDVPDYA
>Amino acid sequence of the Effector Fusion
Protein of EZH2-dCas9
SEQ ID NO: 163
MAPKKKRKVGGSGGSGQTGKKSEKGPVCWRKRVKSEYMRLRQLKRFRRADEVKSMFSSNRQKILE
RTEILNQEWKQRRIQPVHILTSVSSLRGTRECSVTSDLDFPTQVIPLKTLNAVASVPIMYSWSPL
QQNFMVEDETVLHNIPYMGDEVLDQDGTFIEELIKNYDGKVHGDRECGFINDEIFVELVNALGQY
NDDDDDDDGDDPEEREEKQKDLEDHRDDKESRPPRKFPSDKIFEAISSMFPDKGTAEELKEKYKE
LTEQQLPGALPPECTPNIDGPNAKSVQREQSLHSFHTLFCRRCFKYDCFLHPFHATPNTYKRKNT
ETALDNKPCGPQCYQHLEGAKEFAAALTAERIKTPPKRPGGRRRGRLPNNSSRPSTPTINVLESK
DTDSDREAGTETGGENNDKEEEEKKDETSSSSEANSRCQTPIKMKPNIEPPENVEWSGAEASMFR
VLIGTYYDNFCAIARLIGTKTCRQVYEFRVKESSIIAPAPAEDVDTPPRKKKRKHRLWAAHCRKI
QLKKDGSSNHVYNYQPCDHPRQPCDSSCPCVIAQNFCEKFCQCSSECQNRFPGCRCKAQCNTKQC
PCYLAVRECDPDLCLTCGAADHWDSKNVSCKNCSIQRGSKKHLLLAPSDVAGWGIFIKDPVQKNE
FISEYCGEIISQDEADRRGKVYDKYMCSFLFNLNNDFVVDATRKGNKIRFANHSVNPNCYAKVMM
VNGDHRIGIFAKRAIQTGEELFFDYRYSQADALKYVGIEREMEIPSTGGSGGSGGSGGSGGSGRP
DKKYSIGLAIGTNSVGWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYD
EHHQDLTLLKALVRQQLPEKYKEIFFDQSKNGYAGYIDGGASQEEFYKFIKPILEKMDGTEELLV
KLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVAAIVPQSF
LKDDSIDNKVLTRSDKARGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSN
IMNFFKTEITLANGEIRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSKKLKSVKELLGITIMER
SSFEKNPIDFLEAKGYKEVKKDLIIKLPKYSLFELENGRKRMLASAGELQKGNELALPSKYVNFL
YLASHYEKLKGSPEDNEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQSITGLYETRIDLSQLG
GDSGGKRPAATKKAGQAKKKKGSYPYDVPDYA
>Amino acid sequence of the Effector Fusion
Protein of dCas9-HDAC8
SEQ ID NO: 164
MAPKKKRKVGIHGVPAADKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQELD
INRLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIH
QSITGLYETRIDLSQLGGDSGGKRPAATKKAGQAKKKKSGGGGSEEPEEPADSGQSLVPVYIYSP
EYVSMCDSLAKIPKRASMVHSLIEAYALHKQMRIVKPKVASMEEMATFHTDAYLQHLQKVSQEGD
DDHPDSIEYGLGYDCPATEGIFDYAAAIGGATITAAQCLIDGMCKVAINWSGGWHHAKKDEASGF
CYLNDAVLGILRLRRKFERILYVDLDLHHGDGVEDAFSFTSKVMTVSLHKFSPGFFPGTGDVSDV
GLGKGRYYSVNVPIQDGIQDEKYYQICESVLKEVYQAFNPKAVVLQLGADTIAGDPMCSFNMTPV
GIGKCLKYILQWQLATLILGGGGYNLANTARCWTYLTGVILGKTLSSEIPDHEFFTAYGPDYVLE
ITPSCRPDRNEPHRIQQILNYIKGNLKHVVGGGGSGKRPAATKKAGQAKKKKGSYPYDVPDYA
> Nucleotide sequence of the Effector Fusion
Protein of dCas9-MQ1
SEQ ID NO: 165
AAGGAAAUAAGAGAGAAAAGAAGAGUAAGAAGAAAUAUAAGAGCCACCAUGGCCCCCAAGAAGAA
GCGGAAGGUGGGCAUCCACGGCGUGCCCGCCGCCGACAAGAAGUACAGCAUCGGCCUGGCCAUCG
GCACCAACAGCGUGGGCUGGGCCGUGAUCACCGACGAGUACAAGGUGCCCAGCAAGAAGUUCAAG
GUGCUGGGCAACACCGACCGGCACAGCAUCAAGAAGAACCUGAUCGGCGCCCUGCUGUUCGACAG
CGGCGAGACCGCCGAGGCCACCCGGCUGAAGCGGACCGCCCGGCGGCGGUACACCCGGCGGAAGA
ACCGGAUCUGCUACCUGCAGGAGAUCUUCAGCAACGAGAUGGCCAAGGUGGACGACAGCUUCUUC
CACCGGCUGGAGGAGAGCUUCCUGGUGGAGGAGGACAAGAAGCACGAGCGGCACCCCAUCUUCGG
CAACAUCGUGGACGAGGUGGCCUACCACGAGAAGUACCCCACCAUCUACCACCUGCGGAAGAAGC
UGGUGGACAGCACCGACAAGGCCGACCUGCGGCUGAUCUACCUGGCCCUGGCCCACAUGAUCAAG
UUCCGGGGCCACUUCCUGAUCGAGGGCGACCUGAACCCCGACAACAGCGACGUGGACAAGCUGUU
CAUCCAGCUGGUGCAGACCUACAACCAGCUGUUCGAGGAGAACCCCAUCAACGCCAGCGGCGUGG
ACGCCAAGGCCAUCCUGAGCGCCCGGCUGAGCAAGAGCCGGCGGCUGGAGAACCUGAUCGCCCAG
CUGCCCGGCGAGAAGAAGAACGGCCUGUUCGGCAACCUGAUCGCCCUGAGCCUGGGCCUGACCCC
CAACUUCAAGAGCAACUUCGACCUGGCCGAGGACGCCAAGCUGCAGCUGAGCAAGGACACCUACG
ACGACGACCUGGACAACCUGCUGGCCCAGAUCGGCGACCAGUACGCCGACCUGUUCCUGGCCGCC
AAGAACCUGAGCGACGCCAUCCUGCUGAGCGACAUCCUGCGGGUGAACACCGAGAUCACCAAGGC
CCCCCUGAGCGCCAGCAUGAUCAAGCGGUACGACGAGCACCACCAGGACCUGACCCUGCUGAAGG
CCCUGGUGCGGCAGCAGCUGCCCGAGAAGUACAAGGAGAUCUUCUUCGACCAGAGCAAGAACGGC
UACGCCGGCUACAUCGACGGCGGCGCCAGCCAGGAGGAGUUCUACAAGUUCAUCAAGCCCAUCCU
GGAGAAGAUGGACGGCACCGAGGAGCUGCUGGUGAAGCUGAACCGGGAGGACCUGCUGCGGAAGC
AGCGGACCUUCGACAACGGCAGCAUCCCCCACCAGAUCCACCUGGGCGAGCUGCACGCCAUCCUG
CGGCGGCAGGAGGACUUCUACCCCUUCCUGAAGGACAACCGGGAGAAGAUCGAGAAGAUCCUGAC
CUUCCGGAUCCCCUACUACGUGGGCCCCCUGGCCCGGGGCAACAGCCGGUUCGCCUGGAUGACCC
GGAAAUCCGAGGAGACCAUCACCCCCUGGAACUUCGAGGAGGUGGUGGACAAGGGCGCCAGCGCC
CAGAGCUUCAUCGAGCGGAUGACCAACUUCGACAAGAACCUGCCCAACGAGAAGGUGCUGCCCAA
GCACAGCCUGCUGUACGAGUACUUCACCGUGUACAACGAGCUGACCAAGGUGAAGUACGUGACCG
AGGGCAUGCGGAAGCCCGCCUUCCUGAGCGGCGAGCAGAAGAAGGCCAUCGUGGACCUGCUGUUC
AAGACCAACCGGAAGGUGACCGUGAAGCAGCUGAAGGAGGACUACUUCAAGAAGAUCGAGUGCUU
CGACAGCGUGGAGAUCAGCGGCGUGGAGGACCGGUUCAACGCCAGCCUGGGCACCUACCACGACC
UGCUGAAGAUCAUCAAGGACAAGGACUUCCUGGACAACGAGGAGAACGAGGACAUCCUGGAGGAC
AUCGUGCUGACCCUGACCCUGUUCGAGGACCGGGAGAUGAUCGAGGAGCGGCUGAAAACCUACGC
CCACCUGUUCGACGACAAGGUGAUGAAGCAGCUGAAGCGGCGGCGGUACACCGGCUGGGGCCGGC
UGAGCCGGAAGCUGAUCAACGGCAUCCGGGACAAGCAGAGCGGCAAGACCAUCCUGGACUUCCUG
AAAUCCGACGGCUUCGCCAACCGGAACUUCAUGCAGCUGAUCCACGACGACAGCCUGACCUUCAA
GGAGGACAUCCAGAAGGCCCAGGUGAGCGGCCAGGGCGACAGCCUGCACGAGCACAUCGCCAACC
UGGCCGGCAGCCCCGCCAUCAAGAAGGGCAUCCUGCAGACCGUGAAGGUGGUGGACGAGCUGGUG
AAGGUGAUGGGCCGGCACAAGCCCGAGAACAUCGUGAUCGAGAUGGCCCGGGAGAACCAGACCAC
CCAGAAGGGCCAGAAGAACAGCCGGGAGCGGAUGAAGCGGAUCGAGGAGGGCAUCAAGGAGCUGG
GCAGCCAGAUCCUGAAGGAGCACCCCGUGGAGAACACCCAGCUGCAGAACGAGAAGCUGUACCUG
UACUACCUGCAGAACGGCCGGGACAUGUACGUGGACCAGGAGCUGGACAUCAACCGGCUGAGCGA
CUACGACGUGGCCGCCAUCGUGCCCCAGAGCUUCCUGAAGGACGACAGCAUCGACAACAAGGUGC
UGACCCGGAGCGACAAGGCCCGGGGCAAGAGCGACAACGUGCCCAGCGAGGAGGUGGUGAAGAAG
AUGAAGAACUACUGGCGGCAGCUGCUGAACGCCAAGCUGAUCACCCAGCGGAAGUUCGACAACCU
GACCAAGGCCGAGCGGGGCGGCCUGAGCGAGCUGGACAAGGCCGGCUUCAUCAAGCGGCAGCUGG
UGGAGACCCGGCAGAUCACCAAGCACGUGGCCCAGAUCCUGGACAGCCGGAUGAACACCAAGUAC
GACGAGAACGACAAGCUGAUCCGGGAGGUGAAGGUGAUCACCCUGAAAUCCAAGCUGGUGAGCGA
CUUCCGGAAGGACUUCCAGUUCUACAAGGUGCGGGAGAUCAACAACUACCACCACGCCCACGACG
CCUACCUGAACGCCGUGGUGGGCACCGCCCUGAUCAAGAAGUACCCCAAGCUGGAGAGCGAGUUC
GUGUACGGCGACUACAAGGUGUACGACGUGCGGAAGAUGAUCGCCAAGAGCGAGCAGGAGAUCGG
CAAGGCCACCGCCAAGUACUUCUUCUACAGCAACAUCAUGAACUUCUUCAAGACCGAGAUCACCC
UGGCCAACGGCGAGAUCCGGAAGCGGCCCCUGAUCGAGACCAACGGCGAGACCGGCGAGAUCGUG
UGGGACAAGGGCCGGGACUUCGCCACCGUGCGGAAGGUGCUGAGCAUGCCCCAGGUGAACAUCGU
GAAGAAAACCGAGGUGCAGACCGGCGGCUUCAGCAAGGAGAGCAUCCUGCCCAAGCGGAACAGCG
ACAAGCUGAUCGCCCGGAAGAAGGACUGGGACCCCAAGAAGUACGGCGGCUUCGACAGCCCCACC
GUGGCCUACAGCGUGCUGGUGGUGGCCAAGGUGGAGAAGGGCAAGAGCAAGAAGCUGAAAUCCGU
GAAGGAGCUGCUGGGCAUCACCAUCAUGGAGCGGAGCAGCUUCGAGAAGAACCCCAUCGACUUCC
UGGAGGCCAAGGGCUACAAGGAGGUGAAGAAGGACCUGAUCAUCAAGCUGCCCAAGUACAGCCUG
UUCGAGCUGGAGAACGGCCGGAAGCGGAUGCUGGCCAGCGCCGGCGAGCUGCAGAAGGGCAACGA
GCUGGCCCUGCCCAGCAAGUACGUGAACUUCCUGUACCUGGCCAGCCACUACGAGAAGCUGAAGG
GCAGCCCCGAGGACAACGAGCAGAAGCAGCUGUUCGUGGAGCAGCACAAGCACUACCUGGACGAG
AUCAUCGAGCAGAUCAGCGAGUUCAGCAAGCGGGUGAUCCUGGCCGACGCCAACCUGGACAAGGU
GCUGAGCGCCUACAACAAGCACCGGGACAAGCCCAUCCGGGAGCAGGCCGAGAACAUCAUCCACC
UGUUCACCCUGACCAACCUGGGCGCCCCCGCCGCCUUCAAGUACUUCGACACCACCAUCGACCGG
AAGCGGUACACCAGCACCAAGGAGGUGCUGGACGCCACCCUGAUCCACCAGAGCAUCACCGGCCU
GUACGAGACCCGGAUCGACCUGAGCCAGCUGGGCGGCGACAAGCGGCCCGCCGCCACCAAGAAGG
CCGGCCAGGCCAAGAAGAAGAAGGCCCGGGACAGCAAGGUGGAGAACAAGACCAAGAAGCUGCGG
GUGUUCGAGGCCUUCGCCGGCAUCGGCGCCCAGCGGAAGGCCCUGGAGAAGGUGCGGAAGGACGA
GUACGAGAUCGUGGGCCUGGCCGAGUGGUACGUGCCCGCCAUCGUGAUGUACCAGGCCAUCCACA
ACAACUUCCACACCAAGCUGGAGUACAAGAGCGUGAGCCGGGAGGAGAUGAUCGACUACCUGGAG
AACAAGACCCUGAGCUGGAACAGCAAGAACCCCGUGAGCAACGGCUACUGGAAGCGGAAGAAGGA
CGACGAGCUGAAGAUCAUCUACAACGCCAUCAAGCUGAGCGAGAAGGAGGGCAACAUCUUCGACA
UCCGGGACCUGUACAAGCGGACCCUGAAGAACAUCGACCUGCUGACCUACAGCUUCCCCUGCCAG
GACCUGAGCCAGCAGGGCAUCCAGAAGGGCAUGAAGCGGGGCAGCGGCACCCGGAGCGGCCUGCU
GUGGGAGAUCGAGCGGGCCCUGGACAGCACCGAGAAGAACGACCUGCCCAAGUACCUGCUGAUGG
AGAACGUGGGCGCCCUGCUGCACAAGAAGAACGAGGAGGAGCUGAACCAGUGGAAGCAGAAGCUG
GAGAGCCUGGGCUACCAGAACAGCAUCGAGGUGCUGAACGCCGCCGACUUCGGCAGCAGCCAGGC
CCGGCGGCGGGUGUUCAUGAUCAGCACCCUGAACGAGUUCGUGGAGCUGCCCAAGGGCGACAAGA
AGCCCAAGAGCAUCAAGAAGGUGCUGAACAAGAUCGUGAGCGAGAAGGACAUCCUGAACAACCUG
CUGAAGUACAACCUGACCGAGUUCAAGAAAACCAAGAGCAACAUCAACAAGGCCAGCCUGAUCGG
CUACAGCAAGUUCAACAGCGAGGGCUACGUGUACGACCCCGAGUUCACCGGCCCCACCCUGACCG
CCAGCGGCGCCAACAGCCGGAUCAAGAUCAAGGACGGCAGCAACAUCCGGAAGAUGAACAGCGAC
GAGACCUUCCUGUACAUCGGCUUCGACAGCCAGGACGGCAAGCGGGUGAACGAGAUCGAGUUCCU
GACCGAGAACCAGAAGAUCUUCGUGUGCGGCAACAGCAUCAGCGUGGAGGUGCUGGAGGCCAUCA
UCGACAAGAUCGGCGGCCCCAGCAGCGGCGGCAAGCGGCCCGCCGCCACCAAGAAGGCCGGCCAG
GCCAAGAAGAAGAAGGGCAGCUACCCCUACGACGUGCCCGACUACGCCUGAGCGGCCGCUUAAUU
AAGCUGCCUUCUGCGGGGCUUGCCUUCUGGCCAUGCCCUUCUUCUCUCCCUUGCACCUGUACCUC
UUGGUCUUUGAAUAAAGCCUGAGUAGGAAGUCUAGAAAAAAAAAAAAAAAAAAAAAAAAAAAAAA
AAAAAAAAAAAAAAAAAAAAAAAAAAAAAAAAAAAAAAAAAAAAAAAAAA
>Nucleotide sequence of the Effector Fusion
Protein of dCas9
SEQ ID NO: 166
AAGGAAAUAAGAGAGAAAAGAAGAGUAAGAAGAAAUAUAAGAGCCACCAUGGCCCCCAAGAAGAA
GCGGAAGGUGGGCAUCCACGGCGUGCCCGCCGCCGACAAGAAGUACAGCAUCGGCCUGGCCAUCG
GCACCAACAGCGUGGGCUGGGCCGUGAUCACCGACGAGUACAAGGUGCCCAGCAAGAAGUUCAAG
GUGCUGGGCAACACCGACCGGCACAGCAUCAAGAAGAACCUGAUCGGCGCCCUGCUGUUCGACAG
CGGCGAGACCGCCGAGGCCACCCGGCUGAAGCGGACCGCCCGGCGGCGGUACACCCGGCGGAAGA
ACCGGAUCUGCUACCUGCAGGAGAUCUUCAGCAACGAGAUGGCCAAGGUGGACGACAGCUUCUUC
CACCGGCUGGAGGAGAGCUUCCUGGUGGAGGAGGACAAGAAGCACGAGCGGCACCCCAUCUUCGG
CAACAUCGUGGACGAGGUGGCCUACCACGAGAAGUACCCCACCAUCUACCACCUGCGGAAGAAGC
UGGUGGACAGCACCGACAAGGCCGACCUGCGGCUGAUCUACCUGGCCCUGGCCCACAUGAUCAAG
UUCCGGGGCCACUUCCUGAUCGAGGGCGACCUGAACCCCGACAACAGCGACGUGGACAAGCUGUU
CAUCCAGCUGGUGCAGACCUACAACCAGCUGUUCGAGGAGAACCCCAUCAACGCCAGCGGCGUGG
ACGCCAAGGCCAUCCUGAGCGCCCGGCUGAGCAAGAGCCGGCGGCUGGAGAACCUGAUCGCCCAG
CUGCCCGGCGAGAAGAAGAACGGCCUGUUCGGCAACCUGAUCGCCCUGAGCCUGGGCCUGACCCC
CAACUUCAAGAGCAACUUCGACCUGGCCGAGGACGCCAAGCUGCAGCUGAGCAAGGACACCUACG
ACGACGACCUGGACAACCUGCUGGCCCAGAUCGGCGACCAGUACGCCGACCUGUUCCUGGCCGCC
AAGAACCUGAGCGACGCCAUCCUGCUGAGCGACAUCCUGCGGGUGAACACCGAGAUCACCAAGGC
CCCCCUGAGCGCCAGCAUGAUCAAGCGGUACGACGAGCACCACCAGGACCUGACCCUGCUGAAGG
CCCUGGUGCGGCAGCAGCUGCCCGAGAAGUACAAGGAGAUCUUCUUCGACCAGAGCAAGAACGGC
UACGCCGGCUACAUCGACGGCGGCGCCAGCCAGGAGGAGUUCUACAAGUUCAUCAAGCCCAUCCU
GGAGAAGAUGGACGGCACCGAGGAGCUGCUGGUGAAGCUGAACCGGGAGGACCUGCUGCGGAAGC
AGCGGACCUUCGACAACGGCAGCAUCCCCCACCAGAUCCACCUGGGCGAGCUGCACGCCAUCCUG
CGGCGGCAGGAGGACUUCUACCCCUUCCUGAAGGACAACCGGGAGAAGAUCGAGAAGAUCCUGAC
CUUCCGGAUCCCCUACUACGUGGGCCCCCUGGCCCGGGGCAACAGCCGGUUCGCCUGGAUGACCC
GGAAAUCCGAGGAGACCAUCACCCCCUGGAACUUCGAGGAGGUGGUGGACAAGGGCGCCAGCGCC
CAGAGCUUCAUCGAGCGGAUGACCAACUUCGACAAGAACCUGCCCAACGAGAAGGUGCUGCCCAA
GCACAGCCUGCUGUACGAGUACUUCACCGUGUACAACGAGCUGACCAAGGUGAAGUACGUGACCG
AGGGCAUGCGGAAGCCCGCCUUCCUGAGCGGCGAGCAGAAGAAGGCCAUCGUGGACCUGCUGUUC
AAGACCAACCGGAAGGUGACCGUGAAGCAGCUGAAGGAGGACUACUUCAAGAAGAUCGAGUGCUU
CGACAGCGUGGAGAUCAGCGGCGUGGAGGACCGGUUCAACGCCAGCCUGGGCACCUACCACGACC
UGCUGAAGAUCAUCAAGGACAAGGACUUCCUGGACAACGAGGAGAACGAGGACAUCCUGGAGGAC
AUCGUGCUGACCCUGACCCUGUUCGAGGACCGGGAGAUGAUCGAGGAGCGGCUGAAAACCUACGC
CCACCUGUUCGACGACAAGGUGAUGAAGCAGCUGAAGCGGCGGCGGUACACCGGCUGGGGCCGGC
UGAGCCGGAAGCUGAUCAACGGCAUCCGGGACAAGCAGAGCGGCAAGACCAUCCUGGACUUCCUG
AAAUCCGACGGCUUCGCCAACCGGAACUUCAUGCAGCUGAUCCACGACGACAGCCUGACCUUCAA
GGAGGACAUCCAGAAGGCCCAGGUGAGCGGCCAGGGCGACAGCCUGCACGAGCACAUCGCCAACC
UGGCCGGCAGCCCCGCCAUCAAGAAGGGCAUCCUGCAGACCGUGAAGGUGGUGGACGAGCUGGUG
AAGGUGAUGGGCCGGCACAAGCCCGAGAACAUCGUGAUCGAGAUGGCCCGGGAGAACCAGACCAC
CCAGAAGGGCCAGAAGAACAGCCGGGAGCGGAUGAAGCGGAUCGAGGAGGGCAUCAAGGAGCUGG
GCAGCCAGAUCCUGAAGGAGCACCCCGUGGAGAACACCCAGCUGCAGAACGAGAAGCUGUACCUG
UACUACCUGCAGAACGGCCGGGACAUGUACGUGGACCAGGAGCUGGACAUCAACCGGCUGAGCGA
CUACGACGUGGCCGCCAUCGUGCCCCAGAGCUUCCUGAAGGACGACAGCAUCGACAACAAGGUGC
UGACCCGGAGCGACAAGGCCCGGGGCAAGAGCGACAACGUGCCCAGCGAGGAGGUGGUGAAGAAG
AUGAAGAACUACUGGCGGCAGCUGCUGAACGCCAAGCUGAUCACCCAGCGGAAGUUCGACAACCU
GACCAAGGCCGAGCGGGGCGGCCUGAGCGAGCUGGACAAGGCCGGCUUCAUCAAGCGGCAGCUGG
UGGAGACCCGGCAGAUCACCAAGCACGUGGCCCAGAUCCUGGACAGCCGGAUGAACACCAAGUAC
GACGAGAACGACAAGCUGAUCCGGGAGGUGAAGGUGAUCACCCUGAAAUCCAAGCUGGUGAGCGA
CUUCCGGAAGGACUUCCAGUUCUACAAGGUGCGGGAGAUCAACAACUACCACCACGCCCACGACG
CCUACCUGAACGCCGUGGUGGGCACCGCCCUGAUCAAGAAGUACCCCAAGCUGGAGAGCGAGUUC
GUGUACGGCGACUACAAGGUGUACGACGUGCGGAAGAUGAUCGCCAAGAGCGAGCAGGAGAUCGG
CAAGGCCACCGCCAAGUACUUCUUCUACAGCAACAUCAUGAACUUCUUCAAGACCGAGAUCACCC
UGGCCAACGGCGAGAUCCGGAAGCGGCCCCUGAUCGAGACCAACGGCGAGACCGGCGAGAUCGUG
UGGGACAAGGGCCGGGACUUCGCCACCGUGCGGAAGGUGCUGAGCAUGCCCCAGGUGAACAUCGU
GAAGAAAACCGAGGUGCAGACCGGCGGCUUCAGCAAGGAGAGCAUCCUGCCCAAGCGGAACAGCG
ACAAGCUGAUCGCCCGGAAGAAGGACUGGGACCCCAAGAAGUACGGCGGCUUCGACAGCCCCACC
GUGGCCUACAGCGUGCUGGUGGUGGCCAAGGUGGAGAAGGGCAAGAGCAAGAAGCUGAAAUCCGU
GAAGGAGCUGCUGGGCAUCACCAUCAUGGAGCGGAGCAGCUUCGAGAAGAACCCCAUCGACUUCC
UGGAGGCCAAGGGCUACAAGGAGGUGAAGAAGGACCUGAUCAUCAAGCUGCCCAAGUACAGCCUG
UUCGAGCUGGAGAACGGCCGGAAGCGGAUGCUGGCCAGCGCCGGCGAGCUGCAGAAGGGCAACGA
GCUGGCCCUGCCCAGCAAGUACGUGAACUUCCUGUACCUGGCCAGCCACUACGAGAAGCUGAAGG
GCAGCCCCGAGGACAACGAGCAGAAGCAGCUGUUCGUGGAGCAGCACAAGCACUACCUGGACGAG
AUCAUCGAGCAGAUCAGCGAGUUCAGCAAGCGGGUGAUCCUGGCCGACGCCAACCUGGACAAGGU
GCUGAGCGCCUACAACAAGCACCGGGACAAGCCCAUCCGGGAGCAGGCCGAGAACAUCAUCCACC
UGUUCACCCUGACCAACCUGGGCGCCCCCGCCGCCUUCAAGUACUUCGACACCACCAUCGACCGG
AAGCGGUACACCAGCACCAAGGAGGUGCUGGACGCCACCCUGAUCCACCAGAGCAUCACCGGCCU
GUACGAGACCCGGAUCGACCUGAGCCAGCUGGGCGGCGACAGCGGCGGCAAGCGGCCCGCCGCCA
CCAAGAAGGCCGGCCAGGCCAAGAAGAAGAAGGGCAGCUACCCCUACGACGUGCCCGACUACGCC
UGAGCGGCCGCUUAAUUAAGCUGCCUUCUGCGGGGCUUGCCUUCUGGCCAUGCCCUUCUUCUCUC
CCUUGCACCUGUACCUCUUGGUCUUUGAAUAAAGCCUGAGUAGGAAGUCUAGAAAAAAAAAAAAA
AAAAAAAAAAAAAAAAAAAAAAAAAAAAAAAAAAAAAAAAAAAAAAAAAAAAAAAAAAAAAAAAA
AA
>Nucleotide sequence of the Effector Fusion
Protein of dCas9-KRAB
SEQ ID NO: 167
AAGGAAAUAAGAGAGAAAAGAAGAGUAAGAAGAAAUAUAAGAGCCACCAUGGCCCCCAAGAAGAA
GCGGAAGGUGGGCAUCCACGGCGUGCCCGCCGCCGACAAGAAGUACAGCAUCGGCCUGGCCAUCG
GCACCAACAGCGUGGGCUGGGCCGUGAUCACCGACGAGUACAAGGUGCCCAGCAAGAAGUUCAAG
GUGCUGGGCAACACCGACCGGCACAGCAUCAAGAAGAACCUGAUCGGCGCCCUGCUGUUCGACAG
CGGCGAGACCGCCGAGGCCACCCGGCUGAAGCGGACCGCCCGGCGGCGGUACACCCGGCGGAAGA
ACCGGAUCUGCUACCUGCAGGAGAUCUUCAGCAACGAGAUGGCCAAGGUGGACGACAGCUUCUUC
CACCGGCUGGAGGAGAGCUUCCUGGUGGAGGAGGACAAGAAGCACGAGCGGCACCCCAUCUUCGG
CAACAUCGUGGACGAGGUGGCCUACCACGAGAAGUACCCCACCAUCUACCACCUGCGGAAGAAGC
UGGUGGACAGCACCGACAAGGCCGACCUGCGGCUGAUCUACCUGGCCCUGGCCCACAUGAUCAAG
UUCCGGGGCCACUUCCUGAUCGAGGGCGACCUGAACCCCGACAACAGCGACGUGGACAAGCUGUU
CAUCCAGCUGGUGCAGACCUACAACCAGCUGUUCGAGGAGAACCCCAUCAACGCCAGCGGCGUGG
ACGCCAAGGCCAUCCUGAGCGCCCGGCUGAGCAAGAGCCGGCGGCUGGAGAACCUGAUCGCCCAG
CUGCCCGGCGAGAAGAAGAACGGCCUGUUCGGCAACCUGAUCGCCCUGAGCCUGGGCCUGACCCC
CAACUUCAAGAGCAACUUCGACCUGGCCGAGGACGCCAAGCUGCAGCUGAGCAAGGACACCUACG
ACGACGACCUGGACAACCUGCUGGCCCAGAUCGGCGACCAGUACGCCGACCUGUUCCUGGCCGCC
AAGAACCUGAGCGACGCCAUCCUGCUGAGCGACAUCCUGCGGGUGAACACCGAGAUCACCAAGGC
CCCCCUGAGCGCCAGCAUGAUCAAGCGGUACGACGAGCACCACCAGGACCUGACCCUGCUGAAGG
CCCUGGUGCGGCAGCAGCUGCCCGAGAAGUACAAGGAGAUCUUCUUCGACCAGAGCAAGAACGGC
UACGCCGGCUACAUCGACGGCGGCGCCAGCCAGGAGGAGUUCUACAAGUUCAUCAAGCCCAUCCU
GGAGAAGAUGGACGGCACCGAGGAGCUGCUGGUGAAGCUGAACCGGGAGGACCUGCUGCGGAAGC
AGCGGACCUUCGACAACGGCAGCAUCCCCCACCAGAUCCACCUGGGCGAGCUGCACGCCAUCCUG
CGGCGGCAGGAGGACUUCUACCCCUUCCUGAAGGACAACCGGGAGAAGAUCGAGAAGAUCCUGAC
CUUCCGGAUCCCCUACUACGUGGGCCCCCUGGCCCGGGGCAACAGCCGGUUCGCCUGGAUGACCC
GGAAAUCCGAGGAGACCAUCACCCCCUGGAACUUCGAGGAGGUGGUGGACAAGGGCGCCAGCGCC
CAGAGCUUCAUCGAGCGGAUGACCAACUUCGACAAGAACCUGCCCAACGAGAAGGUGCUGCCCAA
GCACAGCCUGCUGUACGAGUACUUCACCGUGUACAACGAGCUGACCAAGGUGAAGUACGUGACCG
AGGGCAUGCGGAAGCCCGCCUUCCUGAGCGGCGAGCAGAAGAAGGCCAUCGUGGACCUGCUGUUC
AAGACCAACCGGAAGGUGACCGUGAAGCAGCUGAAGGAGGACUACUUCAAGAAGAUCGAGUGCUU
CGACAGCGUGGAGAUCAGCGGCGUGGAGGACCGGUUCAACGCCAGCCUGGGCACCUACCACGACC
UGCUGAAGAUCAUCAAGGACAAGGACUUCCUGGACAACGAGGAGAACGAGGACAUCCUGGAGGAC
AUCGUGCUGACCCUGACCCUGUUCGAGGACCGGGAGAUGAUCGAGGAGCGGCUGAAAACCUACGC
CCACCUGUUCGACGACAAGGUGAUGAAGCAGCUGAAGCGGCGGCGGUACACCGGCUGGGGCCGGC
UGAGCCGGAAGCUGAUCAACGGCAUCCGGGACAAGCAGAGCGGCAAGACCAUCCUGGACUUCCUG
AAAUCCGACGGCUUCGCCAACCGGAACUUCAUGCAGCUGAUCCACGACGACAGCCUGACCUUCAA
GGAGGACAUCCAGAAGGCCCAGGUGAGCGGCCAGGGCGACAGCCUGCACGAGCACAUCGCCAACC
UGGCCGGCAGCCCCGCCAUCAAGAAGGGCAUCCUGCAGACCGUGAAGGUGGUGGACGAGCUGGUG
AAGGUGAUGGGCCGGCACAAGCCCGAGAACAUCGUGAUCGAGAUGGCCCGGGAGAACCAGACCAC
CCAGAAGGGCCAGAAGAACAGCCGGGAGCGGAUGAAGCGGAUCGAGGAGGGCAUCAAGGAGCUGG
GCAGCCAGAUCCUGAAGGAGCACCCCGUGGAGAACACCCAGCUGCAGAACGAGAAGCUGUACCUG
UACUACCUGCAGAACGGCCGGGACAUGUACGUGGACCAGGAGCUGGACAUCAACCGGCUGAGCGA
CUACGACGUGGCCGCCAUCGUGCCCCAGAGCUUCCUGAAGGACGACAGCAUCGACAACAAGGUGC
UGACCCGGAGCGACAAGGCCCGGGGCAAGAGCGACAACGUGCCCAGCGAGGAGGUGGUGAAGAAG
AUGAAGAACUACUGGCGGCAGCUGCUGAACGCCAAGCUGAUCACCCAGCGGAAGUUCGACAACCU
GACCAAGGCCGAGCGGGGCGGCCUGAGCGAGCUGGACAAGGCCGGCUUCAUCAAGCGGCAGCUGG
UGGAGACCCGGCAGAUCACCAAGCACGUGGCCCAGAUCCUGGACAGCCGGAUGAACACCAAGUAC
GACGAGAACGACAAGCUGAUCCGGGAGGUGAAGGUGAUCACCCUGAAAUCCAAGCUGGUGAGCGA
CUUCCGGAAGGACUUCCAGUUCUACAAGGUGCGGGAGAUCAACAACUACCACCACGCCCACGACG
CCUACCUGAACGCCGUGGUGGGCACCGCCCUGAUCAAGAAGUACCCCAAGCUGGAGAGCGAGUUC
GUGUACGGCGACUACAAGGUGUACGACGUGCGGAAGAUGAUCGCCAAGAGCGAGCAGGAGAUCGG
CAAGGCCACCGCCAAGUACUUCUUCUACAGCAACAUCAUGAACUUCUUCAAGACCGAGAUCACCC
UGGCCAACGGCGAGAUCCGGAAGCGGCCCCUGAUCGAGACCAACGGCGAGACCGGCGAGAUCGUG
UGGGACAAGGGCCGGGACUUCGCCACCGUGCGGAAGGUGCUGAGCAUGCCCCAGGUGAACAUCGU
GAAGAAAACCGAGGUGCAGACCGGCGGCUUCAGCAAGGAGAGCAUCCUGCCCAAGCGGAACAGCG
ACAAGCUGAUCGCCCGGAAGAAGGACUGGGACCCCAAGAAGUACGGCGGCUUCGACAGCCCCACC
GUGGCCUACAGCGUGCUGGUGGUGGCCAAGGUGGAGAAGGGCAAGAGCAAGAAGCUGAAAUCCGU
GAAGGAGCUGCUGGGCAUCACCAUCAUGGAGCGGAGCAGCUUCGAGAAGAACCCCAUCGACUUCC
UGGAGGCCAAGGGCUACAAGGAGGUGAAGAAGGACCUGAUCAUCAAGCUGCCCAAGUACAGCCUG
UUCGAGCUGGAGAACGGCCGGAAGCGGAUGCUGGCCAGCGCCGGCGAGCUGCAGAAGGGCAACGA
GCUGGCCCUGCCCAGCAAGUACGUGAACUUCCUGUACCUGGCCAGCCACUACGAGAAGCUGAAGG
GCAGCCCCGAGGACAACGAGCAGAAGCAGCUGUUCGUGGAGCAGCACAAGCACUACCUGGACGAG
AUCAUCGAGCAGAUCAGCGAGUUCAGCAAGCGGGUGAUCCUGGCCGACGCCAACCUGGACAAGGU
GCUGAGCGCCUACAACAAGCACCGGGACAAGCCCAUCCGGGAGCAGGCCGAGAACAUCAUCCACC
UGUUCACCCUGACCAACCUGGGCGCCCCCGCCGCCUUCAAGUACUUCGACACCACCAUCGACCGG
AAGCGGUACACCAGCACCAAGGAGGUGCUGGACGCCACCCUGAUCCACCAGAGCAUCACCGGCCU
GUACGAGACCCGGAUCGACCUGAGCCAGCUGGGCGGCGACAAGCGGCCCGCCGCCACCAAGAAGG
CCGGCCAGGCCAAGAAGAAGAAGGCCAGCGACGCCAAGAGCCUGACCGCCUGGAGCCGGACCCUG
GUGACCUUCAAGGACGUGUUCGUGGACUUCACCCGGGAGGAGUGGAAGCUGCUGGACACCGCCCA
GCAGAUCCUGUACCGGAACGUGAUGCUGGAGAACUACAAGAACCUGGUGAGCCUGGGCUACCAGC
UGACCAAGCCCGACGUGAUCCUGCGGCUGGAGAAGGGCGAGGAGCCCUGGCUGGUGGAGCGGGAG
AUCCACCAGGAGACCCACCCCGACAGCGAGACCGCCUUCGAGAUCAAGAGCAGCGUGAGCGGCGG
CAAGCGGCCCGCCGCCACCAAGAAGGCCGGCCAGGCCAAGAAGAAGAAGGGCAGCUACCCCUACG
ACGUGCCCGACUACGCCUGAGCGGCCGCUUAAUUAAGCUGCCUUCUGCGGGGCUUGCCUUCUGGC
CAUGCCCUUCUUCUCUCCCUUGCACCUGUACCUCUUGGUCUUUGAAUAAAGCCUGAGUAGGAAGU
CUAGAAAAAAAAAAAAAAAAAAAAAAAAAAAAAAAAAAAAAAAAAAAAAAAAAAAAAAAAAAAAA
AAAAAAAAAAAAAAAAAAA
> Nucleotide sequence of the Effector Fusion
Protein of G9A-dCas9
SEQ ID NO: 168
AGGAAAUAAGAGAGAAAAGAAGAGUAAGAAGAAAUAUAAGAGCCACCAUGGCCCCCAAGAAGAAG
CGGAAGGUGGGCAUCCACGGCGUGCCCGCCGCCUCGGGCGGGGGUGGCUCAGGAAAUAGGGCUAU
CAGAACCGAGAAGAUCAUCUGUAGGGACGUGGCUAGAGGCUACGAGAACGUGCCCAUUCCUUGCG
UGAAUGGCGUGGAUGGCGAACCUUGCCCCGAGGACUACAAAUACAUCUCCGAGAACUGCGAAACC
AGCACAAUGAACAUCGACAGAAACAUCACCCACCUCCAGCACUGCACAUGUGUGGAUGACUGCUC
CUCCAGCAACUGUCUGUGCGGCCAGCUCUCCAUCAGAUGCUGGUACGACAAGGACGGCAGACUGC
UGCAAGAGUUCAACAAGAUCGAACCCCCUCUCAUCUUCGAGUGUAACCAAGCUUGCAGCUGCUGG
AGAAACUGCAAGAAUAGAGUGGUCCAGAGCGGCAUCAAGGUGAGACUGCAACUGUACAGAACCGC
CAAGAUGGGAUGGGGAGUGAGGGCUCUGCAAACCAUUCCCCAAGGCACCUUCAUCUGCGAAUACG
UGGGCGAACUGAUCUCCGACGCCGAAGCUGACGUGAGAGAGGACGACAGCUAUCUCUUCGAUCUG
GACAAUAAGGACGGCGAGGUGUACUGCAUCGACGCUAGAUAUUACGGCAACAUCUCUAGAUUCAU
CAACCACCUCUGCGAUCCCAACAUCAUUCCCGUGAGGGUGUUCAUGCUGCACCAAGAUCUGAGGU
UCCCUAGAAUCGCCUUCUUCAGCUCUAGAGACAUCAGAACCGGCGAGGAGCUGGGCUUCGAUUAC
GGCGAUAGAUUCUGGGACAUCAAGUCCAAGUACUUCACAUGCCAGUGCGGCAGCGAGAAGUGUAA
GCACAGCGCUGAGGCCAUUGCUCUGGAGCAGUCUAGACUGGCCAGACUGGAUGGAGGUGGCGGAU
CGGGAGACAAGAAGUACAGCAUCGGCCUGGCCAUCGGCACCAACAGCGUGGGCUGGGCCGUGAUC
ACCGACGAGUACAAGGUGCCCAGCAAGAAGUUCAAGGUGCUGGGCAACACCGACCGGCACAGCAU
CAAGAAGAACCUGAUCGGCGCCCUGCUGUUCGACAGCGGCGAGACCGCCGAGGCCACCCGGCUGA
AGCGGACCGCCCGGCGGCGGUACACCCGGCGGAAGAACCGGAUCUGCUACCUGCAGGAGAUCUUC
AGCAACGAGAUGGCCAAGGUGGACGACAGCUUCUUCCACCGGCUGGAGGAGAGCUUCCUGGUGGA
GGAGGACAAGAAGCACGAGCGGCACCCCAUCUUCGGCAACAUCGUGGACGAGGUGGCCUACCACG
AGAAGUACCCCACCAUCUACCACCUGCGGAAGAAGCUGGUGGACAGCACCGACAAGGCCGACCUG
CGGCUGAUCUACCUGGCCCUGGCCCACAUGAUCAAGUUCCGGGGCCACUUCCUGAUCGAGGGCGA
CCUGAACCCCGACAACAGCGACGUGGACAAGCUGUUCAUCCAGCUGGUGCAGACCUACAACCAGC
UGUUCGAGGAGAACCCCAUCAACGCCAGCGGCGUGGACGCCAAGGCCAUCCUGAGCGCCCGGCUG
AGCAAGAGCCGGCGGCUGGAGAACCUGAUCGCCCAGCUGCCCGGCGAGAAGAAGAACGGCCUGUU
CGGCAACCUGAUCGCCCUGAGCCUGGGCCUGACCCCCAACUUCAAGAGCAACUUCGACCUGGCCG
AGGACGCCAAGCUGCAGCUGAGCAAGGACACCUACGACGACGACCUGGACAACCUGCUGGCCCAG
AUCGGCGACCAGUACGCCGACCUGUUCCUGGCCGCCAAGAACCUGAGCGACGCCAUCCUGCUGAG
CGACAUCCUGCGGGUGAACACCGAGAUCACCAAGGCCCCCCUGAGCGCCAGCAUGAUCAAGCGGU
ACGACGAGCACCACCAGGACCUGACCCUGCUGAAGGCCCUGGUGCGGCAGCAGCUGCCCGAGAAG
UACAAGGAGAUCUUCUUCGACCAGAGCAAGAACGGCUACGCCGGCUACAUCGACGGCGGCGCCAG
CCAGGAGGAGUUCUACAAGUUCAUCAAGCCCAUCCUGGAGAAGAUGGACGGCACCGAGGAGCUGC
UGGUGAAGCUGAACCGGGAGGACCUGCUGCGGAAGCAGCGGACCUUCGACAACGGCAGCAUCCCC
CACCAGAUCCACCUGGGCGAGCUGCACGCCAUCCUGCGGCGGCAGGAGGACUUCUACCCCUUCCU
GAAGGACAACCGGGAGAAGAUCGAGAAGAUCCUGACCUUCCGGAUCCCCUACUACGUGGGCCCCC
UGGCCCGGGGCAACAGCCGGUUCGCCUGGAUGACCCGGAAAUCCGAGGAGACCAUCACCCCCUGG
AACUUCGAGGAGGUGGUGGACAAGGGCGCCAGCGCCCAGAGCUUCAUCGAGCGGAUGACCAACUU
CGACAAGAACCUGCCCAACGAGAAGGUGCUGCCCAAGCACAGCCUGCUGUACGAGUACUUCACCG
UGUACAACGAGCUGACCAAGGUGAAGUACGUGACCGAGGGCAUGCGGAAGCCCGCCUUCCUGAGC
GGCGAGCAGAAGAAGGCCAUCGUGGACCUGCUGUUCAAGACCAACCGGAAGGUGACCGUGAAGCA
GCUGAAGGAGGACUACUUCAAGAAGAUCGAGUGCUUCGACAGCGUGGAGAUCAGCGGCGUGGAGG
ACCGGUUCAACGCCAGCCUGGGCACCUACCACGACCUGCUGAAGAUCAUCAAGGACAAGGACUUC
CUGGACAACGAGGAGAACGAGGACAUCCUGGAGGACAUCGUGCUGACCCUGACCCUGUUCGAGGA
CCGGGAGAUGAUCGAGGAGCGGCUGAAAACCUACGCCCACCUGUUCGACGACAAGGUGAUGAAGC
AGCUGAAGCGGCGGCGGUACACCGGCUGGGGCCGGCUGAGCCGGAAGCUGAUCAACGGCAUCCGG
GACAAGCAGAGCGGCAAGACCAUCCUGGACUUCCUGAAAUCCGACGGCUUCGCCAACCGGAACUU
CAUGCAGCUGAUCCACGACGACAGCCUGACCUUCAAGGAGGACAUCCAGAAGGCCCAGGUGAGCG
GCCAGGGCGACAGCCUGCACGAGCACAUCGCCAACCUGGCCGGCAGCCCCGCCAUCAAGAAGGGC
AUCCUGCAGACCGUGAAGGUGGUGGACGAGCUGGUGAAGGUGAUGGGCCGGCACAAGCCCGAGAA
CAUCGUGAUCGAGAUGGCCCGGGAGAACCAGACCACCCAGAAGGGCCAGAAGAACAGCCGGGAGC
GGAUGAAGCGGAUCGAGGAGGGCAUCAAGGAGCUGGGCAGCCAGAUCCUGAAGGAGCACCCCGUG
GAGAACACCCAGCUGCAGAACGAGAAGCUGUACCUGUACUACCUGCAGAACGGCCGGGACAUGUA
CGUGGACCAGGAGCUGGACAUCAACCGGCUGAGCGACUACGACGUGGCCGCCAUCGUGCCCCAGA
GCUUCCUGAAGGACGACAGCAUCGACAACAAGGUGCUGACCCGGAGCGACAAGGCCCGGGGCAAG
AGCGACAACGUGCCCAGCGAGGAGGUGGUGAAGAAGAUGAAGAACUACUGGCGGCAGCUGCUGAA
CGCCAAGCUGAUCACCCAGCGGAAGUUCGACAACCUGACCAAGGCCGAGCGGGGCGGCCUGAGCG
AGCUGGACAAGGCCGGCUUCAUCAAGCGGCAGCUGGUGGAGACCCGGCAGAUCACCAAGCACGUG
GCCCAGAUCCUGGACAGCCGGAUGAACACCAAGUACGACGAGAACGACAAGCUGAUCCGGGAGGU
GAAGGUGAUCACCCUGAAAUCCAAGCUGGUGAGCGACUUCCGGAAGGACUUCCAGUUCUACAAGG
UGCGGGAGAUCAACAACUACCACCACGCCCACGACGCCUACCUGAACGCCGUGGUGGGCACCGCC
CUGAUCAAGAAGUACCCCAAGCUGGAGAGCGAGUUCGUGUACGGCGACUACAAGGUGUACGACGU
GCGGAAGAUGAUCGCCAAGAGCGAGCAGGAGAUCGGCAAGGCCACCGCCAAGUACUUCUUCUACA
GCAACAUCAUGAACUUCUUCAAGACCGAGAUCACCCUGGCCAACGGCGAGAUCCGGAAGCGGCCC
CUGAUCGAGACCAACGGCGAGACCGGCGAGAUCGUGUGGGACAAGGGCCGGGACUUCGCCACCGU
GCGGAAGGUGCUGAGCAUGCCCCAGGUGAACAUCGUGAAGAAAACCGAGGUGCAGACCGGCGGCU
UCAGCAAGGAGAGCAUCCUGCCCAAGCGGAACAGCGACAAGCUGAUCGCCCGGAAGAAGGACUGG
GACCCCAAGAAGUACGGCGGCUUCGACAGCCCCACCGUGGCCUACAGCGUGCUGGUGGUGGCCAA
GGUGGAGAAGGGCAAGAGCAAGAAGCUGAAAUCCGUGAAGGAGCUGCUGGGCAUCACCAUCAUGG
AGCGGAGCAGCUUCGAGAAGAACCCCAUCGACUUCCUGGAGGCCAAGGGCUACAAGGAGGUGAAG
AAGGACCUGAUCAUCAAGCUGCCCAAGUACAGCCUGUUCGAGCUGGAGAACGGCCGGAAGCGGAU
GCUGGCCAGCGCCGGCGAGCUGCAGAAGGGCAACGAGCUGGCCCUGCCCAGCAAGUACGUGAACU
UCCUGUACCUGGCCAGCCACUACGAGAAGCUGAAGGGCAGCCCCGAGGACAACGAGCAGAAGCAG
CUGUUCGUGGAGCAGCACAAGCACUACCUGGACGAGAUCAUCGAGCAGAUCAGCGAGUUCAGCAA
GCGGGUGAUCCUGGCCGACGCCAACCUGGACAAGGUGCUGAGCGCCUACAACAAGCACCGGGACA
AGCCCAUCCGGGAGCAGGCCGAGAACAUCAUCCACCUGUUCACCCUGACCAACCUGGGCGCCCCC
GCCGCCUUCAAGUACUUCGACACCACCAUCGACCGGAAGCGGUACACCAGCACCAAGGAGGUGCU
GGACGCCACCCUGAUCCACCAGAGCAUCACCGGCCUGUACGAGACCCGGAUCGACCUGAGCCAGC
UGGGCGGCGACAGCGGCGGCAAGCGGCCCGCCGCCACCAAGAAGGCCGGCCAGGCCAAGAAGAAG
AAGGGCAGCUACCCCUACGACGUGCCCGACUACGCCUGAGCGGCCGCUUAAUUAAGCUGCCUUCU
GCGGGGCUUGCCUUCUGGCCAUGCCCUUCUUCUCUCCCUUGCACCUGUACCUCUUGGUCUUUGAA
UAAAGCCUGAGUAGGAAGUCUAGAAAAAAAAAAAAAAAAAAAAAAAAAAAAAAAAAAAAAAAAAA
AAAAAAAAAAAAAAAAAAAAAAAAAAAAAAAAAAAAAA
>Nucleotide sequence of the Effector Fusion
Protein of EZH2-dCas9
SEQ ID NO: 169
AGGAAAUAAGAGAGAAAAGAAGAGUAAGAAGAAAUAUAAGAGCCACCAUGGCCCCCAAGAAGAAG
CGGAAGGUGGGCGGCAGCGGCGGCAGCGGCCAGACCGGCAAGAAGAGCGAGAAGGGCCCCGUGUG
CUGGCGGAAGCGGGUGAAGAGCGAGUACAUGCGGCUGCGGCAGCUGAAGCGGUUCCGGCGGGCCG
ACGAGGUGAAGAGCAUGUUCAGCAGCAACCGGCAGAAGAUCCUGGAGCGGACCGAGAUCCUGAAC
CAGGAGUGGAAGCAGCGGCGAAUCCAGCCCGUGCACAUCCUGACCAGCGUGAGCAGCCUGCGGGG
CACCCGGGAGUGCAGCGUGACCAGCGACCUGGACUUCCCCACCCAGGUGAUCCCCCUAAAGACCC
UGAACGCCGUGGCCAGCGUGCCCAUCAUGUACAGCUGGAGCCCCCUGCAGCAGAACUUCAUGGUG
GAGGACGAGACCGUGCUGCACAACAUCCCCUACAUGGGCGACGAGGUGCUGGACCAGGACGGCAC
CUUCAUCGAGGAGCUGAUCAAGAACUACGACGGCAAGGUGCACGGCGACCGGGAGUGCGGCUUCA
UCAACGACGAGAUCUUCGUGGAGCUGGUGAACGCCCUGGGCCAGUACAACGACGACGACGACGAC
GACGACGGCGACGACCCCGAGGAGCGGGAGGAGAAGCAGAAGGACCUGGAGGACCACCGGGACGA
CAAGGAGAGCCGGCCCCCCCGGAAGUUCCCCAGCGACAAGAUCUUCGAGGCCAUCAGCAGCAUGU
UCCCCGACAAGGGCACCGCCGAGGAGCUGAAGGAGAAGUACAAGGAGCUGACCGAGCAGCAGCUG
CCCGGCGCCCUGCCCCCCGAGUGCACCCCCAACAUCGACGGCCCCAACGCCAAGAGCGUGCAGCG
GGAGCAGAGCCUGCACAGCUUCCACACCCUGUUCUGCCGGCGGUGCUUCAAGUACGACUGCUUCC
UGCACCCCUUCCACGCCACCCCCAACACCUACAAGCGGAAGAACACCGAGACCGCCCUGGACAAC
AAGCCCUGCGGCCCCCAGUGCUACCAGCACCUGGAGGGCGCCAAGGAGUUCGCCGCCGCCCUGAC
CGCCGAGCGGAUCAAGACCCCCCCCAAGCGGCCCGGCGGCCGGCGGCGGGGCCGGCUGCCCAACA
ACAGCAGCCGGCCCAGCACCCCCACCAUCAACGUGCUGGAGAGCAAGGACACCGACAGCGACCGG
GAGGCCGGCACCGAGACCGGCGGCGAGAACAACGACAAGGAGGAGGAGGAGAAGAAGGACGAGAC
CAGCAGCAGCAGCGAGGCCAACAGCCGGUGCCAGACCCCCAUCAAGAUGAAGCCCAACAUCGAGC
CCCCCGAGAACGUGGAGUGGAGCGGCGCCGAGGCCAGCAUGUUCCGGGUGCUGAUCGGCACCUAC
UACGACAACUUCUGCGCCAUCGCCCGGCUGAUCGGCACCAAGACCUGCCGGCAGGUGUACGAGUU
CCGGGUGAAGGAGAGCAGCAUCAUCGCCCCCGCCCCCGCCGAGGACGUGGACACCCCCCCCCGGA
AGAAGAAGCGGAAGCACCGGCUGUGGGCCGCCCACUGCCGGAAGAUCCAGCUGAAGAAGGACGGC
AGCAGCAACCACGUGUACAACUACCAGCCCUGCGACCACCCCCGGCAGCCCUGCGACAGCAGCUG
CCCCUGCGUGAUCGCCCAGAACUUCUGCGAGAAGUUCUGCCAGUGCAGCAGCGAGUGCCAGAACC
GGUUCCCCGGCUGCCGGUGCAAGGCCCAGUGCAACACCAAGCAGUGCCCCUGCUACCUGGCCGUG
CGGGAGUGCGACCCCGACCUGUGCCUGACCUGCGGCGCCGCCGACCACUGGGACAGCAAGAACGU
GAGCUGCAAGAACUGCAGCAUCCAGCGGGGCAGCAAGAAGCACCUGCUGCUGGCCCCCAGCGACG
UGGCCGGCUGGGGCAUCUUCAUCAAGGACCCCGUGCAGAAGAACGAGUUCAUCAGCGAGUACUGC
GGCGAGAUCAUCAGCCAGGACGAGGCCGACCGGCGGGGCAAGGUGUACGACAAGUACAUGUGCAG
CUUCCUGUUCAACCUGAACAACGACUUCGUGGUGGACGCCACCCGGAAGGGCAACAAGAUCCGGU
UCGCCAACCACAGCGUGAACCCCAACUGCUACGCCAAGGUGAUGAUGGUGAACGGCGACCACCGG
AUCGGCAUCUUCGCCAAGCGGGCCAUCCAGACCGGCGAGGAGCUGUUCUUCGACUACCGGUACAG
CCAGGCCGACGCCCUGAAGUACGUGGGCAUCGAGCGGGAGAUGGAGAUCCCCAGCACCGGCGGCA
GCGGCGGCAGCGGCGGCAGCGGCGGCAGCGGCGGCAGCGGCCGACCCGACAAGAAGUACAGCAUC
GGCCUGGCCAUCGGCACCAACAGCGUGGGCUGGGCCGUGAUCACCGACGAGUACAAGGUGCCCAG
CAAGAAGUUCAAGGUGCUGGGCAACACCGACCGGCACAGCAUCAAGAAGAACCUGAUCGGCGCCC
UGCUGUUCGACAGCGGCGAGACCGCCGAGGCCACCCGGCUGAAGCGGACCGCCCGGCGGCGGUAC
ACCCGGCGGAAGAACCGGAUCUGCUACCUGCAGGAGAUCUUCAGCAACGAGAUGGCCAAGGUGGA
CGACAGCUUCUUCCACCGGCUGGAGGAGAGCUUCCUGGUGGAGGAGGACAAGAAGCACGAGCGGC
ACCCCAUCUUCGGCAACAUCGUGGACGAGGUGGCCUACCACGAGAAGUACCCCACCAUCUACCAC
CUGCGGAAGAAGCUGGUGGACAGCACCGACAAGGCCGACCUGCGGCUGAUCUACCUGGCCCUGGC
CCACAUGAUCAAGUUCCGGGGCCACUUCCUGAUCGAGGGCGACCUGAACCCCGACAACAGCGACG
UGGACAAGCUGUUCAUCCAGCUGGUGCAGACCUACAACCAGCUGUUCGAGGAGAACCCCAUCAAC
GCCAGCGGCGUGGACGCCAAGGCCAUCCUGAGCGCCCGGCUGAGCAAGAGCCGGCGGCUGGAGAA
CCUGAUCGCCCAGCUGCCCGGCGAGAAGAAGAACGGCCUGUUCGGCAACCUGAUCGCCCUGAGCC
UGGGCCUGACCCCCAACUUCAAGAGCAACUUCGACCUGGCCGAGGACGCCAAGCUGCAGCUGAGC
AAGGACACCUACGACGACGACCUGGACAACCUGCUGGCCCAGAUCGGCGACCAGUACGCCGACCU
GUUCCUGGCCGCCAAGAACCUGAGCGACGCCAUCCUGCUGAGCGACAUCCUGCGGGUGAACACCG
AGAUCACCAAGGCCCCCCUGAGCGCCAGCAUGAUCAAGCGGUACGACGAGCACCACCAGGACCUG
ACCCUGCUGAAGGCCCUGGUGCGGCAGCAGCUGCCCGAGAAGUACAAGGAGAUCUUCUUCGACCA
GAGCAAGAACGGCUACGCCGGCUACAUCGACGGCGGCGCCAGCCAGGAGGAGUUCUACAAGUUCA
UCAAGCCCAUCCUGGAGAAGAUGGACGGCACCGAGGAGCUGCUGGUGAAGCUGAACCGGGAGGAC
CUGCUGCGGAAGCAGCGGACCUUCGACAACGGCAGCAUCCCCCACCAGAUCCACCUGGGCGAGCU
GCACGCCAUCCUGCGGCGGCAGGAGGACUUCUACCCCUUCCUGAAGGACAACCGGGAGAAGAUCG
AGAAGAUCCUGACCUUCCGGAUCCCCUACUACGUGGGCCCCCUGGCCCGGGGCAACAGCCGGUUC
GCCUGGAUGACCCGGAAAUCCGAGGAGACCAUCACCCCCUGGAACUUCGAGGAGGUGGUGGACAA
GGGCGCCAGCGCCCAGAGCUUCAUCGAGCGGAUGACCAACUUCGACAAGAACCUGCCCAACGAGA
AGGUGCUGCCCAAGCACAGCCUGCUGUACGAGUACUUCACCGUGUACAACGAGCUGACCAAGGUG
AAGUACGUGACCGAGGGCAUGCGGAAGCCCGCCUUCCUGAGCGGCGAGCAGAAGAAGGCCAUCGU
GGACCUGCUGUUCAAGACCAACCGGAAGGUGACCGUGAAGCAGCUGAAGGAGGACUACUUCAAGA
AGAUCGAGUGCUUCGACAGCGUGGAGAUCAGCGGCGUGGAGGACCGGUUCAACGCCAGCCUGGGC
ACCUACCACGACCUGCUGAAGAUCAUCAAGGACAAGGACUUCCUGGACAACGAGGAGAACGAGGA
CAUCCUGGAGGACAUCGUGCUGACCCUGACCCUGUUCGAGGACCGGGAGAUGAUCGAGGAGCGGC
UGAAAACCUACGCCCACCUGUUCGACGACAAGGUGAUGAAGCAGCUGAAGCGGCGGCGGUACACC
GGCUGGGGCCGGCUGAGCCGGAAGCUGAUCAACGGCAUCCGGGACAAGCAGAGCGGCAAGACCAU
CCUGGACUUCCUGAAAUCCGACGGCUUCGCCAACCGGAACUUCAUGCAGCUGAUCCACGACGACA
GCCUGACCUUCAAGGAGGACAUCCAGAAGGCCCAGGUGAGCGGCCAGGGCGACAGCCUGCACGAG
CACAUCGCCAACCUGGCCGGCAGCCCCGCCAUCAAGAAGGGCAUCCUGCAGACCGUGAAGGUGGU
GGACGAGCUGGUGAAGGUGAUGGGCCGGCACAAGCCCGAGAACAUCGUGAUCGAGAUGGCCCGGG
AGAACCAGACCACCCAGAAGGGCCAGAAGAACAGCCGGGAGCGGAUGAAGCGGAUCGAGGAGGGC
AUCAAGGAGCUGGGCAGCCAGAUCCUGAAGGAGCACCCCGUGGAGAACACCCAGCUGCAGAACGA
GAAGCUGUACCUGUACUACCUGCAGAACGGCCGGGACAUGUACGUGGACCAGGAGCUGGACAUCA
ACCGGCUGAGCGACUACGACGUGGCCGCCAUCGUGCCCCAGAGCUUCCUGAAGGACGACAGCAUC
GACAACAAGGUGCUGACCCGGAGCGACAAGGCCCGGGGCAAGAGCGACAACGUGCCCAGCGAGGA
GGUGGUGAAGAAGAUGAAGAACUACUGGCGGCAGCUGCUGAACGCCAAGCUGAUCACCCAGCGGA
AGUUCGACAACCUGACCAAGGCCGAGCGGGGCGGCCUGAGCGAGCUGGACAAGGCCGGCUUCAUC
AAGCGGCAGCUGGUGGAGACCCGGCAGAUCACCAAGCACGUGGCCCAGAUCCUGGACAGCCGGAU
GAACACCAAGUACGACGAGAACGACAAGCUGAUCCGGGAGGUGAAGGUGAUCACCCUGAAAUCCA
AGCUGGUGAGCGACUUCCGGAAGGACUUCCAGUUCUACAAGGUGCGGGAGAUCAACAACUACCAC
CACGCCCACGACGCCUACCUGAACGCCGUGGUGGGCACCGCCCUGAUCAAGAAGUACCCCAAGCU
GGAGAGCGAGUUCGUGUACGGCGACUACAAGGUGUACGACGUGCGGAAGAUGAUCGCCAAGAGCG
AGCAGGAGAUCGGCAAGGCCACCGCCAAGUACUUCUUCUACAGCAACAUCAUGAACUUCUUCAAG
ACCGAGAUCACCCUGGCCAACGGCGAGAUCCGGAAGCGGCCCCUGAUCGAGACCAACGGCGAGAC
CGGCGAGAUCGUGUGGGACAAGGGCCGGGACUUCGCCACCGUGCGGAAGGUGCUGAGCAUGCCCC
AGGUGAACAUCGUGAAGAAAACCGAGGUGCAGACCGGCGGCUUCAGCAAGGAGAGCAUCCUGCCC
AAGCGGAACAGCGACAAGCUGAUCGCCCGGAAGAAGGACUGGGACCCCAAGAAGUACGGCGGCUU
CGACAGCCCCACCGUGGCCUACAGCGUGCUGGUGGUGGCCAAGGUGGAGAAGGGCAAGAGCAAGA
AGCUGAAAUCCGUGAAGGAGCUGCUGGGCAUCACCAUCAUGGAGCGGAGCAGCUUCGAGAAGAAC
CCCAUCGACUUCCUGGAGGCCAAGGGCUACAAGGAGGUGAAGAAGGACCUGAUCAUCAAGCUGCC
CAAGUACAGCCUGUUCGAGCUGGAGAACGGCCGGAAGCGGAUGCUGGCCAGCGCCGGCGAGCUGC
AGAAGGGCAACGAGCUGGCCCUGCCCAGCAAGUACGUGAACUUCCUGUACCUGGCCAGCCACUAC
GAGAAGCUGAAGGGCAGCCCCGAGGACAACGAGCAGAAGCAGCUGUUCGUGGAGCAGCACAAGCA
CUACCUGGACGAGAUCAUCGAGCAGAUCAGCGAGUUCAGCAAGCGGGUGAUCCUGGCCGACGCCA
ACCUGGACAAGGUGCUGAGCGCCUACAACAAGCACCGGGACAAGCCCAUCCGGGAGCAGGCCGAG
AACAUCAUCCACCUGUUCACCCUGACCAACCUGGGCGCCCCCGCCGCCUUCAAGUACUUCGACAC
CACCAUCGACCGGAAGCGGUACACCAGCACCAAGGAGGUGCUGGACGCCACCCUGAUCCACCAGA
GCAUCACCGGCCUGUACGAGACCCGGAUCGACCUGAGCCAGCUGGGCGGCGACAGCGGCGGCAAG
CGGCCCGCCGCCACCAAGAAGGCCGGCCAGGCCAAGAAGAAGAAGGGCAGCUACCCCUACGACGU
GCCCGACUACGCCUGAGCGGCCGCUUAAUUAAGCUGCCUUCUGCGGGGCUUGCCUUCUGGCCAUG
CCCUUCUUCUCUCCCUUGCACCUGUACCUCUUGGUCUUUGAAUAAAGCCUGAGUAGGAAGUCUAG
AAAAAAAAAAAAAAAAAAAAAAAAAAAAAAAAAAAAAAAAAAAAAAAAAAAAAAAAAAAAAAAAA
AAAAAAAAAAAAAAA
>Nucleotide sequence of the Effector Fusion
Protein of dCas9-HDAC8
SEQ ID NO: 170
AGGAAAUAAGAGAGAAAAGAAGAGUAAGAAGAAAUAUAAGAGCCACCAUGGCCCCCAAGAAGAAG
CGGAAGGUGGGCAUCCACGGCGUGCCCGCCGCCGACAAGAAGUACAGCAUCGGCCUGGCCAUCGG
CACCAACAGCGUGGGCUGGGCCGUGAUCACCGACGAGUACAAGGUGCCCAGCAAGAAGUUCAAGG
UGCUGGGCAACACCGACCGGCACAGCAUCAAGAAGAACCUGAUCGGCGCCCUGCUGUUCGACAGC
GGCGAGACCGCCGAGGCCACCCGGCUGAAGCGGACCGCCCGGCGGCGGUACACCCGGCGGAAGAA
CCGGAUCUGCUACCUGCAGGAGAUCUUCAGCAACGAGAUGGCCAAGGUGGACGACAGCUUCUUCC
ACCGGCUGGAGGAGAGCUUCCUGGUGGAGGAGGACAAGAAGCACGAGCGGCACCCCAUCUUCGGC
AACAUCGUGGACGAGGUGGCCUACCACGAGAAGUACCCCACCAUCUACCACCUGCGGAAGAAGCU
GGUGGACAGCACCGACAAGGCCGACCUGCGGCUGAUCUACCUGGCCCUGGCCCACAUGAUCAAGU
UCCGGGGCCACUUCCUGAUCGAGGGCGACCUGAACCCCGACAACAGCGACGUGGACAAGCUGUUC
AUCCAGCUGGUGCAGACCUACAACCAGCUGUUCGAGGAGAACCCCAUCAACGCCAGCGGCGUGGA
CGCCAAGGCCAUCCUGAGCGCCCGGCUGAGCAAGAGCCGGCGGCUGGAGAACCUGAUCGCCCAGC
UGCCCGGCGAGAAGAAGAACGGCCUGUUCGGCAACCUGAUCGCCCUGAGCCUGGGCCUGACCCCC
AACUUCAAGAGCAACUUCGACCUGGCCGAGGACGCCAAGCUGCAGCUGAGCAAGGACACCUACGA
CGACGACCUGGACAACCUGCUGGCCCAGAUCGGCGACCAGUACGCCGACCUGUUCCUGGCCGCCA
AGAACCUGAGCGACGCCAUCCUGCUGAGCGACAUCCUGCGGGUGAACACCGAGAUCACCAAGGCC
CCCCUGAGCGCCAGCAUGAUCAAGCGGUACGACGAGCACCACCAGGACCUGACCCUGCUGAAGGC
CCUGGUGCGGCAGCAGCUGCCCGAGAAGUACAAGGAGAUCUUCUUCGACCAGAGCAAGAACGGCU
ACGCCGGCUACAUCGACGGCGGCGCCAGCCAGGAGGAGUUCUACAAGUUCAUCAAGCCCAUCCUG
GAGAAGAUGGACGGCACCGAGGAGCUGCUGGUGAAGCUGAACCGGGAGGACCUGCUGCGGAAGCA
GCGGACCUUCGACAACGGCAGCAUCCCCCACCAGAUCCACCUGGGCGAGCUGCACGCCAUCCUGC
GGCGGCAGGAGGACUUCUACCCCUUCCUGAAGGACAACCGGGAGAAGAUCGAGAAGAUCCUGACC
UUCCGGAUCCCCUACUACGUGGGCCCCCUGGCCCGGGGCAACAGCCGGUUCGCCUGGAUGACCCG
GAAAUCCGAGGAGACCAUCACCCCCUGGAACUUCGAGGAGGUGGUGGACAAGGGCGCCAGCGCCC
AGAGCUUCAUCGAGCGGAUGACCAACUUCGACAAGAACCUGCCCAACGAGAAGGUGCUGCCCAAG
CACAGCCUGCUGUACGAGUACUUCACCGUGUACAACGAGCUGACCAAGGUGAAGUACGUGACCGA
GGGCAUGCGGAAGCCCGCCUUCCUGAGCGGCGAGCAGAAGAAGGCCAUCGUGGACCUGCUGUUCA
AGACCAACCGGAAGGUGACCGUGAAGCAGCUGAAGGAGGACUACUUCAAGAAGAUCGAGUGCUUC
GACAGCGUGGAGAUCAGCGGCGUGGAGGACCGGUUCAACGCCAGCCUGGGCACCUACCACGACCU
GCUGAAGAUCAUCAAGGACAAGGACUUCCUGGACAACGAGGAGAACGAGGACAUCCUGGAGGACA
UCGUGCUGACCCUGACCCUGUUCGAGGACCGGGAGAUGAUCGAGGAGCGGCUGAAAACCUACGCC
CACCUGUUCGACGACAAGGUGAUGAAGCAGCUGAAGCGGCGGCGGUACACCGGCUGGGGCCGGCU
GAGCCGGAAGCUGAUCAACGGCAUCCGGGACAAGCAGAGCGGCAAGACCAUCCUGGACUUCCUGA
AAUCCGACGGCUUCGCCAACCGGAACUUCAUGCAGCUGAUCCACGACGACAGCCUGACCUUCAAG
GAGGACAUCCAGAAGGCCCAGGUGAGCGGCCAGGGCGACAGCCUGCACGAGCACAUCGCCAACCU
GGCCGGCAGCCCCGCCAUCAAGAAGGGCAUCCUGCAGACCGUGAAGGUGGUGGACGAGCUGGUGA
AGGUGAUGGGCCGGCACAAGCCCGAGAACAUCGUGAUCGAGAUGGCCCGGGAGAACCAGACCACC
CAGAAGGGCCAGAAGAACAGCCGGGAGCGGAUGAAGCGGAUCGAGGAGGGCAUCAAGGAGCUGGG
CAGCCAGAUCCUGAAGGAGCACCCCGUGGAGAACACCCAGCUGCAGAACGAGAAGCUGUACCUGU
ACUACCUGCAGAACGGCCGGGACAUGUACGUGGACCAGGAGCUGGACAUCAACCGGCUGAGCGAC
UACGACGUGGCCGCCAUCGUGCCCCAGAGCUUCCUGAAGGACGACAGCAUCGACAACAAGGUGCU
GACCCGGAGCGACAAGGCCCGGGGCAAGAGCGACAACGUGCCCAGCGAGGAGGUGGUGAAGAAGA
UGAAGAACUACUGGCGGCAGCUGCUGAACGCCAAGCUGAUCACCCAGCGGAAGUUCGACAACCUG
ACCAAGGCCGAGCGGGGCGGCCUGAGCGAGCUGGACAAGGCCGGCUUCAUCAAGCGGCAGCUGGU
GGAGACCCGGCAGAUCACCAAGCACGUGGCCCAGAUCCUGGACAGCCGGAUGAACACCAAGUACG
ACGAGAACGACAAGCUGAUCCGGGAGGUGAAGGUGAUCACCCUGAAAUCCAAGCUGGUGAGCGAC
UUCCGGAAGGACUUCCAGUUCUACAAGGUGCGGGAGAUCAACAACUACCACCACGCCCACGACGC
CUACCUGAACGCCGUGGUGGGCACCGCCCUGAUCAAGAAGUACCCCAAGCUGGAGAGCGAGUUCG
UGUACGGCGACUACAAGGUGUACGACGUGCGGAAGAUGAUCGCCAAGAGCGAGCAGGAGAUCGGC
AAGGCCACCGCCAAGUACUUCUUCUACAGCAACAUCAUGAACUUCUUCAAGACCGAGAUCACCCU
GGCCAACGGCGAGAUCCGGAAGCGGCCCCUGAUCGAGACCAACGGCGAGACCGGCGAGAUCGUGU
GGGACAAGGGCCGGGACUUCGCCACCGUGCGGAAGGUGCUGAGCAUGCCCCAGGUGAACAUCGUG
AAGAAAACCGAGGUGCAGACCGGCGGCUUCAGCAAGGAGAGCAUCCUGCCCAAGCGGAACAGCGA
CAAGCUGAUCGCCCGGAAGAAGGACUGGGACCCCAAGAAGUACGGCGGCUUCGACAGCCCCACCG
UGGCCUACAGCGUGCUGGUGGUGGCCAAGGUGGAGAAGGGCAAGAGCAAGAAGCUGAAAUCCGUG
AAGGAGCUGCUGGGCAUCACCAUCAUGGAGCGGAGCAGCUUCGAGAAGAACCCCAUCGACUUCCU
GGAGGCCAAGGGCUACAAGGAGGUGAAGAAGGACCUGAUCAUCAAGCUGCCCAAGUACAGCCUGU
UCGAGCUGGAGAACGGCCGGAAGCGGAUGCUGGCCAGCGCCGGCGAGCUGCAGAAGGGCAACGAG
CUGGCCCUGCCCAGCAAGUACGUGAACUUCCUGUACCUGGCCAGCCACUACGAGAAGCUGAAGGG
CAGCCCCGAGGACAACGAGCAGAAGCAGCUGUUCGUGGAGCAGCACAAGCACUACCUGGACGAGA
UCAUCGAGCAGAUCAGCGAGUUCAGCAAGCGGGUGAUCCUGGCCGACGCCAACCUGGACAAGGUG
CUGAGCGCCUACAACAAGCACCGGGACAAGCCCAUCCGGGAGCAGGCCGAGAACAUCAUCCACCU
GUUCACCCUGACCAACCUGGGCGCCCCCGCCGCCUUCAAGUACUUCGACACCACCAUCGACCGGA
AGCGGUACACCAGCACCAAGGAGGUGCUGGACGCCACCCUGAUCCACCAGAGCAUCACCGGCCUG
UACGAGACCCGGAUCGACCUGAGCCAGCUGGGCGGCGACAGCGGCGGCAAGCGGCCCGCCGCCAC
CAAGAAGGCCGGCCAGGCCAAGAAGAAGAAGUCGGGCGGGGGUGGCUCAGAGGAGCCCGAGGAGC
CCGCCGAUAGCGGACAAUCUCUGGUGCCCGUCUACAUCUACAGCCCCGAAUAUGUGAGCAUGUGU
GAUUCCCUCGCCAAGAUCCCUAAGAGAGCCAGCAUGGUGCAUUCUCUGAUCGAGGCCUACGCUCU
GCAUAAGCAAAUGAGGAUCGUGAAGCCCAAGGUCGCCAGCAUGGAAGAGAUGGCCACCUUUCACA
CCGAUGCCUACCUCCAACAUCUCCAGAAGGUGUCCCAAGAGGGCGACGACGACCACCCCGACUCC
AUUGAGUACGGACUGGGCUAUGAUUGCCCCGCCACCGAGGGCAUCUUUGACUAUGCCGCCGCUAU
CGGCGGAGCUACCAUCACAGCCGCCCAGUGUCUGAUUGAUGGCAUGUGCAAGGUCGCCAUCAACU
GGUCCGGAGGCUGGCAUCAUGCCAAGAAGGAUGAGGCCUCCGGCUUCUGUUAUCUGAAUGACGCC
GUGCUGGGCAUUCUGAGACUGAGGAGGAAAUUCGAGAGGAUUCUGUACGUGGAUCUGGAUCUGCA
UCACGGAGAUGGAGUCGAAGAUGCCUUCAGCUUCACCAGCAAGGUGAUGACAGUCUCUCUGCACA
AGUUCUCCCCCGGCUUCUUUCCCGGAACCGGCGACGUGUCCGACGUGGGACUGGGCAAGGGAAGG
UACUACAGCGUGAACGUGCCCAUUCAAGACGGCAUCCAAGACGAGAAGUACUACCAGAUCUGCGA
GUCCGUGCUCAAGGAGGUCUACCAAGCCUUCAAUCCUAAGGCUGUCGUGCUCCAACUGGGAGCUG
AUACCAUUGCUGGCGAUCCCAUGUGCAGCUUCAAUAUGACACCCGUCGGAAUCGGCAAGUGCCUC
AAGUACAUCCUCCAGUGGCAGCUCGCCACCCUCAUUCUCGGAGGAGGCGGAUACAAUCUGGCUAA
UACCGCCAGAUGCUGGACCUAUCUGACCGGCGUGAUUCUGGGCAAAACACUGAGCAGCGAAAUCC
CCGACCACGAGUUUUUCACCGCUUACGGCCCCGACUACGUGCUGGAGAUCACCCCCAGCUGCAGA
CCCGAUAGAAACGAACCCCAUAGAAUCCAGCAAAUUCUGAACUAUAUCAAGGGCAACCUCAAGCA
CGUCGUGGGAGGUGGCGGAUCGGGAAAGCGGCCCGCCGCCACCAAGAAGGCCGGUCAGGCCAAGA
AGAAGAAGGGCAGCUACCCCUACGACGUGCCCGACUACGCCUGAGCGGCCGCUUAAUUAAGCUGC
CUUCUGCGGGGCUUGCCUUCUGGCCAUGCCCUUCUUCUCUCCCUUGCACCUGUACCUCUUGGUCU
UUGAAUAAAGCCUGAGUAGGAAGUCUAGAAAAAAAAAAAAAAAAAAAAAAAAAAAAAAAAAAAAA
AAAAAAAAAAAAAAAAAAAAAAAAAAAAAAAAAAAAAAAAAAA
>Amino acid Sequence of DNA Binding Domain
of the Effector Fusion Protein of dCas9-MQ1
SEQ ID NO: 171
DKKYSIGLAIGTNSVGWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYD
EHHQDLTLLKALVRQQLPEKYKEIFFDQSKNGYAGYIDGGASQEEFYKFIKPILEKMDGTEELLV
KLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVAAIVPQSF
LKDDSIDNKVLTRSDKARGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSN
IMNFFKTEITLANGEIRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSKKLKSVKELLGITIMER
SSFEKNPIDFLEAKGYKEVKKDLIIKLPKYSLFELENGRKRMLASAGELQKGNELALPSKYVNFL
YLASHYEKLKGSPEDNEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQSITGLYETRIDLSQLG
GD

### EQUIVALENTS

Those skilled in the art will recognize, or be able to ascertain using no more than routine experimentation, many equivalents to the specific embodiments of the present invention described herein. Such equivalents are intended to be encompassed by the following claims.

We claim:

1. A site-specific epigenetic modifying agent, comprising at least one site-specific targeting moiety which targets a target sequence within an expression control region of human CXCL9, CXCL10, or CXCL11.

2. The epigenetic modifying agent of claim 1, wherein the target sequence is within a human chromosomal region selected from the group consisting of chr4: 76928543-76931536, chr4: 76944524-76948668, chr4: 76957098-76962383, chr4: 76949523-76949873, chr4: 76932906-76933929, chr4: 76916900-76917300, chr4: 76988761-76989260, chr4: 77008274-77008727, and chr4: 76957852-76958551.

3. The epigenetic modifying agent of claim 1, wherein the target sequence is located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb from a human chromosomal region selected from the group consisting of chr4: 76958201-76958221, chr4: 76949758-76949778, chr4: 76933245-76933265, chr4: 76932967-76932987, chr4: 76957115-76957132, chr4: 76944734-76944751, chr4: 76944656-76944673, and chr4: 76933040-76933057.

4. The epigenetic modifying agent of any one of claims 1-3, wherein the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

5. The site specific epigenetic modifying agent any one of claims 1-4, comprising at least two site specific targeting moieties, each of which independently targets CXCL9, CXCL10 or CXCL11.

6. The epigenetic modifying agent of any one of claims 1-5, wherein the site-specific targeting moiety comprises a polymeric molecule.

7. The epigenetic modifying agent of claim 6, wherein the polymeric molecule comprises a polyamide.

8. The epigenetic modifying agent of claim 6 or 7, wherein the polymeric molecule comprises a polynucleotide.

9. The epigenetic modifying agent of any one of claims 1-8, wherein the expression control region comprises at least one specific transcriptional control element for CXCL9, CXCL10, or CXCL11.

10. The site specific epigenetic modifying agent of claim 1 comprising at least two specific transcriptional control elements, each of which independently reduces transcription of CXCL9, CXCL10 or CXCL11.

11. The epigenetic modifying agent of claim 9 or 10, wherein the transcriptional control element comprises a promoter for CXCL9, CXCL10, or CXCL11.

12. The epigenetic modifying agent of any one of claims 9-11, wherein the transcriptional control element comprises an enhancer for CXCL9, CXCL10, or CXCL11.

13. The epigenetic modifying agent of any one of claims 1-9, wherein the site-specific targeting moiety comprises a nucleotide sequence having at least 85%, 90%, 95%, 96%, 97%, 98% or 99% nucleotide identity to the entire nucleotide sequence of any of the nucleotide sequences in any one of Table 3 or Table 5.

14. The epigenetic modifying agent of any one of claims 6-13, wherein the polymeric molecule comprises a polynucleotide encoding a Cas or dCas polypeptide, or a fragment thereof, and a polynucleotide encoding a guide RNA that specifically binds to the expression control region of CXCL9, CXCL10, or CXCL11; or a DNA-binding domain of a Transcription activator-like effector (TALE) polypeptide or a zinc finger (ZFP) polypeptide, or fragment thereof, that specifically binds to the expression control region of CXCL9, CXCL10, or CXCL11.

15. The epigenetic modifying agent of claim 14, wherein the DNA-binding domain of the ZFP or TALE polypeptide comprises an amino acid sequence having at least about 85%, 90%, 95%, 96%, 97%, 98% or 99% amino acid identity to the entire amino acid sequence of any one of the amino acid sequences listed in Table 3 or Table 5.

16. The epigenetic modifying agent of any one of claims 1-15, wherein the expression control region comprises
1) one or more CXCL9-associated anchor sequences within an anchor sequence-mediated conjunction comprising a first and a second CXCL9-associated anchor sequence;
2) one or more CXCL10-associated anchor sequences within an anchor sequence-mediated conjunction comprising a first and a second CXCL10-associated anchor sequence; or
3) one or more CXCL11-associated anchor sequences within an anchor sequence-mediated conjunction comprising a first and a second CXCL11-associated anchor sequence.

17. The epigenetic modifying agent of any one of claims 1-16, wherein the expression control region comprises one or more CCCTC-binding factor (CTCF) binding motifs.

18. The epigenetic modifying agent of claim 16 or 17, wherein the anchor sequence-mediated conjunction comprises one or more transcriptional control elements internal to the conjunction.

19. The epigenetic modifying agent of claim 16 or 17, wherein the anchor sequence-mediated conjunction comprises one or more transcriptional control elements external to the conjunction.

20. The epigenetic modifying agent of any one of claims 16-19, wherein the first and/or the second anchor sequence is located within about 500 kb of the transcriptional control element.

21. The epigenetic modifying agent of claim 20, wherein the first and/or the second anchor sequence is located within about 300 kb of the transcriptional control element.

22. The epigenetic modifying agent of claim 21, wherein the first and/or the second anchor sequence is located within 10 kb of the transcriptional control element.

23. The epigenetic modifying agent any one of claims 6-22, wherein the polymeric molecule comprises a polynucleotide encoding a DNA-binding domain, or fragment thereof, of a zinc finger polypeptide (ZFP) or a transcription activator-like effector (TALE) polypeptide that specifically binds to the expression control region of CXCL9, CXCL10, or CXCL11.

24. The epigenetic modifying agent of claim 20, wherein the DNA-binding domain of the ZFP or TALE polypeptide comprises an amino acid sequence having at least about 85%, 90%, 95%, 96%, 97%, 98% or 99% amino acid identity to the entire amino acid sequence of any one of the amino acid sequences listed in Table 3 or Table 5.

25. The epigenetic modifying agent of any one of claims 1-24, wherein the epigenetic modifying agent comprises a nucleotide modification.

26. The epigenetic modifying agent any one of claims 6-24, wherein the polymeric molecule comprises a peptide nucleic acid (PNA).

27. A nucleic acid molecule, wherein the nucleic acid molecule encodes the epigenetic modifying agent of any one of claims 1-26.

28. A vector comprising the epigenetic modifying agent of any one of claims 1-27.

29. The vector of claim 28, wherein the vector is a viral expression vector.

30. A cell comprising the epigenetic modifying agent of any one of claims 1-26 or the vector of claim 28 or 29.

31. A composition comprising the epigenetic modifying agent of any one of claims 1-26.

32. The composition of claim 31, wherein the composition comprises at least two epigenetic modifying agents, each of which independently targets an expression control region of one of CXCL9, CXCL10, or CXCL11.

33. The composition of claim 31 or 32, wherein the composition comprises at least three epigenetic modifying agent, each of which targets an expression control region of one of CXCL9, CXCL10, or CXCL11.

34. The composition of claim 31, wherein the composition comprises an epigenetic modifying agent which targets the expression control region of at least two of CXCL9, CXCL10, or CXCL11.

35. The composition of any one of claims 31-34, wherein the composition comprises a pharmaceutical composition.

36. The composition of claim 35, wherein the pharmaceutical composition comprises a lipid formulation.

37. The composition of claim 36, wherein the lipid formulation comprises one or more cationic lipids, one or more non-cationic lipids, one or more cholesterol-based lipids, or one or more PEG-modified lipids, or combinations of any of the foregoing.

38. The composition of claim 35, wherein the pharmaceutical composition comprises a lipid nanoparticle.

39. An epigenetic modifying agent, comprising at least one nucleic acid molecule encoding a fusion protein, the fusion protein comprising a site-specific targeting moiety which targets a target sequence within an expression control region of human CXCL9, CXCL10, or CXCL11, and an effector molecule.

40. The epigenetic modifying agent of claim 39, wherein the target sequence is within a human chromosomal region selected from the group consisting of chr4: 76928543-76931536, chr4: 76944524-76948668, chr4: 76957098-76962383, chr4: 76949523-76949873, chr4: 76932906-76933929, chr4: 76916900-76917300, chr4: 76988761-76989260, chr4: 77008274-77008727, and chr4: 76957852-76958551.

41. The epigenetic modifying agent of claim 39, wherein target sequence is located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb from a human chromosomal region selected from the group consisting of chr4: 76958201-76958221, chr4: 76949758-76949778, chr4: 76933245-76933265, chr4: 76932967-76932987, chr4: 76957115-76957132, chr4: 76944734-76944751, chr4: 76944656-76944673, and chr4: 76933040-76933057.

42. The epigenetic modifying agent of any one of claims 38-41, wherein the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

43. The epigenetic modifying agent of claim 38-42, wherein the site-specific targeting moiety comprises a polynucleotide encoding a Cas or dCas polypeptide, or a fragment thereof, and a polynucleotide encoding a guide RNA that specifically binds to the expression control region of CXCL9, CXCL10, or CXCL11; or a DNA-binding domain of a Transcription activator-like effector (TALE) polypeptide or a zinc finger (ZFP) polypeptide, or fragment thereof, that specifically binds to the expression control region of CXCL9, CXCL10, or CXCL11.

44. The epigenetic modifying agent of claim 43, wherein the DNA-binding domain of the zinc finger polypeptide (ZFP) or TALE polypeptide comprises an amino acid sequence having at least 85%, 90%, 95%, 96%, 97%, 98% or 99% amino acid identity to the entire amino acid sequence of an amino acid sequence selected from the amino acid sequences listed in Table 3 or Table 5.

45. The epigenetic modifying agent of any one of claims 38-44, wherein the effector molecule comprises a polypeptide or a nucleic acid molecule encoding a polypeptide.

46. The epigenetic modifying agent of any one of claims 38-45, wherein the fusion protein comprises a peptide-nucleic acid fusion.

47. The epigenetic modifying agent of any one of claims 38-46, wherein the effector is selected from the group consisting of a nuclease, a physical blocker, an epigenetic recruiter, an epigenetic modifier, and combinations of any of the foregoing.

48. The epigenetic modifying agent of claim 47, wherein the effector is selected from the group consisting of euchromatic histone-lysine N-methyltransferase 2 (G9a), enhancer of zeste homolog 2 (EZH2), MQ1 domain (MQ1), Krüppel associated box (KRAB) transcriptional repression domain, and histone deacetylase 8 (HDAC8).

49. The epigenetic modifying agent of claim 48, wherein the effector is MQ1.

50. The epigenetic modifying agent of claim 49, wherein the effector comprises a CRISPR associated protein (Cas) polypeptide or nucleic acid molecule encoding the Cas polypeptide.

51. The epigenetic modifying agent of claim 50, wherein the Cas polypeptide is an enzymatically inactive Cas polypeptide.

52. The epigenetic modifying agent of claim 50 or 51, further comprising a catalytically active domain of human exonuclease 1 (hEXO1).

53. The epigenetic modifying agent of claim 47, wherein the epigenetic modifier comprises a transcriptional repressor.

54. The epigenetic modifying agent of claim 53, wherein the transcriptional repressor is selected from the group comprising euchromatic histone-lysine N-methyltransferase 2 (G9a), enhancer of zeste homolog 2 (EZH2), MQ1 domain (MQ1), Krüppel associated box (KRAB) transcriptional repression domain, and histone deacetylase 8 (HDAC8).

55. The epigenetic modifying agent of claim 54, wherein the transcriptional repressor is MQ1 domain.

56. The epigenetic modifying agent of claim 55, wherein the MQ1 domain comprises an amino acid sequence having at least about 85%, 90%, 95%, 96%, 97%, 98% or 99% amino acid identity to the entire amino acid sequence of SEQ ID NO: 79.

57. The epigenetic modifying agent of claim 55 or 56, wherein the transcriptional repressor comprises two, three, four, or five MQ1s.

58. The epigenetic modifying agent of claim 57, wherein the transcriptional repressor is a EZH2.

59. The epigenetic modifying agent of claim 58, wherein the EZH2 comprises an amino acid sequence having at least about 85%, 90%, 95%, 96%, 97%, 98% or 99% identity to the entire amino acid sequence of SEQ ID NO: 78.

60. The epigenetic modifying agent of claim 47, wherein the epigenetic modifier comprises a DNA methylase or a histone deacetylase.

61. The epigenetic modifying agent of claim 48, wherein the effector molecule comprises a Krüppel associated box (KRAB) transcriptional repression domain.

62. The epigenetic modifying agent of claim 61, wherein the KRAB comprises an amino acid sequence having at least about 85%, 90%, 95%, 96%, 97%, 98% or 99% identity to the entire amino acid sequence of SEQ ID NO: 297.

63. The epigenetic modifying agent of any one of claims 39-62, wherein the effector molecule comprises a Transcription activator-like effector nuclease (TALEN) polypeptide.

64. The epigenetic modifying agent of any one of claims 39-63, further comprising a second nucleic acid molecule encoding a second fusion protein, wherein
1) the second fusion protein comprises a second site-specific targeting moiety which targets a second expression control region of CXCL9, CXCL10 or CXCL11, which is the same or different than the target of the first site-specific targeting moiety;
2) the second fusion protein comprises a second site-specific targeting moiety which targets a second CXCL9 expression control region and a second effector molecule, wherein the second CXCL9 expression control region is different than the CXCL9 expression control region;
3) the second fusion protein comprises a second site-specific targeting moiety which targets a second CXCL10 expression control region and a second effector molecule, wherein the second CXCL10 expression control region is different than the CXCL10 expression control region; and/or
4) the second fusion protein comprises a second site-specific targeting moiety which targets a second CXCL11 expression control region and a second effector molecule, wherein the second CXCL11 expression control region is different than the CXCL11 expression control region.

65. The epigenetic modifying agent of claim 64, wherein the first nucleic acid molecule and the second nucleic acid molecule are located on the same nucleic acid molecule or on different nucleic acid molecules.

66. The epigenetic modifying agent of any of claims 39-65, wherein
1) the epigenetic modifying agent targets a CXCL9 expression control region comprising either a ZF target sequence comprising SEQ ID NOs: 422-425, or TALE target sequence comprising SEQ ID NOs: 429-432, or a combination thereof, and a second CXCL9 expression control region comprising either a ZF target sequence comprising SEQ ID NOs: 422-425, or TALE target sequence comprising SEQ ID NOs: 429-432, or a combination thereof; and/or
2) the epigenetic modifying agent targets a CXCL10 expression control region comprising either a ZF target sequence comprising SEQ ID NOs: 422-425, or TALE target sequence comprising SEQ ID NOs: 429-432, or a combination thereof, and a second CXCL10 expression control region comprising either a ZF target sequence comprising SEQ ID NOs: 422-425, or TALE target sequence comprising SEQ ID NOs: 429-432, or a combination thereof; and/or
3) the epigenetic modifying agent targets a CXCL11 expression control region comprising either a ZF target sequence comprising SEQ ID NOs: 422-425, or TALE target sequence comprising SEQ ID NOs: 429-432, or a combination thereof, a second CXCL11 expression control region comprising either a ZF target sequence comprising SEQ ID NOs: 422-425, or TALE target sequence comprising SEQ ID NOs: 429-432, or a combination thereof.

67. The epigenetic modifying agent of claim 65 or 66, wherein the second effector is different than the effector.

68. The epigenetic modifying agent of claim 67, wherein the second effector is the same as the effector.

69. The epigenetic modifying agent of any one of claims 65-68, wherein the fusion protein and the second fusion protein are operably linked.

70. The epigenetic modifying agent of any one of claims 39-69, wherein the fusion protein, and, optionally, the second fusion protein independently comprise an amino acid sequence that has at least about 85%, 90%, 95%, 96%, 97%, 98% or 99% amino acid sequence identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

71. The epigenetic modifying agent of any one of claims 39-70, wherein the fusion protein, and optionally, the second fusion protein, are independently encoded by a polynucleotide comprising a nucleotide sequence that has at least about 85%, 90%, 95%, 96%, 97%, 98% or 99% nucleotide sequence identity to the entire nucleotide sequence of a polynucleotide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

72. An epigenetic modifying agent, comprising a nucleic acid molecule encoding a fusion protein, wherein the fusion protein comprises an amino acid sequence having at least about 85%, 90%, 95%, 96%, 97%, 98% or 99% amino acid identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

73. The epigenetic modifying agent of claim 72, wherein the polypeptide is ZF05-KRAB.

74. The epigenetic modifying agent of claim 72, wherein the polypeptide is ZF07-KRAB.

75. The epigenetic modifying agent of claim 72, wherein the polypeptide is ZF16-KRAB.

76. The epigenetic modifying agent of claim 72, wherein the polypeptide is ZF24-KRAB.

77. The epigenetic modifying agent of claim 72, wherein the polypeptide is ZF05-MQ1.

78. The epigenetic modifying agent of claim 72, wherein the polypeptide is ZF07-MQ1.

79. The epigenetic modifying agent of claim 72, wherein the polypeptide is TAL04-KRAB.

80. The epigenetic modifying agent of claim 72, wherein the polypeptide is TAL08-KRAB.

81. The epigenetic modifying agent of claim 72, wherein the polypeptide is TAL09-KRAB.

82. The epigenetic modifying agent of claim 72, wherein the polypeptide is TAL14-KRAB.

83. An epigenetic modifying agent, comprising a nucleic acid molecule encoding two or more fusion proteins, wherein each fusion protein independently comprises an amino acid sequence having at least about 85%, 90%, 95%, 96%, 97%, 98% or 99% amino acid identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

84. A vector comprising a nucleic acid molecule encoding the epigenetic modifying agent of any one of claims 39-83.

85. The vector of claim 84, wherein the vector is a viral expression vector.

86. A cell comprising the epigenetic modifying agent of any one of claims 39-83 or the vector of claim 84 or 85.

87. The cell of claim 86, wherein the cell is an immune cell.

88. A composition comprising the epigenetic modifying agent of any one of claims 39-83.

89. The composition of claim 88, wherein the composition comprises at least one epigenetic modifying agent that targets an expression control region of CXCL9, CXCL10, or CXCL11.

90. The composition of claim 88 or 89, wherein the composition comprises at least two epigenetic modifying agent, each of which independently targets an expression control region of CXCL9, CXCL10, or CXCL11.

91. The composition of any one of claims 88-90, wherein the composition comprises at least three epigenetic modifying agent, each of which targets an expression control region of CXCL9, CXCL10, or CXCL11.

92. The composition of any one of claims 88-91, wherein the composition comprises a pharmaceutical composition.

93. The composition of claim 92, wherein the pharmaceutical composition comprises a lipid formulation.

94. The composition of claim 93, wherein the lipid formulation comprises one or more cationic lipids, one or more non-cationic lipids, one or more cholesterol-based lipids, or one or more PEG-modified lipids, or combinations of any of the foregoing.

95. The composition of claim 93, wherein the pharmaceutical composition comprises a lipid nanoparticle.

96. A method of reducing expression of CXCL9, CXCL10, or CXCL11 in a cell, the method comprising contacting the cell with at least one epigenetic modifying agent, each epigenetic modifying agent comprising at least one site-specific targeting moiety which targets a target sequence within an expression control region of human CXCL9, CXCL10, or CXCL11, and an effector molecule, thereby reducing expression of human CXCL9, CXCL10, or CXCL11 in the cell.

97. The method of claim 96, wherein the target sequence is within a human chromosomal region selected from the group consisting of chr4: 76928543-76931536, chr4: 76944524-76948668, chr4: 76957098-76962383, chr4: 76949523-76949873, chr4: 76932906-76933929, chr4: 76916900-76917300, chr4: 76988761-76989260, chr4: 77008274-77008727, and chr4: 76957852-76958551.

98. The method of claim 96, wherein the target sequence is located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb from a human chromosomal region selected from the group consisting of chr4: 76958201-76958221, chr4: 76949758-76949778, chr4: 76933245-76933265, chr4: 76932967-76932987, chr4: 76957115-76957132, chr4: 76944734-76944751, chr4: 76944656-76944673, and chr4: 76933040-76933057.

100. The method of any one of claims 96-98, wherein the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

101. The method of any one of claims 96-100, wherein the epigenetic modifying agent comprises at least two site specific targeting moieties, each of which independently targets CXCL9, CXCL10 or CXCL11.

102. The method of claim 96-101, wherein the expression of CXCL9, CXCL10, or CXCL11 in the cell is reduced by at least 30%, at least 35%, at least 40%, at least 45%, at least 50%, at least 55%, at least 60%, at least 65%, at least 70%, at least 75%, at least 80%, at least 85%, at least 90%, or at least 95% compared to prior to contacting the cell with the at least one epigenetic modifying agent.

103. The method of claim 96-102, wherein the expression of CXCL9, CXCL10, or CXCL11 in the cell is reduced between about 30% to about 100%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 35% to about 100%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 40% to about 100%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 100%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 100%, about 55% to about 95%, about 55% to about 90%, about 55% to about 85%, about 55% to about 80%, about 55% to about 75%, about 55% to about 70%, about 55% to about 65%, about 55% to about 60%, about 60% to about 100%, about 60% to about 95%, about 60% to about 90%, about 60% to about 85%, about 60% to about 80%, about 60% to about 75%, about 60% to about 70%, about 60% to about 65%, about 65% to about 100%, about 65% to about 95%, about 65% to about 90%, about 65% to about 85%, about 65% to about 80%, about 65% to about 75%, about 65% to about 70%, about 70% to about 100%, about 70% to about 95%, about 70% to about 90%, about 70% to about 85%, about 70% to about 80%, about 70% to about 75%, about 75% to about 100%, about 75% to about 95%, about 75% to about 90%, about 75% to about 85%, about 75% to about 80%, about 80% to about 100%, about 80% to about 95%, about 80% to about 90%, about 80% to about 85%, about 85% to about 100%, about 85% to about 95%, about 85% to about 90%, about 90% to about 100%, about 90% to about 95%, or about 95% to about 100% compared to prior to contacting the cell with the at least one epigenetic modifying agent.

104. The method of any one of claims 96-103, wherein the site-specific targeting moiety comprises a polymeric molecule.

105. The method of claim 104, wherein the polymeric molecule comprises a polyamide.

106. The method of claim 104 or 105, wherein the polymeric molecule comprises a polynucleotide.

107. The method of any one of claims 96-106, wherein the expression control region comprises a specific transcriptional control element for CXCL9, CXCL10, or CXCL11.

108. The method of any one of claims 96-107, wherein the epigenetic modifying agent targets at least two specific transcriptional control elements, each of which independently reduces transcription of CXCL9, CXCL10 or CXCL11.

109. The method of claim 107 or 108, wherein the transcriptional control element comprises a promoter for CXCL9, CXCL10, or CXCL11.

110. The method of claim 109, wherein the transcriptional control element comprises an enhancer for CXCL9, CXCL10, or CXCL11.

111. The method of any one of claims 96-110, wherein the epigenetic modifying agent comprises a nucleotide sequence having at least 85%, 90%, 95%, 96%, 97%, 98% or 99% nucleotide identity to the entire nucleotide sequence of any of the nucleotide sequences in any one of Table 12 or Table 13.

112. The method of any one of claims 96-111, wherein the epigenetic modifying agent comprises a polynucleotide encoding a Cas or dCas polypeptide, a DNA-binding domain of a Transcription activator-like effector (TALE) polypeptide, or a zinc finger (ZFP) polypeptide, or fragment thereof, that specifically targets the expression control region of CXCL9, CXCL10, or CXCL11.

113. The method of claim 112, wherein the DNA-binding domain of the ZFP or TALE comprises an amino acid sequence having at least 85%, 90%, 95%, 96%, 97%, 98% or 99% amino acid identity to the entire amino acid sequence of an amino acid sequence selected from the amino acid sequences listed in Table 3 or Table 5.

114. The method of any one of claims 96-113, wherein the expression control region comprises
1) one or more CXCL9-associated anchor sequences within an anchor sequence-mediated conjunction comprising a first and a second CXCL9-associated anchor sequence; and/or
2) one or more CXCL10-associated anchor sequences within an anchor sequence-mediated conjunction comprising a first and a second CXCL10-associated anchor sequence; and/or
3) one or more CXCL11-associated anchor sequences within an anchor sequence-mediated conjunction comprising a first and a second CXCL11-associated anchor sequence.

115. The method of claim 114, wherein the anchor sequence comprises a CCCTC-binding factor (CTCF) binding motif.

116. The method of claim 114 or 115, wherein the anchor sequence-mediated conjunction comprises one or more transcriptional control elements internal to the conjunction.

117. The method of claim 114 or 115, wherein the anchor sequence-mediated conjunction comprises one or more transcriptional control elements external to the conjunction.

118. The method of any one of claims 114-117, wherein the first and/or the second anchor sequence is located within about 500 kb of the transcriptional control element.

119. The method of claim 118, wherein the first and/or the second anchor sequence is located within about 300 kb of the transcriptional control element.

120. The method of any one of claims 114-119, wherein the anchor sequence is located within 10 kb of the transcriptional control element.

121. The method of any one of claims 96-120, wherein the expression control region comprises a specific transcriptional control element for CXCL9, CXCL10, or CXCL11.

122. The method of claim 121, wherein the transcriptional control element comprises a promoter for CXCL9, CXCL10, or CXCL11.

123. The method of claim 121, wherein the transcriptional control element comprises a transcriptional repressor.

124. The method of any one of claims 96-123, wherein the epigenetic modifying agent comprises a polynucleotide encoding a Cas or dCas polypeptide, a DNA-binding domain, or fragment thereof, of a zinc finger polypeptide (ZFP) that specifically binds to the expression control region of CXCL9, CXCL10, or CXCL11, or a transcription activator-like effector (TALE) polypeptide that specifically binds to the expression control region of CXCL9, CXCL10, or CXCL11.

125. The method of claim 124, wherein the DNA-binding domain of the ZFP comprises an amino acid sequence having at least 85%, 90%, 95%, 96%, 97%, 98% or 99% amino acid identity to the entire amino acid sequence of an amino acid sequence selected from the amino acid sequences listed in Table 3.

126. The method of any one of claims 96-125, wherein the epigenetic modifying agent comprises a nucleotide modification.

127. The method of any one of claims 104-126, wherein the polymeric molecule comprises a peptide nucleic acid (PNA).

128. The method of any one of claims 96-127, wherein the effector molecule comprises a polypeptide.

129. The method of claim 128, wherein the polypeptide comprises a nucleic acid molecule encoding a fusion protein comprising the site-specific targeting moiety which targets an expression regulatory region of CXCL9, CXCL10, or CXCL11, and the effector molecule.

130. The method of claim 129, wherein the fusion protein comprises a peptide-nucleic acid fusion molecule.

131. The method of any one of claims 96-130, wherein the effector is selected from the group consisting of a nuclease, a physical blocker, an epigenetic recruiter, and an epigenetic modifier, and combinations of any of the foregoing.

132. The method of claim 131, wherein the effector is selected from the group consisting of euchromatic histone-lysine N-methyltransferase 2 (G9a), enhancer of zeste homolog 2 (EZH2), MQ1 domain (MQ1), Krüppel associated box (KRAB) transcriptional repression domain, and histone deacetylase 8 (HDAC8).

133. The method of claim 132, wherein the effector is MQ1.

134. The method of claim 132, wherein the effector is KRAB.

135. The method of claim 131, wherein the effector comprises a CRISPR associated protein (Cas) polypeptide or nucleic acid molecule encoding the Cas polypeptide.

136. The method of claim 135, wherein the Cas polypeptide is an enzymatically inactive Cas polypeptide.

137. The method of claim 135 or 136, further comprising a catalytically active domain of human exonuclease 1 (hEXO1).

138. The method of claim 135, wherein the epigenetic modifier comprises a transcriptional repressor.

139. The method of claim 138, wherein the transcriptional repressor is selected from the group comprising euchromatic histone-lysine N-methyltransferase 2 (G9a), enhancer of zeste homolog 2 (EZH2), MQ1 domain (MQ1), Krüppel associated box (KRAB) transcriptional repression domain, and histone deacetylase 8 (HDAC8).

140. The method of claim 139, wherein the transcriptional repressor is MQ1 domain.

141. The method of claim 140, wherein the MQ1 domain comprises an amino acid sequence having at least about 85%, 90%, 95%, 96%, 97%, 98% or 99% amino acid identity to the entire amino acid sequence of SEQ ID NO: 79.

142. The method of any one of claims 139-141, wherein the transcriptional repressor comprises two, three, four, or five MQ1s.

143. The method of claim 139, wherein the transcriptional repressor is EZH2.

144. The method of claim 143, wherein the EZH2 has an amino acid sequence having at least about 85%, 90%, 95%, 96%, 97%, 98% or 99% amino acid identity to the entire amino acid sequence of SEQ ID NO: 78.

145. The method of claim 131, wherein the epigenetic modifier comprises a DNA methylase or a histone deacetylase.

146. The method of any one of claims 128-131, wherein the effector molecule comprises a Krüppel associated box (KRAB) transcriptional repression domain.

147. The method of claim 146, wherein the KRAB comprises an amino acid sequence having at least about 85%, 90%, 95%, 96%, 97%, 98% or 99% identity to the entire amino acid sequence of SEQ ID NO: 297.

148. The method of any one of claims 128-131, wherein the effector molecule comprises a Transcription activator-like effector nuclease (TALEN) polypeptide.

149. The method of claim 129, wherein the fusion protein comprises an enzymatically inactive Cas polypeptide and an epigenetic recruiter polypeptide.

150. The method of claim 129, wherein the fusion protein comprises an enzymatically active Cas polypeptide and an epigenetic modifier polypeptide.

151. The method of any one of claims 96-150, wherein the epigenetic modifying agent comprises a second nucleic acid molecule encoding a second fusion protein, wherein
1) the second fusion protein comprises a second site-specific targeting moiety which targets a second expression control region of CXCL9, CXCL10 or CXCL11, which is the same or different than the target of the first site-specific targeting moiety;
2) the second fusion comprises a second site-specific targeting moiety which targets a second CXCL9 expression control region and a second effector molecule, wherein the second CXCL9 expression control region is different than the CXCL9 expression control region; and/or
3) the second fusion comprises a second site-specific targeting moiety which targets a second CXCL10 expression control region and a second effector molecule, wherein the second CXCL10 expression control region is different than the CXCL10 expression control region; and/or
4) the second fusion comprises a second site-specific targeting moiety which targets a second CXCL11 expression control region and a second effector molecule, wherein the second CXCL10 expression control region is different than the CXCL11 expression control region.

152. The method of any one of claims 96-151, wherein
1) the epigenetic modifying agent targets a CXCL9 expression control region comprising either a ZF target sequence comprising SEQ ID NOs: 422-425, or TALE target sequence comprising SEQ ID NOs: 429-432, or a combination thereof and, optionally, a second CXCL9 expression control region comprising either a ZF target sequence comprising SEQ ID NOs: 422-425, or TALE target sequence comprising SEQ ID NOs: 429-432, or a combination thereof; and/or
2) the epigenetic modifying agent targets a CXCL10 expression control region comprising either a ZF target sequence comprising SEQ ID NOs: 422-425, or TALE target sequence comprising SEQ ID NOs: 429-432, or a combination thereof and, optionally, a second CXCL10 expression control region comprising either a ZF target sequence comprising SEQ ID NOs: 422-425, or TALE target sequence comprising SEQ ID NOs: 429-432, or a combination thereof; and/or
3) the epigenetic modifying agent targets a CXCL11 expression control region comprising either a ZF target sequence comprising SEQ ID NOs: 422-425, or TALE target sequence comprising SEQ ID NOs: 429-432, or a combination thereof and, optionally, a second CXCL11 expression control region comprising either a ZF target sequence comprising SEQ ID NOs: 422-425, or TALE target sequence comprising SEQ ID NOs: 429-432, or a combination thereof.

153. The method of any one of claims 96-152, wherein
1) the epigenetic modifying agent targets a CXCL9 expression control region comprising either a ZF target sequence comprising SEQ ID NOs: 422-425, or TALE target sequence comprising SEQ ID NOs: 429-432, or a combination thereof, and a second CXCL9 expression control region comprising either a ZF target sequence comprising SEQ ID NOs: 422-425, or TALE target sequence comprising SEQ ID NOs: 429-432, or a combination thereof; and/or
2) the epigenetic modifying agent targets a CXCL10 expression control region comprising either a ZF target sequence comprising SEQ ID NOs: 422-425, or TALE target sequence comprising SEQ ID NOs: 429-432, or a combination thereof, and a second CXCL10 expression control region comprising either a ZF target sequence comprising SEQ ID NOs: 422-425, or TALE target sequence comprising SEQ ID NOs: 429-432, or a combination thereof; and/or
3) the epigenetic modifying agent targets a CXCL11 expression control region comprising either a ZF target sequence comprising SEQ ID NOs: 422-425, or TALE target sequence comprising SEQ ID NOs: 429-432, or a combination thereof, and a second CXCL11 expression control region comprising either a ZF target sequence comprising SEQ ID NOs: 422-425, or TALE target sequence comprising SEQ ID NOs: 429-432, or a combination thereof.

154. The method of any one of claims 151-153, wherein the second effector is different than the effector.

155. The method of any one of claims 151-153, wherein the second effector is the same as the effector.

156. The method of any one of claims 151-155, wherein the fusion protein and the second fusion protein are operably linked.

157. The method of claim 156, wherein the fusion protein and the second fusion protein independently comprise an amino acid sequence that has at least about 85%, 90%, 95%, 96%, 97%, 98% or 99% sequence identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

158. The method of any one of claims 96-157, wherein the fusion protein comprises an amino acid sequence having at least 85%, 90%, 95%, 96%, 97%, 98% or 99% sequence identity to the entire amino acid sequence of a polypeptide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

159. The method of claim 158, wherein the polypeptide is ZF05-KRAB.

160. The method of claim 158, wherein the polypeptide is ZF07-KRAB.

161. The method of claim 158, wherein the polypeptide is ZF16-KRAB.

162. The method of claim 158, wherein the polypeptide is ZF24-KRAB.

163. The method of claim 158, wherein the polypeptide is ZF05-MQ1.

164. The method of claim 158, wherein the polypeptide is ZF07-MQ1.

165. The method of claim 158, wherein the polypeptide is TAL04-KRAB.

166. The method of claim 158, wherein the polypeptide is TAL08-KRAB.

167. The method of claim 158, wherein the polypeptide is TAL09-KRAB.

168. The method of claim 158, wherein the polypeptide is TAL14-KRAB.

169. The method of any one of claims 158-168, wherein the fusion protein, and optionally, the second fusion protein, are independently encoded by a polynucleotide comprising a nucleotide sequence that has at least about 85%, 90%, 95%, 96%, 97%, 98% or 99% nucleotide sequence identity to the entire nucleotide sequence of a polynucleotide selected from the group consisting of ZF05-KRAB (SEQ ID NO: 467), ZF07-KRAB (SEQ ID NO: 461), ZF16-KRAB (SEQ ID NO: 464), ZF24-KRAB (SEQ ID NO: 463), ZF05-MQ1 (SEQ ID NO: 466), ZF07-MQ1 (SEQ ID NO: 465), TAL04-KRAB (SEQ ID NO: 471), TAL08-KRAB (SEQ ID NO: 470), TAL09-KRAB (SEQ ID NO: 469), and TAL14-KRAB (SEQ ID NO: 468).

170. The method of any one of claims 152-169, wherein the administration of the epigenetic modifying agent and the second epigenetic modifying agent has a synergistic effect in reducing the expression of CXCL9, CXCL10, or CXCL11.

171. The method of any one of claims 100-153, wherein the expression control region comprises a sequence selected from ZF24 target sequence (SEQ ID NO: 422), ZF16 target sequence (SEQ ID NO: 423), ZF07 target sequence (SEQ ID NO: 424), ZF05 target sequence (SEQ ID NO: 425), TAL04 target sequence (SEQ ID NO: 429), TAL08 target sequence (SEQ ID NO: 430), TAL09 target sequence (SEQ ID NO: 431), and TAL14 target sequence (SEQ ID NO: 432).

172. The method of claim 171, wherein the first expression control region comprises a ZF05 target sequence (SEQ ID NO: 425) and the second expression control region comprises a TAL08 target sequence (SEQ ID NO: 430) or TAL14 target sequence (SEQ ID NO: 432).

173. The method of claim 171, wherein the expression control region comprises a ZF05 target sequence (SEQ ID NO: 425).

174. The method of any one of claims 96-173, wherein the epigenetic modifying agent, the effector, or both the epigenetic modifying agent and the effector are present in a vector.

175. The method of claim 174, wherein the epigenetic modifying agent and the effector are present in the same vector.

176. The method of claim 174, wherein the epigenetic modifying agent and the effector are present in different vectors.

177. The method of any one of claims 174-176, wherein the vector is a viral expression vector.

178. The method of any one of claims 96-173, wherein the epigenetic modifying agent, the effector, or both the epigenetic modifying agent and the effector are present in a composition.

179. The method of claim 178, wherein the epigenetic modifying agent and the effector are present in the same composition.

180. The method of claim 178, wherein the epigenetic modifying agent and the effector are present in different compositions.

181. The method of any one of claims 178-180, wherein the composition comprises a pharmaceutical composition.

182. The method of claim 181, wherein the pharmaceutical composition comprises a lipid formulation.

183. The method of claim 182, wherein the lipid formulation comprises one or more cationic lipids, one or more non-cationic lipids, one or more cholesterol-based lipids, or one or more PEG-modified lipids, or combinations of any of the foregoing.

184. The method of claim 183, wherein the pharmaceutical composition comprises a lipid nanoparticle.

185. The method of any one of claims 96-184, wherein the cell is a mammalian cell.

186. The method of claim 185, wherein the mammalian cell is a somatic cell.

187. The method of claim 185, wherein the mammalian cell is a primary cell.

188. The method of any one of claims 96-185, wherein the cell is an immune cell.

189. The method of any one of claims 96-188, wherein the contacting is performed in vitro.

190. The method of any one of claims 96-188, wherein the contacting is performed in vivo.

191. The method of any one of claims 96-188, wherein the contacting is performed ex vivo.

192. The method of claim 191, further comprising administering the cell to a subject.

193. The method of any one of claims 96-192, wherein the cell is within a subject.

194. The method of claim 192 or 193, wherein the subject has a disease associated with CXCL9, CXCL10, and/or CXCL11.

195. The method of claim 194, wherein the disease associated with CXCL9, CXCL10, and/or CXCL11 is selected from the group consisting of acute and chronic liver disease, alagille syndrome, autoimmune hepatitis, biliary atresia, cirrhosis, endoscopic retrograde cholangiopancreatography (ERCP), hemochromatosis, hepatitis A, hepatitis B, alopecia areata, multiple sclerosis, chronic pain, and Human T-lymphotropic virus type 1 (HTLV-1)-associated myelopathy/tropical spastic paraparesis (HAM/TSP).

196. A method for treating a subject having a disease associated with CXCL9, CXCL10, and/or CXCL11, comprising administering to the subject a therapeutically effective amount of at least one epigenetic modifying agent, each epigenetic modifying agent comprising at least one site-specific targeting moiety which targets a target sequence in an expression control region of human CXCL9, CXCL10, or CXCL11, and an effector molecule, thereby treating the subject.

197. A method for decreasing T cell migration in a subject having a disease, comprising administering to the subject a therapeutically effective amount of at least one epigenetic modifying agent, each epigenetic modifying agent comprising at least one site-specific targeting moiety which targets a target sequence in an expression control region of human CXCL9, CXCL10, or CXCL11, and an effector molecule, thereby decreasing T cell migration the subject.

198. The method of claim 197, wherein T cell migration is decreased in the subject is reduced by at least 10%, at least 15%, at least 20%, at least 25%, at least 30%, at least 35%, at least 40%, at least 45%, at least 50%, at least 55%, at least 60%, at least 65%, or at least 70% compared to prior to administering the at least one epigenetic modifying agent to the subject.

199. The method of claim 197 or 198, wherein T cell migration is decreased in the subject is reduced by between about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 70%, about 30% to about 65%, about 30% to about 60%, about 30% to about 55%, about 30% to about 50%, about 30% to about 45%, about 30% to about 40%, about 30% to about 35%, about 35% to about 70%, about 35% to about 65%, about 35% to about 60%, about 35% to about 55%, about 35% to about 50%, about 35% to about 45%, about 35% to about 40%, about 40% to about 70%, about 40% to about 65%, about 40% to about 60%, about 40% to about 55%, about 40% to about 50%, about 40% to about 45%, about 45% to about 70%, about 45% to about 65%, about 45% to about 60%, about 45% to about 55%, about 45% to about 50%, about 50% to about 70%, about 50% to about 65%, about 50% to about 60%, about 50% to about 55%, about 55% to about 70%, about 55% to about 65%, about 55% to about 60%, about 60% to about 70%, about 60% to about 65%, or about 65% to about 70% compared to prior to administering the at least one epigenetic modifying agent to the subject.

200. The epigenetic modifying agent of any one of claims 196-199, wherein the target sequence is within a human chromosomal region selected from the group consisting of chr4: 76928543-76931536, chr4: 76944524-76948668, chr4: 76957098-76962383, chr4: 76949523-76949873, chr4: 76932906-76933929, chr4: 76916900-76917300, chr4: 76988761-76989260, chr4: 77008274-77008727, and chr4: 76957852-76958551.

201. The epigenetic modifying agent of any one of claims 196-199, wherein the target sequence is located within about 100 bp, about 200 bp, about 300 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, or about 3.0 kb from a human chromosomal region selected from the group consisting of chr4: 76958201-76958221, chr4: 76949758-76949778, chr4: 76933245-76933265, chr4: 76932967-76932987, chr4: 76957115-76957132, chr4: 76944734-76944751, chr4: 76944656-76944673, and chr4: 76933040-76933057.

202. The epigenetic modifying agent of any one of claims 196-200, wherein the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

203. The method of any one of claims 196-202, wherein the disease associated with CXCL9, CXCL10, and/or CXCL11 is selected from the group consisting of acute and chronic liver disease, alagille syndrome, autoimmune hepatitis, biliary atresia, cirrhosis, endoscopic retrograde cholangiopancreatography (ERCP), hemochromatosis, hepatitis A, hepatitis B, alopecia areata, multiple sclerosis, chronic pain, and Human T-lymphotropic virus type 1 (HTLV-1)-associated myelopathy/tropical spastic paraparesis (HAM/TSP) and the epigenetic modifying agent reduces expression of CXCL9, CXCL10, or CXCL11 in the subject.

204. The method of any one of claims 196-203, wherein the epigenetic modifying agent and the effector molecule are administered to the subject concurrently.

205. The method of any one of claims 196-203, wherein the epigenetic modifying agent and the effector molecule are administered to the subject sequentially.

206. The method of claim 205, wherein the effector molecule is administered to the subject prior to administration of the epigenetic modifying agent.

207. The method of claim 205, wherein the epigenetic modifying agent is administered to the subject prior to administration of the effector molecule.

208. An epigenetic modifying agent, comprising at least one site-specific targeting moiety which targets a target sequence within an expression control region of human CXCL9, CXCL10, or CXCL11, wherein the target sequence is within a human chromosomal region selected from the group consisting of chr4: 76928543-76931536, chr4: 76944524-76948668, chr4: 76957098-76962383, chr4: 76949523-76949873, chr4: 76932906-76933929, chr4: 76916900-76917300, chr4: 76988761-76989260, chr4: 77008274-77008727, and chr4: 76957852-76958551.

209. The epigenetic modifying agent of claim 208, wherein the target sequence comprises a sequence selected from the group consisting of AGCCATGACACTGCACTACCC (SEQ ID NO: 422), ATTCTGCACCAGCTGAGGGGA (SEQ ID NO: 423), GATCCAAATGCTAATGTAACC (SEQ ID NO: 424), GCATGACAAGTTCCTAAACGA (SEQ ID NO: 425), GAAGGGCATGGCTATAGC (SEQ ID NO: 429), GACTTAGCAAAACCTGCT (SEQ ID NO: 430), GGCACACTAGCCCCACGT (SEQ ID NO: 431), and GCAACGATCAATGGGATT (SEQ ID NO: 432).

210. The epigenetic modifying agent of claim 208 or 209, wherein the site-specific targeting moiety comprises a nucleotide sequence having at least 85%, 90%, 95%, 96%, 97%, 98% or 99% nucleotide identity to the entire nucleotide sequence of any of the nucleotide sequences in any one of Table 3 or Table 5.

211. The epigenetic modifying agent of any one of claims 208-210, wherein the site-specific targeting moiety comprises a polymeric molecule comprising a polynucleotide encoding a DNA-binding domain of a Transcription activator-like effector (TALE) polypeptide or a zinc finger (ZFP) polypeptide, or fragment thereof, that specifically binds to the expression control region of CXCL9, CXCL10, or CXCL11.

212. The epigenetic modifying agent of claim 210, wherein the DNA-binding domain of the ZFP or TALE polypeptide comprises an amino acid sequence having at least about 85%, 90%, 95%, 96%, 97%, 98% or 99% amino acid identity to the entire amino acid sequence of any one of the amino acid sequences listed in Table 3 or Table 5.

213. The epigenetic modifying agent of claim 208, wherein the expression control region comprises a nucleotide sequence of a ZF05 target sequence (SEQ ID NO: 407).

214. The epigenetic modifying agent of claim 208, wherein the expression control region comprises a nucleotide sequence of a ZF07 target sequence (SEQ ID NO: 405).

215. The epigenetic modifying agent of claim 208, wherein the expression control region comprises a nucleotide sequence of a ZF16 target sequence (SEQ ID NO: 403).

216. The epigenetic modifying agent of claim 208, wherein the expression control region comprises a nucleotide sequence of a ZF24 target sequence (SEQ ID NO: 401).

217. The epigenetic modifying agent of claim 208, wherein the expression control region comprises a nucleotide sequence of a TAL08 target sequence (SEQ ID NO: 419).

218. The epigenetic modifying agent of claim 208, wherein the expression control region comprises a nucleotide sequence of a TAL14 target sequence (SEQ ID NO: 415).

219. The epigenetic modifying agent of claim 208, wherein the expression control region comprises a nucleotide sequence of a TAL04 target sequence (SEQ ID NO: 421).

220. The epigenetic modifying agent of claim 208, wherein the expression control region comprises a nucleotide sequence of a TAL09 target sequence (SEQ ID NO: 417).

221. An epigenetic modifying agent, comprising a polynucleotide encoding the amino acid sequence of ZF05-MQ1 comprising the amino acid sequence of (SEQ ID NO: 466).

222. An epigenetic modifying agent, comprising a polynucleotide encoding the amino acid sequence of ZF07-MQ1 comprising the amino acid sequence of (SEQ ID NO: 465).

223. An epigenetic modifying agent, comprising a polynucleotide encoding the amino acid sequence of ZF05-KRAB comprising the amino acid sequence of (SEQ ID NO: 467).

224. An epigenetic modifying agent, comprising a polynucleotide encoding the amino acid sequence of ZF07-KRAB comprising the amino acid sequence of (SEQ ID NO: 461).

225. An epigenetic modifying agent, comprising a polynucleotide encoding the amino acid sequence of ZF24-KRAB comprising the amino acid sequence of (SEQ ID NO: 463).

226. An epigenetic modifying agent, comprising a polynucleotide encoding the amino acid sequence of ZF16-KRAB comprising the amino acid sequence of (SEQ ID NO: 464).

227. An epigenetic modifying agent, comprising a polynucleotide encoding the amino acid sequence of TAL08-KRAB comprising the amino acid sequence of (SEQ ID NO: 470).

228. An epigenetic modifying agent, comprising a polynucleotide encoding the amino acid sequence of a TAL14-KRAB comprising the amino acid sequence of (SEQ ID NO: 468).

229. An epigenetic modifying agent, comprising a polynucleotide encoding the amino acid sequence of a TAL04-KRAB comprising the amino acid sequence of (SEQ ID NO: 471).

230. An epigenetic modifying agent, comprising a polynucleotide encoding the amino acid sequence of a TAL09-KRAB comprising the amino acid sequence of (SEQ ID NO: 469).

231. An epigenetic modifying agent, comprising a polynucleotide encoding the amino acid sequence of a ZF05-KRAB-tPT2A-TAL14-KRAB comprising the amino acid sequence of (SEQ ID NO: 474).

232. A vector comprising a nucleic acid molecule encoding the epigenetic modifying agent of any one of claims 221-231.

233. The vector of claim 232, wherein the vector is a viral expression vector.

234. A pharmaceutical composition comprising the epigenetic modifying agent of any one of claims 221-231, or the vector of claim 232 or 233.

235. The pharmaceutical composition of claim 234, wherein the pharmaceutical composition comprises a lipid formulation.

236. The pharmaceutic composition of claim 235, wherein the lipid formulation comprises one or more cationic lipids, one or more non-cationic lipids, one or more cholesterol-based lipids, or one or more PEG-modified lipids, or combinations of any of the foregoing.

237. The pharmaceutical composition of claim 235, wherein the pharmaceutical composition comprises a lipid nanoparticle.

238. A pharmaceutical composition, comprising an epigenetic modifying agent, comprising a polynucleotide encoding the amino acid sequence of ZF05-MQ1 comprising the amino acid sequence of (SEQ ID NO: 466); and a lipid nanoparticle.

239. A pharmaceutical composition, comprising an epigenetic modifying agent, comprising a polynucleotide encoding the amino acid sequence of ZF07-MQ1 comprising the amino acid sequence of (SEQ ID NO: 465); and a lipid nanoparticle.

240. A pharmaceutical composition, comprising an epigenetic modifying agent, comprising a polynucleotide encoding the amino acid sequence of a ZF05-KRAB comprising the amino acid sequence of (SEQ ID NO: 467); and a lipid nanoparticle.

241. A pharmaceutical composition, comprising an epigenetic modifying agent, comprising a polynucleotide encoding the amino acid sequence of a ZF07-KRAB comprising the amino acid sequence of (SEQ ID NO: 461); and a lipid nanoparticle.

242. A pharmaceutical composition, comprising an epigenetic modifying agent, comprising a polynucleotide encoding the amino acid sequence of a ZF24-KRAB comprising the amino acid sequence of (SEQ ID NO: 463); and a lipid nanoparticle.

243. A pharmaceutical composition, comprising an epigenetic modifying agent, comprising a polynucleotide encoding the amino acid sequence of a ZF16-KRAB comprising the amino acid sequence of (SEQ ID NO: 464); and a lipid nanoparticle.

244. A pharmaceutical composition, comprising an epigenetic modifying agent, comprising a polynucleotide encoding the amino acid sequence of a TAL08-KRAB comprising the amino acid sequence of (SEQ ID NO: 470); and a lipid nanoparticle.

245. A pharmaceutical composition, comprising an epigenetic modifying agent, comprising a polynucleotide encoding the amino acid sequence of a TAL14-KRAB comprising the amino acid sequence of (SEQ ID NO: 468); and a lipid nanoparticle.

246. A pharmaceutical composition, comprising an epigenetic modifying agent, comprising a polynucleotide encoding the amino acid sequence of a TAL04-KRAB comprising the amino acid sequence of (SEQ ID NO: 471); and a lipid nanoparticle.

247. A pharmaceutical composition, comprising an epigenetic modifying agent, comprising a polynucleotide encoding the amino acid sequence of a TAL09-KRAB comprising the amino acid sequence of (SEQ ID NO: 469); and a lipid nanoparticle.

248. A pharmaceutical composition, comprising an epigenetic modifying agent, comprising a polynucleotide encoding the amino acid sequence of a ZF05-KRAB-tPT2A-TAL14-KRAB comprising the amino acid sequence of (SEQ ID NO: 474); and a lipid nanoparticle.

249. A cell comprising the epigenetic modifying agent of any one of claims 221-231, or the vector of claim 232 or 233.

250. A method of reducing expression of CXCL9, CXCL10, or CXCL11 in a cell, the method comprising contacting the cell with an epigenetic modifying agent of any one of claims 221-231, or the vector of claim 232 or 233, or the pharmaceutical composition of any one claims 234-248.

251. The method of claim 250, wherein the expression of CXCL9, CXCL10, or CXCL11 in the cell is reduced by at least 30%, at least 35%, at least 40%, at least 45%, at least 50%, at least 55%, at least 60%, at least 65%, at least 70%, at least 75%, at least 80%, at least 85%, at least 90%, or at least 95% compared to prior to contacting the cell with the at least one epigenetic modifying agent.

252. The method of claim 250 or 251, wherein the expression of CXCL9, CXCL10, or CXCL11 in the cell is reduced between about 30% to about 100%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 35% to about 100%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 40% to about 100%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 100%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 100%, about 55% to about 95%, about 55% to about 90%, about 55% to about 85%, about 55% to about 80%, about 55% to about 75%, about 55% to about 70%, about 55% to about 65%, about 55% to about 60%, about 60% to about 100%, about 60% to about 95%, about 60% to about 90%, about 60% to about 85%, about 60% to about 80%, about 60% to about 75%, about 60% to about 70%, about 60% to about 65%, about 65% to about 100%, about 65% to about 95%, about 65% to about 90%, about 65% to about 85%, about 65% to about 80%, about 65% to about 75%, about 65% to about 70%, about 70% to about 100%, about 70% to about 95%, about 70% to about 90%, about 70% to about 85%, about 70% to about 80%, about 70% to about 75%, about 75% to about 100%, about 75% to about 95%, about 75% to about 90%, about 75% to about 85%, about 75% to about 80%, about 80% to about 100%, about 80% to about 95%, about 80% to about 90%, about 80% to about 85%, about 85% to about 100%, about 85% to about 95%, about 85% to about 90%, about 90% to about 100%, about 90% to about 95%, or about 95% to about 100% compared to prior to contacting the cell with the at least one epigenetic modifying agent.


SEQUENCE LISTING (474 sequences)

SEQ ID NO: 1 - protein, 7 aa, synthetic construct
  REGION 1..7: note = Synthetic
RKDALRG

SEQ ID NO: 2 - protein, 7 aa, synthetic construct
  REGION 1..7: note = Synthetic
TTGALTE

SEQ ID NO: 3 - protein, 7 aa, synthetic construct
  REGION 1..7: note = Synthetic
QRHHLVE

SEQ ID NO: 4 - protein, 7 aa, synthetic construct
  REGION 1..7: note = Synthetic
QNSTLTE

SEQ ID NO: 5 - protein, 7 aa, synthetic construct
  REGION 1..7: note = Synthetic
RNDALTE

SEQ ID NO: 6 - protein, 7 aa, synthetic construct
  REGION 1..7: note = Synthetic
HKNALQN

SEQ ID NO: 7 - protein, 7 aa, synthetic construct
  REGION 1..7: note = Synthetic
RRSACRR

SEQ ID NO: 8 - protein, 7 aa, synthetic construct
  REGION 1..7: note = Synthetic
QKSSLIA

SEQ ID NO: 9 - protein, 7 aa, synthetic construct
  REGION 1..7: note = Synthetic
RRDELNV

SEQ ID NO: 10 - protein, 7 aa, synthetic construct
  REGION 1..7: note = Synthetic
TSGSLVR

SEQ ID NO: 11 - protein, 7 aa, synthetic construct
  REGION 1..7: note = Synthetic
DPGALVR

SEQ ID NO: 12 - protein, 7 aa, synthetic construct
  REGION 1..7: note = Synthetic
QSSSLVR

SEQ ID NO: 13 - protein, 7 aa, synthetic construct
  REGION 1..7: note = Synthetic
RSDELVR

SEQ ID NO: 14 - protein, 7 aa, synthetic construct
  REGION 1..7: note = Synthetic
RLRDIQF

SEQ ID NO: 15 - protein, 7 aa, synthetic construct
  REGION 1..7: note = Synthetic
RSDERKR

SEQ ID NO: 16 - protein, 7 aa, synthetic construct
  REGION 1..7: note = Synthetic
RSDHLTT

SEQ ID NO: 17 - protein, 7 aa, synthetic construct
  REGION 1..7: note = Synthetic
RLRALDR

SEQ ID NO: 18 - protein, 7 aa, synthetic construct
  REGION 1..7: note = Synthetic
TKNSLTE

SEQ ID NO: 19 - protein, 7 aa, synthetic construct
  REGION 1..7: note = Synthetic
SKKHLAE

SEQ ID NO: 20 - protein, 7 aa, synthetic construct
  REGION 1..7: note = Synthetic
TSHSLTE

SEQ ID NO: 21 - protein, 7 aa, synthetic construct
  REGION 1..7: note = Synthetic
RNDTLTE

SEQ ID NO: 22 - protein, 7 aa, synthetic construct
  REGION 1..7: note = Synthetic
THLDLIR

SEQ ID NO: 23 - protein, 7 aa, synthetic construct
  REGION 1..7: note = Synthetic
DKKDLTR

SEQ ID NO: 24 - protein, 7 aa, synthetic construct
  REGION 1..7: note = Synthetic
SPADLTR

SEQ ID NO: 25 - protein, 7 aa, synthetic construct
  REGION 1..7: note = Synthetic
RTDTLRD

SEQ ID NO: 26 - protein, 7 aa, synthetic construct
  REGION 1..7: note = Synthetic
TSGELVR

SEQ ID NO: 27 - protein, 7 aa, synthetic construct
  REGION 1..7: note = Synthetic
DCRDLAR

SEQ ID NO: 28 - protein, 7 aa, synthetic construct
  REGION 1..7: note = Synthetic
QSGDLRR

SEQ ID NO: 29 - protein, 7 aa, synthetic construct
  REGION 1..7: note = Synthetic
RSDDLVR

SEQ ID NO: 30 - protein, 7 aa, synthetic construct
  REGION 1..7: note = Synthetic
ARGNLRT

SEQ ID NO: 31 - protein, 7 aa, synthetic construct
  REGION 1..7: note = Synthetic
SRGNLKS

SEQ ID NO: 32 - protein, 7 aa, synthetic construct
  REGION 1..7: note = Synthetic
QASNLIS

SEQ ID NO: 33 - protein, 7 aa, synthetic construct
  REGION 1..7: note = Synthetic
REDNLHT

SEQ ID NO: 34 - protein, 7 aa, synthetic construct
  REGION 1..7: note = Synthetic
TSGNLTE

SEQ ID NO: 35 - protein, 7 aa, synthetic construct
  REGION 1..7: note = Synthetic
SKKALTE

SEQ ID NO: 36 - protein, 7 aa, synthetic construct
  REGION 1..7: note = Synthetic
QSGNLTE

SEQ ID NO: 37 - protein, 7 aa, synthetic construct
  REGION 1..7: note = Synthetic
RADNLTE

SEQ ID NO: 38 - protein, 7 aa, synthetic construct
  REGION 1..7: note = Synthetic
TTGNLTV

SEQ ID NO: 39 - protein, 7 aa, synthetic construct
  REGION 1..7: note = Synthetic
DSGNLRV

SEQ ID NO: 40 - protein, 7 aa, synthetic construct
  REGION 1..7: note = Synthetic
QRANLRA

SEQ ID NO: 41 - protein, 7 aa, synthetic construct
  REGION 1..7: note = Synthetic
RKDNLKN

SEQ ID NO: 42 - protein, 7 aa, synthetic construct
  REGION 1..7: note = Synthetic
TSGNLVR

SEQ ID NO: 43 - protein, 7 aa, synthetic construct
  REGION 1..7: note = Synthetic
DPGNLVR

SEQ ID NO: 44 - protein, 7 aa, synthetic construct
  REGION 1..7: note = Synthetic
QSSNLVR

SEQ ID NO: 45 - protein, 7 aa, synthetic construct
  REGION 1..7: note = Synthetic
RSDNLVR

SEQ ID NO: 46 - protein, 7 aa, synthetic construct
  REGION 1..7: note = Synthetic
APKALGW

SEQ ID NO: 47 - protein, 7 aa, synthetic construct
  REGION 1..7: note = Synthetic
QAGHLAS

SEQ ID NO: 48 - protein, 7 aa, synthetic construct
  REGION 1..7: note = Synthetic
RSDHLTT

SEQ ID NO: 49 - protein, 7 aa, synthetic construct
  REGION 1..7: note = Synthetic
SRRTCRA

SEQ ID NO: 50 - protein, 7 aa, synthetic construct
  REGION 1..7: note = Synthetic
HTGHLLE

SEQ ID NO: 51 - protein, 7 aa, synthetic construct
  REGION 1..7: note = Synthetic
QSGHLTE

SEQ ID NO: 52 - protein, 7 aa, synthetic construct
  REGION 1..7: note = Synthetic
RSDKLTE

SEQ ID NO: 53 - protein, 7 aa, synthetic construct
  REGION 1..7: note = Synthetic
HRTTLTN

SEQ ID NO: 54 - protein, 7 aa, synthetic construct
  REGION 1..7: note = Synthetic
ERSHLRE

SEQ ID NO: 55 - protein, 7 aa, synthetic construct
  REGION 1..7: note = Synthetic
QLAHLRA

SEQ ID NO: 56 - protein, 7 aa, synthetic construct
  REGION 1..7: note = Synthetic
RSDHLTN

SEQ ID NO: 57 - protein, 7 aa, synthetic construct
  REGION 1..7: note = Synthetic
TSGHLVR

SEQ ID NO: 58 - protein, 7 aa, synthetic construct
  REGION 1..7: note = Synthetic
DPGHLVR

SEQ ID NO: 59 - protein, 7 aa, synthetic construct
  REGION 1..7: note = Synthetic
QRAHLER

SEQ ID NO: 60 - protein, 7 aa, synthetic construct
  REGION 1..7: note = Synthetic
RSDKLVR

SEQ ID NO: 61 - protein, 11 aa, synthetic construct
  REGION 1..11: note = Synthetic
YKCPECGKSFS

SEQ ID NO: 62 - protein, 5 aa, synthetic construct
  REGION 1..5: note = Synthetic
HQRTH

SEQ ID NO: 63 - protein, 5 aa, synthetic construct
  REGION 1..5: note = Synthetic
TGEKP

SEQ ID NO: 64-71 - skipped (8 entries)

SEQ ID NO: 72 - DNA, 1128 bp, Homo sapiens
  misc_feature 1..1128: note = HDAC8 (NP_060956.1), Effector DNA sequence
  source 1..1128: mol_type = genomic DNA
gaagaacctgaggagcctgccgattccggacagagcctggtgcccgtgtatatctactcccccgaatacgtgtctatgtgcgacagcttggctaagattccgaagcgggcatcaatggtgcactcgctgatcgaggcctacgccctgcacaagcagatgagaatcgtgaagcccaaggtcgcctcaatggaagagatggccaccttccacaccgacgcctacttgcaacatctccaaaaagtgtcccaggaaggggacgacgaccaccctgactccattgagtacggcctgggttacgactgcccagctaccgaagggatcttcgactacgccgctgccattggcggagcgaccatcactgccgcgcagtgcctcatcgacggaatgtgcaaggtcgccatcaactggtccggaggctggcaccacgccaagaaggatgaagcctccggcttctgctaccttaacgacgccgtgctgggaatccttcgcctgcgaagaaagttcgaacggattttgtatgtggacctggatctgcatcatggagatggcgtggaggatgccttctccttcacctcgaaagtcatgaccgtgtcacttcacaagttctcacccggcttcttccctgggaccggagatgtgtccgacgtcggtctgggaaagggccgctactactccgtgaacgtgccgatccaggacggtatccaggacgagaagtactaccagatctgcgagagcgtgctgaaggaagtgtaccaggccttcaatcccaaggccgtggtgctgcagcttggcgcggacactattgcgggcgacccgatgtgctccttcaacatgacccccgtcggtattggaaagtgcctgaagtacatcctgcaatggcagctggctaccctgatcctgggaggaggcggatacaacctcgcaaacaccgcccggtgttggacttacctgactggggtcatcctgggaaagactctgtcatccgagattcccgaccacgagttcttcaccgcgtacggcccagattacgtgctcgaaatcactccttcctgccggcctgatagaaatgagccgcaccgcatacagcagatcctcaactacattaagggcaacctgaagcacgtggtg

SEQ ID NO: 73 - DNA, 1146 bp, Homo sapiens
  misc_feature 1..1146: note = G9A (NP_006700.3), Effector DNA sequence
  source 1..1146: mol_type = genomic DNA
ggaagcgccgctatcgccgaagtgctgctgaatgccagatgcgatctgcatgccgtgaactaccacggcgacacccctctgcatatcgccgctagagagagctaccatgactgtgtgctgctgtttctgagcagaggcgccaatcccgagctcagaaacaaagagggcgacaccgcttgggatctgacacccgagagatccgacgtgtggttcgctctgcaactgaatagaaaactgagactgggcgtcggcaatagggccattagaaccgagaagatcatctgtagggacgtggctaggggctacgagaacgtgcccatcccttgtgtgaacggagtggatggagagccttgccccgaggattacaaatacatcagcgagaactgcgaaacctccaccatgaatatcgatagaaacattacacacctccagcactgtacatgcgtggacgattgcagcagcagcaactgtctgtgcggccaactgagcatcagatgctggtacgacaaggatggcagactgctgcaagagttcaacaagatcgaaccccctctgatcttcgagtgtaaccaagcttgcagctgttggaggaactgcaagaatagggtcgtgcagtccggaatcaaggtgagactgcagctgtatagaacagctaagatgggatggggagtcagagctctgcagaccatcccccaaggcacattcatctgtgagtacgtcggcgaactcatcagcgacgctgaggccgatgtgagggaggacgacagctatctcttcgacctcgacaacaaggacggcgaggtgtactgcatcgacgctagatattacggcaacatcagcagattcatcaaccacctctgcgaccccaatatcatccccgtgagagtgttcatgctccatcaagatctgagattccctaggatcgccttcttcagctctagagacattagaaccggcgaggagctgggattcgactacggcgacaggttctgggacatcaagagcaagtacttcacatgccaatgcggcagcgagaaatgcaagcatagcgccgaggccattgctctggagcagtctagactggctaggctggaccctcaccccgagctgctgcccgaactgggatctctgcctcccgtgaatacc

SEQ ID NO: 74 - DNA, 2235 bp, Homo sapiens
  misc_feature 1..2235: note = EZH2 (NP_001190176.1), Effector DNA sequence
  source 1..2235: mol_type = genomic DNA
ggacagacgggcaaaaagagcgaaaagggacccgtctgctggcggaaacgagtgaaaagcgaatacatgcgcctgcggcaactcaagaggttccgcagagccgacgaggtcaagagcatgttcagcagcaacagacaaaagatcctggagaggaccgaaatcctcaaccaggaatggaagcagcgcaggatccagccggtgcacatcctgaccagcgtcagcagcctcagaggcacccgcgagtgcagcgtgaccagcgacctggacttccccacccaagtgatcccgctgaaaaccctcaacgcagtggccagcgtgccgatcatgtacagctggagccccctgcagcagaacttcatggtcgaggacgaaaccgtgctgcacaacatcccgtacatgggcgacgaagtgctggaccaggacggaaccttcatcgaggaactgatcaagaactacgacggaaaggtgcacggggacagggaatgcgggttcatcaacgacgagatcttcgtcgagctcgtcaacgccctgggacaatacaacgacgacgacgacgacgacgacggggacgacccagaagaacgggaggaaaagcagaaggacctggaggaccaccgcgacgacaaggaaagcagaccccccaggaagttcccgagcgacaagatcttcgaagccatcagcagcatgttcccggacaagggaaccgccgaggagctgaaggaaaagtacaaggaactgaccgagcagcagctcccaggagcactgccgccggaatgcacaccaaacatcgacggaccgaacgccaagagcgtgcagagggagcagagcctgcacagcttccacaccctgttctgccggagatgcttcaaatacgactgcttcctgcaccccttccacgccaccccgaacacctacaagcgcaagaacaccgaaaccgccctggacaacaagccgtgcggaccccaatgctaccagcacctggaaggcgccaaggagttcgccgcagcactgaccgccgagcggatcaaaaccccgccgaaaagacccggaggcagacggagagggagactcccaaacaacagcagcagaccgagcacgccgaccatcaacgtgctcgagagcaaggacaccgacagcgacagggaagcgggaaccgaaaccggaggagaaaacaacgacaaggaggaggaagaaaagaaggacgagacaagcagcagcagcgaagccaacagcagatgccaaaccccgatcaagatgaagccgaacatcgagccgccggagaacgtggaatggagcggagccgaagcgagcatgttccgggtgctgatcggcacctactacgacaacttctgcgccatcgcacgcctgatcggaaccaagacgtgccgccaggtctacgagttccgcgtcaaggagagcagcatcatcgcaccagcaccagccgaggacgtcgacaccccaccacggaagaagaagcggaaacacagactctgggccgcacactgcagaaagatccagctgaagaaggacggaagcagcaaccacgtgtacaactaccaaccatgcgaccacccgagacaaccctgcgacagcagctgcccctgcgtgatcgcccagaacttctgcgaaaagttctgccagtgcagcagcgagtgccagaacaggttcccaggatgccgctgcaaggcccagtgcaacaccaagcaatgcccatgctacctcgccgtgagggagtgcgacccggacctgtgcctcacatgcggagcagcggaccactgggacagcaagaacgtcagctgcaagaactgcagcatccagagaggaagcaagaagcacctcctgctggcgccaagcgacgtggccggatggggaatcttcatcaaggacccagtccaaaagaacgaattcatcagcgaatactgcggagaaatcatcagccaggacgaagccgaccgcagagggaaggtgtacgacaagtacatgtgcagcttcctcttcaacctgaacaacgacttcgtagtggacgccaccagaaagggcaacaagatccgcttcgccaaccacagcgtgaacccgaactgctacgcgaaggtcatgatggtgaacggcgaccaccggatcgggatcttcgccaagcgggccatccaaaccggagaggagctgttcttcgactaccggtacagccaagcggacgccctgaaatacgtgggcatcgagcgggagatggagatccca

SEQ ID NO: 75 - DNA, 1155 bp, Spiroplasma monobiae
  misc_feature 1..1155: note = MQ1 (P15840.3), Effector DNA sequence
  source 1..1155: mol_type = genomic DNA
agcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggc

SEQ ID NO: 76 - protein, 376 aa, Homo sapiens
  SITE 1..376: note = HDAC8 (NP_060956.1), Effector Protein Sequence
EEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVV

SEQ ID NO: 77 - protein, 382 aa, Homo sapiens
  SITE 1..382: note = G9A (NP_006700.3), Effector Protein Sequence
GSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 78 - protein, 745 aa, Homo sapiens
  SITE 1..745: note = EZH2 (NP_001190176.1), Effector Protein Sequence
GQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 79 - protein, 385 aa, Spiroplasma monobiae
  SITE 1..385: note = MQ1 (P15840.3), Effector Protein Sequence
SKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 80 - RNA, 1128 nt, Homo sapiens
  misc_feature 1..1128: note = HDAC8 (NP_060956.1), Effector RNA sequence
  source 1..1128: mol_type = genomic RNA
gaagaacctgaggagcctgccgattccggacagagcctggtgcccgtgtatatctactcccccgaatacgtgtctatgtgcgacagcttggctaagattccgaagcgggcatcaatggtgcactcgctgatcgaggcctacgccctgcacaagcagatgagaatcgtgaagcccaaggtcgcctcaatggaagagatggccaccttccacaccgacgcctacttgcaacatctccaaaaagtgtcccaggaaggggacgacgaccaccctgactccattgagtacggcctgggttacgactgcccagctaccgaagggatcttcgactacgccgctgccattggcggagcgaccatcactgccgcgcagtgcctcatcgacggaatgtgcaaggtcgccatcaactggtccggaggctggcaccacgccaagaaggatgaagcctccggcttctgctaccttaacgacgccgtgctgggaatccttcgcctgcgaagaaagttcgaacggattttgtatgtggacctggatctgcatcatggagatggcgtggaggatgccttctccttcacctcgaaagtcatgaccgtgtcacttcacaagttctcacccggcttcttccctgggaccggagatgtgtccgacgtcggtctgggaaagggccgctactactccgtgaacgtgccgatccaggacggtatccaggacgagaagtactaccagatctgcgagagcgtgctgaaggaagtgtaccaggccttcaatcccaaggccgtggtgctgcagcttggcgcggacactattgcgggcgacccgatgtgctccttcaacatgacccccgtcggtattggaaagtgcctgaagtacatcctgcaatggcagctggctaccctgatcctgggaggaggcggatacaacctcgcaaacaccgcccggtgttggacttacctgactggggtcatcctgggaaagactctgtcatccgagattcccgaccacgagttcttcaccgcgtacggcccagattacgtgctcgaaatcactccttcctgccggcctgatagaaatgagccgcaccgcatacagcagatcctcaactacattaagggcaacctgaagcacgtggtg

SEQ ID NO: 81 - RNA, 1146 nt, Homo sapiens
  misc_feature 1..1146: note = G9A (NP_006700.3), Effector RNA sequence
  source 1..1146: mol_type = genomic RNA
ggaagcgccgctatcgccgaagtgctgctgaatgccagatgcgatctgcatgccgtgaactaccacggcgacacccctctgcatatcgccgctagagagagctaccatgactgtgtgctgctgtttctgagcagaggcgccaatcccgagctcagaaacaaagagggcgacaccgcttgggatctgacacccgagagatccgacgtgtggttcgctctgcaactgaatagaaaactgagactgggcgtcggcaatagggccattagaaccgagaagatcatctgtagggacgtggctaggggctacgagaacgtgcccatcccttgtgtgaacggagtggatggagagccttgccccgaggattacaaatacatcagcgagaactgcgaaacctccaccatgaatatcgatagaaacattacacacctccagcactgtacatgcgtggacgattgcagcagcagcaactgtctgtgcggccaactgagcatcagatgctggtacgacaaggatggcagactgctgcaagagttcaacaagatcgaaccccctctgatcttcgagtgtaaccaagcttgcagctgttggaggaactgcaagaatagggtcgtgcagtccggaatcaaggtgagactgcagctgtatagaacagctaagatgggatggggagtcagagctctgcagaccatcccccaaggcacattcatctgtgagtacgtcggcgaactcatcagcgacgctgaggccgatgtgagggaggacgacagctatctcttcgacctcgacaacaaggacggcgaggtgtactgcatcgacgctagatattacggcaacatcagcagattcatcaaccacctctgcgaccccaatatcatccccgtgagagtgttcatgctccatcaagatctgagattccctaggatcgccttcttcagctctagagacattagaaccggcgaggagctgggattcgactacggcgacaggttctgggacatcaagagcaagtacttcacatgccaatgcggcagcgagaaatgcaagcatagcgccgaggccattgctctggagcagtctagactggctaggctggaccctcaccccgagctgctgcccgaactgggatctctgcctcccgtgaatacc

SEQ ID NO: 82 - RNA, 2235 nt, Homo sapiens
  misc_feature 1..2235: note = EZH2 (NP_001190176.1), Effector RNA sequence
  source 1..2235: mol_type = genomic RNA
ggacagacgggcaaaaagagcgaaaagggacccgtctgctggcggaaacgagtgaaaagcgaatacatgcgcctgcggcaactcaagaggttccgcagagccgacgaggtcaagagcatgttcagcagcaacagacaaaagatcctggagaggaccgaaatcctcaaccaggaatggaagcagcgcaggatccagccggtgcacatcctgaccagcgtcagcagcctcagaggcacccgcgagtgcagcgtgaccagcgacctggacttccccacccaagtgatcccgctgaaaaccctcaacgcagtggccagcgtgccgatcatgtacagctggagccccctgcagcagaacttcatggtcgaggacgaaaccgtgctgcacaacatcccgtacatgggcgacgaagtgctggaccaggacggaaccttcatcgaggaactgatcaagaactacgacggaaaggtgcacggggacagggaatgcgggttcatcaacgacgagatcttcgtcgagctcgtcaacgccctgggacaatacaacgacgacgacgacgacgacgacggggacgacccagaagaacgggaggaaaagcagaaggacctggaggaccaccgcgacgacaaggaaagcagaccccccaggaagttcccgagcgacaagatcttcgaagccatcagcagcatgttcccggacaagggaaccgccgaggagctgaaggaaaagtacaaggaactgaccgagcagcagctcccaggagcactgccgccggaatgcacaccaaacatcgacggaccgaacgccaagagcgtgcagagggagcagagcctgcacagcttccacaccctgttctgccggagatgcttcaaatacgactgcttcctgcaccccttccacgccaccccgaacacctacaagcgcaagaacaccgaaaccgccctggacaacaagccgtgcggaccccaatgctaccagcacctggaaggcgccaaggagttcgccgcagcactgaccgccgagcggatcaaaaccccgccgaaaagacccggaggcagacggagagggagactcccaaacaacagcagcagaccgagcacgccgaccatcaacgtgctcgagagcaaggacaccgacagcgacagggaagcgggaaccgaaaccggaggagaaaacaacgacaaggaggaggaagaaaagaaggacgagacaagcagcagcagcgaagccaacagcagatgccaaaccccgatcaagatgaagccgaacatcgagccgccggagaacgtggaatggagcggagccgaagcgagcatgttccgggtgctgatcggcacctactacgacaacttctgcgccatcgcacgcctgatcggaaccaagacgtgccgccaggtctacgagttccgcgtcaaggagagcagcatcatcgcaccagcaccagccgaggacgtcgacaccccaccacggaagaagaagcggaaacacagactctgggccgcacactgcagaaagatccagctgaagaaggacggaagcagcaaccacgtgtacaactaccaaccatgcgaccacccgagacaaccctgcgacagcagctgcccctgcgtgatcgcccagaacttctgcgaaaagttctgccagtgcagcagcgagtgccagaacaggttcccaggatgccgctgcaaggcccagtgcaacaccaagcaatgcccatgctacctcgccgtgagggagtgcgacccggacctgtgcctcacatgcggagcagcggaccactgggacagcaagaacgtcagctgcaagaactgcagcatccagagaggaagcaagaagcacctcctgctggcgccaagcgacgtggccggatggggaatcttcatcaaggacccagtccaaaagaacgaattcatcagcgaatactgcggagaaatcatcagccaggacgaagccgaccgcagagggaaggtgtacgacaagtacatgtgcagcttcctcttcaacctgaacaacgacttcgtagtggacgccaccagaaagggcaacaagatccgcttcgccaaccacagcgtgaacccgaactgctacgcgaaggtcatgatggtgaacggcgaccaccggatcgggatcttcgccaagcgggccatccaaaccggagaggagctgttcttcgactaccggtacagccaagcggacgccctgaaatacgtgggcatcgagcgggagatggagatccca

SEQ ID NO: 83 - RNA, 1155 nt, Spiroplasma monobiae
  misc_feature 1..1155: note = MQ1 (P15840.3), Effector RNA sequence
  source 1..1155: mol_type = genomic RNA
agcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggc

SEQ ID NO: 84-158 - skipped (75 entries)

SEQ ID NO: 159 - protein, 1820 aa, synthetic construct
  REGION 1..1820: note = Synthetic: >Amino acid sequence of the Effector Fusion Protein of dCas9-MQ1
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 160 - protein, 1414 aa, synthetic construct
  REGION 1..1414: note = Synthetic: >Amino acid sequence of the Effector Fusion Protein of dCas9
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKGSYPYDVPDYA

SEQ ID NO: 161 - protein, 1528 aa, synthetic construct
  REGION 1..1528: note = Synthetic: >Amino acid sequence of the Effector Fusion Protein of dCas9-KRAB
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 162 - protein, 1708 aa, synthetic construct
  REGION 1..1708: note = Synthetic: >Amino acid sequence of the Effector Fusion Protein of G9A-dCas9
MAPKKKRKVGIHGVPAASGGGGSGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDGGGGSGDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKGSYPYDVPDYA

SEQ ID NO: 163 - protein, 2177 aa, synthetic construct
  REGION 1..2177: note = Synthetic: >Amino acid sequence of the Effector Fusion Protein of EZH2-dCas9
MAPKKKRKVGGSGGSGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKGSYPYDVPDYA

SEQ ID NO: 164 - protein, 1818 aa, synthetic construct
  REGION 1..1818: note = Synthetic: >Amino acid sequence of the Effector Fusion Protein of dCas9-HDAC8
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKSGGGGSEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVVGGGGSGKRPAATKKAGQAKKKKGSYPYDVPDYA

SEQ ID NO: 165 - RNA, 5705 nt, synthetic construct
  misc_feature 1..5705: note = Synthetic: > Nucleotide sequence of the Effector Fusion Protein of dCas9-MQ1
  source 1..5705: mol_type = other RNA
aaggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaaggcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 166 - RNA, 4487 nt, synthetic construct
  misc_feature 1..4487: note = Synthetic: >Nucleotide sequence of the Effector Fusion Protein of dCas9
  source 1..4487: mol_type = other RNA
aaggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 167 - RNA, 4829 nt, synthetic construct
  misc_feature 1..4829: note = Synthetic: >Nucleotide sequence of the Effector Fusion Protein of dCas9-KRAB
  source 1..4829: mol_type = other RNA
aaggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaaggccagcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 168 - RNA, 5368 nt, synthetic construct
  misc_feature 1..5368: note = Synthetic: > Nucleotide sequence of the Effector Fusion Protein of G9A-dCas9
  source 1..5368: mol_type = other RNA
aggaaataagagagaaaagaagagtaagaagaaatataagagccaccatggcccccaagaagaagcggaaggtgggcatccacggcgtgcccgccgcctcgggcgggggtggctcaggaaatagggctatcagaaccgagaagatcatctgtagggacgtggctagaggctacgagaacgtgcccattccttgcgtgaatggcgtggatggcgaaccttgccccgaggactacaaatacatctccgagaactgcgaaaccagcacaatgaacatcgacagaaacatcacccacctccagcactgcacatgtgtggatgactgctcctccagcaactgtctgtgcggccagctctccatcagatgctggtacgacaaggacggcagactgctgcaagagttcaacaagatcgaaccccctctcatcttcgagtgtaaccaagcttgcagctgctggagaaactgcaagaatagagtggtccagagcggcatcaaggtgagactgcaactgtacagaaccgccaagatgggatggggagtgagggctctgcaaaccattccccaaggcaccttcatctgcgaatacgtgggcgaactgatctccgacgccgaagctgacgtgagagaggacgacagctatctcttcgatctggacaataaggacggcgaggtgtactgcatcgacgctagatattacggcaacatctctagattcatcaaccacctctgcgatcccaacatcattcccgtgagggtgttcatgctgcaccaagatctgaggttccctagaatcgccttcttcagctctagagacatcagaaccggcgaggagctgggcttcgattacggcgatagattctgggacatcaagtccaagtacttcacatgccagtgcggcagcgagaagtgtaagcacagcgctgaggccattgctctggagcagtctagactggccagactggatggaggtggcggatcgggagacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 169 - RNA, 6775 nt, synthetic construct
  misc_feature 1..6775: note = Synthetic: >Nucleotide sequence of the Effector Fusion Protein of EZH2-dCas9
  source 1..6775: mol_type = other RNA
aggaaataagagagaaaagaagagtaagaagaaatataagagccaccatggcccccaagaagaagcggaaggtgggcggcagcggcggcagcggccagaccggcaagaagagcgagaagggccccgtgtgctggcggaagcgggtgaagagcgagtacatgcggctgcggcagctgaagcggttccggcgggccgacgaggtgaagagcatgttcagcagcaaccggcagaagatcctggagcggaccgagatcctgaaccaggagtggaagcagcggcgaatccagcccgtgcacatcctgaccagcgtgagcagcctgcggggcacccgggagtgcagcgtgaccagcgacctggacttccccacccaggtgatccccctaaagaccctgaacgccgtggccagcgtgcccatcatgtacagctggagccccctgcagcagaacttcatggtggaggacgagaccgtgctgcacaacatcccctacatgggcgacgaggtgctggaccaggacggcaccttcatcgaggagctgatcaagaactacgacggcaaggtgcacggcgaccgggagtgcggcttcatcaacgacgagatcttcgtggagctggtgaacgccctgggccagtacaacgacgacgacgacgacgacgacggcgacgaccccgaggagcgggaggagaagcagaaggacctggaggaccaccgggacgacaaggagagccggcccccccggaagttccccagcgacaagatcttcgaggccatcagcagcatgttccccgacaagggcaccgccgaggagctgaaggagaagtacaaggagctgaccgagcagcagctgcccggcgccctgccccccgagtgcacccccaacatcgacggccccaacgccaagagcgtgcagcgggagcagagcctgcacagcttccacaccctgttctgccggcggtgcttcaagtacgactgcttcctgcaccccttccacgccacccccaacacctacaagcggaagaacaccgagaccgccctggacaacaagccctgcggcccccagtgctaccagcacctggagggcgccaaggagttcgccgccgccctgaccgccgagcggatcaagaccccccccaagcggcccggcggccggcggcggggccggctgcccaacaacagcagccggcccagcacccccaccatcaacgtgctggagagcaaggacaccgacagcgaccgggaggccggcaccgagaccggcggcgagaacaacgacaaggaggaggaggagaagaaggacgagaccagcagcagcagcgaggccaacagccggtgccagacccccatcaagatgaagcccaacatcgagccccccgagaacgtggagtggagcggcgccgaggccagcatgttccgggtgctgatcggcacctactacgacaacttctgcgccatcgcccggctgatcggcaccaagacctgccggcaggtgtacgagttccgggtgaaggagagcagcatcatcgcccccgcccccgccgaggacgtggacacccccccccggaagaagaagcggaagcaccggctgtgggccgcccactgccggaagatccagctgaagaaggacggcagcagcaaccacgtgtacaactaccagccctgcgaccacccccggcagccctgcgacagcagctgcccctgcgtgatcgcccagaacttctgcgagaagttctgccagtgcagcagcgagtgccagaaccggttccccggctgccggtgcaaggcccagtgcaacaccaagcagtgcccctgctacctggccgtgcgggagtgcgaccccgacctgtgcctgacctgcggcgccgccgaccactgggacagcaagaacgtgagctgcaagaactgcagcatccagcggggcagcaagaagcacctgctgctggcccccagcgacgtggccggctggggcatcttcatcaaggaccccgtgcagaagaacgagttcatcagcgagtactgcggcgagatcatcagccaggacgaggccgaccggcggggcaaggtgtacgacaagtacatgtgcagcttcctgttcaacctgaacaacgacttcgtggtggacgccacccggaagggcaacaagatccggttcgccaaccacagcgtgaaccccaactgctacgccaaggtgatgatggtgaacggcgaccaccggatcggcatcttcgccaagcgggccatccagaccggcgaggagctgttcttcgactaccggtacagccaggccgacgccctgaagtacgtgggcatcgagcgggagatggagatccccagcaccggcggcagcggcggcagcggcggcagcggcggcagcggcggcagcggccgacccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 170 - RNA, 5698 nt, synthetic construct
  misc_feature 1..5698: note = Synthetic: >Nucleotide sequence of the Effector Fusion Protein of dCas9-HDAC8
  source 1..5698: mol_type = other RNA
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagtcgggcgggggtggctcagaggagcccgaggagcccgccgatagcggacaatctctggtgcccgtctacatctacagccccgaatatgtgagcatgtgtgattccctcgccaagatccctaagagagccagcatggtgcattctctgatcgaggcctacgctctgcataagcaaatgaggatcgtgaagcccaaggtcgccagcatggaagagatggccacctttcacaccgatgcctacctccaacatctccagaaggtgtcccaagagggcgacgacgaccaccccgactccattgagtacggactgggctatgattgccccgccaccgagggcatctttgactatgccgccgctatcggcggagctaccatcacagccgcccagtgtctgattgatggcatgtgcaaggtcgccatcaactggtccggaggctggcatcatgccaagaaggatgaggcctccggcttctgttatctgaatgacgccgtgctgggcattctgagactgaggaggaaattcgagaggattctgtacgtggatctggatctgcatcacggagatggagtcgaagatgccttcagcttcaccagcaaggtgatgacagtctctctgcacaagttctcccccggcttctttcccggaaccggcgacgtgtccgacgtgggactgggcaagggaaggtactacagcgtgaacgtgcccattcaagacggcatccaagacgagaagtactaccagatctgcgagtccgtgctcaaggaggtctaccaagccttcaatcctaaggctgtcgtgctccaactgggagctgataccattgctggcgatcccatgtgcagcttcaatatgacacccgtcggaatcggcaagtgcctcaagtacatcctccagtggcagctcgccaccctcattctcggaggaggcggatacaatctggctaataccgccagatgctggacctatctgaccggcgtgattctgggcaaaacactgagcagcgaaatccccgaccacgagtttttcaccgcttacggccccgactacgtgctggagatcacccccagctgcagacccgatagaaacgaaccccatagaatccagcaaattctgaactatatcaagggcaacctcaagcacgtcgtgggaggtggcggatcgggaaagcggcccgccgccaccaagaaggccggtcaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 171 - protein, 1367 aa, synthetic construct
  REGION 1..1367: note = Synthetic: >Amino acid Sequence of DNA Binding Domain of the Effector Fusion Protein of dCas9-MQ1
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 172-296 - skipped (125 entries)

SEQ ID NO: 297 - protein, 96 aa, synthetic construct
  REGION 1..96: note = Synthetic: KRAB, Effector Protein Sequence
DAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 298 - RNA, 288 nt, synthetic construct
  misc_feature 1..288: note = Synthetic: KRAB, Effector RNA sequence
  source 1..288: mol_type = other RNA
gacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtg

SEQ ID NO: 299 - protein, 1367 aa, synthetic construct; identical to SEQ ID NO: 171
  REGION 1..1367: note = Synthetic: >dCas9 amino acid sequence

SEQ ID NO: 300 - RNA, 4101 nt, synthetic construct
  misc_feature 1..4101: note = Synthetic: >dCas9 mRNA sequence
  source 1..4101: mol_type = other RNA
gacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgac

SEQ ID NO: 301 - skipped

SEQ ID NO: 302 - DNA, 288 bp, synthetic construct
  misc_feature 1..288: note = Synthetic: KRAB, Effector DNA sequence
  source 1..288: mol_type = other DNA
gacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtg

SEQ ID NO: 303-308 - skipped (6 entries)

SEQ ID NO: 309 - protein, 7 aa, synthetic construct
  REGION 1..7: note = Synthetic
LEPGEKP

SEQ ID NO: 310 - protein, 6 aa, synthetic construct
  REGION 1..6: note = Synthetic
TGKKTS

SEQ ID NO: 311 - protein, 12 aa, synthetic construct
  REGION 1..12: note = Synthetic
THPRAPIPKPFQ

SEQ ID NO: 312 - protein, 16 aa, synthetic construct
  REGION 1..16: note = Synthetic
TPNPHRRTDPSHKPFQ

SEQ ID NO: 313-399 - skipped (87 entries)

SEQ ID NO: 400 - protein, 204 aa, synthetic construct
  REGION 1..204: note = Synthetic: DBD386, ZF24
LEPGEKPYKCPECGKSFSSKKHLAEHQRTHTGEKPYKCPECGKSFSQNSTLTEHQRTHTGEKPYKCPECGKSFSQSGDLRRHQRTHTGEKPYKCPECGKSFSTHLDLIRHQRTHTGEKPYKCPECGKSFSDPGNLVRHQRTHTGEKPYKCPECGKSFSTSGNLTEHQRTHTGEKPYKCPECGKSFSERSHLREHQRTHTGKKTS

SEQ ID NO: 401 - DNA, 612 bp, synthetic construct
  misc_feature 1..612: note = Synthetic: DBD386, ZF24
  source 1..612: mol_type = other DNA
ctggaacccggagagaagccctataagtgtccggaatgcggaaagagtttcagctcgaagaagcacctcgccgagcaccaacggacccacacgggagaaaagccctacaagtgcccagagtgcggaaagtcgttcagccaaaactccaccctgaccgaacaccagcggactcataccggggagaaaccttacaagtgtccagaatgcgggaagtcgttctcccaatccggagacttgcgcagacaccagcgcactcacaccggcgaaaaaccgtacaaatgcccggagtgtggaaagtcattttccacccacctggatctgatcaggcatcagcgcacccacactggcgaaaagccttacaaatgtccggagtgcggcaaatccttctctgaccccggcaacctcgtgcgccatcagaggactcacactggggagaagccttataagtgccccgaatgtggaaagagcttctccacctccggcaaccttaccgagcatcagcggacacacaccggggaaaagccgtacaagtgccctgagtgtggcaaatcattctccgaacggagccatctgcgggagcaccagagaacccacaccggaaagaaaacttcc

SEQ ID NO: 402 - protein, 204 aa, synthetic construct
  REGION 1..204: note = Synthetic: DBD374, ZF16
LEPGEKPYKCPECGKSFSQRAHLERHQRTHTGEKPYKCPECGKSFSRSDHLTNHQRTHTGEKPYKCPECGKSFSRNDALTEHQRTHTGEKPYKCPECGKSFSRADNLTEHQRTHTGEKPYKCPECGKSFSSKKALTEHQRTHTGEKPYKCPECGKSFSRNDALTEHQRTHTGEKPYKCPECGKSFSHKNALQNHQRTHTGKKTS

SEQ ID NO: 403 - DNA, 612 bp, synthetic construct
  misc_feature 1..612: note = Synthetic: DBD374, ZF16
  source 1..612: mol_type = other DNA
ctggagccgggagagaagccttataagtgtcctgagtgcggcaaatcattctcccaacgcgcccacctcgagagacaccagagaacccacaccggggaaaagccgtacaagtgccctgagtgtgggaagtccttttcccggtccgaccatttgaccaaccatcagcggacccacactggggagaagccatacaaatgcccagaatgcggcaaaagcttctcccggaacgacgcacttaccgaacatcagcgcacgcacaccggagagaaaccctataagtgccccgaatgtggaaagtcattctcccgggccgacaacctgacagagcaccagcggactcacactggagaaaagccttacaagtgtcccgaatgcgggaagtcgttcagctcgaagaaggctctgaccgaacaccagaggacccataccggcgaaaagccctataaatgcccggagtgcggaaagagcttcagcaggaacgatgccctcaccgagcaccaacgcactcatactggcgaaaaaccgtacaaatgtccggaatgcggaaagtccttctcgcacaagaacgcgctgcagaatcaccagcgcactcacaccggcaaaaagacctct

SEQ ID NO: 404 - protein, 204 aa, synthetic construct
  REGION 1..204: note = Synthetic: DBD360, ZF07
LEPGEKPYKCPECGKSFSDKKDLTRHQRTHTGEKPYKCPECGKSFSQSSSLVRHQRTHTGEKPYKCPECGKSFSTTGNLTVHQRTHTGEKPYKCPECGKSFSTSGELVRHQRTHTGEKPYKCPECGKSFSTTGNLTVHQRTHTGEKPYKCPECGKSFSTSHSLTEHQRTHTGEKPYKCPECGKSFSTSGNLVRHQRTHTGKKTS

SEQ ID NO: 405 - DNA, 612 bp, synthetic construct
  misc_feature 1..612: note = Synthetic: DBD360, ZF07
  source 1..612: mol_type = other DNA
ctcgagcctggcgaaaagccttataagtgcccagaatgcggaaagagcttctccgacaagaaggatctgacccgccaccaaaggacccacactggggagaagccttacaagtgtccggagtgcggaaagtccttttcgcaatcgtccagcctggtccggcaccagaggacgcacaccggagagaagccatacaaatgcccggagtgtgggaagtcgttctccaccactggaaacctcactgtgcaccagcgcacgcatactggggaaaagccctataagtgtccagagtgcgggaagtccttctcaacctccggcgaattggtcagacaccagcggacacacaccggcgagaagccctacaagtgccctgagtgcggcaaaagcttcagcactaccggcaacctgactgtgcatcagcggacccataccggagaaaaaccctacaaatgtcccgaatgcggcaaatccttctccacctcccactcgctgaccgaacaccagagaacccatactggagaaaagccgtacaagtgcccggaatgtggaaagtctttcagcacctccggaaaccttgtgcggcatcagcgcactcacaccggcaaaaagacctca

SEQ ID NO: 406 - protein, 204 aa, synthetic construct
  REGION 1..204: note = Synthetic: DBD358, ZF05
LEPGEKPYKCPECGKSFSQSGHLTEHQRTHTGEKPYKCPECGKSFSQRANLRAHQRTHTGEKPYKCPECGKSFSTKNSLTEHQRTHTGEKPYKCPECGKSFSTSGSLVRHQRTHTGEKPYKCPECGKSFSQSGNLTEHQRTHTGEKPYKCPECGKSFSQAGHLASHQRTHTGEKPYKCPECGKSFSQSGDLRRHQRTHTGKKTS

SEQ ID NO: 407 - DNA, 612 bp, synthetic construct
  misc_feature 1..612: note = Synthetic: DBD358, ZF05
  source 1..612: mol_type = other DNA
ctggagcccggagagaaaccgtataagtgcccggaatgtggaaagtccttctcccaatccggccacctgactgagcatcagcgcacccacactggcgaaaagccgtacaagtgccctgagtgcgggaagtcgtttagccagcgggcgaacttgcgcgctcatcagcggactcataccggggagaagccctataaatgcccggagtgcggaaagagcttcagcaccaagaactccctgactgaacaccagcggacccacaccggagagaagccttacaagtgtcctgaatgcgggaaatccttctccacctccggttcactcgtgcgccaccagagaacgcacaccggtgaaaagccttataagtgtccagagtgcggcaaaagcttcagccagtccggcaacctcaccgagcaccaacggactcacactggagaaaagccctacaaatgtccggaatgcggcaaatcattctcgcaagccggtcaccttgcctcgcaccagcgcacacacaccggggaaaagccatacaaatgccccgaatgcggaaagtcgttctctcaatccggcgacctgagaaggcatcagaggacccataccggaaagaaaacctcc

SEQ ID NO: 408 - protein, 798 aa, Mus musculus
  SITE 1..798: note = DBD455, TAL37
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 409 - DNA, 2394 bp, Mus musculus
  misc_feature 1..2394: note = DBD455, TAL37
  source 1..2394: mol_type = genomic DNA
cacatggtggacctgagaactctgggttactcccagcaacagcaagaaaaaatcaagccaaaagtgcgcagcactgtcgcgcagcaccacgaggcccttgtggggcacgggttcacccacgctcacatcgtggccctgagccagcatccggcagccctgggaaccgtcgcagtgaagtaccaagacatgatcgccgccttgccggaagccactcacgaagcaattgtgggagtggggaagagaggagccggcgcaagagccctcgaagcgcttctgactgtggccggagaattgagaggccccccactgcagctggataccggacaactgctgaagatcgccaaacggggtggagtgaccgccgtggaagcggtgcacgcgtggagaaacgcgctgactggagccccgctcaacctgacccctgaacaggtcgtggctattgcctcaaacaacggcgggaagcaagcgttggagactgtacagcgcctgctgccagtgttgtgtcaagcccacgggctgactcccgagcaagtggtggccatagccagcaatatcggaggtaaacaggctctcgagactgtccaacgcctgctcccagtgctttgtcaggctcacggcctcactcctgaacaggtcgtagccattgccagcaacggtggcggcaaacaagccttagagactgtgcaacggctcttgccggtgctctgtcaggcccacggactgaccccagaacaagtagtcgctatcgcgtcaaacatcggcggcaagcaggctttggaaaccgtccagcggcttctgccggtgctgtgccaggcgcatgggctcacccccgaacaggtggtcgcaatcgcctcgaacggcggtggtaaacaagccctcgaaactgtccagcgcctcttgcccgtcctgtgtcaggcccatggcctgactccagaacaggtggtggcaatagcgtcccatgacgggggaaagcaagcccttgagactgtgcaacggttgctccccgtgttatgtcaggcacatggtctgacccccgaacaggtggtcgcaattgcgtcgaacggcggaggaaagcaagctctggaaacggtccaacggctgctccctgtgctgtgccaggcgcacggacttaccccagaacaagtggtcgccattgcgtccaacatcggagggaagcaggcgcttgaaaccgtgcagaggttgctgccggtgttatgccaagcacatggactaaccccggagcaagtggtggccattgcctcgaatattggggggaaacaagcgttagaaactgtgcagaggctgctgcctgtgctctgccaagcccatggtcttaccccggaacaggttgtggcgattgcatccaacaacggtggaaaacaggcccttgaaacggtgcagcggctccttcctgtcctgtgccaagcccatggactcacccctgaacaggtcgtggctattgcctccaacggaggcggcaagcaggccctggagacagtccagagactgctgcccgtgctgtgtcaagctcacggtctgactccggaacaggtggttgctatcgcttcgcacgacggcggaaaacaagctcttgaaaccgtgcaaaggctgctcccggtgctttgccaagctcatggcttgaccccagagcaagtcgtggcaatcgcgtccaatatcggtgggaagcaggctcttgagacagtgcaaagactcctccccgtcctctgtcaagcgcacgggctcactcctgaacaggtcgtcgctattgcttcgaacaacgggggcaaacaggcactagagactgtccaaagacttttacctgtcctctgccaggcacacggattgactcccgaacaagtcgtcgcgattgcctcaagacacgggggcaagcaagcgctggagactgttcagcgactgcttcctgtgctttgccaggcccacggtttgacccctgagcaagttgtggccatcgcctctaacggaggtggaaagcaggccttggaaacagtgcagagactgttgcccgtcttgtgccaggctcatgggcttacgcctgaacaagtcgtcgccatcgcttcgaacggaggagggaaacaggcgctggaaactgtgcagcgactccttccggtcctttgtcaagcacacggccttacgccggaacaagtggttgccatcgcaagccacgacggaggacgccctgccctcgagtccattgtggcccagctgtcacgcccagatcctgcactggccgctcttaccaacgatcacctggtcgctctcgcgtgcctgggaggaaggcctgccctggatgccgtcaagaagggactgcctcatgcccctgctttgattaagaggactaatcggcgcatccccgaacggaccagccacagggtg

SEQ ID NO: 410 - protein, 798 aa, Mus musculus
  SITE 1..798: note = DBD452, TAL34
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 411 - DNA, 2394 bp, Mus musculus
  misc_feature 1..2394: note = DBD452, TAL34
  source 1..2394: mol_type = genomic DNA
cacatggtggacctgagaactctgggttactcccagcaacagcaagaaaaaatcaagccaaaagtgcgcagcactgtcgcgcagcaccacgaggcccttgtggggcacgggttcacccacgctcacatcgtggccctgagccagcatccggcagccctgggaaccgtcgcagtgaagtaccaagacatgatcgccgccttgccggaagccactcacgaagcaattgtgggagtggggaagagaggagccggcgcaagagccctcgaagcgcttctgactgtggccggagaattgagaggccccccactgcagctggataccggacaactgctgaagatcgccaaacggggtggagtgactgccgtcgaggctgtgcacgcctggagaaacgccttgaccggagcgccactgaaccttaccccggagcaagtggtggcgatagcatcaaatattggcggaaagcaggccctggaaaccgtacaaagactgttgccggtcctgtgtcaagctcatggtttgaccccagaacaggtcgtggctattgcatcgaacggcggaggcaaacaagccctcgagacagtccagagacttctgccggttctgtgccaggcgcacggacttacccctgaacaggtggtggcgattgcgagccacgacggtggtaaacaggcgttggaaactgtgcagcggttgcttccggtgctgtgtcaggctcacgggctgacccccgaacaagtggtagctatcgcctcccacgacggagggaagcaggctctggaaacggtgcaacgcctccttcccgtcttgtgccaagcacatggactgactccagaacaggtcgtcgccattgcctcccatgatggggggaagcaggcactggaaactgtacaacgcctgctgcctgtcctctgccaagcgcacggtttgacgccggaacaggttgtcgccatcgcctcgaacggagggggaaaacaggcccttgaaacagtgcaacggctgctgccagtgctgtgtcaggcgcatggcttgaccccggagcaagtggtggccatagcctcgaatggaggaggaaagcaggccctggaaaccgttcagcgcctcctccctgtgctgtgtcaggcccatgggctcactcccgaacaggtagtcgccatagcgtcgaacatcggaggaaaacaagcgctcgaaactgtccaaagactgcttccagtcctttgccaggctcatggactaactccagagcaagtagtggccatcgcgagccatgacggcggcaagcaagcactcgaaaccgtgcagaggctgcttcccgtcctgtgccaagctcacggcctgacgcctgaacaggtggtggctatcgccagcaacggtggtggaaagcaggcgctcgagactgtgcagaggttgctcccagtgctctgtcaggcacatggcctcaccccggagcaagtggtggcaatcgcctcaaacatcggtggcaaacaggcacttgaaaccgtgcagcgccttttgcctgtgctctgccaggcccacggactgacccctgagcaagtcgtggctattgcctctaacggcggtggcaagcaggccttggaaaccgtccagaggctcttgccggtgctctgccaggcacacggactcactccggaacaggtggtcgccatcgcatcgaatatcggcggaaaacaggccctcgaaacggtccagcggttactccccgtgctttgtcaggctcacggattgactcccgagcaagtcgtggccattgcttccaacattggggggaaacaggcgctggaaacggtgcagcgactccttcctgtgttgtgtcaagcccacggcctcactccagagcaagtcgtggcaattgcctccaacatcgggggaaagcaagccttggagacagtacagcggctgcttcctgtgctttgccaagcccatggactcacccccgaacaagtggttgccattgccagccacgatggagggaaacaagccctggagacagtgcaaaggctcctgcccgtgctgtgccaggcacacggcttgactcctgaacaagtcgtcgctatcgcttccaatggtggtgggaagcaagctctggagactgtccagcggctcctgccagtgttgtgtcaagcacacggtctgaccccggaacaagtcgttgcaatcgcgtcgcatgatggaggacgccctgccctcgagtccattgtggcccagctgtcacgcccagatcctgcactggccgctcttaccaacgatcacctggtcgctctcgcgtgcctgggaggaaggcctgccctggatgccgtcaagaagggactgcctcatgcccctgctttgattaagaggactaatcggcgcatccccgaacggaccagccacagggtg

SEQ ID NO: 412 - protein, 798 aa, Mus musculus
  SITE 1..798: note = DBD429, TAL27
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRV

SEQ ID NO: 413 - DNA, 2394 bp, Mus musculus
  misc_feature 1..2394: note = DBD429, TAL27
  source 1..2394: mol_type = genomic DNA
cacatggtggacctccgcacgctgggatactcgcaacagcagcaggagaagattaagcctaaggtccggtccaccgtggctcagcaccatgaggcgctagtgggccacggattcacccacgcgcatattgtcgccctctcgcaacatcccgccgcactgggaaccgtcgccgtgaagtaccaggacatgattgccgccctgcctgaagctacccatgaagccattgtgggcgtgggaaagagaggagcaggggccagagccttggaggccctgctcacggtagccggagagctcagaggcccaccactgcagcttgacaccggccaacttctgaagatcgccaagcggggaggagtgaccgccgtggaggcagtgcatgcctggcgcaacgccttgactggagccccgctgaacctgactccggagcaagtggtggctatcgcgtccaacatcggaggaaaacaggccctggaaacggtgcaaagactgctgccggtcctctgtcaagcgcacggactcactcccgagcaagttgtggctatcgcttccaacatcggggggaaacaggctctggaaactgtccaaaggctgttacctgtgctgtgccaagcacacggactgaccccagagcaagtggtcgccatagcctccaacaacggggggaagcaagccctggagactgtgcagcggttgctccccgtcctgtgccaggcccacggattgactcccgaacaagtggtggccattgcgtcaaacatcgggggcaagcaagcgttggaaacagtccaacggttgcttcccgtcctttgccaggcccacggtctgaccccggagcaagtcgtcgccattgctagtaacgggggaggaaagcaagctcttgaaaccgtccagcggcttcttccggtgctctgccaagcacacggcctcacacccgagcaagtagtggccattgcctcaaacattgggggaaagcaggcattggaaactgtgcaacggctgcttcctgtcttgtgccaggcgcatgggctgactccagaacaggtggtcgctatcgcgagtaacaacggtggcaaacaggcactggaaaccgtgcagaggctcttgcctgtcctgtgtcaggcacacggactgacgccagaacaggtggtcgctattgcgagcaatatcggcggaaagcaggcgttagaaactgtgcagagactgctcccagtgctatgccaggctcatgggttgactcctgaacaagtggtggctatcgcctcaaacggtggcggtaaacaggcgcttgagacagtgcaaaggctccttcccgtgttgtgccaagcgcatggcctgacccctgaacaggtcgtggctatcgcgtcaaacaacggaggcaagcaggccctagaaacggtacaacggctgctgcccgtactgtgccaggcccacggactgaccccagaacaggtggtcgcaattgcctccaacattggcgggaagcaggcgctagaaaccgtgcagcggcttctgcccgtcctctgccaggcccacggtctgactcctgagcaagtcgtcgcaatcgccagcaacatcggcggaaaacaagccctcgaaaccgtccagcgactgctgcctgtcctatgccaggcccacgggttgaccccggaacaggtcgtcgccattgcttccaataatggaggcaaacaggcactcgaaacggtgcagagacttctgccggtgctgtgtcaagctcacggcttgacccccgagcaagtggtggcaatcgcttcacgacacggtggaaaacaggccctggagacagtccagagactgctccccgtgctttgccaagctcacggacttactccggaacaagtcgtggcgattgcgtccaacggtggtggaaagcaggccttggaaacggtccagagactgttgccggtcttgtgccaggcgcatggtctgacccccgaacaagtcgtggcaattgcatcccatgacggcgggaaacaggctttggagactgtccagcgcctgttgccagtcctttgtcaagcccacgggttgacacccgaacaggtcgtcgcaatcgcctcgaacatcggtggcaagcaggcccttgaaactgtccaacgcctcctcccggtgttgtgtcaggctcacggcctgactccagaacaagtcgtggccatagcgtcacacgacggtggacggccagccctggaatccatcgtggcgcagctgtccagacccgacccagcactggcggccctcactaacgatcaccttgtggccctggcctgtctgggaggtagaccagccctcgacgctgtgaagaagggactgccgcatgcaccagccctgatcaagcgaaccaaccggcgcattcccgagagaacttcacacagagtg

SEQ ID NO: 414 - protein, 798 aa, Homo sapiens
  SITE 1..798: note = DBD330, TAL14
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRV

SEQ ID NO: 415 - DNA, 2394 bp, Homo sapiens
  misc_feature 1..2394: note = DBD330, TAL14
  source 1..2394: mol_type = genomic DNA
cacatggtcgatcttcgcaccctcgggtactcgcagcagcaacaggaaaagatcaagccgaaagtcaggtccaccgtcgcccagcaccacgaggcactggtcggccacggattcacccacgcccacatcgtcgcgctgtctcaacatccagccgccctgggaactgtcgccgtcaagtaccaagatatgatcgcggcactgcctgaagcaacccatgaggccatcgtgggtgtgggaaagagaggagcaggggctagagccctggaagcgctcctgaccgtggcgggagagcttagaggccctcccctgcaattggacaccggccaacttctgaagattgcaaaaaggggtggagtgactgccgtggaagcagtgcacgcttggcggaacgcactgactggagcacccctgaatttgacccccgagcaagtggttgccatcgcaagcaacatcggagggaaacaagcactggaaactgtgcagcgcctgcttccggtcctgtgccaagctcacggactgactcctgaacaggttgtggccatcgcgtcgcatgacgggggaaaacaggcgctcgaaaccgtccaaagactgctcccggtgctgtgtcaggctcacggtttgaccccagaacaagttgtcgctatcgcctcacacgacggcgggaagcaagctctggagactgtgcaaagactccttcctgtgctgtgccaagctcatggattgaccccggaacaggtcgtcgccatcgcttccaatggcggaggtaaacaggccttggaaacggtgcagaggctgctgccagtcttgtgtcaggcgcacgggctgactccagagcaagtagtagccatcgccagcaatggaggcggtaaacaagcgctcgagaccgtccagcgcttgttgccagtgttgtgccaggcccatgggctgacgcccgaacaggtggtggccattgcatccaacattggggggaaacaggcgttggagacagtccaacggcttctgcccgtcctttgtcaggcacacggactcactcctgaacaagtcgttgccattgcctcgaatatcgggggtaagcaagccctcgagactgtgcagagattgttgcctgtcctgtgtcaagcgcatggtctgactcccgaacaggtggtggcgattgcttcgaacaatggaggaaaacaagcattggaaaccgtccagaggctgttaccagtcctttgccaggcacatggactgaccccggaacaggtcgtcgcaatcgcatcgaacattggaggcaagcaggctctcgaaacggtccagagactgctgcctgtgctctgccaagcccacggcctcactccggagcaagtcgtggcaatcgcctcaaacatcggtggaaagcaggcccttgagactgtccagcgactgctcccagtgctctgccaggcgcatggactcacacccgagcaggtggtcgccattgctagcaacaacggaggcaaacaagccctggagacagtgcaacgcttgctgcccgtgctttgtcaagcgcacggacttactccggaacaagtagtggctattgcgagtaggcacggcggtaagcaagccctggagacagtgcagcggcttttgccggtgctctgtcaggcccacgggctcacaccggaacaggtcgtagctattgcgtcccatgacggcggaaagcaagccttagaaactgtgcagcggttgctgcctgttctgtgccaggctcatgggctcactcccgagcaagtggtcgcgattgcgagcaacggaggtggcaagcaagcactagaaaccgtgcaacgactgctgccggtcttgtgccaagcgcatggcctgacacccgaacaggtggtggctatcgcgtccaacaacgggggcaaacaggccctggaaacagtccaaaggctgttgccggtcctctgtcaagctcacggactaacccccgaacaggtggtggcaatagcttcgcggcatgggggcaagcaggcactcgaaacagtgcagaggttgctcccggtactttgccaagcacatggcttgacccctgagcaagtcgtggccatcgcatccaacggcggagggaagcaagcgcttgaaactgtccaacgcctcctgcccgtgttgtgtcaagcccatggccttacgcctgaacaagtcgtcgctattgcaagtaacggaggagggaggcctgccctggagtccattgtcgcccaactgtcgcgacccgatccagccctggcagccctcactaacgaccatctcgtggcgctggcatgcctcggtggtagaccagctctggacgccgtgaagaagggacttcctcatgcccccgcactgatcaagcgcactaaccggaggatccctgaaagaacctcccacagagtg

SEQ ID NO: 416 - protein, 798 aa, Homo sapiens
  SITE 1..798: note = DBD203, TAL09
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRV

SEQ ID NO: 417 - DNA, 2394 bp, Homo sapiens
  misc_feature 1..2394: note = DBD203, TAL09
  source 1..2394: mol_type = genomic DNA
cacatggtggacctccggaccctgggatactcccagcagcagcaggaaaagatcaagccgaaggtccgctccactgtggctcaacaccacgaagcccttgtgggacacggcttcactcatgcacacattgtggcattgtcccaacaccctgccgctctgggaactgtggcagtgaagtaccaggacatgatagccgcactgcccgaagcaacacacgaggccatcgtcggtgtgggaaaaagaggagcgggagccagagcactcgaagcgctcctgacagtggcgggagaactccgcggaccacctctgcagctggacactggccagctgctgaagattgcaaaacgcggtggagtcaccgctgtggaggcggtgcatgcctggagaaatgccctgactggtgcccccttgaacctgacccccgagcaagtcgtcgcaattgcttcgaacaacggtggtaaacaggctctcgaaaccgtccaacggctgctcccagtcctatgccaagcccatggacttacgcccgaacaagtggttgctatagcctccaatatcggtgggaagcaggctctggagacagtccaaagactcctgccggtcctctgccaagcgcacggactcaccccagagcaagtagtggccattgcctcccacgatggtggcaagcaggcccttgaaacagtccagagactgttacccgtgctttgtcaagcccacgggctcactcccgaacaggtggtggcgattgcctctaacggcggcggcaaacaggcgctcgagactgtccagcggcttctaccggtgttgtgccaggcccatggtctgacacccgaacaggtggttgcaattgcctccaacggaggcgggaagcaagccctcgaaacggtccagcggttgttgccggtcctgtgtcaggcgcacgggttgaccccagaacaggtcgtcgctattgcctcaaacatcggtggtaagcaggcgctggaaactgtgcagcgactcctgcctgtgctctgtcaagcgcacgggcttacgccagaacaagtggtcgctatcgcgtccaacaacggcggcaagcaagctctggaaaccgtccaaaggctcttgcccgtcctatgtcaggcccacggcctcacccctgaacaggttgtcgccatcgccagccacgacggcggtaagcaagccttggaaaccgtgcaacgactgcttcccgtgttgtgtcaggctcatggcctcactcctgagcaagttgtggccatcgcttcgaatattgggggcaaacaagccctggaaacggtgcagcggttgctgcctgtcttgtgtcaagctcacgggctgactcctgaacaagtcgtggccatagcgtccaacattgggggaaagcaggcgttggaaactgtccagaggctgttgcccgtgctctgccaggctcacggcctgacccctgaacaggtggtcgcaatcgcctccaacatcggagggaagcaggcactggaaacagtgcagcggctgctgcccgtgctgtgccaagctcatggactcactccggaacaggtcgttgcaatcgcatcaaacattggaggaaagcaggccctcgagacagtgcagagactgctccctgtgctttgccaggcgcatggccttaccccggaacaagtggtagccatcgcgtcgcatgacggcggaaagcaggctttggaaaccgtgcaaaggctcctgcccgtcctgtgccaggctcacggtctgaccccggaacaagtcgtggccatcgctagccatgatggagggaaacaggccctggagactgtacagagactcctccctgtcctttgccaagcacacggacttacccctgaacaggtcgtggctattgcatcgaacgggggggggaaacaagcgctcgaaactgtgcaacgcctcctgccagtgttgtgccaggcacatggcttgactcccgagcaagtcgtcgccattgcatcaaacaacggaggaaaacaggcactcgagactgtgcaaagactgttgccggtgctatgccaggcacacggcttgaccccggagcaagtggtggctatcgcctcgcacgatggcggaaaacaagcactcgaaactgttcagcggctactgccggtgctctgccaggctcacggactgactccagaacaggtggtcgcaatcgcgtcgaacggtggaggacggcctgccctcgagagcattgtggcgcagctctcccgcccggatcctgctttggccgcgctgaccaacgaccacctggtggccctcgcctgtttggggggtcggcctgctttggacgccgtcaagaagggcctgcctcatgcccccgcactcatcaagcggaccaatagacgcatcccggaaaggacttctcaccgggtg

SEQ ID NO: 418 - protein, 798 aa, Homo sapiens
  SITE 1..798: note = DBD170, TAL08
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASRHGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRV

SEQ ID NO: 419 - DNA, 2394 bp, Homo sapiens
  misc_feature 1..2394: note = DBD170, TAL08
  source 1..2394: mol_type = genomic DNA
cacatggtggacctccggactcttggttactcccaacagcagcaggaaaagatcaagcctaaggtccgctccactgtggcccaacaccacgaggcccttgtgggacatggattcacccacgcccacatcgtggcgctgtcgcagcaccctgctgccttggggaccgtcgccgtgaagtaccaggacatgatcgccgccttacccgaagccacccacgaggcaatcgtgggagtgggtaaaaggggtgccggtgcaagagctctggaggcccttctcaccgtggccggagaacttagagggcctccactccagctggataccggtcagctgttgaagattgctaagagaggaggggtgaccgccgtggaagccgtgcatgcgtggcgcaacgctctgactggagccccactgaacttgacccccgaacaggtggtcgccatcgcctccaataacgggggcaaacaggcgctggaaaccgtgcagagattgctgcctgtcttatgccaagcacacggactcacccccgagcaagtcgtggctatcgcctccagacacggcgggaagcaagctctggagactgtgcagagactgctacccgtgttgtgtcaagcccacggtcttactccggagcaagtggtcgctattgcctcgcatgatggtggtaaacaggccctggagacagtgcagcgcttgctccctgtgctgtgtcaggcacacggcctgacaccggaacaggtggtggccatcgcttcgaacattggaggtaaacaggcattggagacagtccagaggctcctgccggtcctctgccaagctcacggtctgactcctgaacaagtagtcgccattgcatcccacgatggcggtaagcaggctcttgaaactgtccagagactgttacctgtgctctgtcaggcccatggactgacgcctgaacaggtcgtcgcgattgcgtcaaacatcggtggaaagcaagcgcttgaaacggtccagcgcctactgcctgtgttgtgccaagcgcacggcctcacaccagaacaagtcgtcgcaatcgcaagccatgatggcggaaagcaggcactagaaactgttcagcgccttctacccgtcttgtgccaggctcatggactcactcccgaacaagtggtggctattgcgtccaatggaggcggtaaacaagccctcgaaaccgtccagcggcttctgccggttctgtgccaggcacatggcttgactcctgagcaagttgtcgcgattgcctccaacattgggggcaagcaagcactcgaaacagtgcagaggcttctaccagtgctctgccaggcccacggtttgacccctgaacaggtggtcgccatcgcctccaacaacggcggaaaacaagcactggaaactgtgcaaagactgcttcctgtgctgtgtcaggcgcatggcctcaccccggaacaagtggtggccatagcgtcgcacgacggaggcaagcaggcccttgaaacggtgcagcgactgttgcccgtcctttgtcaggctcacgggttaaccccagaacaggtcgtggcaatcgcctctcatgacggggggaagcaggccttggagactgtccaaaggttgctgccagtgctgtgccaagcccacgggctcactccagagcaagtagtggcgatagcgagccacgacggcgggaaacaggctctggaaaccgtacagaggttgctccccgtgctttgtcaagcacatggtctgacccccgagcaagtcgtggccattgccagccatgacggagggaaacaggcacttgaaaccgtccaaaggctgctgcctgtgctttgccaggcgcatgggcttactcctgaacaagtggtggcgattgcttcaaatatcggtggcaaacaagctttggaaacagtccagcggttgctcccagtcctctgtcaagctcacgggctgacgccagaacaggtcgtggcaattgcgtcacacgacggaggaaaacaggcgttggaaaccgttcagcggctcttgcccgtgctatgccaggcccacggactgactccggaacaggtggttgccatcgccagcaacaacggagggaagcaggcgctcgagactgtacagcgacttctccccgtcctgtgtcaagcccatggcctgacgccggaacaggtggtagccattgcgagcaacggaggaggacgcccagccctagagagcatcgtggcccaactgtcgcggccggatccagctctggccgcattgaccaacgaccacctggtcgctctggcttgtctgggaggacgaccggccctggatgccgtgaagaagggcctgccacatgcccctgccctcattaagcggactaacagaaggatccccgagaggacctcacacagagtc

SEQ ID NO: 420 - protein, 798 aa, Homo sapiens
  SITE 1..798: note = DBD088, TAL04
HMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRV

SEQ ID NO: 421 - DNA, 2394 bp, Homo sapiens
  misc_feature 1..2394: note = DBD088, TAL04
  source 1..2394: mol_type = genomic DNA
cacatggtggacctgagaaccctgggatactcgcagcagcagcaggaaaagatcaagcccaaagtgcggtccactgtggcccagcaccatgaggccctcgtcggacacggattcacccacgcgcacatcgtggctctgtcacagcatcccgccgccttgggaaccgtggctgtgaagtaccaggacatgattgctgcacttcctgaggcgacccacgaagccattgtgggagtcggaaaacggggtgcaggcgctagagccctggaggccctgttaacggtggcaggcgaactgcggggaccaccattgcagctcgataccggccagctgctgaagattgccaagaggggcggagtcacagcggtggaggcggtccatgcctggcgcaacgctcttactggggctccactgaacctgacccctgaacaggtagtggccatcgcatcgaacaatggcggtaagcaggctctcgaaaccgtccaaaggctccttccggtgttatgccaggctcatgggctgacgcctgaacaagtggtcgcaatcgcatcgcatgacggggggaaacaggcattagaaaccgtgcaacgcctccttcctgtcctttgtcaagcacatggattgactcctgagcaagttgtcgcgattgcctccaacattggcggcaaacaggcactggaaaccgttcaacggctcctccccgtactgtgccaggcacacggcctgacccccgaacaagtcgtcgccattgcctcgaacatcggaggcaagcaggcgcttgaaaccgtgcagcgcttgctgcctgtgctctgccaagcgcacggcctaacccctgaacaggtggtagcaattgcttcccacgacggtgggaagcaggccctcgagacagtgcaaagactgttgccggtcctctgccaggcccacggacttacccccgagcaagtcgtggctattgcgtccaataatggcggaaaacaggcacttgagacagtccagaggctgctgcctgtcttatgccaagcccacggtcttactccagaacaggtggtcgctatcgcatcaaacattggaggaaaacaggcgctggaaactgtccaacgcctgttgcccgtgctctgtcaggctcacgggctgactcccgagcaagtggtggcaatcgcctccaacgggggaggaaagcaggccttggaaaccgtacagcggctgttgcctgtgttgtgccaagctcacggtctgacgccagagcaagtggtggccattgcttcgcacgacggcgggaagcaggcactcgaaacggtgcagaggttgcttcccgtcttgtgccaggcgcacggactcaccccagaacaagtggtcgcaattgctagcaacattggtggcaaacaagcgctcgaaactgtacaacggctgctcccggtgctttgccaagcccacgggcttaccccagagcaagtggtggccattgcgagcaacatcgggggcaagcaagcgcttgagactgtacaaagactgctgcccgtgctgtgccaagcccacggcttaaccccggaacaggtcgtggccatcgcctcgaatggaggtggaaagcaagccctggagactgtgcagcggttgctgcccgttctgtgtcaagctcacggactgacaccggaacaggtggtggctatcgcaagcaacaacggcggaaagcaagccctggagactgtgcagcggctgctgccagtgctgtgtcaggcgcatggactgacccccgaacaagtcgtagccatagccagccgccacggagggaagcaggccctggaaacagtccagcgcctcttgcccgtgttgtgtcaagcgcatggcctcacccctgaacaggtcgttgccattgcgtccagacatggagggaagcaagcactagaaacagtgcagagactgctacccgtcctgtgccaggcccatggactcacgccggagcaagtggtggcgatagcatcgaacattggtgggaaacaagcccttgaaactgtccagagactgttacctgtgctttgtcaggcacatgggctcacaccagaacaggtggtcgccatcgctagcaacggagggggaaaacaggctttggagactgtccagcggctcctaccggtgctctgccaagcacacggattgacgcccgaacaggttgtggcgattgcgtccaacggtggaggacggcctgccctggagagcattgtggctcagctctccagacctgacccagccctcgcagctctgactaacgatcatctggtggcgctggcctgtttggggggacgccctgctttggacgccgtgaagaagggtctgccacatgcccccgcactgatcaagcgcaccaaccggcgaatccctgagcgcacttcccatcgagtg

SEQ ID NO: 422 - DNA, 21 bp, synthetic construct
  misc_feature 1..21: note = Synthetic: DBD386, ZF24
  source 1..21: mol_type = other DNA
agccatgacactgcactaccc

SEQ ID NO: 423 - DNA, 21 bp, synthetic construct
  misc_feature 1..21: note = Synthetic: DBD374, ZF16
  source 1..21: mol_type = other DNA
attctgcaccagctgagggga

SEQ ID NO: 424 - DNA, 21 bp, synthetic construct
  misc_feature 1..21: note = Synthetic: DBD360, ZF07
  source 1..21: mol_type = other DNA
gatccaaatgctaatgtaacc

SEQ ID NO: 425 - DNA, 21 bp, synthetic construct
  misc_feature 1..21: note = Synthetic: DBD358, ZF05
  source 1..21: mol_type = other DNA
gcatgacaagttcctaaacga

SEQ ID NO: 426 - DNA, 18 bp, Mus musculus
  misc_feature 1..18: note = DBD455, TAL37
  source 1..18: mol_type = genomic DNA
gatatctaagtcaggttc

SEQ ID NO: 427 - DNA, 18 bp, Mus musculus
  misc_feature 1..18: note = DBD452, TAL34
  source 1..18: mol_type = genomic DNA
atcccttactataaactc

SEQ ID NO: 428 - DNA, 18 bp, Mus musculus
  misc_feature 1..18: note = DBD429, TAL27
  source 1..18: mol_type = genomic DNA
aagatagatgaaggtcac

SEQ ID NO: 429 - DNA, 18 bp, Homo sapiens
  misc_feature 1..18: note = DBD330, TAL14
  source 1..18: mol_type = genomic DNA
gaagggcatggctatagc

SEQ ID NO: 430 - DNA, 18 bp, Homo sapiens
  misc_feature 1..18: note = DBD203, TAL09
  source 1..18: mol_type = genomic DNA
gacttagcaaaacctgct

SEQ ID NO: 431 - DNA, 18 bp, Homo sapiens
  misc_feature 1..18: note = DBD170, TAL08
  source 1..18: mol_type = genomic DNA
ggcacactagccccacgt

SEQ ID NO: 432 - DNA, 18 bp, Homo sapiens
  misc_feature 1..18: note = DBD088, TAL04
  source 1..18: mol_type = genomic DNA
gcaacgatcaatgggatt

SEQ ID NO: 433 - DNA, 1089 bp, synthetic construct
  misc_feature 1..1089: note = Synthetic: OEC695, ZF07-KRAB
  source 1..1089: mol_type = other DNA
atggcccccaagaagaagcggaaggtgggcatccacggcgtgcccgccgccggcagcagcggatccctcgagcctggcgaaaagccttataagtgcccagaatgcggaaagagcttctccgacaagaaggatctgacccgccaccaaaggacccacactggggagaagccttacaagtgtccggagtgcggaaagtccttttcgcaatcgtccagcctggtccggcaccagaggacgcacaccggagagaagccatacaaatgcccggagtgtgggaagtcgttctccaccactggaaacctcactgtgcaccagcgcacgcatactggggaaaagccctataagtgtccagagtgcgggaagtccttctcaacctccggcgaattggtcagacaccagcggacacacaccggcgagaagccctacaagtgccctgagtgcggcaaaagcttcagcactaccggcaacctgactgtgcatcagcggacccataccggagaaaaaccctacaaatgtcccgaatgcggcaaatccttctccacctcccactcgctgaccgaacaccagagaacccatactggagaaaagccgtacaagtgcccggaatgtggaaagtctttcagcacctccggaaaccttgtgcggcatcagcgcactcacaccggcaaaaagacctcagcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctga

SEQ ID NO: 434 - DNA, 2871 bp, synthetic construct
  misc_feature 1..2871: note = Synthetic: OEC693, TAL27-KRAB
  source 1..2871: mol_type = other DNA
atggcccccaagaagaagcggaaggtgggcatccacggcgtgcccgccgccggcagcagcggatcccacatggtggacctccgcacgctgggatactcgcaacagcagcaggagaagattaagcctaaggtccggtccaccgtggctcagcaccatgaggcgctagtgggccacggattcacccacgcgcatattgtcgccctctcgcaacatcccgccgcactgggaaccgtcgccgtgaagtaccaggacatgattgccgccctgcctgaagctacccatgaagccattgtgggcgtgggaaagagaggagcaggggccagagccttggaggccctgctcacggtagccggagagctcagaggcccaccactgcagcttgacaccggccaacttctgaagatcgccaagcggggaggagtgaccgccgtggaggcagtgcatgcctggcgcaacgccttgactggagccccgctgaacctgactccggagcaagtggtggctatcgcgtccaacatcggaggaaaacaggccctggaaacggtgcaaagactgctgccggtcctctgtcaagcgcacggactcactcccgagcaagttgtggctatcgcttccaacatcggggggaaacaggctctggaaactgtccaaaggctgttacctgtgctgtgccaagcacacggactgaccccagagcaagtggtcgccatagcctccaacaacggggggaagcaagccctggagactgtgcagcggttgctccccgtcctgtgccaggcccacggattgactcccgaacaagtggtggccattgcgtcaaacatcgggggcaagcaagcgttggaaacagtccaacggttgcttcccgtcctttgccaggcccacggtctgaccccggagcaagtcgtcgccattgctagtaacgggggaggaaagcaagctcttgaaaccgtccagcggcttcttccggtgctctgccaagcacacggcctcacacccgagcaagtagtggccattgcctcaaacattgggggaaagcaggcattggaaactgtgcaacggctgcttcctgtcttgtgccaggcgcatgggctgactccagaacaggtggtcgctatcgcgagtaacaacggtggcaaacaggcactggaaaccgtgcagaggctcttgcctgtcctgtgtcaggcacacggactgacgccagaacaggtggtcgctattgcgagcaatatcggcggaaagcaggcgttagaaactgtgcagagactgctcccagtgctatgccaggctcatgggttgactcctgaacaagtggtggctatcgcctcaaacggtggcggtaaacaggcgcttgagacagtgcaaaggctccttcccgtgttgtgccaagcgcatggcctgacccctgaacaggtcgtggctatcgcgtcaaacaacggaggcaagcaggccctagaaacggtacaacggctgctgcccgtactgtgccaggcccacggactgaccccagaacaggtggtcgcaattgcctccaacattggcgggaagcaggcgctagaaaccgtgcagcggcttctgcccgtcctctgccaggcccacggtctgactcctgagcaagtcgtcgcaatcgccagcaacatcggcggaaaacaagccctcgaaaccgtccagcgactgctgcctgtcctatgccaggcccacgggttgaccccggaacaggtcgtcgccattgcttccaataatggaggcaaacaggcactcgaaacggtgcagagacttctgccggtgctgtgtcaagctcacggcttgacccccgagcaagtggtggcaatcgcttcacgacacggtggaaaacaggccctggagacagtccagagactgctccccgtgctttgccaagctcacggacttactccggaacaagtcgtggcgattgcgtccaacggtggtggaaagcaggccttggaaacggtccagagactgttgccggtcttgtgccaggcgcatggtctgacccccgaacaagtcgtggcaattgcatcccatgacggcgggaaacaggctttggagactgtccagcgcctgttgccagtcctttgtcaagcccacgggttgacacccgaacaggtcgtcgcaatcgcctcgaacatcggtggcaagcaggcccttgaaactgtccaacgcctcctcccggtgttgtgtcaggctcacggcctgactccagaacaagtcgtggccatagcgtcacacgacggtggacggccagccctggaatccatcgtggcgcagctgtccagacccgacccagcactggcggccctcactaacgatcaccttgtggccctggcctgtctgggaggtagaccagccctcgacgctgtgaagaagggactgccgcatgcaccagccctgatcaagcgaaccaaccggcgcattcccgagagaacttcacacagagtggctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctga

SEQ ID NO: 435 - DNA, 1088 bp, synthetic construct
  misc_feature 1..1088: note = Synthetic: OEC151, ZF24-KRAB
  source 1..1088: mol_type = other DNA
atggcccccaagaagaagcggaaggtgggcatccacggcgtgcccgccgccggcagcagcggatccctggaacccggagagaagccctataagtgtccggaatgcggaaagagtttcagctcgaagaagcacctcgccgagcaccaacggacccacacgggagaaaagccctacaagtgcccagagtgcggaaagtcgttcagccaaaactccaccctgaccgaacaccagcggactcataccggggagaaaccttacaagtgtccagaatgcgggaagtcgttctcccaatccggagacttgcgcagacaccagcgcactcacaccggcgaaaaaccgtacaaatgcccggagtgtggaaagtcattttccacccacctggatctgatcaggcatcagcgcacccacactggcgaaaagccttacaaatgtccggagtgcggcaaatccttctctgaccccggcaacctcgtgcgccatcagaggactcacactggggagaagccttataagtgccccgaatgtggaaagagcttctccacctccggcaaccttaccgagcatcagcggacacacaccggggaaaagccgtacaagtgccctgagtgtggcaaatcattctccgaacggagccatctgcgggagcaccagagaacccacaccggaaagaaaacttccgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctg

SEQ ID NO: 436 - DNA, 1088 bp, synthetic construct
  misc_feature 1..1088: note = Synthetic: OEC144, ZF16-KRAB
  source 1..1088: mol_type = other DNA
atggcccccaagaagaagcggaaggtgggcatccacggcgtgcccgccgccggcagcagcggatccctggagccgggagagaagccttataagtgtcctgagtgcggcaaatcattctcccaacgcgcccacctcgagagacaccagagaacccacaccggggaaaagccgtacaagtgccctgagtgtgggaagtccttttcccggtccgaccatttgaccaaccatcagcggacccacactggggagaagccatacaaatgcccagaatgcggcaaaagcttctcccggaacgacgcacttaccgaacatcagcgcacgcacaccggagagaaaccctataagtgccccgaatgtggaaagtcattctcccgggccgacaacctgacagagcaccagcggactcacactggagaaaagccttacaagtgtcccgaatgcgggaagtcgttcagctcgaagaaggctctgaccgaacaccagaggacccataccggcgaaaagccctataaatgcccggagtgcggaaagagcttcagcaggaacgatgccctcaccgagcaccaacgcactcatactggcgaaaaaccgtacaaatgtccggaatgcggaaagtccttctcgcacaagaacgcgctgcagaatcaccagcgcactcacaccggcaaaaagacctctgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctg

SEQ ID NO: 437 - DNA, 1970 bp, synthetic construct
  misc_feature 1..1970: note = Synthetic: OEC134, ZF07-MQ1
  source 1..1970: mol_type = other DNA
atggcccccaagaagaagcggaaggtgggcatccacggcgtgcccgccgccggcagcagcggatccctcgagcctggcgaaaagccttataagtgcccagaatgcggaaagagcttctccgacaagaaggatctgacccgccaccaaaggacccacactggggagaagccttacaagtgtccggagtgcggaaagtccttttcgcaatcgtccagcctggtccggcaccagaggacgcacaccggagagaagccatacaaatgcccggagtgtgggaagtcgttctccaccactggaaacctcactgtgcaccagcgcacgcatactggggaaaagccctataagtgtccagagtgcgggaagtccttctcaacctccggcgaattggtcagacaccagcggacacacaccggcgagaagccctacaagtgccctgagtgcggcaaaagcttcagcactaccggcaacctgactgtgcatcagcggacccataccggagaaaaaccctacaaatgtcccgaatgcggcaaatccttctccacctcccactcgctgaccgaacaccagagaacccatactggagaaaagccgtacaagtgcccggaatgtggaaagtctttcagcacctccggaaaccttgtgcggcatcagcgcactcacaccggcaaaaagacctcagcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctg

SEQ ID NO: 438 - DNA, 1970 bp, synthetic construct
  misc_feature 1..1970: note = Synthetic: OEC129, ZF05-MQ1
  source 1..1970: mol_type = other DNA
atggcccccaagaagaagcggaaggtgggcatccacggcgtgcccgccgccggcagcagcggatccctggagcccggagagaaaccgtataagtgcccggaatgtggaaagtccttctcccaatccggccacctgactgagcatcagcgcacccacactggcgaaaagccgtacaagtgccctgagtgcgggaagtcgtttagccagcgggcgaacttgcgcgctcatcagcggactcataccggggagaagccctataaatgcccggagtgcggaaagagcttcagcaccaagaactccctgactgaacaccagcggacccacaccggagagaagccttacaagtgtcctgaatgcgggaaatccttctccacctccggttcactcgtgcgccaccagagaacgcacaccggtgaaaagccttataagtgtccagagtgcggcaaaagcttcagccagtccggcaacctcaccgagcaccaacggactcacactggagaaaagccctacaaatgtccggaatgcggcaaatcattctcgcaagccggtcaccttgcctcgcaccagcgcacacacaccggggaaaagccatacaaatgccccgaatgcggaaagtcgttctctcaatccggcgacctgagaaggcatcagaggacccataccggaaagaaaacctccgcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctg

SEQ ID NO: 439 - DNA, 1088 bp, synthetic construct
  misc_feature 1..1088: note = Synthetic: OEC126, ZF05-KRAB
  source 1..1088: mol_type = other DNA
atggcccccaagaagaagcggaaggtgggcatccacggcgtgcccgccgccggcagcagcggatccctggagcccggagagaaaccgtataagtgcccggaatgtggaaagtccttctcccaatccggccacctgactgagcatcagcgcacccacactggcgaaaagccgtacaagtgccctgagtgcgggaagtcgtttagccagcgggcgaacttgcgcgctcatcagcggactcataccggggagaagccctataaatgcccggagtgcggaaagagcttcagcaccaagaactccctgactgaacaccagcggacccacaccggagagaagccttacaagtgtcctgaatgcgggaaatccttctccacctccggttcactcgtgcgccaccagagaacgcacaccggtgaaaagccttataagtgtccagagtgcggcaaaagcttcagccagtccggcaacctcaccgagcaccaacggactcacactggagaaaagccctacaaatgtccggaatgcggcaaatcattctcgcaagccggtcaccttgcctcgcaccagcgcacacacaccggggaaaagccatacaaatgccccgaatgcggaaagtcgttctctcaatccggcgacctgagaaggcatcagaggacccataccggaaagaaaacctccgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctg

SEQ ID NO: 440 - DNA, 2870 bp, synthetic construct
  misc_feature 1..2870: note = Synthetic: OEC103, TAL14-KRAB
  source 1..2870: mol_type = other DNA
atggcccccaagaagaagcggaaggtgggcatccacggcgtgcccgccgccggcagcagcggatcccacatggtcgatcttcgcaccctcgggtactcgcagcagcaacaggaaaagatcaagccgaaagtcaggtccaccgtcgcccagcaccacgaggcactggtcggccacggattcacccacgcccacatcgtcgcgctgtctcaacatccagccgccctgggaactgtcgccgtcaagtaccaagatatgatcgcggcactgcctgaagcaacccatgaggccatcgtgggtgtgggaaagagaggagcaggggctagagccctggaagcgctcctgaccgtggcgggagagcttagaggccctcccctgcaattggacaccggccaacttctgaagattgcaaaaaggggtggagtgactgccgtggaagcagtgcacgcttggcggaacgcactgactggagcacccctgaatttgacccccgagcaagtggttgccatcgcaagcaacatcggagggaaacaagcactggaaactgtgcagcgcctgcttccggtcctgtgccaagctcacggactgactcctgaacaggttgtggccatcgcgtcgcatgacgggggaaaacaggcgctcgaaaccgtccaaagactgctcccggtgctgtgtcaggctcacggtttgaccccagaacaagttgtcgctatcgcctcacacgacggcgggaagcaagctctggagactgtgcaaagactccttcctgtgctgtgccaagctcatggattgaccccggaacaggtcgtcgccatcgcttccaatggcggaggtaaacaggccttggaaacggtgcagaggctgctgccagtcttgtgtcaggcgcacgggctgactccagagcaagtagtagccatcgccagcaatggaggcggtaaacaagcgctcgagaccgtccagcgcttgttgccagtgttgtgccaggcccatgggctgacgcccgaacaggtggtggccattgcatccaacattggggggaaacaggcgttggagacagtccaacggcttctgcccgtcctttgtcaggcacacggactcactcctgaacaagtcgttgccattgcctcgaatatcgggggtaagcaagccctcgagactgtgcagagattgttgcctgtcctgtgtcaagcgcatggtctgactcccgaacaggtggtggcgattgcttcgaacaatggaggaaaacaagcattggaaaccgtccagaggctgttaccagtcctttgccaggcacatggactgaccccggaacaggtcgtcgcaatcgcatcgaacattggaggcaagcaggctctcgaaacggtccagagactgctgcctgtgctctgccaagcccacggcctcactccggagcaagtcgtggcaatcgcctcaaacatcggtggaaagcaggcccttgagactgtccagcgactgctcccagtgctctgccaggcgcatggactcacacccgagcaggtggtcgccattgctagcaacaacggaggcaaacaagccctggagacagtgcaacgcttgctgcccgtgctttgtcaagcgcacggacttactccggaacaagtagtggctattgcgagtaggcacggcggtaagcaagccctggagacagtgcagcggcttttgccggtgctctgtcaggcccacgggctcacaccggaacaggtcgtagctattgcgtcccatgacggcggaaagcaagccttagaaactgtgcagcggttgctgcctgttctgtgccaggctcatgggctcactcccgagcaagtggtcgcgattgcgagcaacggaggtggcaagcaagcactagaaaccgtgcaacgactgctgccggtcttgtgccaagcgcatggcctgacacccgaacaggtggtggctatcgcgtccaacaacgggggcaaacaggccctggaaacagtccaaaggctgttgccggtcctctgtcaagctcacggactaacccccgaacaggtggtggcaatagcttcgcggcatgggggcaagcaggcactcgaaacagtgcagaggttgctcccggtactttgccaagcacatggcttgacccctgagcaagtcgtggccatcgcatccaacggcggagggaagcaagcgcttgaaactgtccaacgcctcctgcccgtgttgtgtcaagcccatggccttacgcctgaacaagtcgtcgctattgcaagtaacggaggagggaggcctgccctggagtccattgtcgcccaactgtcgcgacccgatccagccctggcagccctcactaacgaccatctcgtggcgctggcatgcctcggtggtagaccagctctggacgccgtgaagaagggacttcctcatgcccccgcactgatcaagcgcactaaccggaggatccctgaaagaacctcccacagagtggcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctg

SEQ ID NO: 441 - DNA, 2870 bp, synthetic construct
  misc_feature 1..2870: note = Synthetic: OEC099, TAL09-KRAB
  source 1..2870: mol_type = other DNA
atggcccccaagaagaagcggaaggtgggcatccacggcgtgcccgccgccggcagcagcggatcccacatggtggacctccggaccctgggatactcccagcagcagcaggaaaagatcaagccgaaggtccgctccactgtggctcaacaccacgaagcccttgtgggacacggcttcactcatgcacacattgtggcattgtcccaacaccctgccgctctgggaactgtggcagtgaagtaccaggacatgatagccgcactgcccgaagcaacacacgaggccatcgtcggtgtgggaaaaagaggagcgggagccagagcactcgaagcgctcctgacagtggcgggagaactccgcggaccacctctgcagctggacactggccagctgctgaagattgcaaaacgcggtggagtcaccgctgtggaggcggtgcatgcctggagaaatgccctgactggtgcccccttgaacctgacccccgagcaagtcgtcgcaattgcttcgaacaacggtggtaaacaggctctcgaaaccgtccaacggctgctcccagtcctatgccaagcccatggacttacgcccgaacaagtggttgctatagcctccaatatcggtgggaagcaggctctggagacagtccaaagactcctgccggtcctctgccaagcgcacggactcaccccagagcaagtagtggccattgcctcccacgatggtggcaagcaggcccttgaaacagtccagagactgttacccgtgctttgtcaagcccacgggctcactcccgaacaggtggtggcgattgcctctaacggcggcggcaaacaggcgctcgagactgtccagcggcttctaccggtgttgtgccaggcccatggtctgacacccgaacaggtggttgcaattgcctccaacggaggcgggaagcaagccctcgaaacggtccagcggttgttgccggtcctgtgtcaggcgcacgggttgaccccagaacaggtcgtcgctattgcctcaaacatcggtggtaagcaggcgctggaaactgtgcagcgactcctgcctgtgctctgtcaagcgcacgggcttacgccagaacaagtggtcgctatcgcgtccaacaacggcggcaagcaagctctggaaaccgtccaaaggctcttgcccgtcctatgtcaggcccacggcctcacccctgaacaggttgtcgccatcgccagccacgacggcggtaagcaagccttggaaaccgtgcaacgactgcttcccgtgttgtgtcaggctcatggcctcactcctgagcaagttgtggccatcgcttcgaatattgggggcaaacaagccctggaaacggtgcagcggttgctgcctgtcttgtgtcaagctcacgggctgactcctgaacaagtcgtggccatagcgtccaacattgggggaaagcaggcgttggaaactgtccagaggctgttgcccgtgctctgccaggctcacggcctgacccctgaacaggtggtcgcaatcgcctccaacatcggagggaagcaggcactggaaacagtgcagcggctgctgcccgtgctgtgccaagctcatggactcactccggaacaggtcgttgcaatcgcatcaaacattggaggaaagcaggccctcgagacagtgcagagactgctccctgtgctttgccaggcgcatggccttaccccggaacaagtggtagccatcgcgtcgcatgacggcggaaagcaggctttggaaaccgtgcaaaggctcctgcccgtcctgtgccaggctcacggtctgaccccggaacaagtcgtggccatcgctagccatgatggagggaaacaggccctggagactgtacagagactcctccctgtcctttgccaagcacacggacttacccctgaacaggtcgtggctattgcatcgaacgggggggggaaacaagcgctcgaaactgtgcaacgcctcctgccagtgttgtgccaggcacatggcttgactcccgagcaagtcgtcgccattgcatcaaacaacggaggaaaacaggcactcgagactgtgcaaagactgttgccggtgctatgccaggcacacggcttgaccccggagcaagtggtggctatcgcctcgcacgatggcggaaaacaagcactcgaaactgttcagcggctactgccggtgctctgccaggctcacggactgactccagaacaggtggtcgcaatcgcgtcgaacggtggaggacggcctgccctcgagagcattgtggcgcagctctcccgcccggatcctgctttggccgcgctgaccaacgaccacctggtggccctcgcctgtttggggggtcggcctgctttggacgccgtcaagaagggcctgcctcatgcccccgcactcatcaagcggaccaatagacgcatcccggaaaggacttctcaccgggtggcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctg

SEQ ID NO: 442 - DNA, 2870 bp, synthetic construct
  misc_feature 1..2870: note = Synthetic: OEC095, TAL08-KRAB
  source 1..2870: mol_type = other DNA
atggcccccaagaagaagcggaaggtgggcatccacggcgtgcccgccgccggcagcagcggatcccacatggtggacctccggactcttggttactcccaacagcagcaggaaaagatcaagcctaaggtccgctccactgtggcccaacaccacgaggcccttgtgggacatggattcacccacgcccacatcgtggcgctgtcgcagcaccctgctgccttggggaccgtcgccgtgaagtaccaggacatgatcgccgccttacccgaagccacccacgaggcaatcgtgggagtgggtaaaaggggtgccggtgcaagagctctggaggcccttctcaccgtggccggagaacttagagggcctccactccagctggataccggtcagctgttgaagattgctaagagaggaggggtgaccgccgtggaagccgtgcatgcgtggcgcaacgctctgactggagccccactgaacttgacccccgaacaggtggtcgccatcgcctccaataacgggggcaaacaggcgctggaaaccgtgcagagattgctgcctgtcttatgccaagcacacggactcacccccgagcaagtcgtggctatcgcctccagacacggcgggaagcaagctctggagactgtgcagagactgctacccgtgttgtgtcaagcccacggtcttactccggagcaagtggtcgctattgcctcgcatgatggtggtaaacaggccctggagacagtgcagcgcttgctccctgtgctgtgtcaggcacacggcctgacaccggaacaggtggtggccatcgcttcgaacattggaggtaaacaggcattggagacagtccagaggctcctgccggtcctctgccaagctcacggtctgactcctgaacaagtagtcgccattgcatcccacgatggcggtaagcaggctcttgaaactgtccagagactgttacctgtgctctgtcaggcccatggactgacgcctgaacaggtcgtcgcgattgcgtcaaacatcggtggaaagcaagcgcttgaaacggtccagcgcctactgcctgtgttgtgccaagcgcacggcctcacaccagaacaagtcgtcgcaatcgcaagccatgatggcggaaagcaggcactagaaactgttcagcgccttctacccgtcttgtgccaggctcatggactcactcccgaacaagtggtggctattgcgtccaatggaggcggtaaacaagccctcgaaaccgtccagcggcttctgccggttctgtgccaggcacatggcttgactcctgagcaagttgtcgcgattgcctccaacattgggggcaagcaagcactcgaaacagtgcagaggcttctaccagtgctctgccaggcccacggtttgacccctgaacaggtggtcgccatcgcctccaacaacggcggaaaacaagcactggaaactgtgcaaagactgcttcctgtgctgtgtcaggcgcatggcctcaccccggaacaagtggtggccatagcgtcgcacgacggaggcaagcaggcccttgaaacggtgcagcgactgttgcccgtcctttgtcaggctcacgggttaaccccagaacaggtcgtggcaatcgcctctcatgacggggggaagcaggccttggagactgtccaaaggttgctgccagtgctgtgccaagcccacgggctcactccagagcaagtagtggcgatagcgagccacgacggcgggaaacaggctctggaaaccgtacagaggttgctccccgtgctttgtcaagcacatggtctgacccccgagcaagtcgtggccattgccagccatgacggagggaaacaggcacttgaaaccgtccaaaggctgctgcctgtgctttgccaggcgcatgggcttactcctgaacaagtggtggcgattgcttcaaatatcggtggcaaacaagctttggaaacagtccagcggttgctcccagtcctctgtcaagctcacgggctgacgccagaacaggtcgtggcaattgcgtcacacgacggaggaaaacaggcgttggaaaccgttcagcggctcttgcccgtgctatgccaggcccacggactgactccggaacaggtggttgccatcgccagcaacaacggagggaagcaggcgctcgagactgtacagcgacttctccccgtcctgtgtcaagcccatggcctgacgccggaacaggtggtagccattgcgagcaacggaggaggacgcccagccctagagagcatcgtggcccaactgtcgcggccggatccagctctggccgcattgaccaacgaccacctggtcgctctggcttgtctgggaggacgaccggccctggatgccgtgaagaagggcctgccacatgcccctgccctcattaagcggactaacagaaggatccccgagaggacctcacacagagtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctg

SEQ ID NO: 443 - DNA, 2870 bp, synthetic construct
  misc_feature 1..2870: note = Synthetic: OEC087, TAL04-KRAB
  source 1..2870: mol_type = other DNA
atggcccccaagaagaagcggaaggtgggcatccacggcgtgcccgccgccggcagcagcggatcccacatggtggacctgagaaccctgggatactcgcagcagcagcaggaaaagatcaagcccaaagtgcggtccactgtggcccagcaccatgaggccctcgtcggacacggattcacccacgcgcacatcgtggctctgtcacagcatcccgccgccttgggaaccgtggctgtgaagtaccaggacatgattgctgcacttcctgaggcgacccacgaagccattgtgggagtcggaaaacggggtgcaggcgctagagccctggaggccctgttaacggtggcaggcgaactgcggggaccaccattgcagctcgataccggccagctgctgaagattgccaagaggggcggagtcacagcggtggaggcggtccatgcctggcgcaacgctcttactggggctccactgaacctgacccctgaacaggtagtggccatcgcatcgaacaatggcggtaagcaggctctcgaaaccgtccaaaggctccttccggtgttatgccaggctcatgggctgacgcctgaacaagtggtcgcaatcgcatcgcatgacggggggaaacaggcattagaaaccgtgcaacgcctccttcctgtcctttgtcaagcacatggattgactcctgagcaagttgtcgcgattgcctccaacattggcggcaaacaggcactggaaaccgttcaacggctcctccccgtactgtgccaggcacacggcctgacccccgaacaagtcgtcgccattgcctcgaacatcggaggcaagcaggcgcttgaaaccgtgcagcgcttgctgcctgtgctctgccaagcgcacggcctaacccctgaacaggtggtagcaattgcttcccacgacggtgggaagcaggccctcgagacagtgcaaagactgttgccggtcctctgccaggcccacggacttacccccgagcaagtcgtggctattgcgtccaataatggcggaaaacaggcacttgagacagtccagaggctgctgcctgtcttatgccaagcccacggtcttactccagaacaggtggtcgctatcgcatcaaacattggaggaaaacaggcgctggaaactgtccaacgcctgttgcccgtgctctgtcaggctcacgggctgactcccgagcaagtggtggcaatcgcctccaacgggggaggaaagcaggccttggaaaccgtacagcggctgttgcctgtgttgtgccaagctcacggtctgacgccagagcaagtggtggccattgcttcgcacgacggcgggaagcaggcactcgaaacggtgcagaggttgcttcccgtcttgtgccaggcgcacggactcaccccagaacaagtggtcgcaattgctagcaacattggtggcaaacaagcgctcgaaactgtacaacggctgctcccggtgctttgccaagcccacgggcttaccccagagcaagtggtggccattgcgagcaacatcgggggcaagcaagcgcttgagactgtacaaagactgctgcccgtgctgtgccaagcccacggcttaaccccggaacaggtcgtggccatcgcctcgaatggaggtggaaagcaagccctggagactgtgcagcggttgctgcccgttctgtgtcaagctcacggactgacaccggaacaggtggtggctatcgcaagcaacaacggcggaaagcaagccctggagactgtgcagcggctgctgccagtgctgtgtcaggcgcatggactgacccccgaacaagtcgtagccatagccagccgccacggagggaagcaggccctggaaacagtccagcgcctcttgcccgtgttgtgtcaagcgcatggcctcacccctgaacaggtcgttgccattgcgtccagacatggagggaagcaagcactagaaacagtgcagagactgctacccgtcctgtgccaggcccatggactcacgccggagcaagtggtggcgatagcatcgaacattggtgggaaacaagcccttgaaactgtccagagactgttacctgtgctttgtcaggcacatgggctcacaccagaacaggtggtcgccatcgctagcaacggagggggaaaacaggctttggagactgtccagcggctcctaccggtgctctgccaagcacacggattgacgcccgaacaggttgtggcgattgcgtccaacggtggaggacggcctgccctggagagcattgtggctcagctctccagacctgacccagccctcgcagctctgactaacgatcatctggtggcgctggcctgtttggggggacgccctgctttggacgccgtgaagaagggtctgccacatgcccccgcactgatcaagcgcaccaaccggcgaatccctgagcgcacttcccatcgagtggcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctg

SEQ ID NO: 444 - DNA, 2850 bp, synthetic construct
  misc_feature 1..2850: note = Synthetic: OEC056, TAL37-KRAB
  source 1..2850: mol_type = other DNA
atggccccaaagaagaagcggaaagtcggaggttcaggcggctcgcacatggtggacctgagaactctgggttactcccagcaacagcaagaaaaaatcaagccaaaagtgcgcagcactgtcgcgcagcaccacgaggcccttgtggggcacgggttcacccacgctcacatcgtggccctgagccagcatccggcagccctgggaaccgtcgcagtgaagtaccaagacatgatcgccgccttgccggaagccactcacgaagcaattgtgggagtggggaagagaggagccggcgcaagagccctcgaagcgcttctgactgtggccggagaattgagaggccccccactgcagctggataccggacaactgctgaagatcgccaaacggggtggagtgaccgccgtggaagcggtgcacgcgtggagaaacgcgctgactggagccccgctcaacctgacccctgaacaggtcgtggctattgcctcaaacaacggcgggaagcaagcgttggagactgtacagcgcctgctgccagtgttgtgtcaagcccacgggctgactcccgagcaagtggtggccatagccagcaatatcggaggtaaacaggctctcgagactgtccaacgcctgctcccagtgctttgtcaggctcacggcctcactcctgaacaggtcgtagccattgccagcaacggtggcggcaaacaagccttagagactgtgcaacggctcttgccggtgctctgtcaggcccacggactgaccccagaacaagtagtcgctatcgcgtcaaacatcggcggcaagcaggctttggaaaccgtccagcggcttctgccggtgctgtgccaggcgcatgggctcacccccgaacaggtggtcgcaatcgcctcgaacggcggtggtaaacaagccctcgaaactgtccagcgcctcttgcccgtcctgtgtcaggcccatggcctgactccagaacaggtggtggcaatagcgtcccatgacgggggaaagcaagcccttgagactgtgcaacggttgctccccgtgttatgtcaggcacatggtctgacccccgaacaggtggtcgcaattgcgtcgaacggcggaggaaagcaagctctggaaacggtccaacggctgctccctgtgctgtgccaggcgcacggacttaccccagaacaagtggtcgccattgcgtccaacatcggagggaagcaggcgcttgaaaccgtgcagaggttgctgccggtgttatgccaagcacatggactaaccccggagcaagtggtggccattgcctcgaatattggggggaaacaagcgttagaaactgtgcagaggctgctgcctgtgctctgccaagcccatggtcttaccccggaacaggttgtggcgattgcatccaacaacggtggaaaacaggcccttgaaacggtgcagcggctccttcctgtcctgtgccaagcccatggactcacccctgaacaggtcgtggctattgcctccaacggaggcggcaagcaggccctggagacagtccagagactgctgcccgtgctgtgtcaagctcacggtctgactccggaacaggtggttgctatcgcttcgcacgacggcggaaaacaagctcttgaaaccgtgcaaaggctgctcccggtgctttgccaagctcatggcttgaccccagagcaagtcgtggcaatcgcgtccaatatcggtgggaagcaggctcttgagacagtgcaaagactcctccccgtcctctgtcaagcgcacgggctcactcctgaacaggtcgtcgctattgcttcgaacaacgggggcaaacaggcactagagactgtccaaagacttttacctgtcctctgccaggcacacggattgactcccgaacaagtcgtcgcgattgcctcaagacacgggggcaagcaagcgctggagactgttcagcgactgcttcctgtgctttgccaggcccacggtttgacccctgagcaagttgtggccatcgcctctaacggaggtggaaagcaggccttggaaacagtgcagagactgttgcccgtcttgtgccaggctcatgggcttacgcctgaacaagtcgtcgccatcgcttcgaacggaggagggaaacaggcgctggaaactgtgcagcgactccttccggtcctttgtcaagcacacggccttacgccggaacaagtggttgccatcgcaagccacgacggaggacgccctgccctcgagtccattgtggcccagctgtcacgcccagatcctgcactggccgctcttaccaacgatcacctggtcgctctcgcgtgcctgggaggaaggcctgccctggatgccgtcaagaagggactgcctcatgcccctgctttgattaagaggactaatcggcgcatccccgaacggaccagccacagggtggcatctgggagcggaggaggatccggcggagacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcgcccagcggctacaaagaaggccggacaggccaagaagaagaagggttcgtacccctacgacgtgcctgactatgcctga

SEQ ID NO: 445 - DNA, 2850 bp, synthetic construct
  misc_feature 1..2850: note = Synthetic: OEC053, TAL34-KRAB
  source 1..2850: mol_type = other DNA
atggccccaaagaagaagcggaaagtcggaggttcaggcggctcgcacatggtggacctgagaactctgggttactcccagcaacagcaagaaaaaatcaagccaaaagtgcgcagcactgtcgcgcagcaccacgaggcccttgtggggcacgggttcacccacgctcacatcgtggccctgagccagcatccggcagccctgggaaccgtcgcagtgaagtaccaagacatgatcgccgccttgccggaagccactcacgaagcaattgtgggagtggggaagagaggagccggcgcaagagccctcgaagcgcttctgactgtggccggagaattgagaggccccccactgcagctggataccggacaactgctgaagatcgccaaacggggtggagtgactgccgtcgaggctgtgcacgcctggagaaacgccttgaccggagcgccactgaaccttaccccggagcaagtggtggcgatagcatcaaatattggcggaaagcaggccctggaaaccgtacaaagactgttgccggtcctgtgtcaagctcatggtttgaccccagaacaggtcgtggctattgcatcgaacggcggaggcaaacaagccctcgagacagtccagagacttctgccggttctgtgccaggcgcacggacttacccctgaacaggtggtggcgattgcgagccacgacggtggtaaacaggcgttggaaactgtgcagcggttgcttccggtgctgtgtcaggctcacgggctgacccccgaacaagtggtagctatcgcctcccacgacggagggaagcaggctctggaaacggtgcaacgcctccttcccgtcttgtgccaagcacatggactgactccagaacaggtcgtcgccattgcctcccatgatggggggaagcaggcactggaaactgtacaacgcctgctgcctgtcctctgccaagcgcacggtttgacgccggaacaggttgtcgccatcgcctcgaacggagggggaaaacaggcccttgaaacagtgcaacggctgctgccagtgctgtgtcaggcgcatggcttgaccccggagcaagtggtggccatagcctcgaatggaggaggaaagcaggccctggaaaccgttcagcgcctcctccctgtgctgtgtcaggcccatgggctcactcccgaacaggtagtcgccatagcgtcgaacatcggaggaaaacaagcgctcgaaactgtccaaagactgcttccagtcctttgccaggctcatggactaactccagagcaagtagtggccatcgcgagccatgacggcggcaagcaagcactcgaaaccgtgcagaggctgcttcccgtcctgtgccaagctcacggcctgacgcctgaacaggtggtggctatcgccagcaacggtggtggaaagcaggcgctcgagactgtgcagaggttgctcccagtgctctgtcaggcacatggcctcaccccggagcaagtggtggcaatcgcctcaaacatcggtggcaaacaggcacttgaaaccgtgcagcgccttttgcctgtgctctgccaggcccacggactgacccctgagcaagtcgtggctattgcctctaacggcggtggcaagcaggccttggaaaccgtccagaggctcttgccggtgctctgccaggcacacggactcactccggaacaggtggtcgccatcgcatcgaatatcggcggaaaacaggccctcgaaacggtccagcggttactccccgtgctttgtcaggctcacggattgactcccgagcaagtcgtggccattgcttccaacattggggggaaacaggcgctggaaacggtgcagcgactccttcctgtgttgtgtcaagcccacggcctcactccagagcaagtcgtggcaattgcctccaacatcgggggaaagcaagccttggagacagtacagcggctgcttcctgtgctttgccaagcccatggactcacccccgaacaagtggttgccattgccagccacgatggagggaaacaagccctggagacagtgcaaaggctcctgcccgtgctgtgccaggcacacggcttgactcctgaacaagtcgtcgctatcgcttccaatggtggtgggaagcaagctctggagactgtccagcggctcctgccagtgttgtgtcaagcacacggtctgaccccggaacaagtcgttgcaatcgcgtcgcatgatggaggacgccctgccctcgagtccattgtggcccagctgtcacgcccagatcctgcactggccgctcttaccaacgatcacctggtcgctctcgcgtgcctgggaggaaggcctgccctggatgccgtcaagaagggactgcctcatgcccctgctttgattaagaggactaatcggcgcatccccgaacggaccagccacagggtggcatctgggagcggaggaggatccggcggagacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcgcccagcggctacaaagaaggccggacaggccaagaagaagaagggttcgtacccctacgacgtgcctgactatgcctga

SEQ ID NO: 446 - DNA, 4092 bp, synthetic construct
  misc_feature 1..4092: note = Synthetic: OEC043, ZF05-KRAB-tPT2A-TAL14-KRAB
  source 1..4092: mol_type = other DNA
atggccccaaagaagaagagaaaggtcggaattcatggcgtgcccgcagccggcagcagcggttccctggagcccggagagaaaccgtataagtgcccggaatgtggaaagtccttctcccaatccggccacctgactgagcatcagcgcacccacactggcgaaaagccgtacaagtgccctgagtgcgggaagtcgtttagccagcgggcgaacttgcgcgctcatcagcggactcataccggggagaagccctataaatgcccggagtgcggaaagagcttcagcaccaagaactccctgactgaacaccagcggacccacaccggagagaagccttacaagtgtcctgaatgcgggaaatccttctccacctccggttcactcgtgcgccaccagagaacgcacaccggtgaaaagccttataagtgtccagagtgcggcaaaagcttcagccagtccggcaacctcaccgagcaccaacggactcacactggagaaaagccctacaaatgtccggaatgcggcaaatcattctcgcaagccggtcaccttgcctcgcaccagcgcacacacaccggggaaaagccatacaaatgccccgaatgcggaaagtcgttctctcaatccggcgacctgagaaggcatcagaggacccataccggaaagaaaacctccgccagcggatcaggaggtggatcaggcggagatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtgtccggtggaaagcggcctgcagcgactaagaaggccggacaggccaagaagaagaagggctcaggctcagcaaccaacttctccttgctcaaacaagccggggacgtcgaagagaaccccggaccgactagcgccggaaagctgggttccggagagggtcgaggttccctccttacctgtggagatgtggaagaaaacccaggacccctggagggctccagcggtagcggaagcccgaagaagaagcggaaggtcggaatccacggagtgcctgccgctggaagctcgggttcccacatggtcgatcttcgcaccctcgggtactcgcagcagcaacaggaaaagatcaagccgaaagtcaggtccaccgtcgcccagcaccacgaggcactggtcggccacggattcacccacgcccacatcgtcgcgctgtctcaacatccagccgccctgggaactgtcgccgtcaagtaccaagatatgatcgcggcactgcctgaagcaacccatgaggccatcgtgggtgtgggaaagagaggagcaggggctagagccctggaagcgctcctgaccgtggcgggagagcttagaggccctcccctgcaattggacaccggccaacttctgaagattgcaaaaaggggtggagtgactgccgtggaagcagtgcacgcttggcggaacgcactgactggagcacccctgaatttgacccccgagcaagtggttgccatcgcaagcaacatcggagggaaacaagcactggaaactgtgcagcgcctgcttccggtcctgtgccaagctcacggactgactcctgaacaggttgtggccatcgcgtcgcatgacgggggaaaacaggcgctcgaaaccgtccaaagactgctcccggtgctgtgtcaggctcacggtttgaccccagaacaagttgtcgctatcgcctcacacgacggcgggaagcaagctctggagactgtgcaaagactccttcctgtgctgtgccaagctcatggattgaccccggaacaggtcgtcgccatcgcttccaatggcggaggtaaacaggccttggaaacggtgcagaggctgctgccagtcttgtgtcaggcgcacgggctgactccagagcaagtagtagccatcgccagcaatggaggcggtaaacaagcgctcgagaccgtccagcgcttgttgccagtgttgtgccaggcccatgggctgacgcccgaacaggtggtggccattgcatccaacattggggggaaacaggcgttggagacagtccaacggcttctgcccgtcctttgtcaggcacacggactcactcctgaacaagtcgttgccattgcctcgaatatcgggggtaagcaagccctcgagactgtgcagagattgttgcctgtcctgtgtcaagcgcatggtctgactcccgaacaggtggtggcgattgcttcgaacaatggaggaaaacaagcattggaaaccgtccagaggctgttaccagtcctttgccaggcacatggactgaccccggaacaggtcgtcgcaatcgcatcgaacattggaggcaagcaggctctcgaaacggtccagagactgctgcctgtgctctgccaagcccacggcctcactccggagcaagtcgtggcaatcgcctcaaacatcggtggaaagcaggcccttgagactgtccagcgactgctcccagtgctctgccaggcgcatggactcacacccgagcaggtggtcgccattgctagcaacaacggaggcaaacaagccctggagacagtgcaacgcttgctgcccgtgctttgtcaagcgcacggacttactccggaacaagtagtggctattgcgagtaggcacggcggtaagcaagccctggagacagtgcagcggcttttgccggtgctctgtcaggcccacgggctcacaccggaacaggtcgtagctattgcgtcccatgacggcggaaagcaagccttagaaactgtgcagcggttgctgcctgttctgtgccaggctcatgggctcactcccgagcaagtggtcgcgattgcgagcaacggaggtggcaagcaagcactagaaaccgtgcaacgactgctgccggtcttgtgccaagcgcatggcctgacacccgaacaggtggtggctatcgcgtccaacaacgggggcaaacaggccctggaaacagtccaaaggctgttgccggtcctctgtcaagctcacggactaacccccgaacaggtggtggcaatagcttcgcggcatgggggcaagcaggcactcgaaacagtgcagaggttgctcccggtactttgccaagcacatggcttgacccctgagcaagtcgtggccatcgcatccaacggcggagggaagcaagcgcttgaaactgtccaacgcctcctgcccgtgttgtgtcaagcccatggccttacgcctgaacaagtcgtcgctattgcaagtaacggaggagggaggcctgccctggagtccattgtcgcccaactgtcgcgacccgatccagccctggcagccctcactaacgaccatctcgtggcgctggcatgcctcggtggtagaccagctctggacgccgtgaagaagggacttcctcatgcccccgcactgatcaagcgcactaaccggaggatccctgaaagaacctcccacagagtggcatccggttcaggaggagggtccggaggagctagggatgatgccaagagcttgaccgcctggtcacggactcttgtcactttcaaggatgtctttgtggactttaccagagaagaatggaagctcctggacactgcgcagcagatcctgtacagaaacgtgatgttggaaaactacaaaaacctggtgtcactcgggtaccaactcaccaagcccgacgtcatcctgcgcctggaaaagggcgaagaaccgtggttggtcgagagagaaatccatcaggaaacccaccctgactcggaaaccgccttcgagatcaagtcctcggtgcctagctccggaggaaaaaggcccgccgcaaccaagaaggctgggcaggctaagaaaaagaaggggtccggctcctaatga

SEQ ID NO: 447 - RNA, 1361 nt, synthetic construct
  misc_feature 1..1361: note = Synthetic: MR250, ZF05-KRAB
  source 1..1361: mol_type = other RNA
aggaaataagagagaaaagaagagtaagaagaaatataagagccaccatggcccccaagaagaagcggaaggtgggcatccacggcgtgcccgccgccggcagcagcggatccctggagcccggagagaaaccgtataagtgcccggaatgtggaaagtccttctcccaatccggccacctgactgagcatcagcgcacccacactggcgaaaagccgtacaagtgccctgagtgcgggaagtcgtttagccagcgggcgaacttgcgcgctcatcagcggactcataccggggagaagccctataaatgcccggagtgcggaaagagcttcagcaccaagaactccctgactgaacaccagcggacccacaccggagagaagccttacaagtgtcctgaatgcgggaaatccttctccacctccggttcactcgtgcgccaccagagaacgcacaccggtgaaaagccttataagtgtccagagtgcggcaaaagcttcagccagtccggcaacctcaccgagcaccaacggactcacactggagaaaagccctacaaatgtccggaatgcggcaaatcattctcgcaagccggtcaccttgcctcgcaccagcgcacacacaccggggaaaagccatacaaatgccccgaatgcggaaagtcgttctctcaatccggcgacctgagaaggcatcagaggacccataccggaaagaaaacctccgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 448 - RNA, 1361 nt, synthetic construct
  misc_feature 1..1361: note = Synthetic: MR319, ZF07-KRAB
  source 1..1361: mol_type = other RNA
aggaaataagagagaaaagaagagtaagaagaaatataagagccaccatggcccccaagaagaagcggaaggtgggcatccacggcgtgcccgccgccggcagcagcggatccctcgagcctggcgaaaagccttataagtgcccagaatgcggaaagagcttctccgacaagaaggatctgacccgccaccaaaggacccacactggggagaagccttacaagtgtccggagtgcggaaagtccttttcgcaatcgtccagcctggtccggcaccagaggacgcacaccggagagaagccatacaaatgcccggagtgtgggaagtcgttctccaccactggaaacctcactgtgcaccagcgcacgcatactggggaaaagccctataagtgtccagagtgcgggaagtccttctcaacctccggcgaattggtcagacaccagcggacacacaccggcgagaagccctacaagtgccctgagtgcggcaaaagcttcagcactaccggcaacctgactgtgcatcagcggacccataccggagaaaaaccctacaaatgtcccgaatgcggcaaatccttctccacctcccactcgctgaccgaacaccagagaacccatactggagaaaagccgtacaagtgcccggaatgtggaaagtctttcagcacctccggaaaccttgtgcggcatcagcgcactcacaccggcaaaaagacctcagcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 449 - RNA, 2243 nt, synthetic construct
  misc_feature 1..2243: note = Synthetic: MR340, ZF05-MQ1
  source 1..2243: mol_type = other RNA
aggaaataagagagaaaagaagagtaagaagaaatataagagccaccatggcccccaagaagaagcggaaggtgggcatccacggcgtgcccgccgccggcagcagcggatccctggagcccggagagaaaccgtataagtgcccggaatgtggaaagtccttctcccaatccggccacctgactgagcatcagcgcacccacactggcgaaaagccgtacaagtgccctgagtgcgggaagtcgtttagccagcgggcgaacttgcgcgctcatcagcggactcataccggggagaagccctataaatgcccggagtgcggaaagagcttcagcaccaagaactccctgactgaacaccagcggacccacaccggagagaagccttacaagtgtcctgaatgcgggaaatccttctccacctccggttcactcgtgcgccaccagagaacgcacaccggtgaaaagccttataagtgtccagagtgcggcaaaagcttcagccagtccggcaacctcaccgagcaccaacggactcacactggagaaaagccctacaaatgtccggaatgcggcaaatcattctcgcaagccggtcaccttgcctcgcaccagcgcacacacaccggggaaaagccatacaaatgccccgaatgcggaaagtcgttctctcaatccggcgacctgagaaggcatcagaggacccataccggaaagaaaacctccgcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 450 - RNA, 2243 nt, synthetic construct
  misc_feature 1..2243: note = Synthetic: MR353, ZF07-MQ1
  source 1..2243: mol_type = other RNA
aggaaataagagagaaaagaagagtaagaagaaatataagagccaccatggcccccaagaagaagcggaaggtgggcatccacggcgtgcccgccgccggcagcagcggatccctcgagcctggcgaaaagccttataagtgcccagaatgcggaaagagcttctccgacaagaaggatctgacccgccaccaaaggacccacactggggagaagccttacaagtgtccggagtgcggaaagtccttttcgcaatcgtccagcctggtccggcaccagaggacgcacaccggagagaagccatacaaatgcccggagtgtgggaagtcgttctccaccactggaaacctcactgtgcaccagcgcacgcatactggggaaaagccctataagtgtccagagtgcgggaagtccttctcaacctccggcgaattggtcagacaccagcggacacacaccggcgagaagccctacaagtgccctgagtgcggcaaaagcttcagcactaccggcaacctgactgtgcatcagcggacccataccggagaaaaaccctacaaatgtcccgaatgcggcaaatccttctccacctcccactcgctgaccgaacaccagagaacccatactggagaaaagccgtacaagtgcccggaatgtggaaagtctttcagcacctccggaaaccttgtgcggcatcagcgcactcacaccggcaaaaagacctcagcatctgggagcggaggaggatccggcggagccagagactccaaggtcgagaacaagaccaagaagctgcgggtgttcgaggcctttgccgggatcggagcacagcggaaggccttggagaaggtccgcaaggacgaatacgaaatcgtgggccttgccgagtggtacgtgccagccatcgtgatgtaccaggctatccacaacaacttccacaccaagctggagtacaagtccgtgagcagagaagagatgatcgactacctcgaaaacaagaccctgtcctggaactccaagaatcccgtgtcgaacggttactggaagcgcaagaaggatgatgaactgaagattatctacaacgcgattaagctgtccgaaaaggaaggaaacattttcgacattcgcgatctgtacaagagaaccctcaagaacattgatctgcttacctactccttcccgtgccaagatctcagccagcaggggattcagaaggggatgaagcggggttcagggaccagatcaggcctgctctgggagatcgaaagagccctggactccaccgagaagaacgatctcccgaaataccttctgatggagaacgtcggcgctctcctgcacaagaagaacgaagaagaactgaaccagtggaagcagaagctggagtcactcggataccagaattctattgaggtgctgaacgcggccgatttcggatcctcgcaagccaggcggagagtgttcatgatttcgaccctgaacgagttcgtggagctgcctaagggcgacaagaagcccaagtccatcaagaaggtcctgaacaagattgtcagcgaaaaggacatcctcaacaatctgcttaagtacaacctgaccgagtttaaaaagaccaaaagcaacatcaacaaggcctccctgatcggatactccaagttcaattccgaaggctacgtgtacgacccggaattcaccggaccaaccttgaccgcctccggtgctaactcccggatcaagatcaaggacggatccaacatccggaagatgaactccgacgagacttttctgtacatcggtttcgactcccaagacggaaagcgcgtgaacgagatagagttcctcaccgagaatcagaagatctttgtgtgcggcaactccattagcgtggaggtgctggaggccatcatcgataagatcggtggcccttcctccggtggaaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 451 - RNA, 1361 nt, synthetic construct
  misc_feature 1..1361: note = Synthetic: MR553, ZF16-KRAB
  source 1..1361: mol_type = other RNA
aggaaataagagagaaaagaagagtaagaagaaatataagagccaccatggcccccaagaagaagcggaaggtgggcatccacggcgtgcccgccgccggcagcagcggatccctggagccgggagagaagccttataagtgtcctgagtgcggcaaatcattctcccaacgcgcccacctcgagagacaccagagaacccacaccggggaaaagccgtacaagtgccctgagtgtgggaagtccttttcccggtccgaccatttgaccaaccatcagcggacccacactggggagaagccatacaaatgcccagaatgcggcaaaagcttctcccggaacgacgcacttaccgaacatcagcgcacgcacaccggagagaaaccctataagtgccccgaatgtggaaagtcattctcccgggccgacaacctgacagagcaccagcggactcacactggagaaaagccttacaagtgtcccgaatgcgggaagtcgttcagctcgaagaaggctctgaccgaacaccagaggacccataccggcgaaaagccctataaatgcccggagtgcggaaagagcttcagcaggaacgatgccctcaccgagcaccaacgcactcatactggcgaaaaaccgtacaaatgtccggaatgcggaaagtccttctcgcacaagaacgcgctgcagaatcaccagcgcactcacaccggcaaaaagacctctgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 452 - RNA, 1361 nt, synthetic construct
  misc_feature 1..1361: note = Synthetic: MR697, ZF24-KRAB
  source 1..1361: mol_type = other RNA
aggaaataagagagaaaagaagagtaagaagaaatataagagccaccatggcccccaagaagaagcggaaggtgggcatccacggcgtgcccgccgccggcagcagcggatccctggaacccggagagaagccctataagtgtccggaatgcggaaagagtttcagctcgaagaagcacctcgccgagcaccaacggacccacacgggagaaaagccctacaagtgcccagagtgcggaaagtcgttcagccaaaactccaccctgaccgaacaccagcggactcataccggggagaaaccttacaagtgtccagaatgcgggaagtcgttctcccaatccggagacttgcgcagacaccagcgcactcacaccggcgaaaaaccgtacaaatgcccggagtgtggaaagtcattttccacccacctggatctgatcaggcatcagcgcacccacactggcgaaaagccttacaaatgtccggagtgcggcaaatccttctctgaccccggcaacctcgtgcgccatcagaggactcacactggggagaagccttataagtgccccgaatgtggaaagagcttctccacctccggcaaccttaccgagcatcagcggacacacaccggggaaaagccgtacaagtgccctgagtgtggcaaatcattctccgaacggagccatctgcgggagcaccagagaacccacaccggaaagaaaacttccgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 453 - RNA, 3143 nt, synthetic construct
  misc_feature 1..3143: note = Synthetic: MR896, TAL04-KRAB
  source 1..3143: mol_type = other RNA
aggaaataagagagaaaagaagagtaagaagaaatataagagccaccatggcccccaagaagaagcggaaggtgggcatccacggcgtgcccgccgccggcagcagcggatcccacatggtggacctgagaaccctgggatactcgcagcagcagcaggaaaagatcaagcccaaagtgcggtccactgtggcccagcaccatgaggccctcgtcggacacggattcacccacgcgcacatcgtggctctgtcacagcatcccgccgccttgggaaccgtggctgtgaagtaccaggacatgattgctgcacttcctgaggcgacccacgaagccattgtgggagtcggaaaacggggtgcaggcgctagagccctggaggccctgttaacggtggcaggcgaactgcggggaccaccattgcagctcgataccggccagctgctgaagattgccaagaggggcggagtcacagcggtggaggcggtccatgcctggcgcaacgctcttactggggctccactgaacctgacccctgaacaggtagtggccatcgcatcgaacaatggcggtaagcaggctctcgaaaccgtccaaaggctccttccggtgttatgccaggctcatgggctgacgcctgaacaagtggtcgcaatcgcatcgcatgacggggggaaacaggcattagaaaccgtgcaacgcctccttcctgtcctttgtcaagcacatggattgactcctgagcaagttgtcgcgattgcctccaacattggcggcaaacaggcactggaaaccgttcaacggctcctccccgtactgtgccaggcacacggcctgacccccgaacaagtcgtcgccattgcctcgaacatcggaggcaagcaggcgcttgaaaccgtgcagcgcttgctgcctgtgctctgccaagcgcacggcctaacccctgaacaggtggtagcaattgcttcccacgacggtgggaagcaggccctcgagacagtgcaaagactgttgccggtcctctgccaggcccacggacttacccccgagcaagtcgtggctattgcgtccaataatggcggaaaacaggcacttgagacagtccagaggctgctgcctgtcttatgccaagcccacggtcttactccagaacaggtggtcgctatcgcatcaaacattggaggaaaacaggcgctggaaactgtccaacgcctgttgcccgtgctctgtcaggctcacgggctgactcccgagcaagtggtggcaatcgcctccaacgggggaggaaagcaggccttggaaaccgtacagcggctgttgcctgtgttgtgccaagctcacggtctgacgccagagcaagtggtggccattgcttcgcacgacggcgggaagcaggcactcgaaacggtgcagaggttgcttcccgtcttgtgccaggcgcacggactcaccccagaacaagtggtcgcaattgctagcaacattggtggcaaacaagcgctcgaaactgtacaacggctgctcccggtgctttgccaagcccacgggcttaccccagagcaagtggtggccattgcgagcaacatcgggggcaagcaagcgcttgagactgtacaaagactgctgcccgtgctgtgccaagcccacggcttaaccccggaacaggtcgtggccatcgcctcgaatggaggtggaaagcaagccctggagactgtgcagcggttgctgcccgttctgtgtcaagctcacggactgacaccggaacaggtggtggctatcgcaagcaacaacggcggaaagcaagccctggagactgtgcagcggctgctgccagtgctgtgtcaggcgcatggactgacccccgaacaagtcgtagccatagccagccgccacggagggaagcaggccctggaaacagtccagcgcctcttgcccgtgttgtgtcaagcgcatggcctcacccctgaacaggtcgttgccattgcgtccagacatggagggaagcaagcactagaaacagtgcagagactgctacccgtcctgtgccaggcccatggactcacgccggagcaagtggtggcgatagcatcgaacattggtgggaaacaagcccttgaaactgtccagagactgttacctgtgctttgtcaggcacatgggctcacaccagaacaggtggtcgccatcgctagcaacggagggggaaaacaggctttggagactgtccagcggctcctaccggtgctctgccaagcacacggattgacgcccgaacaggttgtggcgattgcgtccaacggtggaggacggcctgccctggagagcattgtggctcagctctccagacctgacccagccctcgcagctctgactaacgatcatctggtggcgctggcctgtttggggggacgccctgctttggacgccgtgaagaagggtctgccacatgcccccgcactgatcaagcgcaccaaccggcgaatccctgagcgcacttcccatcgagtggcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 454 - RNA, 3143 nt, synthetic construct
  misc_feature 1..3143: note = Synthetic: MR861, TAL08-KRAB
  source 1..3143: mol_type = other RNA
aggaaataagagagaaaagaagagtaagaagaaatataagagccaccatggcccccaagaagaagcggaaggtgggcatccacggcgtgcccgccgccggcagcagcggatcccacatggtggacctccggactcttggttactcccaacagcagcaggaaaagatcaagcctaaggtccgctccactgtggcccaacaccacgaggcccttgtgggacatggattcacccacgcccacatcgtggcgctgtcgcagcaccctgctgccttggggaccgtcgccgtgaagtaccaggacatgatcgccgccttacccgaagccacccacgaggcaatcgtgggagtgggtaaaaggggtgccggtgcaagagctctggaggcccttctcaccgtggccggagaacttagagggcctccactccagctggataccggtcagctgttgaagattgctaagagaggaggggtgaccgccgtggaagccgtgcatgcgtggcgcaacgctctgactggagccccactgaacttgacccccgaacaggtggtcgccatcgcctccaataacgggggcaaacaggcgctggaaaccgtgcagagattgctgcctgtcttatgccaagcacacggactcacccccgagcaagtcgtggctatcgcctccagacacggcgggaagcaagctctggagactgtgcagagactgctacccgtgttgtgtcaagcccacggtcttactccggagcaagtggtcgctattgcctcgcatgatggtggtaaacaggccctggagacagtgcagcgcttgctccctgtgctgtgtcaggcacacggcctgacaccggaacaggtggtggccatcgcttcgaacattggaggtaaacaggcattggagacagtccagaggctcctgccggtcctctgccaagctcacggtctgactcctgaacaagtagtcgccattgcatcccacgatggcggtaagcaggctcttgaaactgtccagagactgttacctgtgctctgtcaggcccatggactgacgcctgaacaggtcgtcgcgattgcgtcaaacatcggtggaaagcaagcgcttgaaacggtccagcgcctactgcctgtgttgtgccaagcgcacggcctcacaccagaacaagtcgtcgcaatcgcaagccatgatggcggaaagcaggcactagaaactgttcagcgccttctacccgtcttgtgccaggctcatggactcactcccgaacaagtggtggctattgcgtccaatggaggcggtaaacaagccctcgaaaccgtccagcggcttctgccggttctgtgccaggcacatggcttgactcctgagcaagttgtcgcgattgcctccaacattgggggcaagcaagcactcgaaacagtgcagaggcttctaccagtgctctgccaggcccacggtttgacccctgaacaggtggtcgccatcgcctccaacaacggcggaaaacaagcactggaaactgtgcaaagactgcttcctgtgctgtgtcaggcgcatggcctcaccccggaacaagtggtggccatagcgtcgcacgacggaggcaagcaggcccttgaaacggtgcagcgactgttgcccgtcctttgtcaggctcacgggttaaccccagaacaggtcgtggcaatcgcctctcatgacggggggaagcaggccttggagactgtccaaaggttgctgccagtgctgtgccaagcccacgggctcactccagagcaagtagtggcgatagcgagccacgacggcgggaaacaggctctggaaaccgtacagaggttgctccccgtgctttgtcaagcacatggtctgacccccgagcaagtcgtggccattgccagccatgacggagggaaacaggcacttgaaaccgtccaaaggctgctgcctgtgctttgccaggcgcatgggcttactcctgaacaagtggtggcgattgcttcaaatatcggtggcaaacaagctttggaaacagtccagcggttgctcccagtcctctgtcaagctcacgggctgacgccagaacaggtcgtggcaattgcgtcacacgacggaggaaaacaggcgttggaaaccgttcagcggctcttgcccgtgctatgccaggcccacggactgactccggaacaggtggttgccatcgccagcaacaacggagggaagcaggcgctcgagactgtacagcgacttctccccgtcctgtgtcaagcccatggcctgacgccggaacaggtggtagccattgcgagcaacggaggaggacgcccagccctagagagcatcgtggcccaactgtcgcggccggatccagctctggccgcattgaccaacgaccacctggtcgctctggcttgtctgggaggacgaccggccctggatgccgtgaagaagggcctgccacatgcccctgccctcattaagcggactaacagaaggatccccgagaggacctcacacagagtcgcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 455 - RNA, 3143 nt, synthetic construct
  misc_feature 1..3143: note = Synthetic: MR853, TAL09-KRAB
  source 1..3143: mol_type = other RNA
aggaaataagagagaaaagaagagtaagaagaaatataagagccaccatggcccccaagaagaagcggaaggtgggcatccacggcgtgcccgccgccggcagcagcggatcccacatggtggacctccggaccctgggatactcccagcagcagcaggaaaagatcaagccgaaggtccgctccactgtggctcaacaccacgaagcccttgtgggacacggcttcactcatgcacacattgtggcattgtcccaacaccctgccgctctgggaactgtggcagtgaagtaccaggacatgatagccgcactgcccgaagcaacacacgaggccatcgtcggtgtgggaaaaagaggagcgggagccagagcactcgaagcgctcctgacagtggcgggagaactccgcggaccacctctgcagctggacactggccagctgctgaagattgcaaaacgcggtggagtcaccgctgtggaggcggtgcatgcctggagaaatgccctgactggtgcccccttgaacctgacccccgagcaagtcgtcgcaattgcttcgaacaacggtggtaaacaggctctcgaaaccgtccaacggctgctcccagtcctatgccaagcccatggacttacgcccgaacaagtggttgctatagcctccaatatcggtgggaagcaggctctggagacagtccaaagactcctgccggtcctctgccaagcgcacggactcaccccagagcaagtagtggccattgcctcccacgatggtggcaagcaggcccttgaaacagtccagagactgttacccgtgctttgtcaagcccacgggctcactcccgaacaggtggtggcgattgcctctaacggcggcggcaaacaggcgctcgagactgtccagcggcttctaccggtgttgtgccaggcccatggtctgacacccgaacaggtggttgcaattgcctccaacggaggcgggaagcaagccctcgaaacggtccagcggttgttgccggtcctgtgtcaggcgcacgggttgaccccagaacaggtcgtcgctattgcctcaaacatcggtggtaagcaggcgctggaaactgtgcagcgactcctgcctgtgctctgtcaagcgcacgggcttacgccagaacaagtggtcgctatcgcgtccaacaacggcggcaagcaagctctggaaaccgtccaaaggctcttgcccgtcctatgtcaggcccacggcctcacccctgaacaggttgtcgccatcgccagccacgacggcggtaagcaagccttggaaaccgtgcaacgactgcttcccgtgttgtgtcaggctcatggcctcactcctgagcaagttgtggccatcgcttcgaatattgggggcaaacaagccctggaaacggtgcagcggttgctgcctgtcttgtgtcaagctcacgggctgactcctgaacaagtcgtggccatagcgtccaacattgggggaaagcaggcgttggaaactgtccagaggctgttgcccgtgctctgccaggctcacggcctgacccctgaacaggtggtcgcaatcgcctccaacatcggagggaagcaggcactggaaacagtgcagcggctgctgcccgtgctgtgccaagctcatggactcactccggaacaggtcgttgcaatcgcatcaaacattggaggaaagcaggccctcgagacagtgcagagactgctccctgtgctttgccaggcgcatggccttaccccggaacaagtggtagccatcgcgtcgcatgacggcggaaagcaggctttggaaaccgtgcaaaggctcctgcccgtcctgtgccaggctcacggtctgaccccggaacaagtcgtggccatcgctagccatgatggagggaaacaggccctggagactgtacagagactcctccctgtcctttgccaagcacacggacttacccctgaacaggtcgtggctattgcatcgaacgggggggggaaacaagcgctcgaaactgtgcaacgcctcctgccagtgttgtgccaggcacatggcttgactcccgagcaagtcgtcgccattgcatcaaacaacggaggaaaacaggcactcgagactgtgcaaagactgttgccggtgctatgccaggcacacggcttgaccccggagcaagtggtggctatcgcctcgcacgatggcggaaaacaagcactcgaaactgttcagcggctactgccggtgctctgccaggctcacggactgactccagaacaggtggtcgcaatcgcgtcgaacggtggaggacggcctgccctcgagagcattgtggcgcagctctcccgcccggatcctgctttggccgcgctgaccaacgaccacctggtggccctcgcctgtttggggggtcggcctgctttggacgccgtcaagaagggcctgcctcatgcccccgcactcatcaagcggaccaatagacgcatcccggaaaggacttctcaccgggtggcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 456 - RNA, 3143 nt, synthetic construct
  misc_feature 1..3143: note = Synthetic: MR836, TAL14-KRAB
  source 1..3143: mol_type = other RNA
aggaaataagagagaaaagaagagtaagaagaaatataagagccaccatggcccccaagaagaagcggaaggtgggcatccacggcgtgcccgccgccggcagcagcggatcccacatggtcgatcttcgcaccctcgggtactcgcagcagcaacaggaaaagatcaagccgaaagtcaggtccaccgtcgcccagcaccacgaggcactggtcggccacggattcacccacgcccacatcgtcgcgctgtctcaacatccagccgccctgggaactgtcgccgtcaagtaccaagatatgatcgcggcactgcctgaagcaacccatgaggccatcgtgggtgtgggaaagagaggagcaggggctagagccctggaagcgctcctgaccgtggcgggagagcttagaggccctcccctgcaattggacaccggccaacttctgaagattgcaaaaaggggtggagtgactgccgtggaagcagtgcacgcttggcggaacgcactgactggagcacccctgaatttgacccccgagcaagtggttgccatcgcaagcaacatcggagggaaacaagcactggaaactgtgcagcgcctgcttccggtcctgtgccaagctcacggactgactcctgaacaggttgtggccatcgcgtcgcatgacgggggaaaacaggcgctcgaaaccgtccaaagactgctcccggtgctgtgtcaggctcacggtttgaccccagaacaagttgtcgctatcgcctcacacgacggcgggaagcaagctctggagactgtgcaaagactccttcctgtgctgtgccaagctcatggattgaccccggaacaggtcgtcgccatcgcttccaatggcggaggtaaacaggccttggaaacggtgcagaggctgctgccagtcttgtgtcaggcgcacgggctgactccagagcaagtagtagccatcgccagcaatggaggcggtaaacaagcgctcgagaccgtccagcgcttgttgccagtgttgtgccaggcccatgggctgacgcccgaacaggtggtggccattgcatccaacattggggggaaacaggcgttggagacagtccaacggcttctgcccgtcctttgtcaggcacacggactcactcctgaacaagtcgttgccattgcctcgaatatcgggggtaagcaagccctcgagactgtgcagagattgttgcctgtcctgtgtcaagcgcatggtctgactcccgaacaggtggtggcgattgcttcgaacaatggaggaaaacaagcattggaaaccgtccagaggctgttaccagtcctttgccaggcacatggactgaccccggaacaggtcgtcgcaatcgcatcgaacattggaggcaagcaggctctcgaaacggtccagagactgctgcctgtgctctgccaagcccacggcctcactccggagcaagtcgtggcaatcgcctcaaacatcggtggaaagcaggcccttgagactgtccagcgactgctcccagtgctctgccaggcgcatggactcacacccgagcaggtggtcgccattgctagcaacaacggaggcaaacaagccctggagacagtgcaacgcttgctgcccgtgctttgtcaagcgcacggacttactccggaacaagtagtggctattgcgagtaggcacggcggtaagcaagccctggagacagtgcagcggcttttgccggtgctctgtcaggcccacgggctcacaccggaacaggtcgtagctattgcgtcccatgacggcggaaagcaagccttagaaactgtgcagcggttgctgcctgttctgtgccaggctcatgggctcactcccgagcaagtggtcgcgattgcgagcaacggaggtggcaagcaagcactagaaaccgtgcaacgactgctgccggtcttgtgccaagcgcatggcctgacacccgaacaggtggtggctatcgcgtccaacaacgggggcaaacaggccctggaaacagtccaaaggctgttgccggtcctctgtcaagctcacggactaacccccgaacaggtggtggcaatagcttcgcggcatgggggcaagcaggcactcgaaacagtgcagaggttgctcccggtactttgccaagcacatggcttgacccctgagcaagtcgtggccatcgcatccaacggcggagggaagcaagcgcttgaaactgtccaacgcctcctgcccgtgttgtgtcaagcccatggccttacgcctgaacaagtcgtcgctattgcaagtaacggaggagggaggcctgccctggagtccattgtcgcccaactgtcgcgacccgatccagccctggcagccctcactaacgaccatctcgtggcgctggcatgcctcggtggtagaccagctctggacgccgtgaagaagggacttcctcatgcccccgcactgatcaagcgcactaaccggaggatccctgaaagaacctcccacagagtggcttccggatccggtggaggatcaggcggcgatgcgaagtccctcaccgcttggtcacggaccctagtgaccttcaaagacgtgttcgtggatttcaccagggaagaatggaagctcctggacaccgcccagcaaatactgtaccggaacgtcatgctggagaactataagaacctggtcagcttgggctaccagctcaccaagccggacgtgatcctgcggctggagaagggagaggagccttggctcgtggaaagggaaattcatcaggagactcaccctgacagcgaaaccgccttcgagattaagtcctccgtgtccggaggaaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 457 - RNA, 3143 nt, synthetic construct
  misc_feature 1..3143: note = Synthetic: MR942, TAL27-KRAB mouse
  source 1..3143: mol_type = other RNA
aggaaataagagagaaaagaagagtaagaagaaatataagagccaccatggcccccaagaagaagcggaaggtgggcatccacggcgtgcccgccgccggcagcagcggatcccacatggtggacctccgcacgctgggatactcgcaacagcagcaggagaagattaagcctaaggtccggtccaccgtggctcagcaccatgaggcgctagtgggccacggattcacccacgcgcatattgtcgccctctcgcaacatcccgccgcactgggaaccgtcgccgtgaagtaccaggacatgattgccgccctgcctgaagctacccatgaagccattgtgggcgtgggaaagagaggagcaggggccagagccttggaggccctgctcacggtagccggagagctcagaggcccaccactgcagcttgacaccggccaacttctgaagatcgccaagcggggaggagtgaccgccgtggaggcagtgcatgcctggcgcaacgccttgactggagccccgctgaacctgactccggagcaagtggtggctatcgcgtccaacatcggaggaaaacaggccctggaaacggtgcaaagactgctgccggtcctctgtcaagcgcacggactcactcccgagcaagttgtggctatcgcttccaacatcggggggaaacaggctctggaaactgtccaaaggctgttacctgtgctgtgccaagcacacggactgaccccagagcaagtggtcgccatagcctccaacaacggggggaagcaagccctggagactgtgcagcggttgctccccgtcctgtgccaggcccacggattgactcccgaacaagtggtggccattgcgtcaaacatcgggggcaagcaagcgttggaaacagtccaacggttgcttcccgtcctttgccaggcccacggtctgaccccggagcaagtcgtcgccattgctagtaacgggggaggaaagcaagctcttgaaaccgtccagcggcttcttccggtgctctgccaagcacacggcctcacacccgagcaagtagtggccattgcctcaaacattgggggaaagcaggcattggaaactgtgcaacggctgcttcctgtcttgtgccaggcgcatgggctgactccagaacaggtggtcgctatcgcgagtaacaacggtggcaaacaggcactggaaaccgtgcagaggctcttgcctgtcctgtgtcaggcacacggactgacgccagaacaggtggtcgctattgcgagcaatatcggcggaaagcaggcgttagaaactgtgcagagactgctcccagtgctatgccaggctcatgggttgactcctgaacaagtggtggctatcgcctcaaacggtggcggtaaacaggcgcttgagacagtgcaaaggctccttcccgtgttgtgccaagcgcatggcctgacccctgaacaggtcgtggctatcgcgtcaaacaacggaggcaagcaggccctagaaacggtacaacggctgctgcccgtactgtgccaggcccacggactgaccccagaacaggtggtcgcaattgcctccaacattggcgggaagcaggcgctagaaaccgtgcagcggcttctgcccgtcctctgccaggcccacggtctgactcctgagcaagtcgtcgcaatcgccagcaacatcggcggaaaacaagccctcgaaaccgtccagcgactgctgcctgtcctatgccaggcccacgggttgaccccggaacaggtcgtcgccattgcttccaataatggaggcaaacaggcactcgaaacggtgcagagacttctgccggtgctgtgtcaagctcacggcttgacccccgagcaagtggtggcaatcgcttcacgacacggtggaaaacaggccctggagacagtccagagactgctccccgtgctttgccaagctcacggacttactccggaacaagtcgtggcgattgcgtccaacggtggtggaaagcaggccttggaaacggtccagagactgttgccggtcttgtgccaggcgcatggtctgacccccgaacaagtcgtggcaattgcatcccatgacggcgggaaacaggctttggagactgtccagcgcctgttgccagtcctttgtcaagcccacgggttgacacccgaacaggtcgtcgcaatcgcctcgaacatcggtggcaagcaggcccttgaaactgtccaacgcctcctcccggtgttgtgtcaggctcacggcctgactccagaacaagtcgtggccatagcgtcacacgacggtggacggccagccctggaatccatcgtggcgcagctgtccagacccgacccagcactggcggccctcactaacgatcaccttgtggccctggcctgtctgggaggtagaccagccctcgacgctgtgaagaagggactgccgcatgcaccagccctgatcaagcgaaccaaccggcgcattcccgagagaacttcacacagagtggctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 458 - RNA, 4363 nt, synthetic construct
  misc_feature 1..4363: note = Synthetic: MR968, ZF05-KRAB-tPT2A-TAL14-KRAB
  source 1..4363: mol_type = other RNA
ggaaataagagagaaaagaagagtaagaagaaatataagagccaccatggccccaaagaagaagagaaaggtcggaattcatggcgtgcccgcagccggcagcagcggttccctggagcccggagagaaaccgtataagtgcccggaatgtggaaagtccttctcccaatccggccacctgactgagcatcagcgcacccacactggcgaaaagccgtacaagtgccctgagtgcgggaagtcgtttagccagcgggcgaacttgcgcgctcatcagcggactcataccggggagaagccctataaatgcccggagtgcggaaagagcttcagcaccaagaactccctgactgaacaccagcggacccacaccggagagaagccttacaagtgtcctgaatgcgggaaatccttctccacctccggttcactcgtgcgccaccagagaacgcacaccggtgaaaagccttataagtgtccagagtgcggcaaaagcttcagccagtccggcaacctcaccgagcaccaacggactcacactggagaaaagccctacaaatgtccggaatgcggcaaatcattctcgcaagccggtcaccttgcctcgcaccagcgcacacacaccggggaaaagccatacaaatgccccgaatgcggaaagtcgttctctcaatccggcgacctgagaaggcatcagaggacccataccggaaagaaaacctccgccagcggatcaggaggtggatcaggcggagatgccaagtcactgaccgcatggtcccggacccttgtgaccttcaaggacgtgttcgtggacttcaccagagaggagtggaagctgctggacaccgcgcaacagattctctaccggaacgtgatgctggagaactacaagaatctggtgtccctgggctaccagctgaccaagccggacgtgatcctccgcctcgaaaagggggaggagccttggctcgtggagcgggaaatccaccaggagactcacccggactccgagactgccttcgaaatcaagtcatcggtgtccggtggaaagcggcctgcagcgactaagaaggccggacaggccaagaagaagaagggctcaggctcagcaaccaacttctccttgctcaaacaagccggggacgtcgaagagaaccccggaccgactagcgccggaaagctgggttccggagagggtcgaggttccctccttacctgtggagatgtggaagaaaacccaggacccctggagggctccagcggtagcggaagcccgaagaagaagcggaaggtcggaatccacggagtgcctgccgctggaagctcgggttcccacatggtcgatcttcgcaccctcgggtactcgcagcagcaacaggaaaagatcaagccgaaagtcaggtccaccgtcgcccagcaccacgaggcactggtcggccacggattcacccacgcccacatcgtcgcgctgtctcaacatccagccgccctgggaactgtcgccgtcaagtaccaagatatgatcgcggcactgcctgaagcaacccatgaggccatcgtgggtgtgggaaagagaggagcaggggctagagccctggaagcgctcctgaccgtggcgggagagcttagaggccctcccctgcaattggacaccggccaacttctgaagattgcaaaaaggggtggagtgactgccgtggaagcagtgcacgcttggcggaacgcactgactggagcacccctgaatttgacccccgagcaagtggttgccatcgcaagcaacatcggagggaaacaagcactggaaactgtgcagcgcctgcttccggtcctgtgccaagctcacggactgactcctgaacaggttgtggccatcgcgtcgcatgacgggggaaaacaggcgctcgaaaccgtccaaagactgctcccggtgctgtgtcaggctcacggtttgaccccagaacaagttgtcgctatcgcctcacacgacggcgggaagcaagctctggagactgtgcaaagactccttcctgtgctgtgccaagctcatggattgaccccggaacaggtcgtcgccatcgcttccaatggcggaggtaaacaggccttggaaacggtgcagaggctgctgccagtcttgtgtcaggcgcacgggctgactccagagcaagtagtagccatcgccagcaatggaggcggtaaacaagcgctcgagaccgtccagcgcttgttgccagtgttgtgccaggcccatgggctgacgcccgaacaggtggtggccattgcatccaacattggggggaaacaggcgttggagacagtccaacggcttctgcccgtcctttgtcaggcacacggactcactcctgaacaagtcgttgccattgcctcgaatatcgggggtaagcaagccctcgagactgtgcagagattgttgcctgtcctgtgtcaagcgcatggtctgactcccgaacaggtggtggcgattgcttcgaacaatggaggaaaacaagcattggaaaccgtccagaggctgttaccagtcctttgccaggcacatggactgaccccggaacaggtcgtcgcaatcgcatcgaacattggaggcaagcaggctctcgaaacggtccagagactgctgcctgtgctctgccaagcccacggcctcactccggagcaagtcgtggcaatcgcctcaaacatcggtggaaagcaggcccttgagactgtccagcgactgctcccagtgctctgccaggcgcatggactcacacccgagcaggtggtcgccattgctagcaacaacggaggcaaacaagccctggagacagtgcaacgcttgctgcccgtgctttgtcaagcgcacggacttactccggaacaagtagtggctattgcgagtaggcacggcggtaagcaagccctggagacagtgcagcggcttttgccggtgctctgtcaggcccacgggctcacaccggaacaggtcgtagctattgcgtcccatgacggcggaaagcaagccttagaaactgtgcagcggttgctgcctgttctgtgccaggctcatgggctcactcccgagcaagtggtcgcgattgcgagcaacggaggtggcaagcaagcactagaaaccgtgcaacgactgctgccggtcttgtgccaagcgcatggcctgacacccgaacaggtggtggctatcgcgtccaacaacgggggcaaacaggccctggaaacagtccaaaggctgttgccggtcctctgtcaagctcacggactaacccccgaacaggtggtggcaatagcttcgcggcatgggggcaagcaggcactcgaaacagtgcagaggttgctcccggtactttgccaagcacatggcttgacccctgagcaagtcgtggccatcgcatccaacggcggagggaagcaagcgcttgaaactgtccaacgcctcctgcccgtgttgtgtcaagcccatggccttacgcctgaacaagtcgtcgctattgcaagtaacggaggagggaggcctgccctggagtccattgtcgcccaactgtcgcgacccgatccagccctggcagccctcactaacgaccatctcgtggcgctggcatgcctcggtggtagaccagctctggacgccgtgaagaagggacttcctcatgcccccgcactgatcaagcgcactaaccggaggatccctgaaagaacctcccacagagtggcatccggttcaggaggagggtccggaggagctagggatgatgccaagagcttgaccgcctggtcacggactcttgtcactttcaaggatgtctttgtggactttaccagagaagaatggaagctcctggacactgcgcagcagatcctgtacagaaacgtgatgttggaaaactacaaaaacctggtgtcactcgggtaccaactcaccaagcccgacgtcatcctgcgcctggaaaagggcgaagaaccgtggttggtcgagagagaaatccatcaggaaacccaccctgactcggaaaccgccttcgagatcaagtcctcggtgcctagctccggaggaaaaaggcccgccgcaaccaagaaggctgggcaggctaagaaaaagaaggggtccggctcctaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 459 - RNA, 3120 nt, synthetic construct
  misc_feature 1..3120: note = Synthetic: MR1048, TAL37-KRAB mouse
  source 1..3120: mol_type = other RNA
ggaaataagagagaaaagaagagtaagaagaaatataagagccaccatggccccaaagaagaagcggaaagtcggaggttcaggcggctcgcacatggtggacctgagaactctgggttactcccagcaacagcaagaaaaaatcaagccaaaagtgcgcagcactgtcgcgcagcaccacgaggcccttgtggggcacgggttcacccacgctcacatcgtggccctgagccagcatccggcagccctgggaaccgtcgcagtgaagtaccaagacatgatcgccgccttgccggaagccactcacgaagcaattgtgggagtggggaagagaggagccggcgcaagagccctcgaagcgcttctgactgtggccggagaattgagaggccccccactgcagctggataccggacaactgctgaagatcgccaaacggggtggagtgaccgccgtggaagcggtgcacgcgtggagaaacgcgctgactggagccccgctcaacctgacccctgaacaggtcgtggctattgcctcaaacaacggcgggaagcaagcgttggagactgtacagcgcctgctgccagtgttgtgtcaagcccacgggctgactcccgagcaagtggtggccatagccagcaatatcggaggtaaacaggctctcgagactgtccaacgcctgctcccagtgctttgtcaggctcacggcctcactcctgaacaggtcgtagccattgccagcaacggtggcggcaaacaagccttagagactgtgcaacggctcttgccggtgctctgtcaggcccacggactgaccccagaacaagtagtcgctatcgcgtcaaacatcggcggcaagcaggctttggaaaccgtccagcggcttctgccggtgctgtgccaggcgcatgggctcacccccgaacaggtggtcgcaatcgcctcgaacggcggtggtaaacaagccctcgaaactgtccagcgcctcttgcccgtcctgtgtcaggcccatggcctgactccagaacaggtggtggcaatagcgtcccatgacgggggaaagcaagcccttgagactgtgcaacggttgctccccgtgttatgtcaggcacatggtctgacccccgaacaggtggtcgcaattgcgtcgaacggcggaggaaagcaagctctggaaacggtccaacggctgctccctgtgctgtgccaggcgcacggacttaccccagaacaagtggtcgccattgcgtccaacatcggagggaagcaggcgcttgaaaccgtgcagaggttgctgccggtgttatgccaagcacatggactaaccccggagcaagtggtggccattgcctcgaatattggggggaaacaagcgttagaaactgtgcagaggctgctgcctgtgctctgccaagcccatggtcttaccccggaacaggttgtggcgattgcatccaacaacggtggaaaacaggcccttgaaacggtgcagcggctccttcctgtcctgtgccaagcccatggactcacccctgaacaggtcgtggctattgcctccaacggaggcggcaagcaggccctggagacagtccagagactgctgcccgtgctgtgtcaagctcacggtctgactccggaacaggtggttgctatcgcttcgcacgacggcggaaaacaagctcttgaaaccgtgcaaaggctgctcccggtgctttgccaagctcatggcttgaccccagagcaagtcgtggcaatcgcgtccaatatcggtgggaagcaggctcttgagacagtgcaaagactcctccccgtcctctgtcaagcgcacgggctcactcctgaacaggtcgtcgctattgcttcgaacaacgggggcaaacaggcactagagactgtccaaagacttttacctgtcctctgccaggcacacggattgactcccgaacaagtcgtcgcgattgcctcaagacacgggggcaagcaagcgctggagactgttcagcgactgcttcctgtgctttgccaggcccacggtttgacccctgagcaagttgtggccatcgcctctaacggaggtggaaagcaggccttggaaacagtgcagagactgttgcccgtcttgtgccaggctcatgggcttacgcctgaacaagtcgtcgccatcgcttcgaacggaggagggaaacaggcgctggaaactgtgcagcgactccttccggtcctttgtcaagcacacggccttacgccggaacaagtggttgccatcgcaagccacgacggaggacgccctgccctcgagtccattgtggcccagctgtcacgcccagatcctgcactggccgctcttaccaacgatcacctggtcgctctcgcgtgcctgggaggaaggcctgccctggatgccgtcaagaagggactgcctcatgcccctgctttgattaagaggactaatcggcgcatccccgaacggaccagccacagggtggcatctgggagcggaggaggatccggcggagacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 460 - RNA, 3120 nt, synthetic construct
  misc_feature 1..3120: note = Synthetic: MR1045, TAL34-KRAB mouse
  source 1..3120: mol_type = other RNA
ggaaataagagagaaaagaagagtaagaagaaatataagagccaccatggccccaaagaagaagcggaaagtcggaggttcaggcggctcgcacatggtggacctgagaactctgggttactcccagcaacagcaagaaaaaatcaagccaaaagtgcgcagcactgtcgcgcagcaccacgaggcccttgtggggcacgggttcacccacgctcacatcgtggccctgagccagcatccggcagccctgggaaccgtcgcagtgaagtaccaagacatgatcgccgccttgccggaagccactcacgaagcaattgtgggagtggggaagagaggagccggcgcaagagccctcgaagcgcttctgactgtggccggagaattgagaggccccccactgcagctggataccggacaactgctgaagatcgccaaacggggtggagtgactgccgtcgaggctgtgcacgcctggagaaacgccttgaccggagcgccactgaaccttaccccggagcaagtggtggcgatagcatcaaatattggcggaaagcaggccctggaaaccgtacaaagactgttgccggtcctgtgtcaagctcatggtttgaccccagaacaggtcgtggctattgcatcgaacggcggaggcaaacaagccctcgagacagtccagagacttctgccggttctgtgccaggcgcacggacttacccctgaacaggtggtggcgattgcgagccacgacggtggtaaacaggcgttggaaactgtgcagcggttgcttccggtgctgtgtcaggctcacgggctgacccccgaacaagtggtagctatcgcctcccacgacggagggaagcaggctctggaaacggtgcaacgcctccttcccgtcttgtgccaagcacatggactgactccagaacaggtcgtcgccattgcctcccatgatggggggaagcaggcactggaaactgtacaacgcctgctgcctgtcctctgccaagcgcacggtttgacgccggaacaggttgtcgccatcgcctcgaacggagggggaaaacaggcccttgaaacagtgcaacggctgctgccagtgctgtgtcaggcgcatggcttgaccccggagcaagtggtggccatagcctcgaatggaggaggaaagcaggccctggaaaccgttcagcgcctcctccctgtgctgtgtcaggcccatgggctcactcccgaacaggtagtcgccatagcgtcgaacatcggaggaaaacaagcgctcgaaactgtccaaagactgcttccagtcctttgccaggctcatggactaactccagagcaagtagtggccatcgcgagccatgacggcggcaagcaagcactcgaaaccgtgcagaggctgcttcccgtcctgtgccaagctcacggcctgacgcctgaacaggtggtggctatcgccagcaacggtggtggaaagcaggcgctcgagactgtgcagaggttgctcccagtgctctgtcaggcacatggcctcaccccggagcaagtggtggcaatcgcctcaaacatcggtggcaaacaggcacttgaaaccgtgcagcgccttttgcctgtgctctgccaggcccacggactgacccctgagcaagtcgtggctattgcctctaacggcggtggcaagcaggccttggaaaccgtccagaggctcttgccggtgctctgccaggcacacggactcactccggaacaggtggtcgccatcgcatcgaatatcggcggaaaacaggccctcgaaacggtccagcggttactccccgtgctttgtcaggctcacggattgactcccgagcaagtcgtggccattgcttccaacattggggggaaacaggcgctggaaacggtgcagcgactccttcctgtgttgtgtcaagcccacggcctcactccagagcaagtcgtggcaattgcctccaacatcgggggaaagcaagccttggagacagtacagcggctgcttcctgtgctttgccaagcccatggactcacccccgaacaagtggttgccattgccagccacgatggagggaaacaagccctggagacagtgcaaaggctcctgcccgtgctgtgccaggcacacggcttgactcctgaacaagtcgtcgctatcgcttccaatggtggtgggaagcaagctctggagactgtccagcggctcctgccagtgttgtgtcaagcacacggtctgaccccggaacaagtcgttgcaatcgcgtcgcatgatggaggacgccctgccctcgagtccattgtggcccagctgtcacgcccagatcctgcactggccgctcttaccaacgatcacctggtcgctctcgcgtgcctgggaggaaggcctgccctggatgccgtcaagaagggactgcctcatgcccctgctttgattaagaggactaatcggcgcatccccgaacggaccagccacagggtggcatctgggagcggaggaggatccggcggagacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgagcggcggcaagcgcccagcggctacaaagaaggccggacaggccaagaagaagaagggttcgtacccctacgacgtgcctgactatgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 461 - protein, 362 aa, synthetic construct
  REGION 1..362: note = Synthetic: OEC695, ZF07-KRAB
MAPKKKRKVGIHGVPAAGSSGSLEPGEKPYKCPECGKSFSDKKDLTRHQRTHTGEKPYKCPECGKSFSQSSSLVRHQRTHTGEKPYKCPECGKSFSTTGNLTVHQRTHTGEKPYKCPECGKSFSTSGELVRHQRTHTGEKPYKCPECGKSFSTTGNLTVHQRTHTGEKPYKCPECGKSFSTSHSLTEHQRTHTGEKPYKCPECGKSFSTSGNLVRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 462 - protein, 956 aa, synthetic construct
  REGION 1..956: note = Synthetic: OEC693, TAL27-KRAB
MAPKKKRKVGIHGVPAAGSSGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 463 - protein, 362 aa, synthetic construct
  REGION 1..362: note = Synthetic: OEC151, ZF24-KRAB
MAPKKKRKVGIHGVPAAGSSGSLEPGEKPYKCPECGKSFSSKKHLAEHQRTHTGEKPYKCPECGKSFSQNSTLTEHQRTHTGEKPYKCPECGKSFSQSGDLRRHQRTHTGEKPYKCPECGKSFSTHLDLIRHQRTHTGEKPYKCPECGKSFSDPGNLVRHQRTHTGEKPYKCPECGKSFSTSGNLTEHQRTHTGEKPYKCPECGKSFSERSHLREHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 464 - protein, 362 aa, synthetic construct
  REGION 1..362: note = Synthetic: OEC144, ZF16-KRAB
MAPKKKRKVGIHGVPAAGSSGSLEPGEKPYKCPECGKSFSQRAHLERHQRTHTGEKPYKCPECGKSFSRSDHLTNHQRTHTGEKPYKCPECGKSFSRNDALTEHQRTHTGEKPYKCPECGKSFSRADNLTEHQRTHTGEKPYKCPECGKSFSSKKALTEHQRTHTGEKPYKCPECGKSFSRNDALTEHQRTHTGEKPYKCPECGKSFSHKNALQNHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 465 - protein, 656 aa, synthetic construct
  REGION 1..656: note = Synthetic: OEC134, ZF07-MQ1
MAPKKKRKVGIHGVPAAGSSGSLEPGEKPYKCPECGKSFSDKKDLTRHQRTHTGEKPYKCPECGKSFSQSSSLVRHQRTHTGEKPYKCPECGKSFSTTGNLTVHQRTHTGEKPYKCPECGKSFSTSGELVRHQRTHTGEKPYKCPECGKSFSTTGNLTVHQRTHTGEKPYKCPECGKSFSTSHSLTEHQRTHTGEKPYKCPECGKSFSTSGNLVRHQRTHTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 466 - protein, 656 aa, synthetic construct
  REGION 1..656: note = Synthetic: OEC129, ZF05-MQ1
MAPKKKRKVGIHGVPAAGSSGSLEPGEKPYKCPECGKSFSQSGHLTEHQRTHTGEKPYKCPECGKSFSQRANLRAHQRTHTGEKPYKCPECGKSFSTKNSLTEHQRTHTGEKPYKCPECGKSFSTSGSLVRHQRTHTGEKPYKCPECGKSFSQSGNLTEHQRTHTGEKPYKCPECGKSFSQAGHLASHQRTHTGEKPYKCPECGKSFSQSGDLRRHQRTHTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 467 - protein, 362 aa, synthetic construct
  REGION 1..362: note = Synthetic: OEC126, ZF05-KRAB
MAPKKKRKVGIHGVPAAGSSGSLEPGEKPYKCPECGKSFSQSGHLTEHQRTHTGEKPYKCPECGKSFSQRANLRAHQRTHTGEKPYKCPECGKSFSTKNSLTEHQRTHTGEKPYKCPECGKSFSTSGSLVRHQRTHTGEKPYKCPECGKSFSQSGNLTEHQRTHTGEKPYKCPECGKSFSQAGHLASHQRTHTGEKPYKCPECGKSFSQSGDLRRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 468 - protein, 956 aa, synthetic construct
  REGION 1..956: note = Synthetic: OEC103, TAL14-KRAB
MAPKKKRKVGIHGVPAAGSSGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 469 - protein, 956 aa, synthetic construct
  REGION 1..956: note = Synthetic: OEC099, TAL09-KRAB
MAPKKKRKVGIHGVPAAGSSGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 470 - protein, 956 aa, synthetic construct
  REGION 1..956: note = Synthetic: OEC095, TAL08-KRAB
MAPKKKRKVGIHGVPAAGSSGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASRHGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 471 - protein, 956 aa, synthetic construct
  REGION 1..956: note = Synthetic: OEC087, TAL04-KRAB
MAPKKKRKVGIHGVPAAGSSGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASRHGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 472 - protein, 949 aa, synthetic construct
  REGION 1..949: note = Synthetic: OEC056, TAL37-KRAB
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 473 - protein, 949 aa, synthetic construct
  REGION 1..949: note = Synthetic: OEC053, TAL34-KRAB
MAPKKKRKVGGSGGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 474 - protein, 1362 aa, synthetic construct
  REGION 1..1362: note = Synthetic: OEC043, ZF05-KRAB-tPT2A-TAL14-KRAB
MAPKKKRKVGIHGVPAAGSSGSLEPGEKPYKCPECGKSFSQSGHLTEHQRTHTGEKPYKCPECGKSFSQRANLRAHQRTHTGEKPYKCPECGKSFSTKNSLTEHQRTHTGEKPYKCPECGKSFSTSGSLVRHQRTHTGEKPYKCPECGKSFSQSGNLTEHQRTHTGEKPYKCPECGKSFSQAGHLASHQRTHTGEKPYKCPECGKSFSQSGDLRRHQRTHTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSGSATNFSLLKQAGDVEENPGPTSAGKLGSGEGRGSLLTCGDVEENPGPLEGSSGSGSPKKKRKVGIHGVPAAGSSGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASRHGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSGS